(19) United States
(12) Patent Application Publication
(10) Pub. No.: US 2024/0408085 A1
(43) Pub. Date: Dec. 12, 2024

(54) COMPOUND FOR DEGRADATION OF BCL-2 FAMILY PROTEINS AND MEDICAL APPLICATION THEREOF

(71) Applicant: XIZANG HAISCO PHARMACEUTICAL CO., LTD., Tibet (CN)

(72) Inventors: Chen ZHANG, Wenjiang District Chengdu, Sichuan (CN); Yuting LIAO, Wenjiang District Chengdu, Sichuan (CN); Yonghua LU, Wenjiang District Chengdu, Sichuan (CN); Junbin ZHAO, Wenjiang District Chengdu, Sichuan (CN); Sijia ZOU, Wenjiang District Chengdu, Sichuan (CN); Yan YU, Wenjiang District Chengdu, Sichuan (CN); Pingming TANG, Wenjiang District Chengdu, Sichuan (CN); Qiu GAO, Wenjiang District Chengdu, Sichuan (CN); Xinfan CHENG, Wenjiang District Chengdu, Sichuan (CN); Fei YE, Wenjiang District Chengdu, Sichuan (CN); Yao LI, Wenjiang District Chengdu, Sichuan (CN); Jia NI, Wenjiang District Chengdu, Sichuan (CN); Pangke YAN, Wenjiang District Chengdu, Sichuan (CN)

(21) Appl. No.: 18/688,647

(22) PCT Filed: Sep. 1, 2022
(86) PCT No.: PCT/CN2022/116529

§ 371 Date: Mar. 1, 2024

(30) Foreign Application Priority Data
Sep. 1, 2021 (CN) 202111000638.1
Oct. 20, 2021 (CN) 202111220461.6
Jan. 6, 2022 (CN) 202210003537.8
Jan. 26, 2022 (CN) 202210091103.8
May 13, 2022 (CN) 202210508260.4
Aug. 11, 2022 (CN) 202210950054.9

(51) Int. Cl. A61K 31/4995; A61K 31/427; A61K 31/454; A61P 35/00; C07D 211/14; C07D 277/22; C07D 309/26; C07D 487/08

(52) U.S. Cl. CPC A61K 31/4995; A61K 31/427; A61K 31/454; A61P 35/00; C07D 211/14; C07D 277/22; C07D 309/26; C07D 487/08

(57) ABSTRACT

A compound of general formula (I) or a stereoisomer, a deuterated compound, a solvate, a prodrug, a metabolite, a pharmaceutically acceptable salt, or a co-crystal thereof, an intermediate thereof, and a use thereof in Bcl-2 family proteins-related diseases such as cancer. B-L-K (I)

### TECHNICAL FIELD

The present invention relates to a compound of general formula (I) or a stereoisomer, deuterated compound, solvate, prodrug, metabolite, pharmaceutically acceptable salt, or co-crystal thereof, and an intermediate thereof and a preparation method therefor, as well as the use thereof in Bcl-2 family proteins-related diseases such as cancer.

### BACKGROUND ART

Apoptosis is an autonomous and orderly death process controlled by genes for most cells at certain developmental stages in the organism. It plays an important role in tissue evolution, organ development and the maintenance of the body's stability. Anti-apoptotic effects are considered a key feature in malignant tumors. Therefore, specifically targeting the anti-apoptotic pathways shows potential for cancer treatment applications. The Bcl-2 protein family consists of both pro-apoptotic and anti-apoptotic proteins, which can regulate the intrinsic apoptotic pathways of cancer cells. Bcl-2 protein family members Bcl-2, Bcl-xL and Mcl-1 have been identified as anti-tumor targets. Inhibition of these proteins can promote Bax/Bak oligomerization and ultimately induce mitochondrial outer membrane permeabilization, leading to release of cytochrome C and activation of caspases, which subsequently execute cancer cell apoptosis.

PROTAC (proteolysis targeting chimera) molecules are a class of bifunctional compounds that can simultaneously bind targeting proteins and E3 ubiquitin ligases. Such compounds can be recognized by proteasomes of cells, causing the degradation of targeting proteins, and can effectively reduce the content of targeting proteins in the cells. By introducing a ligand capable of binding to various targeting proteins into the PROTAC molecules, it is possible to apply the PROTAC technology to the treatment of various diseases, and this technology has attracted extensive attention in recent years.

Therefore, it is necessary to develop a novel Bcl-xL/Bcl-2 inhibitor and a PROTAC drug of E3 ubiquitin ligase for the treatment of apoptosis-related tumor diseases.

### SUMMARY OF THE INVENTION

An objective of the present invention is to provide a compound with a novel structure, good efficacy, high bioavailability and higher safety that can inhibit and degrade Bcl-2 family proteins (e.g., Bcl-xL or Bcl-2), for use in the treatment of a disease related to Bcl-2 family proteins (e.g., Bcl-xL or Bcl-2), such as cancer.

The present invention provides a compound or a stereoisomer, deuterated compound, solvate, prodrug, metabolite, pharmaceutically acceptable salt, or co-crystal thereof, wherein the compound is selected from a compound of general formula (I),

B-L-K (I);
in certain embodiments, L is selected from a bond or —C₁₋₅₀ hydrocarbyl-, wherein the hydrocarbyl has 0 to 20 methylene units optionally further replaced by -Ak- or -Cy-;
in certain embodiments, L is selected from a bond or —C₁₋₂₀ hydrocarbyl-, wherein the hydrocarbyl has 0 to 20 methylene units optionally further replaced by -Ak- or -Cy-;
in certain embodiments, L is selected from a bond or —C₁₋₁₀ hydrocarbyl-, wherein the hydrocarbyl has 0 to 10 (such as 0, 1, 2, 3, 4, 5, 6, 7, 8, 9 or 10) methylene units optionally further replaced by -Ak- or -Cy-;
in certain embodiments, L is selected from C₁₋₂₀ alkylene, wherein the alkylene is optionally further substituted with 0 to 4 substituents selected from H, halogen, OH, ═O, CN, NH₂, C₁₋₆ alkyl, C₁₋₆ alkoxy, halogen-substituted C₁₋₆ alkyl, hydroxyl-substituted C₁₋₆ alkyl, or cyano-substituted C₁₋₆ alkyl, the alkylene has 0 to 5 (such as 0, 1, 2, 3, 4 or 5) methylene units optionally further replaced by O, S, NH, or N(CH₃), and the alkylene chain does not contain a chemical bond selected from N—O, O—O or N—S;
in certain embodiments, L is selected from C₁₋₁₂ alkylene, wherein the alkylene is optionally further substituted with 0 to 4 substituents selected from H, halogen, OH, ═O, CN, NH₂, C₁₋₆ alkyl, C₁₋₆ alkoxy, halogen-substituted C₁₋₆ alkyl, hydroxyl-substituted C₁₋₆ alkyl, or cyano-substituted C₁₋₆ alkyl, the alkylene has 0 to 5 (such as 0, 1, 2, 3, 4 or 5) methylene units optionally further replaced by O, S, NH, or N(CH₃), and the alkylene chain does not contain a chemical bond selected from N—O, O—O or N—S;
in certain embodiments, L is selected from C₁₋₁₂ alkylene, wherein the alkylene is optionally further substituted with 0 to 4 substituents selected from H, F, OH, ═O, CN, NH₂, methyl, or methoxy, the alkylene has 0 to 5 (such as 0, 1, 2, 3, 4 or 5) methylene units optionally further replaced by O, S, NH, or N(CH₃), and the alkylene chain does not contain a chemical bond selected from N—O, O—O or N—S and optionally contains 0 to 4 (such as 0, 1, 2, 3 or 4) structural units selected from —OCH₂CH₂—, —SCH₂CH₂—, —NHCH₂CH₂—, or —N(CH₃)CH₂CH₂—;
in certain embodiments, L is selected from —C(═O)C₁₋₇ alkylene;
in certain embodiments, each -Ak- is independently selected from Ak1, Ak2, Ak3, Ak4, Ak5, Ak6, Ak7, Ak8, or Ak9;
in certain embodiments, each -Ak- is independently selected from —(CH₂)_{q}—, —(CH₂)_{q}—O—, —O—(CH₂)_{q}—, —(CH₂)_{q}—NR^{L}—, —NR^{L}—(CH₂)_{q}—, —(CH₂)_{q}—NR^{L}C(═O), —NR^{L}(CH₂)_{q}C(═O)—, —(CH₂)_{q}—C(═O)NR^{L}—, —C(═O)—, —C(═O)—(CH₂)_{q}—NR^{L}, —(C≡C)_{q}, —CH═CH—, —Si(R^{L})₂—, —Si(OH)(R^{L})—, —Si(OH)₂—, —P(═O)(OR^{L})—, —P(═O)(R^{L})—, —S—, —S(═O)—, —S(═O)₂— or a bond, wherein the —CH₂— is optionally further substituted with 0 to 2 (such as 0, 1 or 2) substituents selected from H, halogen, OH, CN, NH₂, C₁₋₆ alkyl, C₁₋₆ alkoxy, halogen-substituted C₁₋₆ alkyl, hydroxyl-substituted C₁₋₆ alkyl or cyano-substituted C₁₋₆ alkyl;
in certain embodiments, each -Cy- is independently selected from Cy1, Cy2, Cy3, Cy4 or Cy5;
in certain embodiments, each -Cy- is independently selected from a bond, a 4- to 8-membered mono-heterocyclic ring, a 4- to 10-membered fused-heterocyclic ring, a 5- to 12-membered spiro-heterocyclic ring, a 7- to 10-membered bridged-heterocyclic ring, 3- to 7-membered monocycloalkyl, 4- to 10-membered fused cycloalkyl, 5- to 12-membered spiro cycloalkyl, 7- to 10-membered bridged cycloalkyl, 5- to 10-membered heteroaryl or 6- to 10-membered aryl, wherein the aryl, heteroaryl, cycloalkyl, mono-heterocyclic ring, fused-heterocyclic ring, spiro-heterocyclic ring or bridged-heterocyclic ring is optionally further substituted with 0 to 4 (such as 0, 1, 2, 3 or 4) substituents selected from H, F, Cl, Br, I, OH, COOH, CN, NH₂, ═O, C₁₋₄ alkyl, halogen-substituted C₁₋₄ alkyl, hydroxyl-substituted C₁₋₄ alkyl or C₁₋₄ alkoxy, and the heteroaryl, mono-heterocyclic ring, fused-heterocyclic ring, spiro-heterocyclic ring or bridged-heterocyclic ring contains 1 to 4 (such as 1, 2, 3 or 4) heteroatoms selected from O, S or N, and is optionally further substituted with 0, 1 or 2 ═O when the heteroatom is selected from S;
in certain embodiments, each Cy1, Cy2, Cy3, Cy4 or Cy5 is independently selected from a bond, a 4- to 7-membered mono-heterocyclic ring, a 4- to 10-membered fused-heterocyclic ring, a 5- to 12-membered spiro-heterocyclic ring, a 7- to 10-membered bridged-heterocyclic ring, 3- to 7-membered monocycloalkyl, 4- to 10-membered fused cycloalkyl, 5- to 12-membered spiro cycloalkyl, 7- to 10-membered bridged cycloalkyl, 5- to 10-membered heteroaryl or 6- to 10-membered aryl, wherein the aryl, heteroaryl, cycloalkyl, mono-heterocyclic ring, fused-heterocyclic ring, spiro-heterocyclic ring or bridged-heterocyclic ring is optionally further substituted with 0 to 4 (such as 0, 1, 2, 3 or 4) substituents selected from H, F, Cl, Br, I, OH, COOH, CN, NH₂, ═O, C₁₋₄ alkyl, halogen-substituted C₁₋₄ alkyl, hydroxyl-substituted C₁₋₄ alkyl or C₁₋₄ alkoxy, and the heteroaryl, mono-heterocyclic ring, fused-heterocyclic ring, spiro-heterocyclic ring or bridged-heterocyclic ring contains 1 to 4 (such as 1, 2, 3 or 4) heteroatoms selected from O, S or N, and is optionally further substituted with 0, 1 or 2 ═O when the heteroatom is selected from S;
in certain embodiments, L is selected from -Cy1-Ak1-Cy2-Ak2-Cy3-Ak3-Cy4-Ak4-Cy5-Ak5-, -Cy1-Cy2-Cy3-Cy4-Ak1-Ak2-Ak3-Ak4-Ak5-, -Cy1-Ak1-Cy2-Ak2-Cy3-Ak3-Cy4-Ak4-Ak5-, -Ak1-Cy1-Ak2-Cy2-Ak3-Cy3-Ak4-Cy4-Ak5-, -Cy1-Ak1-Cy2-Ak2-Cy3-Cy4-Ak3-Ak4-Ak5-, -Cy1-Ak1-Cy2-Ak2-Ak3-Cy3-Cy4-Ak4-Ak5-, -Cy1-Ak1-Ak2-Ak3-Ak4-Ak5-Cy2-Cy3-Cy4-, -Cy1-Cy2-Ak1-Ak2-Ak3-Ak4-Ak5-Cy3-Cy4-, -Cy1-Cy2-Cy3-Ak1-Ak2-Ak3-Ak4-Ak5-Cy4-, -Cy1-Cy2-Cy3-Cy4-Ak1-Ak2-Ak3-Ak4-Ak5-, -Cy1-Ak1-Cy2-Cy3-Cy4-Ak2-Ak3-Ak4-Ak5-, -Cy1-Cy2-Ak1-Cy3-Cy4-Ak2-Ak3-Ak4-Ak5-, -Cy1-Cy2-Cy3-Ak1-Cy4-Ak2-Ak3-Ak4-Ak5-, -Cy1-Ak1-Ak2-Cy2-Cy3-Cy4-Ak3-Ak4-Ak5-, -Cy1-Cy2-Ak1-Ak2-Cy3-Cy4-Ak3-Ak4-Ak5-, -Cy1-Cy2-Cy3-Ak1-Ak2-Cy4-Ak3-Ak4-Ak5-, -Cy1-Ak1-Ak2-Ak3-Cy2-Cy3-Cy4-Ak4-Ak5-, -Cy1-Cy2-Ak1-Ak2-Ak3-Cy3-Cy4-Ak4-Ak5-, -Cy1-Cy2-Cy3-Ak1-Ak2-Ak3-Cy4-Ak4-Ak5-, -Cy1-Ak1-Ak2-Ak3-Ak4-Cy2-Cy3-Cy4-Ak5-, -Cy1-Cy2-Ak1-Ak2-Ak3-Ak4-Cy3-Cy4-Ak5-, -Cy1-Cy2-Cy3-Ak1-Ak2-Ak3-Ak4-Cy4-Ak5-, -Ak1-Ak2-Ak3-Ak4-Ak5-Cy1-Cy2-Cy3-Cy4-, -Ak1-Cy1-Cy2-Cy3-Cy4-Ak2-Ak3-Ak4-Ak5-, -Ak1-Ak2-Cy1-Cy2-Cy3-Cy4-Ak3-Ak4-Ak5-, -Ak1-Ak2-Ak3-Cy1-Cy2-Cy3-Cy4-Ak4-Ak5-, -Ak1-Ak2-Ak3-Ak4-Cy1-Cy2-Cy3-Cy4-Ak5-, -Ak1-Cy1-Ak2-Ak3-Ak4-Ak5-Cy2-Cy3-Cy4-, -Ak1-Cy1-Cy2-Ak2-Ak3-Ak4-Ak5-Cy3-Cy4-, -Ak1-Cy1-Cy2-Cy3-Ak2-Ak3-Ak4-Ak5-Cy4-, -Ak1-Ak2-Cy1-Ak3-Ak4-Ak5-Cy2-Cy3-Cy4-, -Ak1-Ak2-Cy1-Cy2-Ak3-Ak4-Ak5-Cy3-Cy4-, -Ak1-Ak2-Cy1-Cy2-Cy3-Ak3-Ak4-Ak5-Cy4-, -Ak1-Ak2-Ak3-Cy1-Ak4-Ak5-Cy2-Cy3-Cy4-, -Ak1-Ak2-Ak3-Cy1-Cy2-Ak4-Ak5-Cy3-Cy4-, -Ak1-Ak2-Ak3-Cy1-Cy2-Cy3-Ak4-Ak5-Cy4-, -Ak1-Ak2-Ak3-Ak4-Cy1-Ak5-Cy2-Cy3-Cy4-, -Ak1-Ak2-Ak3-Ak4-Cy1-Cy2-Ak5-Cy3-Cy4-, -Ak1-Ak2-Ak3-Ak4-Cy1-Cy2-Cy3-Ak5-Cy4-, -Ak1-, -Ak1-Ak2-, -Ak1-Ak2-Ak3-, -Ak1-Ak2-Ak3-Ak4-, -Ak1-Ak2-Ak3-Ak4-Ak5-, -Ak1-Ak2-Ak3-Ak4-Ak5-Ak6-, -Ak1-Ak2-Ak3-Ak4-Ak5-Ak6-Ak7-, -Ak1-Ak2-Ak3-Ak4-Ak5-Ak6-Ak7-Ak8-, or -Ak1-Ak2-Ak3-Ak4-Ak5-Ak6-Ak7-Ak8-Ak9;
in certain embodiments, L is selected from a bond, -Ak1-, -Ak1-Ak2-, -Ak1-Ak2-Ak3-, -Ak1-Ak2-Ak3-Ak4-, -Ak1-Ak2-Ak3-Ak4-Ak5-, -Ak1-Ak2-Ak3-Ak4-Ak5-Ak6-, -Cy1-, -Cy1-Ak1-, -Cy1-Ak1-Ak2-, -Cy1-Ak1-Ak2-Ak3-, -Cy1-Ak1-Ak2-Ak3-Ak4-, -Cy1-Cy2-, -Cy1-Ak1-Cy2-, -Cy1-Cy2-Ak2-, -Cy1-Ak1-Cy2-Ak2-, -Cy1-Ak1-Cy2-Ak2-Ak3-, -Cy1-Ak1-Cy2-Ak2-Ak3-Ak4-, -Cy1-Cy2-Ak2-Ak3-, -Cy1-Cy2-Ak2-Ak3-Ak4-, -Cy1-Ak1-Ak2-Cy3-, -Cy1-Ak1-Ak2-Cy3-Ak3-, -Cy1-Cy2-Cy3-, -Cy1-Ak1-Cy2-Cy3-, -Cy1-Cy2-Ak2-Cy3-, -Cy1-Cy2-Cy3-Ak3-, -Cy1-Ak1-Cy2-Cy3-Ak3-, -Cy1-Cy2-Ak2-Cy3-Ak3-, -Cy1-Ak1-Cy2-Ak2-Cy3-, -Cy1-Ak1-Cy2-Ak2-Cy3-Ak3-, -Cy1-Cy2-Cy3-Ak3-Ak4-, -Cy1-Cy2-Cy3-Ak3-Cy4-, -Cy1-Cy2-Cy3-Cy4-, -Cy1-Ak1-Cy2-Cy3-Cy4-, -Cy1-Cy2-Ak2-Cy3-Cy4-, -Cy1-Cy2-Cy3-Ak3-Cy4-, -Cy1-Cy2-Cy3-Cy4-Ak4-, -Cy1-Ak1-Cy2-Ak2-Cy3-Ak3-Cy4-, -Cy1-Ak1-Cy2-Ak2-Cy3-Cy4-, -Ak1-Cy2-, -Ak1-Cy2-Cy3-, -Ak1-Ak2-Cy3-, -Ak1-Ak2-Cy3-Cy4-, -Ak1-Cy2-Ak2-Cy3-, -Ak1-Cy2-Cy3-Ak3-Cy4-, -Ak1-Cy2-Cy3-Cy4-Ak4-Cy5-, -Ak1-Cy2-Ak2-, -Cy1-Cy2-Cy3-Ak3-Ak4-Ak5-, -Cy1-Cy2-Ak2-Cy3-Ak3-Ak4-Ak5-, -Cy1-Ak1-Cy2-Ak2-Ak3-Ak4-Ak5-, -Cy1-Cy2-Cy3-Cy4-Ak4-Ak5-, -Cy1-Ak1-Ak2-Ak3-Ak4-Ak5-, -Ak1-Cy2-Ak2-Ak3-Ak4-Ak5-, -Ak1-Cy2-Ak2-Ak3-Ak4-, -Ak1-Cy2-Ak2-Ak3-, -Ak1-Ak2-Ak3-Ak4-Ak5-, -Ak1-Ak2-Ak3-Ak4-Ak5-Ak6-, or -Ak1-Ak2-Ak3-Ak4-Ak5-Ak6-Ak7-;
in certain embodiments, L is selected from a bond or a group shown in Table B-1, wherein the left side of the group is linked to B;
in certain embodiments, L is selected from a bond, —C(═O)C₁₋₈ alkylene-, or —CH₂— 4- to 10-membered nitrogen-containing heterocycle, wherein the nitrogen-containing heterocycle is optionally substituted with 1 to 4 substituents selected from F, Cl, Br, I, OH, NH₂, COOH, CN, ═O, C₁₋₄ alkyl, halogen-substituted C₁₋₄ alkyl, hydroxyl-substituted C₁₋₄ alkyl or C₁₋₄ alkoxy, and the nitrogen-containing heterocycle contains 1 to 4 (such as 1, 2, 3 or 4) heteroatoms selected from O, S or N;
in certain embodiments, L is selected from a bond, —C(═O)C₂₋₅ alkylene-, or —CH₂— 4- to 10-membered nitrogen-containing heterocycle, wherein the nitrogen-containing heterocycle is optionally substituted with 1 to 4 substituents selected from F, Cl, Br, I, OH, NH₂, COOH, CN, ═O, methyl, or CF₃, and the nitrogen-containing heterocycle contains 1 to 4 (such as 1, 2, 3 or 4) heteroatoms selected from O, S or N;
in certain embodiments, Ak1, Ak2, Ak3, Ak4, Ak5, Ak6, Ak7, Ak8 and Ak9 are each independently selected from —(CH₂)_{q}—, —(CH₂)_{q}—O—, —O—(CH₂)_{q}—, —(CH₂)_{q}—NR^{L}, —NR^{L}—(CH₂)_{q}—, —(CH₂)_{q}—NR^{L}C(═O)—, —(CH₂)_{q}—C(═O)NR^{L}—, —C(═O)—, —C(═O)—(CH₂)_{q}—NR^{L}—, —(C≡C)_{q}— or a bond, wherein the —CH₂— is optionally further substituted with 0 to 2 (such as 0, 1 or 2) substituents selected from H, halogen, OH, CN, NH₂, C₁₋₄ alkyl, C₁₋₄ alkoxy, halogen-substituted C₁₋₄ alkyl, hydroxyl-substituted C₁₋₄ alkyl or cyano-substituted C₁₋₄ alkyl;
in certain embodiments, Ak1, Ak2, Ak3, Ak4, Ak5, Ak6, Ak7, Ak8 and Ak9 are each independently selected from —(CH₂)_{q}—, —(CH₂)_{q}—O—, —O—(CH₂)_{q}—, —(CH₂)_{q}—NR^{L}—, —NR^{L}—(CH₂)_{q}—, —(CH₂)_{q}—NR^{L}C(═O)—, —(CH₂)_{q}—C(═O)NR^{L}—, —C(═O)—, —C(═O)—(CH₂)_{q}—NR^{L}—, —(C≡C)_{q}—, or a bond, wherein the —CH₂— is optionally further substituted with 0 to 2 substituents selected from H, F, Cl, Br, I, OH, CN, NH₂, CF₃, hydroxymethyl, C₁₋₄ alkyl, or C₁₋₄ alkoxy;
in certain embodiments, Ak1, Ak2, Ak3, Ak4, Ak5, Ak6, Ak7, Ak8 and Ak9 are each independently selected from —(CH₂)_{q}—, —(CH₂)_{q}—O—, —O—(CH₂)_{q}—, —(CH₂)_{q}—NR^{L}—, —NR^{L}—(CH₂)_{q}—, —(CH₂)_{q}—NR^{L}C(═O)—, —(CH₂)_{q}—C(═O)NR^{L}—, —C(═O)—, —C(═O)—(CH₂)_{q}—NR^{L}—, —(C≡C)_{q}—, or a bond, wherein the —CH₂— is optionally further substituted with 0 to 2 substituents selected from H, F, Cl, Br, I, OH, CN, NH₂, CF₃, hydroxymethyl, methyl, ethyl, methoxy or ethoxy;
in certain embodiments, Ak1, Ak2, Ak3, Ak4, Ak5, Ak6, Ak7, Ak8 and Ak9 are each independently selected from a bond, —O—, —OCH₂—, —CH₂O—, —OCH₂CH₂—, —CH₂CH₂O—, —C≡C—, —C(CH₃)₂—, —CH₂—, —CH₂CH₂—, —CH₂CH₂CH₂—, —N(CH₃)—, —NH—, —CH₂N(CH₃)—, —CH₂NH—, —NHCH₂—, —CH₂CH₂N(CH₃)—, —CH₂CH₂NH—, —NHCH₂CH₂—, —C(═O)—, —C(═O)CH₂NH—, —CH₂C(═O)NH—, —C(═O)NH— or —NHC(═O)—;
in certain embodiments, each R^{L} is independently selected from H, C₁₋₆ alkyl, 3- to 7-membered heterocyclyl, 3- to 7-membered cycloalkyl, phenyl or 5- to 6-membered heteroaryl;
in certain embodiments, each R^{L} is independently selected from H or C₁₋₆ alkyl;
in certain embodiments, each R^{L} is independently selected from H or C₁₋₄ alkyl;
in certain embodiments, each R^{L} is independently selected from H, methyl or ethyl;
in certain embodiments, each Cy1, Cy2, Cy3, Cy4 or Cy5 is independently selected from a bond or one of the following substituted or unsubstituted groups: cyclopropyl, cyclobutyl, cyclopentyl, cyclohexyl, azetidinyl, azacyclopentyl, azacyclohexenyl, piperidyl, morpholinyl, piperazinyl, phenyl, cyclopropyl-fused-cyclopropyl, cyclopropyl-fused-cyclobutyl, cyclopropyl-fused-cyclopentyl, cyclopropyl-fused-cyclohexyl, cyclobutyl-fused-cyclobutyl, cyclobutyl-fused-cyclopentyl, cyclobutyl-fused-cyclohexyl, cyclopentyl-fused-cyclopentyl, cyclopentyl-fused-cyclohexyl, cyclohexyl-fused-cyclohexyl, cyclopropyl-spiro-cyclopropyl, cyclopropyl-spiro-cyclobutyl, cyclopropyl-spiro-cyclopentyl, cyclopropyl-spiro-cyclohexyl, cyclobutyl-spiro-cyclobutyl, cyclobutyl-spiro-cyclopentyl, cyclobutyl-spiro-cyclohexyl, cyclopentyl-spiro-cyclopentyl, cyclopentyl-spiro-cyclohexyl, cyclohexyl-spiro-cyclohexyl, cyclopropyl-fused-azetidinyl, cyclopropyl-fused-azacyclopentyl, cyclopropyl-fused-azacyclohexyl, cyclopropyl-fused-piperidyl, cyclobutyl-fused-azetidinyl, cyclobutyl-fused-azacyclopentyl, cyclobutyl-fused-azacyclohexyl, cyclobutyl-fused-piperidyl, cyclopentyl-fused-azetidinyl, cyclopentyl-fused-azacyclopentyl, cyclopentyl-fused-azacyclohexyl, cyclopentyl-fused-piperidyl, cyclohexyl-fused-azetidinyl, cyclohexyl-fused-azacyclopentyl, cyclohexyl-fused-azacyclohexyl, cyclohexyl-fused-piperidyl, azetidinyl-fused-azetidinyl, azetidinyl-fused-azacyclopentyl, azetidinyl-fused-azacyclohexyl, azetidinyl-fused-piperidyl, azacyclopentyl-fused-azetidinyl, azacyclopentyl-fused-azacyclopentyl, azacyclopentyl-fused-azacyclohexyl, azacyclopentyl-fused-piperidyl, azacyclohexyl-fused-azetidinyl, azacyclohexyl-fused-azacyclopentyl, azacyclohexyl-fused-azacyclohexyl, azacyclohexyl-fused-piperidyl, cyclobutyl-spiro-azetidinyl, cyclobutyl-spiro-azacyclopentyl, cyclobutyl-spiro-azacyclohexyl, cyclopentyl-spiro-azetidinyl, cyclopentyl-spiro-azacyclopentyl, cyclopentyl-spiro-azacyclohexyl, cyclohexyl-spiro-azetidinyl, cyclohexyl-spiro-azacyclopentyl, cyclohexyl-spiro-azacyclohexyl, azetidinyl-spiro-azetidinyl, azetidinyl-spiro-azacyclopentyl, azetidinyl-spiro-azacyclohexyl, azacyclopentyl-spiro-azetidinyl, azacyclopentyl-spiro-azacyclopentyl, azacyclopentyl-spiro-azacyclohexyl, azacyclohexyl-spiro-azetidinyl, azacyclohexyl-spiro-azacyclopentyl, azacyclohexyl-spiro-azacyclohexyl, cyclobutyl-spiro-piperidyl, cyclopentyl-spiro-piperidyl, cyclohexyl-spiro-piperidyl, azetidinyl-spiro-piperidyl, azacyclopentyl-spiro-piperidyl, azacyclohexyl-spiro-piperidyl,

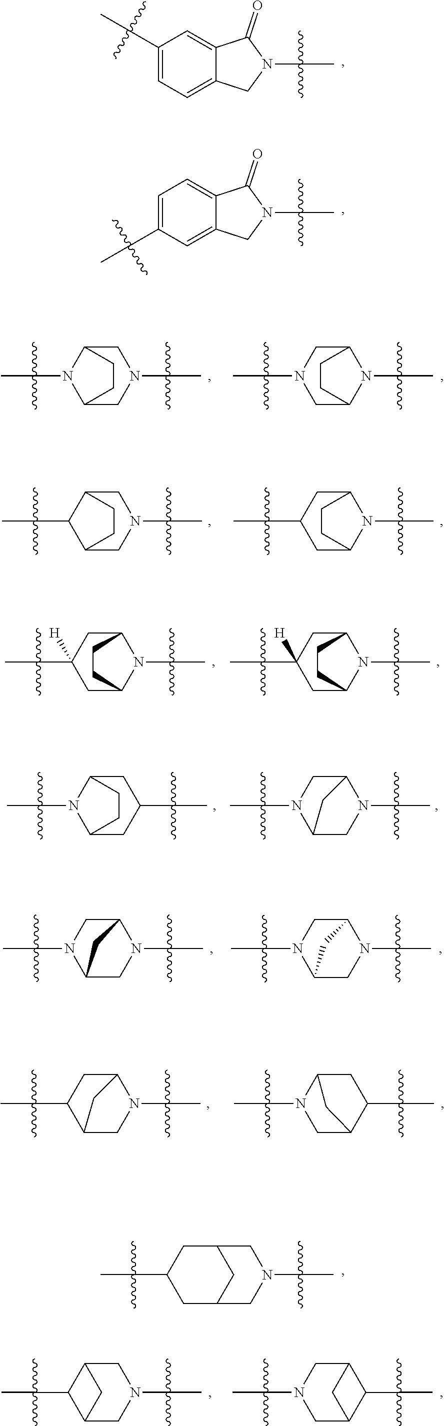

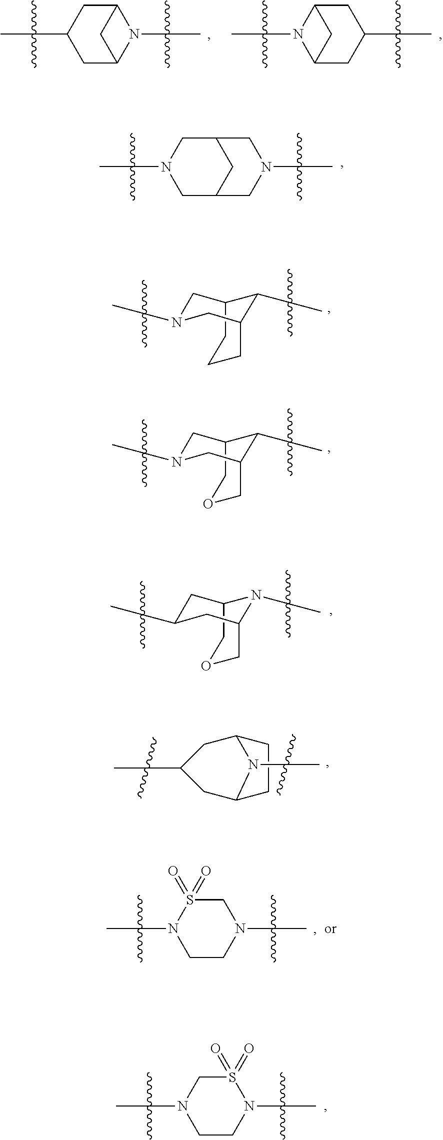

which, when substituted, is optionally further substituted with 0 to 4 (such as 0, 1, 2, 3 or 4) substituents selected from H, F, Cl, Br, I, OH, NH₂, COOH, CN, ═O, C₁₋₄ alkyl, halogen-substituted C₁₋₄ alkyl, hydroxyl-substituted C₁₋₄ alkyl, or C₁₋₄ alkoxy;
in certain embodiments, each Cy1, Cy2, Cy3, Cy4 or Cy5 is independently selected from a bond or one of the following substituted or unsubstituted groups:

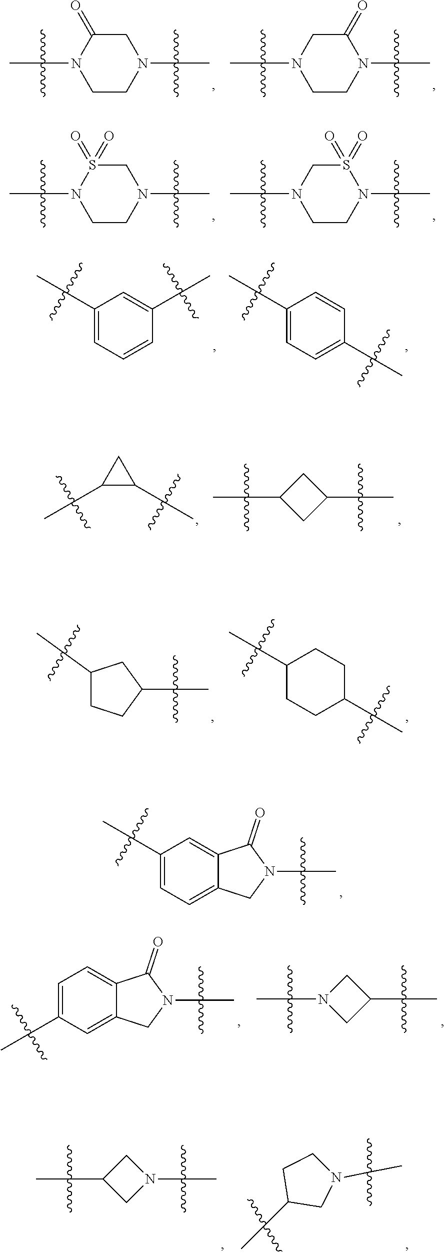

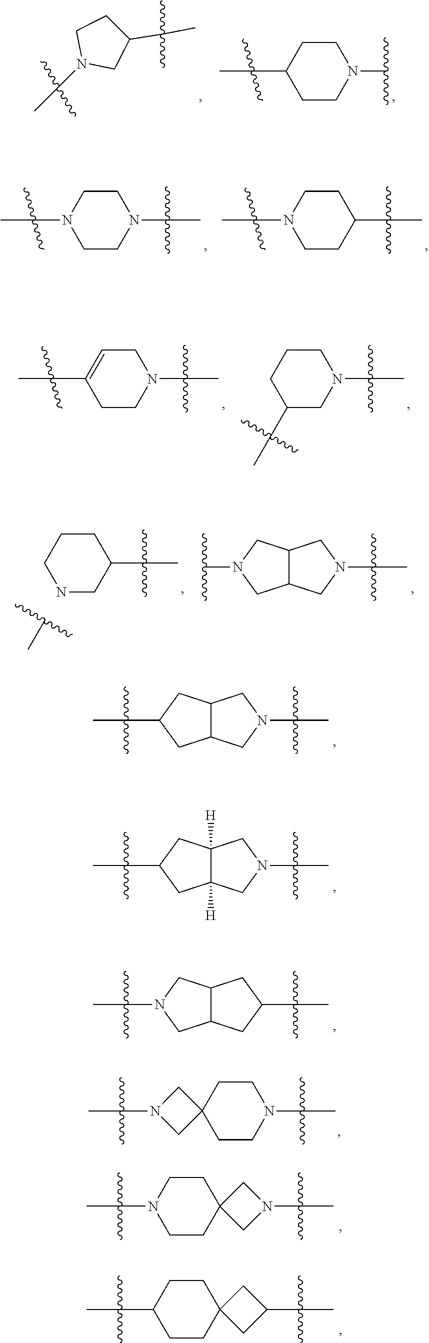

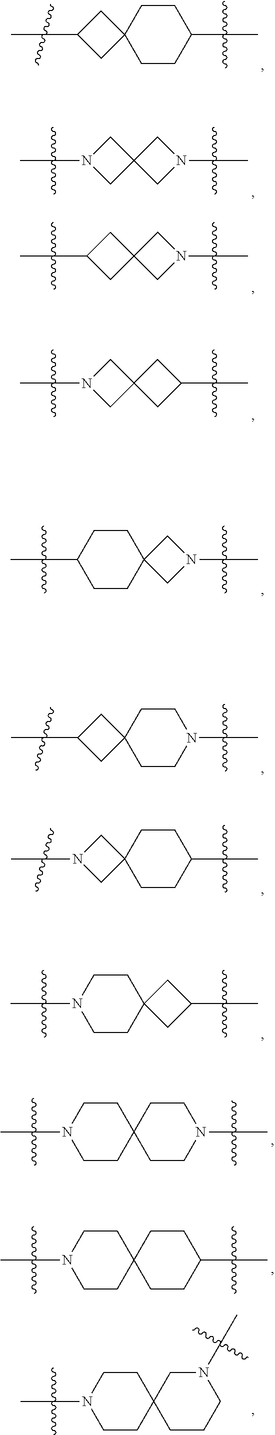

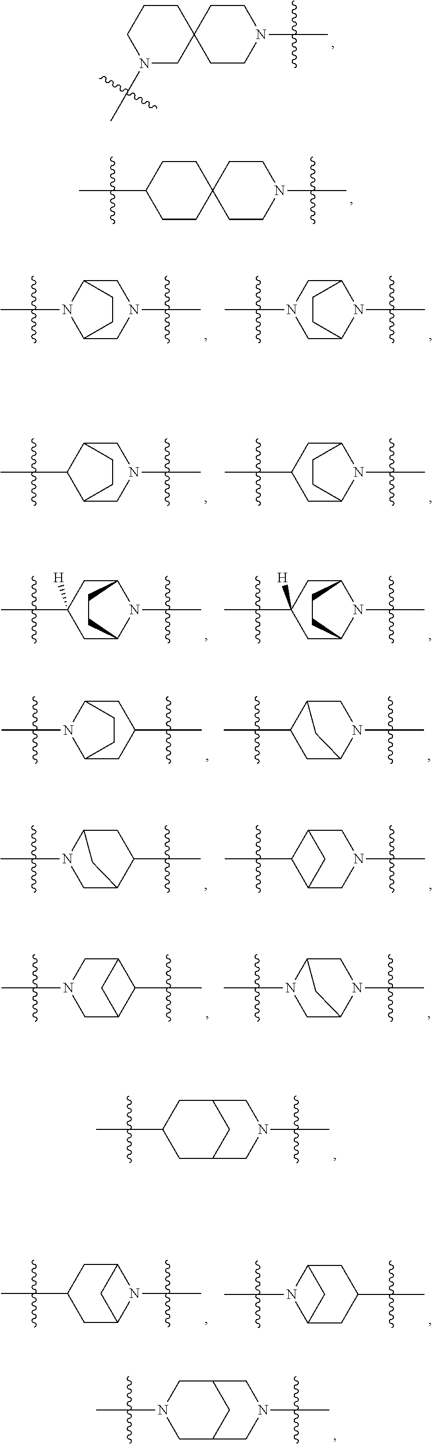

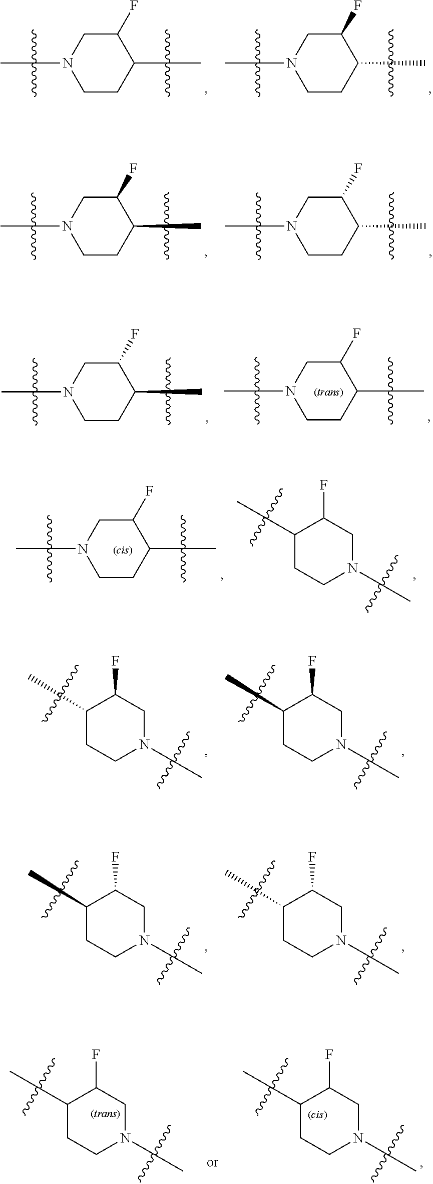

which, when substituted, is optionally further substituted with 0 to 4 (such as 0, 1, 2, 3 or 4) substituents selected from H, F, CF₃, methyl, ═O, hydroxymethyl, COOH, CN or NH₂;
in certain embodiments, K is selected from

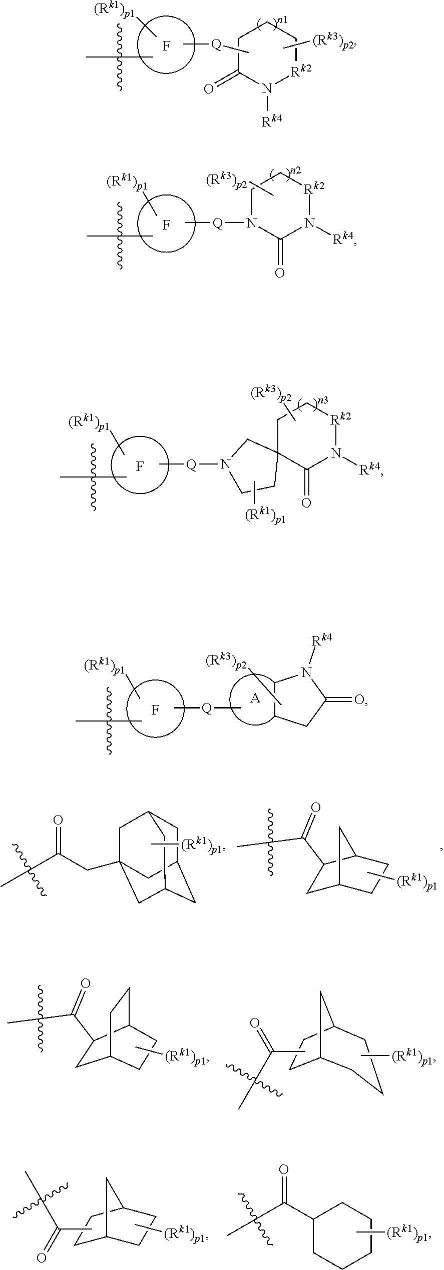

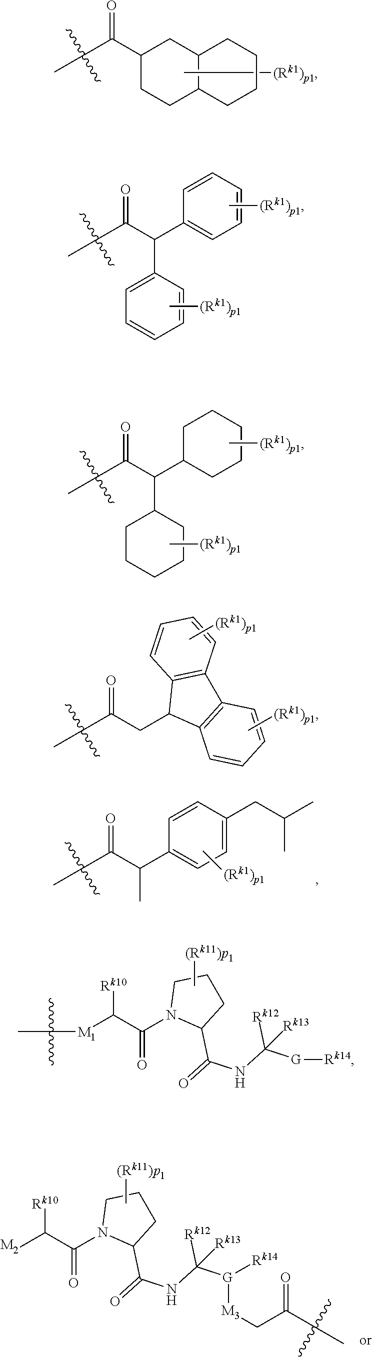

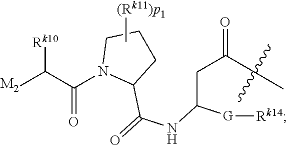

in certain embodiments, K is selected from

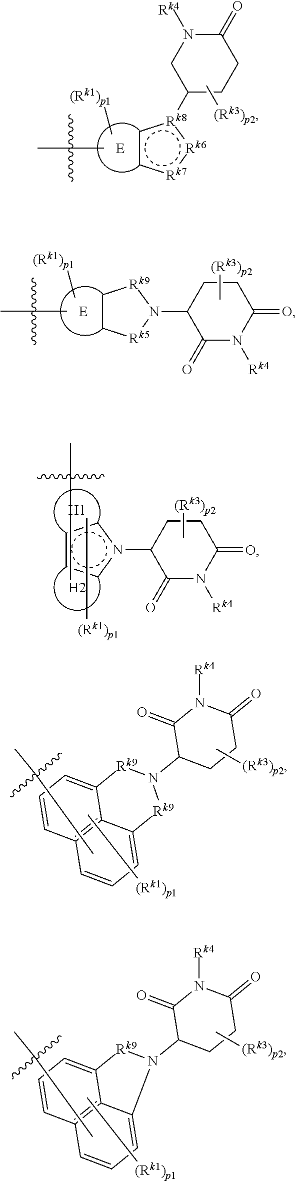

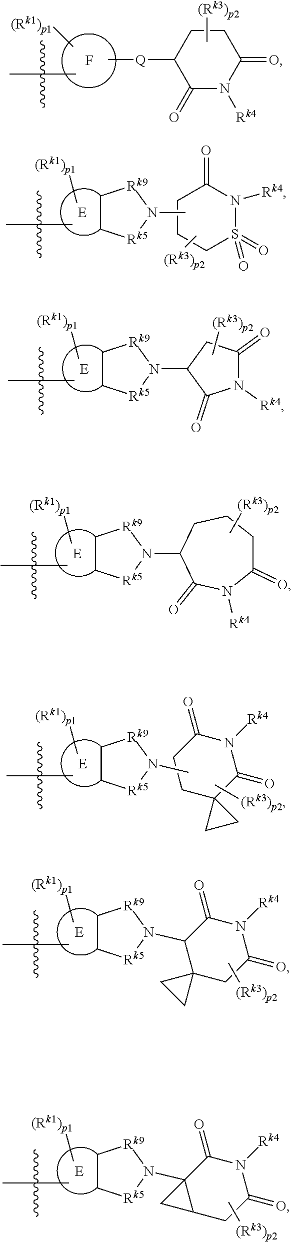

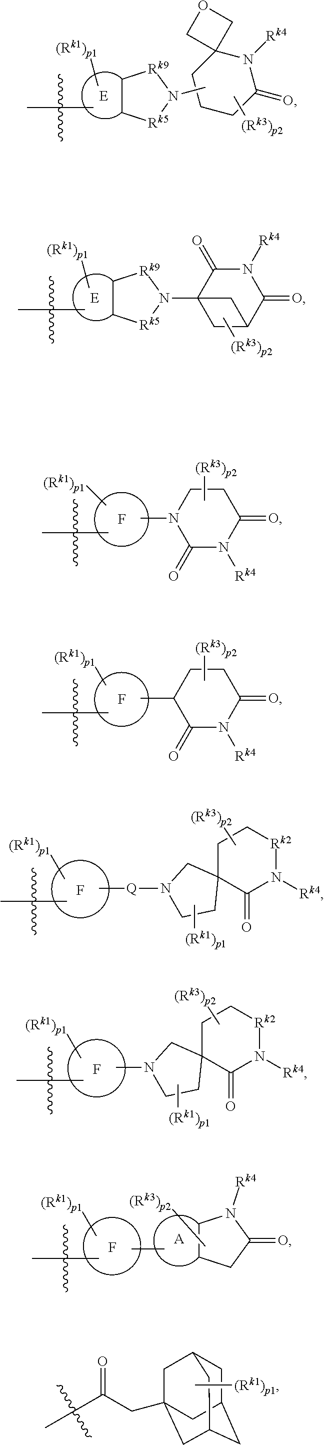

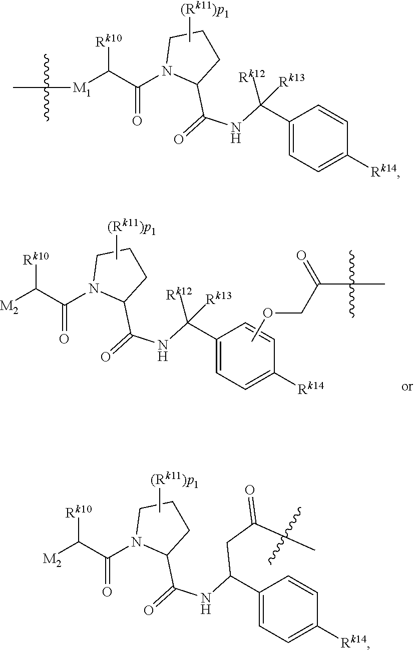

wherein [symbol] represents a ring selected from an aromatic ring or a non-aromatic ring;
in certain embodiments, K is selected from

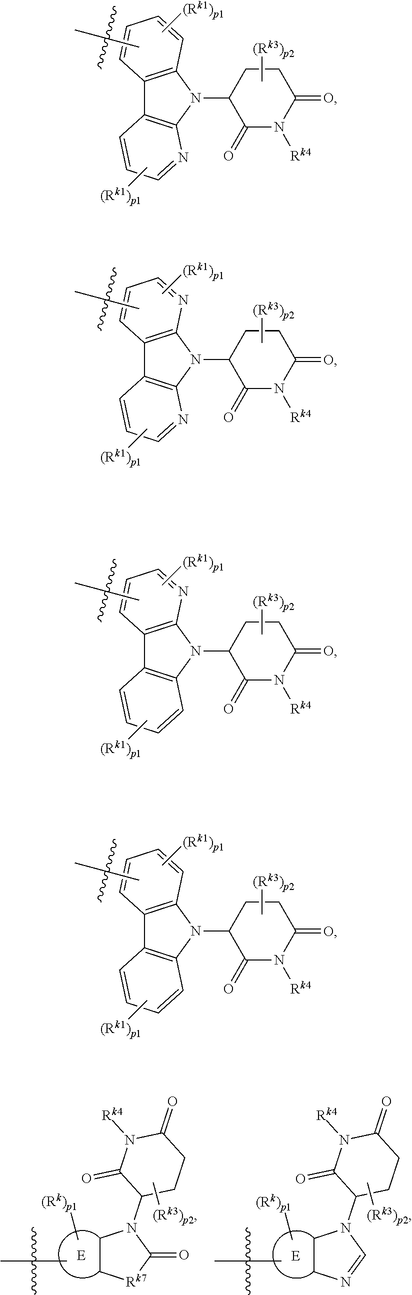

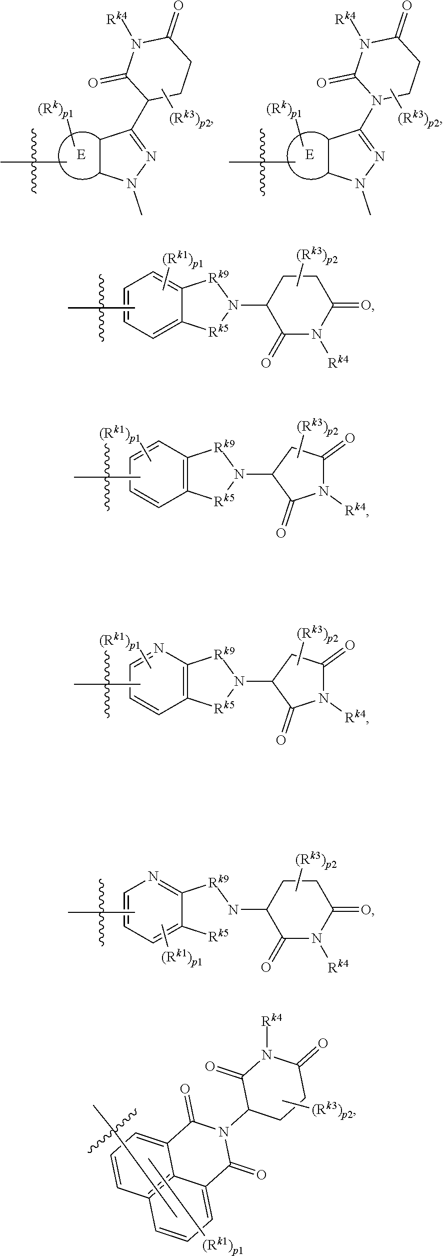

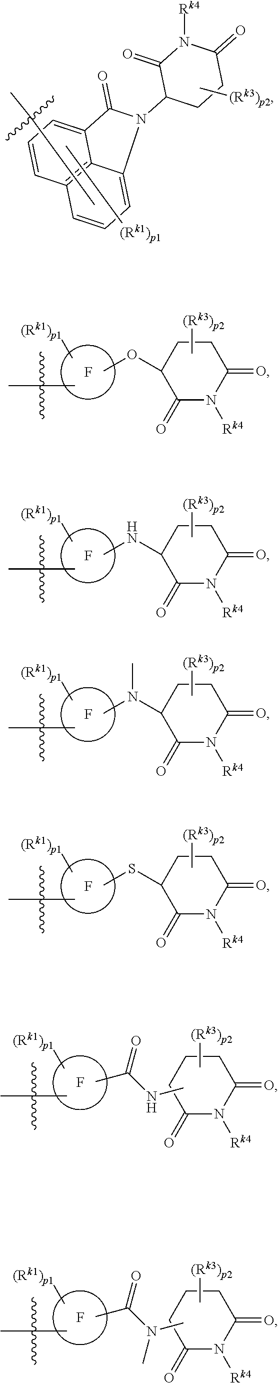

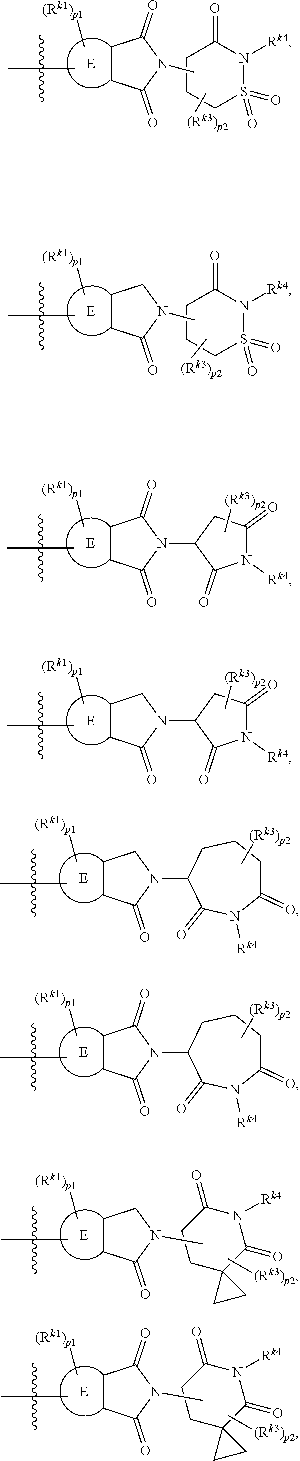

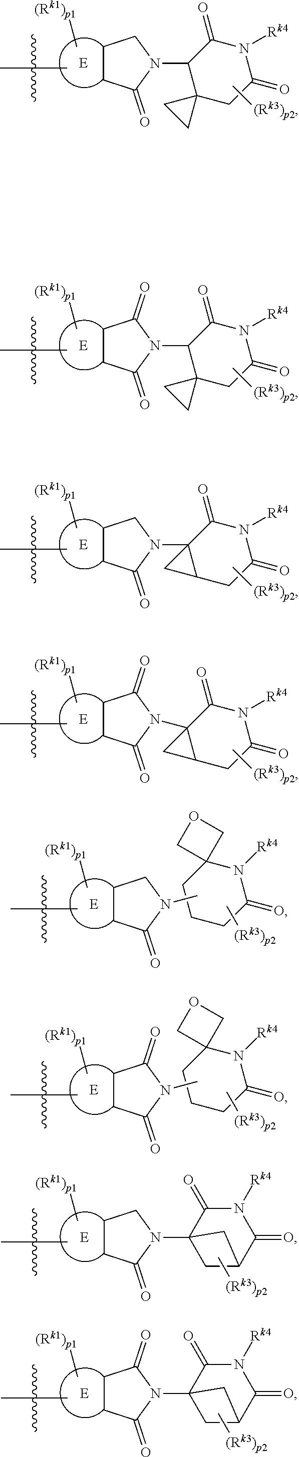

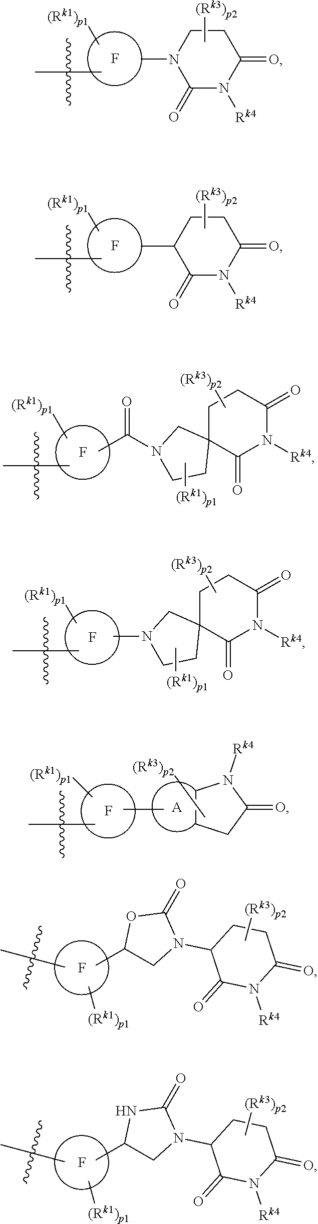

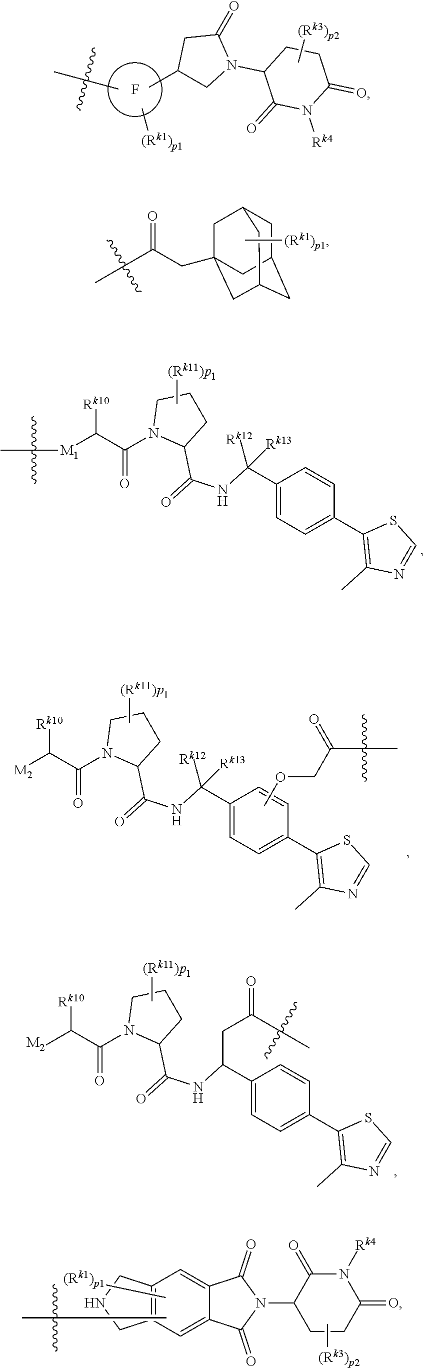

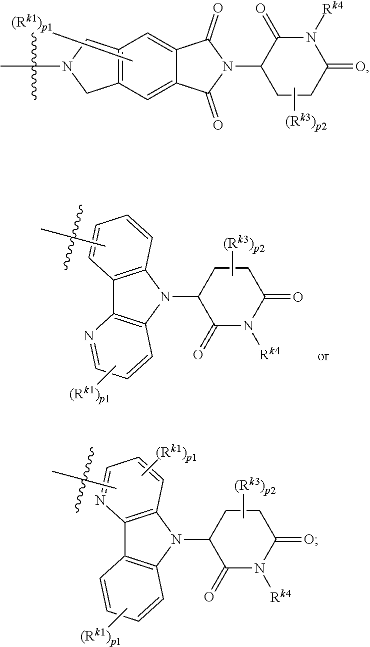

in certain embodiments, each Q is independently selected from a bond, —O—, —S—, —CH₂—, —NR^{q}—, —CO—, —NR^{q}CO—, —CONR^{q}— or 3- to 12-membered heterocyclyl, wherein the heterocyclyl is optionally further substituted with 0 to 4 (such as 0, 1, 2, 3 or 4) substituents selected from H, F, Cl, Br, I, OH, ═O, NH₂, CN, COOH, CONH₂, C₁₋₄ alkyl or C₁₋₄ alkoxy, and the heterocyclyl contains 1 to 4 (such as 1, 2, 3 or 4) heteroatoms selected from O, S or N;
in certain embodiments, each Q is independently selected from —O—, —S—, —CH₂—, —NR^{q}—, —CO—, —NR^{q}CO—, —CONR^{q}— or 4- to 7-membered heterocyclyl, wherein the heterocyclyl is optionally further substituted with 0 to 4 (such as 0, 1, 2, 3 or 4) substituents selected from H, F, Cl, Br, I, OH, ═O, NH₂, CN, COOH, CONH₂, C₁₋₄ alkyl or C₁₋₄ alkoxy, and the heterocyclyl contains 1 to 4 (such as 1, 2, 3 or 4) heteroatoms selected from O, S or N;
in certain embodiments, R^{q} is selected from H or C₁₋₆ alkyl;
in certain embodiments, R^{q} is selected from H or C₁₋₄ alkyl;
in certain embodiments, R^{q} is selected from H, methyl or ethyl;
in certain embodiments, each E is independently selected from C₃₋₁₀ carbocyclyl, C₆₋₁₀ aryl, 3- to 12-membered heterocyclyl or 5- to 12-membered heteroaryl, wherein the heterocycle or heteroaryl contains 1 to 4 (such as 1, 2, 3 or 4) heteroatoms selected from O, S or N;
in certain embodiments, each E is independently selected from C₃₋₈ carbocycle, a benzene ring, 4- to 7-membered heterocycle, 8- to 12-membered heterocycle, 7- to 12-membered heteroaryl or 5- to 6-membered heteroaryl, wherein the heterocycle or heteroaryl contains 1 to 4 (such as 1, 2, 3 or 4) heteroatoms selected from O, S or N;
in certain embodiments, each E is independently selected from phenyl, pyridyl, pyridazinyl, pyrazinyl, pyrimidyl, pyrrolyl, pyrazolyl, imidazolyl, thiazolyl, furyl, thienyl, oxazolyl, indolinyl, isoindolinyl, 1,2,3,4-tetrahydroquinolyl or 1,2,3,4-tetrahydroisoquinolinyl;
in certain embodiments, each E is independently selected from phenyl, pyridyl, pyridazinyl, pyrazinyl, pyrimidyl, pyrrolyl, pyrazolyl, imidazolyl, thiazolyl, furyl, thienyl or oxazolyl;
in certain embodiments, each E is independently selected from phenyl, pyridyl, pyridazinyl, pyrazinyl or pyrimidyl;
in certain embodiments, each E is independently selected from a benzene ring or a pyridine ring;
in certain embodiments, A is selected from C₃₋₁₀ carbocyclyl, C₆₋₁₀ aryl, 3- to 10-membered heterocyclyl or 5- to 10-membered heteroaryl, wherein the heterocycle or heteroaryl contains 1 to 4 (such as 1, 2, 3 or 4) heteroatoms selected from O, S or N;
in certain embodiments, each A, H1 or H2 is independently selected from C₃₋₈ carbocycle, a benzene ring, 4- to 7-membered heterocycle or a 5- to 6-membered heteroaryl, wherein the heterocycle or heteroaryl contains 1 to 4 (such as 1, 2, 3 or 4) heteroatoms selected from O, S or N;
in certain embodiments, each A, H1 or H2 is independently selected from phenyl, pyridyl, pyridazinyl, pyrazinyl, pyrimidyl, pyrrolyl, pyrazolyl, imidazolyl, thiazolyl, furyl, thienyl or oxazolyl;
in certain embodiments, each A, H1 or H2 is independently selected from phenyl or pyridyl;
in certain embodiments, each F (ring F) is independently selected from C₃₋₂₀ carbocyclyl, C₆₋₂₀ aryl, 3- to 20-membered heterocyclyl or 5- to 20-membered heteroaryl, wherein the heterocyclyl or heteroaryl contains 1 to 4 (such as 1, 2, 3 or 4) heteroatoms selected from O, S or N;
in certain embodiments, each F is independently selected from 3- to 7-membered monocycloalkyl, 4- to 10-membered fused cycloalkyl, 5- to 12-membered spiro cycloalkyl, 5- to 10-membered bridged cycloalkyl, 4- to 7-membered mono-heterocyclic ring, 4- to 10-membered fused-heterocyclic ring, 5- to 12-membered spiro-heterocyclic ring, 5- to 10-membered bridged-heterocyclic ring, C₆₋₁₄ aryl or 5- to 10-membered heteroaryl, wherein the mono-heterocyclic ring, fused-heterocyclic ring, spiro-heterocyclic ring, bridged-heterocyclic ring or heteroaryl contains 1 to 4 (such as 1, 2, 3 or 4) heteroatoms selected from O, S or N;
in certain embodiments, each F is independently selected from cyclopropyl, cyclobutyl, cyclopentyl, cyclohexyl, bicyclo[1.1.1]pentanyl, 6,7-dihydro-5H-cyclopenta[c]pyridyl, 2,3-dihydro-1H-indenyl, phenyl, naphthyl, anthryl, phenanthryl, azetidinyl, azacyclopentyl, piperidyl, morpholinyl, pyridyl, pyrimidyl, pyridazinyl, pyrazinyl, triazinyl, pyrrolyl, pyrazolyl, imidazolyl, triazolyl, oxazolyl, furyl, thienyl, thiazolyl, benzoimidazolyl, benzopyrazolyl, benzothiazolyl, benzothienyl, benzofuryl, benzopyrrolyl, benzopyridyl, benzopyrazinyl, benzopyrimidyl, benzopyridazinyl, pyrrolopyrrolyl, pyrrolopyridyl, pyrrolopyrimidyl, pyrrolopyridazinyl, pyrrolopyrazinyl, imidazopyrimidyl, imidazopyridyl, imidazopyrazinyl, imidazopyridazinyl, pyrazolopyridyl, pyrazolopyrimidyl, pyrazolopyridazinyl, pyrazolopyrazinyl, pyrimidopyridyl, pyrimidopyrazinyl, pyrimidopyridazinyl, pyrimidopyrimidyl, pyridopyridyl, pyridopyrazinyl, pyridopyridazinyl, pyridazinopyridazinyl, pyridazinopyrazinyl or pyrazinopyrazinyl;
in certain embodiments, each R^{k2} is independently selected from a bond, —CO—, —SO₂—, —SO— or —C(R^{k3})₂—;
in certain embodiments, each R^{k2} is independently selected from —CO—, —SO₂— or —C(R^{k3})₂—;
in certain embodiments, each R^{k1} is independently selected from H, F, Cl, Br, I, OH, ═O, NH₂, CN, COOH, CONH₂, C₁₋₆ alkyl or C₁₋₆ alkoxy, wherein the alkyl or alkoxy is optionally further substituted with 0 to 4 (such as 0, 1, 2, 3 or 4) substituents selected from H, F, Cl, Br, I, OH, ═O, NH₂, CN, COOH, CONH₂, C₁₋₄ alkyl or C₁₋₄ alkoxy;
in certain embodiments, each R^{k3} is independently selected from H, F, Cl, Br, I, OH, ═O, NH₂, CN, COOH, CONH₂, C₁₋₆ alkyl, C₁₋₆ alkoxy, C₃₋₈ cycloalkyl or 3- to 8-membered heterocyclyl, wherein the alkyl, alkoxy, cycloalkyl or heterocyclyl is optionally further substituted with 0 to 4 (such as 0, 1, 2, 3 or 4) substituents selected from H, F, Cl, Br, I, OH, ═O, NH₂, CN, COOH, CONH₂, C₁₋₄ alkyl or C₁₋₄ alkoxy, and the heterocyclyl contains 1 to 4 (such as 1, 2, 3 or 4) heteroatoms selected from O, S or N;
in certain embodiments, R^{k1} and R^{k3} are each independently selected from H, F, Cl, Br, I, OH, ═O, NH₂, CF₃, CN, COOH, CONH₂, C₁₋₄ alkyl or C₁₋₄ alkoxy, wherein the alkyl or alkoxy is optionally further substituted with 0 to 4 (such as 0, 1, 2, 3 or 4) substituents selected from H, F, Cl, Br, I, OH or NH₂;
in certain embodiments, R^{k1} and R^{k3} are each independently selected from H, F, Cl, Br, I, OH, ═O, NH₂, CF₃, CN, COOH, CONH₂, methyl, ethyl, isopropyl, methoxy, ethoxy or isopropoxy, wherein the methyl, ethyl, isopropyl, methoxy, ethoxy or isopropoxy is optionally further substituted with 0 to 4 (such as 0, 1, 2, 3 or 4) substituents selected from H, F, Cl, Br, I, OH or NH₂;
in certain embodiments, two R^{k3} together with the carbon atoms or ring backbones to which they are directly attached form 3- to 8-membered carbocycle or 3- to 8-membered heterocycle, wherein the carbocycle or heterocycle is optionally further substituted with 0 to 4 (such as 0, 1, 2, 3 or 4) substituents selected from H, F, Cl, Br, I, OH, ═O, NH₂, CN, COOH, CONH₂, C₁₋₄ alkyl or C₁₋₄ alkoxy, and the heterocycle contains 1 to 4 (such as 1, 2, 3 or 4) heteroatoms selected from O, S or N;
in certain embodiments, two R^{k3} together with the carbon atoms or ring backbones to which they are directly attached form 3- to 6-membered carbocycle or 3- to 7-membered heterocycle, wherein the carbocycle or heterocycle is optionally further substituted with 0 to 4 (such as 0, 1, 2, 3 or 4) substituents selected from H, F, Cl, Br, I, OH, ═O, NH₂, CN, COOH, CONH₂, C₁₋₄ alkyl or C₁₋₄ alkoxy, and the heterocycle contains 1 to 4 (such as 1, 2, 3 or 4) heteroatoms selected from O, S or N;
in certain embodiments, each R^{k4} is independently selected from H, OH, NH₂, CN, CONH₂, C₁₋₆ alkyl, C₃₋₈ cycloalkyl or 3- to 8-membered heterocyclyl, wherein the alkyl, cycloalkyl or heterocyclyl is optionally further substituted with 0 to 4 (such as 0, 1, 2, 3 or 4) substituents selected from H, F, Cl, Br, I, OH, ═O, NH₂, CN, COOH, CONH₂, C₁₋₄ alkyl or C₁₋₄ alkoxy, and the heterocyclyl contains 1 to 4 (such as 1, 2, 3 or 4) heteroatoms selected from O, S or N;
in certain embodiments, each R^{k4} is independently selected from H, OH, NH₂, CF₃, CN or C₁₋₄ alkyl;
in certain embodiments, each R^{k5} is independently selected from CO, CH₂, SO₂ or

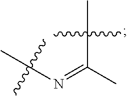 [SMILES: */N=C(\*)C]

in certain embodiments, each R^{k6} is independently selected from CO, CH, SO, SO₂, CH₂ or N;
in certain embodiments, each R^{k7} is independently selected from CO, CH, N, CH₂, O, S, N(CH₃) or NH;
in certain embodiments, each R^{k7} is independently selected from CH₂, O, N(CH₃) or NH;
in certain embodiments, each R^{k8} is independently selected from C, N or CH;
in certain embodiments, each R^{k9} is independently selected from CO, SO₂ or CH₂;
in certain embodiments, M₁ is selected from a bond, —C(═O)NH—, —NHC(═O)—, —CH₂—C(═O)NH—, —C(═O)CH₂NH—, or 5- to 6-membered heteroaryl, wherein the heteroaryl is optionally further substituted with 0 to 4 (such as 0, 1, 2, 3 or 4) substituents selected from H, F, Cl, Br, I, OH, ═O, CF₃, NH₂, CN, C₁₋₄ alkyl, halogen-substituted C₁₋₄ alkyl, hydroxyl-substituted C₁₋₄ alkyl, or C₁₋₄ alkoxy, and the heteroaryl contains 1 to 4 (such as 1, 2, 3 or 4) heteroatoms selected from O, S or N;
in certain embodiments, M1 is selected from a bond, —C(═O)NH—, —CH₂—C(═O)NH—, —C(═O)CH₂NH—, pyrrolyl, pyrazolyl, imidazolyl, triazolyl, oxazolyl, isoxazolyl, furyl, thienyl, or thiazolyl;
in certain embodiments, M₁ is selected from a bond, —CH₂—C(═O)NH— or —C(═O)CH₂NH—;
in certain embodiments, M₂ is selected from —NHC(═O)—C₁₋₆ alkyl, —NHC(═O)—C₃₋₆ cycloalkyl or 4- to 10-membered heterocyclyl, wherein the alkyl, cycloalkyl or heterocyclyl is optionally further substituted with 0 to 4 (such as 0, 1, 2, 3 or 4) substituents selected from H, F, Cl, Br, I, ═O, OH, NH₂, C₁₋₄ alkyl or C₁₋₄ alkoxy, and the heterocyclyl contains 1 to 4 heteroatoms selected from O, S or N;
in certain embodiments, M₂ is selected from —NHC(═O)—C₁₋₄ alkyl, —NHC(═O)—C₃₋₆ cycloalkyl or 4- to 10-membered heterocyclyl, wherein the alkyl, cycloalkyl or heterocyclyl is optionally further substituted with 0 to 4 (such as 0, 1, 2, 3 or 4) substituents selected from H, F, Cl, Br, I, ═O, OH, NH₂, C₁₋₄ alkyl or C₁₋₄ alkoxy, and the heterocyclyl contains 1 to 4 heteroatoms selected from O, S or N;
in certain embodiments, M₂ is selected from —NHC(═O)—CH₃, —NHC(═O)-cyclopropyl, —NHC(═O)-cyclobutyl, azetidinyl, azacyclopentyl, benzo-azacyclopentyl or benzo-azacyclohexyl, wherein the cyclopropyl, cyclobutyl, azetidinyl, azacyclopentyl, benzo-azacyclopentyl or benzo-azacyclohexyl is optionally further substituted with 0 to 4 (such as 0, 1, 2, 3 or 4) substituents selected from H, F, Cl, Br, I, ═O, OH, NH₂, C₁₋₄ alkyl or C₁₋₄ alkoxy;
in certain embodiments, M₃ is selected from —NH— or —O—;
in certain embodiments, R^{k10} is selected from C₁₋₆ alkyl, wherein the alkyl is optionally further substituted with 0 to 4 (such as 0, 1, 2, 3 or 4) substituents selected from H, F, Cl, Br, I, ═O, OH, C₁₋₆ alkyl or C₃₋₆ cycloalkyl;
in certain embodiments, R^{k10} is selected from C₁₋₄ alkyl, wherein the alkyl is optionally further substituted with 0 to 4 (such as 0, 1, 2, 3 or 4) substituents selected from H, F, Cl, Br, I, ═O, OH, C₁₋₄ alkyl or C₃₋₆ cycloalkyl;
in certain embodiments, R^{k10} is selected from methyl, ethyl, isopropyl, propyl, or tert-butyl, wherein the methyl, ethyl, isopropyl, propyl, or tert-butyl is optionally further substituted with 0 to 4 (such as 0, 1, 2, 3 or 4) substituents selected from H, F, Cl, Br, I, ═O, OH, C₁₋₄ alkyl or C₃₋₆ cycloalkyl;
in certain embodiments, G is selected from 6- to 10-membered aryl or 5- to 10-membered heteroaryl, wherein the aryl or heteroaryl is optionally further substituted with 0 to 4 (for example, 0, 1, 2, 3 or 4) substituents selected from H, F, Cl, Br, I, OH, ═O, CF₃, CN, C₁₋₄ alkyl, halogen-substituted C₁₋₄ alkyl, hydroxyl-substituted C₁₋₄ alkyl, C₁₋₄ alkoxy or C₃₋₆ cycloalkyl, and the heteroaryl contains 1 to 4 (for example, 1, 2, 3 or 4) heteroatoms selected from N, O or S;
in certain embodiments, each R^{k11} is independently selected from H, F, Cl, Br, I, ═O, OH, SH, C₁₋₆ alkyl, C₁₋₆ alkoxy or C₁₋₆ alkylthio or —O—C(═O)—C₁₋₆ alkyl, wherein the alkyl, alkoxy or alkylthio is optionally further substituted with 0 to 4 (such as 0, 1, 2, 3 or 4) substituents selected from H, F, Cl, Br, I, OH, C₁₋₄ alkyl or C₁₋₄ alkoxy;
in certain embodiments, each R^{k11} is independently selected from H, F, Cl, Br, I, ═O, OH, SH, C₁₋₄ alkyl, C₁₋₄ alkoxy, or C₁₋₄ alkylthio or —O—C(═O)—C₁₋₄ alkyl, wherein the alkyl, alkoxy or alkylthio is optionally further substituted with 0 to 4 (such as 0, 1, 2, 3 or 4) substituents selected from H, F, Cl, Br, I, OH, C₁₋₄ alkyl or C₁₋₄ alkoxy;
in certain embodiments, each R^{k11} is independently selected from H, F, Cl, Br, I, ═O, OH, SH, methyl, ethyl, isopropyl, propyl, methoxy, ethoxy, propoxy, isopropyloxy, methylthio, ethylthio, propylthio or —O—C(═O)—CH₃, wherein the methyl, ethyl, isopropyl, propyl, methoxy, ethoxy, propoxy, isopropyloxy, methylthio, ethylthio, or propylthio is optionally further substituted with 0 to 4 (such as 0, 1, 2, 3 or 4) substituents selected from H, F, Cl, Br, I, OH, C₁₋₄ alkyl or C₁₋₄ alkoxy;
in certain embodiments, R^{k12} and R^{k13} are each independently selected from H, C₁₋₆ alkyl or C₃₋₆ cycloalkyl, wherein the alkyl or cycloalkyl is optionally further substituted with 0 to 4 (such as 0, 1, 2, 3 or 4) substituents selected from H, F, Cl, Br, I, ═O, OH, NH₂, C₁₋₄ alkyl or C₁₋₄ alkoxy;
in certain embodiments, R^{k12} and R^{k13} are each independently selected from H, C₁₋₄ alkyl or C₃₋₆ cycloalkyl, wherein the alkyl or cycloalkyl is optionally further substituted with 0 to 4 (such as 0, 1, 2, 3 or 4) substituents selected from H, F, Cl, Br, I, ═O, OH, NH₂, C₁₋₄ alkyl or C₁₋₄ alkoxy;
in certain embodiments, R^{k12} and R^{k13} are each independently selected from H, methyl, ethyl, isopropyl, propyl, cyclopropyl or cyclobutyl, wherein the methyl, ethyl, isopropyl, propyl, cyclopropyl or cyclobutyl is optionally further substituted with 0 to 4 (such as 0, 1, 2, 3 or 4) substituents selected from H, F, Cl, Br, I, ═O, OH, NH₂, C₁₋₄ alkyl or C₁₋₄ alkoxy;
in certain embodiments, R^{k14} is selected from 5- to 6-membered heteroaryl, wherein the heteroaryl is optionally further substituted with 0 to 4 (such as 0, 1, 2, 3 or 4) substituents selected from H, F, Cl, Br, I, OH, ═O, CF₃, CN, C₁₋₄ alkyl, halogen-substituted C₁₋₄ alkyl, hydroxyl-substituted C₁₋₄ alkyl, C₁₋₄ alkoxy or C₃₋₆ cycloalkyl, and the heteroaryl contains 1 to 4 (such as 1, 2, 3 or 4) heteroatoms selected from N, O or S;
in certain embodiments, K is selected from one of the structural fragments shown in Table K-a;
in certain embodiments, K is selected from one of the following structural fragments:

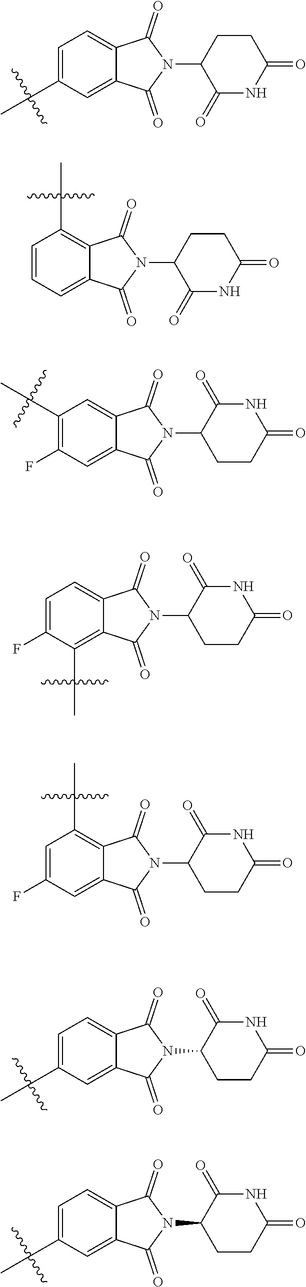

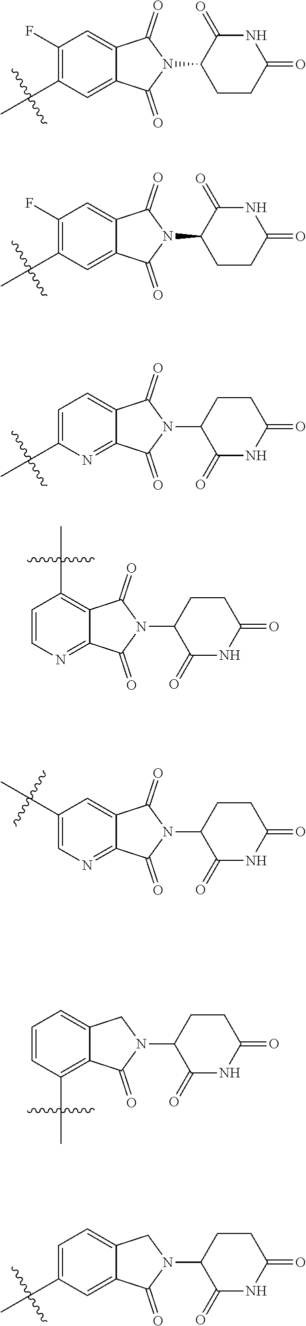

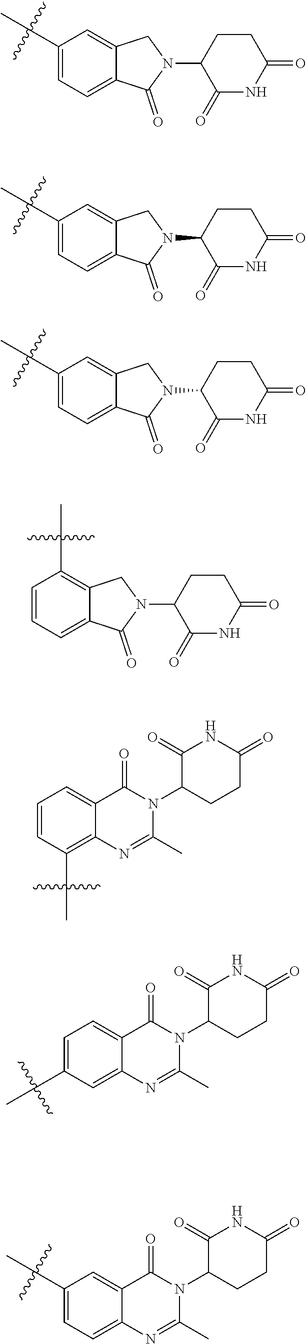

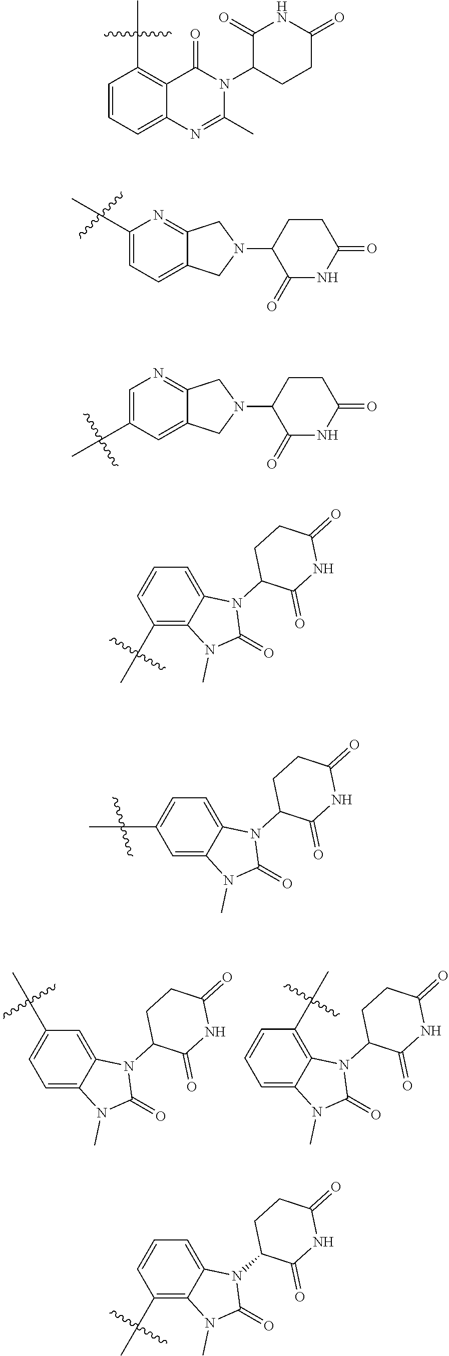

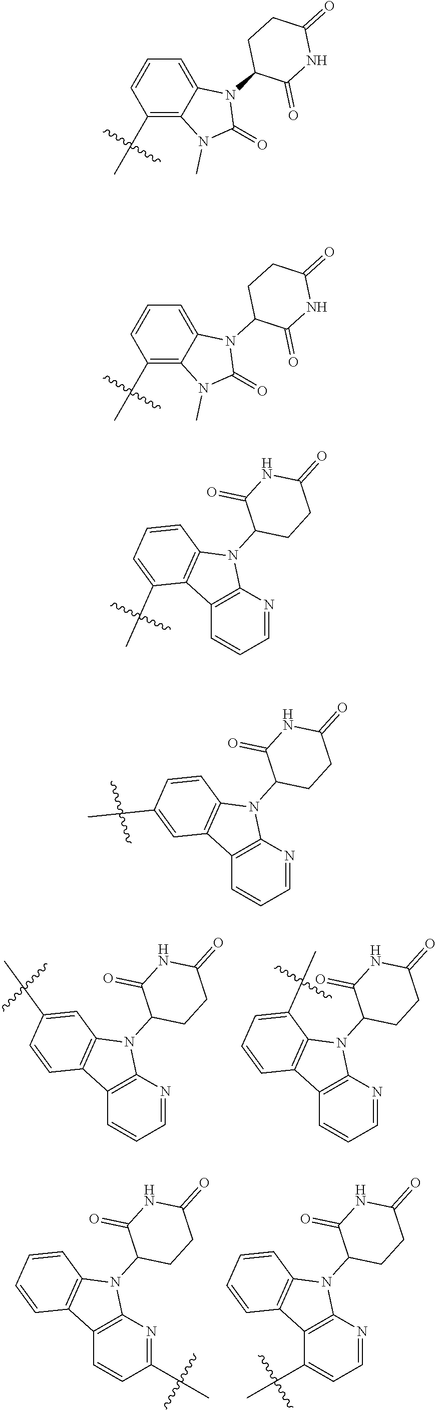

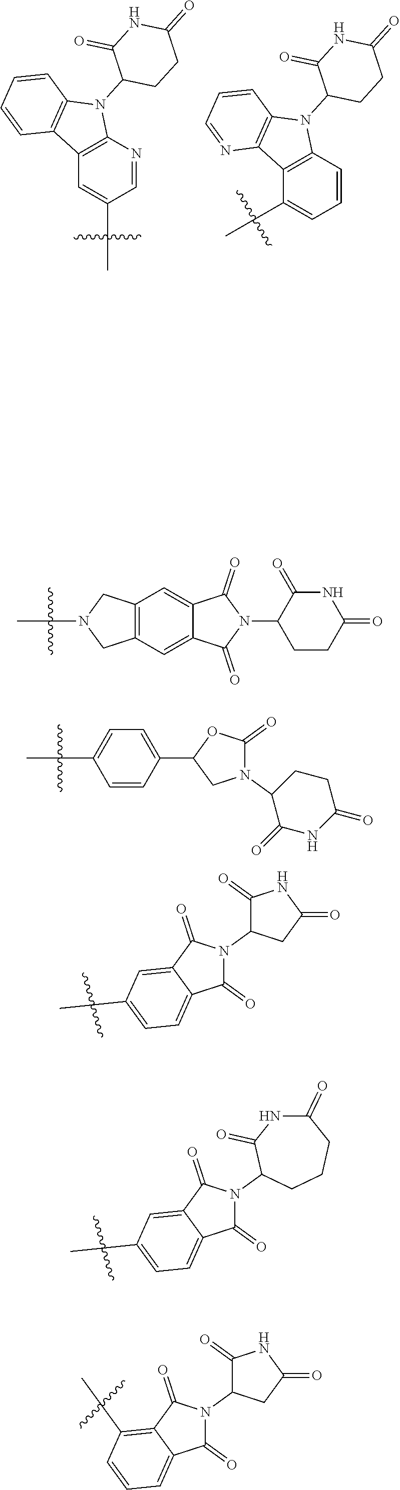

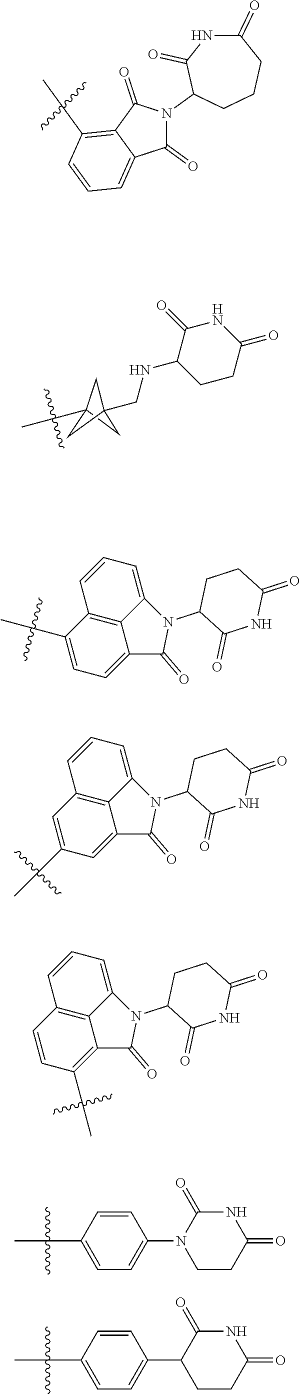

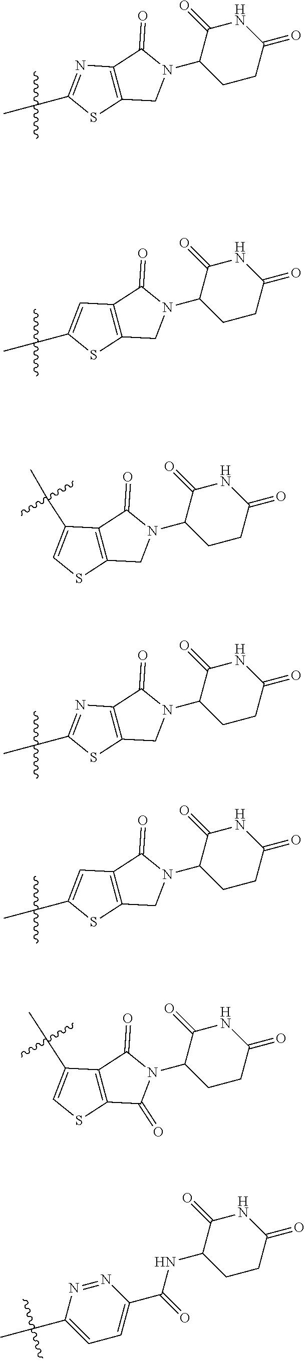

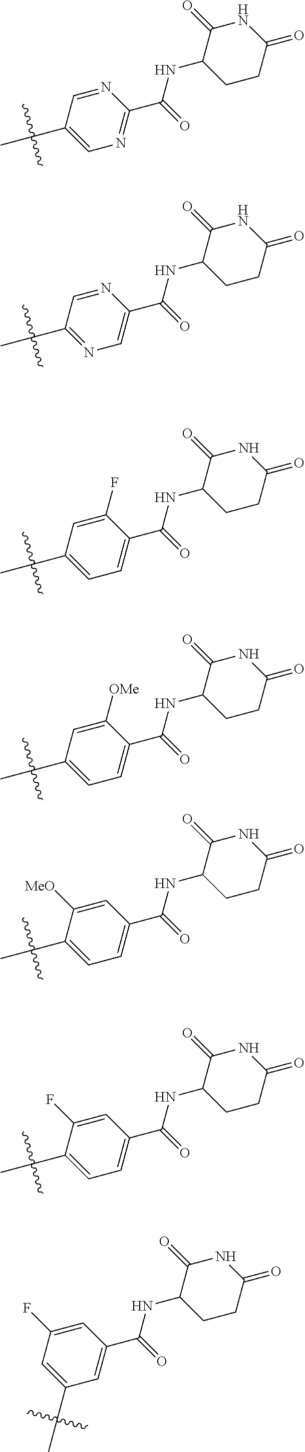

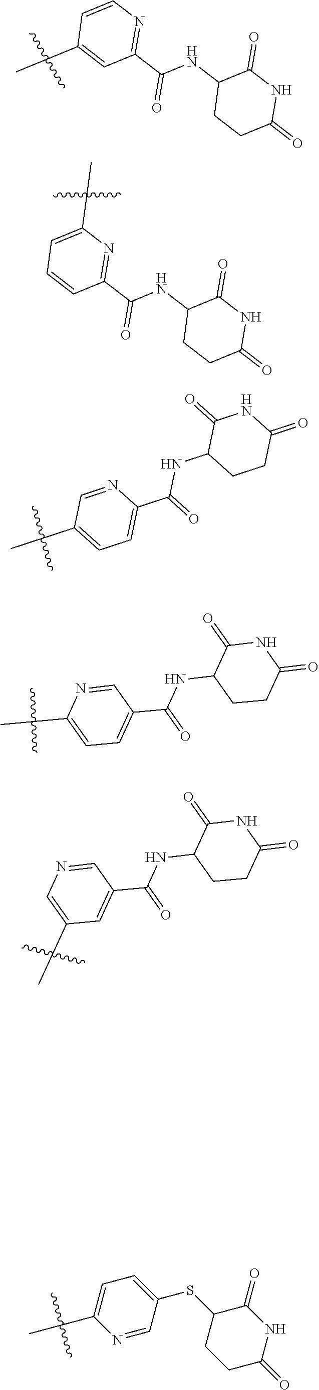

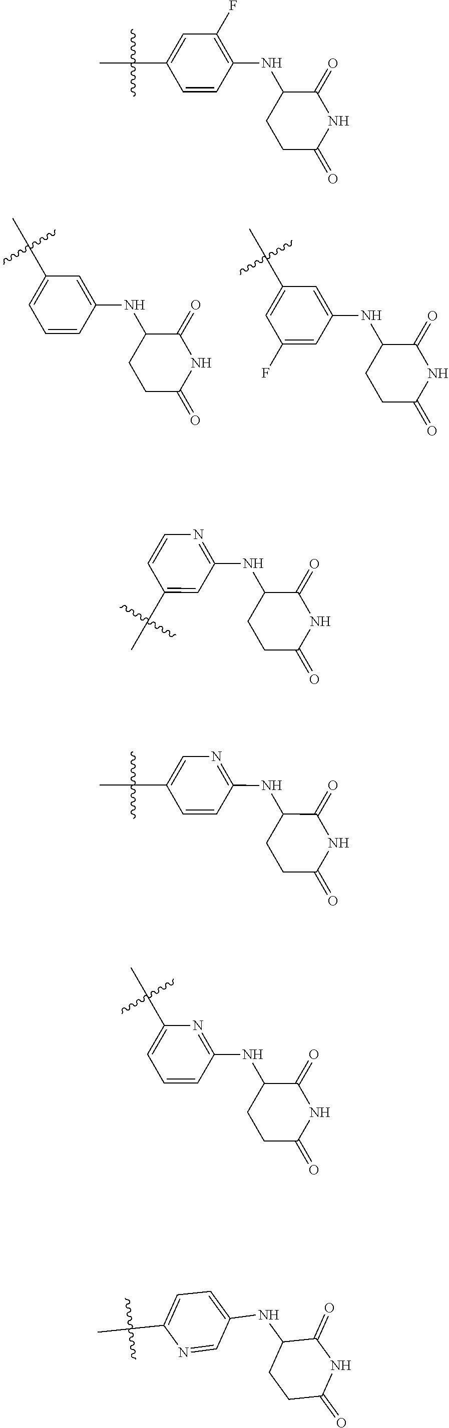

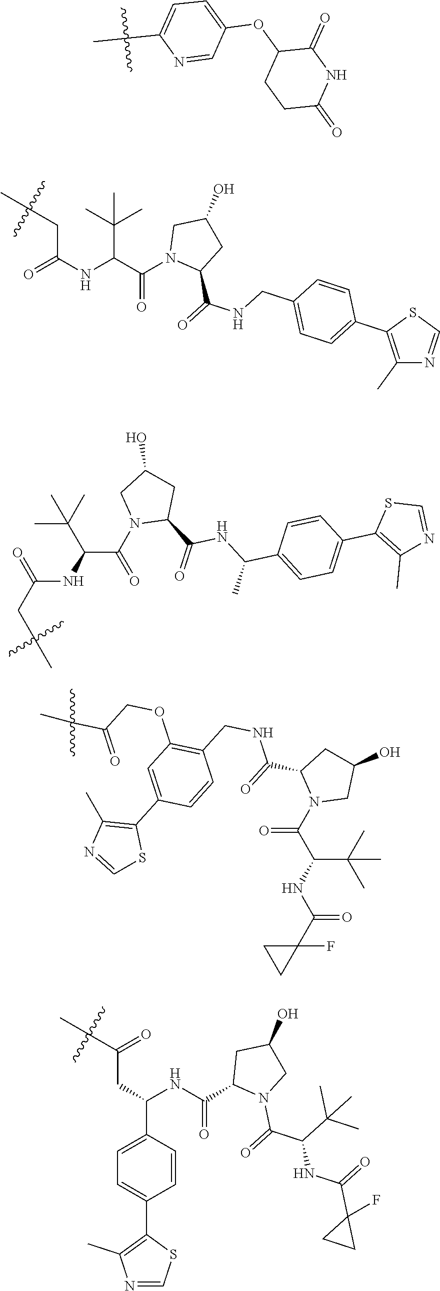

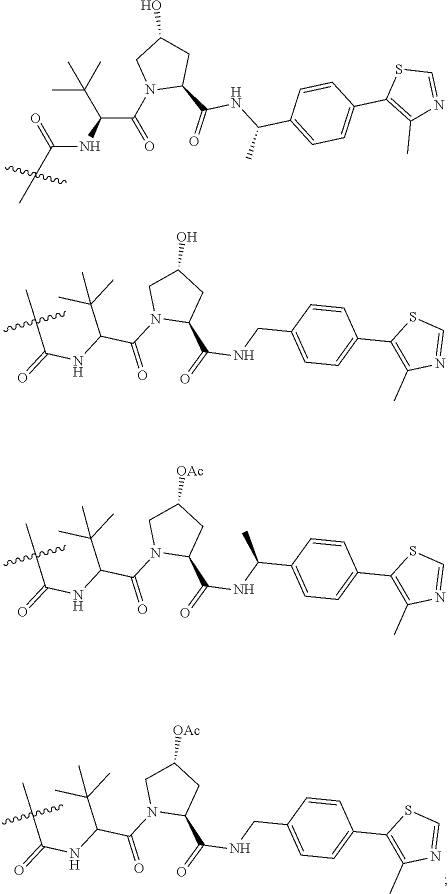 [SMILES: *C(=O)N[C@H](C(=O)N1C[C@H](O)C[C@H]1C(=O)N[C@@H](C)c1ccc(-c2scnc2C)cc1)C(C)(C)C ; *C(=O)NC(C(=O)N1C[C@H](O)C[C@H]1C(=O)NCc1ccc(-c2scnc2C)cc1)C(C)(C)C ; *C(=O)NC(C(=O)N1C[C@H](OC(C)=O)C[C@H]1C(=O)N[C@@H](C)c1ccc(-c2scnc2C)cc1)C(C)(C)C ; *C(=O)NC(C(=O)N1C[C@H](OC(C)=O)C[C@H]1C(=O)NCc1ccc(-c2scnc2C)cc1)C(C)(C)C]

in certain embodiments, K is selected from

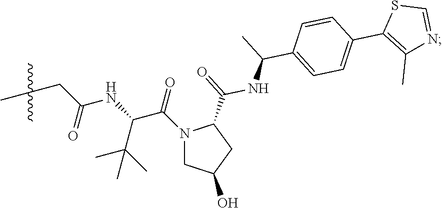 [SMILES: *CC(=O)N[C@H](C(=O)N1C[C@H](O)C[C@H]1C(=O)N[C@@H](C)c1ccc(-c2scnc2C)cc1)C(C)(C)C]

in certain embodiments, B is selected from

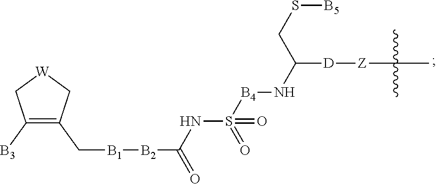

in certain embodiments, B is selected from

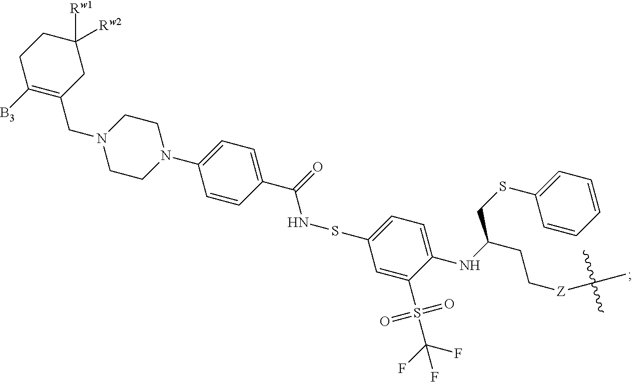

in certain embodiments, B is selected from

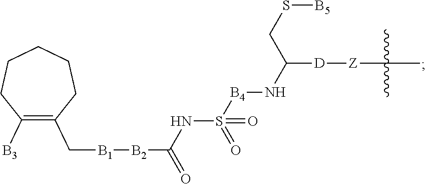

in some embodiments,

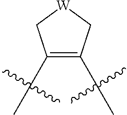

is selected from

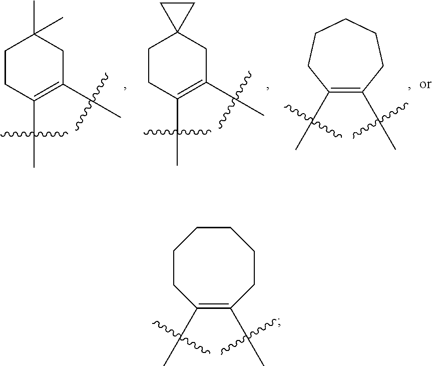

in certain embodiments, W is selected from W₁ or W₂;
in certain embodiments, W₁ is selected from —CR^{w1}R^{w2}—, —(CR^{w1}R^{w2})₃—, —(CR^{w1}R^{w2})₄—, —CH₂CR^{w3}R^{w4}—, —CR^{w3}R^{w4}CH₂—, —CR^{w1}R^{w2}O—, —OCR^{w1}R^{w2}, —CR^{w1}R^{w2}NR^{w5}—, or —NRW⁵CR^{w1}R^{w2}—;
in certain embodiments, W₂ is selected from —(CR^{w1}R^{w2})₂—;
in certain embodiments, D is selected from C₁₋₄ alkylene;
in certain embodiments, D is selected from ethylene;
in certain embodiments,

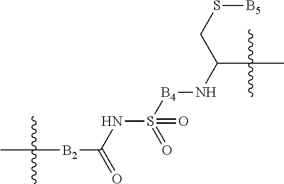

is selected from

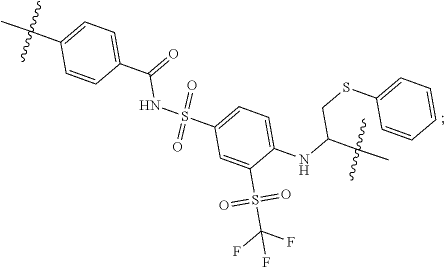 [SMILES: *c1ccc(C(=O)NS(=O)(=O)c2ccc(NC(*)CSc3ccccc3)c(S(=O)(=O)C(F)(F)F)c2)cc1]

in certain embodiments, B₁ and Z are each independently selected from a 4- to 7-membered mono-heterocyclic ring, a 5- to 12-membered fused-heterocyclic ring, a 6- to 12-membered spiro-heterocyclic ring, or a 7- to 12-membered bridged-heterocyclic ring, the B₁ is optionally further substituted with 0 to 4 R^{B1}, and the Z is optionally further substituted with 0 to 4 R^{Q}, wherein the fused-heterocyclic ring, spiro-heterocyclic ring, or bridged-heterocyclic ring contains 1 to 3 heteroatoms selected from O, S or N, and is optionally further substituted with 0, 1 or 2 ═O when the heteroatom is selected from S;
in certain embodiments, B₁ and Z are each independently selected from azetidinyl, azacyclopentyl, piperazinyl, piperidyl, azacyclohexenyl, azepanyl, 1,4-diazepanyl, cyclobutyl-fused-azetidinyl, cyclobutyl-fused-azacyclopentyl, cyclobutyl-fused-azacyclohexyl, cyclobutyl-fused-piperidyl, cyclopentyl-fused-azetidinyl, cyclopentyl-fused-azacyclopentyl, cyclopentyl-fused-azacyclohexyl, cyclopentyl-fused-piperidyl, cyclohexyl-fused-azetidinyl, cyclohexyl-fused-azacyclopentyl, cyclohexyl-fused-azacyclohexyl, cyclohexyl-fused-piperidyl, azetidinyl-fused-azetidinyl, azacyclopentyl-fused-azetidinyl, azacyclopentyl-fused-azacyclopentyl, azacyclopentyl-fused-azacyclohexyl, azacyclopentyl-fused-piperidyl, azacyclohexyl-fused-azetidinyl, azacyclohexyl-fused-azacyclopentyl, azacyclohexyl-fused-azacyclohexyl, azacyclohexyl-fused-piperidyl, cyclobutyl-spiro-azetidinyl, cyclobutyl-spiro-azacyclopentyl, cyclobutyl-spiro-azacyclohexyl, cyclopentyl-spiro-azetidinyl, cyclopentyl-spiro-azacyclopentyl, cyclopentyl-spiro-azacyclohexyl, cyclohexyl-spiro-azetidinyl, cyclohexyl-spiro-azacyclopentyl, azetidinyl-spiro-azetidinyl, azetidinyl-spiro-azacyclopentyl, azetidinyl-spiro-azacyclohexyl, azacyclopentyl-spiro-azetidinyl, azacyclopentyl-spiro-azacyclopentyl, azacyclopentyl-spiro-azacyclohexyl, azacyclohexyl-spiro-azetidinyl, azacyclohexyl-spiro-azacyclopentyl, azacyclohexyl-spiro-azacyclohexyl, cyclobutyl-spiro-piperidyl, cyclopentyl-spiro-piperidyl, cyclohexyl-spiro-piperidyl, azetidinyl-spiro-piperidyl, azacyclopentyl-spiro-piperidyl, azacyclohexyl-spiro-piperidyl,

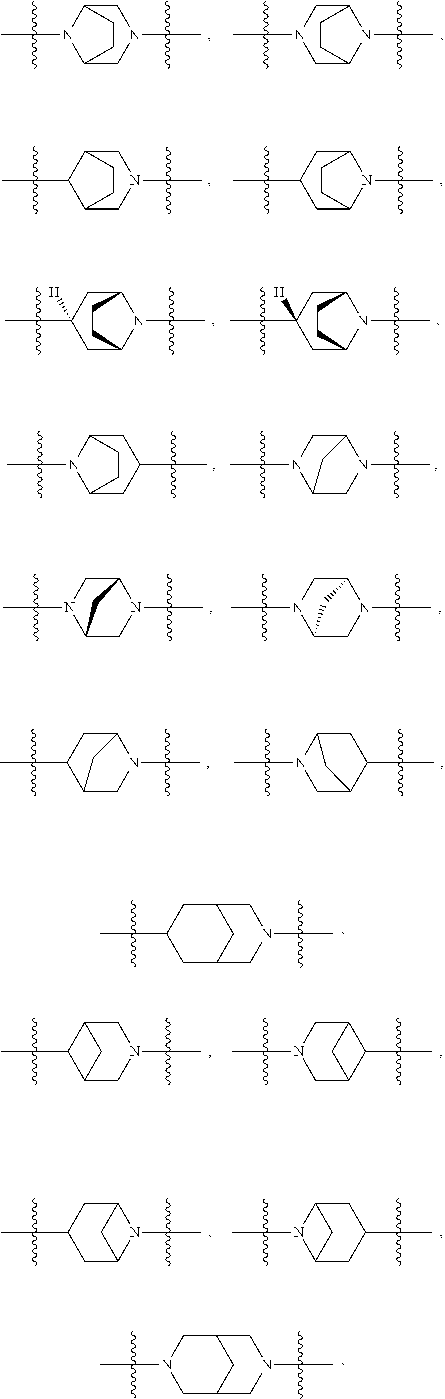

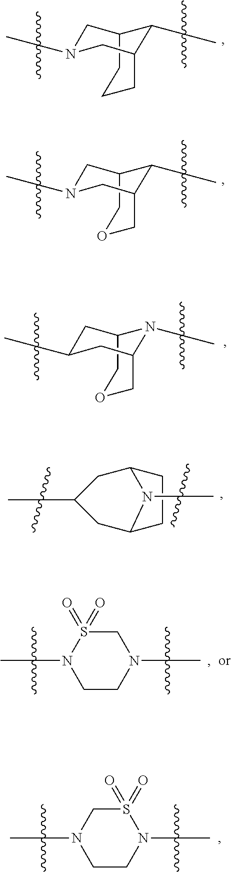 [SMILES: *C1C2CCCC1CN(*)C2 ; *C1C2COCC1CN(*)C2 ; *C1CC2COCC(C1)N2* ; *C1CC2CCC(C1)N2* ; *N1CCN(*)S(=O)(=O)C1 ; *N1CCN(*)S(=O)(=O)C1]

the B₁ is optionally further substituted with 0 to 4 R^{B1}, and the Z is optionally further substituted with 0 to 4 R^{Q};
in certain embodiments, B₁ and Z are each independently selected from

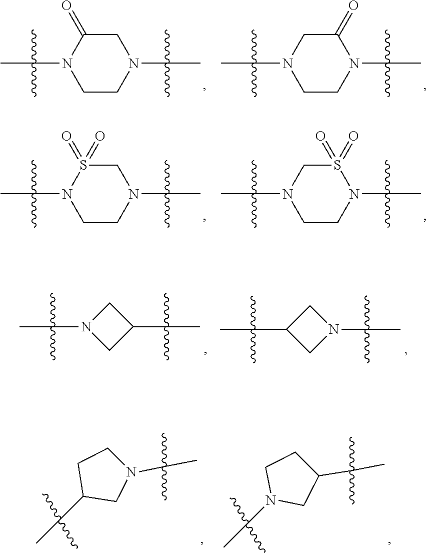

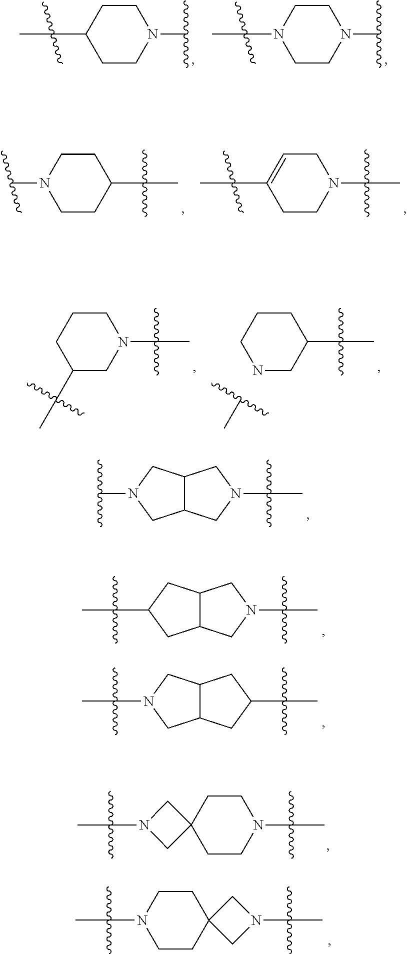

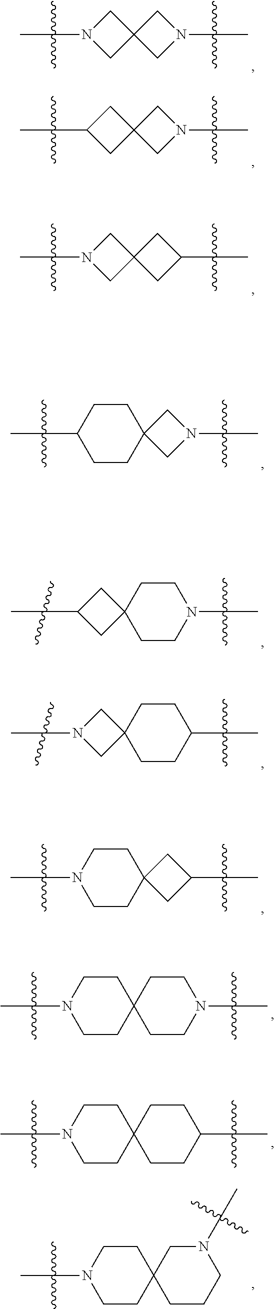

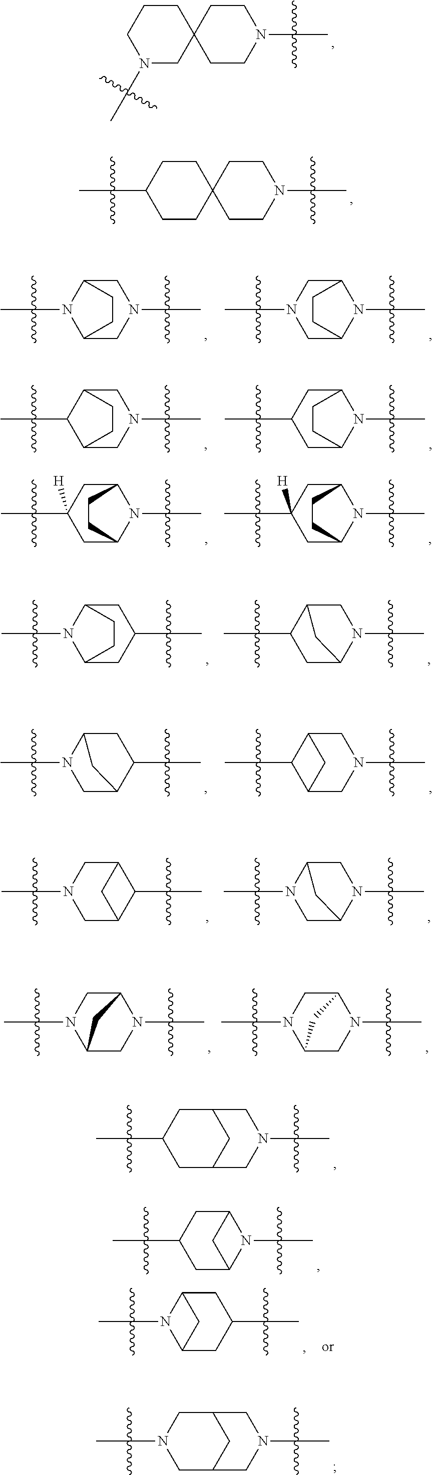

in certain embodiments, B₁ and Z are each independently selected from

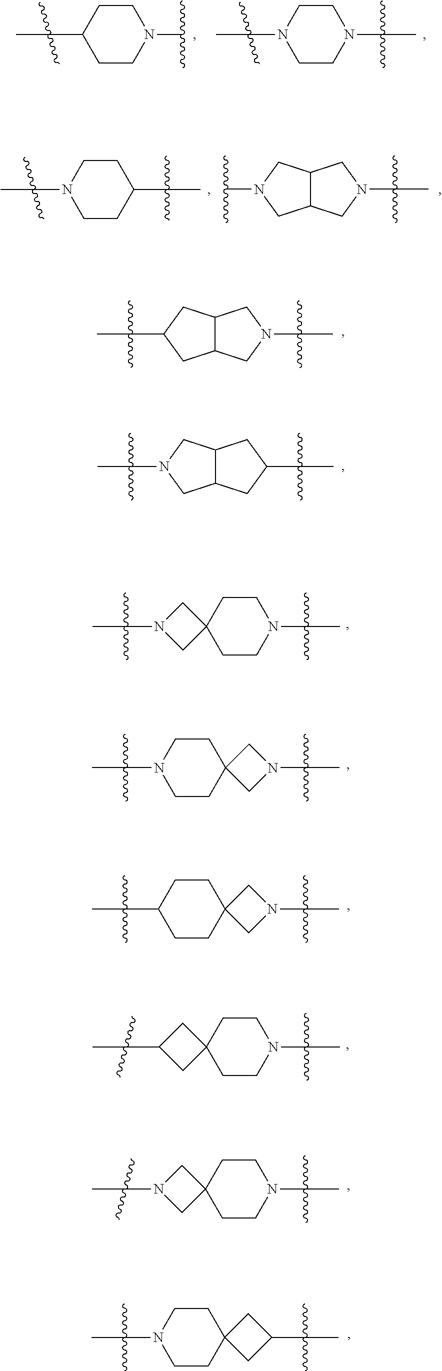

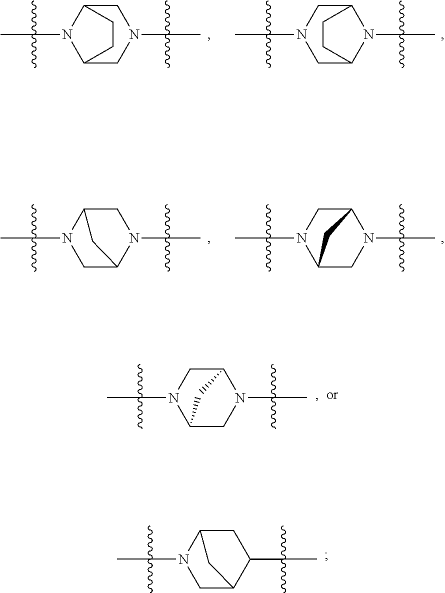 [SMILES: *N1CC2CCC(C1)N2* ; *N1CC2CCC(C1)N2* ; *N1CC2CC1CN2* ; *N1C[C@H]2C[C@@H]1CN2* ; *N1C[C@@H]2C[C@H]1CN2* ; *C1CC2CC1CN2*]

in certain embodiments, B₂, B₃, B₄, and B₅ are each independently selected from C₆₋₁₀ aryl or 5- to 10-membered heteroaryl, the B₂ is optionally further substituted with 0 to 4 R^{B2}, the B₃ is optionally further substituted with 0 to 5 R^{B3}, the B₄ is optionally further substituted with 0 to 4 R^{B4}, and the B₅ is optionally further substituted with 0 to 5 R^{B5}, wherein the heteroaryl contains 1 to 3 heteroatoms selected from O, S or N;
in certain embodiments, B₂ and B₄ are each independently selected from phenyl or 5- to 6-membered heteroaryl, B₃ and B₅ are each independently selected from phenyl, naphthyl, 5- to 6-membered heteroaryl or benzo 5- to 6-membered heteroaryl, the B₂ is optionally further substituted with 0 to 4 R^{B2}, the B₃ is optionally further substituted with 0 to 5 R^{B3}, the B₄ is optionally further substituted with 0 to 4 R^{B4}, and the B₅ is optionally further substituted with 0 to 5 R^{B5}, wherein the heteroaryl contains 1 to 3 heteroatoms selected from O, S or N;
in certain embodiments, B₃ is selected from phenyl, naphthyl, 5- to 6-membered heteroaryl, or benzo 5- to 6-membered heteroaryl and the B₃ is optionally further substituted with 0 to 5 R^{B3};
in certain embodiments, B₃ is selected from

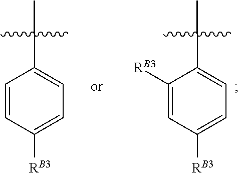

in certain embodiments, B₃ is selected from

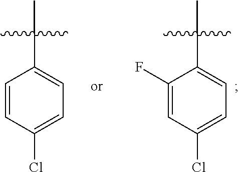 [SMILES: *c1ccc(Cl)cc1 ; *c1ccc(Cl)cc1F]

in certain embodiments, B₂ and B₄ are each independently selected from phenyl, thienyl, furyl, pyrrolyl, imidazolyl, pyrazolyl, oxazolyl, thiazolyl, pyridyl, pyrimidyl, pyrazinyl, or pyridazinyl, B₃ and B₅ are each independently selected from phenyl, naphthyl, thienyl, furyl, pyrrolyl, imidazolyl, pyridyl, pyrimidyl, pyrazinyl, pyridazinyl, benzothienyl, benzofuryl, benzopyrrolyl, or benzoimidazolyl, the B₂ is optionally further substituted with 0 to 4 R^{B2}, the B₃ is optionally further substituted with 0 to 5 R^{B3}, the B₄ is optionally further substituted with 0 to 4 R^{B4}, and the B₅ is optionally further substituted with 0 to 5 RBs;
in certain embodiments, R^{B1}, R^{Q}, R^{B2}, R^{B3}, and R^{B5} are each independently selected from halogen, oxo, OH, CN, C₁₋₄ alkyl or C₁₋₄ alkoxy, wherein the alkyl or alkoxy is optionally further substituted with 0 to 4 substituents selected from H, halogen, OH, CN, or C₁₋₄ alkyl;
in certain embodiments, R^{B1}, R^{Q}, R^{B2}, R^{B3} and R^{B5} are each independently selected from F, Cl, Br, I, oxo, OH, CN, methyl, ethyl, methoxy or ethoxy, wherein the methyl, ethyl, methoxy or ethoxy is optionally further substituted with 0 to 4 substituents selected from H, halogen, OH, CN or C₁₋₄ alkyl;
in certain embodiments, each R^{B4} is independently selected from —SO₂—C₁₋₄ alkyl, nitro, halogen, CN, OH, C₁₋₄ alkyl or C₁₋₄ alkoxy, wherein the alkyl or alkoxy is optionally further substituted with 0 to 4 substituents selected from H, halogen, OH, CN or C₁₋₄ alkyl;
in certain embodiments, each R^{B4} is independently selected from —SO₂-methyl, —SO₂-ethyl, nitro, F, Cl, Br, I, OH, CN, methyl, ethyl, methoxy or ethoxy, wherein the methyl, ethyl, methoxy or ethoxy is optionally further substituted with 0 to 4 substituents selected from H, halogen, OH, CN or C₁₋₄ alkyl;
in certain embodiments, each R^{B4} is independently selected from —SO₂—CF₃ or nitro;
in certain embodiments, R^{w1}, R^{w2} and R^{w5} are each independently selected from H, halogen, CN, OH, C₁₋₄ alkyl or C₁₋₄ alkoxy, wherein the alkyl or alkoxy is optionally further substituted with 0 to 4 substituents selected from H, halogen, OH, CN or C₁₋₄ alkyl;
in certain embodiments, R^{w1}, R^{w2} and R^{w5} are each independently selected from H, F, Cl, Br, I, OH, CN, methyl, ethyl, methoxy or ethoxy, wherein the methyl, ethyl, methoxy or ethoxy is optionally further substituted with 0 to 4 substituents selected from H, halogen, OH, CN or C₁₋₄ alkyl;
in certain embodiments, R^{w1} and R^{w2} are directly connected to form C₃₋₆ carbocycle or 3- to 6-membered heterocycle, wherein the carbocycle or heterocycle is optionally further substituted with 0 to 4 substituents selected from H, halogen, OH, CN or C₁₋₄ alkyl, and the heterocycle contains 1 to 3 heteroatoms selected from O, S or N;
in certain embodiments, R^{w1} and R^{w2} are directly connected to form cyclopropyl, cyclobutyl, cyclopentyl, cyclohexyl, azetidinyl, azacyclopentyl, azacyclohexyl, oxetanyl, oxacyclopentyl, or oxacyclohexyl, wherein the cyclopropyl, cyclobutyl, cyclopentyl, cyclohexyl, azetidinyl, azacyclopentyl, azacyclohexyl, oxetanyl, oxacyclopentyl, or oxacyclohexyl is optionally further substituted with 0 to 4 substituents selected from H, halogen, OH, CN or C₁₋₄ alkyl;
in certain embodiments, R^{w1} and R^{w2} are selected from methyl, or R^{w1} and R^{w2} are directly connected to form, together with the carbon atom to which they are attached, cyclopropyl;
in certain embodiments, R^{w3} and R^{w4} are directly connected to form C₃₋₆ carbocycle or 3- to 6-membered heterocycle, wherein the carbocycle or heterocycle is optionally further substituted with 0 to 4 substituents selected from H, halogen, OH, CN or C₁₋₄ alkyl, and the heterocycle contains 1 to 3 heteroatoms selected from O, S or N;
in certain embodiments, R^{w3} and R^{w4} are directly connected to form cyclopropyl, cyclobutyl, cyclopentyl, cyclohexyl, azetidinyl, azacyclopentyl, azacyclohexyl, oxetanyl, oxacyclopentyl, or oxacyclohexyl, wherein the cyclopropyl, cyclobutyl, cyclopentyl, cyclohexyl, azetidinyl, azacyclopentyl, azacyclohexyl, oxetanyl, oxacyclopentyl, or oxacyclohexyl is optionally further substituted with 0 to 4 substituents selected from H, halogen, OH, CN or C₁₋₄ alkyl;
in certain embodiments, R^{B1} and R^{B2} are directly connected to form C₅₋₇ carbocycle or 5- to 7-membered heterocycle, wherein the carbocycle or heterocycle is optionally further substituted with 0 to 3 R^{c}, and the heterocycle contains 1 to 3 heteroatoms selected from O, S or N;
in certain embodiments, each R^{c} is independently selected from halogen, OH, CN, ═O, C₁₋₄ alkyl, C₁₋₄ alkoxy, C₃₋₆ carbocycle or 3- to 7-membered heterocycle, wherein the alkyl, alkoxy, carbocycle or heterocycle is optionally further substituted with 0 to 4 substituents selected from H, halogen, OH, CN, C₁₋₄ alkyl, C₁₋₄ alkoxy, C₃₋₆ cycloalkyl or 3- to 7-membered heterocycloalkyl, and the heterocycle or heterocycloalkyl contains 1 to 3 heteroatoms selected from O, S or N,
in certain embodiments, each R^{c} is independently selected from F, Cl, Br, I, OH, CN, ═O, methyl, ethyl, methoxy, ethoxy, cyclopropyl, cyclobutyl, cyclopentyl, cyclohexyl, oxetanyl, oxacyclopentyl, oxacyclohexyl, azetidinyl, azacyclopentyl, azacyclohexyl, morpholinyl, or piperazinyl, wherein the methyl, ethyl, methoxy, ethoxy, cyclopropyl, cyclobutyl, cyclopentyl, cyclohexyl, oxetanyl, oxacyclopentyl, oxacyclohexyl, azetidinyl, azacyclopentyl, azacyclohexyl, morpholinyl, or piperazinyl is optionally further substituted with 0 to 4 substituents selected from H, halogen, OH, CN, C₁₋₄ alkyl, C₁₋₄ alkoxy, C₃₋₆ cycloalkyl, or 3- to 7-membered heterocycloalkyl, and the heterocycloalkyl contains 1 to 3 heteroatoms selected from O, S or N;
in certain embodiments, Z is selected from

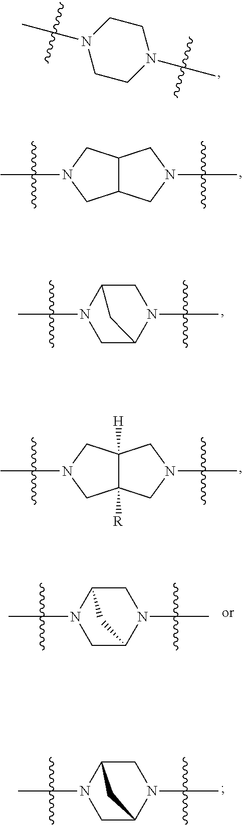

in certain embodiments, when Z is selected from

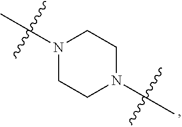 [SMILES: *N1CCN(*)CC1]

and R^{w1} and R^{w2} are selected from methyl, B₃ is selected from

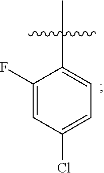 [SMILES: *c1ccc(Cl)cc1F]

in some embodiments, the compound of general formula (I) is defined as follows:
when W₂ is selected from —(CR^{w1}R^{w2})₂—, and R^{w1} and R^{w2} are each independently selected from F, methyl or methoxy, B at least satisfies any one of the following conditions:
1) B₁ is not piperazine;
2) Z is not piperazine, piperidine,

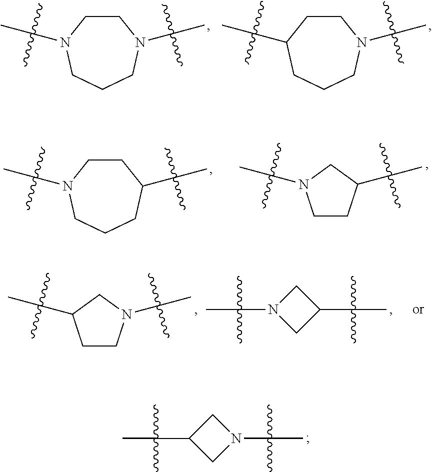

3) when B₃ is selected from phenyl, the phenyl is substituted with 1 to 4 R^{B3} and at least one R^{B3} is not halogen, methyl or trifluoromethyl;
4) when B₂ is selected from phenyl, the phenyl is substituted with 1 to 4 R^{B2};
5) R^{B1} and R^{B2} are directly connected to form C₅₋₇ carbocycle or 5- to 7-membered heterocycle, wherein the carbocycle or heterocycle is optionally further substituted with 0 to 3 R^{c}, and the heterocycle contains 1 to 3 heteroatoms selected from O, S or N;
6) when B₅ is selected from phenyl, the phenyl is substituted with 1 to 4 R^{B5};
in some embodiments, the compound of general formula (I) is defined as follows:
when W₂ is selected from —(CR^{w1}R^{w2})₂—, and R^{w1} and R^{w2} are each independently selected from F, methyl or methoxy, B at least satisfies any one of the following conditions:
1) B₁ is not piperazine;
2) Z is not piperazine, piperidine,

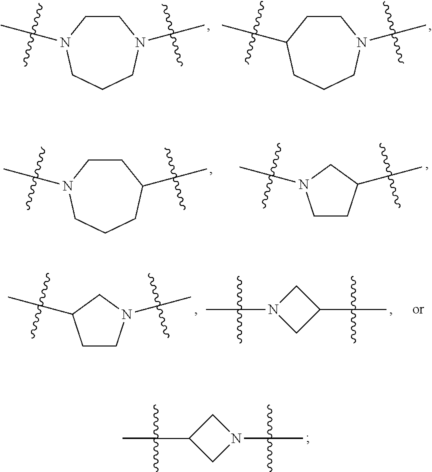

3) B₃ is selected from phenyl substituted with 1 R^{B3}, when R^{B3} is at the para-position of the phenyl, R^{B3} is not halogen, methyl or trifluoromethyl;
4) when B₂ is selected from phenyl, the phenyl is substituted with 1 to 4 R^{B2};
5) R^{B1} and R^{B2} are directly connected to form C₅₋₇ carbocycle or 5- to 7-membered heterocycle, wherein the carbocycle or heterocycle is optionally further substituted with 0 to 3 R^{c}, and the heterocycle contains 1 to 3 heteroatoms selected from O, S or N;
6) when B₅ is selected from phenyl, the phenyl is substituted with 1 to 4 R^{B5};
optionally, 1 to 20 H of the compound of general formula (I) are replaced by 1 to 20 deuterium;
in certain embodiments, B is selected from one of the structural fragments shown in Table B-a;
in certain embodiments, each q is independently selected from 0, 1, 2, 3, 4, 5 or 6;
in certain embodiments, each q is independently selected from 0, 1, 2, 3 or 4;
in certain embodiments, each q is independently selected from 0, 1, 2 or 3;
in certain embodiments, each q is independently selected from 0, 1 or 2;
in certain embodiments, n1, n2 and n3 are each independently selected from 0, 1, 2 or 3;
in certain embodiments, each p1 or p2 is independently selected from 0, 1, 2, 3, 4 or 5;
in certain embodiments, each p1 or p2 is independently selected from 0, 1 or 2.

As a first embodiment of the present invention, provided is the above-mentioned compound of general formula (I) or the stereoisomer, deuterated compound, solvate, prodrug, metabolite, pharmaceutically acceptable salt or co-crystal thereof, wherein
L is selected from a bond or —C₁₋₅₀ hydrocarbyl-, wherein the hydrocarbyl has 0 to 20 methylene units optionally further replaced by -Ak- or -Cy-;
each -Ak- is independently selected from —(CH₂)_{q}—, —(CH₂)_{q}—O—, —O—(CH₂)_{q}—, —(CH₂)_{q}—NR^{L}—, —NR^{L}—(CH₂)_{q}—, —(CH₂)_{q}—NR^{L}C(═O)—, —NR^{L}(CH₂)_{q}C(═O)—, —(CH₂)_{q}—C(═O)NR^{L}—, —C(═O)—, —C(═O)—(CH₂)_{q}—NR^{L}—, —(C≡C)_{q}—, —CH═CH—, —Si(R^{L})₂—, —Si(OH)(R^{L})—, —Si(OH)₂—, —P(═O)(OR^{L})—, —P(═O)(R^{L})—, —S—, —S(═O)—, —S(═O)₂— or a bond, wherein the —CH₂— is optionally further substituted with 0 to 2 substituents selected from H, halogen, OH, CN, NH₂, C₁₋₆ alkyl, C₁₋₆ alkoxy, halogen-substituted C₁₋₆ alkyl, hydroxyl-substituted C₁₋₆ alkyl, or cyano-substituted C₁₋₆ alkyl;
each q is independently selected from 0, 1, 2, 3, 4, 5 or 6;
each R^{L} is independently selected from H, C₁₋₆ alkyl, 3- to 7-membered heterocyclyl, 3- to 7-membered cycloalkyl, phenyl or 5- to 6-membered heteroaryl;
each -Cy- is independently selected from a bond, a 4- to 8-membered mono-heterocyclic ring, a 4- to 10-membered fused-heterocyclic ring, a 5- to 12-membered spiro-heterocyclic ring, a 7- to 10-membered bridged-heterocyclic ring, 3- to 7-membered monocycloalkyl, 4- to 10-membered fused cycloalkyl, 5- to 12-membered spiro cycloalkyl, 7- to 10-membered bridged cycloalkyl, 5- to 10-membered heteroaryl or 6- to 10-membered aryl, wherein the aryl, heteroaryl, cycloalkyl, mono-heterocyclic ring, fused-heterocyclic ring, spiro-heterocyclic ring or bridged-heterocyclic ring is optionally further substituted with 0 to 4 substituents selected from H, F, Cl, Br, I, OH, COOH, CN, NH₂, ═O, C₁₋₄ alkyl, halogen-substituted C₁₋₄ alkyl, hydroxyl-substituted C₁₋₄ alkyl or C₁₋₄ alkoxy, and the heteroaryl, mono-heterocyclic ring, fused-heterocyclic ring, spiro-heterocyclic ring or bridged-heterocyclic ring contains 1 to 4 heteroatoms selected from O, S or N, and is optionally further substituted with 0, 1 or 2 ═O when the heteroatom is selected from S;
B is selected from

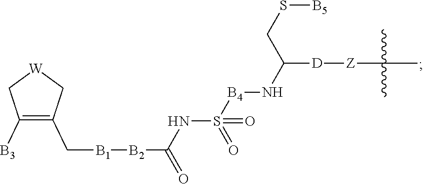

W is selected from W₁ or W₂;
W₁ is selected from —CR^{w1}R^{w2}—, —(CR^{w1}R^{w2})₃—, —(CR^{w1}R^{w2})₄—, —CH₂CR^{w3}R^{w4}—, —CR^{w3}R^{w4}CH₂—, —CR^{w1}R^{w2}O—, —OCR^{w1}R^{w2}—, —CR^{w1}R^{w2}NR^{w5}—, or —NR^{w5}CR^{w1}R^{w2}—;
W₂ is selected from —(CR^{w1}R^{w2})₂—;
D is selected from C₁₋₄ alkylene;
B₁ and Z are each independently selected from a 4- to 7-membered mono-heterocyclic ring, a 5- to 12-membered fused-heterocyclic ring, a 6- to 12-membered spiro-heterocyclic ring, or a 7- to 12-membered bridged-heterocyclic ring, the B₁ is optionally further substituted with 0 to 4 R^{B1}, and the Z is optionally further substituted with 0 to 4 R^{Q}, wherein the fused-heterocyclic ring, spiro-heterocyclic ring, or bridged-heterocyclic ring contains 1 to 3 heteroatoms selected from O, S or N, and is optionally further substituted with 0, 1 or 2 ═O when the heteroatom is selected from S;
B₂, B₃, B₄, and B₅ are each independently selected from C₆₋₁₀ aryl or 5- to 10-membered heteroaryl, the B₂ is optionally further substituted with 0 to 4 R^{B2}, the B₃ is optionally further substituted with 0 to 5 R^{B3}, the B₄ is optionally further substituted with 0 to 4 R^{B4}, and the B₅ is optionally further substituted with 0 to 5 R^{B5}, wherein the heteroaryl contains 1 to 3 heteroatoms selected from O, S or N;
R^{B1}, R^{Q}, R^{B2}, R^{B3}, and R^{B5} are each independently selected from halogen, oxo, OH, CN, C₁₋₄ alkyl or C₁₋₄ alkoxy, wherein the alkyl or alkoxy is optionally further substituted with 0 to 4 substituents selected from H, halogen, OH, CN, or C₁₋₄ alkyl;
each R^{B4} is independently selected from —SO₂—C₁₋₄ alkyl, nitro, halogen, CN, OH, C₁₋₄ alkyl or C₁₋₄ alkoxy, wherein the alkyl or alkoxy is optionally further substituted with 0 to 4 substituents selected from H, halogen, OH, CN or C₁₋₄ alkyl;
R^{w1}, R^{w2} and R^{w5} are each independently selected from H, halogen, CN, OH, C₁₋₄ alkyl or C₁₋₄ alkoxy, wherein the alkyl or alkoxy is optionally further substituted with 0 to 4 substituents selected from H, halogen, OH, CN or C₁₋₄ alkyl;
alternatively, R^{w1} and R^{w2} are directly connected to form C₃₋₆ carbocycle or 3- to 6-membered heterocycle, wherein the carbocycle or heterocycle is optionally further substituted with 0 to 4 substituents selected from H, halogen, OH, CN or C₁₋₄ alkyl, and the heterocycle contains 1 to 3 heteroatoms selected from O, S or N;
R^{w3} and R^{w4} are directly connected to form C₃₋₆ carbocycle or 3- to 6-membered heterocycle, wherein the carbocycle or heterocycle is optionally further substituted with 0 to 4 substituents selected from H, halogen, OH, CN or C₁₋₄ alkyl, and the heterocycle contains 1 to 3 heteroatoms selected from O, S or N;
alternatively, R^{B1} and R^{B2} are directly connected to form C₅₋₇ carbocycle or 5- to 7-membered heterocycle, wherein the carbocycle or heterocycle is optionally further substituted with 0 to 3 R^{c}, and the heterocycle contains 1 to 3 heteroatoms selected from O, S or N;
each R^{c} is independently selected from halogen, OH, CN, ═O, C₁₋₄ alkyl, C₁₋₄ alkoxy, C₃₋₆ carbocycle or 3- to 7-membered heterocycle, wherein the alkyl, alkoxy, carbocycle or heterocycle is optionally further substituted with 0 to 4 substituents selected from H, halogen, OH, CN, C₁₋₄ alkyl, C₁₋₄ alkoxy, C₃₋₆ cycloalkyl or 3- to 7-membered heterocycloalkyl, and the heterocycle or heterocycloalkyl contains 1 to 3 heteroatoms selected from O, S or N,
provided that when W₂ is selected from —(CR^{w1}R^{w2})₂—, and R^{w1} and R^{w2} are each independently selected from F, methyl or methoxy, B at least satisfies any one of the following conditions:
1) B₁ is not piperazine;
2) Z is not piperazine, piperidine,

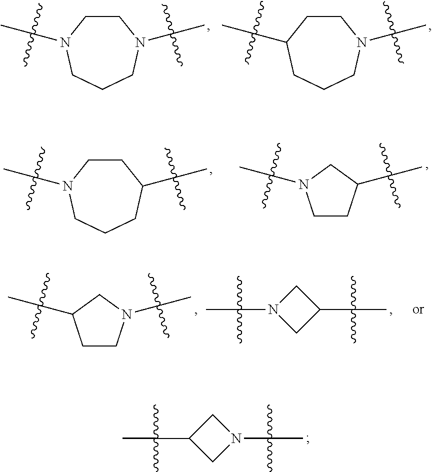

3) B₃ is selected from phenyl substituted with 1 R^{B3}, when R^{B3} is at the para-position of the phenyl, R^{B3} is not halogen, methyl or trifluoromethyl;
4) when B₂ is selected from phenyl, the phenyl is substituted with 1 to 4 R^{B2};
5) R^{B1} and R^{B2} are directly connected to form C₅₋₇ carbocycle or 5- to 7-membered heterocycle, wherein the carbocycle or heterocycle is optionally further substituted with 0 to 3 R^{c}, and the heterocycle contains 1 to 3 heteroatoms selected from O, S or N;
6) when B₅ is selected from phenyl, the phenyl is substituted with 1 to 4 R^{B5};
K is selected from

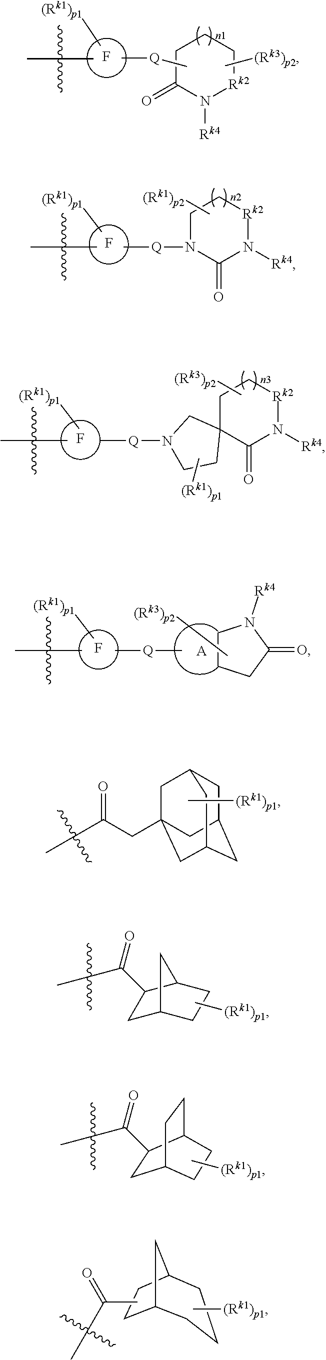

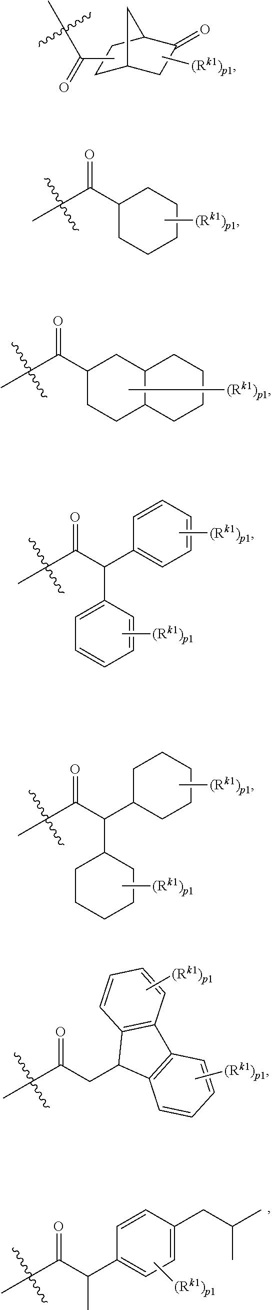

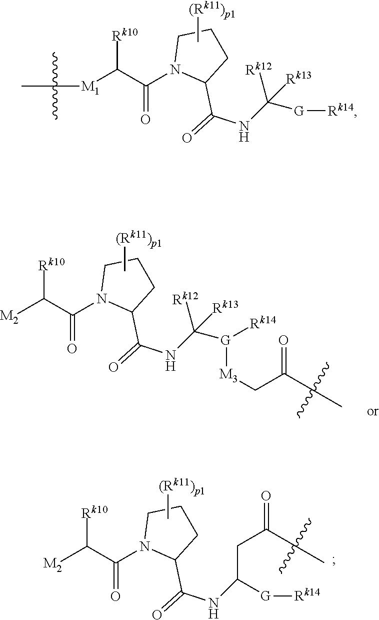

each Q is independently selected from a bond, —O—, —S—, —CH₂—, —NR^{q}—, —CO—, —NR^{q}CO—, —CONR^{q}— or 3- to 12-membered heterocyclyl, wherein the heterocyclyl is optionally further substituted with 0 to 4 substituents selected from H, F, Cl, Br, I, OH, ═O, NH₂, CN, COOH, CONH₂, C₁₋₄ alkyl or C₁₋₄ alkoxy, and the heterocyclyl contains 1 to 4 heteroatoms selected from O, S or N;
R^{q} is selected from H or C₁₋₆ alkyl;
A is selected from C₃₋₁₀ carbocyclyl, C₆₋₁₀ aryl, 3- to 10-membered heterocyclyl or 5- to 10-membered heteroaryl, wherein the heterocycle or heteroaryl contains 1 to 4 heteroatoms selected from O, S or N;
each F is independently selected from C₃₋₂₀ carbocyclyl, C₆₋₂₀ aryl, 3- to 20-membered heterocyclyl or 5- to 20-membered heteroaryl, wherein the heterocyclyl or heteroaryl contains 1 to 4 heteroatoms selected from O, S or N;
each R^{k2} is independently selected from a bond, —CO—, —SO₂—, —SO— or —C(R^{k3})₂—;
each R^{k1} is independently selected from H, F, Cl, Br, I, OH, ═O, NH₂, CN, COOH, CONH₂, C₁₋₆ alkyl or C₁₋₆ alkoxy, wherein the alkyl or alkoxy is optionally further substituted with 0 to 4 substituents selected from H, F, Cl, Br, I, OH, ═O, NH₂, CN, COOH, CONH₂, C₁₋₄ alkyl or C₁₋₄ alkoxy;
each R^{k3} is independently selected from H, F, Cl, Br, I, OH, ═O, NH₂, CN, COOH, CONH₂, C₁₋₆ alkyl, C₁₋₆ alkoxy, C₃₋₈ cycloalkyl or 3- to 8-membered heterocyclyl, wherein the alkyl, alkoxy, cycloalkyl or heterocyclyl is optionally further substituted with 0 to 4 substituents selected from H, F, Cl, Br, I, OH, ═O, NH₂, CN, COOH, CONH₂, C₁₋₄ alkyl or C₁₋₄ alkoxy, and the heterocyclyl contains 1 to 4 heteroatoms selected from O, S or N;
or two R^{k3} together with the carbon atoms or ring backbones to which they are directly attached form 3- to 8-membered carbocycle or 3- to 8-membered heterocycle, wherein the carbocycle or heterocycle is optionally further substituted with 0 to 4 substituents selected from H, F, Cl, Br, I, OH, ═O, NH₂, CN, COOH, CONH₂, C₁₋₄ alkyl or C₁₋₄ alkoxy, and the heterocycle contains 1 to 4 heteroatoms selected from O, S or N;
each R^{k4} is independently selected from H, OH, NH₂, CN, CONH₂, C₁₋₆ alkyl, C₃₋₈ cycloalkyl or 3- to 8-membered heterocyclyl, wherein the alkyl, cycloalkyl or heterocyclyl is optionally further substituted with 0 to 4 substituents selected from H, F, Cl, Br, I, OH, ═O, NH₂, CN, COOH, CONH₂, C₁₋₄ alkyl or C₁₋₄ alkoxy, and the heterocyclyl contains 1 to 4 heteroatoms selected from O, S or N;
M₁ is selected from a bond, —C(═O)NH—, —NHC(═O)—, —CH₂—C(═O)NH—, —C(═O)CH₂NH—, or 5- to 6-membered heteroaryl, wherein the heteroaryl is optionally further substituted with 0 to 4 substituents selected from H, F, Cl, Br, I, OH, ═O, CF₃, NH₂, CN, C₁₋₄ alkyl, halogen-substituted C₁₋₄ alkyl, hydroxyl-substituted C₁₋₄ alkyl or C₁₋₄ alkoxy, and the heteroaryl contains 1 to 4 heteroatoms selected from O, S or N;
M₂ is selected from —NHC(═O)—C₁₋₆ alkyl, —NHC(═O)—C₃₋₆ cycloalkyl or 4- to 10-membered heterocyclyl, wherein the alkyl, cycloalkyl or heterocyclyl is optionally further substituted with 0 to 4 substituents selected from H, F, Cl, Br, I, ═O, OH, NH₂, C₁₋₄ alkyl or C₁₋₄ alkoxy, and the heterocyclyl contains 1 to 4 heteroatoms selected from O, S or N;
M₃ is selected from —NH— or —O—;
R^{k10} is selected from C₁₋₆ alkyl, wherein the alkyl is optionally further substituted with 0 to 4 substituents selected from H, F, Cl, Br, I, ═O, OH, C₁₋₆ alkyl or C₃₋₆ cycloalkyl;
each R^{k11} is independently selected from H, F, Cl, Br, I, ═O, OH, SH, C₁₋₆ alkyl, C₁₋₆ alkoxy or C₁₋₆ alkylthio or —O—C(═O)—C₁₋₆ alkyl, wherein the alkyl, alkoxy or alkylthio is optionally further substituted with 0 to 4 substituents selected from H, F, Cl, Br, I, OH, C₁₋₄ alkyl or C₁₋₄ alkoxy;
R^{k12} and R^{k13} are each independently selected from H, C₁₋₆ alkyl or C₃₋₆ cycloalkyl, wherein the alkyl or cycloalkyl is optionally further substituted with 0 to 4 substituents selected from H, F, Cl, Br, I, ═O, OH, NH₂, C₁₋₄ alkyl or C₁₋₄ alkoxy;
R^{k14} is selected from 5- to 6-membered heteroaryl, wherein the heteroaryl is optionally further substituted with 0 to 4 substituents selected from H, F, Cl, Br, I, OH, ═O, CF₃, CN, C₁₋₄ alkyl, halogen-substituted C₁₋₄ alkyl, hydroxyl-substituted C₁₋₄ alkyl, C₁₋₄ alkoxy or C₃₋₆ cycloalkyl, and the heteroaryl contains 1 to 4 heteroatoms selected from N, O or S;
G is selected from 6- to 10-membered aryl or 5- to 10-membered heteroaryl, wherein the aryl or heteroaryl is optionally further substituted with 0 to 4 substituents selected from H, F, Cl, Br, I, OH, ═O, CF₃, CN, C₁₋₄ alkyl, halogen-substituted C₁₋₄ alkyl, hydroxyl-substituted C₁₋₄ alkyl, C₁₋₄ alkoxy or C₃₋₆ cycloalkyl, and the heteroaryl contains 1 to 4 heteroatoms selected from N, O or S;
optionally, 1 to 20 H of the compound of general formula (I) are replaced by 1 to 20 deuterium;
n1, n2 and n3 are each independently selected from 0, 1, 2 or 3;
each p1 or p2 is independently selected from 0, 1, 2, 3, 4 or 5.

As a second embodiment of the present invention, provided is the above-mentioned compound of general formula (I) or the stereoisomer, deuterated compound, solvate, prodrug, metabolite, pharmaceutically acceptable salt or co-crystal thereof, wherein
L is selected from -Cy1-Ak1-Cy2-Ak2-Cy3-Ak3-Cy4-Ak4-Cy5-Ak5-, -Cy1-Cy2-Cy3-Cy4-Ak1-Ak2-Ak3-Ak4-Ak5-, -Cy1-Ak1-Cy2-Ak2-Cy3-Ak3-Cy4-Ak4-Ak5-, -Ak1-Cy1-Ak2-Cy2-Ak3-Cy3-Ak4-Cy4-Ak5-, -Cy1-Ak1-Cy2-Ak2-Cy3-Cy4-Ak3-Ak4-Ak5-, -Cy1-Ak1-Cy2-Ak2-Ak3-Cy3-Cy4-Ak4-Ak5-, -Cy1-Ak1-Ak2-Ak3-Ak4-Ak5-Cy2-Cy3-Cy4-, -Cy1-Cy2-Ak1-Ak2-Ak3-Ak4-Ak5-Cy3-Cy4-, -Cy1-Cy2-Cy3-Ak1-Ak2-Ak3-Ak4-Ak5-Cy4-, -Cy1-Cy2-Cy3-Cy4-Ak1-Ak2-Ak3-Ak4-Ak5-, -Cy1-Ak1-Cy2-Cy3-Cy4-Ak2-Ak3-Ak4-Ak5-, -Cy1-Cy2-Ak1-Cy3-Cy4-Ak2-Ak3-Ak4-Ak5-, -Cy1-Cy2-Cy3-Ak1-Cy4-Ak2-Ak3-Ak4-Ak5-, -Cy1-Ak1-Ak2-Cy2-Cy3-Cy4-Ak3-Ak4-Ak5-, -Cy1-Cy2-Ak1-Ak2-Cy3-Cy4-Ak3-Ak4-Ak5-, -Cy1-Cy2-Cy3-Ak1-Ak2-Cy4-Ak3-Ak4-Ak5-, -Cy1-Ak1-Ak2-Ak3-Cy2-Cy3-Cy4-Ak4-Ak5-, -Cy1-Cy2-Ak1-Ak2-Ak3-Cy3-Cy4-Ak4-Ak5-, -Cy1-Cy2-Cy3-Ak1-Ak2-Ak3-Cy4-Ak4-Ak5-, -Cy1-Ak1-Ak2-Ak3-Ak4-Cy2-Cy3-Cy4-Ak5-, -Cy1-Cy2-Ak1-Ak2-Ak3-Ak4-Cy3-Cy4-Ak5-, -Cy1-Cy2-Cy3-Ak1-Ak2-Ak3-Ak4-Cy4-Ak5-, -Ak1-Ak2-Ak3-Ak4-Ak5-Cy1-Cy2-Cy3-Cy4-, -Ak1-Cy1-Cy2-Cy3-Cy4-Ak2-Ak3-Ak4-Ak5-, -Ak1-Ak2-Cy1-Cy2-Cy3-Cy4-Ak3-Ak4-Ak5-, -Ak1-Ak2-Ak3-Cy1-Cy2-Cy3-Cy4-Ak4-Ak5-, -Ak1-Ak2-Ak3-Ak4-Cy1-Cy2-Cy3-Cy4-Ak5-, -Ak1-Cy1-Ak2-Ak3-Ak4-Ak5-Cy2-Cy3-Cy4-, -Ak1-Cy1-Cy2-Ak2-Ak3-Ak4-Ak5-Cy3-Cy4-, -Ak1-Cy1-Cy2-Cy3-Ak2-Ak3-Ak4-Ak5-Cy4-, -Ak1-Ak2-Cy1-Ak3-Ak4-Ak5-Cy2-Cy3-Cy4-, -Ak1-Ak2-Cy1-Cy2-Ak3-Ak4-Ak5-Cy3-Cy4-, -Ak1-Ak2-Cy1-Cy2-Cy3-Ak3-Ak4-Ak5-Cy4-, -Ak1-Ak2-Ak3-Cy1-Ak4-Ak5-Cy2-Cy3-Cy4-, -Ak1-Ak2-Ak3-Cy1-Cy2-Ak4-Ak5-Cy3-Cy4-, -Ak1-Ak2-Ak3-Cy1-Cy2-Cy3-Ak4-Ak5-Cy4-, -Ak1-Ak2-Ak3-Ak4-Cy1-Ak5-Cy2-Cy3-Cy4-, -Ak1-Ak2-Ak3-Ak4-Cy1-Cy2-Ak5-Cy3-Cy4-, -Ak1-Ak2-Ak3-Ak4-Cy1-Cy2-Cy3-Ak5-Cy4-, -Ak1-, -Ak1-Ak2-, -Ak1-Ak2-Ak3-, -Ak1-Ak2-Ak3-Ak4-, -Ak1-Ak2-Ak3-Ak4-Ak5-, -Ak1-Ak2-Ak3-Ak4-Ak5-Ak6-, -Ak1-Ak2-Ak3-Ak4-Ak5-Ak6-Ak7-, -Ak1-Ak2-Ak3-Ak4-Ak5-Ak6-Ak7-Ak8-, or -Ak1-Ak2-Ak3-Ak4-Ak5-Ak6-Ak7-Ak8-Ak9;
Ak1, Ak2, Ak3, Ak4, Ak5, Ak6, Ak7, Ak8 and Ak9 are each independently selected from —(CH₂)_{q}—, —(CH₂)_{q}—O—, —O—(CH₂)_{q}—, —(CH₂)_{q}—NR^{L}—, —NR^{L}—(CH₂)_{q}—, —(CH₂)_{q}—NR^{L}C(═O)—, —(CH₂)_{q}—C(═O)NR^{L}—, —C(═O)—, —C(═O)—(CH₂)_{q}—NR^{L}—, —(C≡C)_{q}— or a bond, wherein the —CH₂— is optionally further substituted with 0 to 2 substituents selected from H, halogen, OH, CN, NH₂, C₁₋₄ alkyl, C₁₋₄ alkoxy, halogen-substituted C₁₋₄ alkyl, hydroxyl-substituted C₁₋₄ alkyl or cyano-substituted C₁₋₄ alkyl;
each Cy1, Cy2, Cy3, Cy4 or Cy5 is independently selected from a bond, a 4- to 7-membered mono-heterocyclic ring, a 4- to 10-membered fused-heterocyclic ring, a 5- to 12-membered spiro-heterocyclic ring, a 7- to 10-membered bridged-heterocyclic ring, 3- to 7-membered monocycloalkyl, 4- to 10-membered fused cycloalkyl, 5- to 12-membered spiro cycloalkyl, 7- to 10-membered bridged cycloalkyl, 5- to 10-membered heteroaryl or 6- to 10-membered aryl, wherein the aryl, heteroaryl, cycloalkyl, mono-heterocyclic ring, fused-heterocyclic ring, spiro-heterocyclic ring or bridged-heterocyclic ring is optionally further substituted with 0 to 4 substituents selected from H, F, Cl, Br, I, OH, COOH, CN, NH₂, ═O, C₁₋₄ alkyl, halogen-substituted C₁₋₄ alkyl, hydroxyl-substituted C₁₋₄ alkyl or C₁₋₄ alkoxy, and the heteroaryl, mono-heterocyclic ring, fused-heterocyclic ring, spiro-heterocyclic ring or bridged-heterocyclic ring contains 1 to 4 heteroatoms selected from O, S or N, and is optionally further substituted with 0, 1 or 2 ═O when the heteroatom is selected from S;
each q is independently selected from 0, 1, 2, 3 or 4;
each R^{L} is independently selected from H or C₁₋₆ alkyl;
the definitions of other groups are the same as those in the first embodiment of the present invention.

As a third embodiment of the present invention, provided is the above-mentioned compound of general formula (I) or the stereoisomer, deuterated compound, solvate, prodrug, metabolite, pharmaceutically acceptable salt or co-crystal thereof, wherein Ak1, Ak2, Ak3, Ak4, Ak5, Ak6, Ak7, Ak8 and Ak9 are each independently selected from —(CH₂)_{q}—, —(CH₂)_{q}—O—, —O—(CH₂)_{q}—, —(CH₂)_{q}—NR^{L}—, —NR^{L}—(CH₂)_{q}—, —(CH₂)_{q}—NR^{L}C(═O)—, —(CH₂)_{q}—C(═O)NR^{L}—, —C(═O)—, —C(═O)—(CH₂)_{q}—NR^{L}—, —(C≡C)_{q}—, or a bond, wherein the —CH₂— is optionally further substituted with 0 to 2 substituents selected from H, F, Cl, Br, I, OH, CN, NH₂, CF₃, hydroxymethyl, C₁₋₄ alkyl, or C₁₋₄ alkoxy; each Cy1, Cy2, Cy3, Cy4 or Cy5 is independently selected from a bond, a 4- to 7-membered nitrogen-containing mono-heterocyclic ring, a 4- to 10-membered nitrogen-containing fused-heterocyclic ring, a 5- to 12-membered nitrogen-containing spiro-heterocyclic ring, a 7- to 10-membered nitrogen-containing bridged-heterocyclic ring, 3- to 7-membered monocycloalkyl, 4- to 10-membered fused cycloalkyl, 5- to 12-membered spiro cycloalkyl, 7- to 10-membered bridged cycloalkyl, 5- to 10-membered heteroaryl or 6- to 10-membered aryl, wherein the mono-heterocyclic ring, fused-heterocyclic ring, bridged-heterocyclic ring, spiro-heterocyclic ring, cycloalkyl, aryl or heteroaryl is optionally further substituted with 0 to 4 substituents selected from H, F, Cl, Br, I, OH, COOH, CN, NH₂, ═O, C₁₋₄ alkyl, halogen-substituted C₁₋₄ alkyl, hydroxyl-substituted C₁₋₄ alkyl or C₁₋₄ alkoxy, and the mono-heterocyclic ring, fused-heterocyclic ring, bridged-heterocyclic ring, spiro-heterocyclic ring or heteroaryl contains 1 to 4 heteroatoms selected from O, S or N, and is optionally further substituted with 0, 1 or 2 ═O when the heteroatom is selected from S; each R^{L} is independently selected from H or C₁₋₄ alkyl;
K is selected from

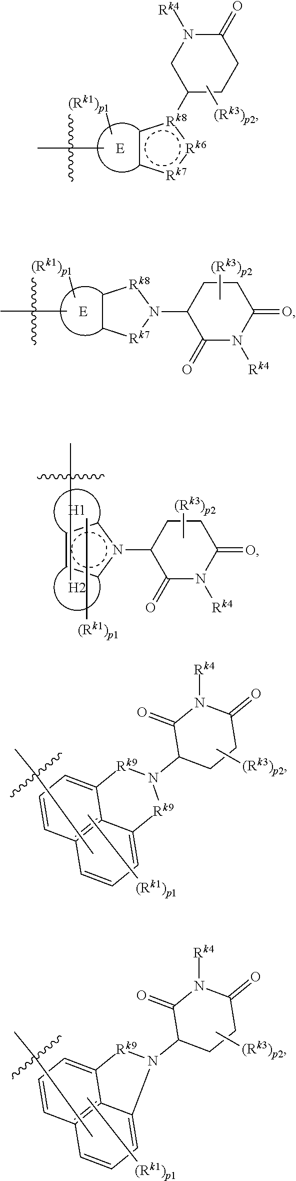

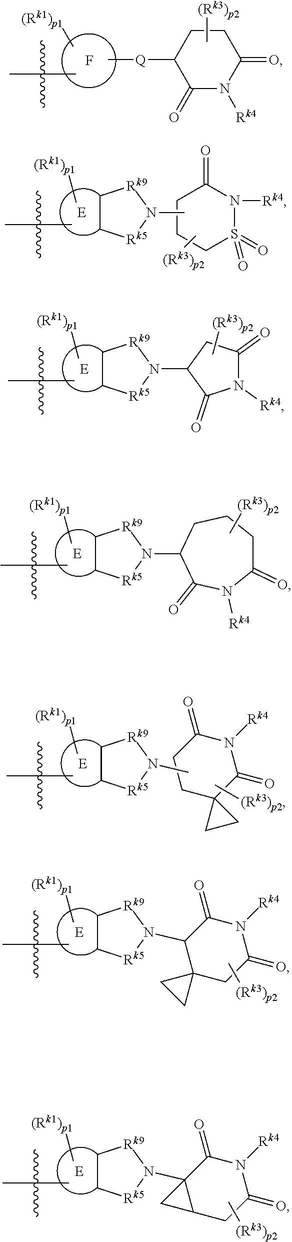

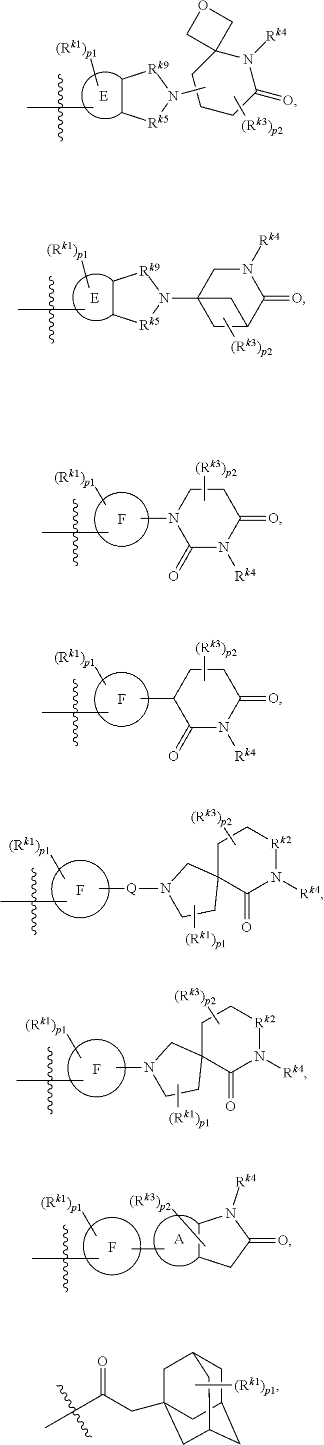

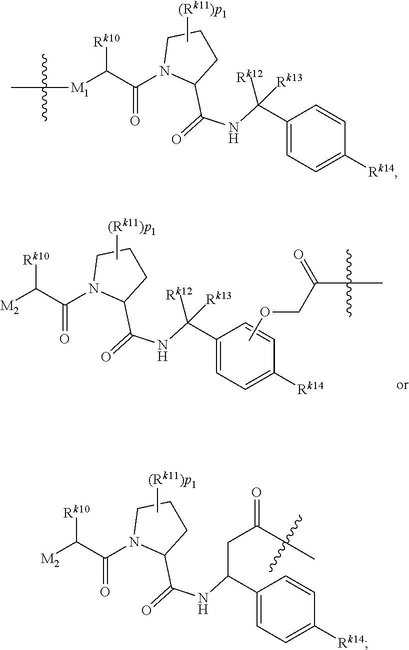

[symbol] represents a ring selected from an aromatic ring or a non-aromatic ring;
M₂ is selected from —NHC(═O)—C₁₋₄ alkyl, —NHC(═O)—C₃₋₆ cycloalkyl or 4- to 10-membered heterocyclyl, wherein the alkyl, cycloalkyl or heterocyclyl is optionally further substituted with 0 to 4 substituents selected from H, F, Cl, Br, I, ═O, OH, NH₂, C₁₋₄ alkyl or C₁₋₄ alkoxy, and the heterocyclyl contains 1 to 4 heteroatoms selected from O, S or N;
R^{k10} is selected from C₁₋₄ alkyl, wherein the alkyl is optionally further substituted with 0 to 4 substituents selected from H, F, Cl, Br, I, ═O, OH, C₁₋₄ alkyl or C₃₋₆ cycloalkyl;
each R^{k11} is independently selected from H, F, Cl, Br, I, ═O, OH, SH, C₁₋₄ alkyl, C₁₋₄ alkoxy or C₁₋₄ alkylthio or —O—C(═O)—C₁₋₄ alkyl, wherein the alkyl, alkoxy or alkylthio is optionally further substituted with 0 to 4 substituents selected from H, F, Cl, Br, I, OH, C₁₋₄ alkyl or C₁₋₄ alkoxy;
R^{k12} and R^{k13} are each independently selected from H, C₁₋₄ alkyl or C₃₋₆ cycloalkyl, wherein the alkyl or cycloalkyl is optionally further substituted with 0 to 4 substituents selected from H, F, Cl, Br, I, ═O, OH, NH₂, C₁₋₄ alkyl or C₁₋₄ alkoxy;
each Q is independently selected from —O—, —S—, —CH₂—, —NR^{q}—, —CO—, —NR^{q}CO—, —CONR^{q}— or 4- to 7-membered heterocyclyl, wherein the heterocyclyl is optionally further substituted with 0 to 4 substituents selected from H, F, Cl, Br, I, OH, ═O, NH₂, CN, COOH, CONH₂, C₁₋₄ alkyl or C₁₋₄ alkoxy, and the heterocyclyl contains 1 to 4 heteroatoms selected from O, S or N;
R^{q} is selected from H or C₁₋₄ alkyl;
R^{k1} and R^{k3} are each independently selected from H, F, Cl, Br, I, OH, ═O, NH₂, CF₃, CN, COOH, CONH₂, C₁₋₄ alkyl or C₁₋₄ alkoxy, wherein the alkyl or alkoxy is optionally further substituted with 0 to 4 substituents selected from H, F, Cl, Br, I, OH, or NH₂;
or two R^{k3} together with the carbon atoms or ring backbones to which they are directly attached form 3- to 6-membered carbocycle or 3- to 7-membered heterocycle, wherein the carbocycle or heterocycle is optionally further substituted with 0 to 4 substituents selected from H, F, Cl, Br, I, OH, ═O, NH₂, CN, COOH, CONH₂, C₁₋₄ alkyl or C₁₋₄ alkoxy, and the heterocycle contains 1 to 4 heteroatoms selected from O, S or N;
each R^{k4} is independently selected from H, OH, NH₂, CF₃, CN or C₁₋₄ alkyl;
each R^{k5} is independently selected from CO, CH₂, SO₂ or

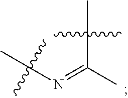 [SMILES: */N=C(\*)C]

each R^{k6} is independently selected from CO, CH, SO, SO₂, CH₂ or N;
each R^{k7} is independently selected from CO, CH, N, CH₂, O, S, N(CH₃) or NH;
each R^{V8} is independently selected from C, N or CH;
each R^{k}s is independently selected from CO, CH₂ or SO₂;
each A, H1 or H2 is independently selected from C₃₋₈ carbocycle, a benzene ring, 4- to 7-membered heterocycle or 5- to 6-membered heteroaryl, wherein the heterocycle or heteroaryl contains 1 to 4 heteroatoms selected from O, S or N;
each E is independently selected from C₃₋₈ carbocycle, a benzene ring, 4- to 7-membered heterocycle, 8- to 12-membered heterocycle, 7- to 12-membered heteroaryl or 5- to 6-membered heteroaryl, wherein the heterocycle or heteroaryl contains 1 to 4 heteroatoms selected from O, S or N;
each F is independently selected from 3- to 7-membered monocycloalkyl, 4- to 10-membered fused cycloalkyl, 5- to 12-membered spiro cycloalkyl, 5- to 10-membered bridged cycloalkyl, a 4- to 7-membered mono-heterocyclic ring, a 4- to 10-membered fused-heterocyclic ring, a 5- to 12-membered spiro-heterocyclic ring, a 5- to 10-membered bridged-heterocyclic ring, C₆₋₁₄ aryl or 5- to 10-membered heteroaryl, wherein the mono-heterocyclic ring, fused-heterocyclic ring, spiro-heterocyclic ring, bridged-heterocyclic ring or heteroaryl contains 1 to 4 heteroatoms selected from O, S or N;
the definitions of other groups are the same as those in either the first or second embodiment of the present invention.

As a fourth embodiment of the present invention, provided is the above-mentioned compound of general formula (I) or the stereoisomer, deuterated compound, solvate, prodrug, metabolite, pharmaceutically acceptable salt or co-crystal thereof, wherein
Ak1, Ak2, Ak3, Ak4, Ak5, Ak6, Ak7, Ak8 and Ak9 are each independently selected from —(CH₂)_{q}—, —(CH₂)_{q}—O—, —O—(CH₂)_{q}—, —(CH₂)_{q}—NR^{L}—, —NR^{L}—(CH₂)_{q}—, —(CH₂)_{q}—NR^{L}C(═O)—, —(CH₂)_{q}—C(═O)NR^{L}—, —C(═O)—, —C(═O)—(CH₂)_{q}—NR^{L}—, —(C≡C)_{q}—, or a bond, wherein the —CH₂— is optionally further substituted with 0 to 2 substituents selected from H, F, Cl, Br, I, OH, CN, NH₂, CF₃, hydroxymethyl, methyl, ethyl, methoxy or ethoxy;
R^{L} is selected from H, methyl or ethyl;
each q is independently selected from 0, 1, 2 or 3;
each Cy1, Cy2, Cy3, Cy4 or Cy5 is independently selected from a bond or one of the following substituted or unsubstituted groups: cyclopropyl, cyclobutyl, cyclopentyl, cyclohexyl, azetidinyl, azacyclopentyl, azacyclohexenyl, piperidyl, morpholinyl, piperazine, phenyl, cyclopropyl-fused-cyclopropyl, cyclopropyl-fused-cyclobutyl, cyclopropyl-fused-cyclopentyl, cyclopropyl-fused-cyclohexyl, cyclobutyl-fused-cyclobutyl, cyclobutyl-fused-cyclopentyl, cyclobutyl-fused-cyclohexyl, cyclopentyl-fused-cyclopentyl, cyclopentyl-fused-cyclohexyl, cyclohexyl-fused-cyclohexyl, cyclopropyl-spiro-cyclopropyl, cyclopropyl-spiro-cyclobutyl, cyclopropyl-spiro-cyclopentyl, cyclopropyl-spiro-cyclohexyl, cyclobutyl-spiro-cyclobutyl, cyclobutyl-spiro-cyclopentyl, cyclobutyl-spiro-cyclohexyl, cyclopentyl-spiro-cyclopentyl, cyclopentyl-spiro-cyclohexyl, cyclohexyl-spiro-cyclohexyl, cyclopropyl-fused-azetidinyl, cyclopropyl-fused-azacyclopentyl, cyclopropyl-fused-azacyclohexyl, cyclopropyl-fused-piperidyl, cyclobutyl-fused-azetidinyl, cyclobutyl-fused-azacyclopentyl, cyclobutyl-fused-azacyclohexyl, cyclobutyl-fused-piperidyl, cyclopentyl-fused-azetidinyl, cyclopentyl-fused-azacyclopentyl, cyclopentyl-fused-azacyclohexyl, cyclopentyl-fused-piperidyl, cyclohexyl-fused-azetidinyl, cyclohexyl-fused-azacyclopentyl, cyclohexyl-fused-azacyclohexyl, cyclohexyl-fused-piperidyl, azetidinyl-fused-azetidinyl, azetidinyl-fused-azacyclopentyl, azetidinyl-fused-azacyclohexyl, azetidinyl-fused-piperidyl, azacyclopentyl-fused-azetidinyl, azacyclopentyl-fused-azacyclopentyl, azacyclopentyl-fused-azacyclohexyl, azacyclopentyl-fused-piperidyl, azacyclohexyl-fused-azetidinyl, azacyclohexyl-fused-azacyclopentyl, azacyclohexyl-fused-azacyclohexyl, azacyclohexyl-fused-piperidyl, cyclobutyl-spiro-azetidinyl, cyclobutyl-spiro-azacyclopentyl, cyclobutyl-spiro-azacyclohexyl, cyclopentyl-spiro-azetidinyl, cyclopentyl-spiro-azacyclopentyl, cyclopentyl-spiro-azacyclohexyl, cyclohexyl-spiro-azetidinyl, cyclohexyl-spiro-azacyclopentyl, cyclohexyl-spiro-azacyclohexyl, azetidinyl-spiro-azetidinyl, azetidinyl-spiro-azacyclopentyl, azetidinyl-spiro-azacyclohexyl, azacyclopentyl-spiro-azetidinyl, azacyclopentyl-spiro-azacyclopentyl, azacyclopentyl-spiro-azacyclohexyl, azacyclohexyl-spiro-azetidinyl, azacyclohexyl-spiro-azacyclopentyl, azacyclohexyl-spiro-azacyclohexyl, cyclobutyl-spiro-piperidyl, cyclopentyl-spiro-piperidyl, cyclohexyl-spiro-piperidyl, azetidinyl-spiro-piperidyl, azacyclopentyl-spiro-piperidyl, azacyclohexyl-spiro-piperidyl,

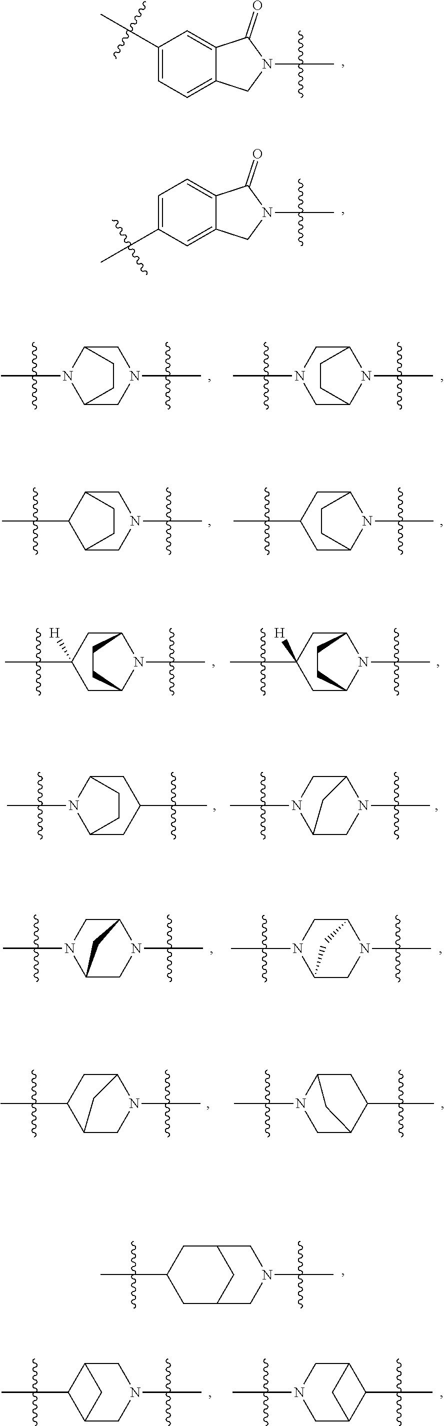

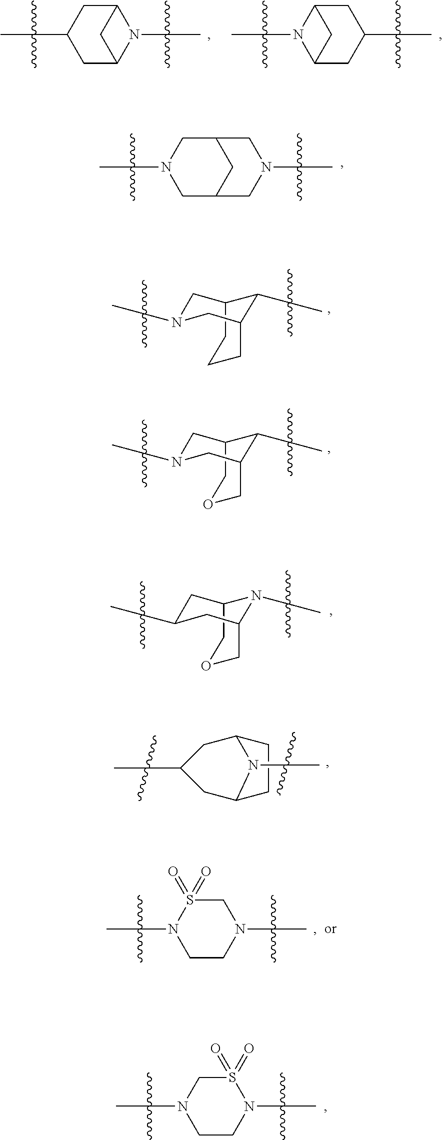

which, when substituted, is optionally further substituted with 0 to 4 substituents selected from H, F, Cl, Br, I, OH, NH₂, COOH, CN, ═O, C₁₋₄ alkyl, halogen-substituted C₁₋₄ alkyl, hydroxyl-substituted C₁₋₄ alkyl, or C₁₋₄ alkoxy;
D is selected from ethylene;
B₁ and Z are each independently selected from azetidinyl, azacyclopentyl, piperazinyl, piperidyl, azacyclohexenyl, azepanyl, 1,4-diazepanyl, cyclobutyl-fused-azetidinyl, cyclobutyl-fused-azacyclopentyl, cyclobutyl-fused-azacyclohexyl, cyclobutyl-fused-piperidyl, cyclopentyl-fused-azetidinyl, cyclopentyl-fused-azacyclopentyl, cyclopentyl-fused-azacyclohexyl, cyclopentyl-fused-piperidyl, cyclohexyl-fused-azetidinyl, cyclohexyl-fused-azacyclopentyl, cyclohexyl-fused-azacyclohexyl, cyclohexyl-fused-piperidyl, azetidinyl-fused-azetidinyl, azacyclopentyl-fused-azetidinyl, azacyclopentyl-fused-azacyclopentyl, azacyclopentyl-fused-azacyclohexyl, azacyclopentyl-fused-piperidyl, azacyclohexyl-fused-azetidinyl, azacyclohexyl-fused-azacyclopentyl, azacyclohexyl-fused-azacyclohexyl, azacyclohexyl-fused-piperidyl, cyclobutyl-spiro-azetidinyl, cyclobutyl-spiro-azacyclopentyl, cyclobutyl-spiro-azacyclohexyl, cyclopentyl-spiro-azetidinyl, cyclopentyl-spiro-azacyclopentyl, cyclopentyl-spiro-azacyclohexyl, cyclohexyl-spiro-azetidinyl, cyclohexyl-spiro-azacyclopentyl, azetidinyl-spiro-azetidinyl, azetidinyl-spiro-azacyclopentyl, azetidinyl-spiro-azacyclohexyl, azacyclopentyl-spiro-azetidinyl, azacyclopentyl-spiro-azacyclopentyl, azacyclopentyl-spiro-azacyclohexyl, azacyclohexyl-spiro-azetidinyl, azacyclohexyl-spiro-azacyclopentyl, azacyclohexyl-spiro-azacyclohexyl, cyclobutyl-spiro-piperidyl, cyclopentyl-spiro-piperidyl, cyclohexyl-spiro-piperidyl, azetidinyl-spiro-piperidyl, azacyclopentyl-spiro-piperidyl, azacyclohexyl-spiro-piperidyl,

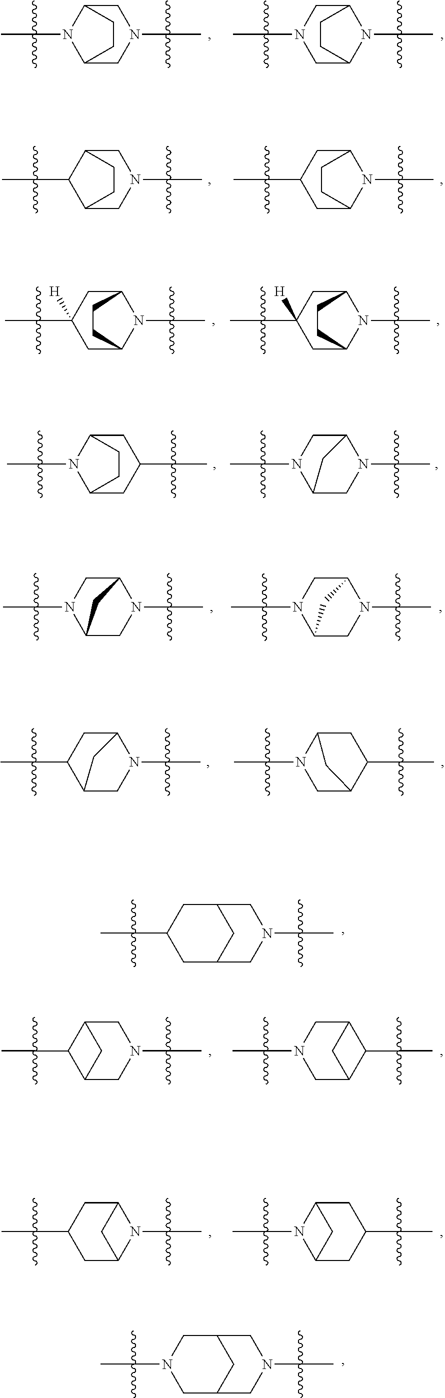

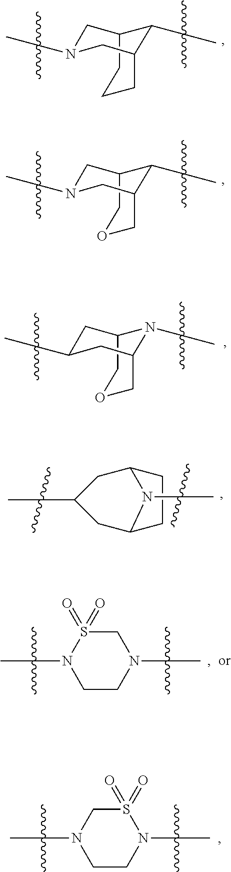 [SMILES: *C1C2CCCC1CN(*)C2 ; *C1C2COCC1CN(*)C2 ; *C1CC2COCC(C1)N2* ; *C1CC2CCC(C1)N2* ; *N1CCN(*)S(=O)(=O)C1 ; *N1CCN(*)S(=O)(=O)C1]

the B₁ is optionally further substituted with 0 to 4 R^{B1}, and the Z is optionally further substituted with 0 to 4 R^{Q};
B₂ and B₄ are each independently selected from phenyl or 5- to 6-membered heteroaryl, B₃ and B₅ are each independently selected from phenyl, naphthyl, 5- to 6-membered heteroaryl or benzo 5- to 6-membered heteroaryl, the B₂ is optionally further substituted with 0 to 4 R^{B2}, the B₃ is optionally further substituted with 0 to 5 R^{B3}, the B₄ is optionally further substituted with 0 to 4 R^{B4}, and the B₅ is optionally further substituted with 0 to 5 R^{B5}, wherein the heteroaryl contains 1 to 3 heteroatoms selected from O, S or N;
R^{B1}, R^{Q}, R^{B2}, R^{B3} and R^{B5} are each independently selected from F, Cl, Br, I, oxo, OH, CN, methyl, ethyl, methoxy or ethoxy, wherein the methyl, ethyl, methoxy or ethoxy is optionally further substituted with 0 to 4 substituents selected from H, halogen, OH, CN or C₁₋₄ alkyl;
each R^{B4} is independently selected from —SO₂-methyl, —SO₂-ethyl, nitro, F, Cl, Br, I, OH, CN, methyl, ethyl, methoxy or ethoxy, wherein the methyl, ethyl, methoxy or ethoxy is optionally further substituted with 0 to 4 substituents selected from H, halogen, OH, CN or C₁₋₄ alkyl;
R^{w1}, R^{w2} and R^{w5} are each independently selected from H, F, Cl, Br, I, OH, CN, methyl, ethyl, methoxy or ethoxy, wherein the methyl, ethyl, methoxy or ethoxy is optionally further substituted with 0 to 4 substituents selected from H, halogen, OH, CN or C₁₋₄ alkyl;
alternatively, R^{w1} and R^{w2} are directly connected to form C₃₋₆ carbocycle or 3- to 6-membered heterocycle, wherein the carbocycle or heterocycle is optionally further substituted with 0 to 4 substituents selected from H, halogen, OH, CN or C₁₋₄ alkyl, and the heterocycle contains 1 to 3 heteroatoms selected from O, S or N;
R^{w3} and R^{w4} are directly connected to form C₃₋₆ carbocycle or 3- to 6-membered heterocycle, wherein the carbocycle or heterocycle is optionally further substituted with 0 to 4 substituents selected from H, halogen, OH, CN or C₁₋₄ alkyl, and the heterocycle contains 1 to 3 heteroatoms selected from O, S or N;
alternatively, R^{B1} and R^{B2} are directly connected to form C₅₋₇ carbocycle or 5- to 7-membered heterocycle, wherein the carbocycle or heterocycle is optionally further substituted with 0 to 3 R^{c}, and the heterocycle contains 1 to 3 heteroatoms selected from O, S or N;
each R^{c} is independently selected from F, Cl, Br, I, OH, CN, ═O, methyl, ethyl, methoxy, ethoxy, cyclopropyl, cyclobutyl, cyclopentyl, cyclohexyl, oxetanyl, oxacyclopentyl, oxacyclohexyl, azetidinyl, azacyclopentyl, azacyclohexyl, morpholinyl, or piperazinyl, wherein the methyl, ethyl, methoxy, ethoxy, cyclopropyl, cyclobutyl, cyclopentyl, cyclohexyl, oxetanyl, oxacyclopentyl, oxacyclohexyl, azetidinyl, azacyclopentyl, azacyclohexyl, morpholinyl, or piperazinyl is optionally further substituted with 0 to 4 substituents selected from H, halogen, OH, CN, C₁₋₄ alkyl, C₁₋₄ alkoxy, C₃₋₆ cycloalkyl, or 3- to 7-membered heterocycloalkyl, and the heterocycloalkyl contains 1 to 3 heteroatoms selected from O, S or N;
K is selected from

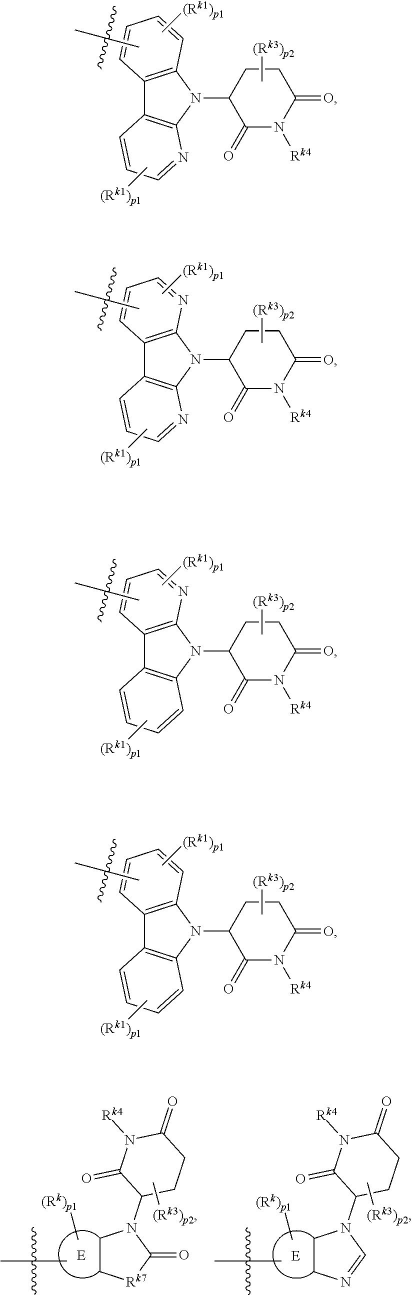

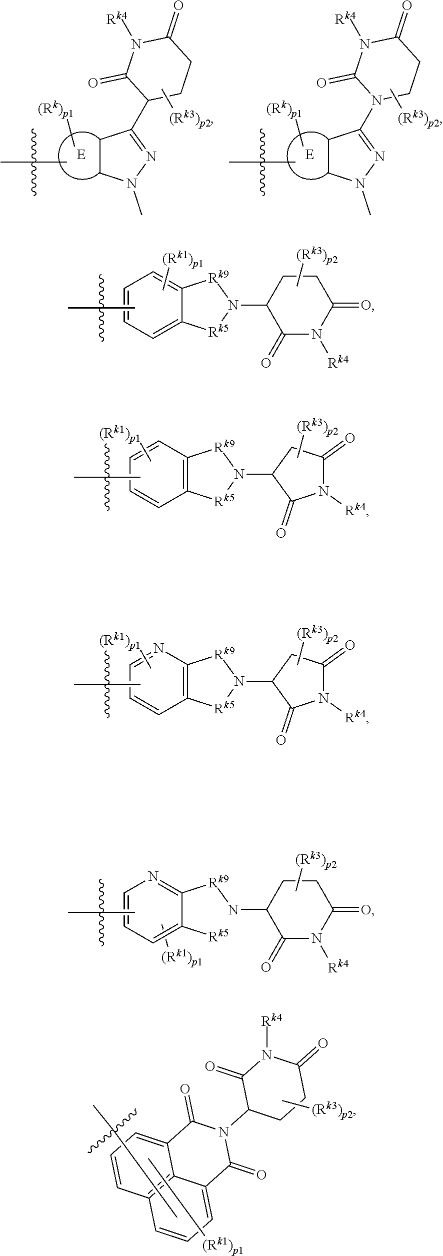

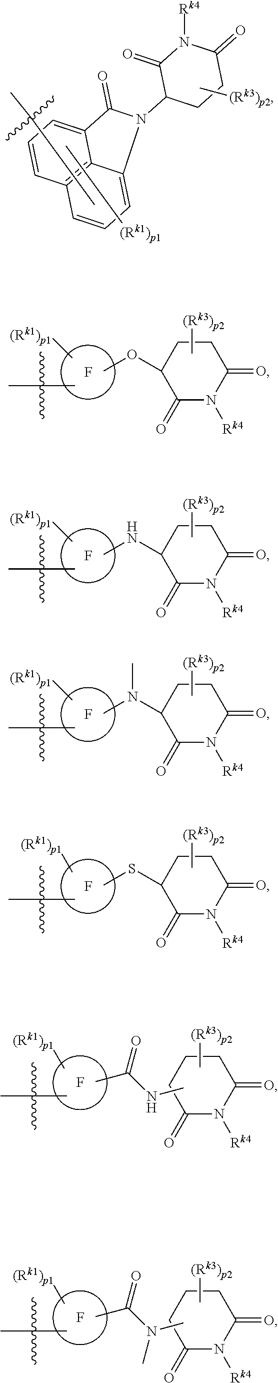

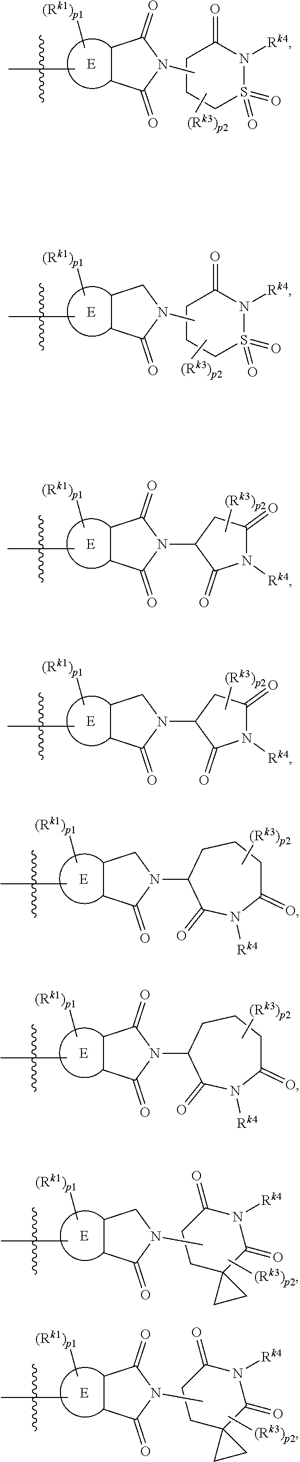

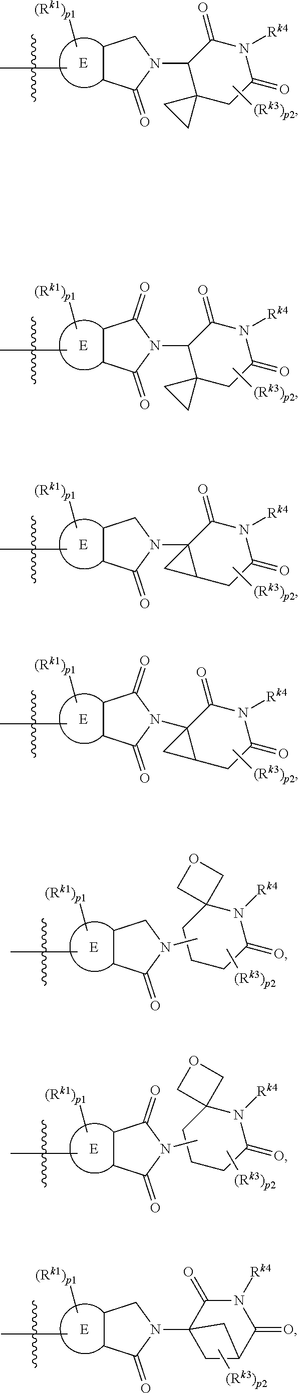

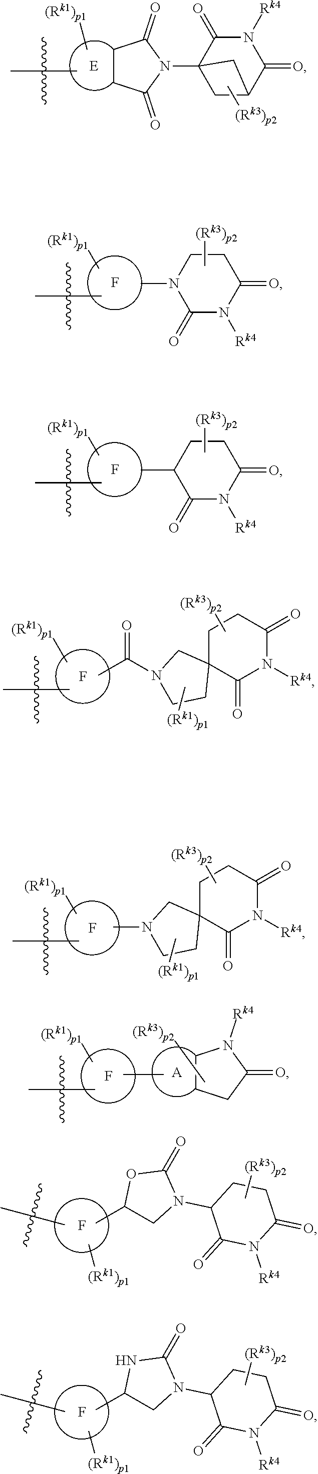

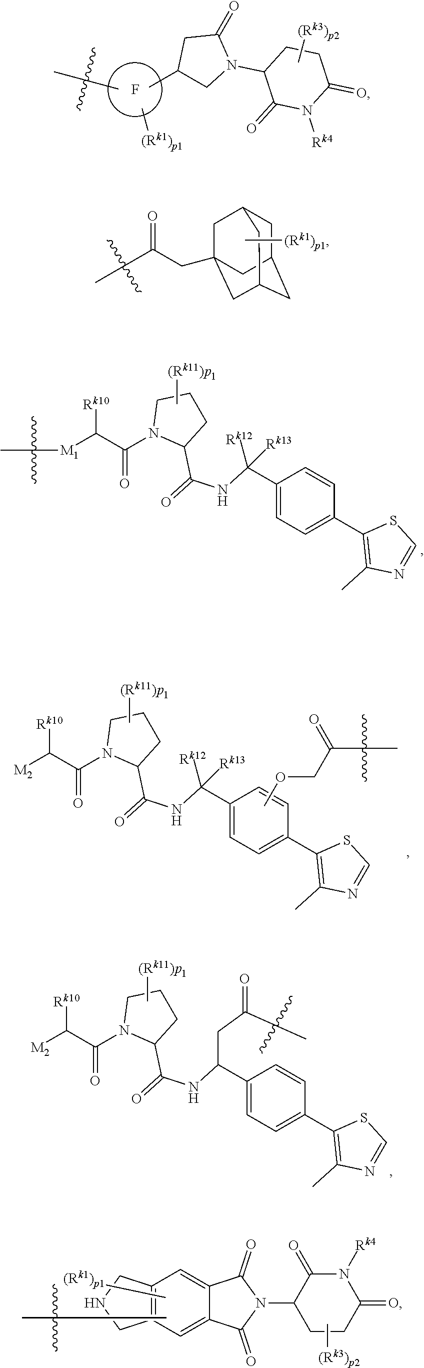

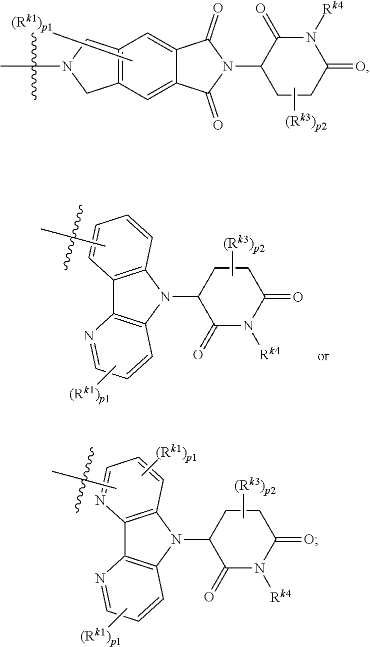

M₁ is selected from a bond, —C(═O)NH—, —CH₂—C(═O)NH—, —C(═O)CH₂NH—, pyrrolyl, pyrazolyl, imidazolyl, triazolyl, oxazolyl, isoxazolyl, furyl, thienyl, or thiazolyl;
M₂ is selected from —NHC(═O)—CH₃, —NHC(═O)-cyclopropyl, —NHC(═O)-cyclobutyl, azetidinyl, azacyclopentyl, benzo-azacyclopentyl or benzo-azacyclohexyl, wherein the cyclopropyl, cyclobutyl, azetidinyl, azacyclopentyl, benzo-azacyclopentyl or benzo-azacyclohexyl is optionally further substituted with 0 to 4 substituents selected from H, F, Cl, Br, I, ═O, OH, NH₂, C₁₋₄ alkyl or C₁₋₄ alkoxy;
R^{k10} is selected from methyl, ethyl, isopropyl, propyl or tert-butyl, wherein the methyl, ethyl, isopropyl, propyl or tert-butyl is optionally further substituted with 0 to 4 substituents selected from H, F, Cl, Br, I, ═O, OH, C₁₋₄ alkyl or C₃₋₆ cycloalkyl;
each R^{k11} is independently selected from H, F, Cl, Br, I, ═O, OH, SH, methyl, ethyl, isopropyl, propyl, methoxy, ethoxy, propoxy, isopropyloxy, methylthio, ethylthio, propylthio or —O—C(═O)—CH₃, wherein the methyl, ethyl, isopropyl, propyl, methoxy, ethoxy, propoxy, isopropyloxy, methylthio, ethylthio, or propylthio is optionally further substituted with 0 to 4 substituents selected from H, F, Cl, Br, I, OH, C₁₋₄ alkyl or C₁₋₄ alkoxy;
R^{k12} and R^{k13} are each independently selected from H, methyl, ethyl, isopropyl, propyl, cyclopropyl or cyclobutyl, wherein the methyl, ethyl, isopropyl, propyl, cyclopropyl or cyclobutyl is optionally further substituted with 0 to 4 substituents selected from H, F, Cl, Br, I, ═O, OH, NH₂, C₁₋₄ alkyl or C₁₋₄ alkoxy;
each E (ring E) is independently selected from phenyl, pyridyl, pyridazinyl, pyrazinyl, pyrimidyl, pyrrolyl, pyrazolyl, imidazolyl, thiazolyl, furyl, thienyl or oxazolyl;
each A is independently selected from phenyl, pyridyl, pyridazinyl, pyrazinyl, pyrimidyl, pyrrolyl, pyrazolyl, imidazolyl, thiazolyl, furyl, thienyl or oxazolyl;
each F is independently selected from cyclopropyl, cyclobutyl, cyclopentyl, cyclohexyl, bicyclo[1.1.1]pentanyl, 6,7-dihydro-5H-cyclopenta[c]pyridyl, 2,3-dihydro-1H-indenyl, phenyl, naphthyl, anthryl, phenanthryl, azetidinyl, azacyclopentyl, piperidyl, morpholinyl, pyridyl, pyrimidyl, pyridazinyl, pyrazinyl, triazinyl, pyrrolyl, pyrazolyl, imidazolyl, triazolyl, oxazolyl, furyl, thienyl, thiazolyl, benzoimidazolyl, benzopyrazolyl, benzothiazolyl, benzothienyl, benzofuryl, benzopyrrolyl, benzopyridyl, benzopyrazinyl, benzopyrimidyl, benzopyridazinyl, pyrrolopyrrolyl, pyrrolopyridyl, pyrrolopyrimidyl, pyrrolopyridazinyl, pyrrolopyrazinyl, imidazopyrimidyl, imidazopyridyl, imidazopyrazinyl, imidazopyridazinyl, pyrazolopyridyl, pyrazolopyrimidyl, pyrazolopyridazinyl, pyrazolopyrazinyl, pyrimidopyridyl, pyrimidopyrazinyl, pyrimidopyridazinyl, pyrimidopyrimidyl, pyridopyridyl, pyridopyrazinyl, pyridopyridazinyl, pyridazinopyridazinyl, pyridazinopyrazinyl or pyrazinopyrazinyl;
each R^{k7} is independently selected from CH₂, O, N(CH₃) or NH;
each p1 or p2 is independently selected from 0, 1 or 2;
the definitions of other groups are the same as those in any one of the first, second and third embodiments of the present invention.

As a fifth embodiment of the present invention, provided is the above-mentioned compound of general formula (I) or the stereoisomer, deuterated compound, solvate, prodrug, metabolite, pharmaceutically acceptable salt or co-crystal thereof, wherein
Ak1, Ak2, Ak3, Ak4, Ak5, Ak6, Ak7, Ak8 and Ak9 are each independently selected from a bond, —O—, —OCH₂—, —CH₂O—, —OCH₂CH₂—, —CH₂CH₂O—, —C≡C—, —C(CH₃)₂—, —CH₂—, —CH₂CH₂—, —CH₂CH₂CH₂—, —N(CH₃)—, —NH—, —CH₂N(CH₃)—, —CH₂NH—, —NHCH₂—, —CH₂CH₂N(CH₃)—, —CH₂CH₂NH—, —NHCH₂CH₂—, —C(═O)—, —C(═O)CH₂NH—, —CH₂C(═O)NH—, —C(═O)NH— or —NHC(═O)—;
each Cy1, Cy2, Cy3, Cy4 or Cy5 is independently selected from a bond or one of the following substituted or unsubstituted groups:

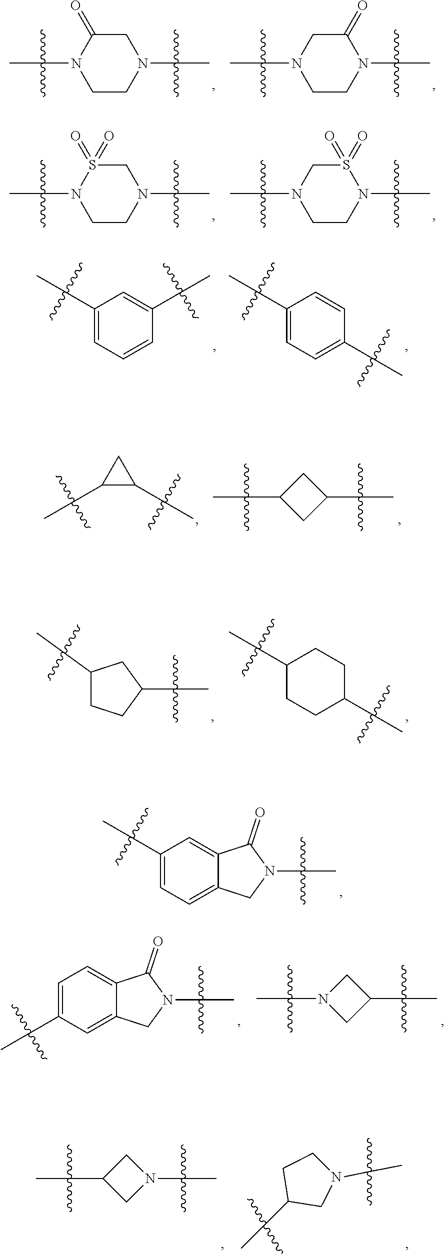

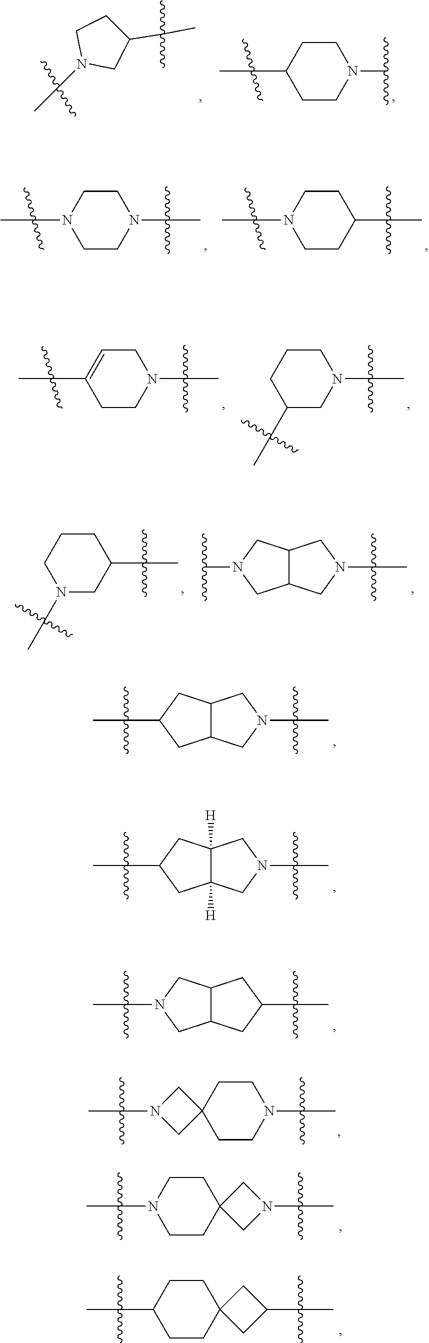

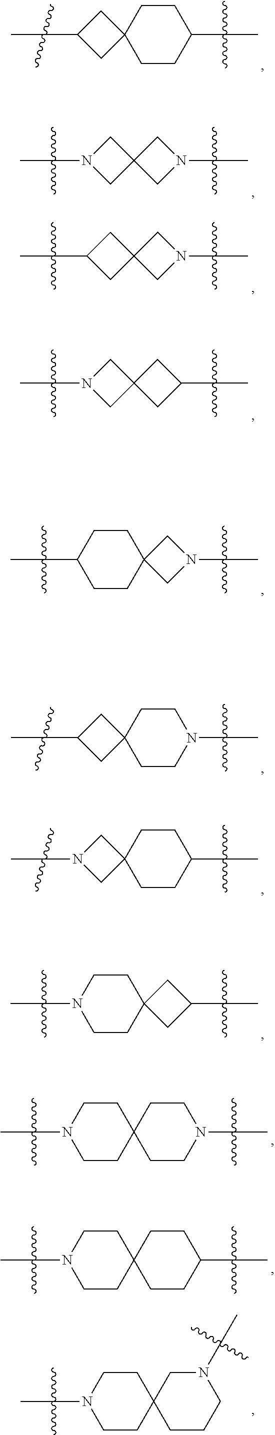

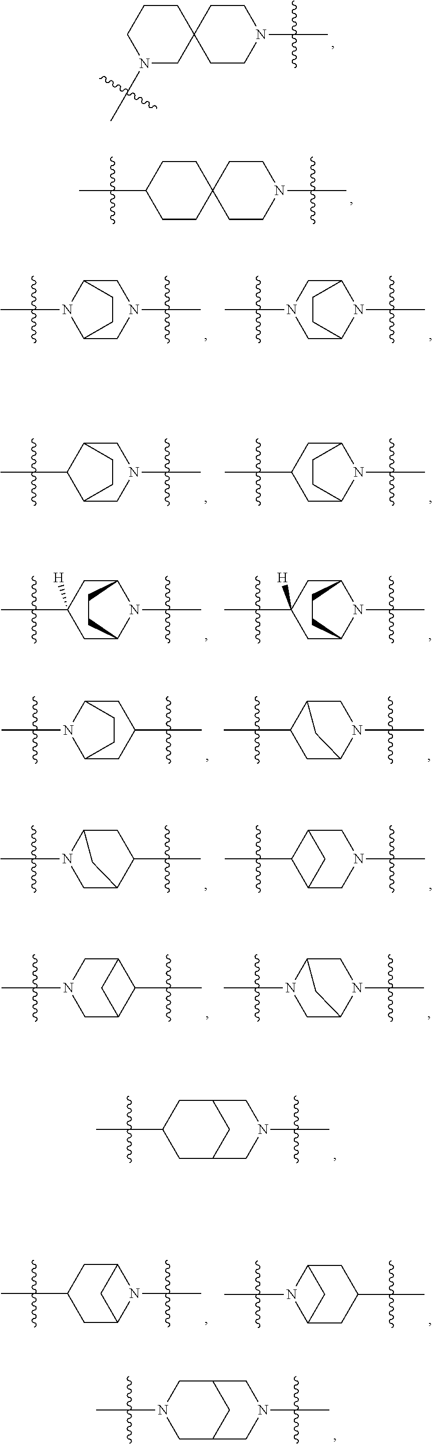

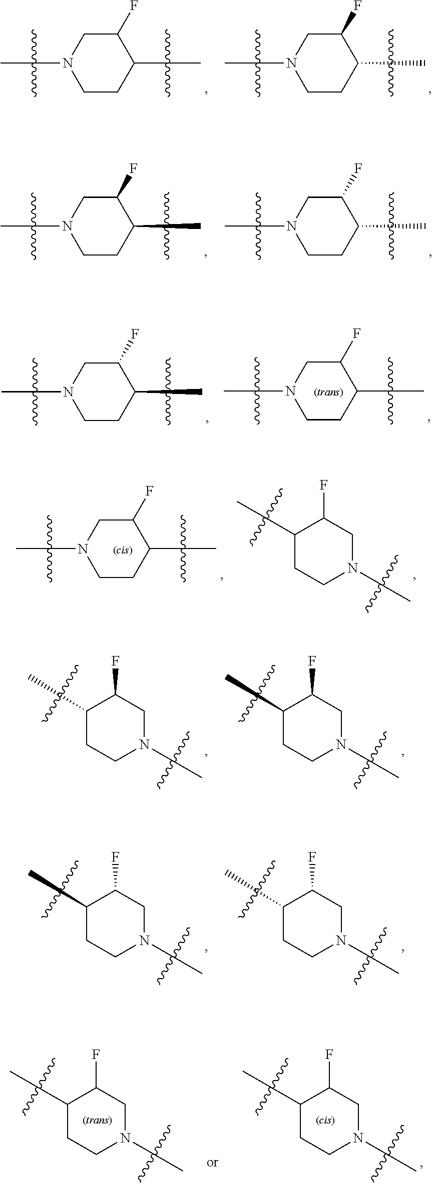

which, when substituted, is optionally further substituted with 0 to 4 substituents selected from H, F, CF₃, methyl, ═O, hydroxymethyl, COOH, CN or NH₂,
B is selected from one of the following structural fragments (Table B-a),

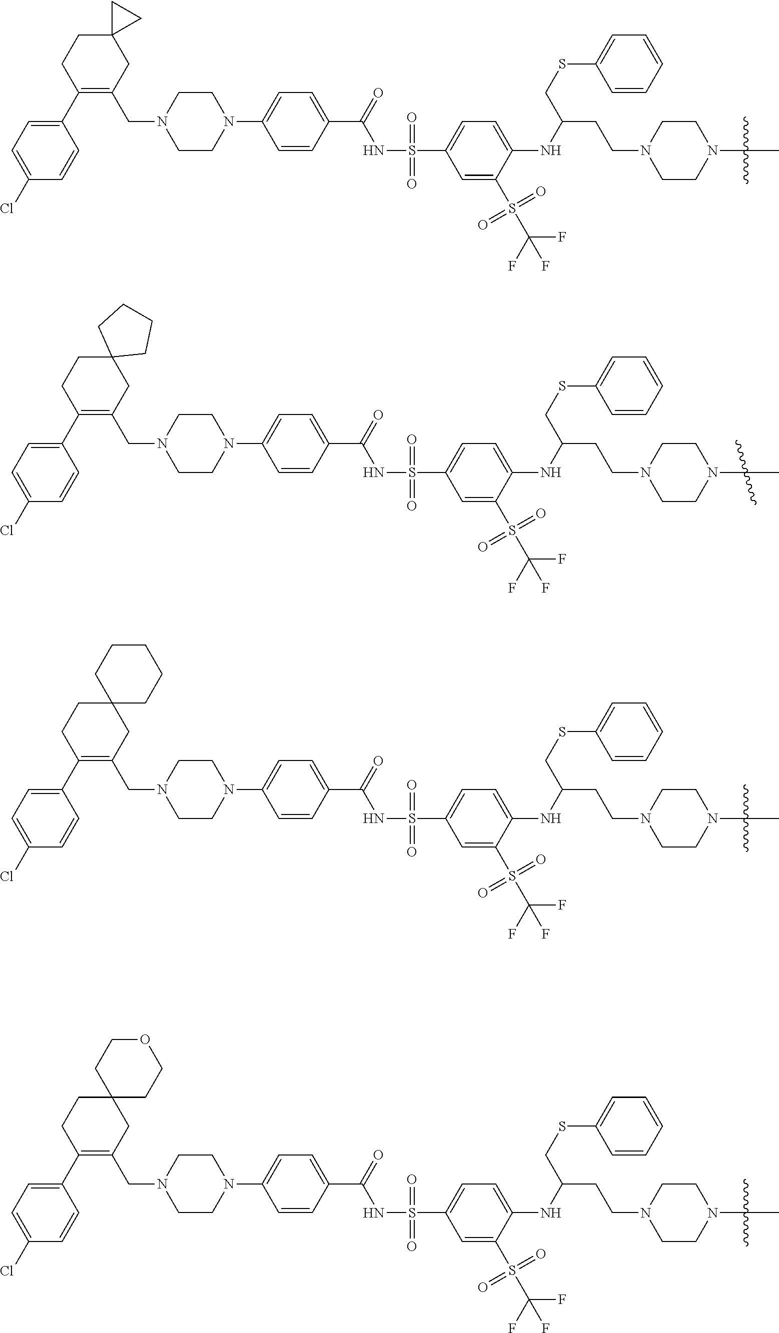 [SMILES: *N1CCN(CCC(CSc2ccccc2)Nc2ccc(S(=O)(=O)NC(=O)c3ccc(N4CCN(CC5=C(c6ccc(Cl)cc6)CCC6(CC6)C5)CC4)cc3)cc2S(=O)(=O)C(F)(F)F)CC1 ; *N1CCN(CCC(CSc2ccccc2)Nc2ccc(S(=O)(=O)NC(=O)c3ccc(N4CCN(CC5=C(c6ccc(Cl)cc6)CCC6(CCCC6)C5)CC4)cc3)cc2S(=O)(=O)C(F)(F)F)CC1 ; *N1CCN(CCC(CSc2ccccc2)Nc2ccc(S(=O)(=O)NC(=O)c3ccc(N4CCN(CC5=C(c6ccc(Cl)cc6)CCC6(CCCCC6)C5)CC4)cc3)cc2S(=O)(=O)C(F)(F)F)CC1 ; *N1CCN(CCC(CSc2ccccc2)Nc2ccc(S(=O)(=O)NC(=O)c3ccc(N4CCN(CC5=C(c6ccc(Cl)cc6)CCC6(CCOCC6)C5)CC4)cc3)cc2S(=O)(=O)C(F)(F)F)CC1]

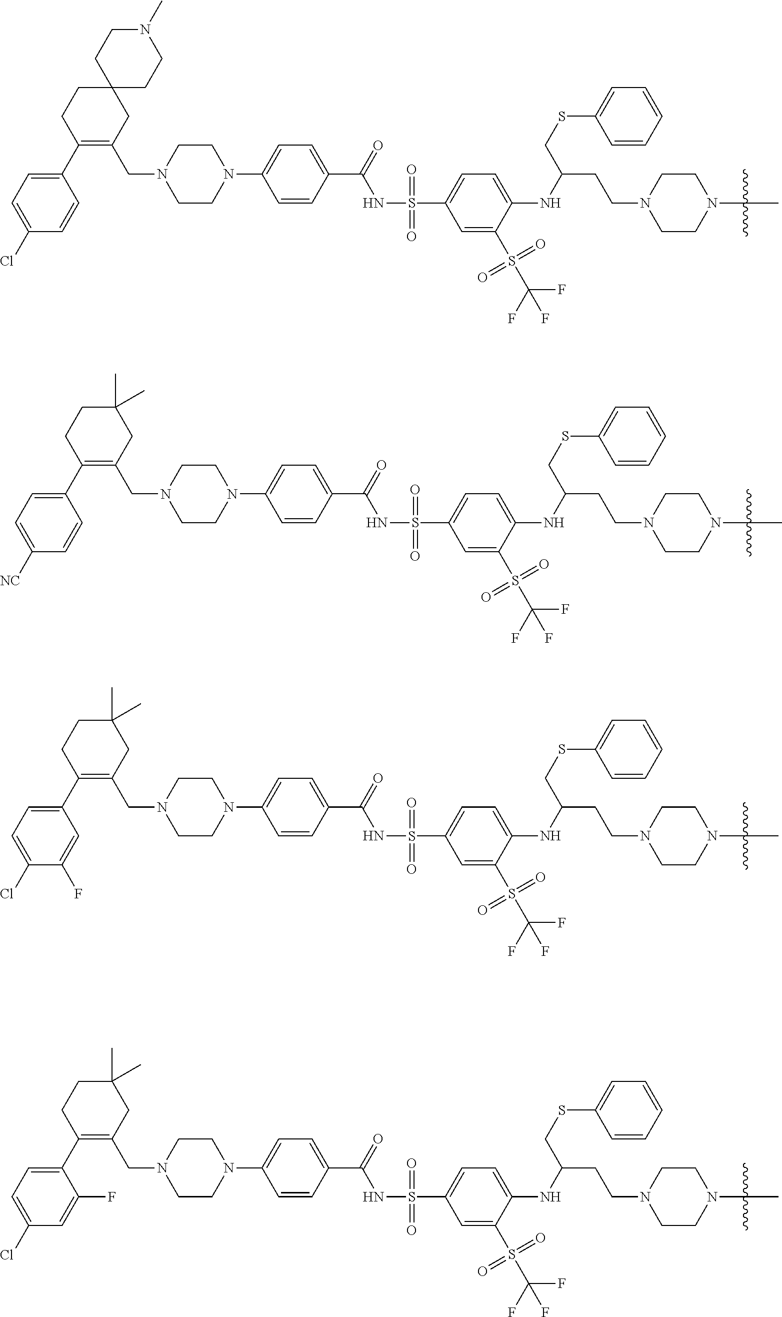 [SMILES: *N1CCN(CCC(CSc2ccccc2)Nc2ccc(S(=O)(=O)NC(=O)c3ccc(N4CCN(CC5=C(c6ccc(Cl)cc6)CCC6(CCN(C)CC6)C5)CC4)cc3)cc2S(=O)(=O)C(F)(F)F)CC1 ; *N1CCN(CCC(CSc2ccccc2)Nc2ccc(S(=O)(=O)NC(=O)c3ccc(N4CCN(CC5=C(c6ccc(C#N)cc6)CCC(C)(C)C5)CC4)cc3)cc2S(=O)(=O)C(F)(F)F)CC1 ; *N1CCN(CCC(CSc2ccccc2)Nc2ccc(S(=O)(=O)NC(=O)c3ccc(N4CCN(CC5=C(c6ccc(Cl)c(F)c6)CCC(C)(C)C5)CC4)cc3)cc2S(=O)(=O)C(F)(F)F)CC1 ; *N1CCN(CCC(CSc2ccccc2)Nc2ccc(S(=O)(=O)NC(=O)c3ccc(N4CCN(CC5=C(c6ccc(Cl)cc6F)CCC(C)(C)C5)CC4)cc3)cc2S(=O)(=O)C(F)(F)F)CC1]

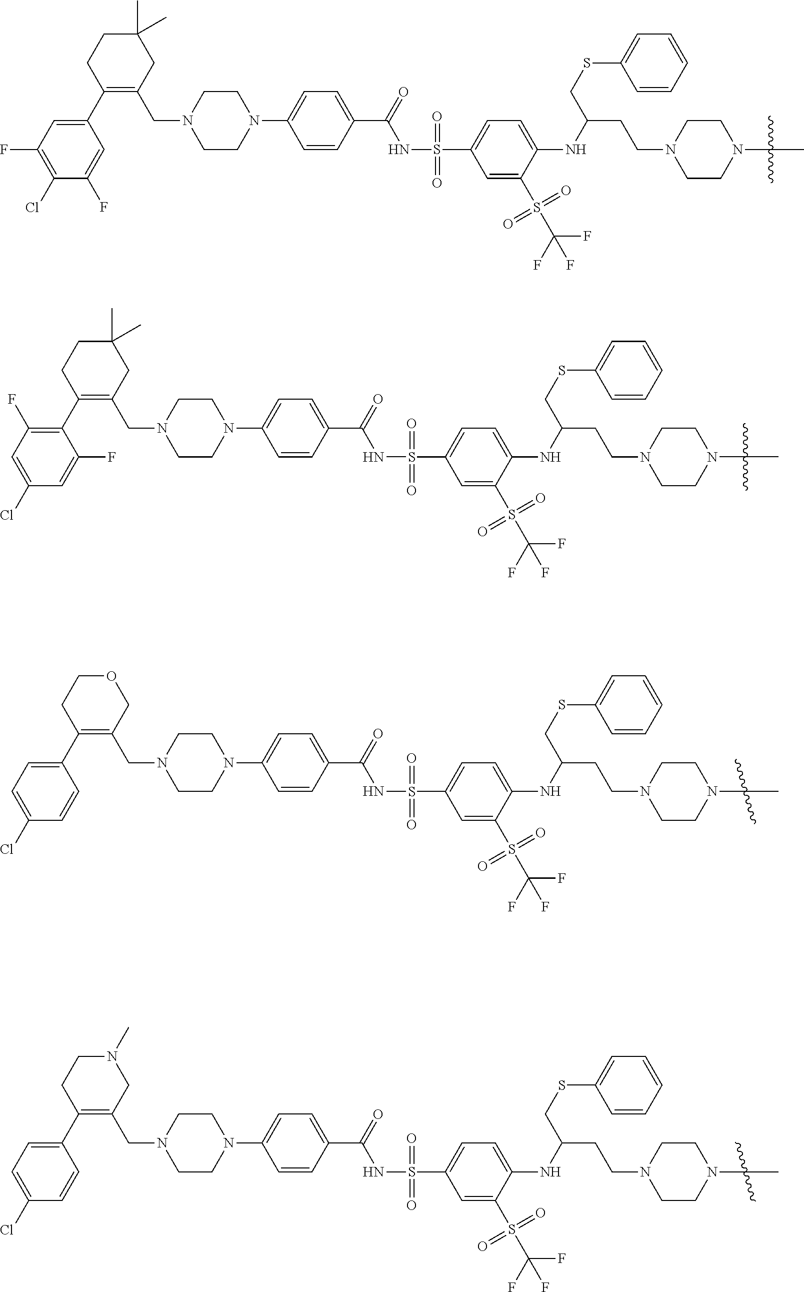 [SMILES: *N1CCN(CCC(CSc2ccccc2)Nc2ccc(S(=O)(=O)NC(=O)c3ccc(N4CCN(CC5=C(c6cc(F)c(Cl)c(F)c6)CCC(C)(C)C5)CC4)cc3)cc2S(=O)(=O)C(F)(F)F)CC1 ; *N1CCN(CCC(CSc2ccccc2)Nc2ccc(S(=O)(=O)NC(=O)c3ccc(N4CCN(CC5=C(c6c(F)cc(Cl)cc6F)CCC(C)(C)C5)CC4)cc3)cc2S(=O)(=O)C(F)(F)F)CC1 ; *N1CCN(CCC(CSc2ccccc2)Nc2ccc(S(=O)(=O)NC(=O)c3ccc(N4CCN(CC5=C(c6ccc(Cl)cc6)CCOC5)CC4)cc3)cc2S(=O)(=O)C(F)(F)F)CC1 ; *N1CCN(CCC(CSc2ccccc2)Nc2ccc(S(=O)(=O)NC(=O)c3ccc(N4CCN(CC5=C(c6ccc(Cl)cc6)CCN(C)C5)CC4)cc3)cc2S(=O)(=O)C(F)(F)F)CC1]

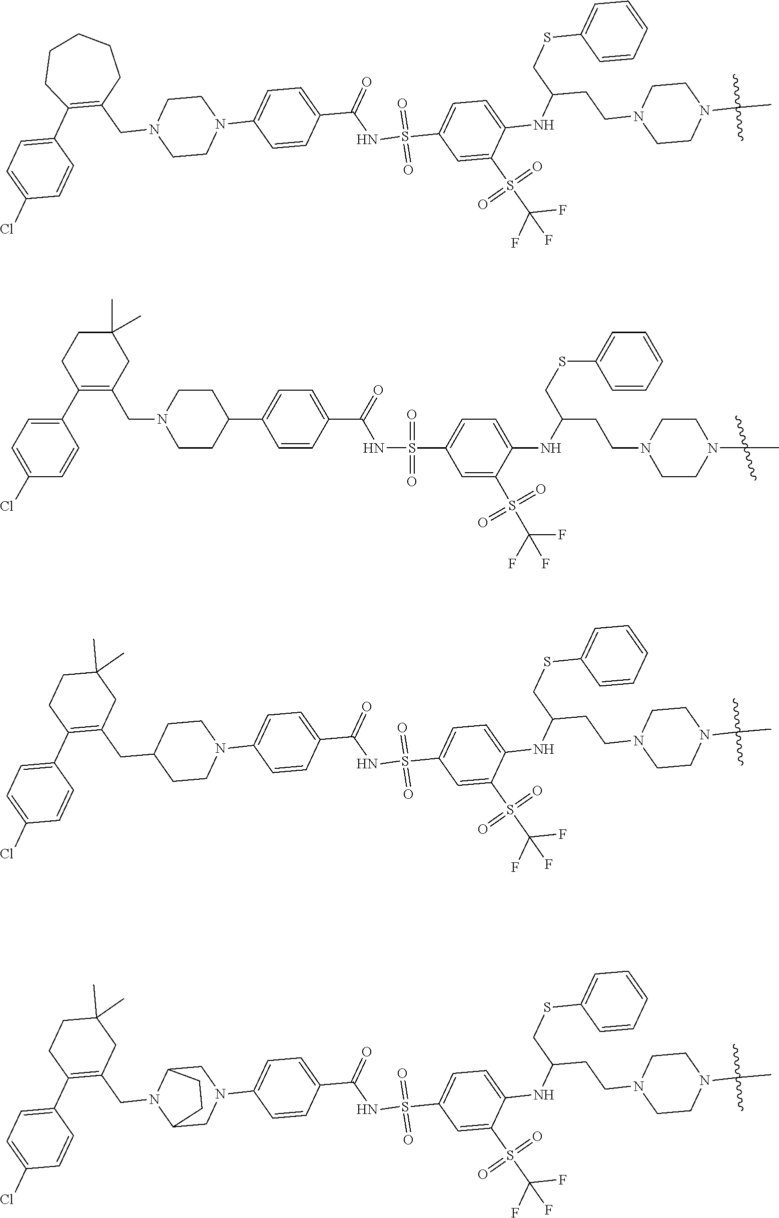 [SMILES: *N1CCN(CCC(CSc2ccccc2)Nc2ccc(S(=O)(=O)NC(=O)c3ccc(N4CCN(CC5=C(c6ccc(Cl)cc6)CCCCC5)CC4)cc3)cc2S(=O)(=O)C(F)(F)F)CC1 ; *N1CCN(CCC(CSc2ccccc2)Nc2ccc(S(=O)(=O)NC(=O)c3ccc(C4CCN(CC5=C(c6ccc(Cl)cc6)CCC(C)(C)C5)CC4)cc3)cc2S(=O)(=O)C(F)(F)F)CC1 ; *N1CCN(CCC(CSc2ccccc2)Nc2ccc(S(=O)(=O)NC(=O)c3ccc(N4CCC(CC5=C(c6ccc(Cl)cc6)CCC(C)(C)C5)CC4)cc3)cc2S(=O)(=O)C(F)(F)F)CC1 ; *N1CCN(CCC(CSc2ccccc2)Nc2ccc(S(=O)(=O)NC(=O)c3ccc(N4CC5CCC(C4)N5CC4=C(c5ccc(Cl)cc5)CCC(C)(C)C4)cc3)cc2S(=O)(=O)C(F)(F)F)CC1]

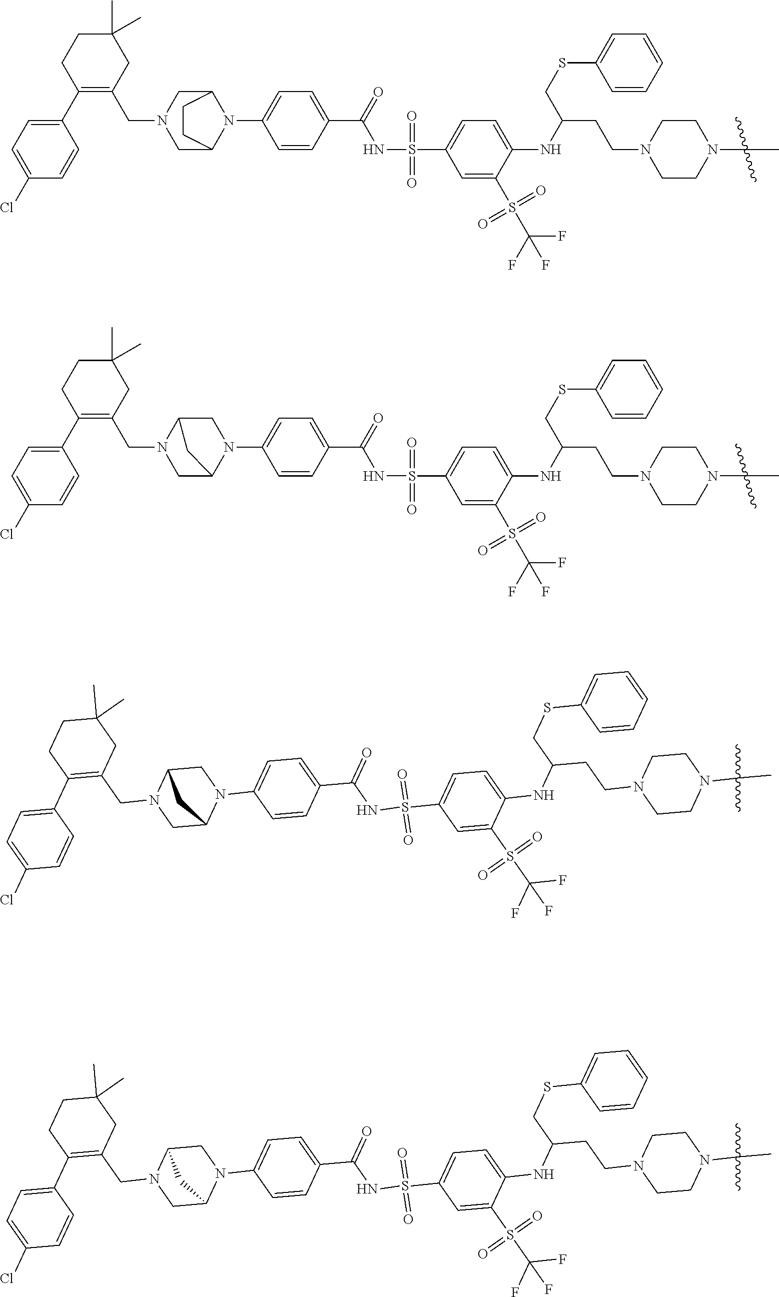

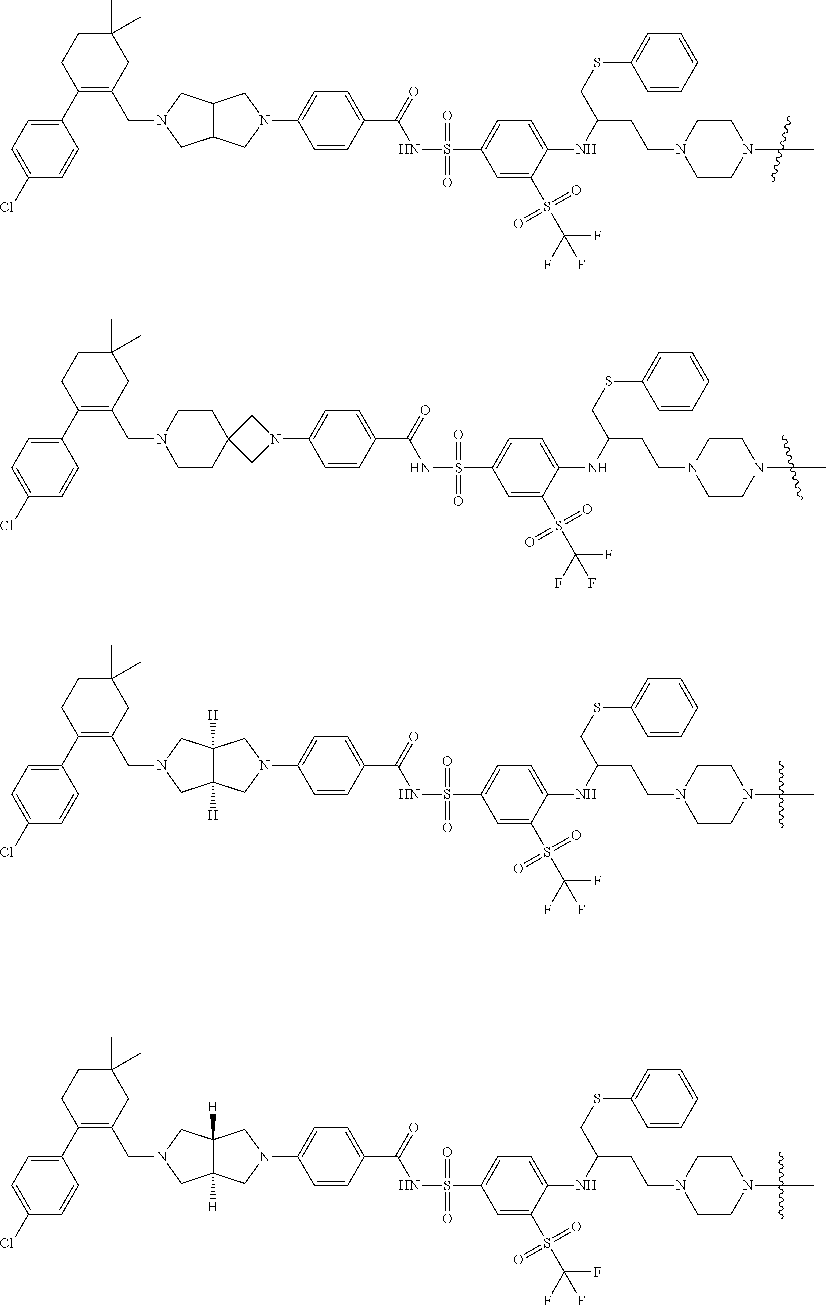

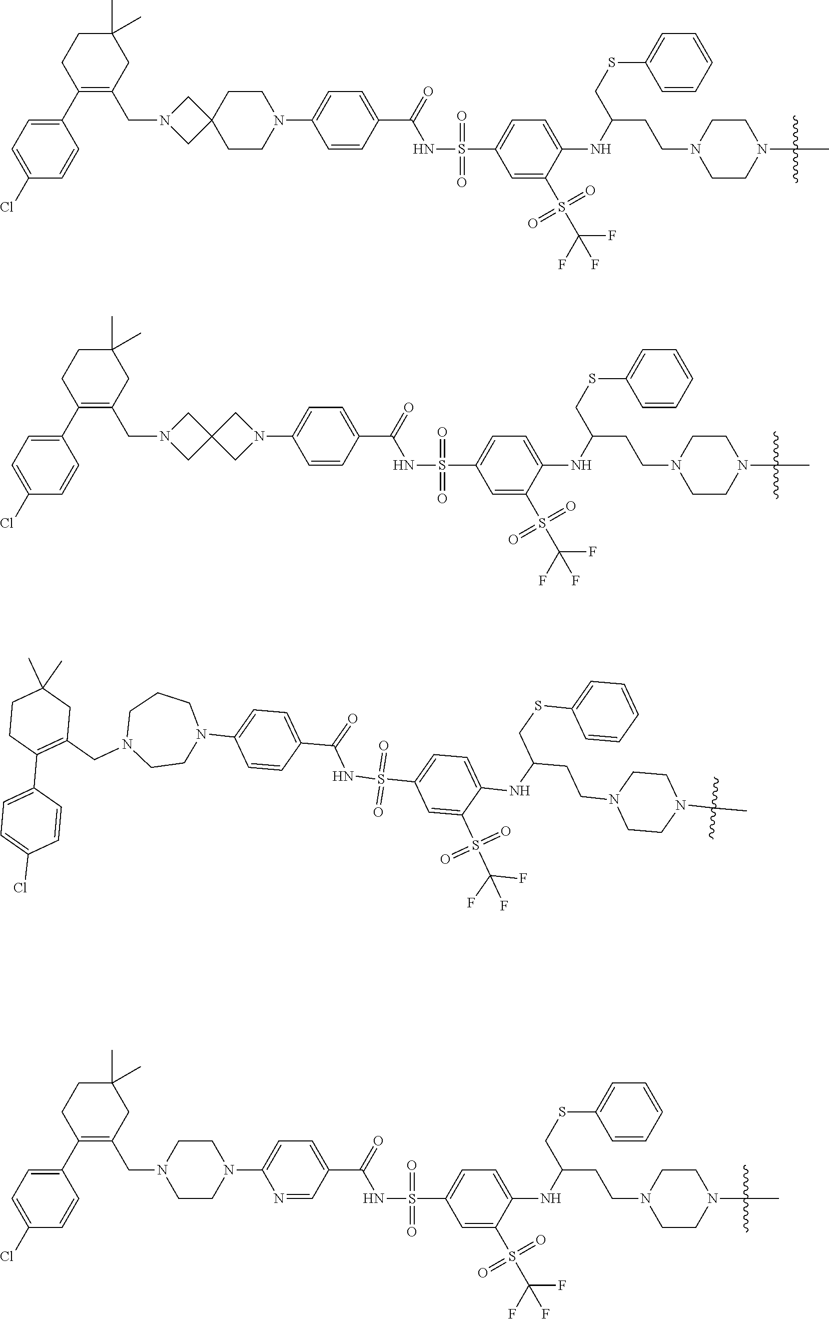 [SMILES: *N1CCN(CCC(CSc2ccccc2)Nc2ccc(S(=O)(=O)NC(=O)c3ccc(N4CCC5(CC4)CN(CC4=C(c6ccc(Cl)cc6)CCC(C)(C)C4)C5)cc3)cc2S(=O)(=O)C(F)(F)F)CC1 ; *N1CCN(CCC(CSc2ccccc2)Nc2ccc(S(=O)(=O)NC(=O)c3ccc(N4CC5(CN(CC6=C(c7ccc(Cl)cc7)CCC(C)(C)C6)C5)C4)cc3)cc2S(=O)(=O)C(F)(F)F)CC1 ; *N1CCN(CCC(CSc2ccccc2)Nc2ccc(S(=O)(=O)NC(=O)c3ccc(N4CCCN(CC5=C(c6ccc(Cl)cc6)CCC(C)(C)C5)CC4)cc3)cc2S(=O)(=O)C(F)(F)F)CC1 ; *N1CCN(CCC(CSc2ccccc2)Nc2ccc(S(=O)(=O)NC(=O)c3ccc(N4CCN(CC5=C(c6ccc(Cl)cc6)CCC(C)(C)C5)CC4)nc3)cc2S(=O)(=O)C(F)(F)F)CC1]

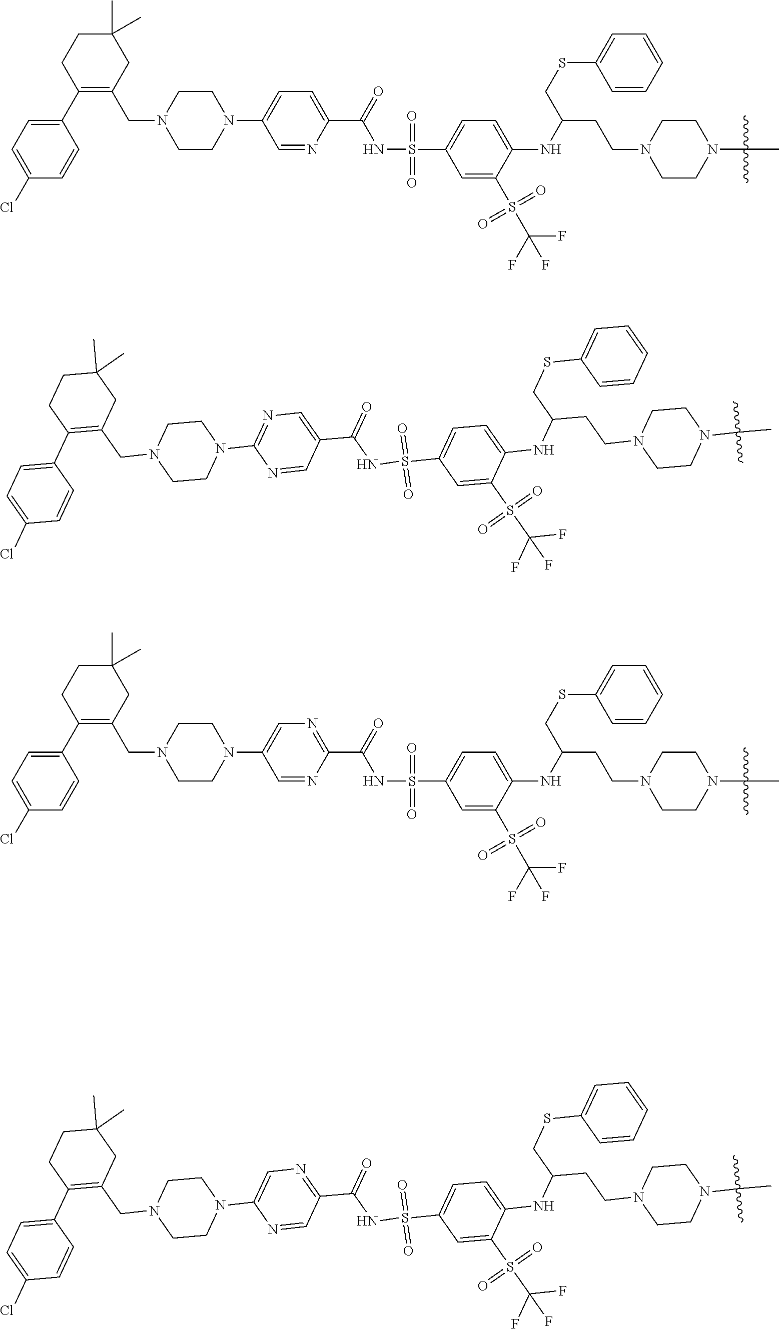 [SMILES: *N1CCN(CCC(CSc2ccccc2)Nc2ccc(S(=O)(=O)NC(=O)c3ccc(N4CCN(CC5=C(c6ccc(Cl)cc6)CCC(C)(C)C5)CC4)cn3)cc2S(=O)(=O)C(F)(F)F)CC1 ; *N1CCN(CCC(CSc2ccccc2)Nc2ccc(S(=O)(=O)NC(=O)c3cnc(N4CCN(CC5=C(c6ccc(Cl)cc6)CCC(C)(C)C5)CC4)nc3)cc2S(=O)(=O)C(F)(F)F)CC1 ; *N1CCN(CCC(CSc2ccccc2)Nc2ccc(S(=O)(=O)NC(=O)c3ncc(N4CCN(CC5=C(c6ccc(Cl)cc6)CCC(C)(C)C5)CC4)cn3)cc2S(=O)(=O)C(F)(F)F)CC1 ; *N1CCN(CCC(CSc2ccccc2)Nc2ccc(S(=O)(=O)NC(=O)c3cnc(N4CCN(CC5=C(c6ccc(Cl)cc6)CCC(C)(C)C5)CC4)cn3)cc2S(=O)(=O)C(F)(F)F)CC1]

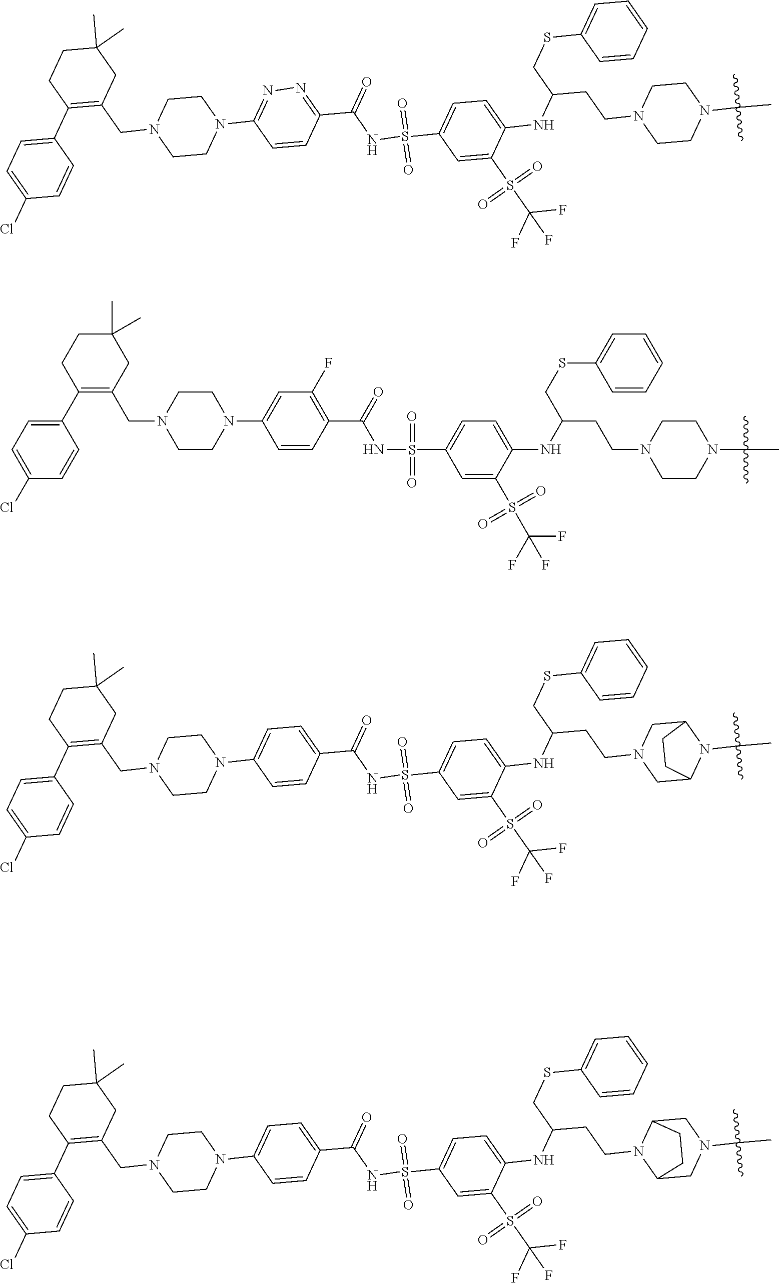 [SMILES: *N1CCN(CCC(CSc2ccccc2)Nc2ccc(S(=O)(=O)NC(=O)c3ccc(N4CCN(CC5=C(c6ccc(Cl)cc6)CCC(C)(C)C5)CC4)nn3)cc2S(=O)(=O)C(F)(F)F)CC1 ; *N1CCN(CCC(CSc2ccccc2)Nc2ccc(S(=O)(=O)NC(=O)c3ccc(N4CCN(CC5=C(c6ccc(Cl)cc6)CCC(C)(C)C5)CC4)cc3F)cc2S(=O)(=O)C(F)(F)F)CC1 ; *N1C2CCC1CN(CCC(CSc1ccccc1)Nc1ccc(S(=O)(=O)NC(=O)c3ccc(N4CCN(CC5=C(c6ccc(Cl)cc6)CCC(C)(C)C5)CC4)cc3)cc1S(=O)(=O)C(F)(F)F)C2 ; *N1CC2CCC(C1)N2CCC(CSc1ccccc1)Nc1ccc(S(=O)(=O)NC(=O)c2ccc(N3CCN(CC4=C(c5ccc(Cl)cc5)CCC(C)(C)C4)CC3)cc2)cc1S(=O)(=O)C(F)(F)F]

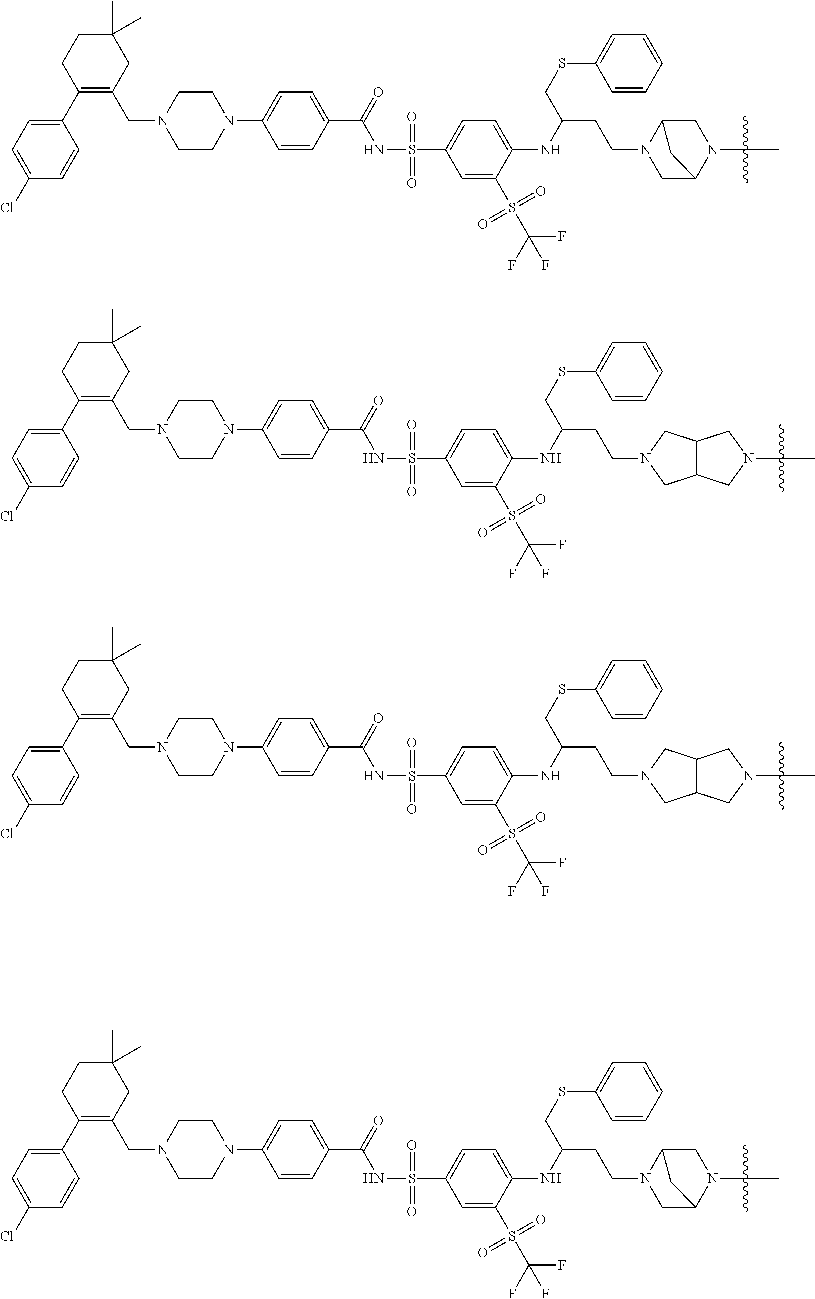 [SMILES: *N1CC2CC1CN2CCC(CSc1ccccc1)Nc1ccc(S(=O)(=O)NC(=O)c2ccc(N3CCN(CC4=C(c5ccc(Cl)cc5)CCC(C)(C)C4)CC3)cc2)cc1S(=O)(=O)C(F)(F)F ; *N1CC2CN(CCC(CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2C1 ; *N1CC2CN(CCC(CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2C1 ; *N1CC2CC1CN2CCC(CSc1ccccc1)Nc1ccc(S(=O)(=O)NC(=O)c2ccc(N3CCN(CC4=C(c5ccc(Cl)cc5)CCC(C)(C)C4)CC3)cc2)cc1S(=O)(=O)C(F)(F)F]

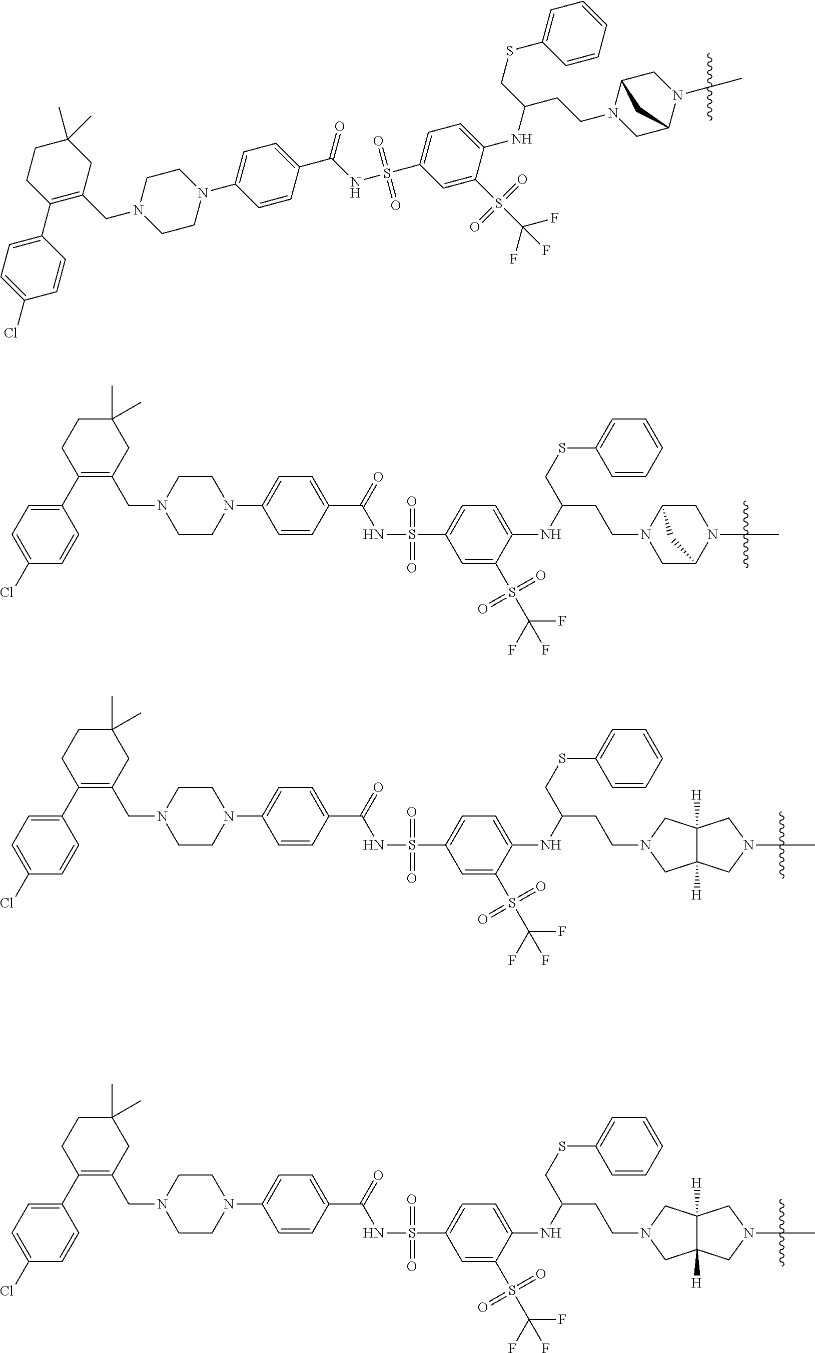

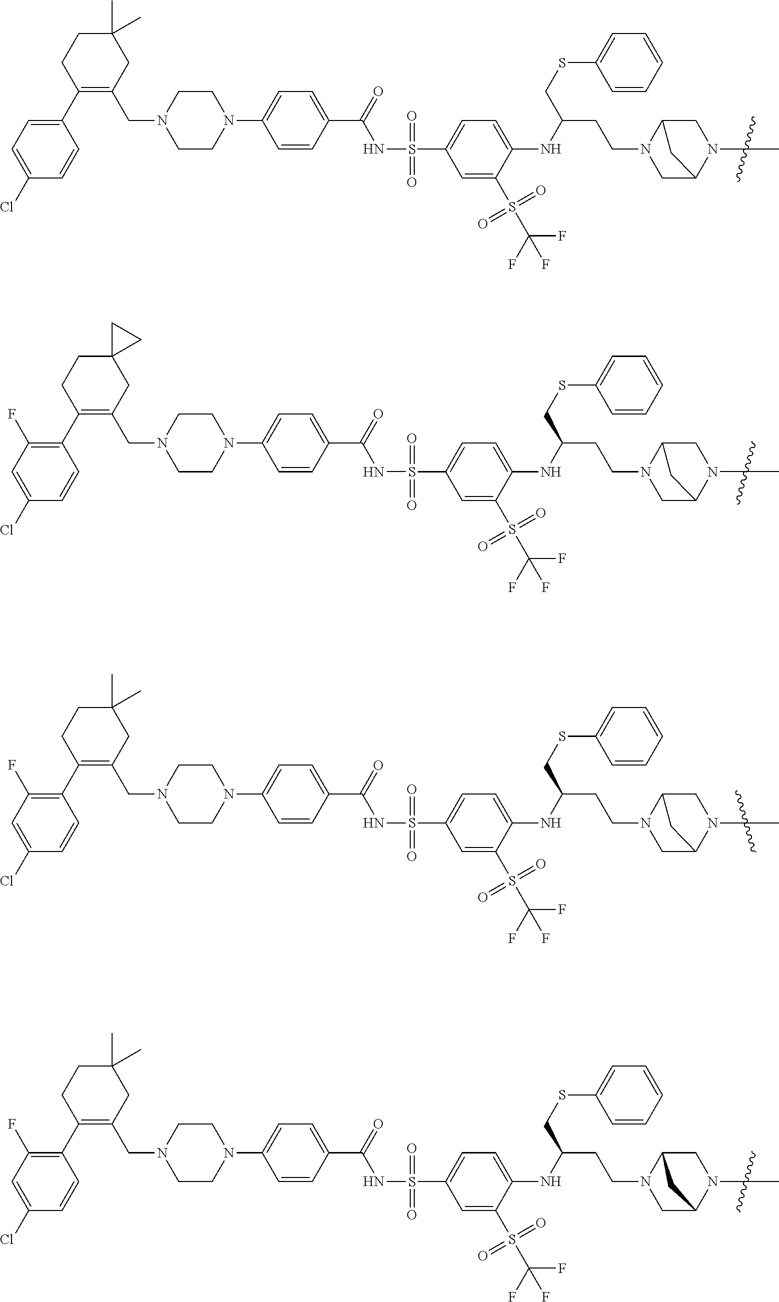

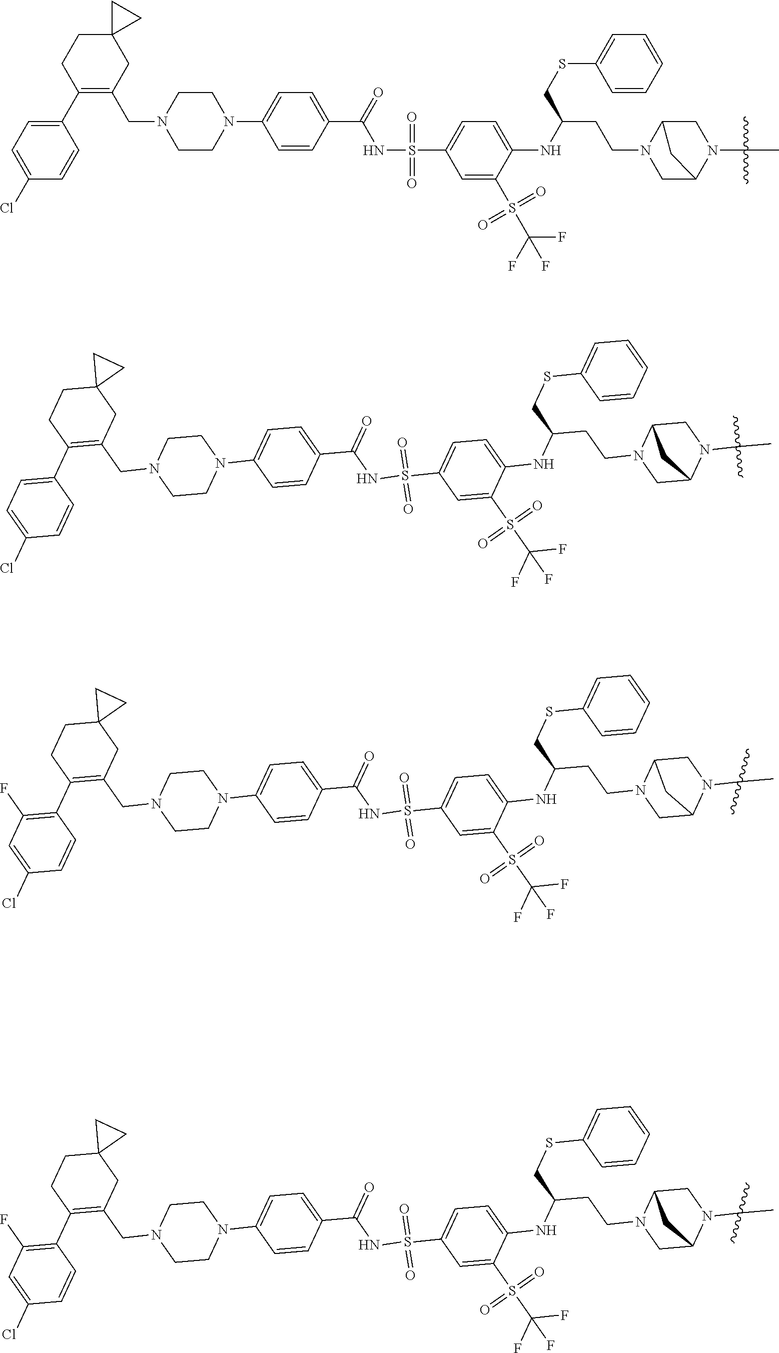

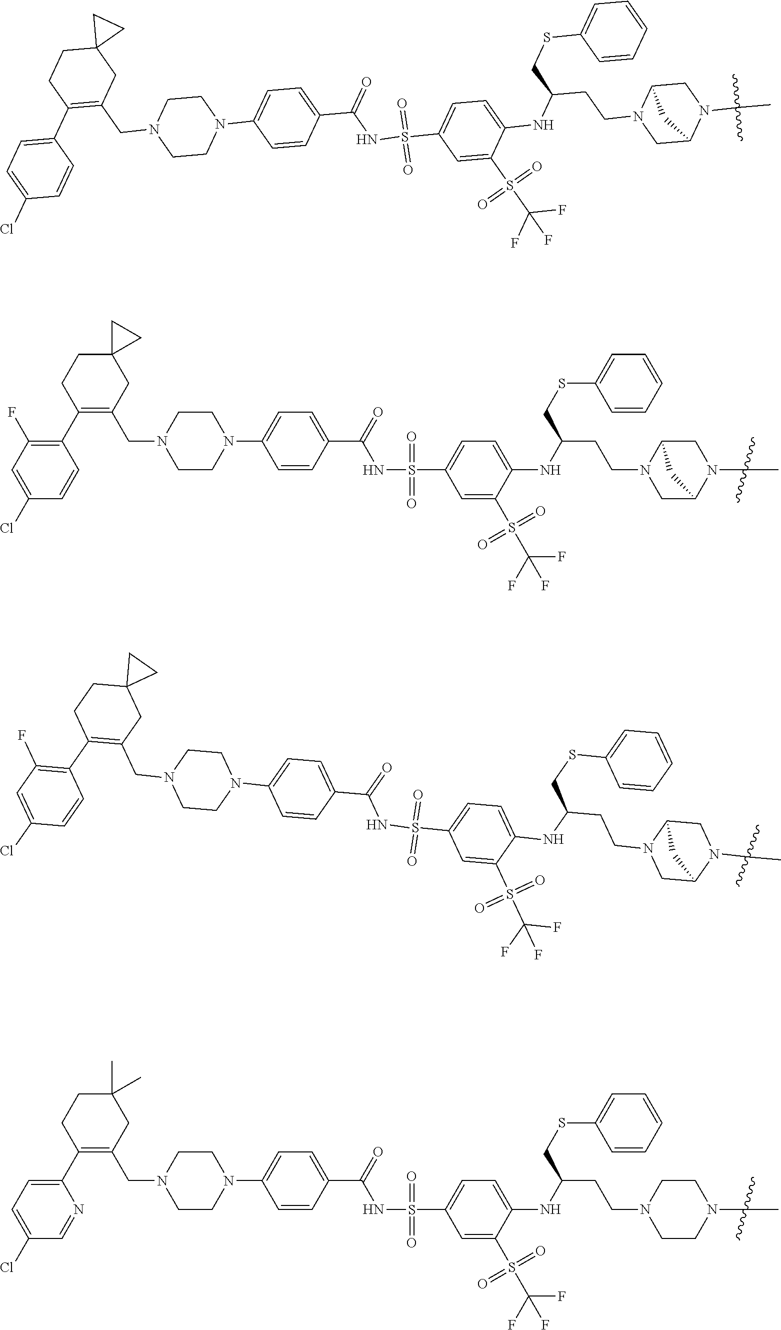

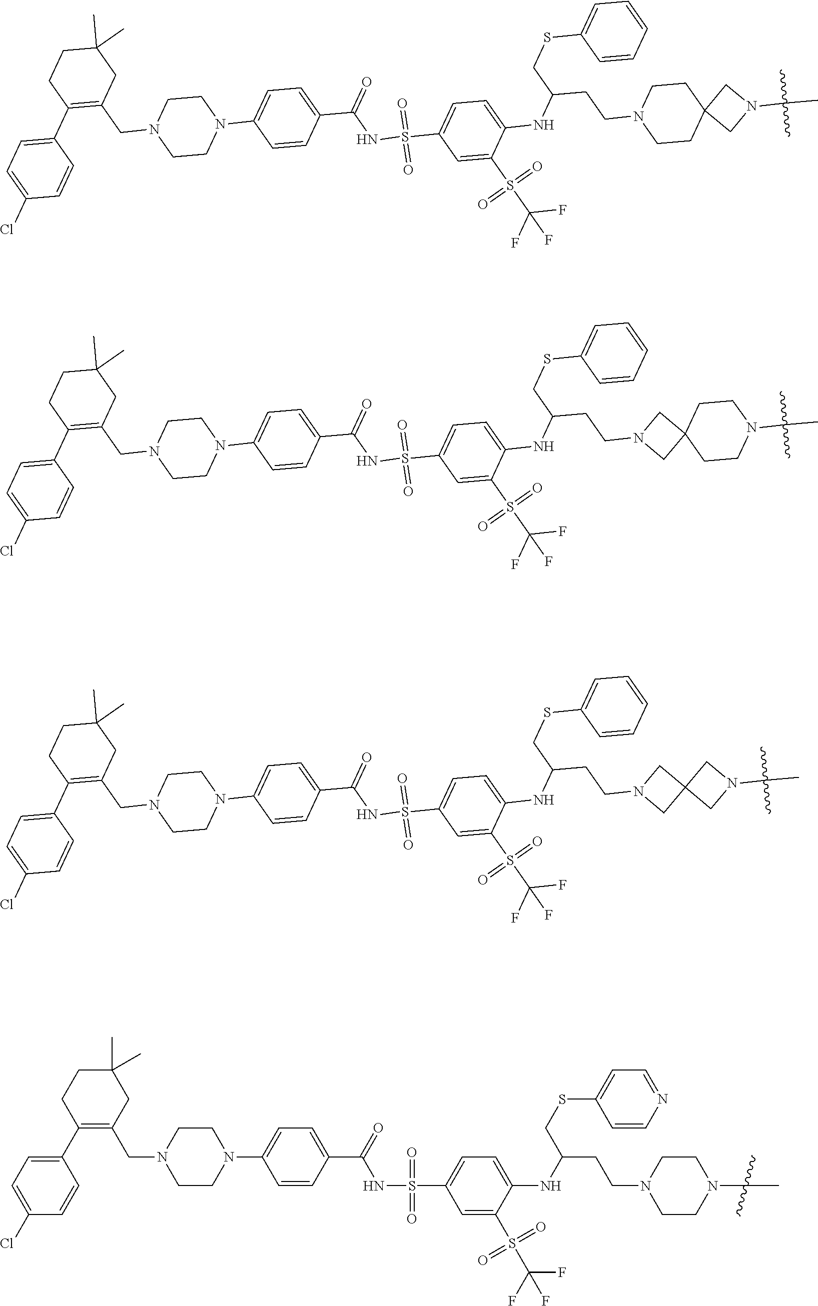

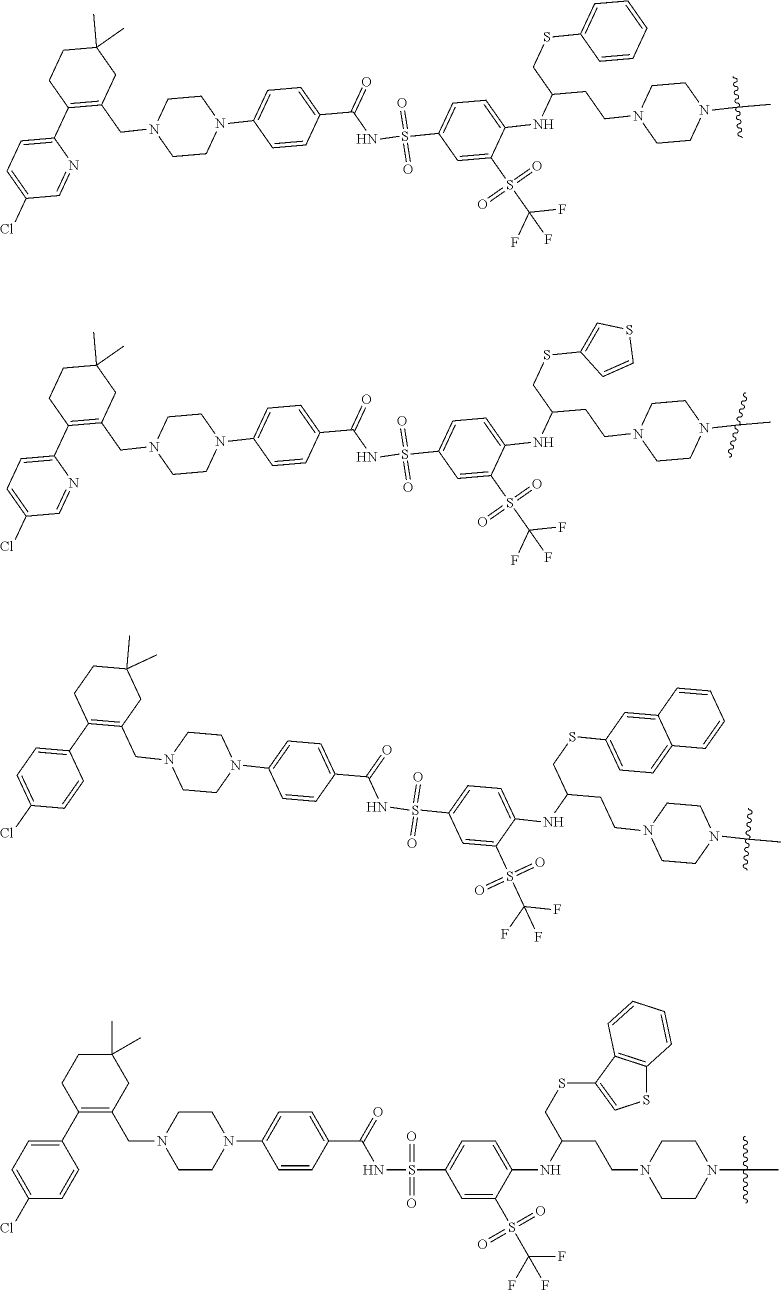 [SMILES: *N1CCN(CCC(CSc2ccccc2)Nc2ccc(S(=O)(=O)NC(=O)c3ccc(N4CCN(CC5=C(c6ccc(Cl)cn6)CCC(C)(C)C5)CC4)cc3)cc2S(=O)(=O)C(F)(F)F)CC1 ; *N1CCN(CCC(CSc2ccsc2)Nc2ccc(S(=O)(=O)NC(=O)c3ccc(N4CCN(CC5=C(c6ccc(Cl)cn6)CCC(C)(C)C5)CC4)cc3)cc2S(=O)(=O)C(F)(F)F)CC1 ; *N1CCN(CCC(CSc2ccc3ccccc3c2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)CC1 ; *N1CCN(CCC(CSc2csc3ccccc23)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)CC1]

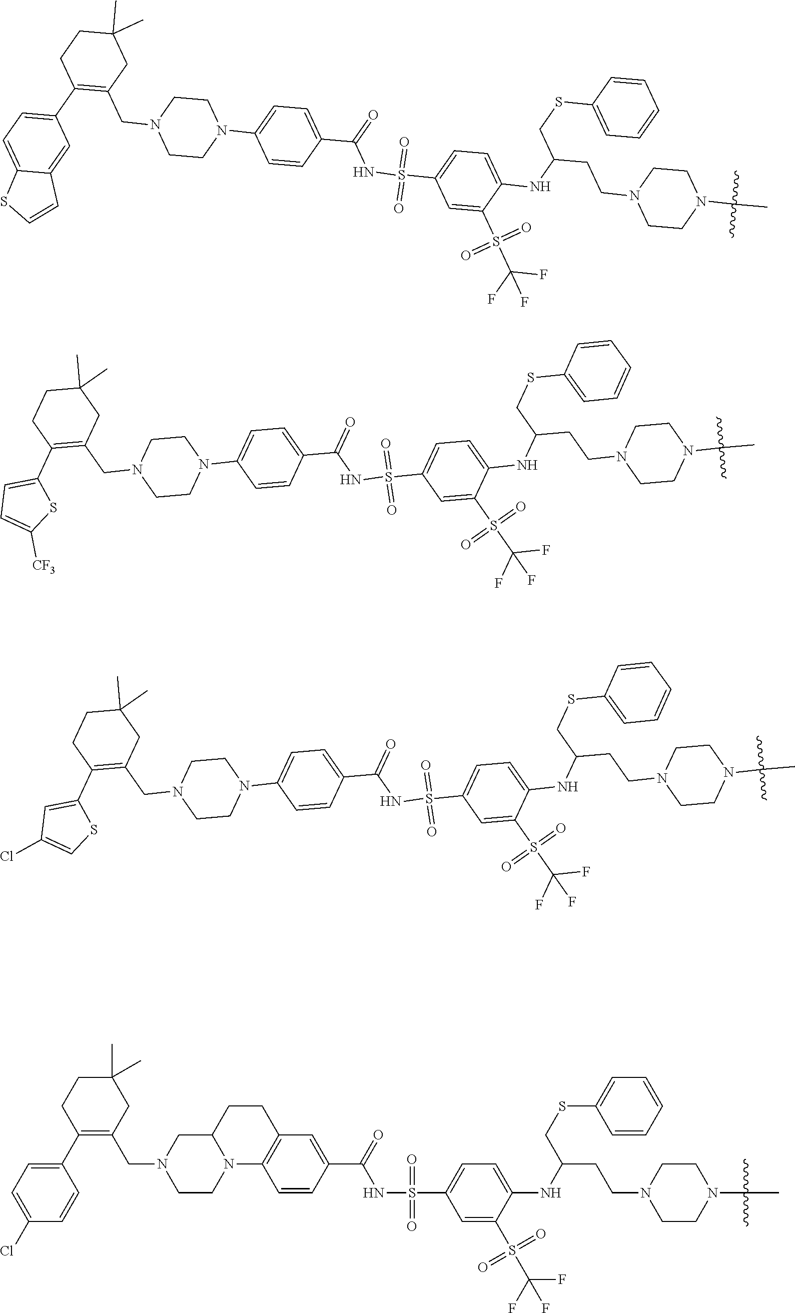 [SMILES: *N1CCN(CCC(CSc2ccccc2)Nc2ccc(S(=O)(=O)NC(=O)c3ccc(N4CCN(CC5=C(c6ccc7sccc7c6)CCC(C)(C)C5)CC4)cc3)cc2S(=O)(=O)C(F)(F)F)CC1 ; *N1CCN(CCC(CSc2ccccc2)Nc2ccc(S(=O)(=O)NC(=O)c3ccc(N4CCN(CC5=C(c6ccc(C(F)(F)F)s6)CCC(C)(C)C5)CC4)cc3)cc2S(=O)(=O)C(F)(F)F)CC1 ; *N1CCN(CCC(CSc2ccccc2)Nc2ccc(S(=O)(=O)NC(=O)c3ccc(N4CCN(CC5=C(c6cc(Cl)cs6)CCC(C)(C)C5)CC4)cc3)cc2S(=O)(=O)C(F)(F)F)CC1 ; *N1CCN(CCC(CSc2ccccc2)Nc2ccc(S(=O)(=O)NC(=O)c3ccc4c(c3)CCC3CN(CC5=C(c6ccc(Cl)cc6)CCC(C)(C)C5)CCN43)cc2S(=O)(=O)C(F)(F)F)CC1]

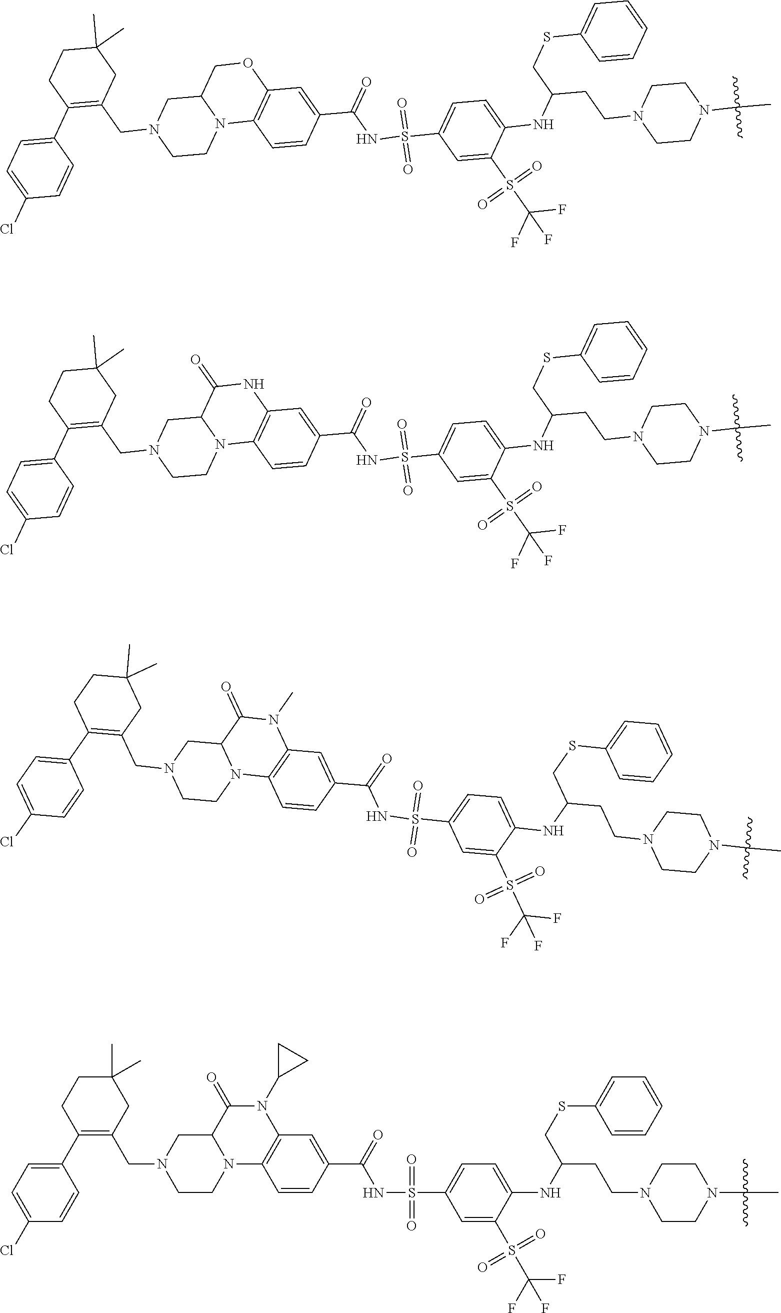

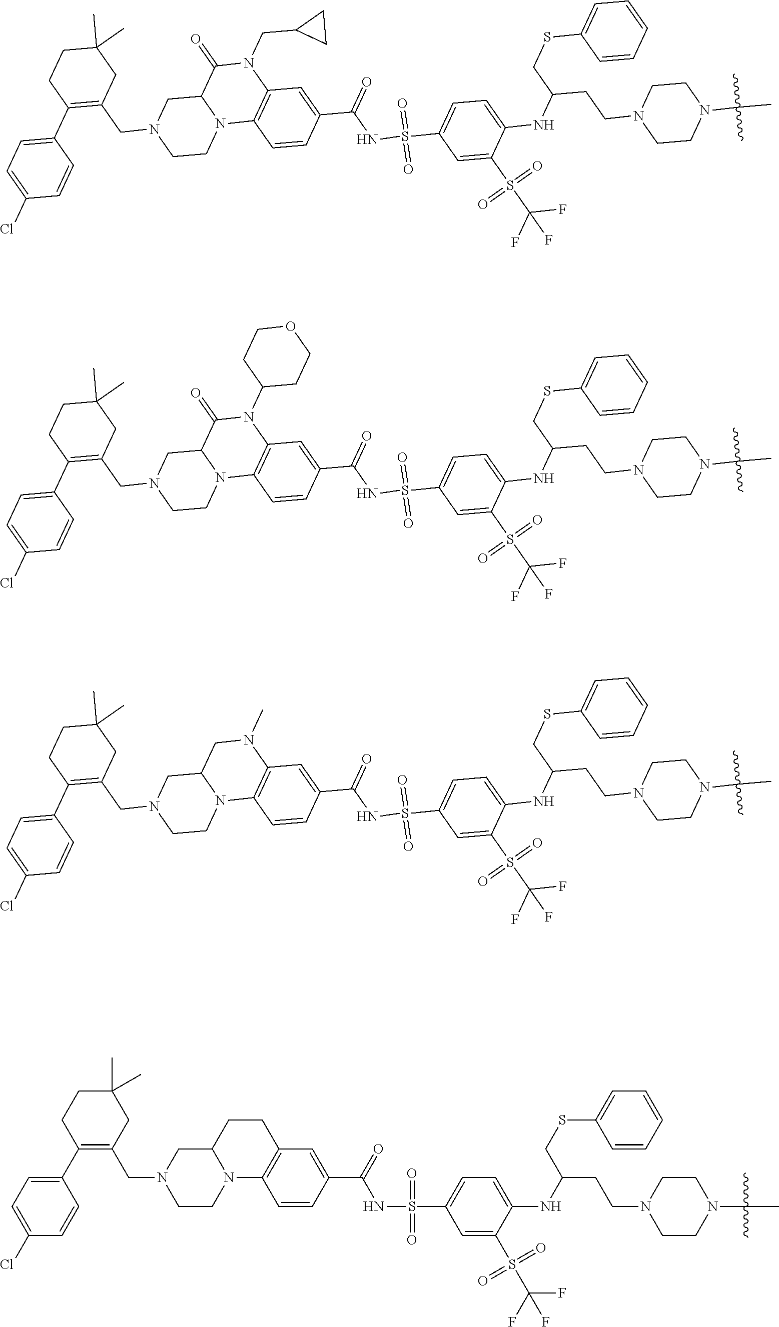

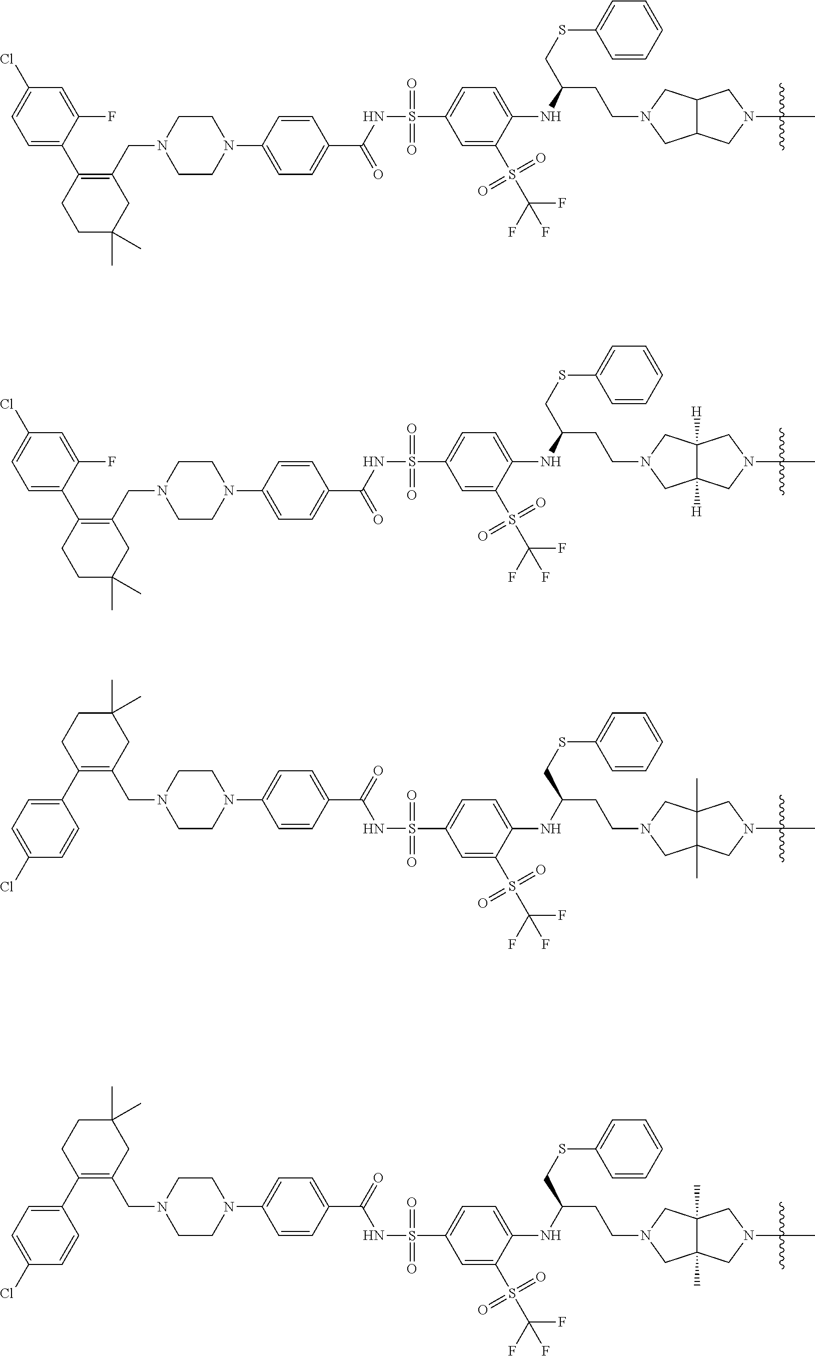

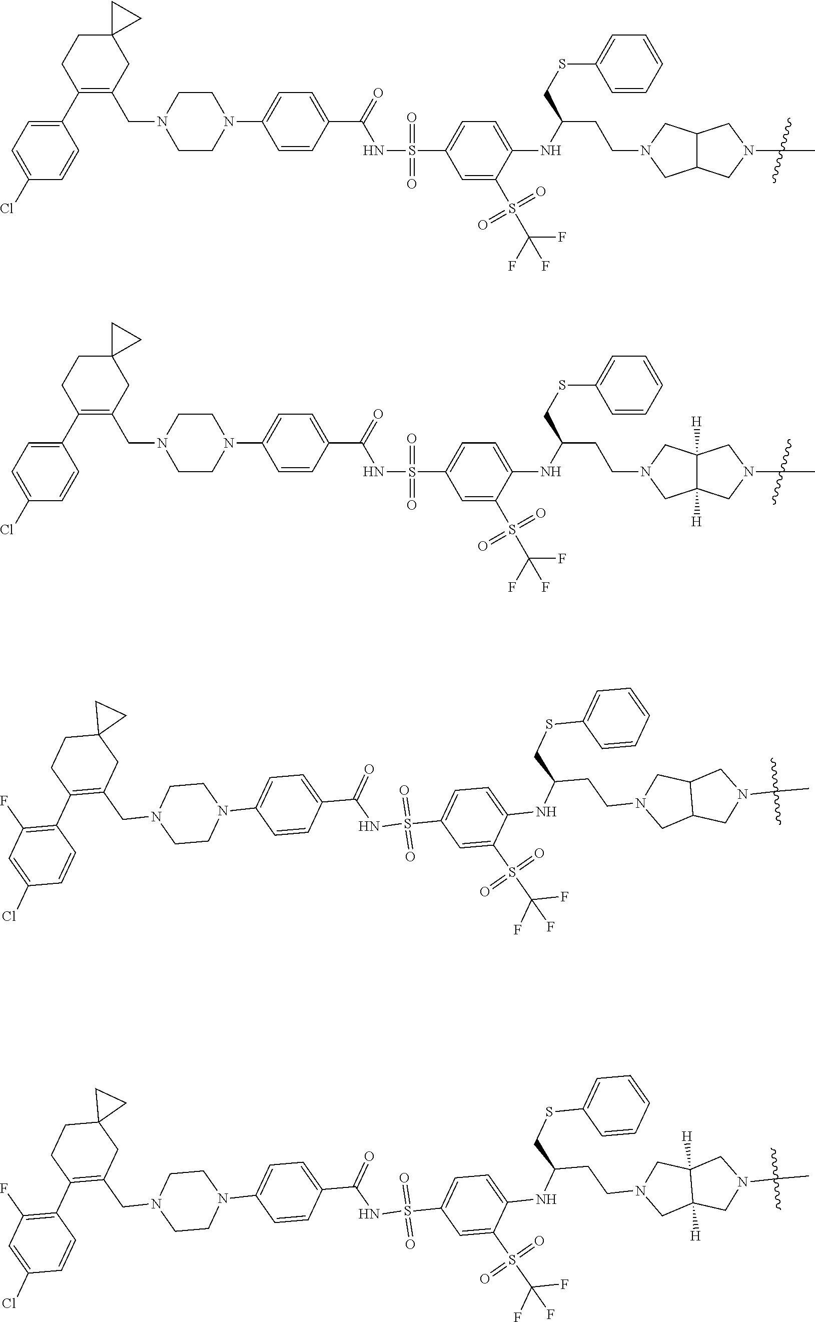

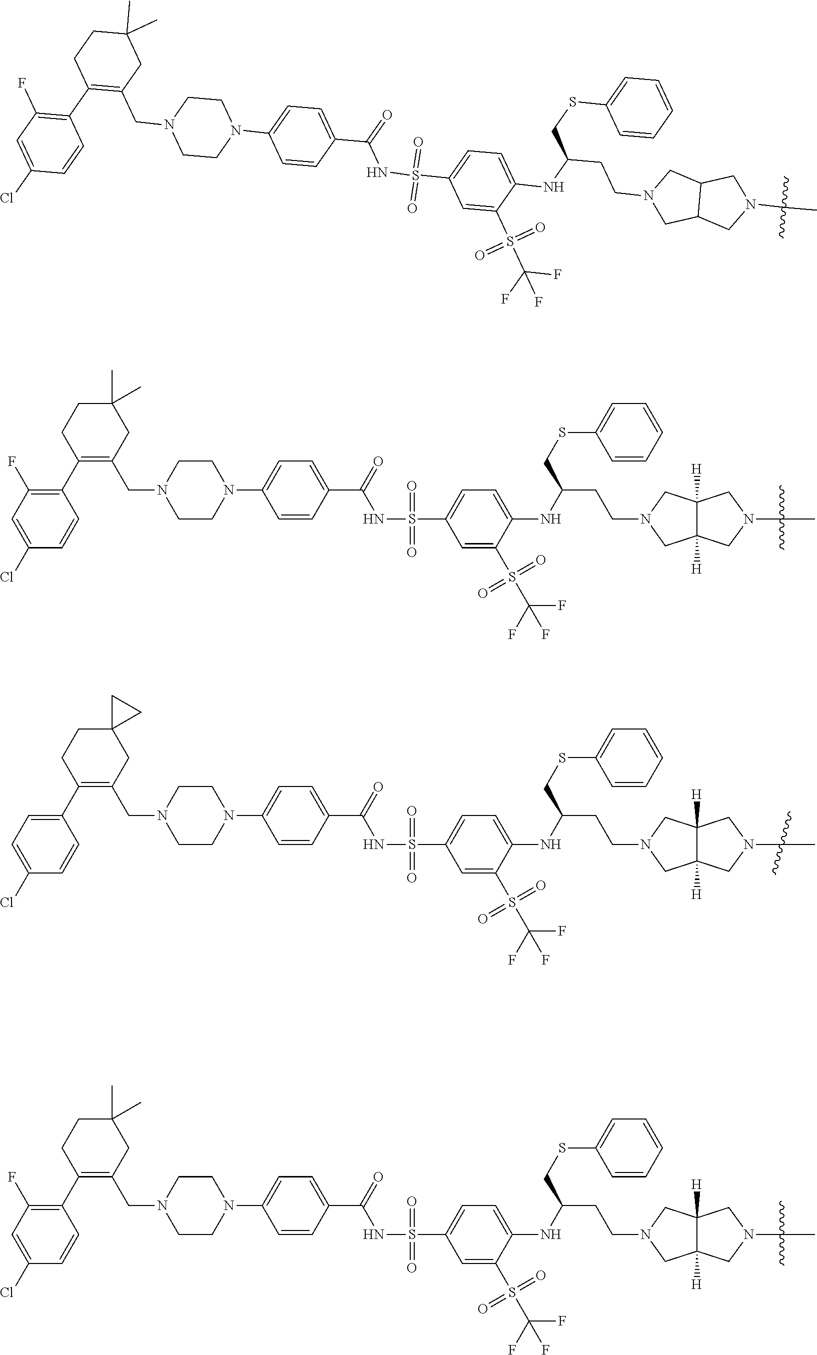

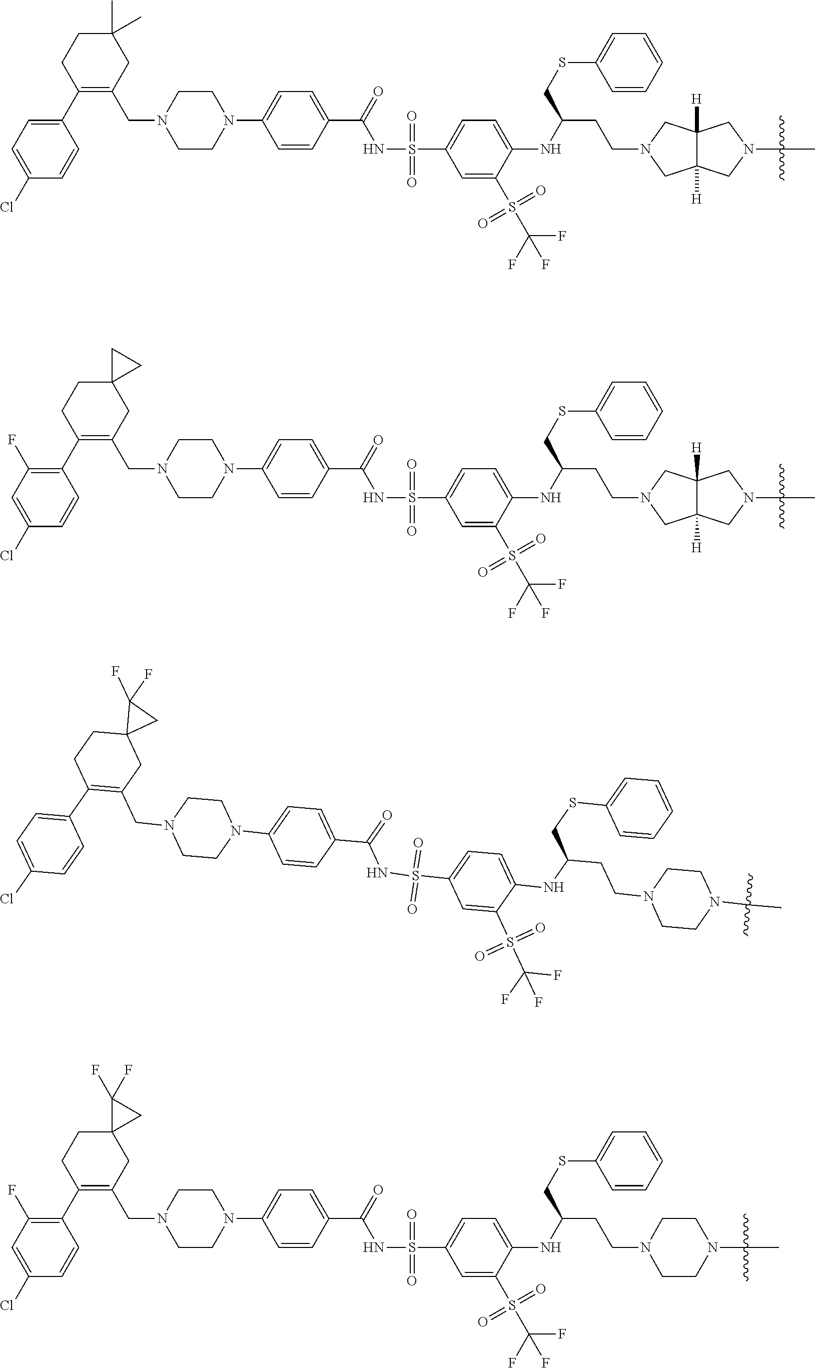

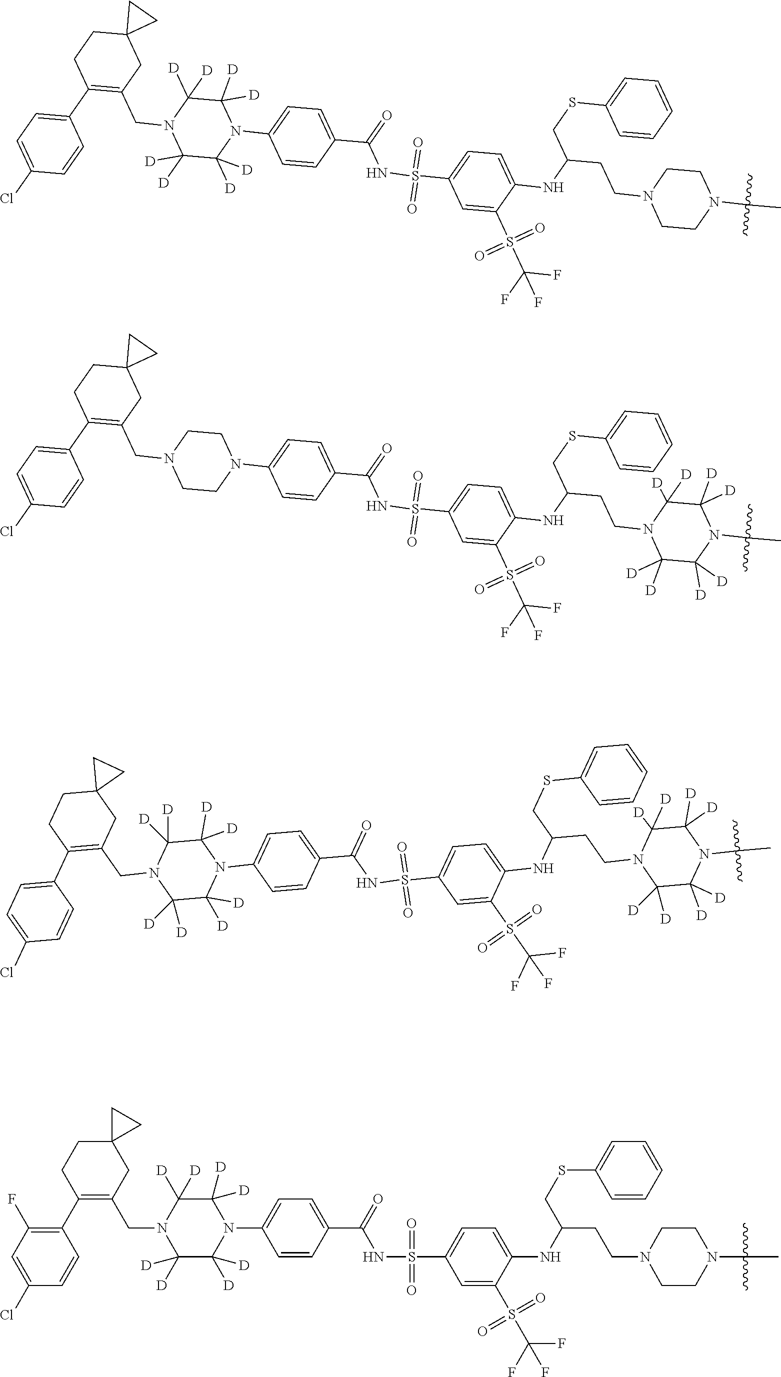

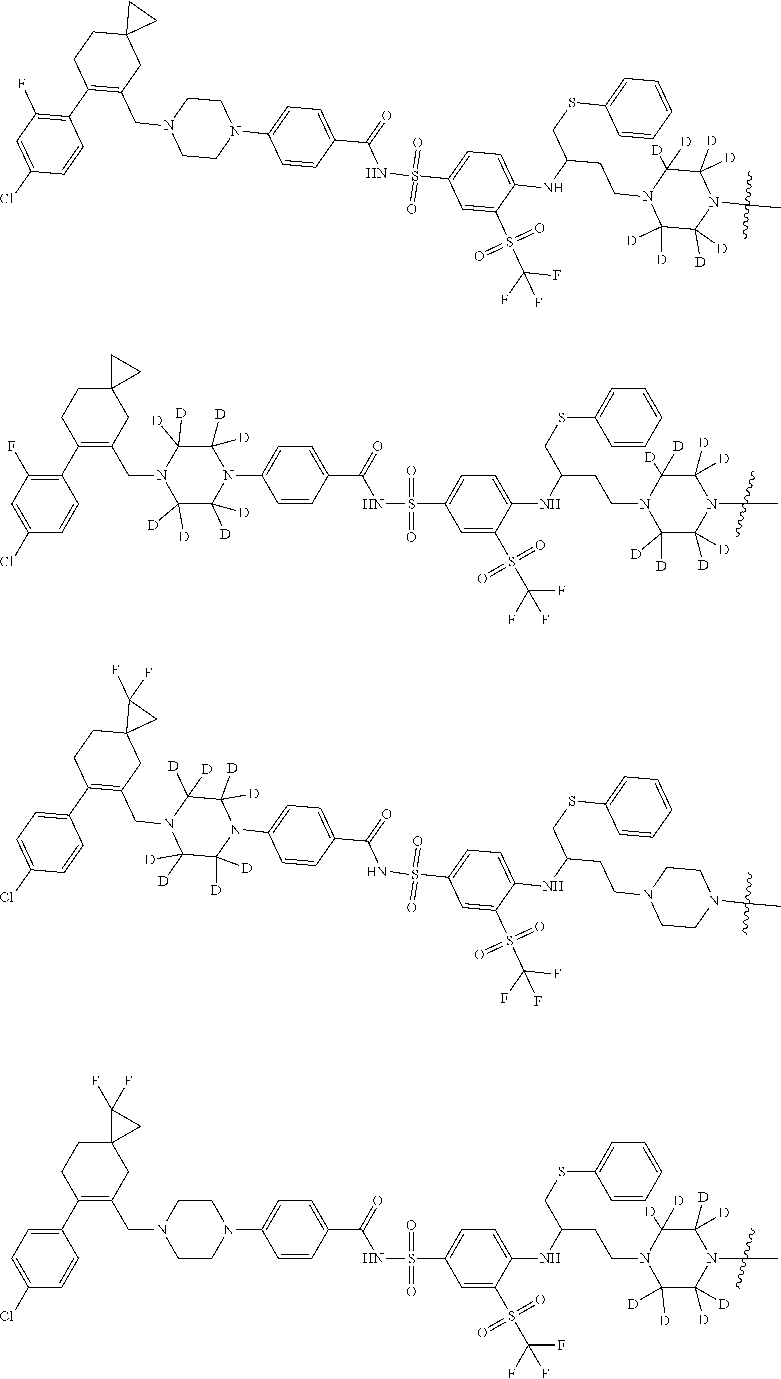

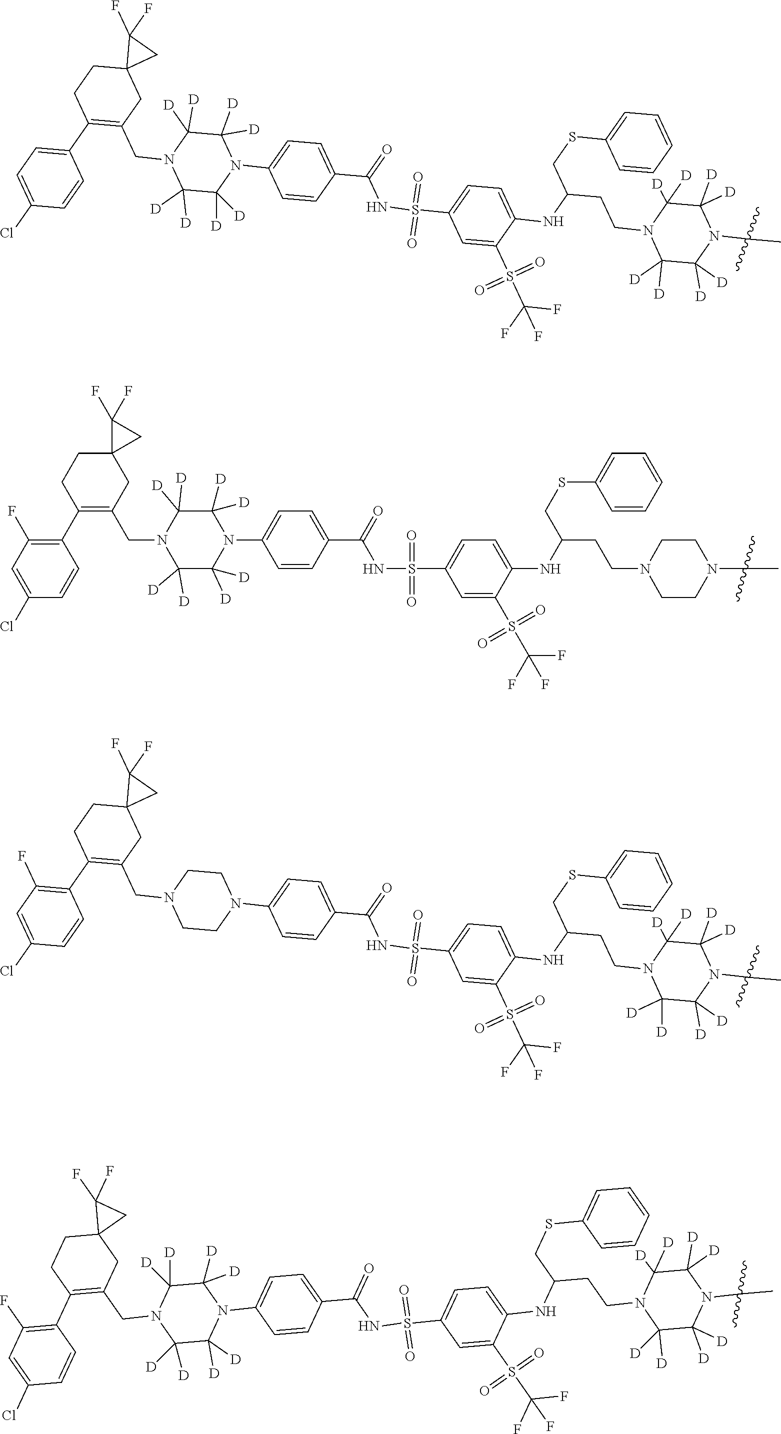

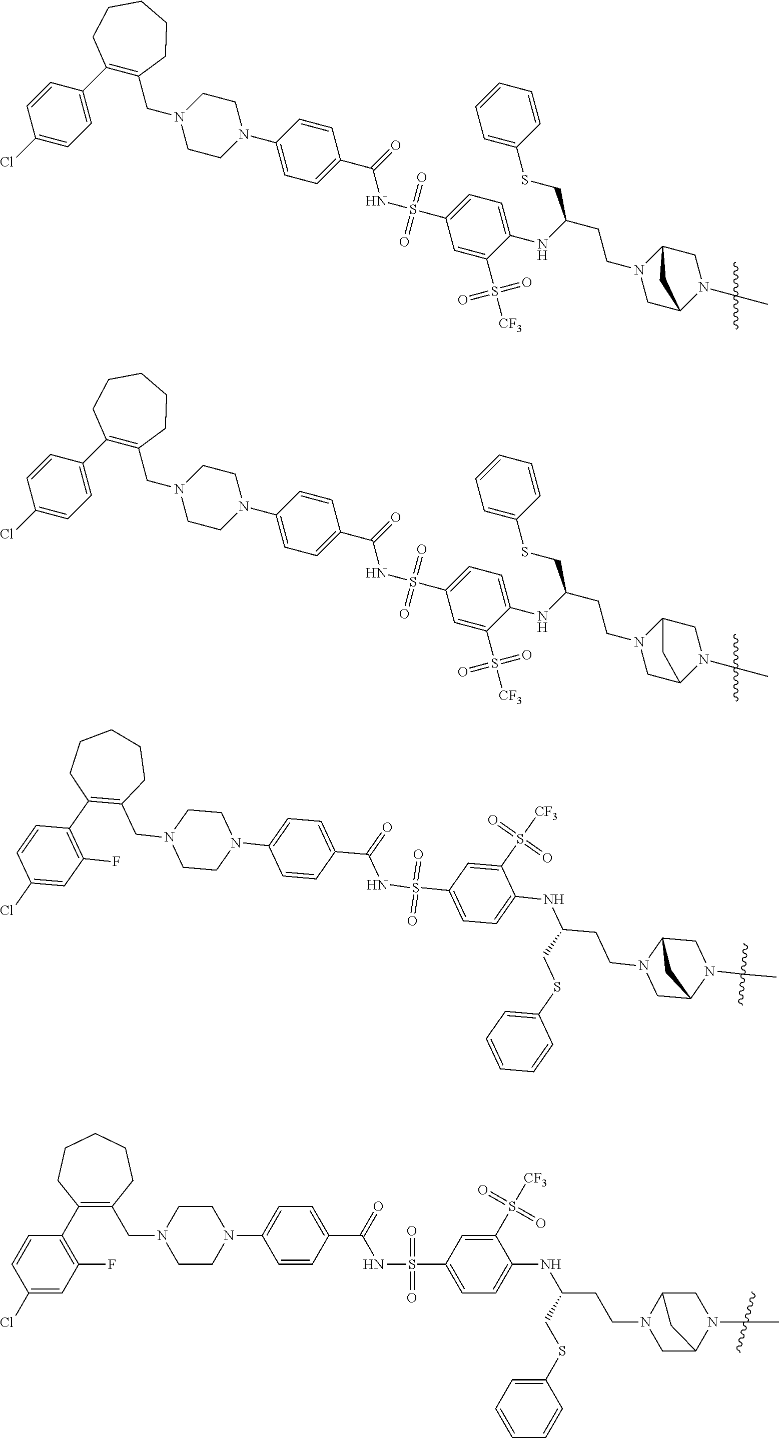

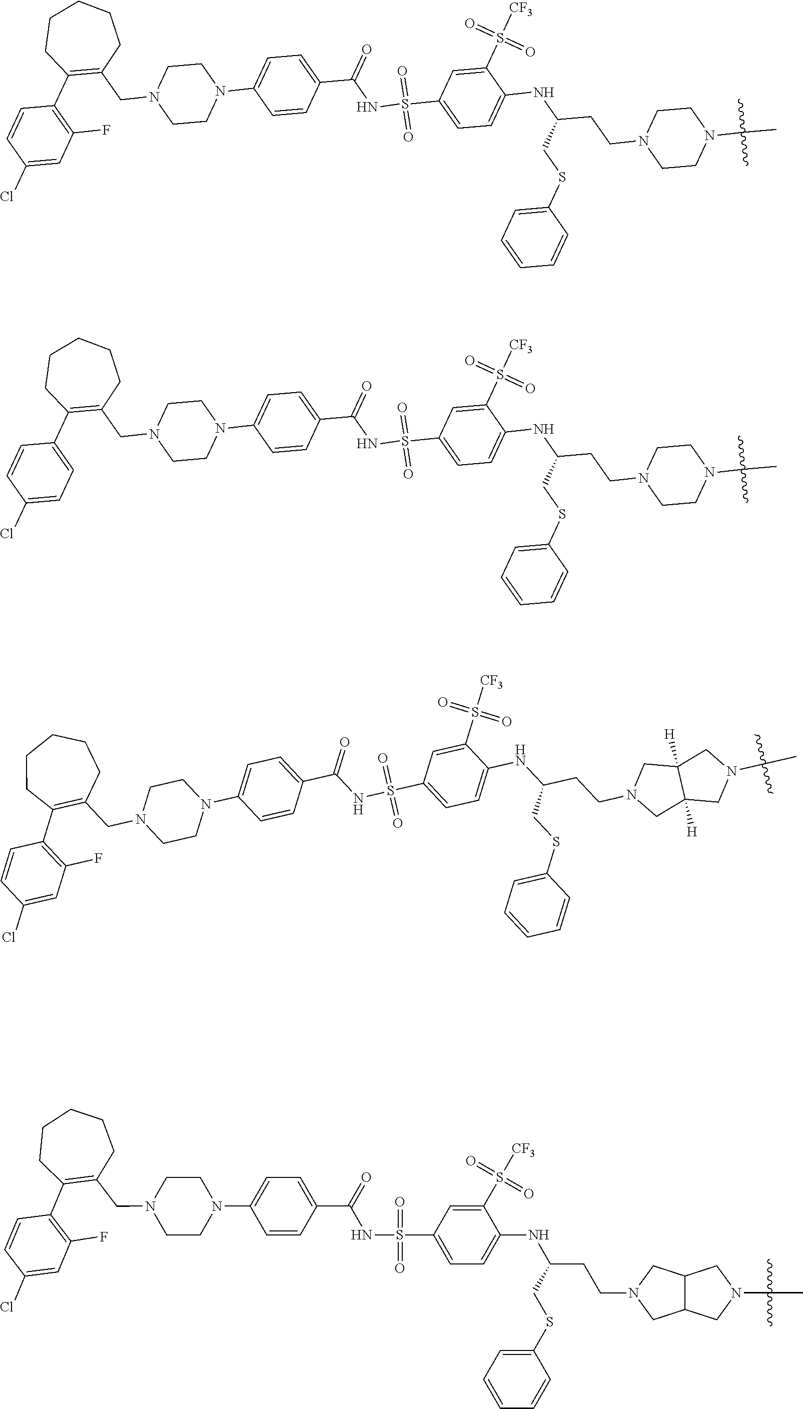

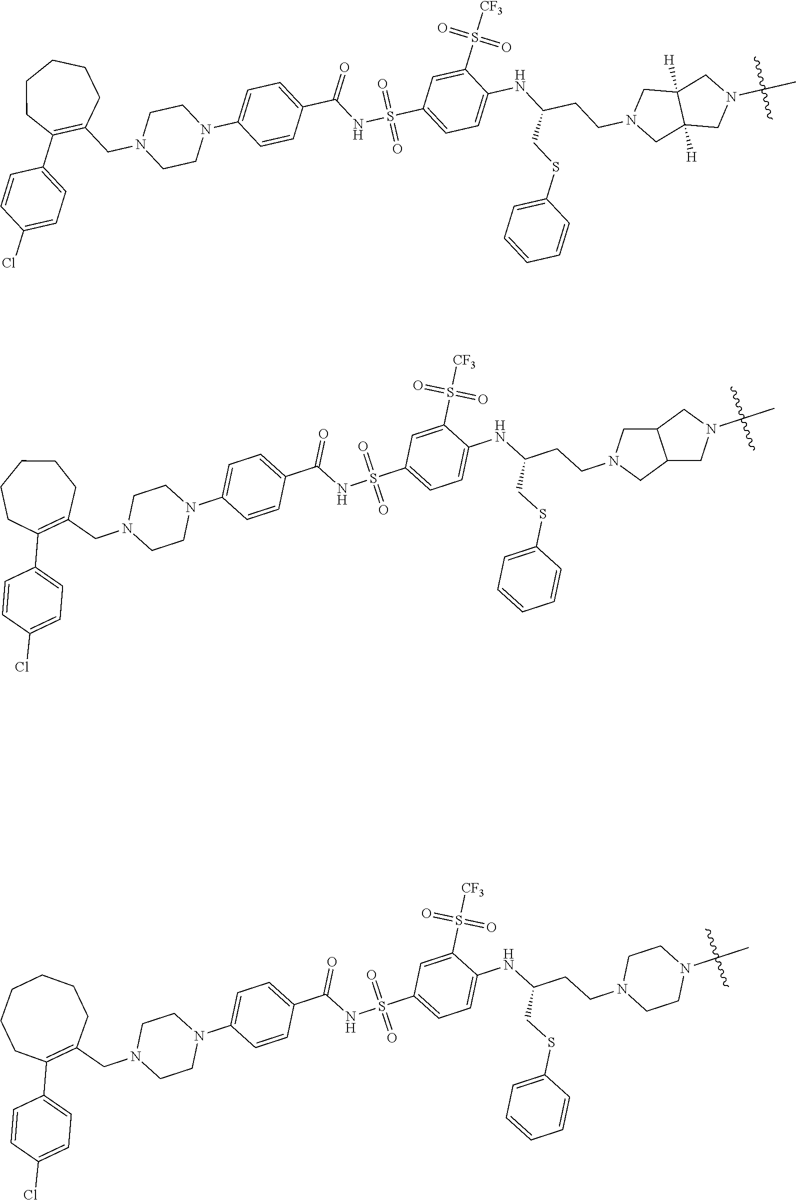 [SMILES: *N1C[C@@H]2CN(CC[C@H](CSc3ccccc3)Nc3ccc(S(=O)(=O)NC(=O)c4ccc(N5CCN(CC6=C(c7ccc(Cl)cc7)CCCCC6)CC5)cc4)cc3S(=O)(=O)C(F)(F)F)C[C@@H]2C1 ; *N1CC2CN(CC[C@H](CSc3ccccc3)Nc3ccc(S(=O)(=O)NC(=O)c4ccc(N5CCN(CC6=C(c7ccc(Cl)cc7)CCCCC6)CC5)cc4)cc3S(=O)(=O)C(F)(F)F)CC2C1 ; *N1CCN(CC[C@H](CSc2ccccc2)Nc2ccc(S(=O)(=O)NC(=O)c3ccc(N4CCN(C/C5=C(\c6ccc(Cl)cc6)CCCCCC5)CC4)cc3)cc2S(=O)(=O)C(F)(F)F)CC1]

K is selected from one of the following structural fragments (Table K-a):

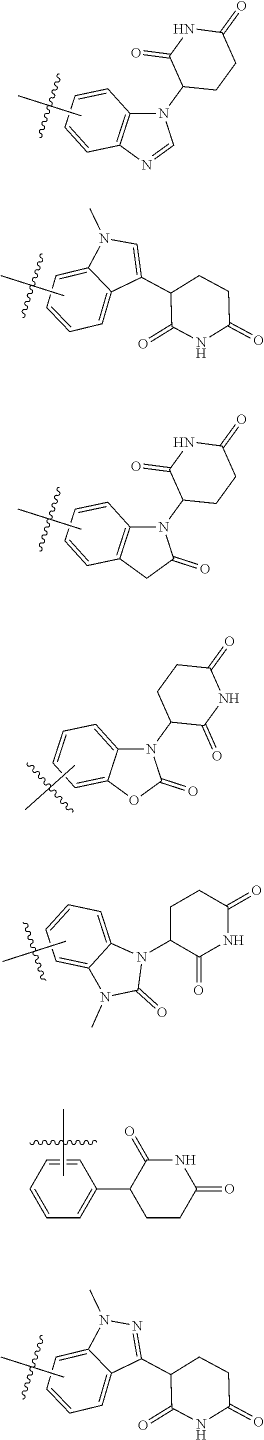

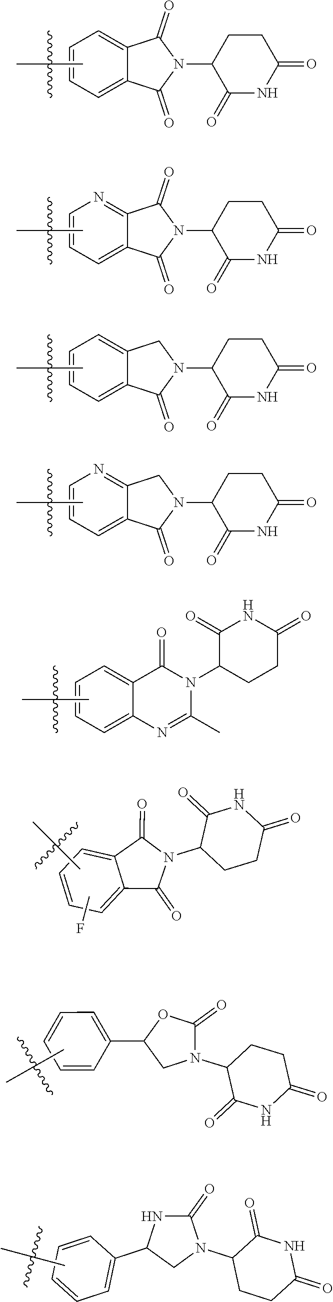

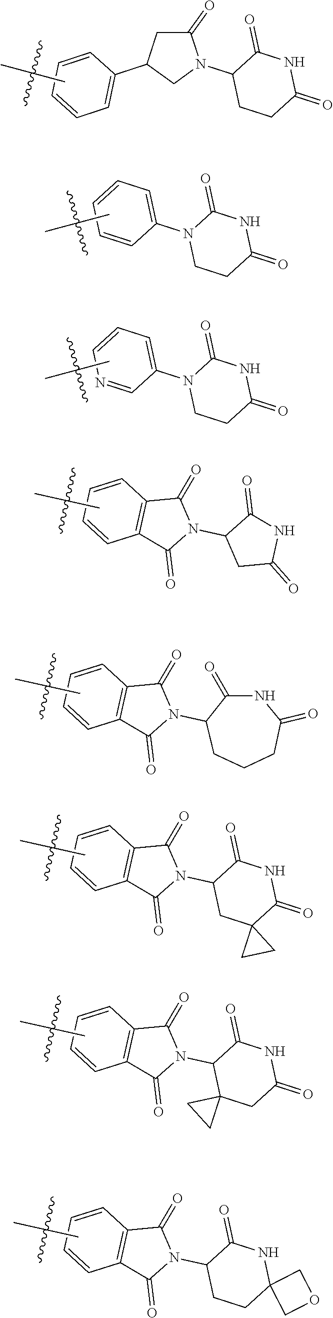

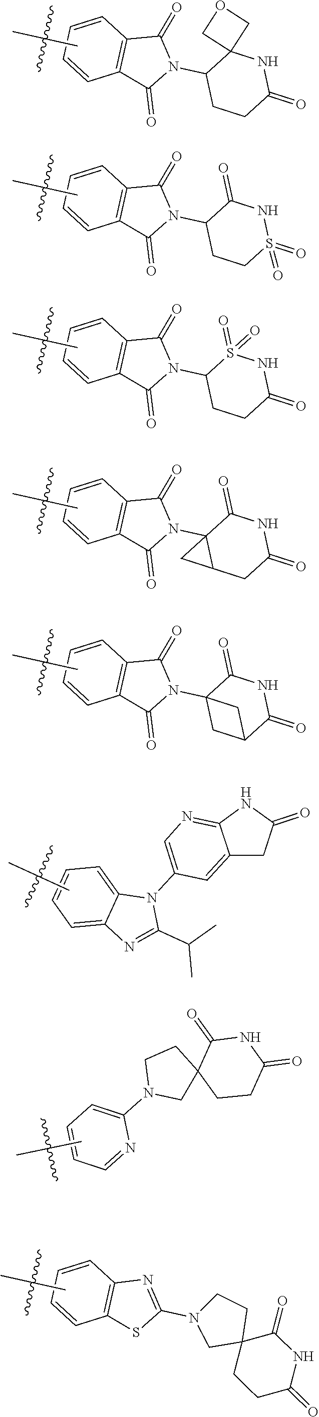

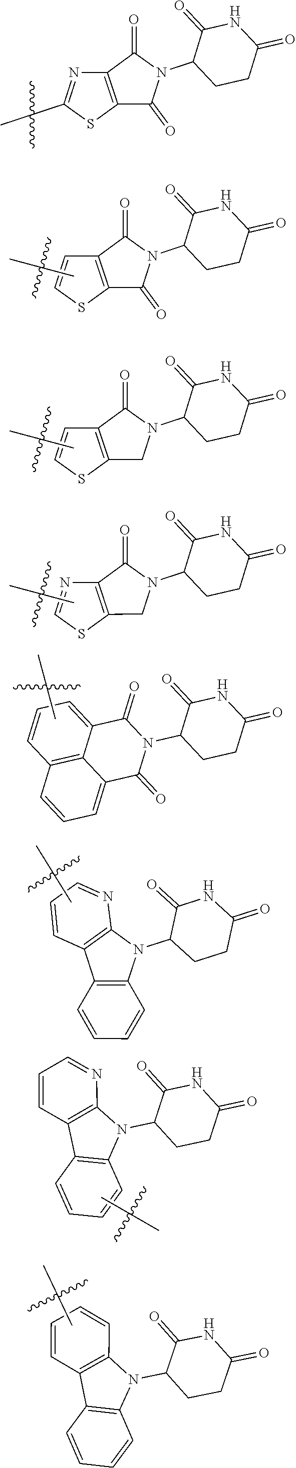

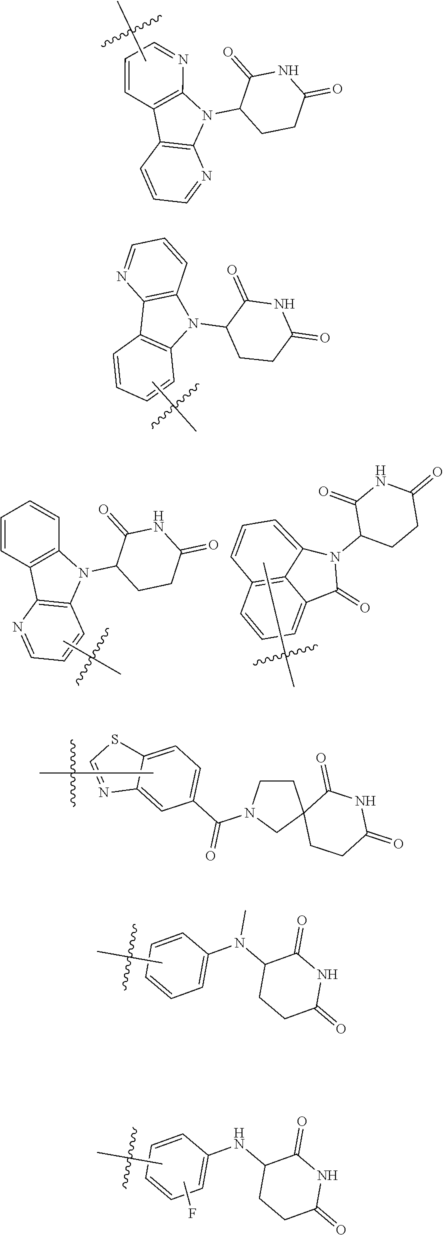

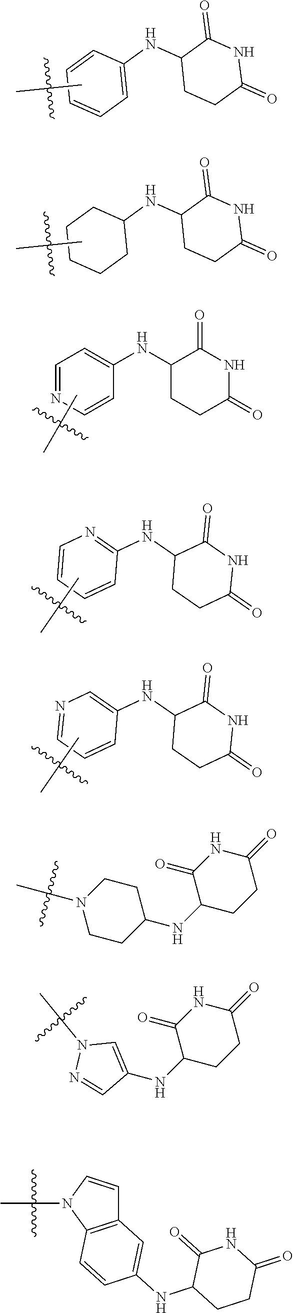

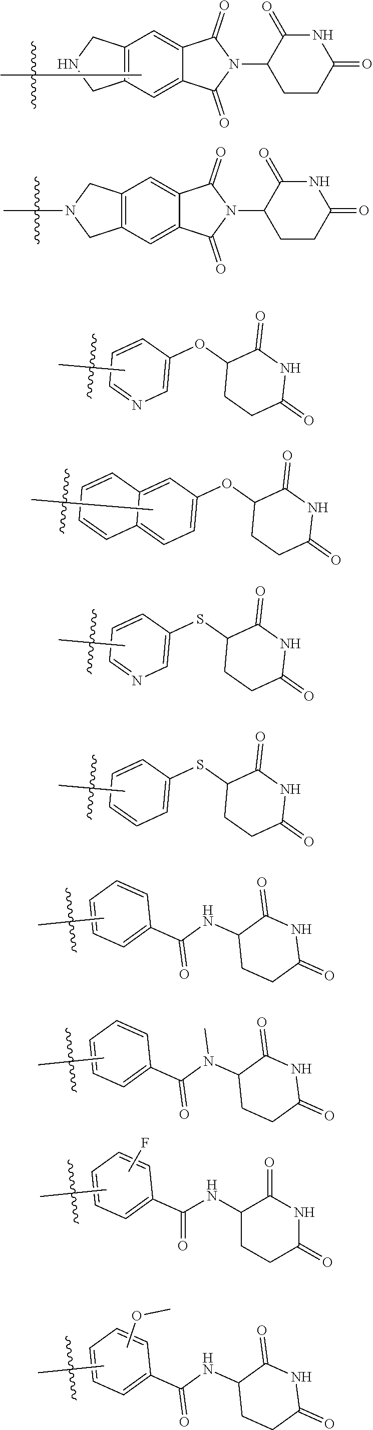

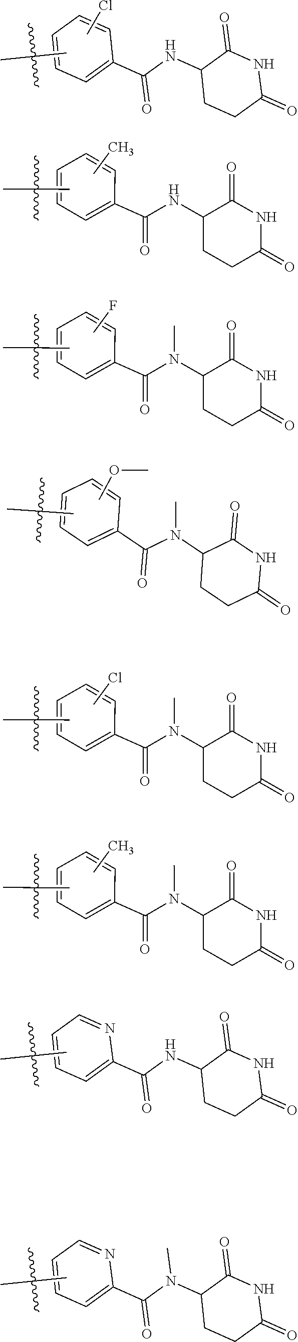

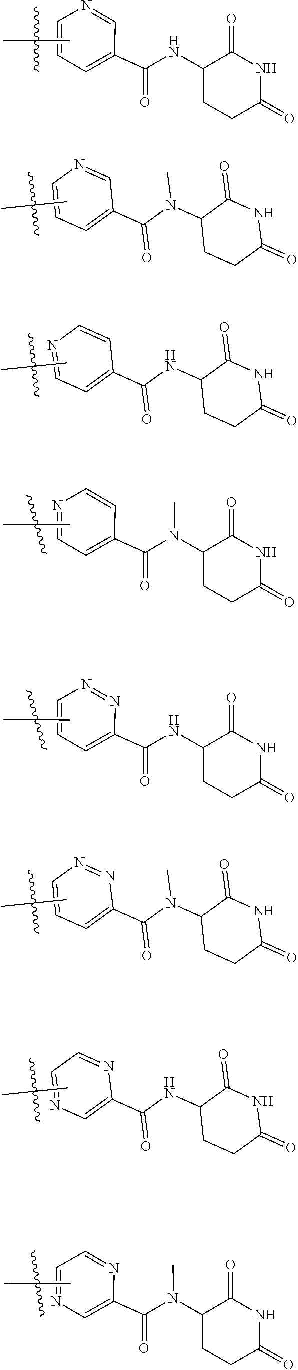

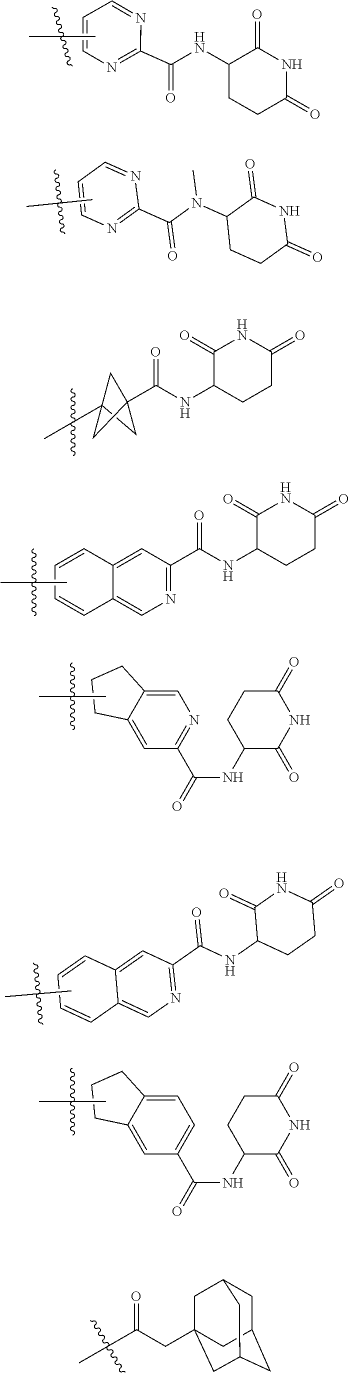

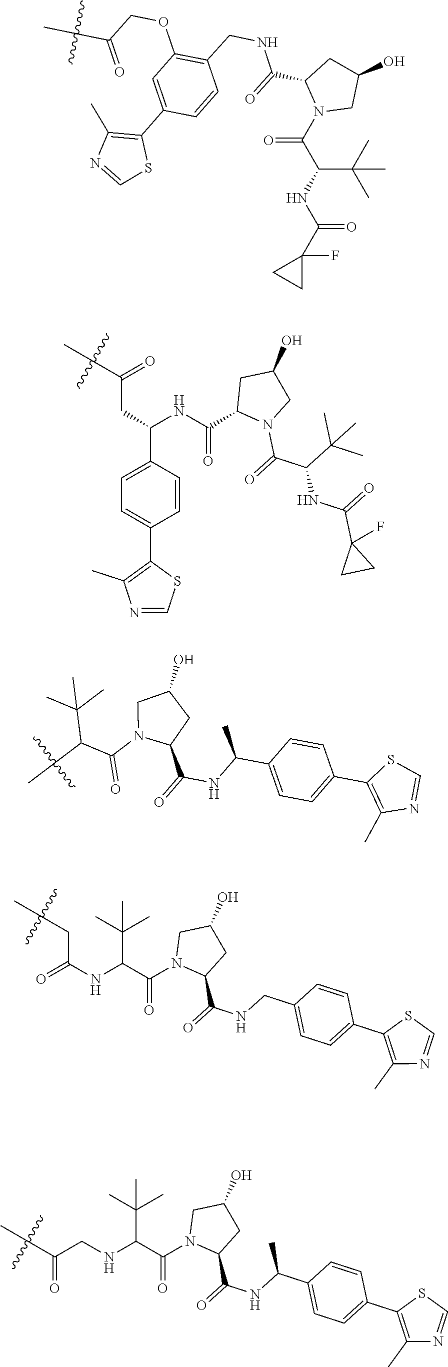 [SMILES: *C(=O)COc1cc(-c2scnc2C)ccc1CNC(=O)[C@@H]1C[C@@H](O)CN1C(=O)[C@@H](NC(=O)C1(F)CC1)C(C)(C)C ; *C(=O)C[C@H](NC(=O)[C@@H]1C[C@@H](O)CN1C(=O)[C@@H](NC(=O)C1(F)CC1)C(C)(C)C)c1ccc(-c2scnc2C)cc1 ; *C(C(=O)N1C[C@H](O)C[C@H]1C(=O)N[C@@H](C)c1ccc(-c2scnc2C)cc1)C(C)(C)C ; *CC(=O)NC(C(=O)N1C[C@H](O)C[C@H]1C(=O)NCc1ccc(-c2scnc2C)cc1)C(C)(C)C ; *C(=O)CNC(C(=O)N1C[C@H](O)C[C@H]1C(=O)N[C@@H](C)c1ccc(-c2scnc2C)cc1)C(C)(C)C]

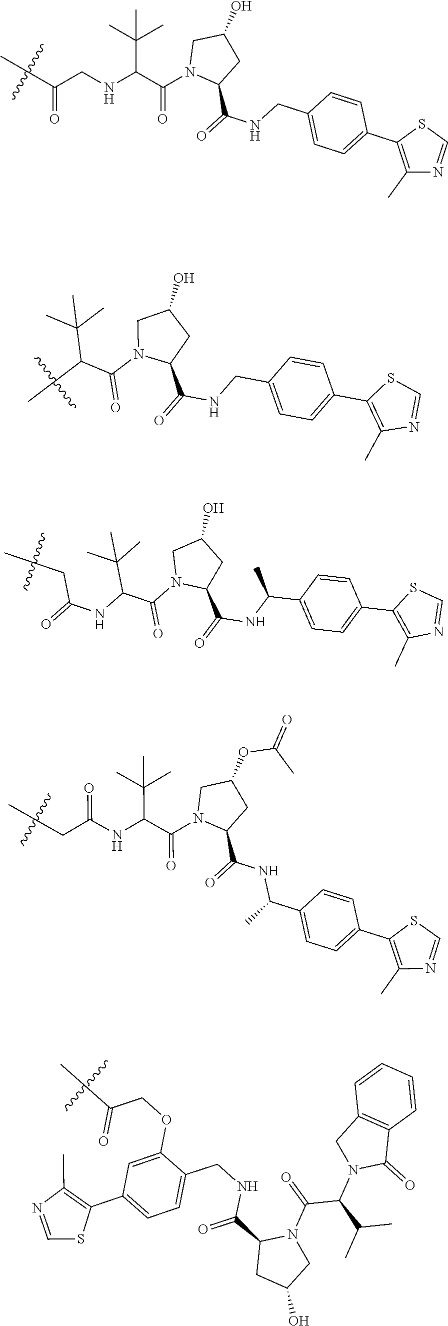 [SMILES: *C(=O)CNC(C(=O)N1C[C@H](O)C[C@H]1C(=O)NCc1ccc(-c2scnc2C)cc1)C(C)(C)C ; *C(C(=O)N1C[C@H](O)C[C@H]1C(=O)NCc1ccc(-c2scnc2C)cc1)C(C)(C)C ; *CC(=O)NC(C(=O)N1C[C@H](O)C[C@H]1C(=O)N[C@@H](C)c1ccc(-c2scnc2C)cc1)C(C)(C)C ; *CC(=O)NC(C(=O)N1C[C@H](OC(C)=O)C[C@H]1C(=O)N[C@@H](C)c1ccc(-c2scnc2C)cc1)C(C)(C)C ; *C(=O)COc1cc(-c2scnc2C)ccc1CNC(=O)[C@@H]1C[C@@H](O)CN1C(=O)[C@H](C(C)C)N1Cc2ccccc2C1=O]

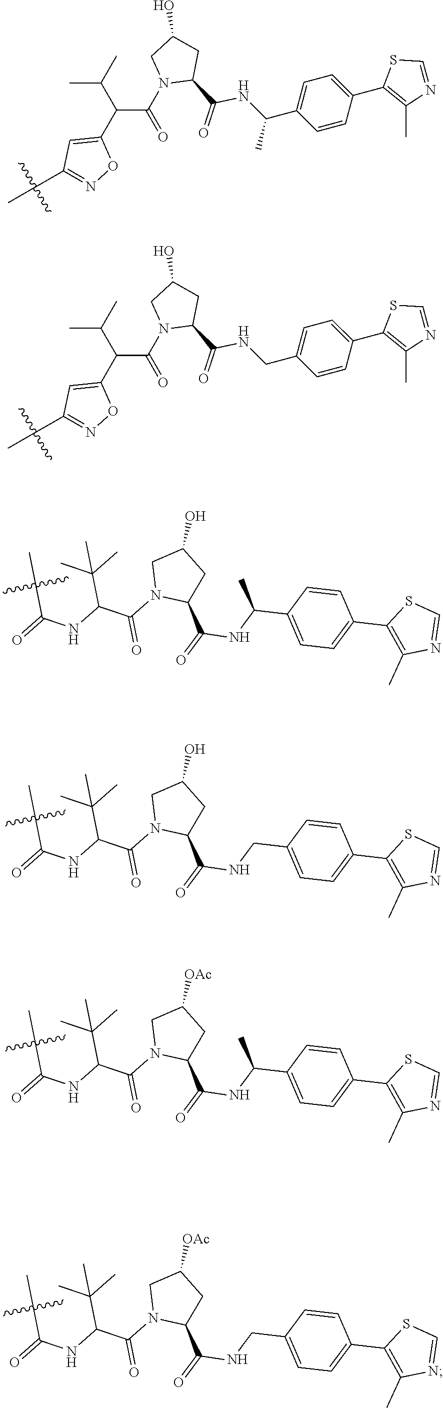 [SMILES: *c1cc(C(C(=O)N2C[C@H](O)C[C@H]2C(=O)N[C@@H](C)c2ccc(-c3scnc3C)cc2)C(C)C)on1 ; *c1cc(C(C(=O)N2C[C@H](O)C[C@H]2C(=O)NCc2ccc(-c3scnc3C)cc2)C(C)C)on1 ; *C(=O)NC(C(=O)N1C[C@H](O)C[C@H]1C(=O)N[C@@H](C)c1ccc(-c2scnc2C)cc1)C(C)(C)C ; *C(=O)NC(C(=O)N1C[C@H](O)C[C@H]1C(=O)NCc1ccc(-c2scnc2C)cc1)C(C)(C)C ; *C(=O)NC(C(=O)N1C[C@H](OC(C)=O)C[C@H]1C(=O)N[C@@H](C)c1ccc(-c2scnc2C)cc1)C(C)(C)C ; *C(=O)NC(C(=O)N1C[C@H](OC(C)=O)C[C@H]1C(=O)NCc1ccc(-c2scnc2C)cc1)C(C)(C)C]

the definition of other groups are the same as those in any one of the first, second, third and fourth embodiment of the present invention.

As a sixth embodiment of the present invention, provided is the above-mentioned compound of general formula (I) or the stereoisomer, deuterated compound, solvate, prodrug metabolite, pharmaceutically acceptable salt or co-crystal thereof, wherein
L is selected from a bond, -Ak1-, -Ak1-Ak2-, -Ak1-Ak2-Ak3-, -Ak1-Ak2-Ak3-Ak4-, -Ak1-Ak2-Ak3-Ak4-Ak5-, -Ak1-Ak2-Ak3-Ak4-Ak5-Ak6-, -Cy1-, -Cy1-Ak1-, -Cy1-Ak1-Ak2-, -Cy1-Ak1-Ak2-Ak3-, -Cy1-Ak1-Ak2-Ak3-Ak4-, -Cy1-Cy2-, -Cy1-Ak1-Cy2-, -Cy1-Cy2-Ak2-, -Cy1-Ak1-Cy2-Ak2-, -Cy1-Ak1-Cy2-Ak2-Ak3-, -Cy1-Ak1-Cy2-Ak2-Ak3-Ak4-, -Cy1-Cy2-Ak2-Ak3-, -Cy1-Cy2-Ak2-Ak3-Ak4-, -Cy1-Ak1-Ak2-Cy3-, -Cy1-Ak1-Ak2-Cy3-Ak3-, -Cy1-Cy2-Cy3-, -Cy1-Ak1-Cy2-Cy3-, -Cy1-Cy2-Ak2-Cy3-, -Cy1-Cy2-Cy3-Ak3-, -Cy1-Ak1-Cy2-Cy3-Ak3-, -Cy1-Cy2-Ak2-Cy3-Ak3-, -Cy1-Ak1-Cy2-Ak2-Cy3-, -Cy1-Ak1-Cy2-Ak2-Cy3-Ak3-, -Cy1-Cy2-Cy3-Ak3-Ak4-, -Cy1-Cy2-Cy3-Ak3-Cy4-, -Cy1-Cy2-Cy3-Cy4-, -Cy1-Ak1-Cy2-Cy3-Cy4-, -Cy1-Cy2-Ak2-Cy3-Cy4-, -Cy1-Cy2-Cy3-Ak3-Cy4-, -Cy1-Cy2-Cy3-Cy4-Ak4-, -Cy1-Ak1-Cy2-Ak2-Cy3-Ak3-Cy4-, -Cy1-Ak1-Cy2-Ak2-Cy3-Cy4-, -Ak1-Cy2-, -Ak1-Cy2-Cy3-, -Ak1-Ak2-Cy3-, -Ak1-Ak2-Cy3-Cy4-, -Ak1-Cy2-Ak2-Cy3-, -Ak1-Cy2-Cy3-Ak3-Cy4-, -Ak1-Cy2-Cy3-Cy4-Ak4-Cy5-, -Ak1-Cy2-Ak2-, -Cy1-Cy2-Cy3-Ak3-Ak4-Ak5-, -Cy1-Cy2-Ak2-Cy3-Ak3-Ak4-Ak5-, -Cy1-Ak1-Cy2-Ak2-Ak3-Ak4-Ak5-, -Cy1-Cy2-Cy3-Cy4-Ak4-Ak5-, -Cy1-Ak1-Ak2-Ak3-Ak4-Ak5-, -Ak1-Cy2-Ak2-Ak3-Ak4-Ak5-, -Ak1-Cy2-Ak2-Ak3-Ak4-, -Ak1-Cy2-Ak2-Ak3-, -Ak1-Ak2-Ak3-Ak4-Ak5-, -Ak1-Ak2-Ak3-Ak4-Ak5-Ak6-, or -Ak1-Ak2-Ak3-Ak4-Ak5-Ak6-Ak7-;
the definitions of other groups are the same as those in any one of the first, second, third, fourth and fifth embodiments of the present invention.

As a seventh embodiment of the present invention, provided is the above-mentioned compound of general formula (I) or the stereoisomer, deuterated compound, solvate, prodrug, metabolite, pharmaceutically acceptable salt or co-crystal thereof, wherein
L is selected from a bond or a group shown in Table B-1, wherein the left side of the group is linked to B;
the definitions of other groups are the same as those in any one of the first, second, third, fourth, fifth and sixth embodiments of the present invention.

As an eighth embodiment of the present invention, provided is the above-mentioned compound of general formula (I) or the stereoisomer, deuterated compound, solvate, prodrug, metabolite, pharmaceutically acceptable salt or co-crystal thereof, wherein K is selected from one of the following structural fragments:

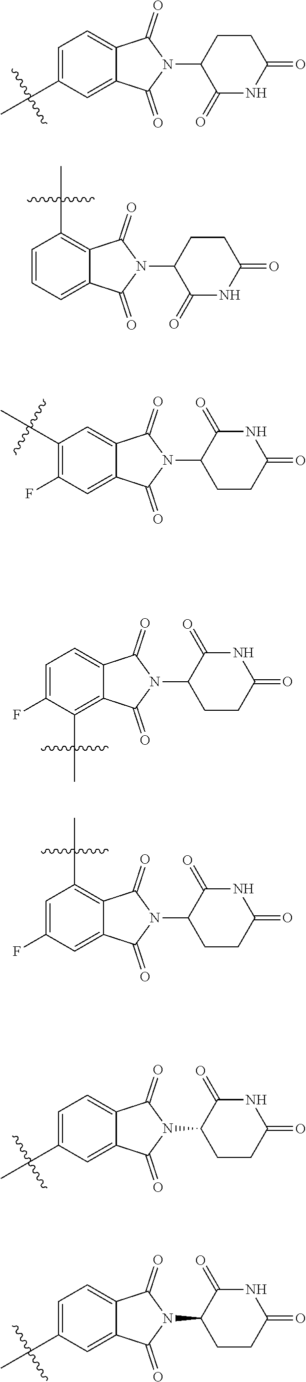

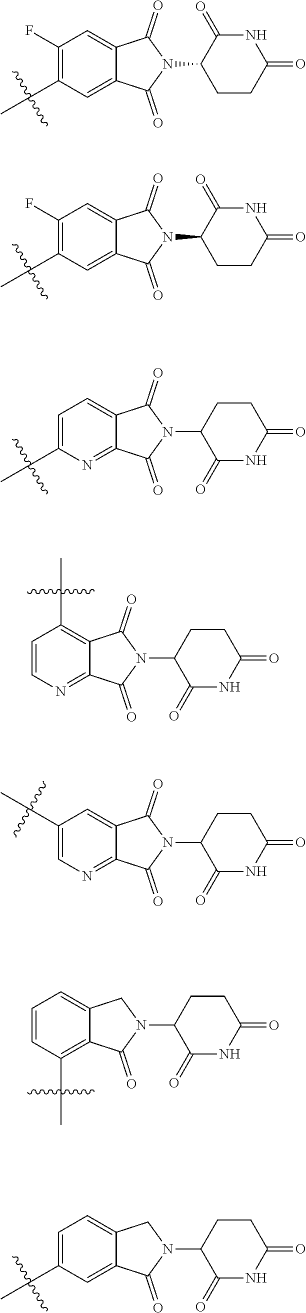

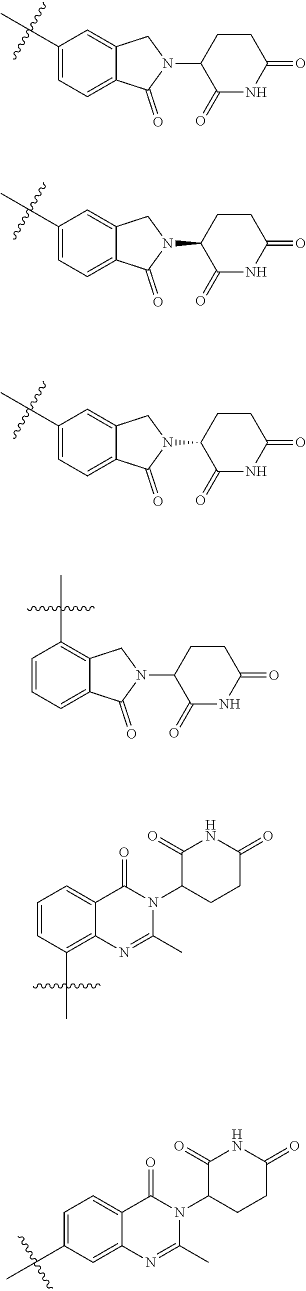

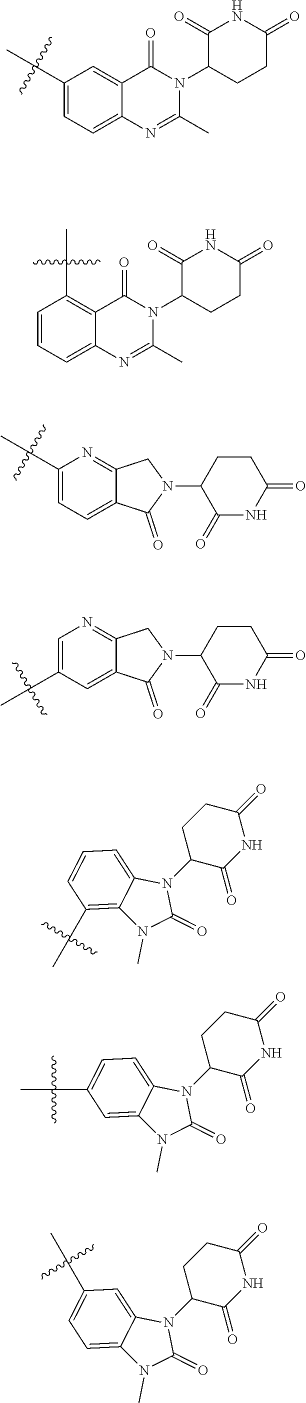

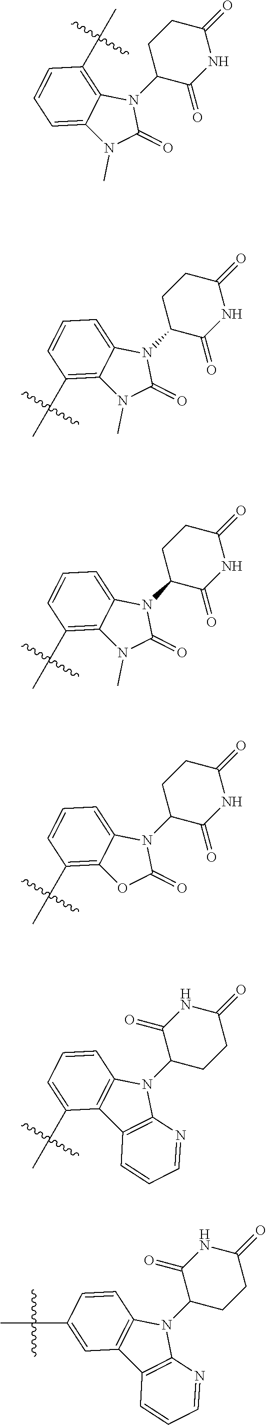

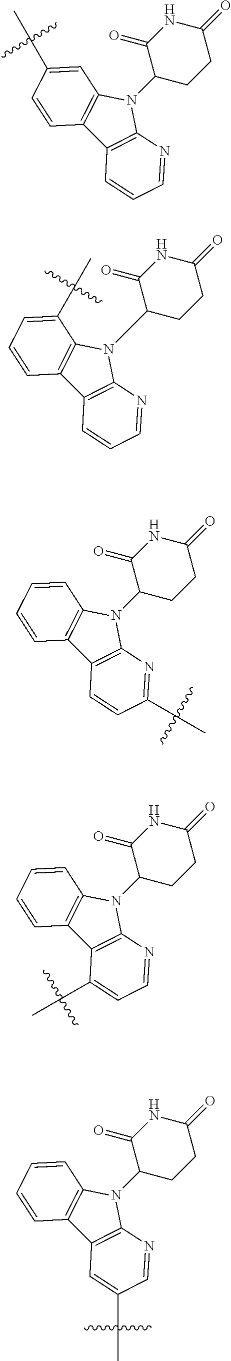

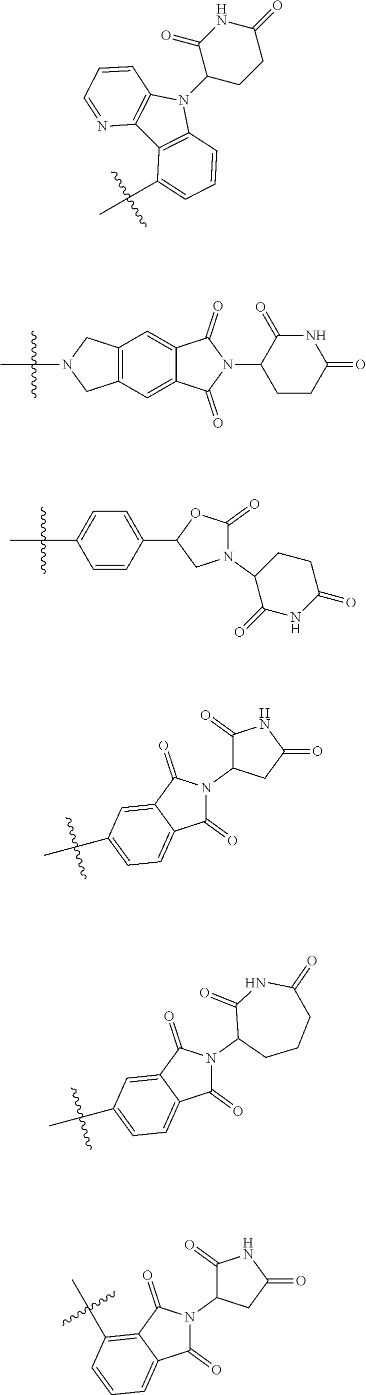

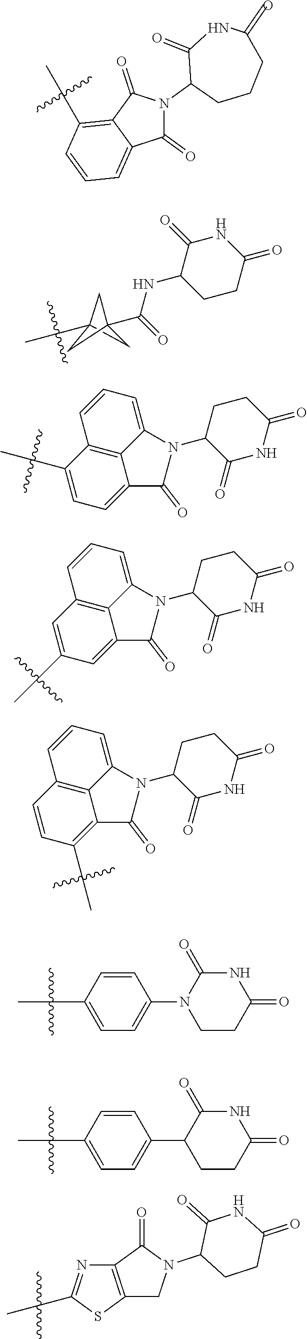

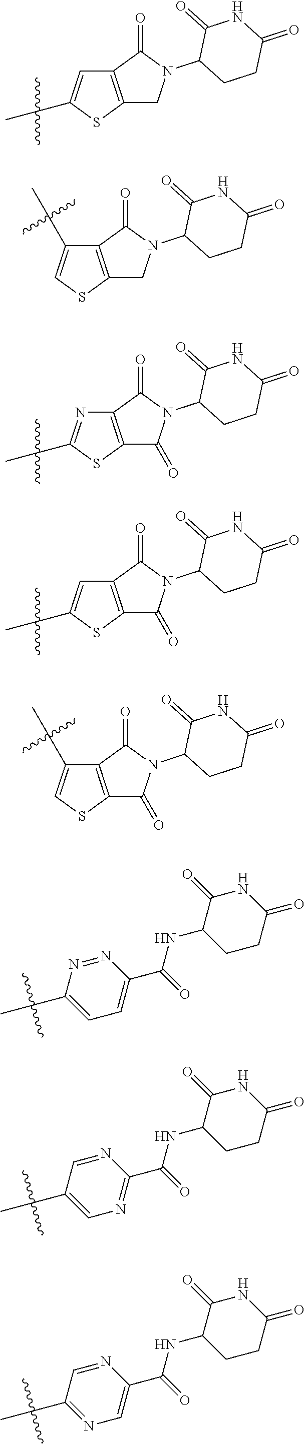

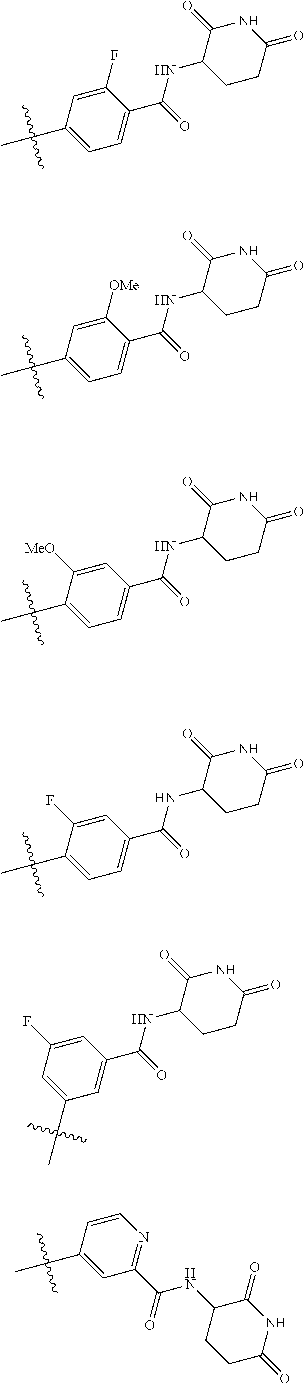

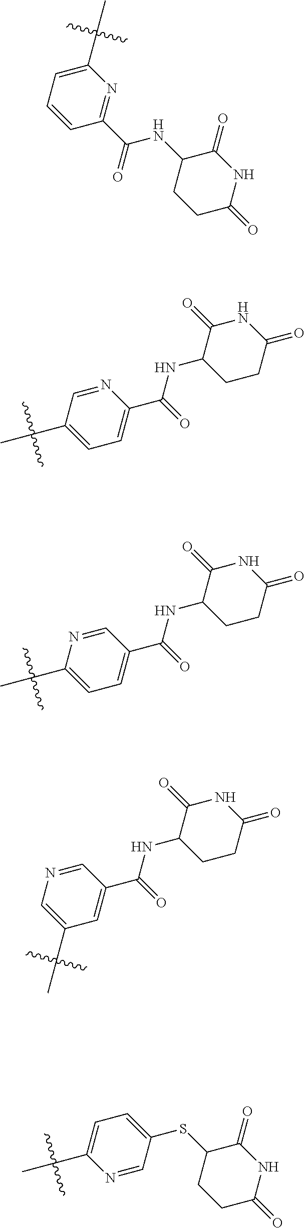

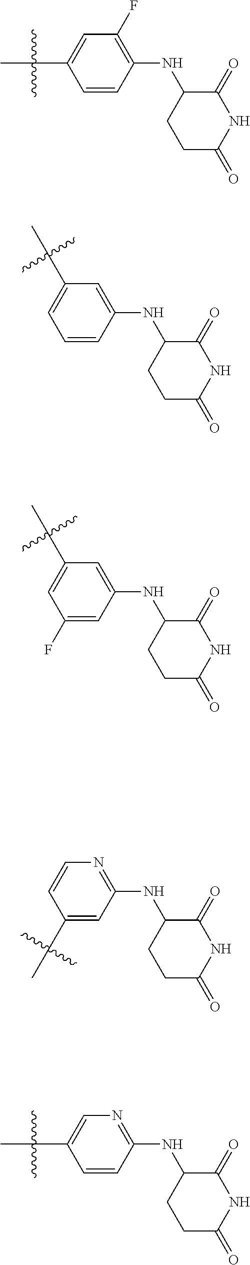

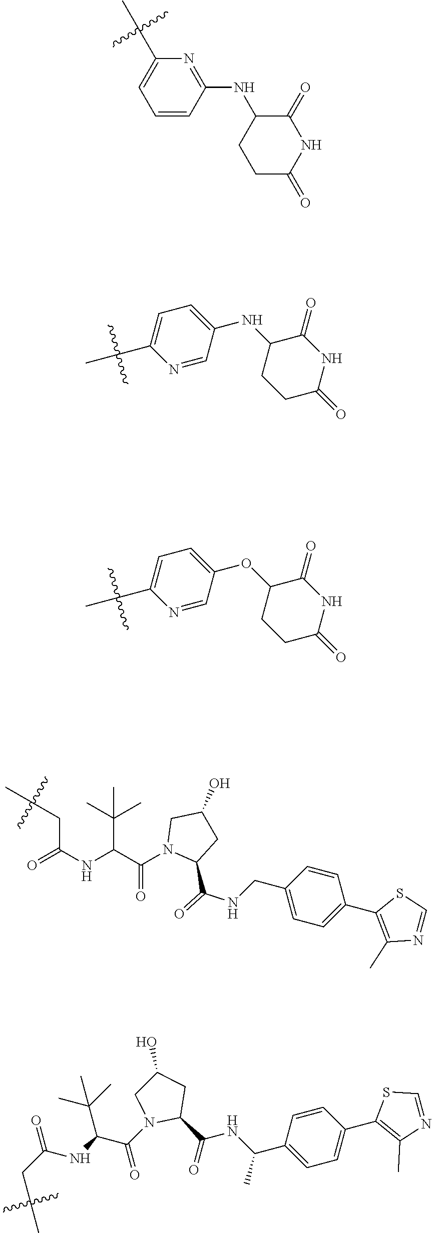

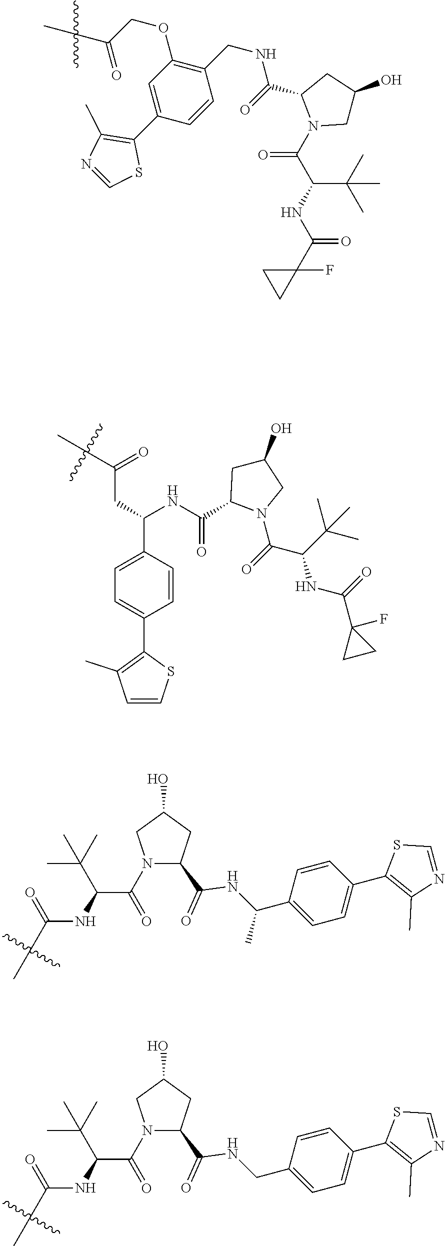

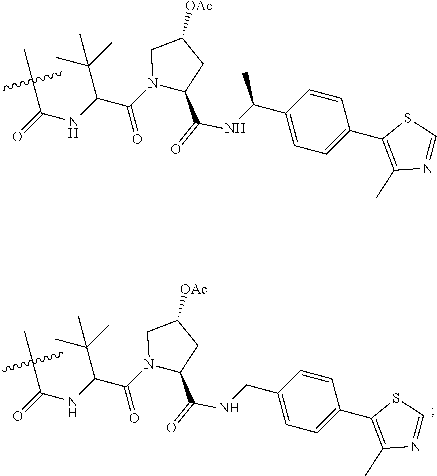

the definitions of other groups are the same as those in any one of the first, second third, fourth, fifth, sixth and seventh embodiments of the present invention.

As a ninth embodiment of the present invention, provided is the above-mentioned compound of general formula (I) or the stereoisomer, deuterated compound, solvate, prodrug, metabolite, pharmaceutically acceptable salt or co-crystal thereof, wherein the compound of general formula (I) is selected from the compound of general formula (Ia)

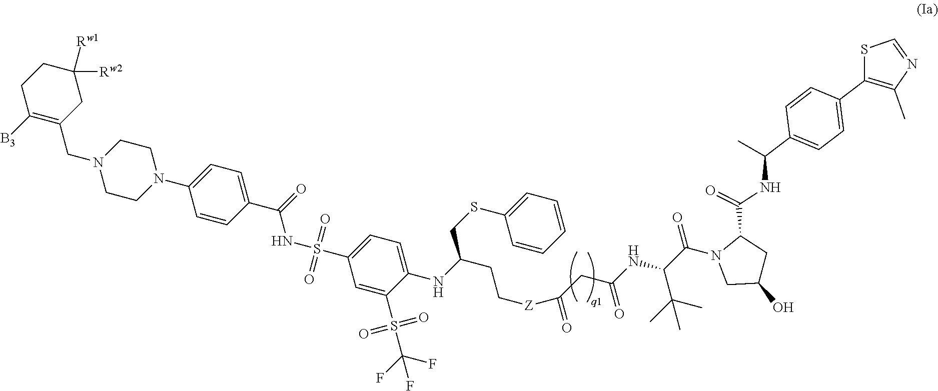

q1 is selected from 1, 2, 3, 4, 5, 6, 7 or 8;

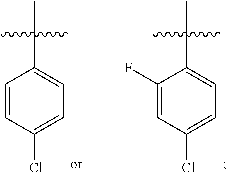 [SMILES: *c1ccc(Cl)cc1 ; *c1ccc(Cl)cc1F]

B₃ is selected from or
R^{w1} and R^{w2} are selected from methyl;
or R^{w1} and R^{w2} are directly connected to form, together with the carbon atom to which they are attached, cyclopropyl;
Z is selected from

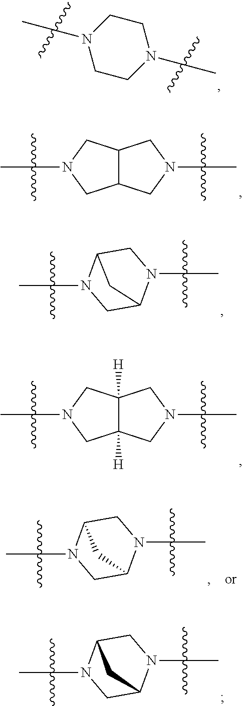 [SMILES: *N1CCN(*)CC1 ; *N1CC2CN(*)CC2C1 ; *N1CC2CC1CN2* ; *N1C[C@H]2CN(*)C[C@H]2C1 ; *N1C[C@H]2C[C@@H]1CN2* ; *N1C[C@@H]2C[C@H]1CN2*]

when Z is selected from

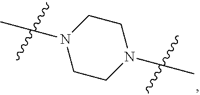 [SMILES: *N1CCN(*)CC1]

and R^{w1} and R^{w2} are selected from methyl, B₃ is selected from

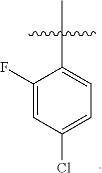 [SMILES: *c1ccc(Cl)cc1F]

As a tenth embodiment of the present invention, provided is the above-mentioned compound of general formula (I) or the stereoisomer, deuterated compound, solvate, prodrug, metabolite, pharmaceutically acceptable salt or co-crystal thereof, wherein the compound of general formula (I) is selected from the compound of general formula (Ib),

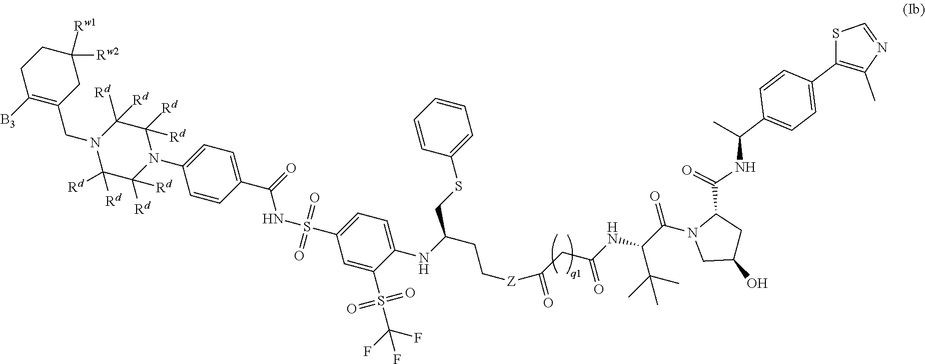

q1 is selected from 1, 2, 3, 4, 5, 6, 7 or 8;
B₃ is selected from or;

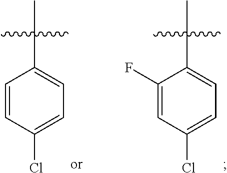 [SMILES: *c1ccc(Cl)cc1 ; *c1ccc(Cl)cc1F]

R^{w1} and R^{w2} are selected from methyl;
or R^{w1} and R^{w2} are directly connected to form, together with the carbon atom to which they are attached, cyclopropyl;
Z is selected from

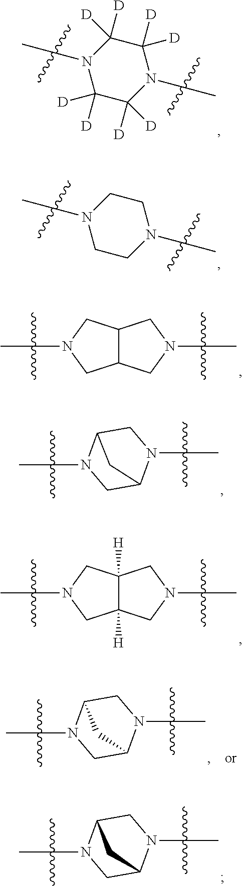

each R^{d} is independently selected from H or deuterium;
the compound of general formula (Ib) has at least one deuterium.

The present invention relates to a compound as described below or a stereoisomer, deuterated compound, solvate, prodrug, metabolite, pharmaceutically acceptable salt, or co-crystal thereof, wherein the compound is selected from one of the structures shown in Table P-1:

TABLE P-1

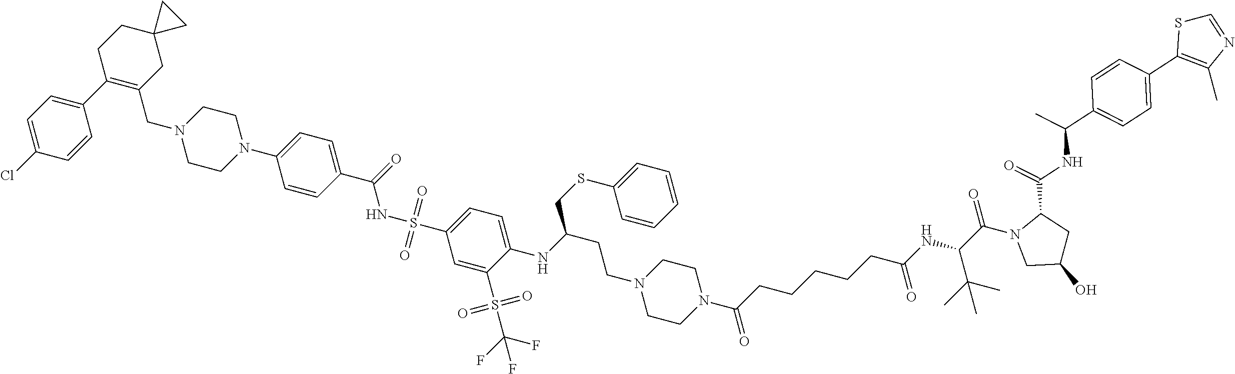 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC7(CC7)C6)CC5)cc4)cc3S(=O)(=O)C(F)(F)F)CC2)C(C)(C)C)cc1]

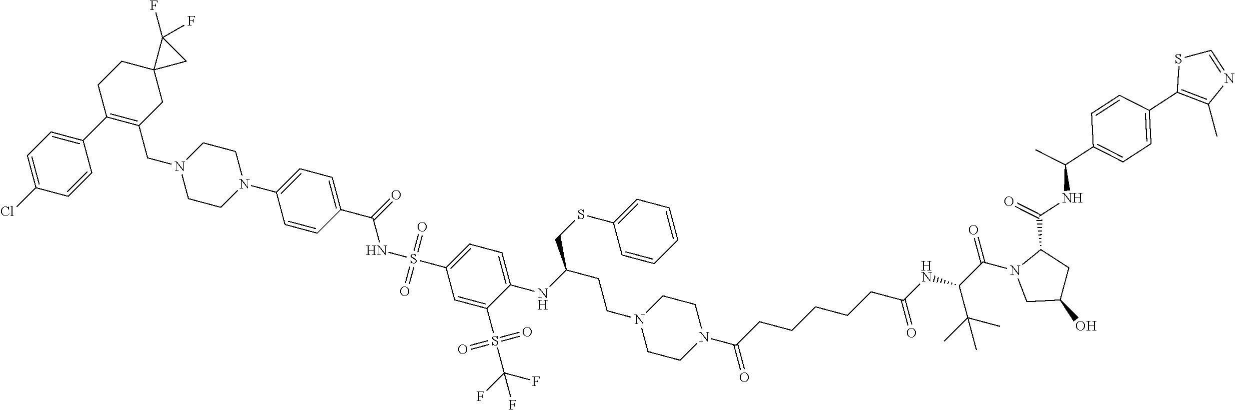 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC7(C6)CC7(F)F)CC5)cc4)cc3S(=O)(=O)C(F)(F)F)CC2)C(C)(C)C)cc1]

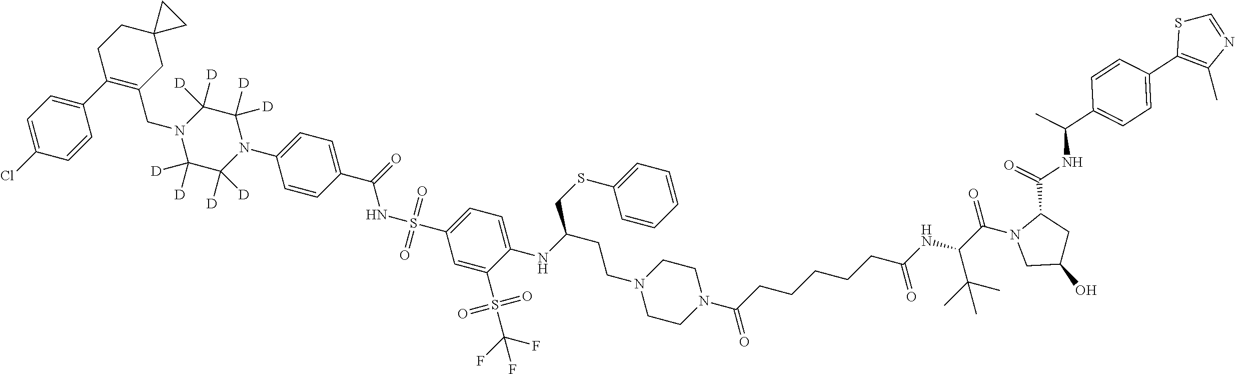 [SMILES: [2H]C1([2H])N(CC2=C(c3ccc(Cl)cc3)CCC3(CC3)C2)C([2H])([2H])C([2H])([2H])N(c2ccc(C(=O)NS(=O)(=O)c3ccc(N[C@H](CCN4CCN(C(=O)CCCCCC(=O)N[C@H](C(=O)N5C[C@H](O)C[C@H]5C(=O)N[C@@H](C)c5ccc(-c6scnc6C)cc5)C(C)(C)C)CC4)CSc4ccccc4)c(S(=O)(=O)C(F)(F)F)c3)cc2)C1([2H])[2H]]

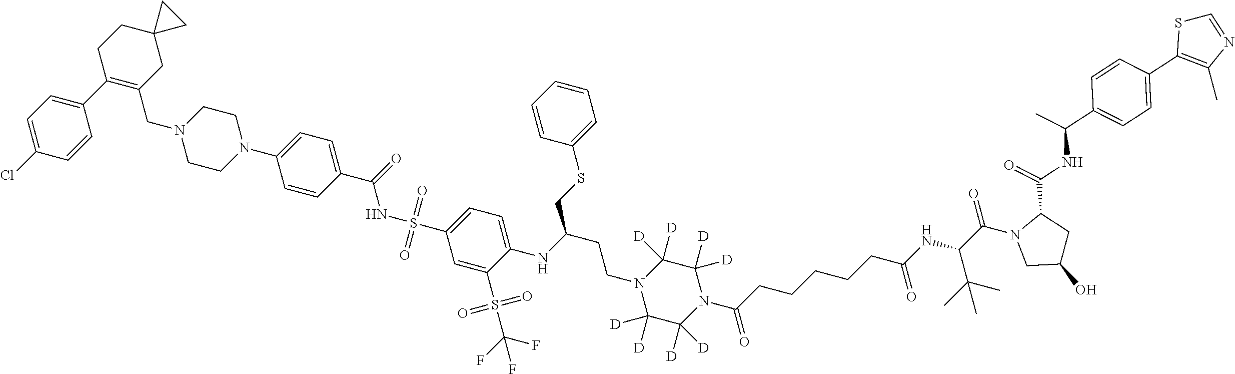 [SMILES: [2H]C1([2H])N(CC[C@H](CSc2ccccc2)Nc2ccc(S(=O)(=O)NC(=O)c3ccc(N4CCN(CC5=C(c6ccc(Cl)cc6)CCC6(CC6)C5)CC4)cc3)cc2S(=O)(=O)C(F)(F)F)C([2H])([2H])C([2H])([2H])N(C(=O)CCCCCC(=O)N[C@H](C(=O)N2C[C@H](O)C[C@H]2C(=O)N[C@@H](C)c2ccc(-c3scnc3C)cc2)C(C)(C)C)C1([2H])[2H]]

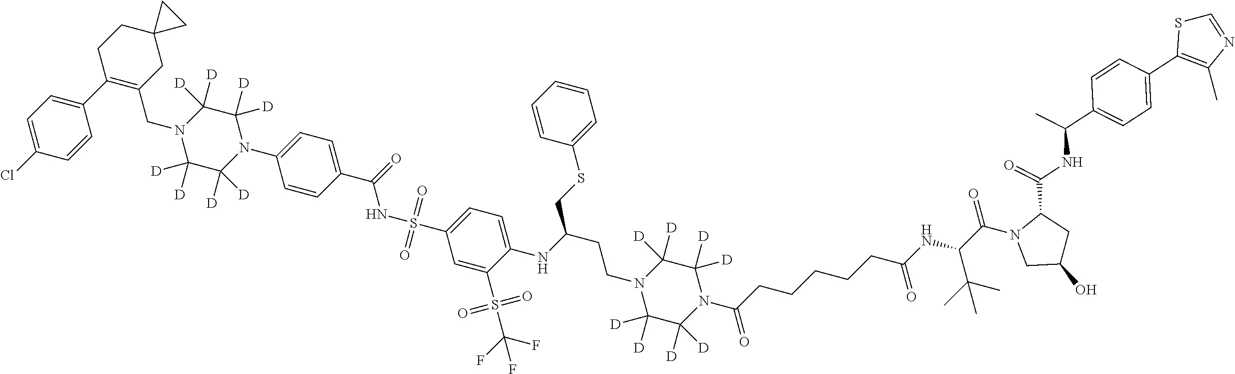 [SMILES: [2H]C1([2H])N(CC[C@H](CSc2ccccc2)Nc2ccc(S(=O)(=O)NC(=O)c3ccc(N4C([2H])([2H])C([2H])([2H])N(CC5=C(c6ccc(Cl)cc6)CCC6(CC6)C5)C([2H])([2H])C4([2H])[2H])cc3)cc2S(=O)(=O)C(F)(F)F)C([2H])([2H])C([2H])([2H])N(C(=O)CCCCCC(=O)N[C@H](C(=O)N2C[C@H](O)C[C@H]2C(=O)N[C@@H](C)c2ccc(-c3scnc3C)cc2)C(C)(C)C)C1([2H])[2H]]

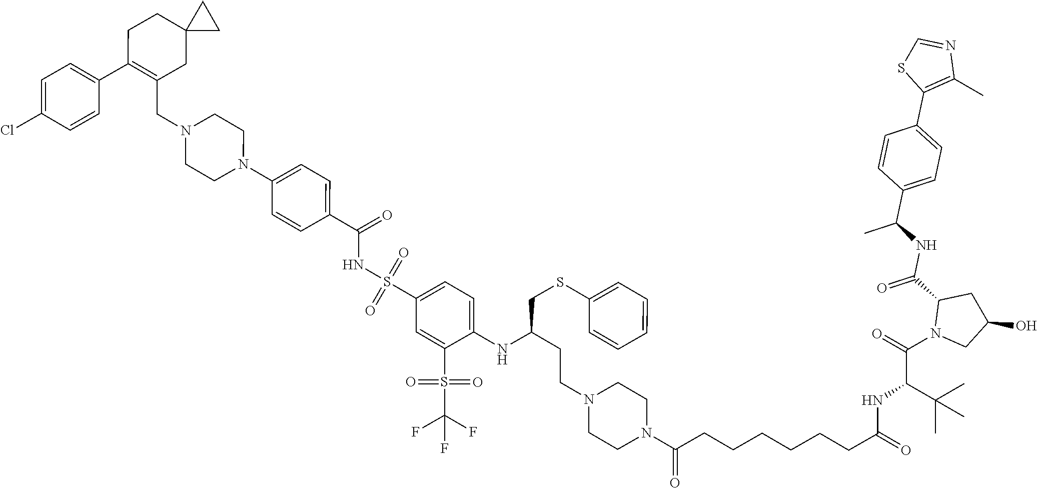 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC7(CC7)C6)CC5)cc4)cc3S(=O)(=O)C(F)(F)F)CC2)C(C)(C)C)cc1]

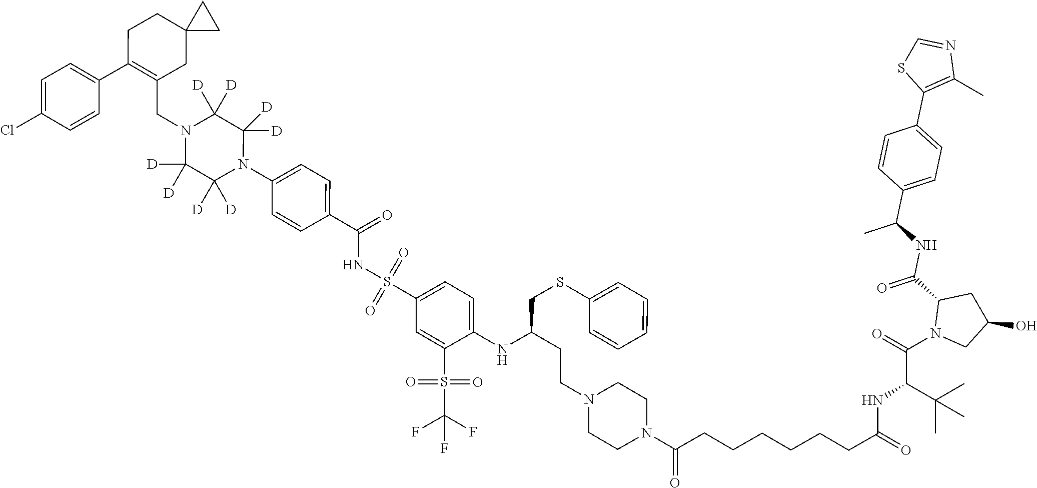 [SMILES: [2H]C1([2H])N(CC2=C(c3ccc(Cl)cc3)CCC3(CC3)C2)C([2H])([2H])C([2H])([2H])N(c2ccc(C(=O)NS(=O)(=O)c3ccc(N[C@H](CCN4CCN(C(=O)CCCCCCC(=O)N[C@H](C(=O)N5C[C@H](O)C[C@H]5C(=O)N[C@@H](C)c5ccc(-c6scnc6C)cc5)C(C)(C)C)CC4)CSc4ccccc4)c(S(=O)(=O)C(F)(F)F)c3)cc2)C1([2H])[2H]]

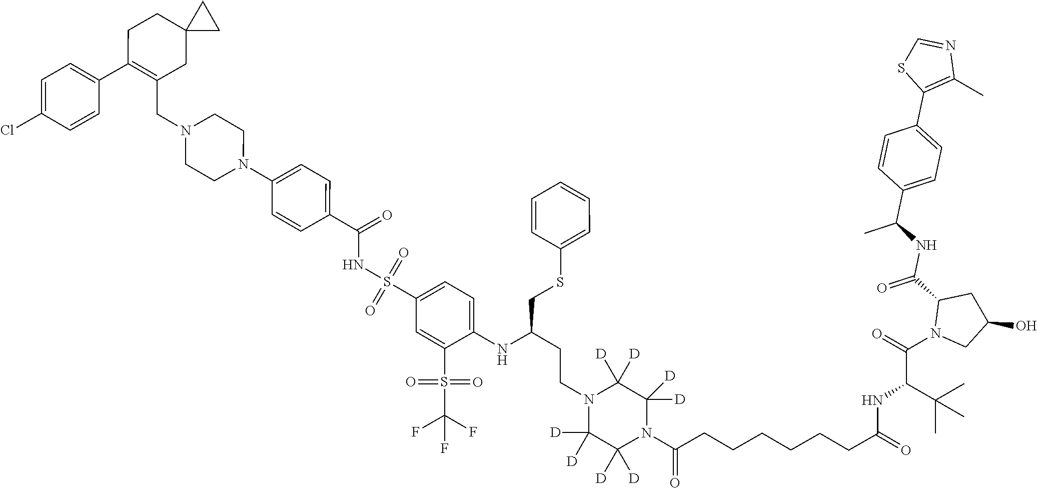 [SMILES: [2H]C1([2H])N(CC[C@H](CSc2ccccc2)Nc2ccc(S(=O)(=O)NC(=O)c3ccc(N4CCN(CC5=C(c6ccc(Cl)cc6)CCC6(CC6)C5)CC4)cc3)cc2S(=O)(=O)C(F)(F)F)C([2H])([2H])C([2H])([2H])N(C(=O)CCCCCCC(=O)N[C@H](C(=O)N2C[C@H](O)C[C@H]2C(=O)N[C@@H](C)c2ccc(-c3scnc3C)cc2)C(C)(C)C)C1([2H])[2H]]

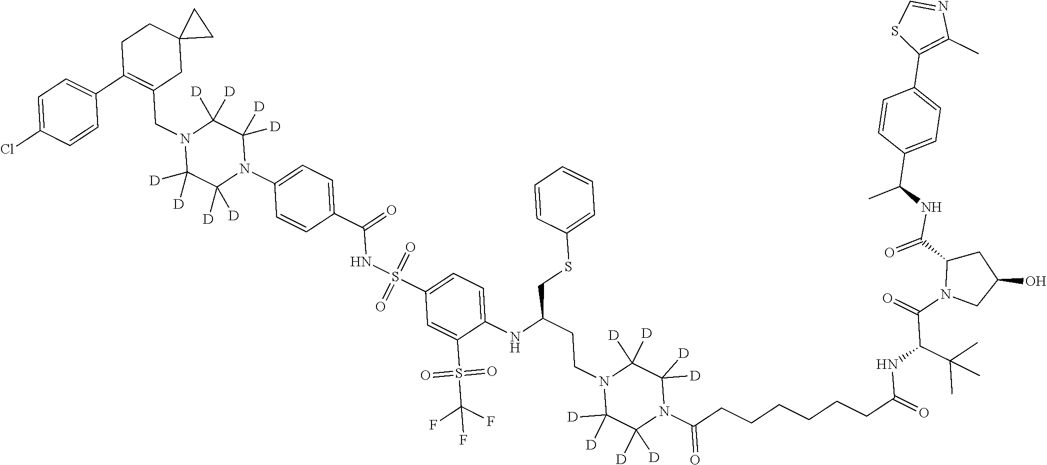 [SMILES: [2H]C1([2H])N(CC[C@H](CSc2ccccc2)Nc2ccc(S(=O)(=O)NC(=O)c3ccc(N4C([2H])([2H])C([2H])([2H])N(CC5=C(c6ccc(Cl)cc6)CCC6(CC6)C5)C([2H])([2H])C4([2H])[2H])cc3)cc2S(=O)(=O)C(F)(F)F)C([2H])([2H])C([2H])([2H])N(C(=O)CCCCCCC(=O)N[C@H](C(=O)N2C[C@H](O)C[C@H]2C(=O)N[C@@H](C)c2ccc(-c3scnc3C)cc2)C(C)(C)C)C1([2H])[2H]]

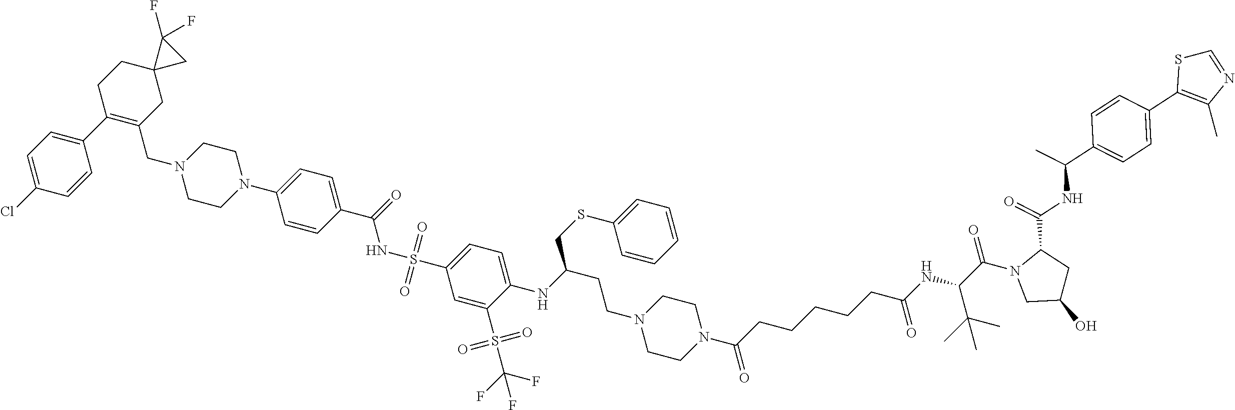 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC7(C6)CC7(F)F)CC5)cc4)cc3S(=O)(=O)C(F)(F)F)CC2)C(C)(C)C)cc1]

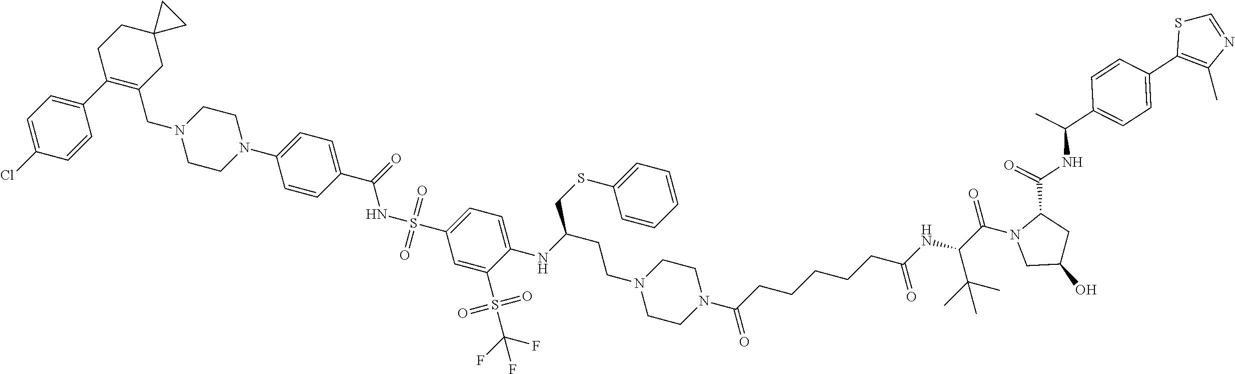 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC7(CC7)C6)CC5)cc4)cc3S(=O)(=O)C(F)(F)F)CC2)C(C)(C)C)cc1]

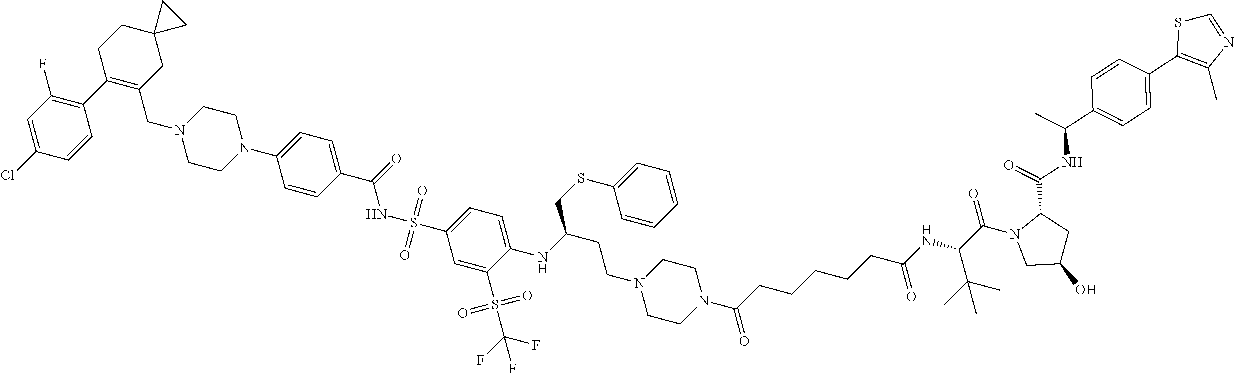 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7F)CCC7(CC7)C6)CC5)cc4)cc3S(=O)(=O)C(F)(F)F)CC2)C(C)(C)C)cc1]

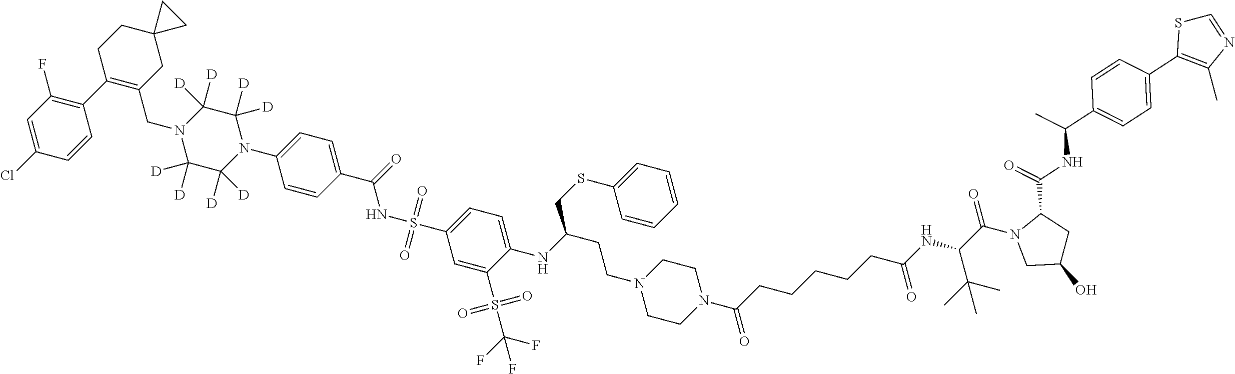 [SMILES: [2H]C1([2H])N(CC2=C(c3ccc(Cl)cc3F)CCC3(CC3)C2)C([2H])([2H])C([2H])([2H])N(c2ccc(C(=O)NS(=O)(=O)c3ccc(N[C@H](CCN4CCN(C(=O)CCCCCC(=O)N[C@H](C(=O)N5C[C@H](O)C[C@H]5C(=O)N[C@@H](C)c5ccc(-c6scnc6C)cc5)C(C)(C)C)CC4)CSc4ccccc4)c(S(=O)(=O)C(F)(F)F)c3)cc2)C1([2H])[2H]]

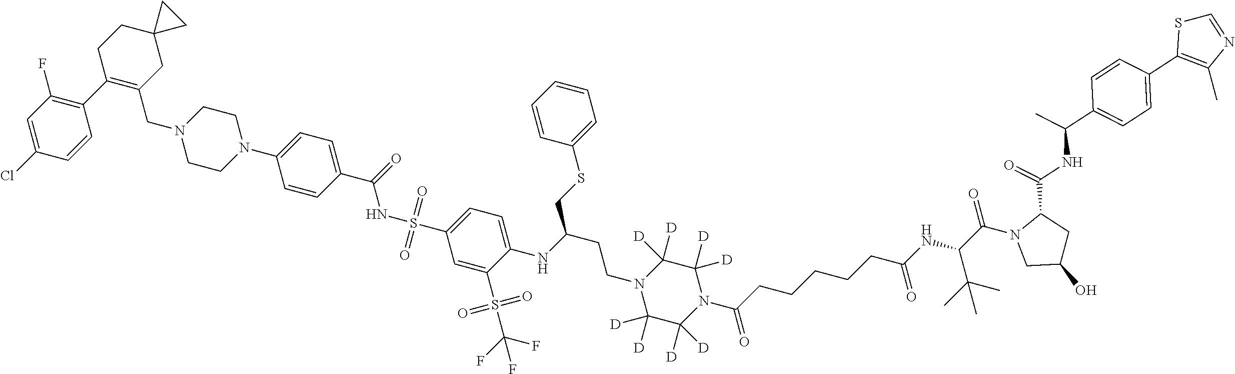 [SMILES: [2H]C1([2H])N(CC[C@H](CSc2ccccc2)Nc2ccc(S(=O)(=O)NC(=O)c3ccc(N4CCN(CC5=C(c6ccc(Cl)cc6F)CCC6(CC6)C5)CC4)cc3)cc2S(=O)(=O)C(F)(F)F)C([2H])([2H])C([2H])([2H])N(C(=O)CCCCCC(=O)N[C@H](C(=O)N2C[C@H](O)C[C@H]2C(=O)N[C@@H](C)c2ccc(-c3scnc3C)cc2)C(C)(C)C)C1([2H])[2H]]

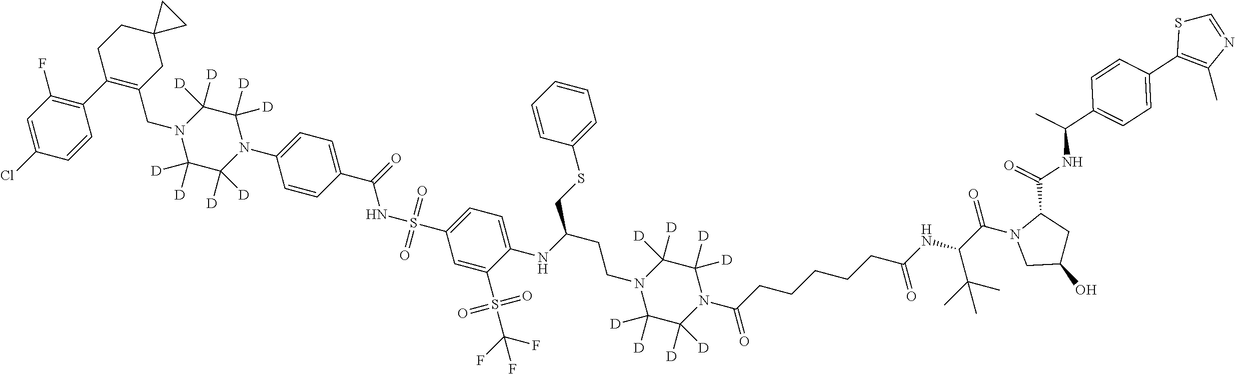 [SMILES: [2H]C1([2H])N(CC[C@H](CSc2ccccc2)Nc2ccc(S(=O)(=O)NC(=O)c3ccc(N4C([2H])([2H])C([2H])([2H])N(CC5=C(c6ccc(Cl)cc6F)CCC6(CC6)C5)C([2H])([2H])C4([2H])[2H])cc3)cc2S(=O)(=O)C(F)(F)F)C([2H])([2H])C([2H])([2H])N(C(=O)CCCCCC(=O)N[C@H](C(=O)N2C[C@H](O)C[C@H]2C(=O)N[C@@H](C)c2ccc(-c3scnc3C)cc2)C(C)(C)C)C1([2H])[2H]]

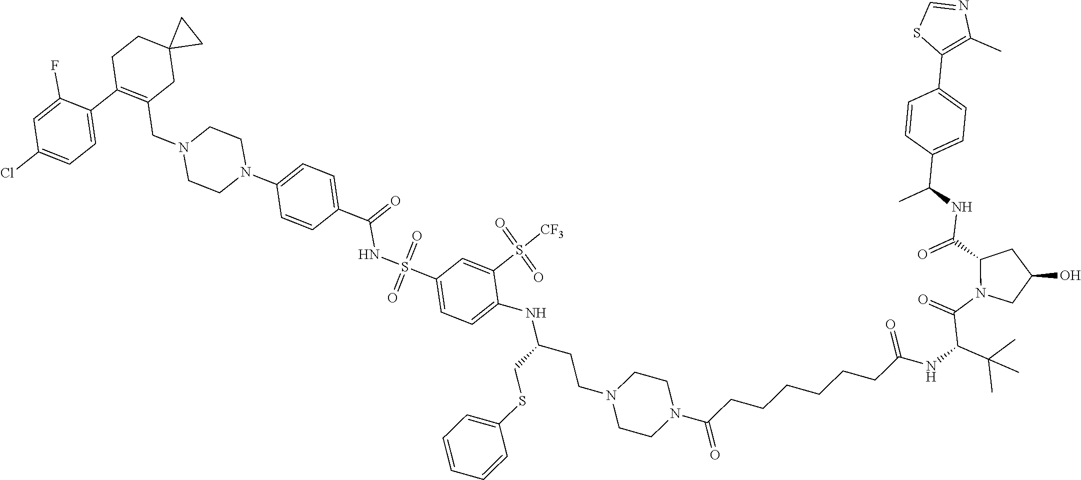 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC[C@H](CSc3ccccc3)Nc3ccc(S(=O)(=O)NC(=O)c4ccc(N5CCN(CC6=C(c7ccc(Cl)cc7F)CCC7(CC7)C6)CC5)cc4)cc3S(=O)(=O)C(F)(F)F)CC2)C(C)(C)C)cc1]

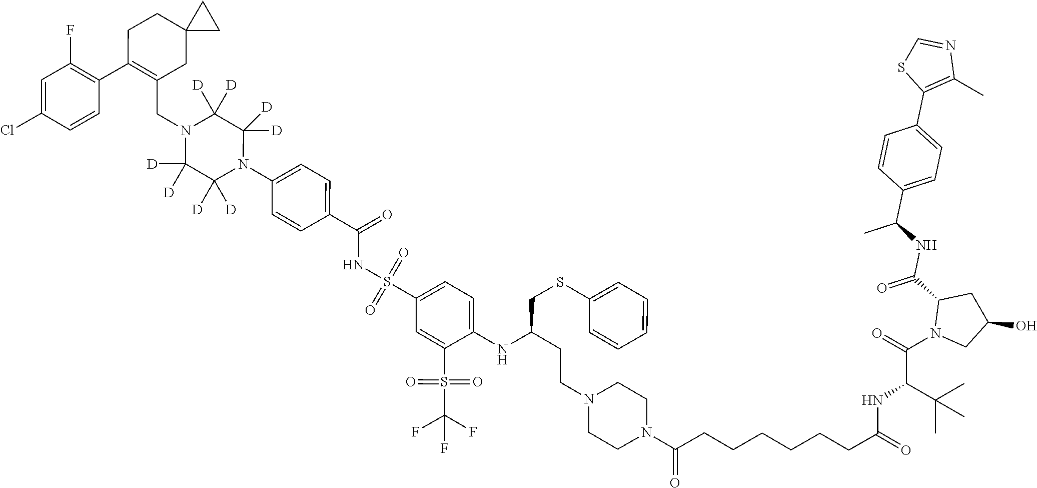 [SMILES: [2H]C1([2H])N(CC2=C(c3ccc(Cl)cc3F)CCC3(CC3)C2)C([2H])([2H])C([2H])([2H])N(c2ccc(C(=O)NS(=O)(=O)c3ccc(N[C@H](CCN4CCN(C(=O)CCCCCCC(=O)N[C@H](C(=O)N5C[C@H](O)C[C@H]5C(=O)N[C@@H](C)c5ccc(-c6scnc6C)cc5)C(C)(C)C)CC4)CSc4ccccc4)c(S(=O)(=O)C(F)(F)F)c3)cc2)C1([2H])[2H]]

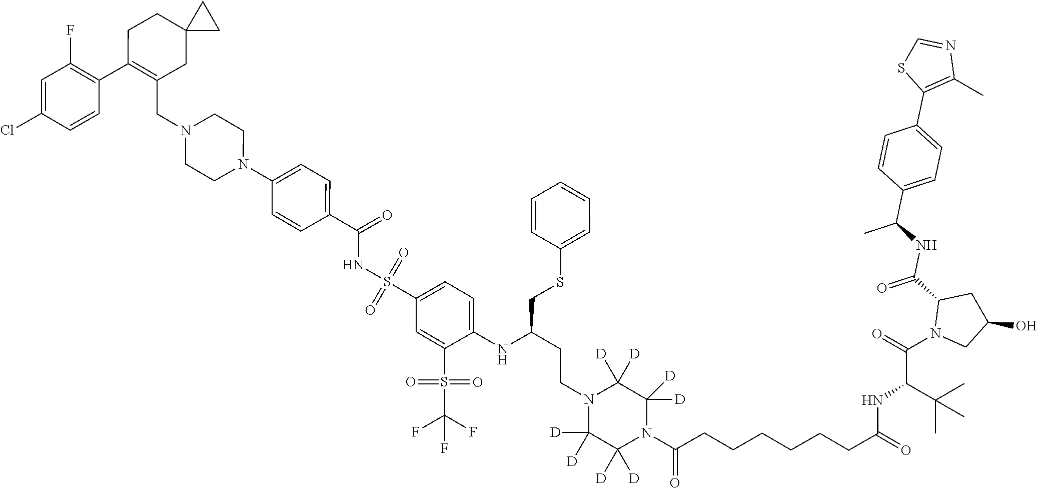 [SMILES: [2H]C1([2H])N(CC[C@H](CSc2ccccc2)Nc2ccc(S(=O)(=O)NC(=O)c3ccc(N4CCN(CC5=C(c6ccc(Cl)cc6F)CCC6(CC6)C5)CC4)cc3)cc2S(=O)(=O)C(F)(F)F)C([2H])([2H])C([2H])([2H])N(C(=O)CCCCCCC(=O)N[C@H](C(=O)N2C[C@H](O)C[C@H]2C(=O)N[C@@H](C)c2ccc(-c3scnc3C)cc2)C(C)(C)C)C1([2H])[2H]]

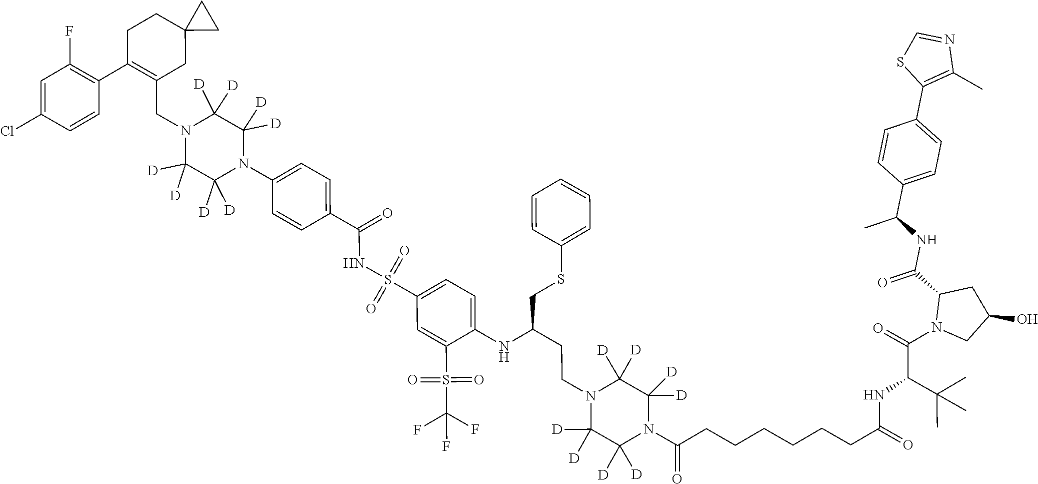 [SMILES: [2H]C1([2H])N(CC[C@H](CSc2ccccc2)Nc2ccc(S(=O)(=O)NC(=O)c3ccc(N4C([2H])([2H])C([2H])([2H])N(CC5=C(c6ccc(Cl)cc6F)CCC6(CC6)C5)C([2H])([2H])C4([2H])[2H])cc3)cc2S(=O)(=O)C(F)(F)F)C([2H])([2H])C([2H])([2H])N(C(=O)CCCCCCC(=O)N[C@H](C(=O)N2C[C@H](O)C[C@H]2C(=O)N[C@@H](C)c2ccc(-c3scnc3C)cc2)C(C)(C)C)C1([2H])[2H]]

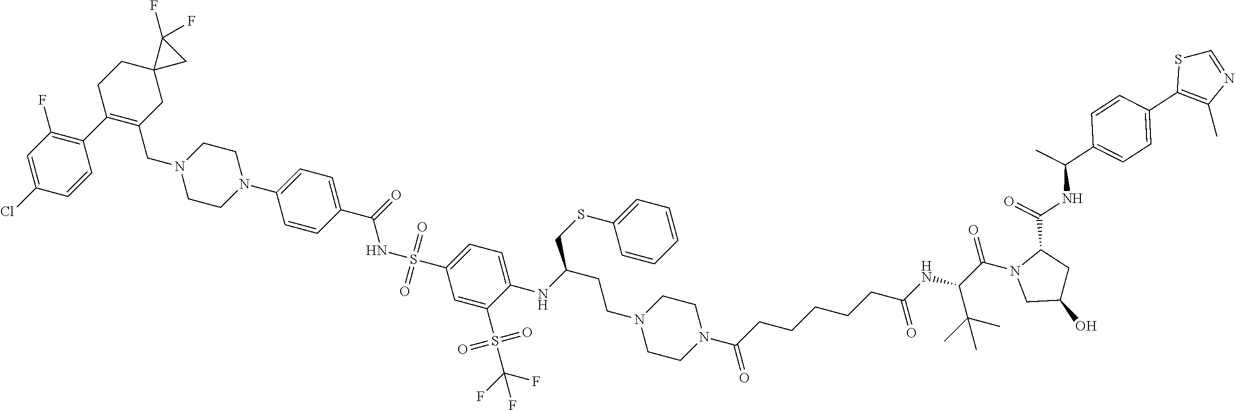 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7F)CCC7(C6)CC7(F)F)CC5)cc4)cc3S(=O)(=O)C(F)(F)F)CC2)C(C)(C)C)cc1]

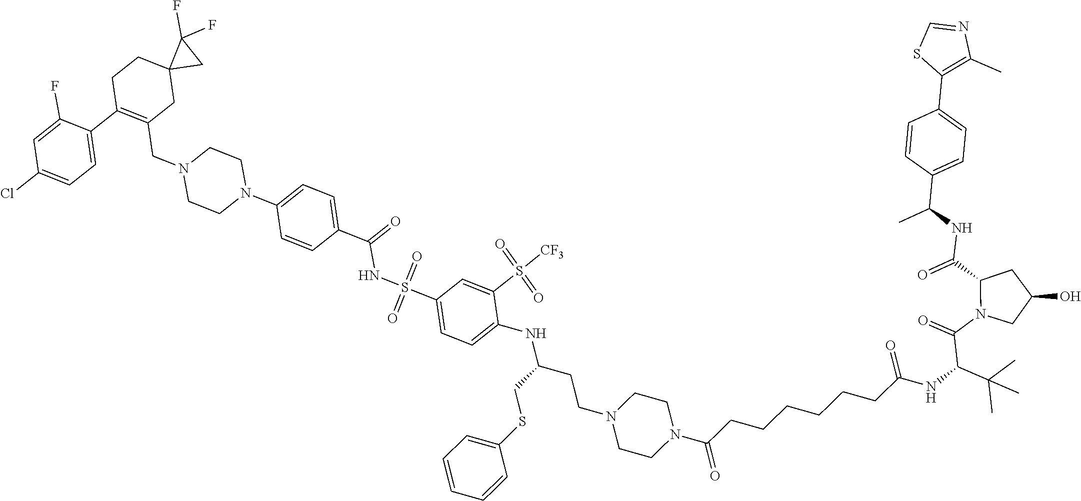 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC[C@H](CSc3ccccc3)Nc3ccc(S(=O)(=O)NC(=O)c4ccc(N5CCN(CC6=C(c7ccc(Cl)cc7F)CCC7(C6)CC7(F)F)CC5)cc4)cc3S(=O)(=O)C(F)(F)F)CC2)C(C)(C)C)cc1]

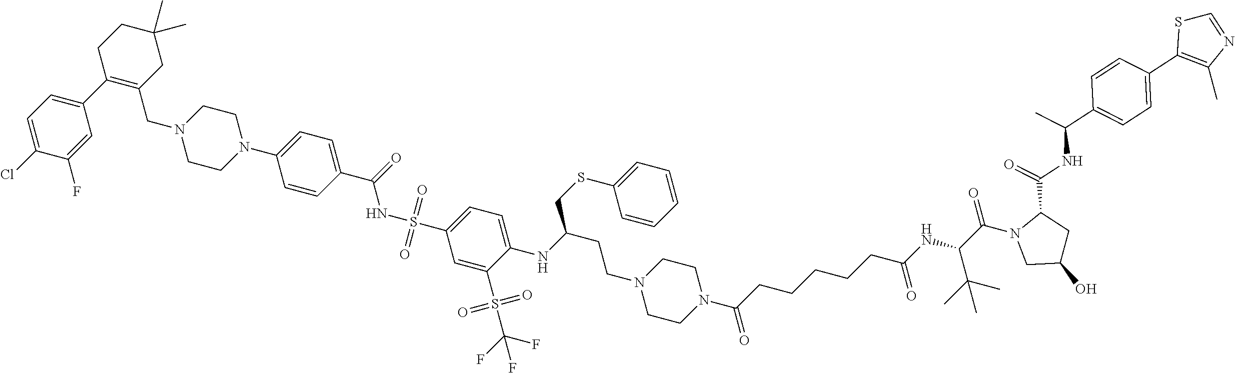 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)c(F)c7)CCC(C)(C)C6)CC5)cc4)cc3S(=O)(=O)C(F)(F)F)CC2)C(C)(C)C)cc1]

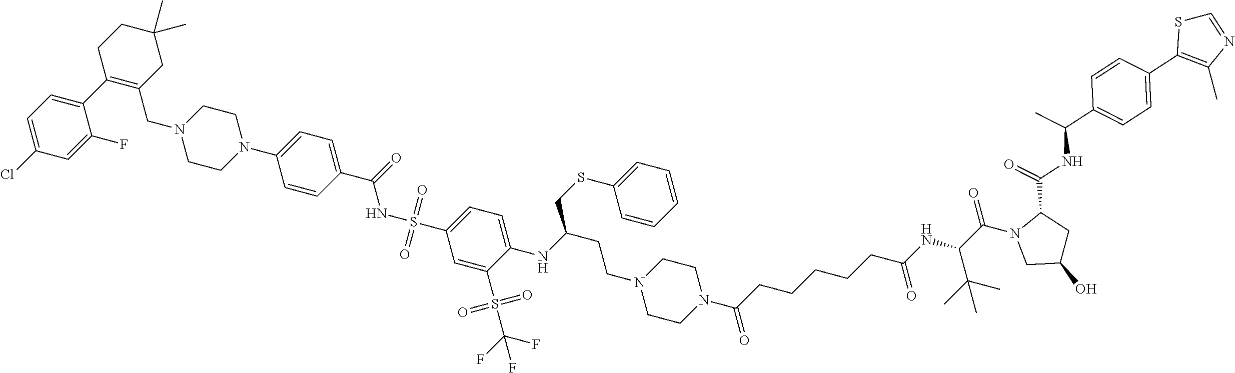 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7F)CCC(C)(C)C6)CC5)cc4)cc3S(=O)(=O)C(F)(F)F)CC2)C(C)(C)C)cc1]

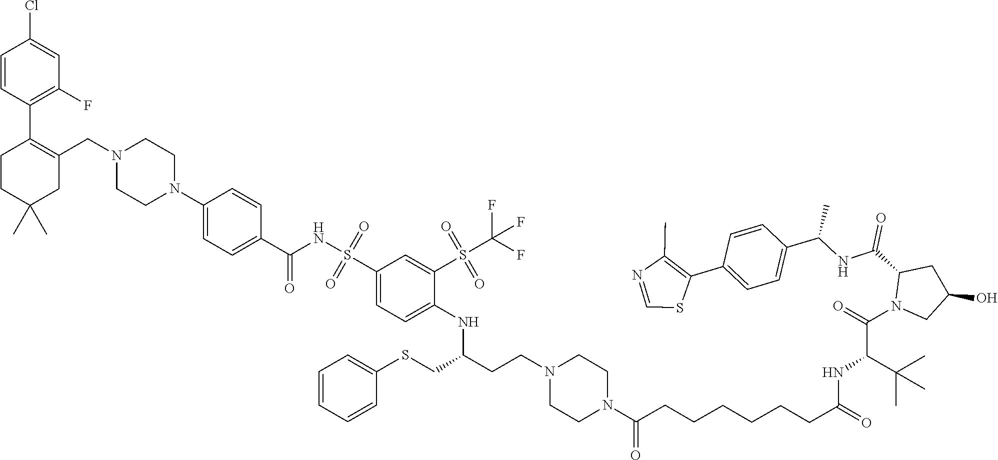 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC[C@H](CSc3ccccc3)Nc3ccc(S(=O)(=O)NC(=O)c4ccc(N5CCN(CC6=C(c7ccc(Cl)cc7F)CCC(C)(C)C6)CC5)cc4)cc3S(=O)(=O)C(F)(F)F)CC2)C(C)(C)C)cc1]

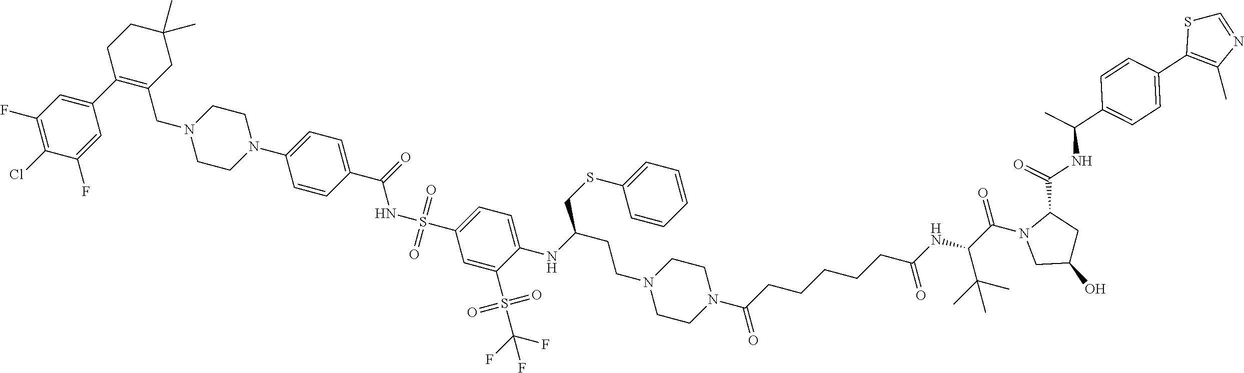 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7cc(F)c(Cl)c(F)c7)CCC(C)(C)C6)CC5)cc4)cc3S(=O)(=O)C(F)(F)F)CC2)C(C)(C)C)cc1]

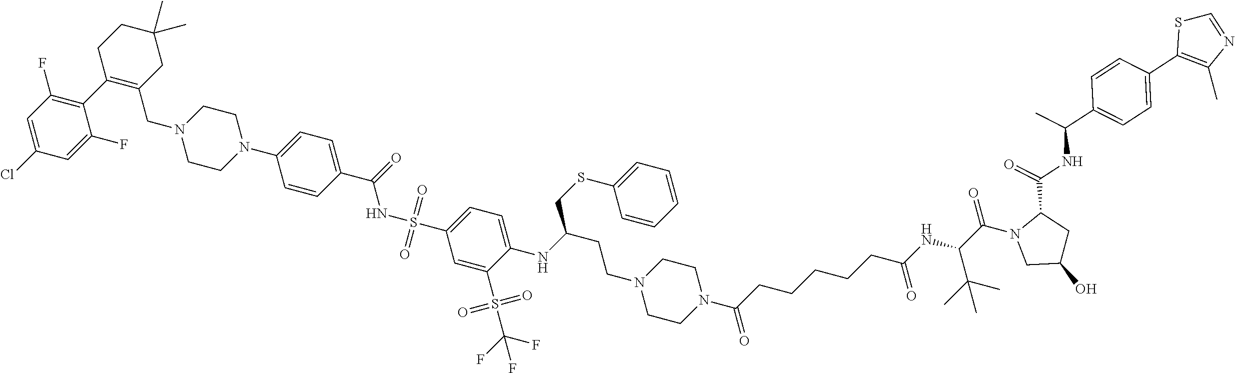 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7c(F)cc(Cl)cc7F)CCC(C)(C)C6)CC5)cc4)cc3S(=O)(=O)C(F)(F)F)CC2)C(C)(C)C)cc1]

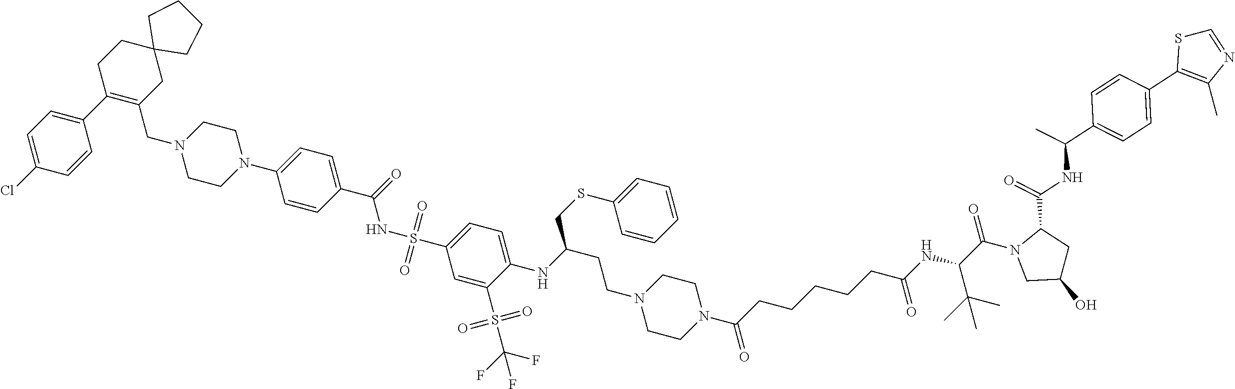 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC7(CCCC7)C6)CC5)cc4)cc3S(=O)(=O)C(F)(F)F)CC2)C(C)(C)C)cc1]

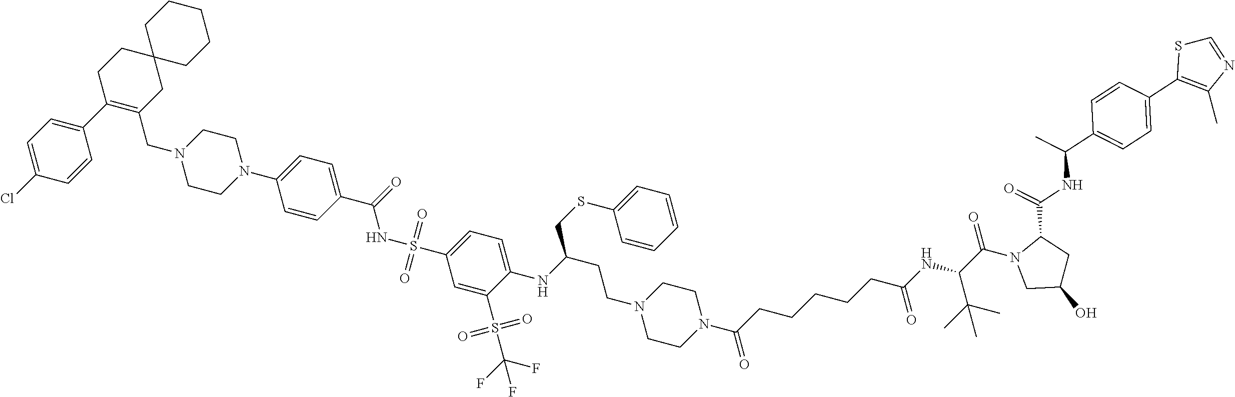 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC7(CCCCC7)C6)CC5)cc4)cc3S(=O)(=O)C(F)(F)F)CC2)C(C)(C)C)cc1]

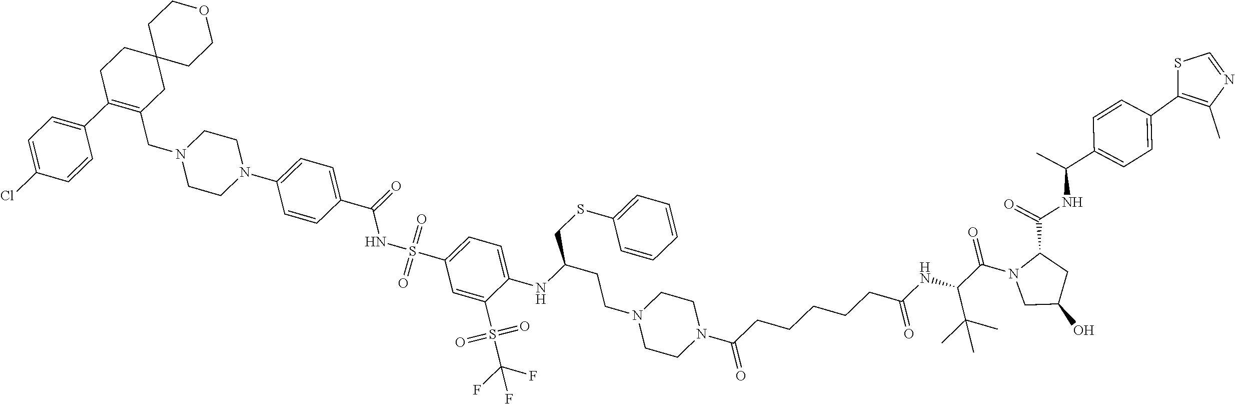 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC7(CCOCC7)C6)CC5)cc4)cc3S(=O)(=O)C(F)(F)F)CC2)C(C)(C)C)cc1]

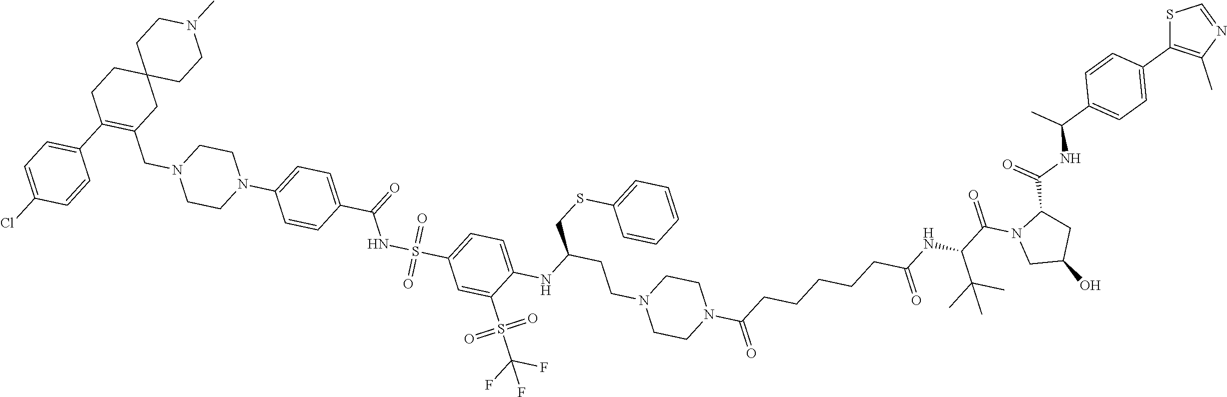 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC7(CCN(C)CC7)C6)CC5)cc4)cc3S(=O)(=O)C(F)(F)F)CC2)C(C)(C)C)cc1]

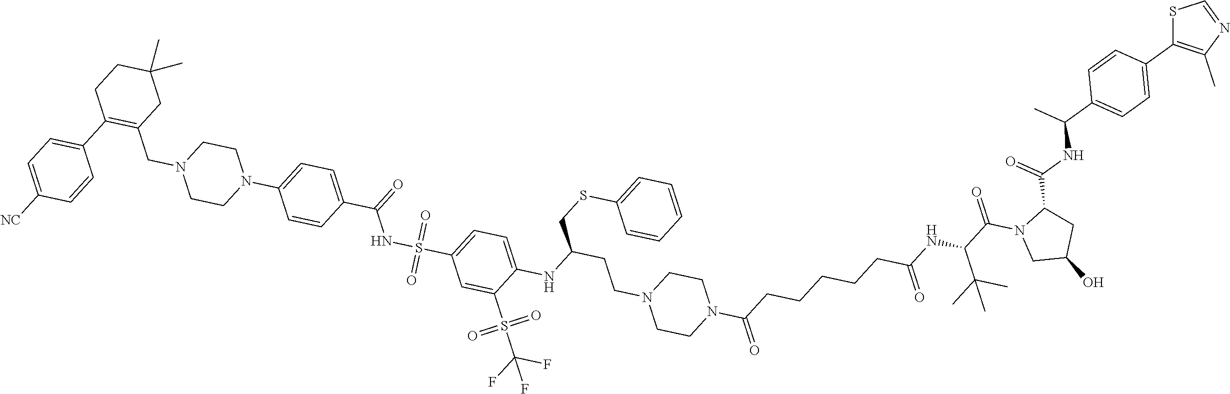 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(C#N)cc7)CCC(C)(C)C6)CC5)cc4)cc3S(=O)(=O)C(F)(F)F)CC2)C(C)(C)C)cc1]

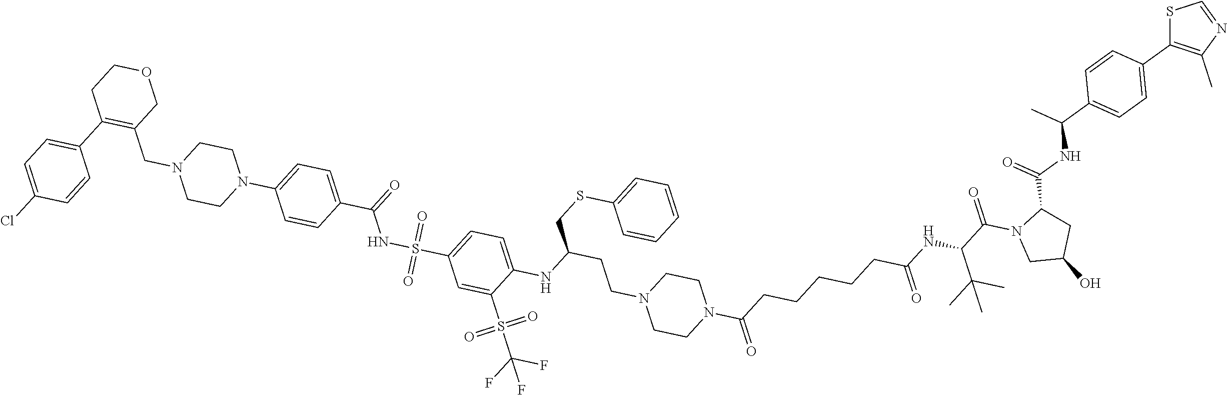 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCOC6)CC5)cc4)cc3S(=O)(=O)C(F)(F)F)CC2)C(C)(C)C)cc1]

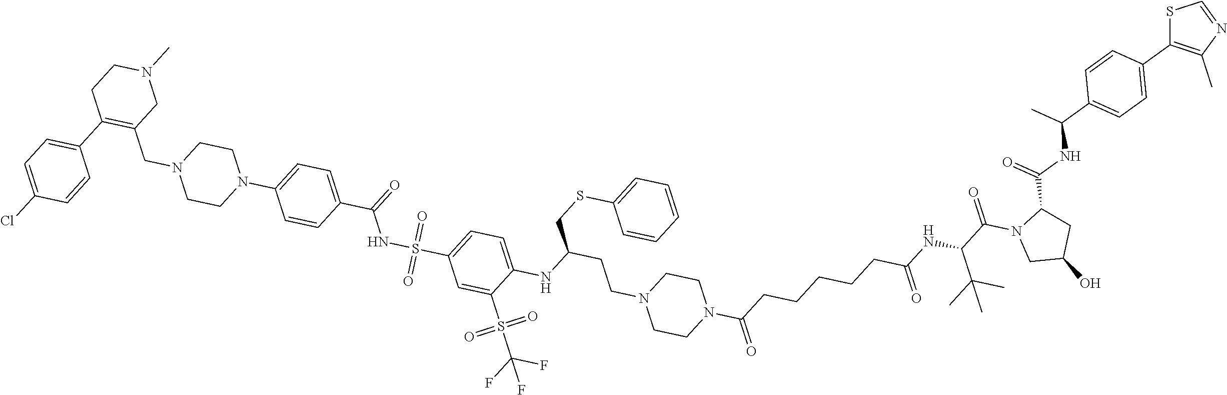 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCN(C)C6)CC5)cc4)cc3S(=O)(=O)C(F)(F)F)CC2)C(C)(C)C)cc1]

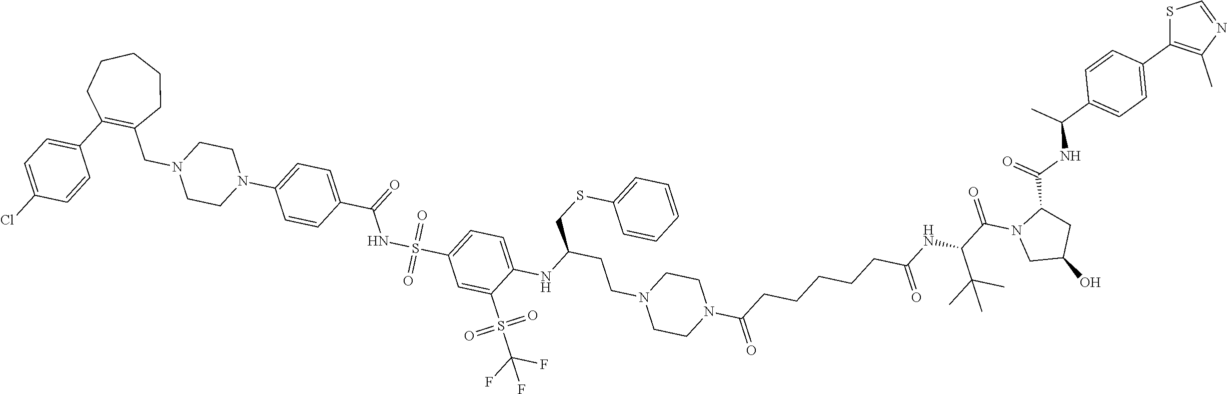 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCCCC6)CC5)cc4)cc3S(=O)(=O)C(F)(F)F)CC2)C(C)(C)C)cc1]

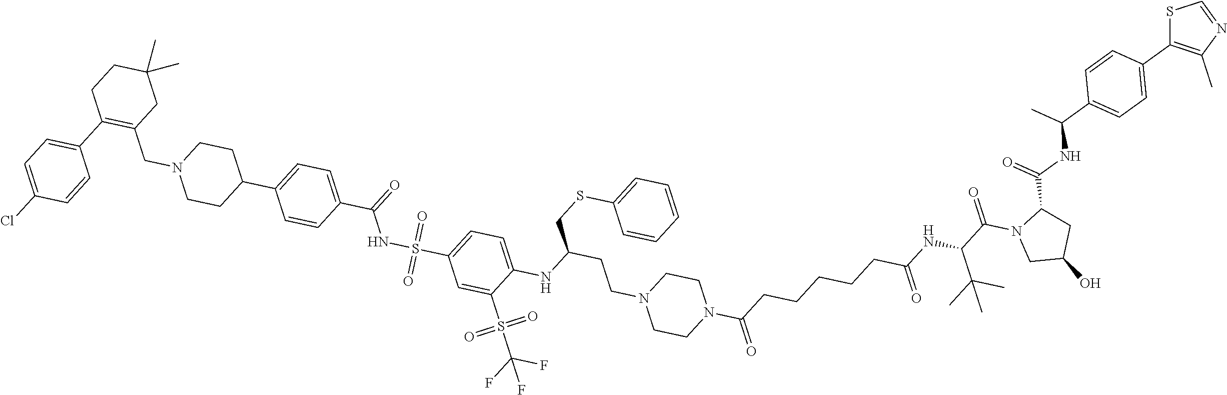 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(C5CCN(CC6=C(c7ccc(Cl)cc7)CCC(C)(C)C6)CC5)cc4)cc3S(=O)(=O)C(F)(F)F)CC2)C(C)(C)C)cc1]

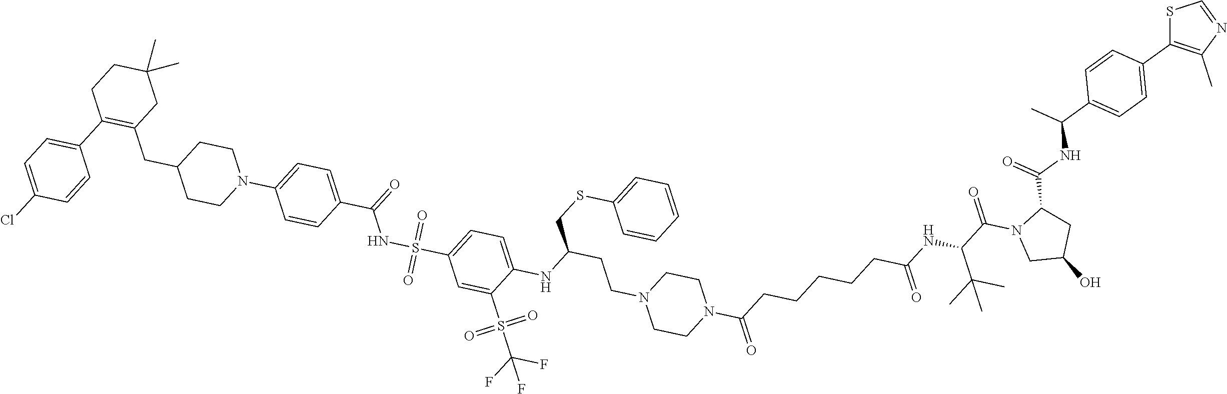 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCC(CC6=C(c7ccc(Cl)cc7)CCC(C)(C)C6)CC5)cc4)cc3S(=O)(=O)C(F)(F)F)CC2)C(C)(C)C)cc1]

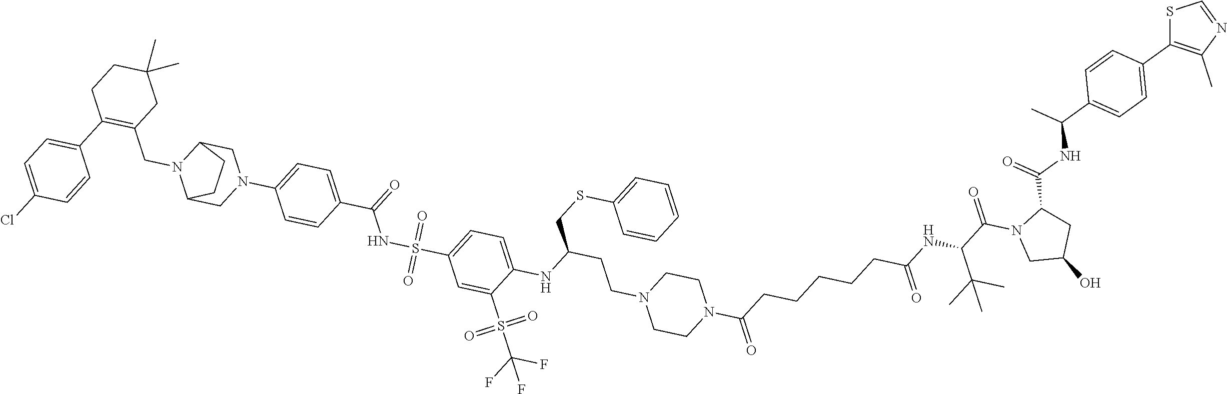 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CC6CCC(C5)N6CC5=C(c6ccc(Cl)cc6)CCC(C)(C)C5)cc4)cc3S(=O)(=O)C(F)(F)F)CC2)C(C)(C)C)cc1]

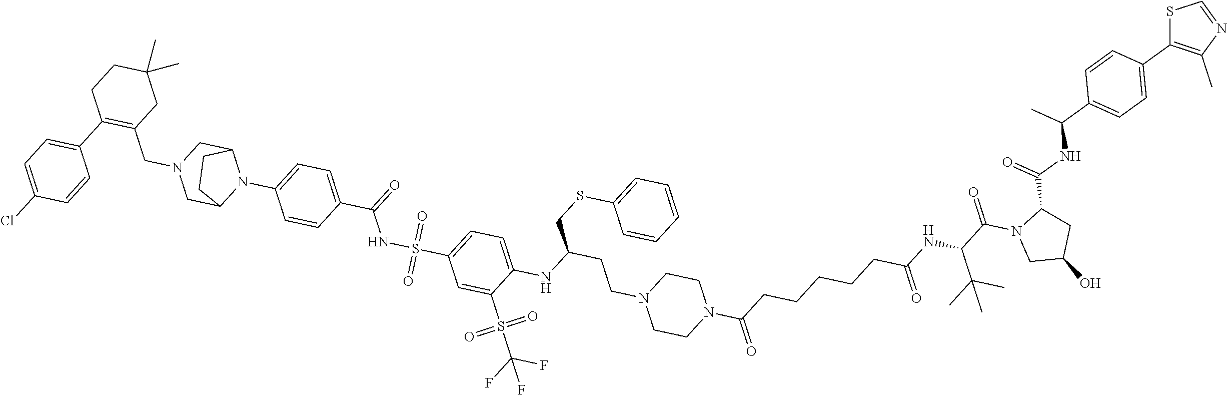 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5C6CCC5CN(CC5=C(c7ccc(Cl)cc7)CCC(C)(C)C5)C6)cc4)cc3S(=O)(=O)C(F)(F)F)CC2)C(C)(C)C)cc1]

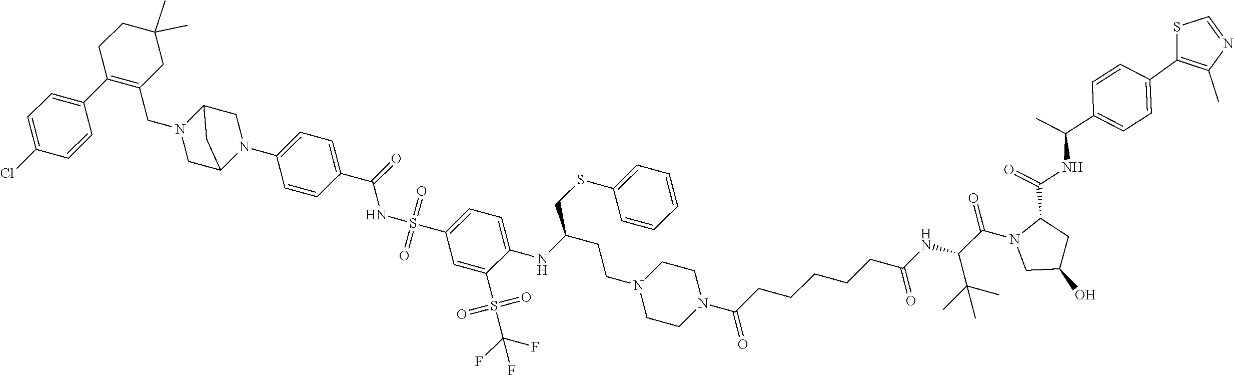 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CC6CC5CN6CC5=C(c6ccc(Cl)cc6)CCC(C)(C)C5)cc4)cc3S(=O)(=O)C(F)(F)F)CC2)C(C)(C)C)cc1]

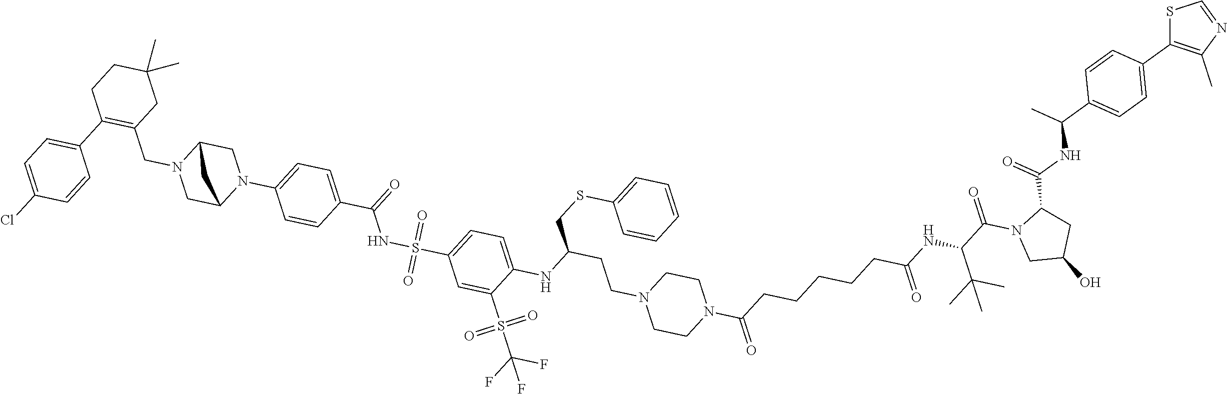 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5C[C@@H]6C[C@H]5CN6CC5=C(c6ccc(Cl)cc6)CCC(C)(C)C5)cc4)cc3S(=O)(=O)C(F)(F)F)CC2)C(C)(C)C)cc1]

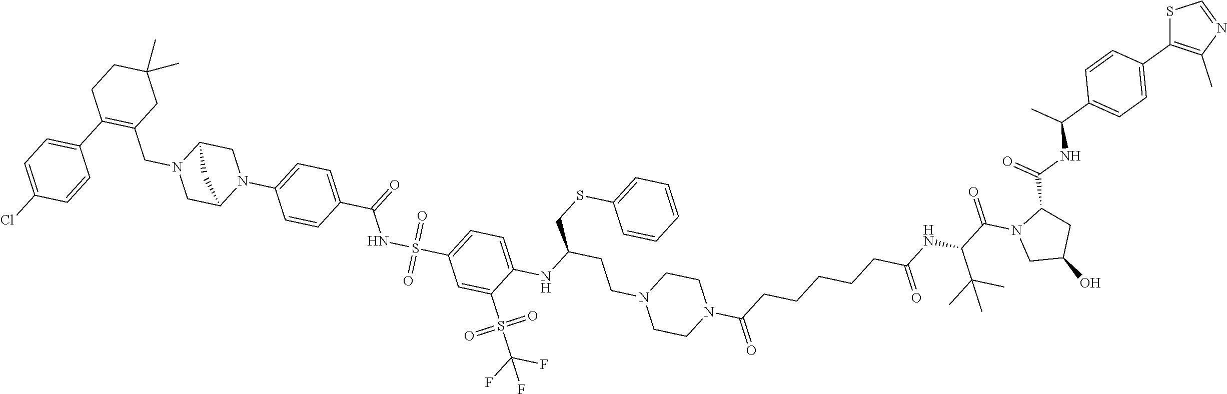 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5C[C@H]6C[C@@H]5CN6CC5=C(c6ccc(Cl)cc6)CCC(C)(C)C5)cc4)cc3S(=O)(=O)C(F)(F)F)CC2)C(C)(C)C)cc1]

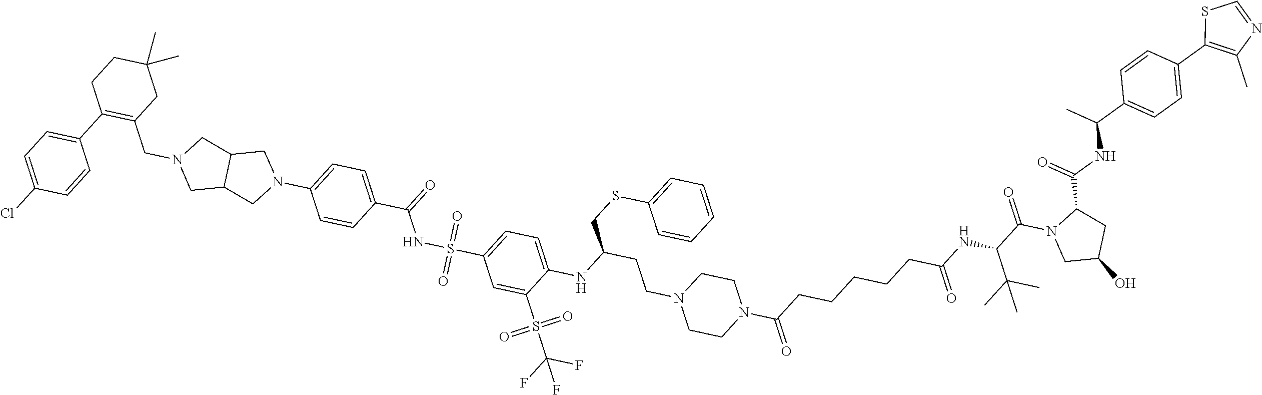 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CC6CN(CC7=C(c8ccc(Cl)cc8)CCC(C)(C)C7)CC6C5)cc4)cc3S(=O)(=O)C(F)(F)F)CC2)C(C)(C)C)cc1]

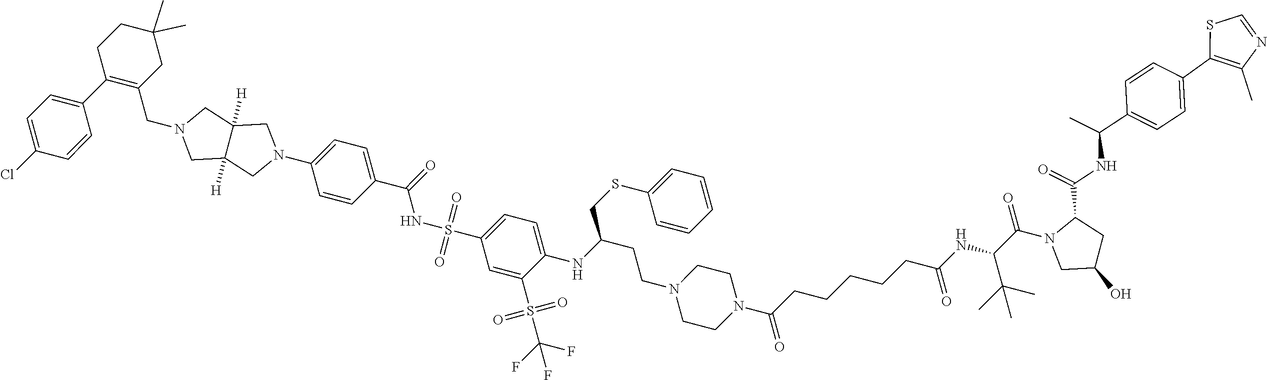 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5C[C@H]6CN(CC7=C(c8ccc(Cl)cc8)CCC(C)(C)C7)C[C@H]6C5)cc4)cc3S(=O)(=O)C(F)(F)F)CC2)C(C)(C)C)cc1]

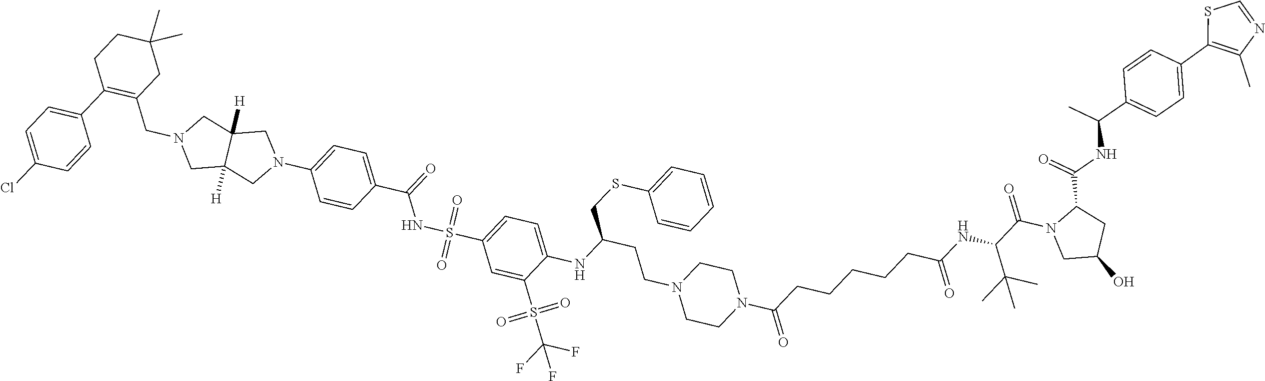 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5C[C@H]6CN(CC7=C(c8ccc(Cl)cc8)CCC(C)(C)C7)C[C@@H]6C5)cc4)cc3S(=O)(=O)C(F)(F)F)CC2)C(C)(C)C)cc1]

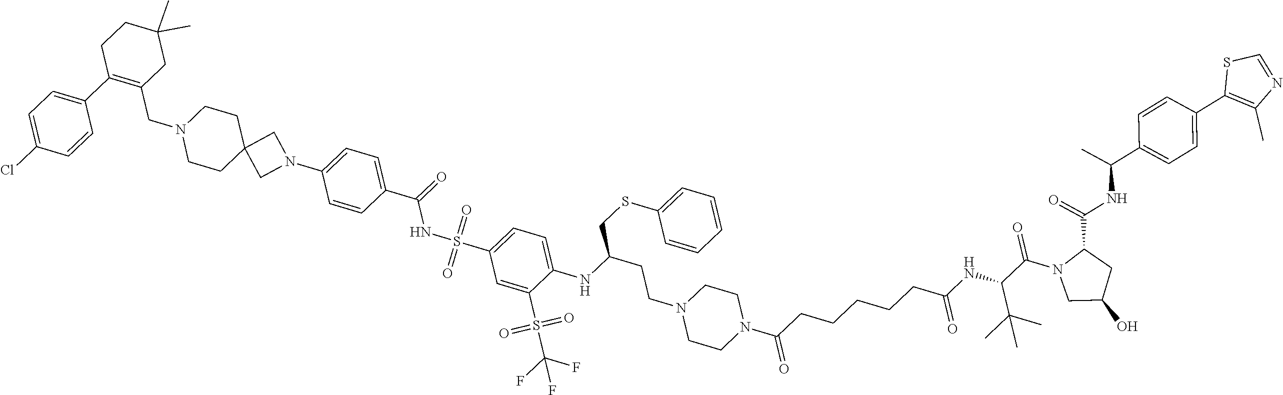 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CC6(CCN(CC7=C(c8ccc(Cl)cc8)CCC(C)(C)C7)CC6)C5)cc4)cc3S(=O)(=O)C(F)(F)F)CC2)C(C)(C)C)cc1]

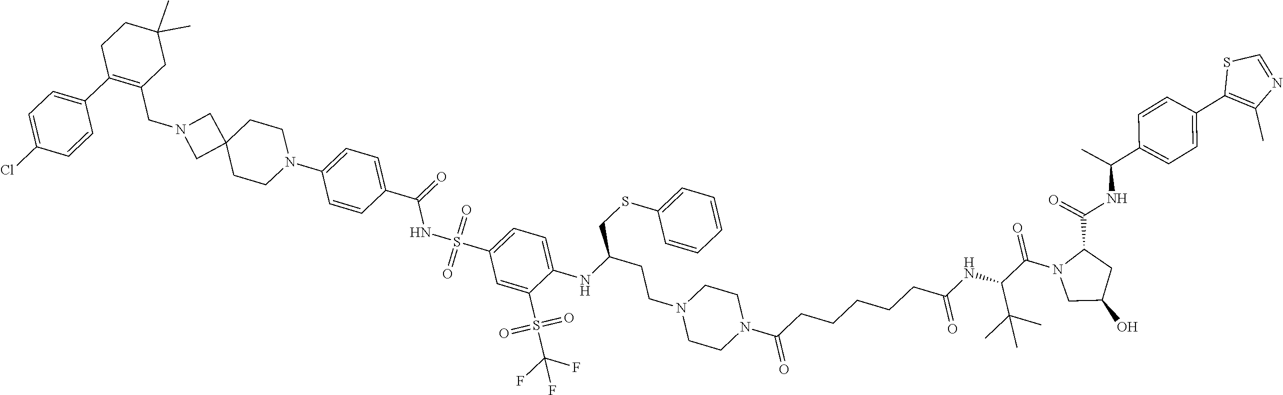 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCC6(CC5)CN(CC5=C(c7ccc(Cl)cc7)CCC(C)(C)C5)C6)cc4)cc3S(=O)(=O)C(F)(F)F)CC2)C(C)(C)C)cc1]

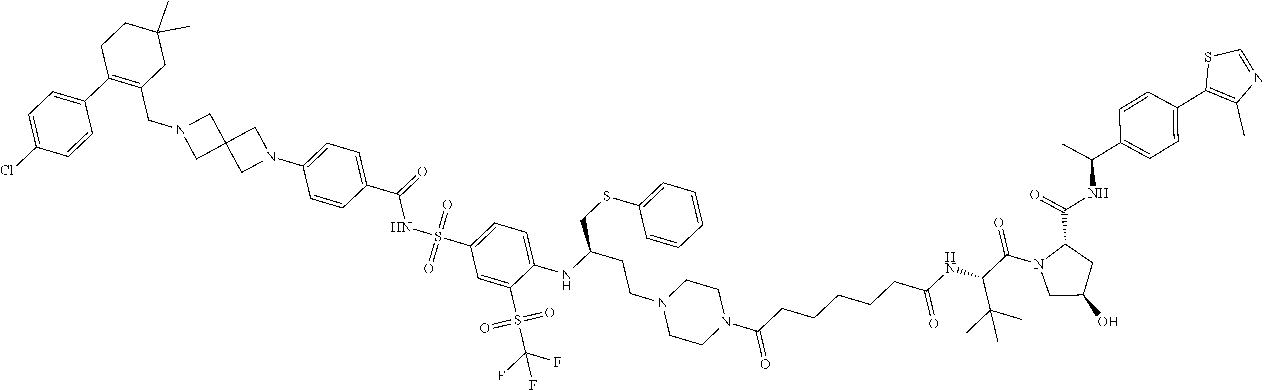 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CC6(CN(CC7=C(c8ccc(Cl)cc8)CCC(C)(C)C7)C6)C5)cc4)cc3S(=O)(=O)C(F)(F)F)CC2)C(C)(C)C)cc1]

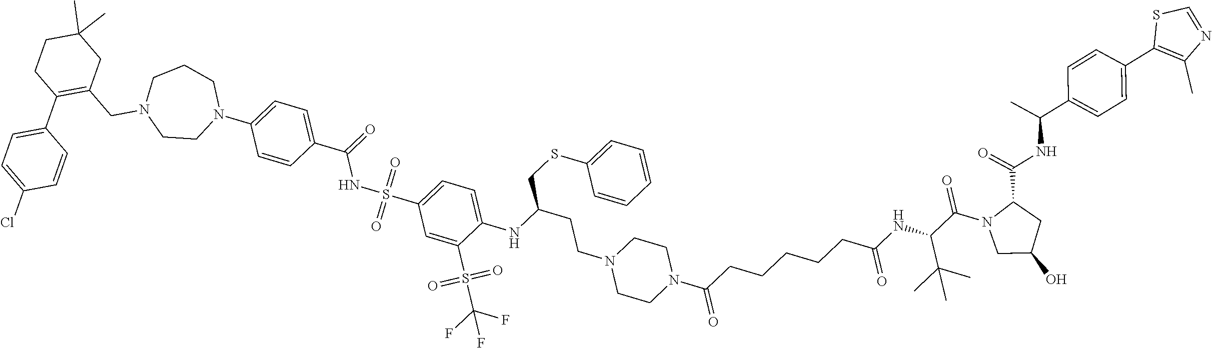 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCCN(CC6=C(c7ccc(Cl)cc7)CCC(C)(C)C6)CC5)cc4)cc3S(=O)(=O)C(F)(F)F)CC2)C(C)(C)C)cc1]

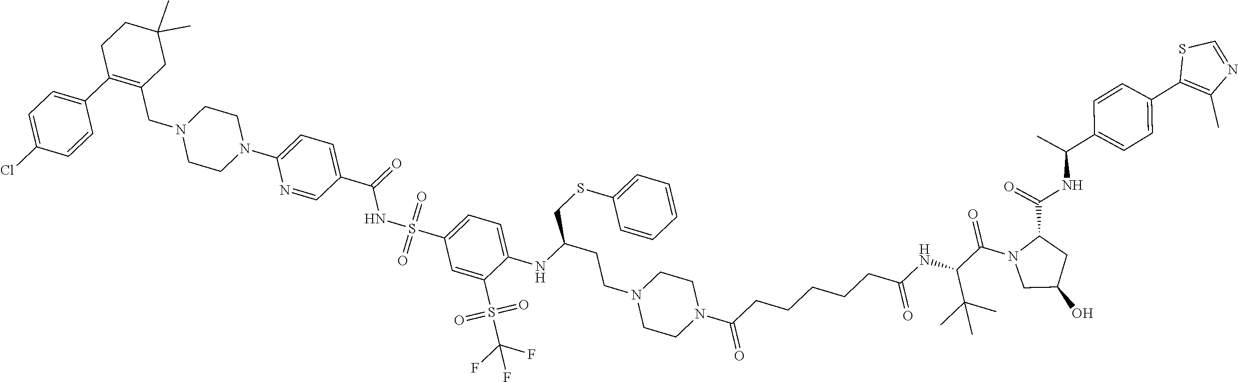 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)nc4)cc3S(=O)(=O)C(F)(F)F)CC2)C(C)(C)C)cc1]

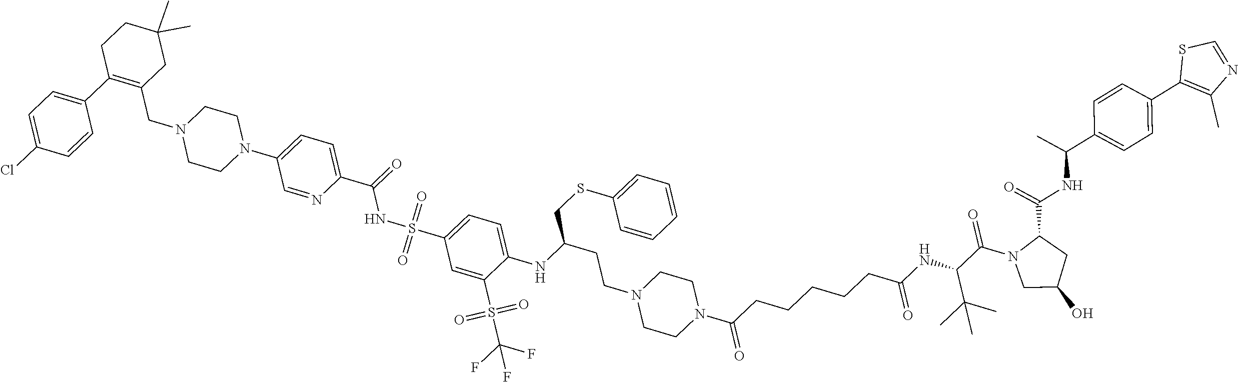 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cn4)cc3S(=O)(=O)C(F)(F)F)CC2)C(C)(C)C)cc1]

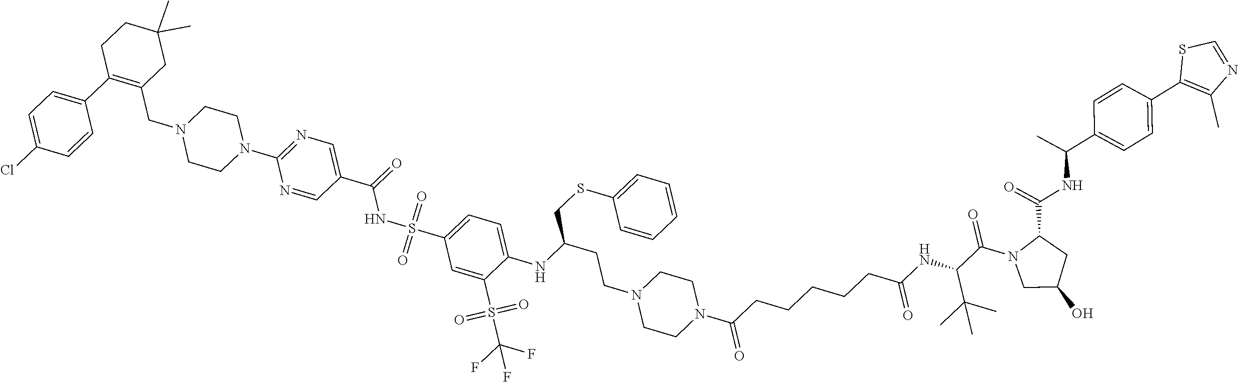 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4cnc(N5CCN(CC6=C(c7ccc(Cl)cc7)CCC(C)(C)C6)CC5)nc4)cc3S(=O)(=O)C(F)(F)F)CC2)C(C)(C)C)cc1]

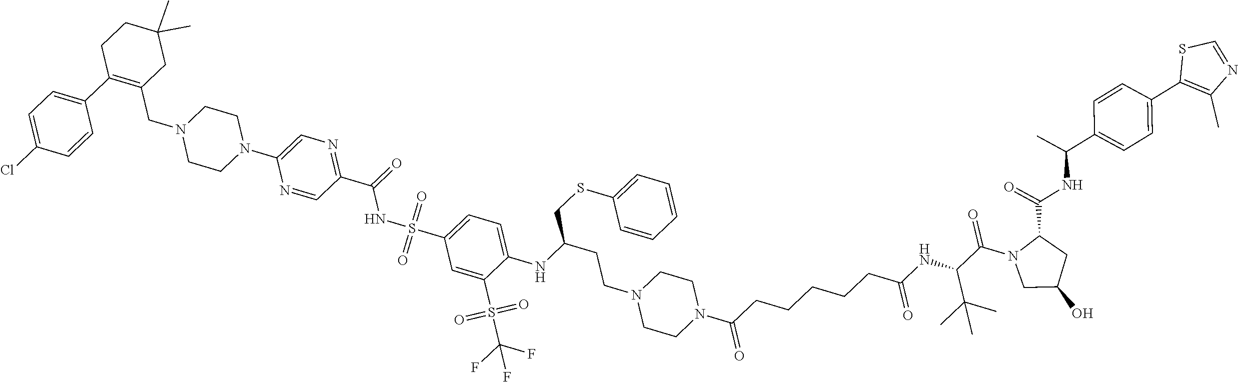 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4cnc(N5CCN(CC6=C(c7ccc(Cl)cc7)CCC(C)(C)C6)CC5)cn4)cc3S(=O)(=O)C(F)(F)F)CC2)C(C)(C)C)cc1]

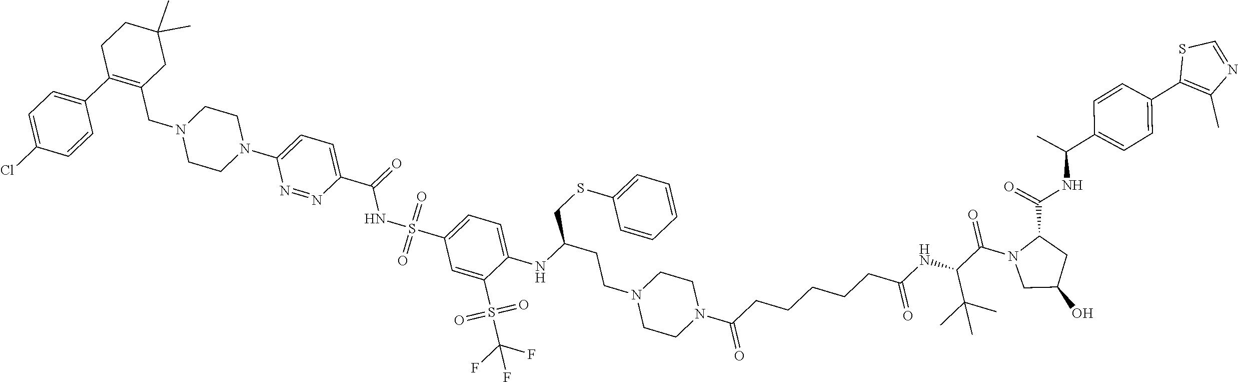 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)nn4)cc3S(=O)(=O)C(F)(F)F)CC2)C(C)(C)C)cc1]

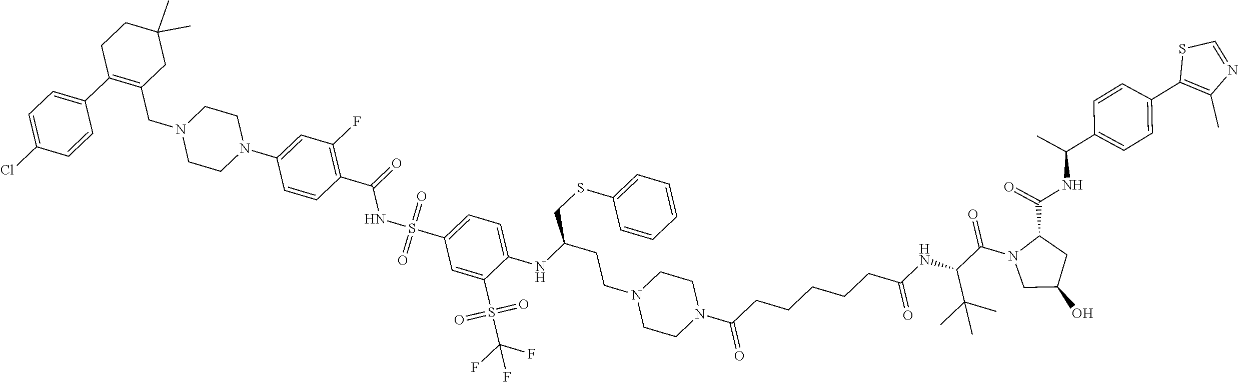 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4F)cc3S(=O)(=O)C(F)(F)F)CC2)C(C)(C)C)cc1]

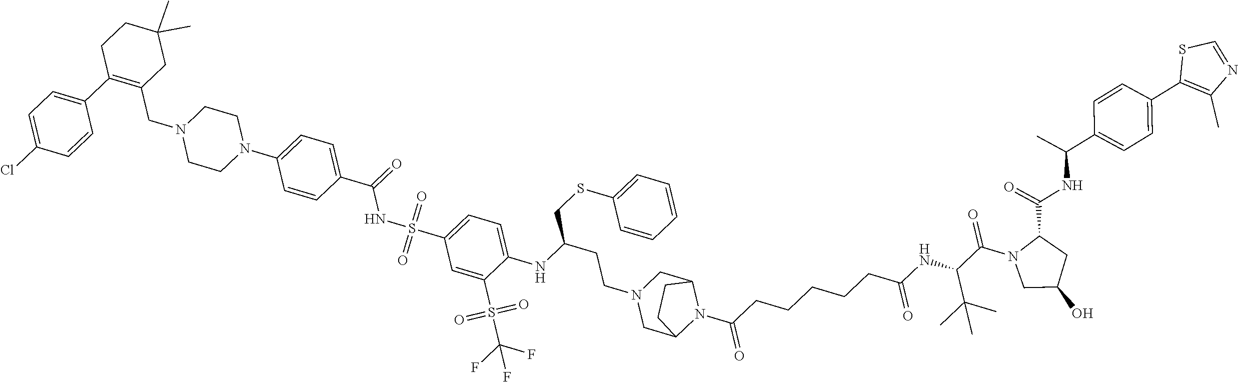 [SMILES: Cc1ncsc1-c1ccc([C@H](C)NC(=O)[C@@H]2C[C@@H](O)CN2C(=O)[C@@H](NC(=O)CCCCCC(=O)N2C3CCC2CN(CC[C@H](CSc2ccccc2)Nc2ccc(S(=O)(=O)NC(=O)c4ccc(N5CCN(CC6=C(c7ccc(Cl)cc7)CCC(C)(C)C6)CC5)cc4)cc2S(=O)(=O)C(F)(F)F)C3)C(C)(C)C)cc1]

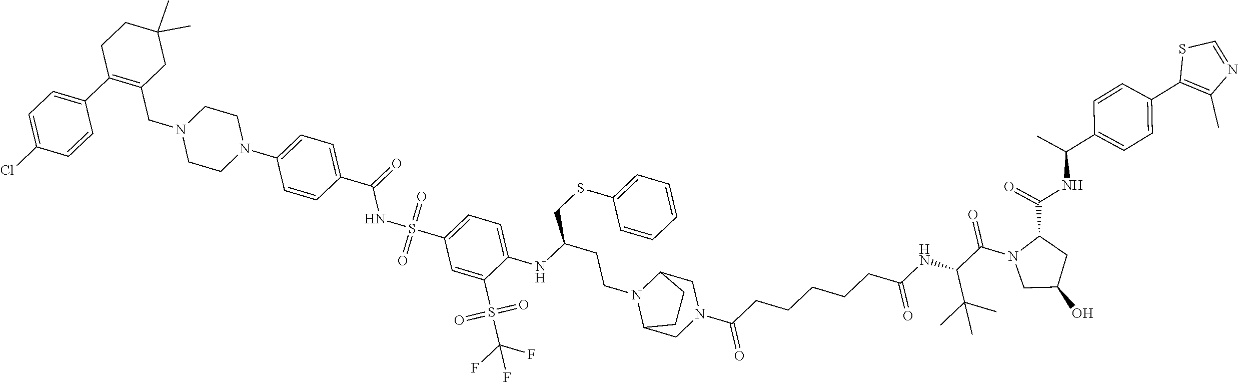 [SMILES: Cc1ncsc1-c1ccc([C@H](C)NC(=O)[C@@H]2C[C@@H](O)CN2C(=O)[C@@H](NC(=O)CCCCCC(=O)N2CC3CCC(C2)N3CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)C(C)(C)C)cc1]

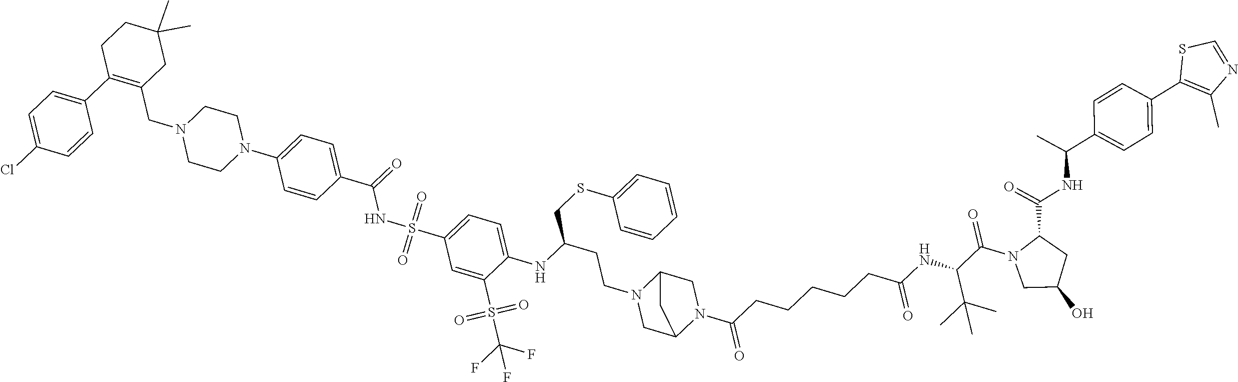 [SMILES: Cc1ncsc1-c1ccc([C@H](C)NC(=O)[C@@H]2C[C@@H](O)CN2C(=O)[C@@H](NC(=O)CCCCCC(=O)N2CC3CC2CN3CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)C(C)(C)C)cc1]

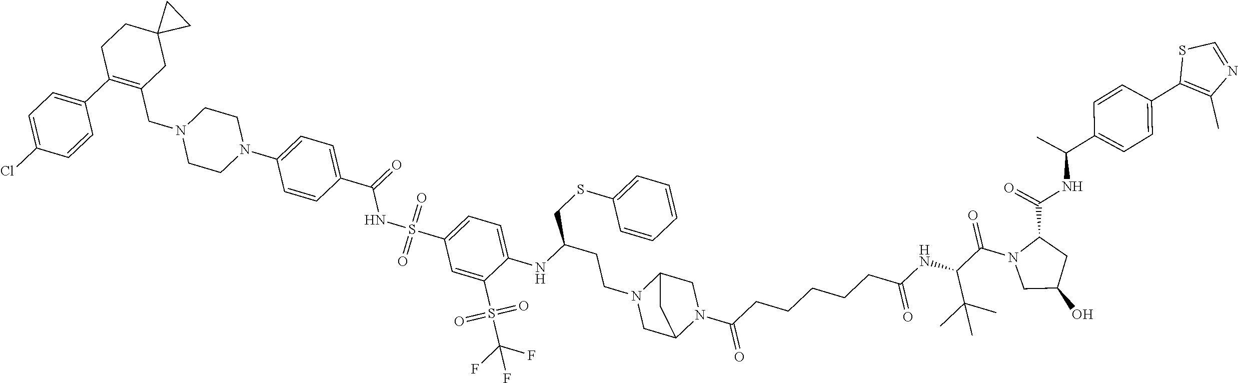 [SMILES: Cc1ncsc1-c1ccc([C@H](C)NC(=O)[C@@H]2C[C@@H](O)CN2C(=O)[C@@H](NC(=O)CCCCCC(=O)N2CC3CC2CN3CC[C@H](CSc2ccccc2)Nc2ccc(S(=O)(=O)NC(=O)c3ccc(N4CCN(CC5=C(c6ccc(Cl)cc6)CCC6(CC6)C5)CC4)cc3)cc2S(=O)(=O)C(F)(F)F)C(C)(C)C)cc1]

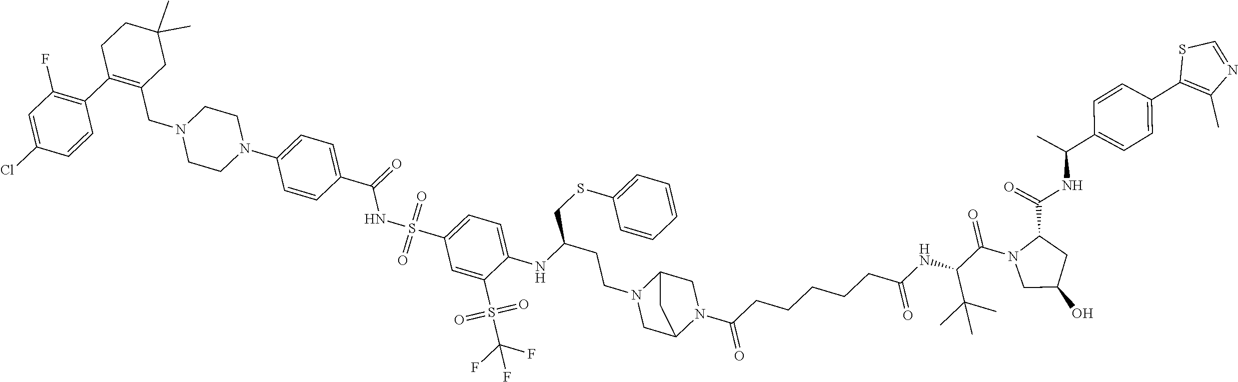 [SMILES: Cc1ncsc1-c1ccc([C@H](C)NC(=O)[C@@H]2C[C@@H](O)CN2C(=O)[C@@H](NC(=O)CCCCCC(=O)N2CC3CC2CN3CC[C@H](CSc2ccccc2)Nc2ccc(S(=O)(=O)NC(=O)c3ccc(N4CCN(CC5=C(c6ccc(Cl)cc6F)CCC(C)(C)C5)CC4)cc3)cc2S(=O)(=O)C(F)(F)F)C(C)(C)C)cc1]

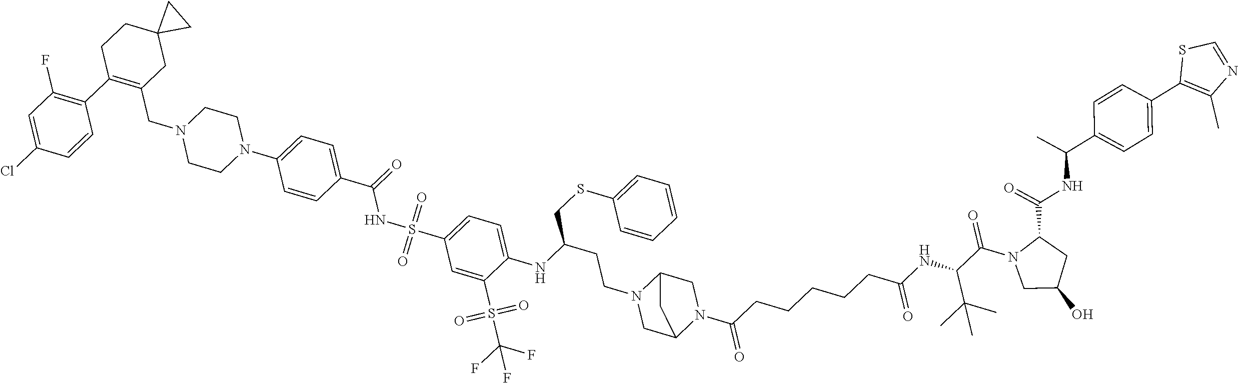 [SMILES: Cc1ncsc1-c1ccc([C@H](C)NC(=O)[C@@H]2C[C@@H](O)CN2C(=O)[C@@H](NC(=O)CCCCCC(=O)N2CC3CC2CN3CC[C@H](CSc2ccccc2)Nc2ccc(S(=O)(=O)NC(=O)c3ccc(N4CCN(CC5=C(c6ccc(Cl)cc6F)CCC6(CC6)C5)CC4)cc3)cc2S(=O)(=O)C(F)(F)F)C(C)(C)C)cc1]

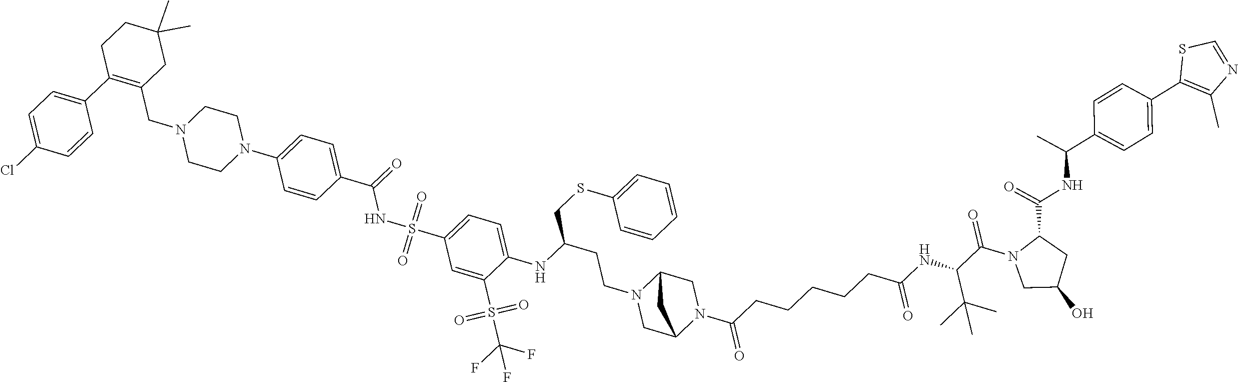 [SMILES: Cc1ncsc1-c1ccc([C@H](C)NC(=O)[C@@H]2C[C@@H](O)CN2C(=O)[C@@H](NC(=O)CCCCCC(=O)N2C[C@@H]3C[C@H]2CN3CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)C(C)(C)C)cc1]

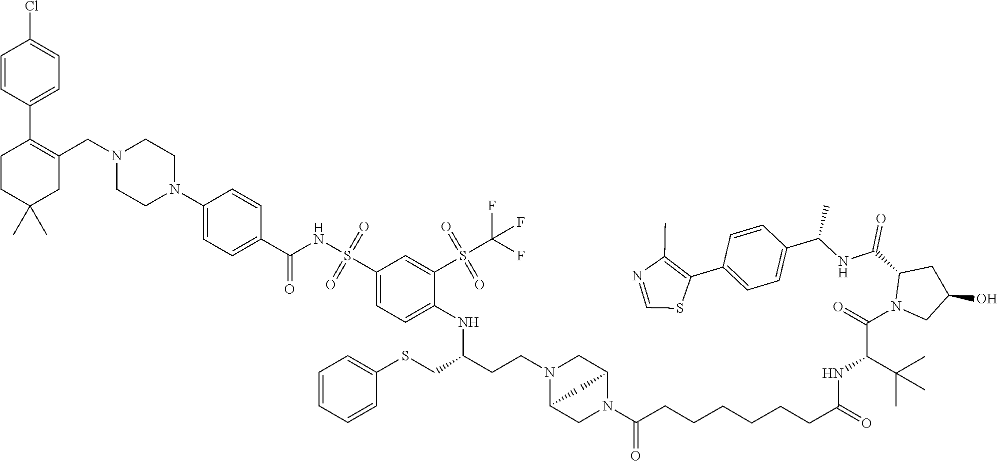 [SMILES: Cc1ncsc1-c1ccc([C@H](C)NC(=O)[C@@H]2C[C@@H](O)CN2C(=O)[C@@H](NC(=O)CCCCCCC(=O)N2C[C@@H]3C[C@H]2CN3CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)C(C)(C)C)cc1]

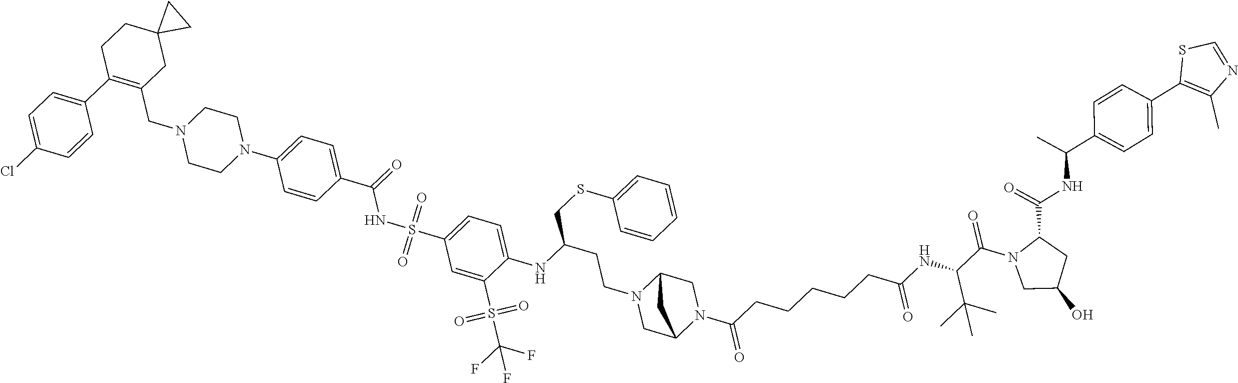 [SMILES: Cc1ncsc1-c1ccc([C@H](C)NC(=O)[C@@H]2C[C@@H](O)CN2C(=O)[C@@H](NC(=O)CCCCCC(=O)N2C[C@@H]3C[C@H]2CN3CC[C@H](CSc2ccccc2)Nc2ccc(S(=O)(=O)NC(=O)c3ccc(N4CCN(CC5=C(c6ccc(Cl)cc6)CCC6(CC6)C5)CC4)cc3)cc2S(=O)(=O)C(F)(F)F)C(C)(C)C)cc1]

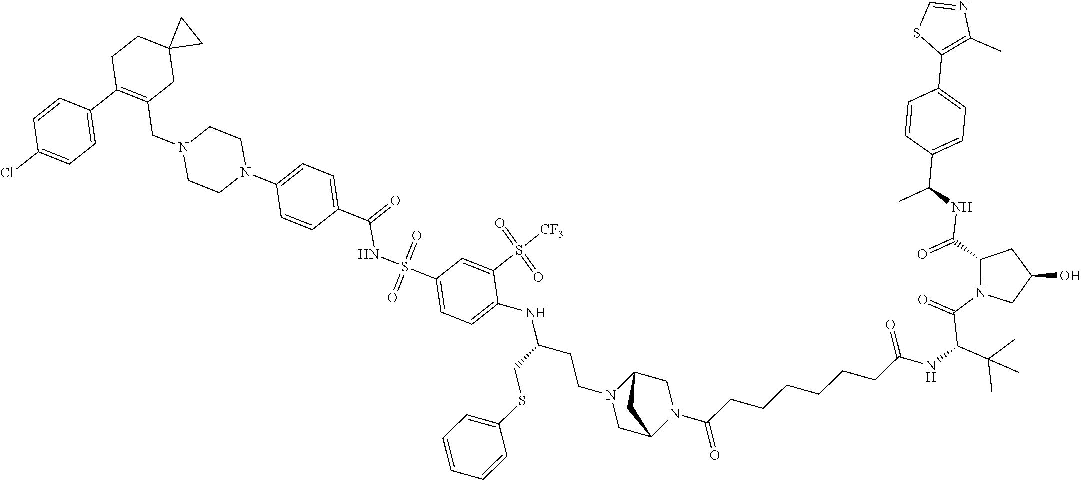 [SMILES: Cc1ncsc1-c1ccc([C@H](C)NC(=O)[C@@H]2C[C@@H](O)CN2C(=O)[C@@H](NC(=O)CCCCCCC(=O)N2C[C@@H]3C[C@H]2CN3CC[C@H](CSc2ccccc2)Nc2ccc(S(=O)(=O)NC(=O)c3ccc(N4CCN(CC5=C(c6ccc(Cl)cc6)CCC6(CC6)C5)CC4)cc3)cc2S(=O)(=O)C(F)(F)F)C(C)(C)C)cc1]

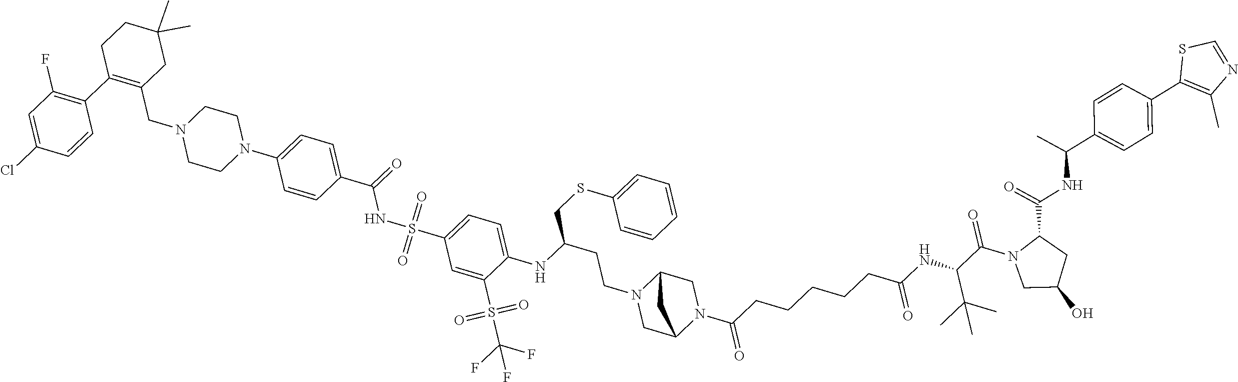 [SMILES: Cc1ncsc1-c1ccc([C@H](C)NC(=O)[C@@H]2C[C@@H](O)CN2C(=O)[C@@H](NC(=O)CCCCCC(=O)N2C[C@@H]3C[C@H]2CN3CC[C@H](CSc2ccccc2)Nc2ccc(S(=O)(=O)NC(=O)c3ccc(N4CCN(CC5=C(c6ccc(Cl)cc6F)CCC(C)(C)C5)CC4)cc3)cc2S(=O)(=O)C(F)(F)F)C(C)(C)C)cc1]

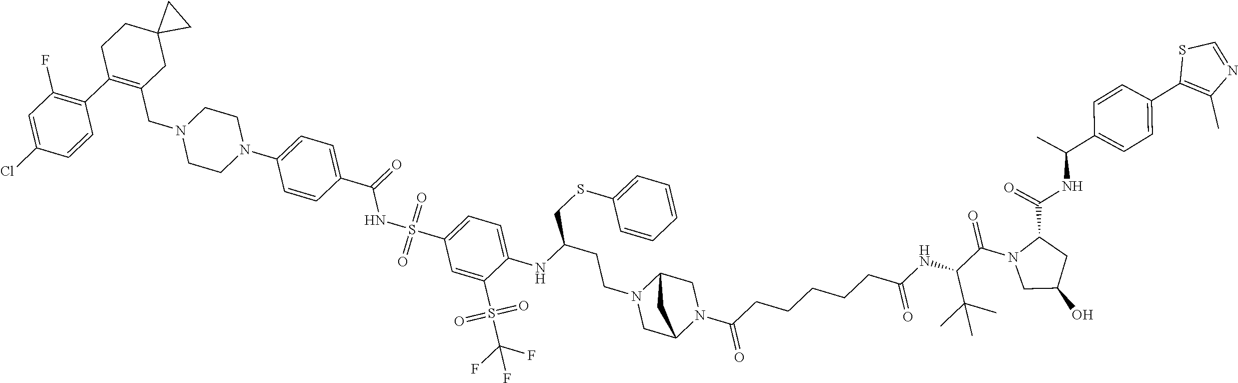 [SMILES: Cc1ncsc1-c1ccc([C@H](C)NC(=O)[C@@H]2C[C@@H](O)CN2C(=O)[C@@H](NC(=O)CCCCCC(=O)N2C[C@@H]3C[C@H]2CN3CC[C@H](CSc2ccccc2)Nc2ccc(S(=O)(=O)NC(=O)c3ccc(N4CCN(CC5=C(c6ccc(Cl)cc6F)CCC6(CC6)C5)CC4)cc3)cc2S(=O)(=O)C(F)(F)F)C(C)(C)C)cc1]

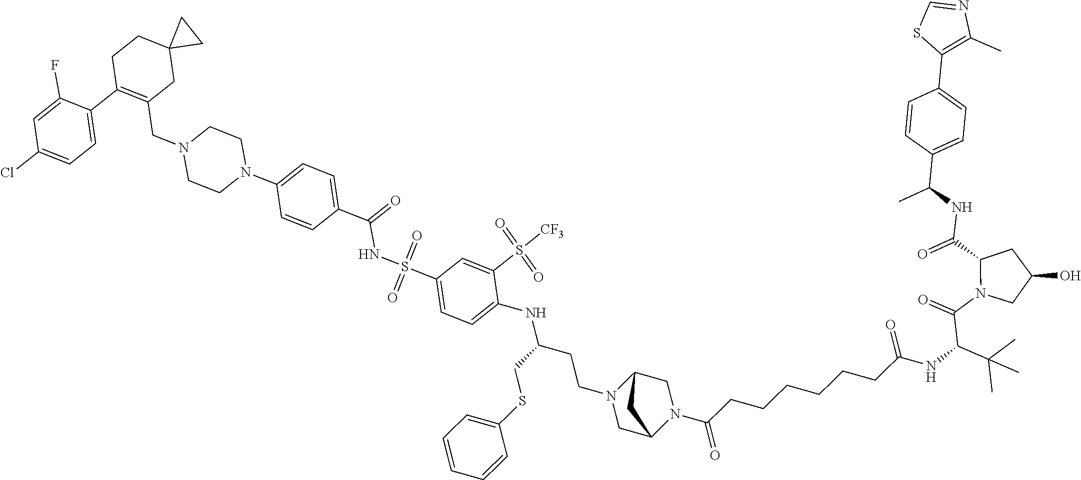 [SMILES: Cc1ncsc1-c1ccc([C@H](C)NC(=O)[C@@H]2C[C@@H](O)CN2C(=O)[C@@H](NC(=O)CCCCCCC(=O)N2C[C@@H]3C[C@H]2CN3CC[C@H](CSc2ccccc2)Nc2ccc(S(=O)(=O)NC(=O)c3ccc(N4CCN(CC5=C(c6ccc(Cl)cc6F)CCC6(CC6)C5)CC4)cc3)cc2S(=O)(=O)C(F)(F)F)C(C)(C)C)cc1]

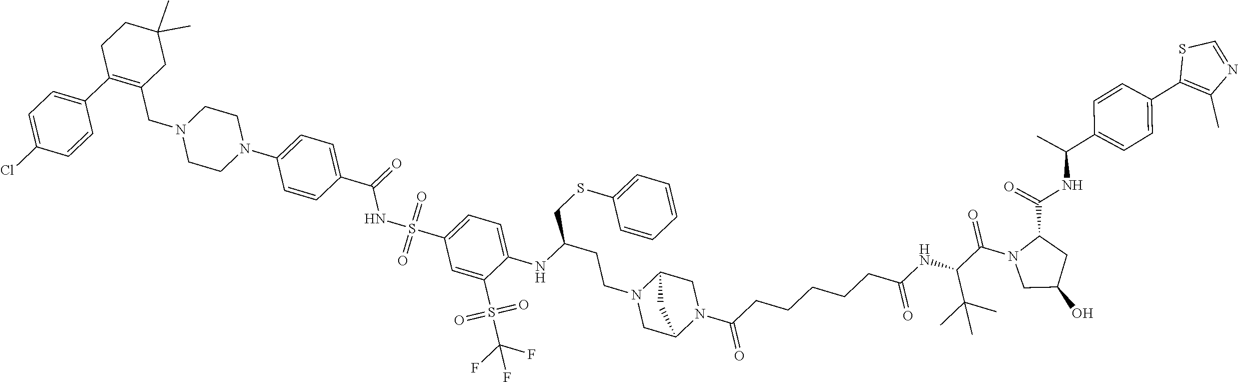 [SMILES: Cc1ncsc1-c1ccc([C@H](C)NC(=O)[C@@H]2C[C@@H](O)CN2C(=O)[C@@H](NC(=O)CCCCCC(=O)N2C[C@H]3C[C@@H]2CN3CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)C(C)(C)C)cc1]

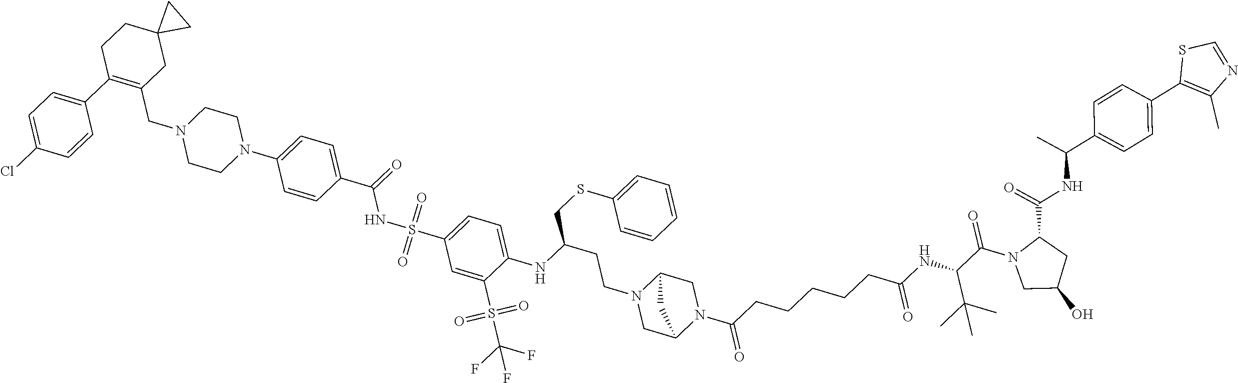 [SMILES: Cc1ncsc1-c1ccc([C@H](C)NC(=O)[C@@H]2C[C@@H](O)CN2C(=O)[C@@H](NC(=O)CCCCCC(=O)N2C[C@H]3C[C@@H]2CN3CC[C@H](CSc2ccccc2)Nc2ccc(S(=O)(=O)NC(=O)c3ccc(N4CCN(CC5=C(c6ccc(Cl)cc6)CCC6(CC6)C5)CC4)cc3)cc2S(=O)(=O)C(F)(F)F)C(C)(C)C)cc1]

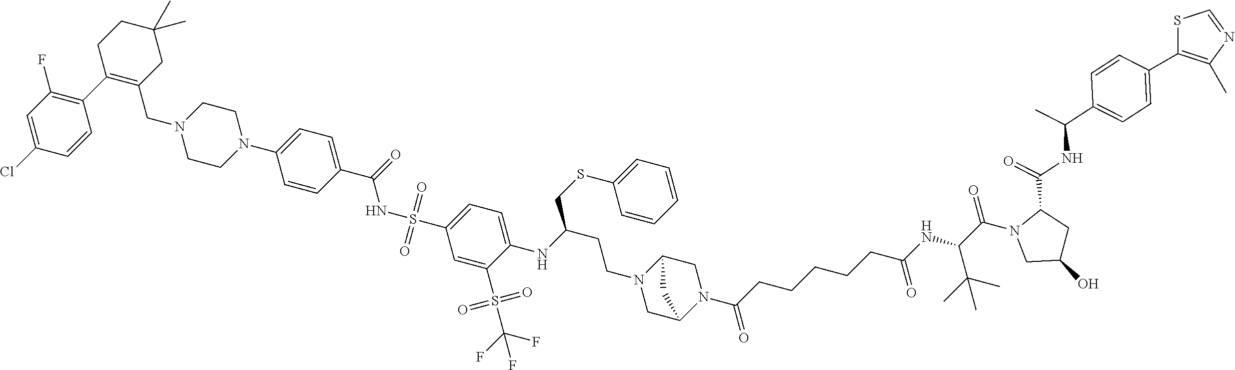 [SMILES: Cc1ncsc1-c1ccc([C@H](C)NC(=O)[C@@H]2C[C@@H](O)CN2C(=O)[C@@H](NC(=O)CCCCCC(=O)N2C[C@H]3C[C@@H]2CN3CC[C@H](CSc2ccccc2)Nc2ccc(S(=O)(=O)NC(=O)c3ccc(N4CCN(CC5=C(c6ccc(Cl)cc6F)CCC(C)(C)C5)CC4)cc3)cc2S(=O)(=O)C(F)(F)F)C(C)(C)C)cc1]

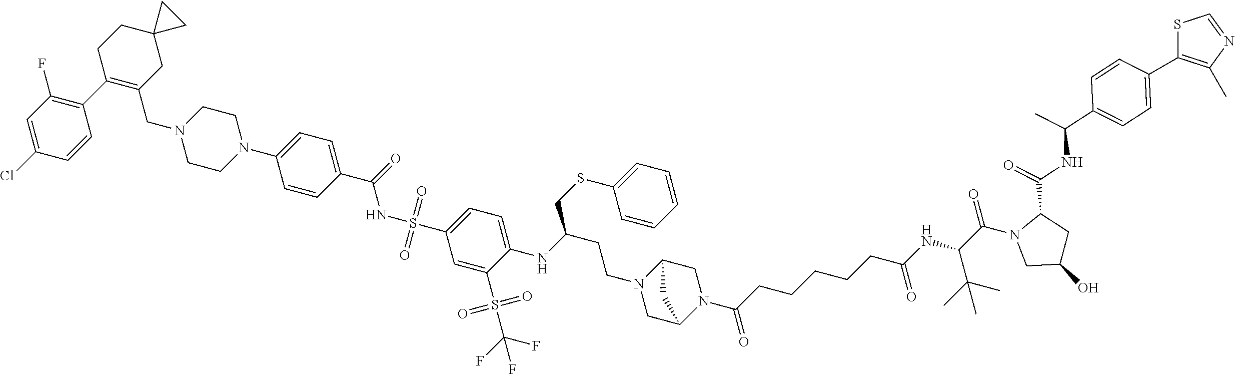 [SMILES: Cc1ncsc1-c1ccc([C@H](C)NC(=O)[C@@H]2C[C@@H](O)CN2C(=O)[C@@H](NC(=O)CCCCCC(=O)N2C[C@H]3C[C@@H]2CN3CC[C@H](CSc2ccccc2)Nc2ccc(S(=O)(=O)NC(=O)c3ccc(N4CCN(CC5=C(c6ccc(Cl)cc6F)CCC6(CC6)C5)CC4)cc3)cc2S(=O)(=O)C(F)(F)F)C(C)(C)C)cc1]

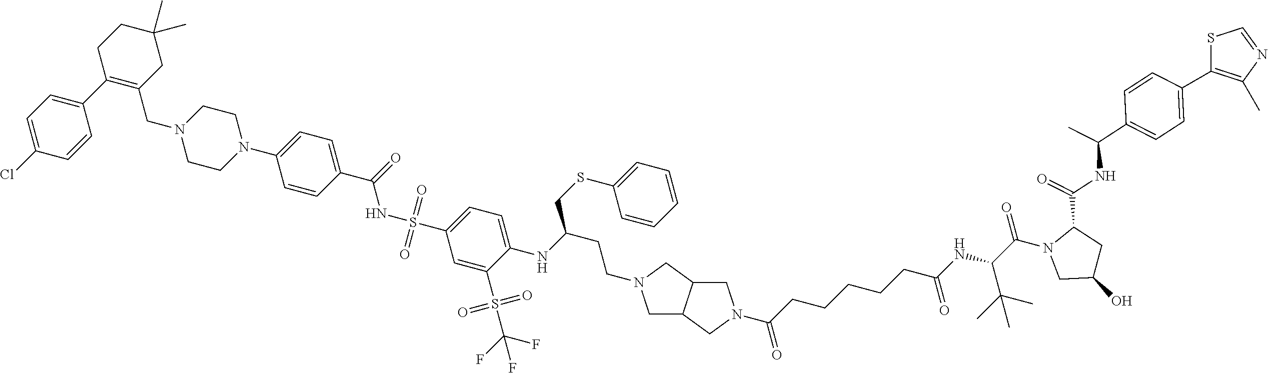 [SMILES: Cc1ncsc1-c1ccc([C@H](C)NC(=O)[C@@H]2C[C@@H](O)CN2C(=O)[C@@H](NC(=O)CCCCCC(=O)N2CC3CN(CC[C@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)CC3C2)C(C)(C)C)cc1]

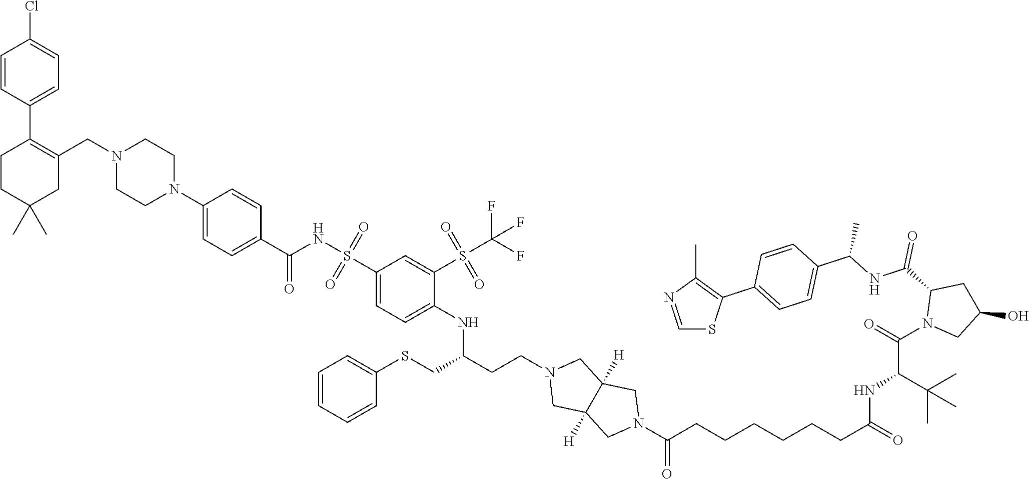 [SMILES: Cc1ncsc1-c1ccc([C@H](C)NC(=O)[C@@H]2C[C@@H](O)CN2C(=O)[C@@H](NC(=O)CCCCCCC(=O)N2C[C@H]3CN(CC[C@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)C[C@H]3C2)C(C)(C)C)cc1]

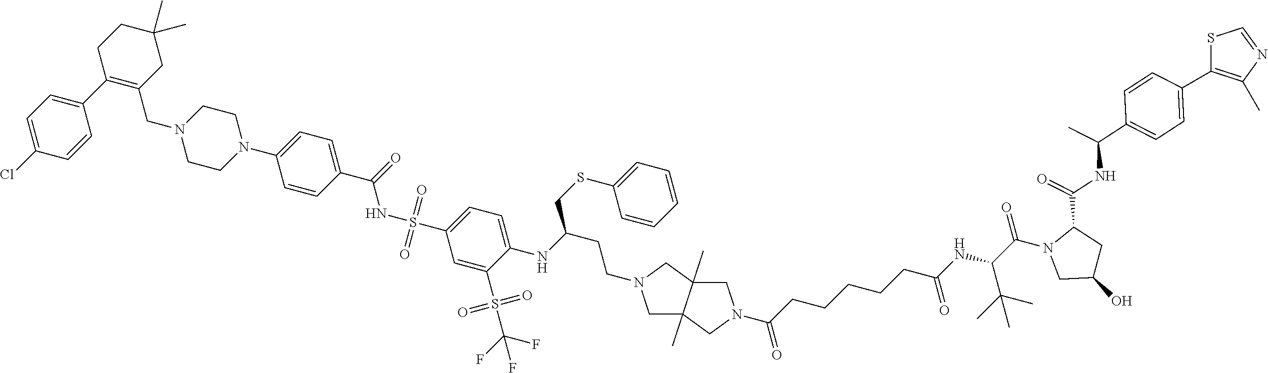 [SMILES: Cc1ncsc1-c1ccc([C@H](C)NC(=O)[C@@H]2C[C@@H](O)CN2C(=O)[C@@H](NC(=O)CCCCCC(=O)N2CC3(C)CN(CC[C@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)CC3(C)C2)C(C)(C)C)cc1]

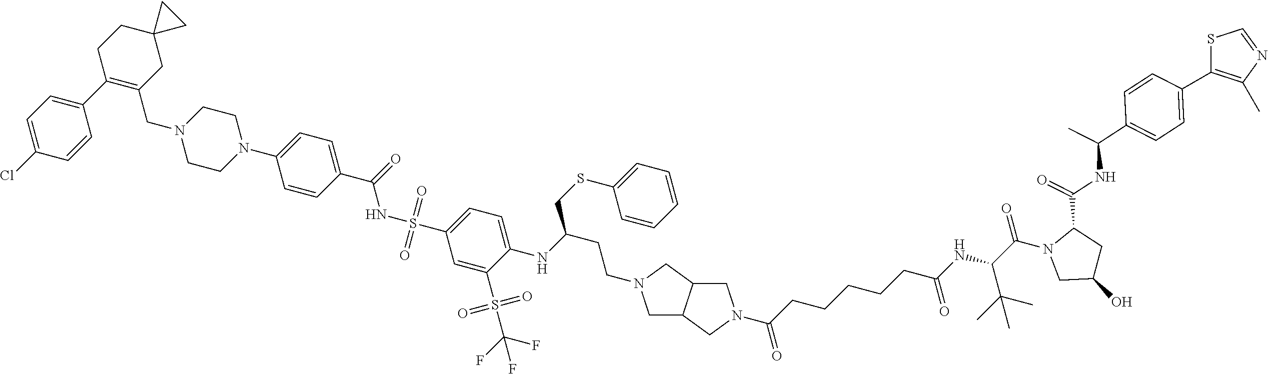 [SMILES: Cc1ncsc1-c1ccc([C@H](C)NC(=O)[C@@H]2C[C@@H](O)CN2C(=O)[C@@H](NC(=O)CCCCCC(=O)N2CC3CN(CC[C@H](CSc4ccccc4)Nc4ccc(S(=O)(=O)NC(=O)c5ccc(N6CCN(CC7=C(c8ccc(Cl)cc8)CCC8(CC8)C7)CC6)cc5)cc4S(=O)(=O)C(F)(F)F)CC3C2)C(C)(C)C)cc1]

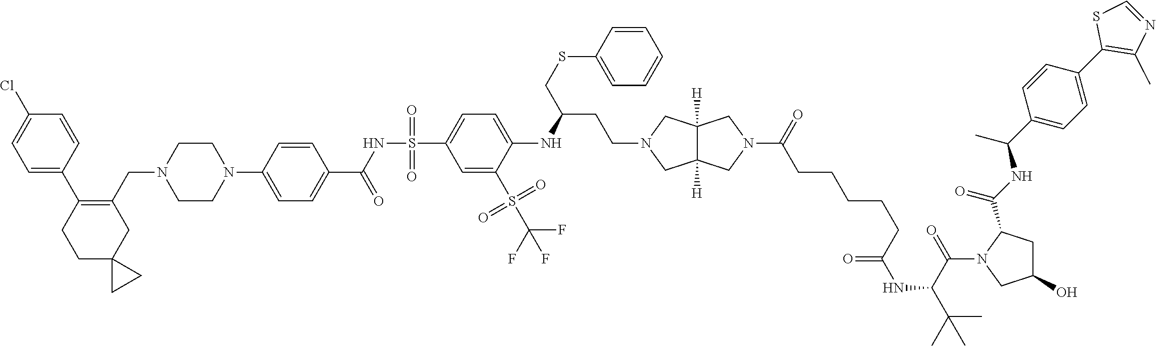 [SMILES: Cc1ncsc1-c1ccc([C@H](C)NC(=O)[C@@H]2C[C@@H](O)CN2C(=O)[C@@H](NC(=O)CCCCCC(=O)N2C[C@H]3CN(CC[C@H](CSc4ccccc4)Nc4ccc(S(=O)(=O)NC(=O)c5ccc(N6CCN(CC7=C(c8ccc(Cl)cc8)CCC8(CC8)C7)CC6)cc5)cc4S(=O)(=O)C(F)(F)F)C[C@H]3C2)C(C)(C)C)cc1]

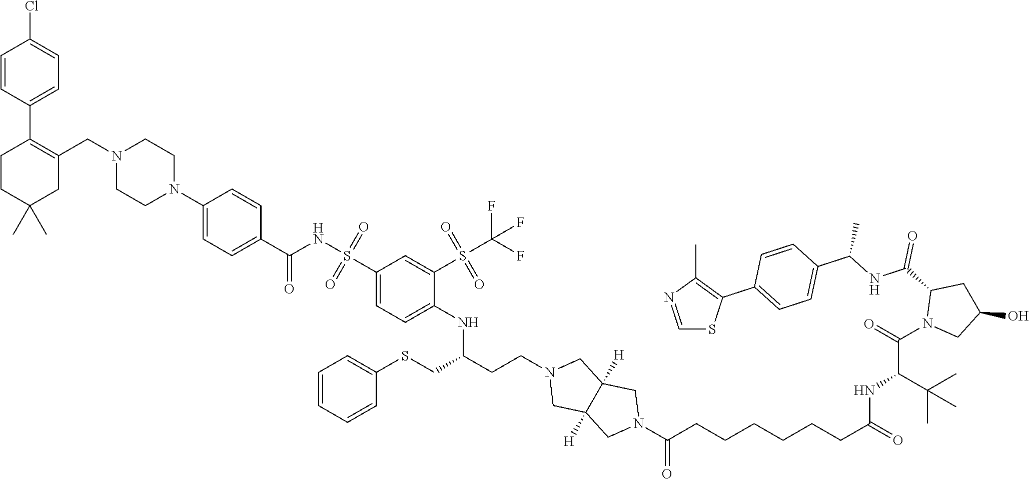 [SMILES: Cc1ncsc1-c1ccc([C@H](C)NC(=O)[C@@H]2C[C@@H](O)CN2C(=O)[C@@H](NC(=O)CCCCCCC(=O)N2C[C@H]3CN(CC[C@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)C[C@H]3C2)C(C)(C)C)cc1]

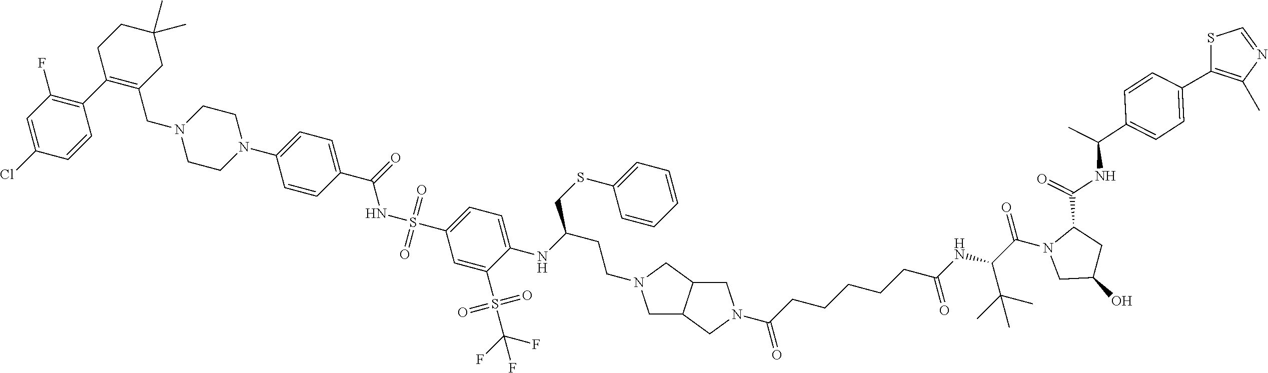 [SMILES: Cc1ncsc1-c1ccc([C@H](C)NC(=O)[C@@H]2C[C@@H](O)CN2C(=O)[C@@H](NC(=O)CCCCCC(=O)N2CC3CN(CC[C@H](CSc4ccccc4)Nc4ccc(S(=O)(=O)NC(=O)c5ccc(N6CCN(CC7=C(c8ccc(Cl)cc8F)CCC(C)(C)C7)CC6)cc5)cc4S(=O)(=O)C(F)(F)F)CC3C2)C(C)(C)C)cc1]

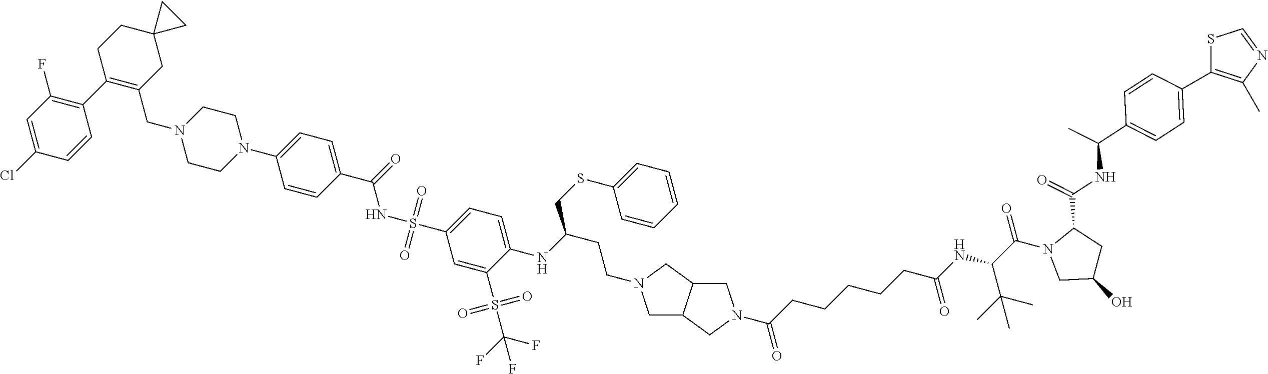 [SMILES: Cc1ncsc1-c1ccc([C@H](C)NC(=O)[C@@H]2C[C@@H](O)CN2C(=O)[C@@H](NC(=O)CCCCCC(=O)N2CC3CN(CC[C@H](CSc4ccccc4)Nc4ccc(S(=O)(=O)NC(=O)c5ccc(N6CCN(CC7=C(c8ccc(Cl)cc8F)CCC8(CC8)C7)CC6)cc5)cc4S(=O)(=O)C(F)(F)F)CC3C2)C(C)(C)C)cc1]

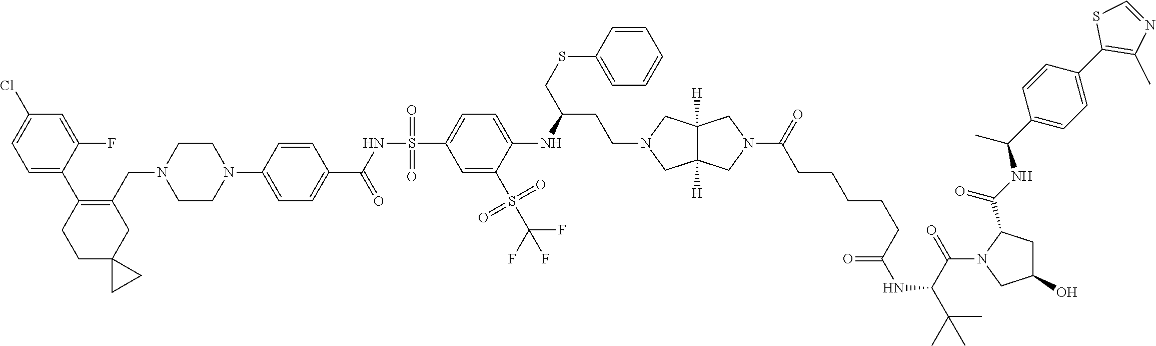 [SMILES: Cc1ncsc1-c1ccc([C@H](C)NC(=O)[C@@H]2C[C@@H](O)CN2C(=O)[C@@H](NC(=O)CCCCCC(=O)N2C[C@H]3CN(CC[C@H](CSc4ccccc4)Nc4ccc(S(=O)(=O)NC(=O)c5ccc(N6CCN(CC7=C(c8ccc(Cl)cc8F)CCC8(CC8)C7)CC6)cc5)cc4S(=O)(=O)C(F)(F)F)C[C@H]3C2)C(C)(C)C)cc1]

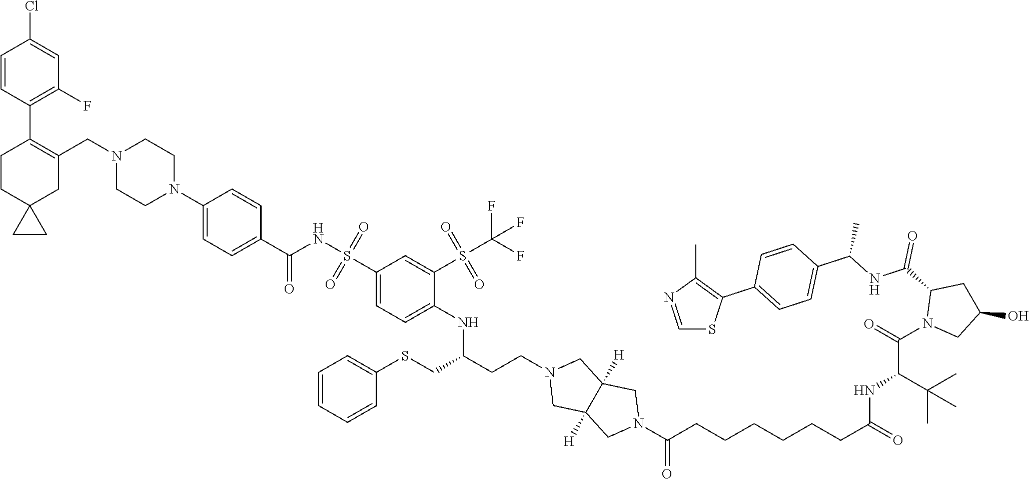 [SMILES: Cc1ncsc1-c1ccc([C@H](C)NC(=O)[C@@H]2C[C@@H](O)CN2C(=O)[C@@H](NC(=O)CCCCCCC(=O)N2C[C@H]3CN(CC[C@H](CSc4ccccc4)Nc4ccc(S(=O)(=O)NC(=O)c5ccc(N6CCN(CC7=C(c8ccc(Cl)cc8F)CCC8(CC8)C7)CC6)cc5)cc4S(=O)(=O)C(F)(F)F)C[C@H]3C2)C(C)(C)C)cc1]

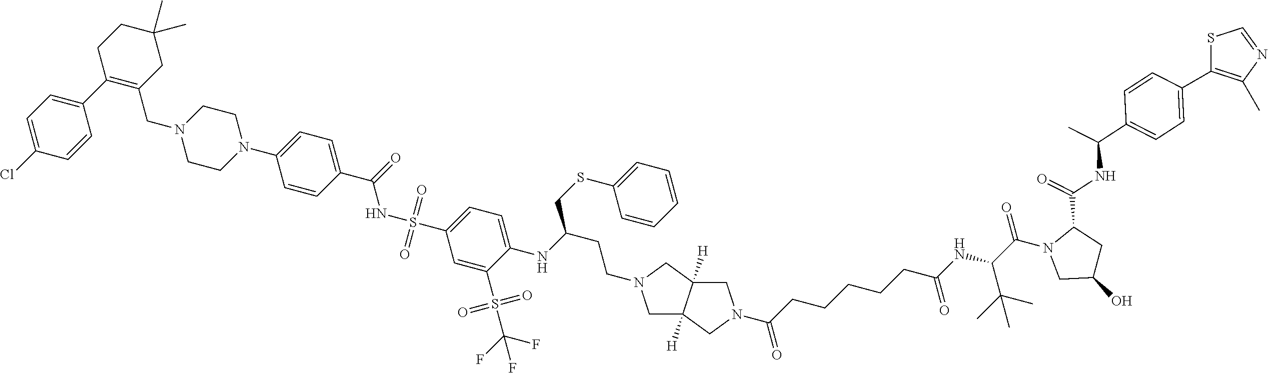 [SMILES: Cc1ncsc1-c1ccc([C@H](C)NC(=O)[C@@H]2C[C@@H](O)CN2C(=O)[C@@H](NC(=O)CCCCCC(=O)N2C[C@H]3CN(CC[C@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)C[C@H]3C2)C(C)(C)C)cc1]

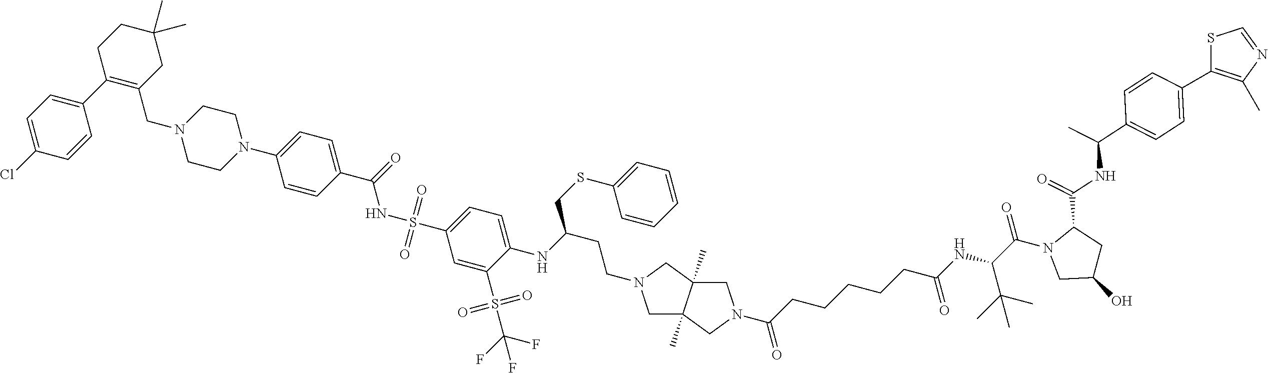 [SMILES: Cc1ncsc1-c1ccc([C@H](C)NC(=O)[C@@H]2C[C@@H](O)CN2C(=O)[C@@H](NC(=O)CCCCCC(=O)N2C[C@]3(C)CN(CC[C@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)C[C@]3(C)C2)C(C)(C)C)cc1]

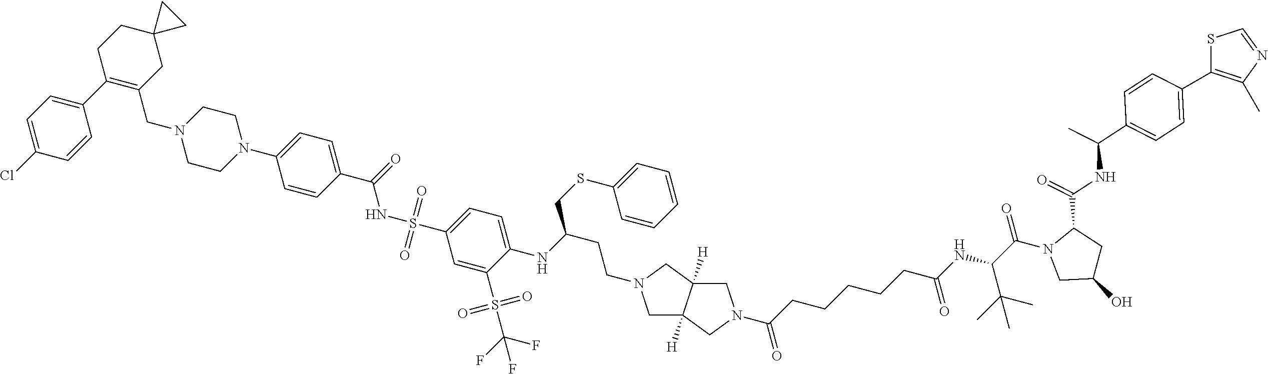 [SMILES: Cc1ncsc1-c1ccc([C@H](C)NC(=O)[C@@H]2C[C@@H](O)CN2C(=O)[C@@H](NC(=O)CCCCCC(=O)N2C[C@H]3CN(CC[C@H](CSc4ccccc4)Nc4ccc(S(=O)(=O)NC(=O)c5ccc(N6CCN(CC7=C(c8ccc(Cl)cc8)CCC8(CC8)C7)CC6)cc5)cc4S(=O)(=O)C(F)(F)F)C[C@H]3C2)C(C)(C)C)cc1]

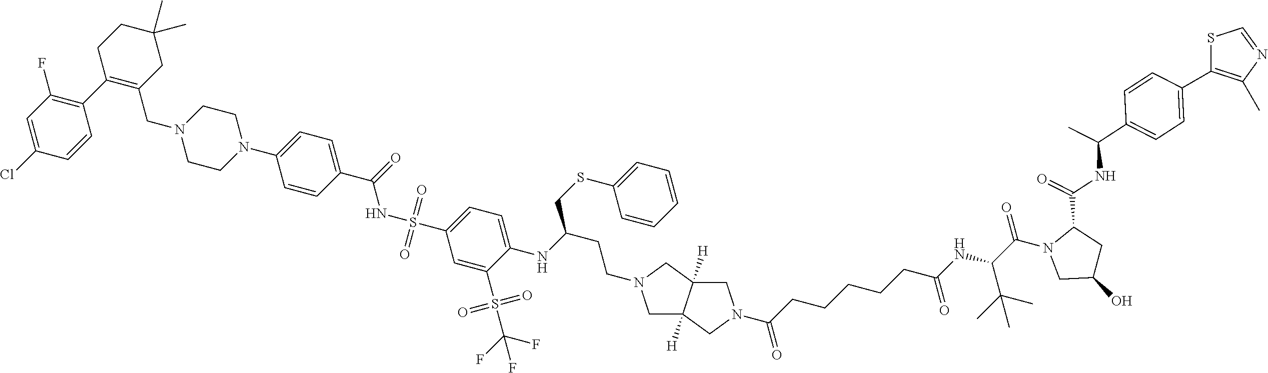 [SMILES: Cc1ncsc1-c1ccc([C@H](C)NC(=O)[C@@H]2C[C@@H](O)CN2C(=O)[C@@H](NC(=O)CCCCCC(=O)N2C[C@H]3CN(CC[C@H](CSc4ccccc4)Nc4ccc(S(=O)(=O)NC(=O)c5ccc(N6CCN(CC7=C(c8ccc(Cl)cc8F)CCC(C)(C)C7)CC6)cc5)cc4S(=O)(=O)C(F)(F)F)C[C@H]3C2)C(C)(C)C)cc1]

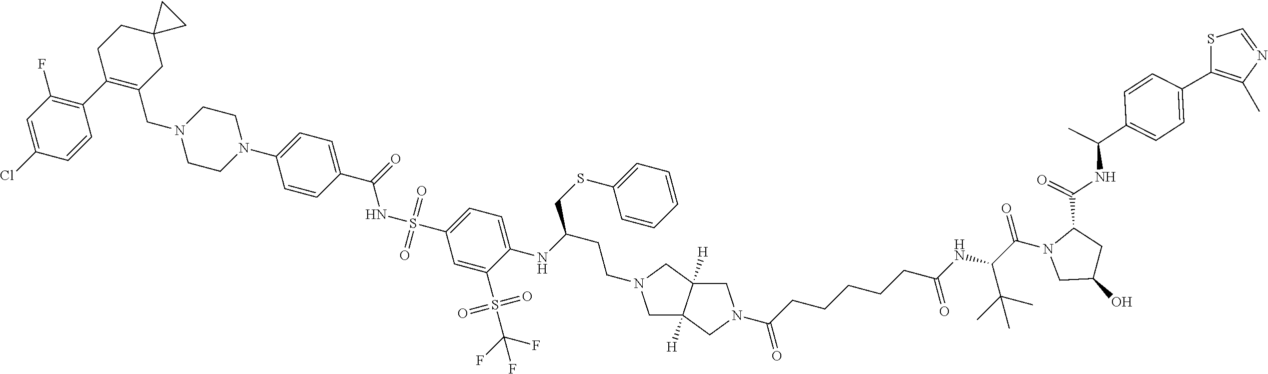 [SMILES: Cc1ncsc1-c1ccc([C@H](C)NC(=O)[C@@H]2C[C@@H](O)CN2C(=O)[C@@H](NC(=O)CCCCCC(=O)N2C[C@H]3CN(CC[C@H](CSc4ccccc4)Nc4ccc(S(=O)(=O)NC(=O)c5ccc(N6CCN(CC7=C(c8ccc(Cl)cc8F)CCC8(CC8)C7)CC6)cc5)cc4S(=O)(=O)C(F)(F)F)C[C@H]3C2)C(C)(C)C)cc1]

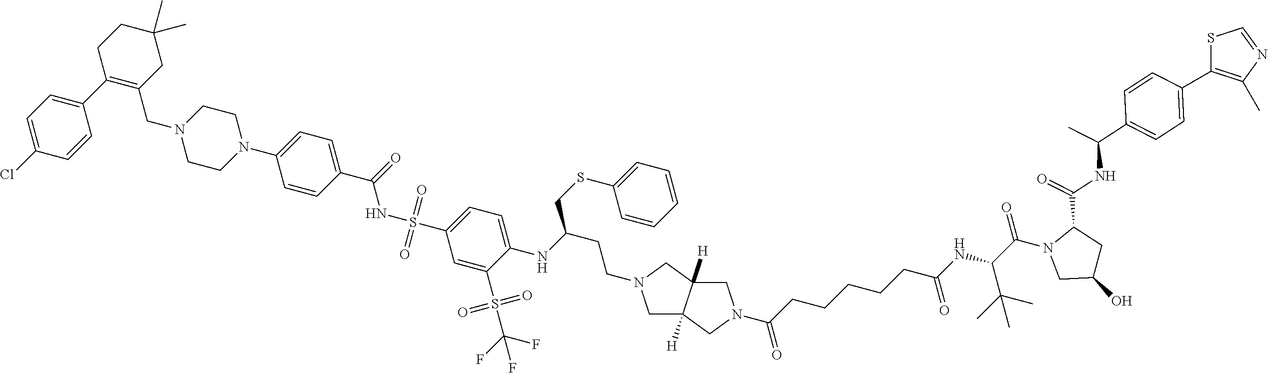 [SMILES: Cc1ncsc1-c1ccc([C@H](C)NC(=O)[C@@H]2C[C@@H](O)CN2C(=O)[C@@H](NC(=O)CCCCCC(=O)N2C[C@H]3CN(CC[C@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)C[C@@H]3C2)C(C)(C)C)cc1]

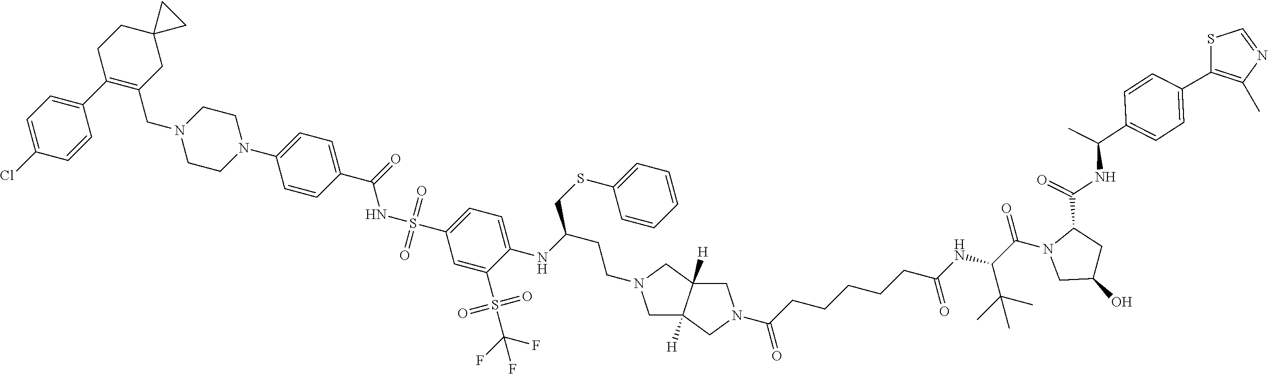 [SMILES: Cc1ncsc1-c1ccc([C@H](C)NC(=O)[C@@H]2C[C@@H](O)CN2C(=O)[C@@H](NC(=O)CCCCCC(=O)N2C[C@H]3CN(CC[C@H](CSc4ccccc4)Nc4ccc(S(=O)(=O)NC(=O)c5ccc(N6CCN(CC7=C(c8ccc(Cl)cc8)CCC8(CC8)C7)CC6)cc5)cc4S(=O)(=O)C(F)(F)F)C[C@@H]3C2)C(C)(C)C)cc1]

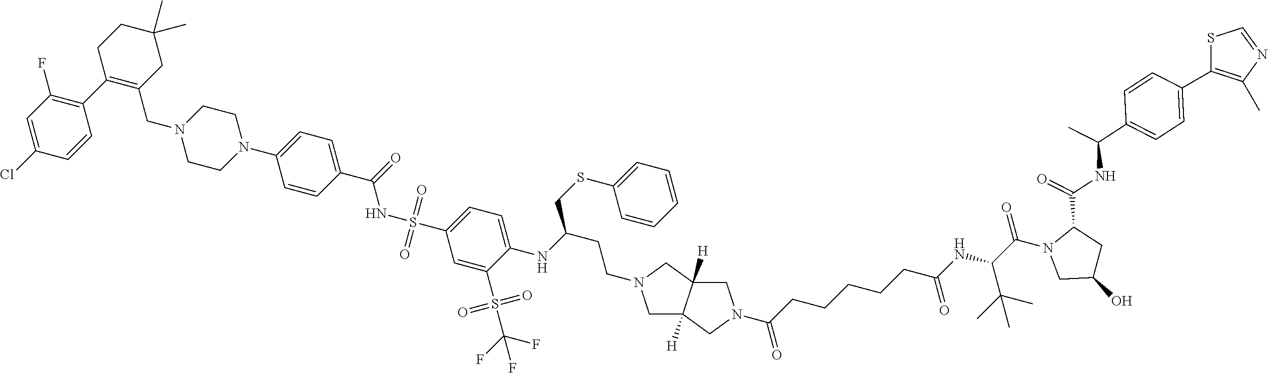 [SMILES: Cc1ncsc1-c1ccc([C@H](C)NC(=O)[C@@H]2C[C@@H](O)CN2C(=O)[C@@H](NC(=O)CCCCCC(=O)N2C[C@H]3CN(CC[C@H](CSc4ccccc4)Nc4ccc(S(=O)(=O)NC(=O)c5ccc(N6CCN(CC7=C(c8ccc(Cl)cc8F)CCC(C)(C)C7)CC6)cc5)cc4S(=O)(=O)C(F)(F)F)C[C@@H]3C2)C(C)(C)C)cc1]

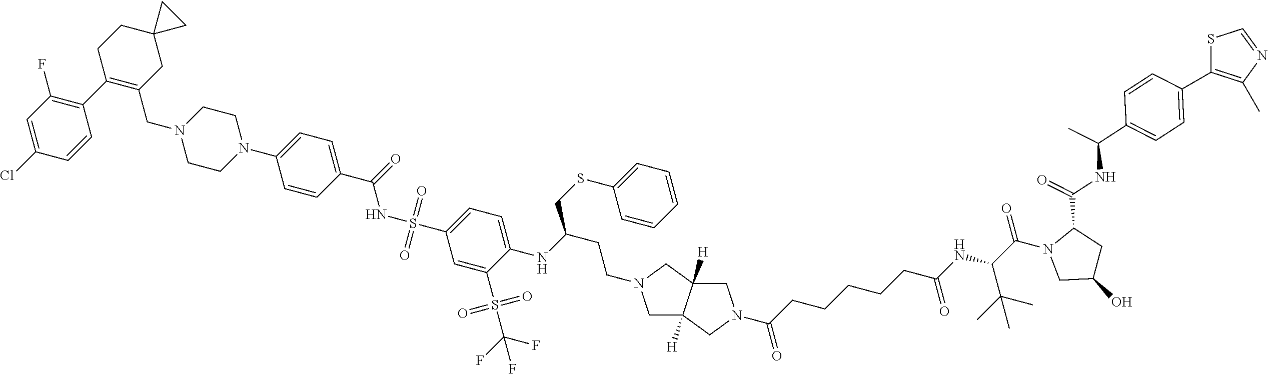 [SMILES: Cc1ncsc1-c1ccc([C@H](C)NC(=O)[C@@H]2C[C@@H](O)CN2C(=O)[C@@H](NC(=O)CCCCCC(=O)N2C[C@H]3CN(CC[C@H](CSc4ccccc4)Nc4ccc(S(=O)(=O)NC(=O)c5ccc(N6CCN(CC7=C(c8ccc(Cl)cc8F)CCC8(CC8)C7)CC6)cc5)cc4S(=O)(=O)C(F)(F)F)C[C@@H]3C2)C(C)(C)C)cc1]

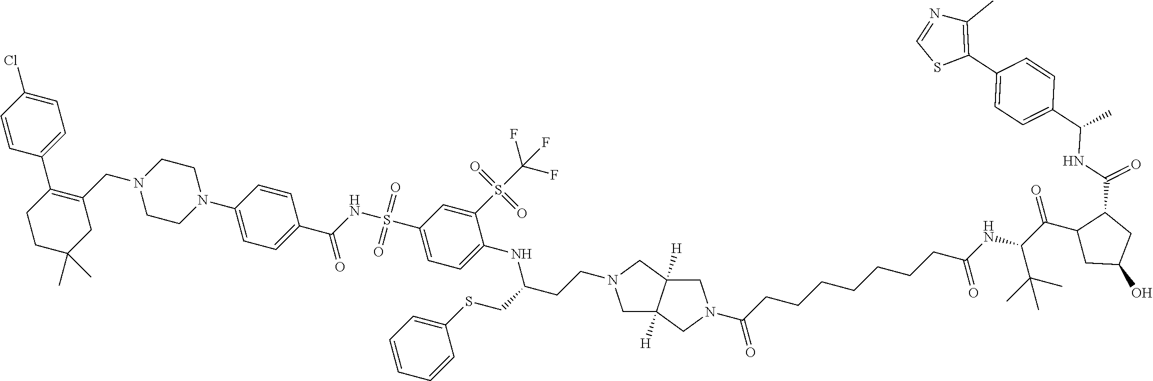 [SMILES: Cc1ncsc1-c1ccc([C@H](C)NC(=O)[C@@H]2C[C@@H](O)CC2C(=O)[C@@H](NC(=O)CCCCCCCC(=O)N2C[C@H]3CN(CC[C@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)C[C@H]3C2)C(C)(C)C)cc1]

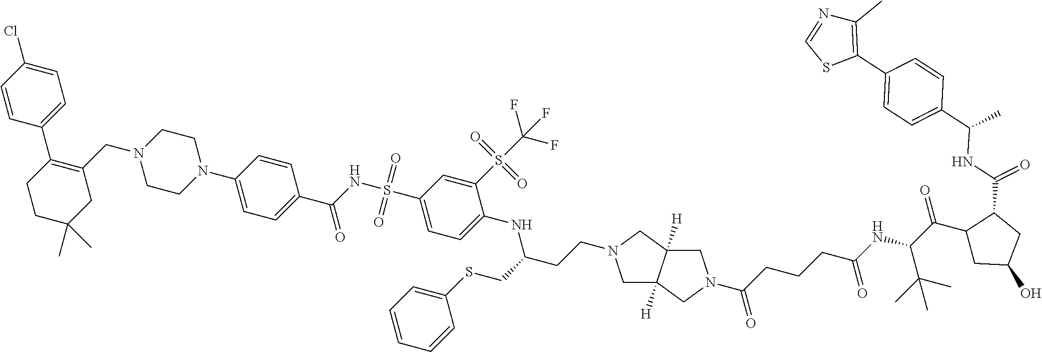 [SMILES: Cc1ncsc1-c1ccc([C@H](C)NC(=O)[C@@H]2C[C@@H](O)CC2C(=O)[C@@H](NC(=O)CCCC(=O)N2C[C@H]3CN(CC[C@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)C[C@H]3C2)C(C)(C)C)cc1]

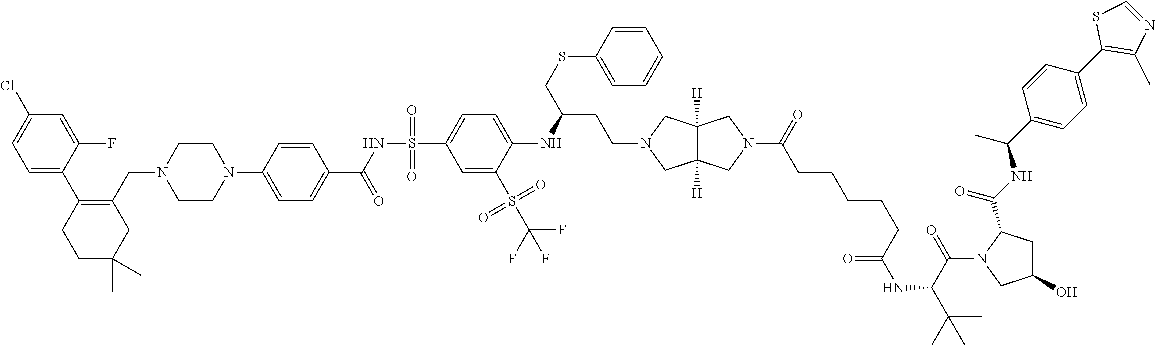 [SMILES: Cc1ncsc1-c1ccc([C@H](C)NC(=O)[C@@H]2C[C@@H](O)CN2C(=O)[C@@H](NC(=O)CCCCCC(=O)N2C[C@H]3CN(CC[C@H](CSc4ccccc4)Nc4ccc(S(=O)(=O)NC(=O)c5ccc(N6CCN(CC7=C(c8ccc(Cl)cc8F)CCC(C)(C)C7)CC6)cc5)cc4S(=O)(=O)C(F)(F)F)C[C@H]3C2)C(C)(C)C)cc1]

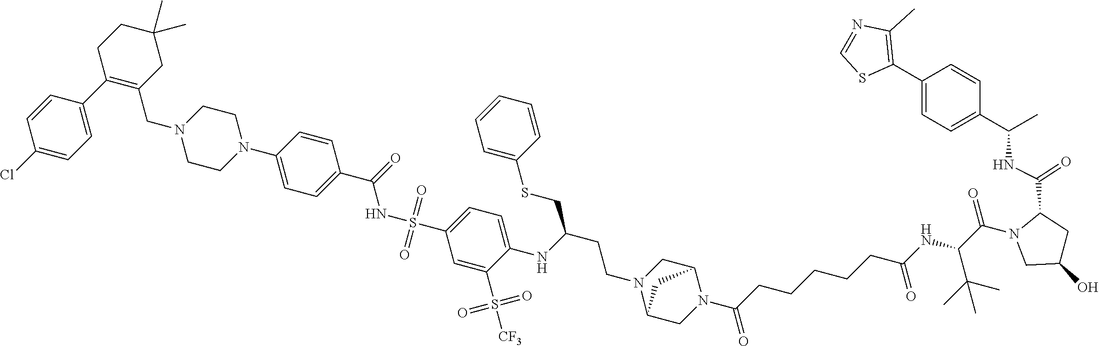 [SMILES: Cc1ncsc1-c1ccc([C@H](C)NC(=O)[C@@H]2C[C@@H](O)CN2C(=O)[C@@H](NC(=O)CCCCCC(=O)N2C[C@@H]3C[C@H]2CN3CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)C(C)(C)C)cc1]

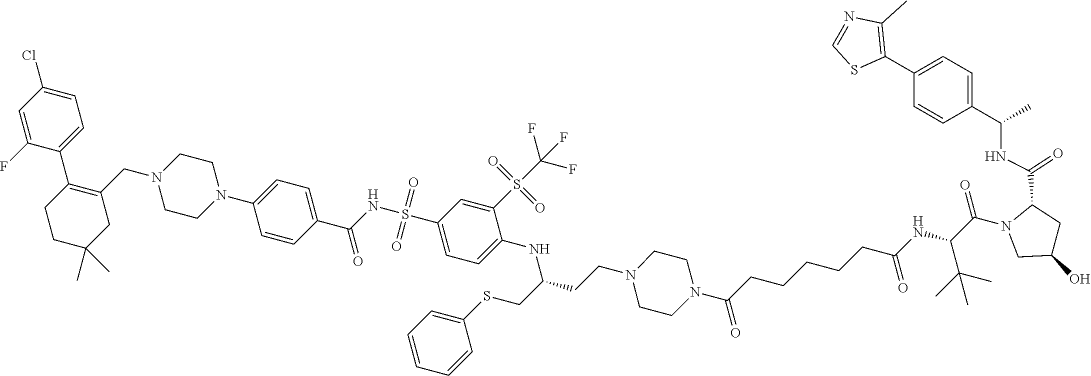 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7F)CCC(C)(C)C6)CC5)cc4)cc3S(=O)(=O)C(F)(F)F)CC2)C(C)(C)C)cc1]

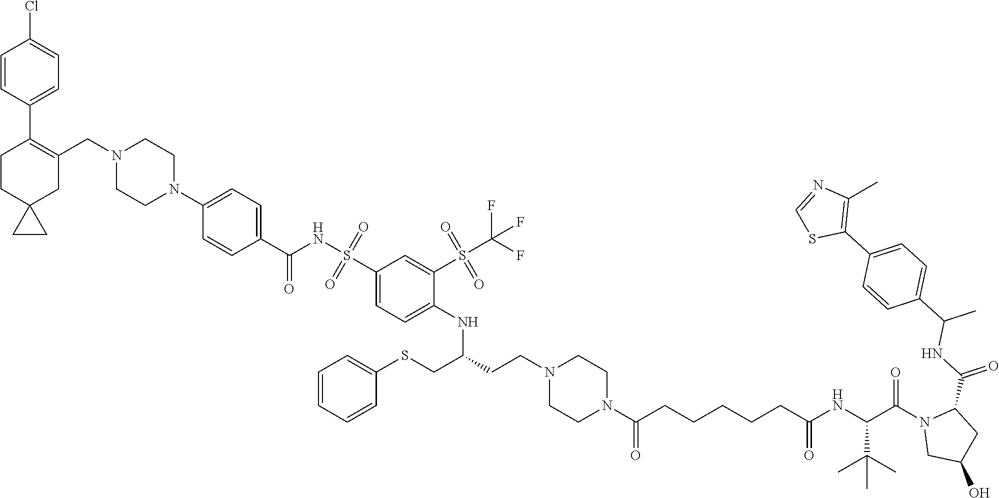 [SMILES: Cc1ncsc1-c1ccc(C(C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC7(CC7)C6)CC5)cc4)cc3S(=O)(=O)C(F)(F)F)CC2)C(C)(C)C)cc1]

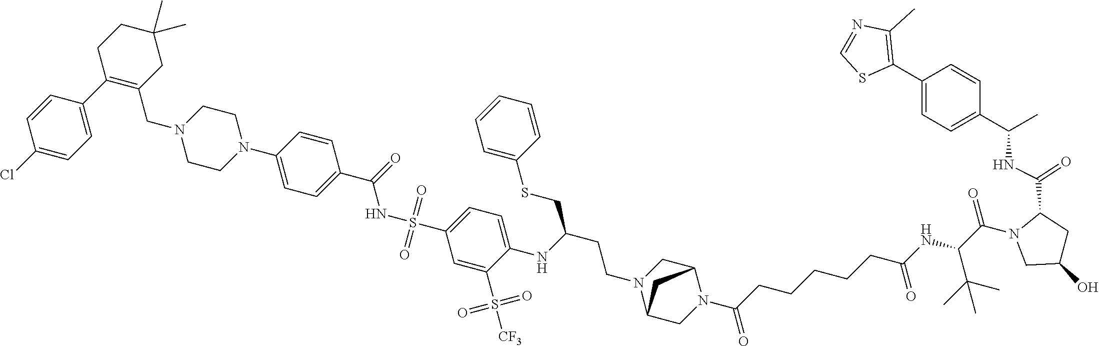 [SMILES: Cc1ncsc1-c1ccc([C@H](C)NC(=O)[C@@H]2C[C@@H](O)CN2C(=O)[C@@H](NC(=O)CCCCCC(=O)N2C[C@H]3C[C@@H]2CN3CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)C(C)(C)C)cc1]

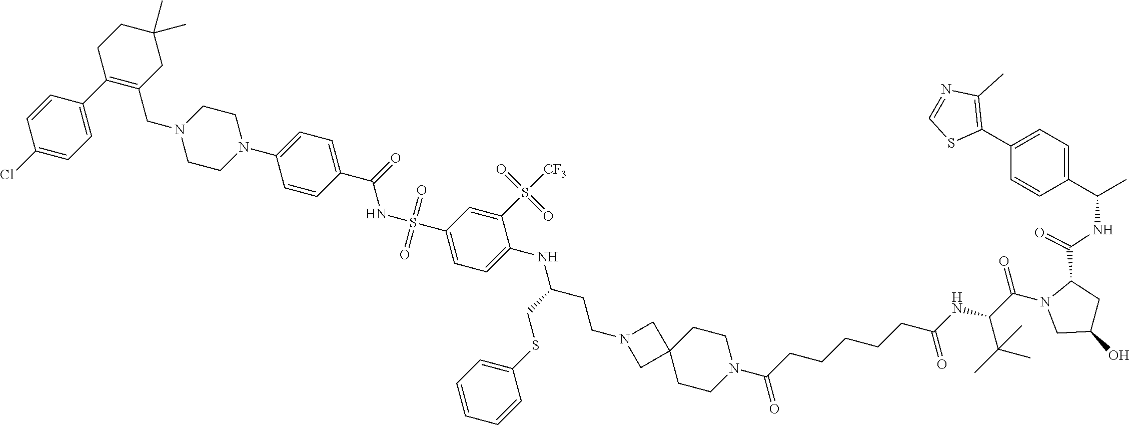 [SMILES: Cc1ncsc1-c1ccc([C@H](C)NC(=O)[C@@H]2C[C@@H](O)CN2C(=O)[C@@H](NC(=O)CCCCCC(=O)N2CCC3(CC2)CN(CC[C@H](CSc2ccccc2)Nc2ccc(S(=O)(=O)NC(=O)c4ccc(N5CCN(CC6=C(c7ccc(Cl)cc7)CCC(C)(C)C6)CC5)cc4)cc2S(=O)(=O)C(F)(F)F)C3)C(C)(C)C)cc1]

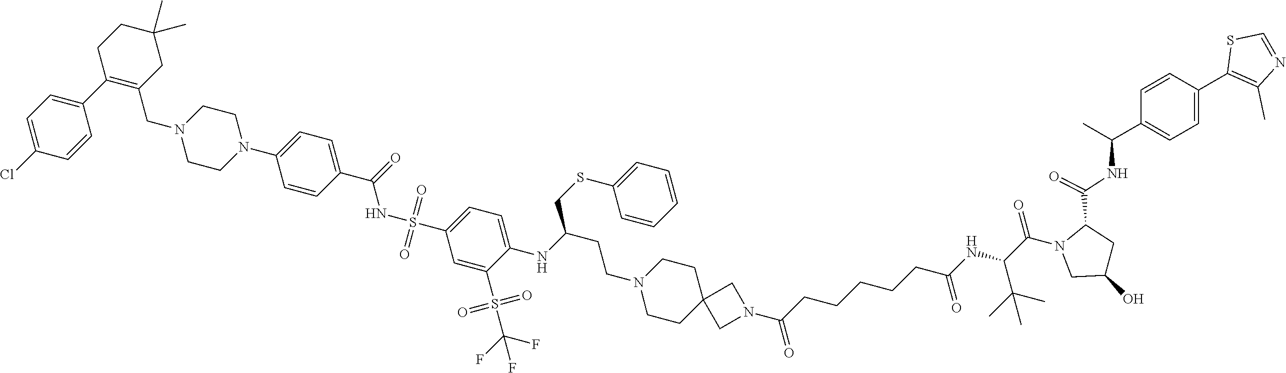 [SMILES: Cc1ncsc1-c1ccc([C@H](C)NC(=O)[C@@H]2C[C@@H](O)CN2C(=O)[C@@H](NC(=O)CCCCCC(=O)N2CC3(CCN(CC[C@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)CC3)C2)C(C)(C)C)cc1]

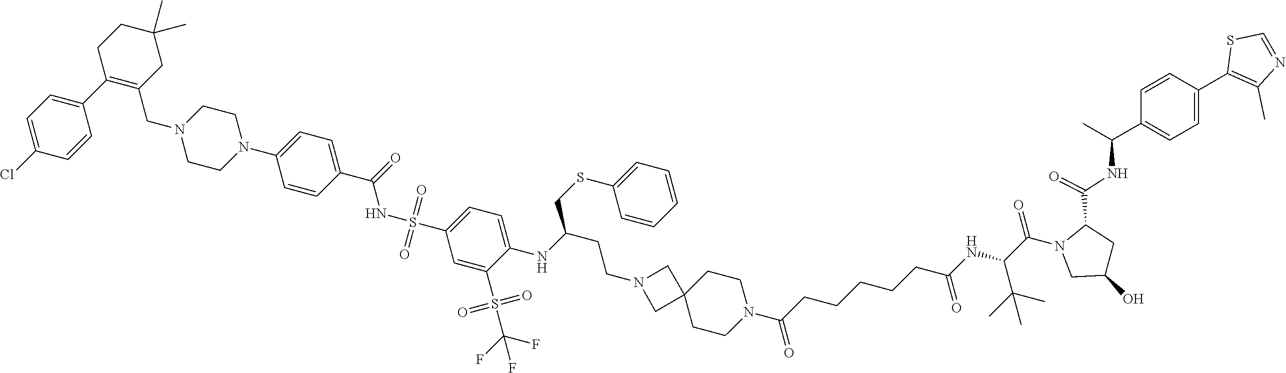 [SMILES: Cc1ncsc1-c1ccc([C@H](C)NC(=O)[C@@H]2C[C@@H](O)CN2C(=O)[C@@H](NC(=O)CCCCCC(=O)N2CCC3(CC2)CN(CC[C@H](CSc2ccccc2)Nc2ccc(S(=O)(=O)NC(=O)c4ccc(N5CCN(CC6=C(c7ccc(Cl)cc7)CCC(C)(C)C6)CC5)cc4)cc2S(=O)(=O)C(F)(F)F)C3)C(C)(C)C)cc1]

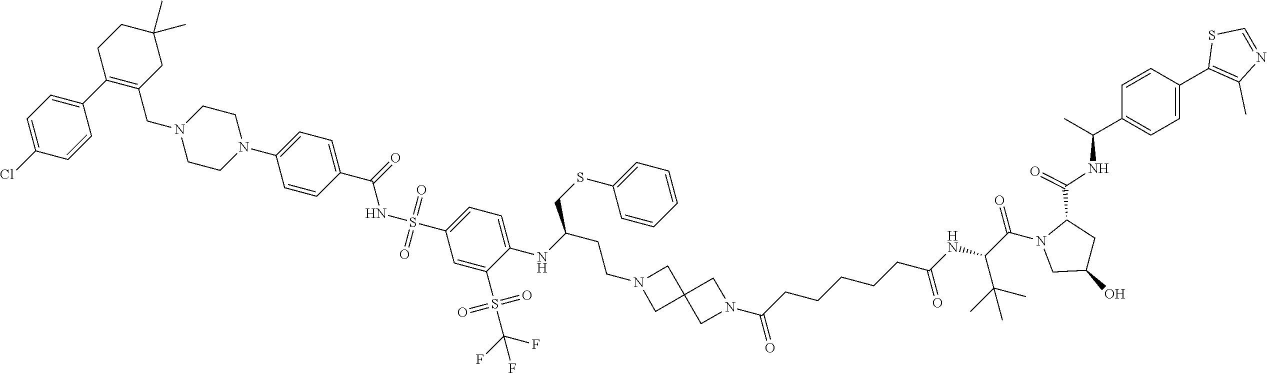 [SMILES: Cc1ncsc1-c1ccc([C@H](C)NC(=O)[C@@H]2C[C@@H](O)CN2C(=O)[C@@H](NC(=O)CCCCCC(=O)N2CC3(CN(CC[C@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)C3)C2)C(C)(C)C)cc1]

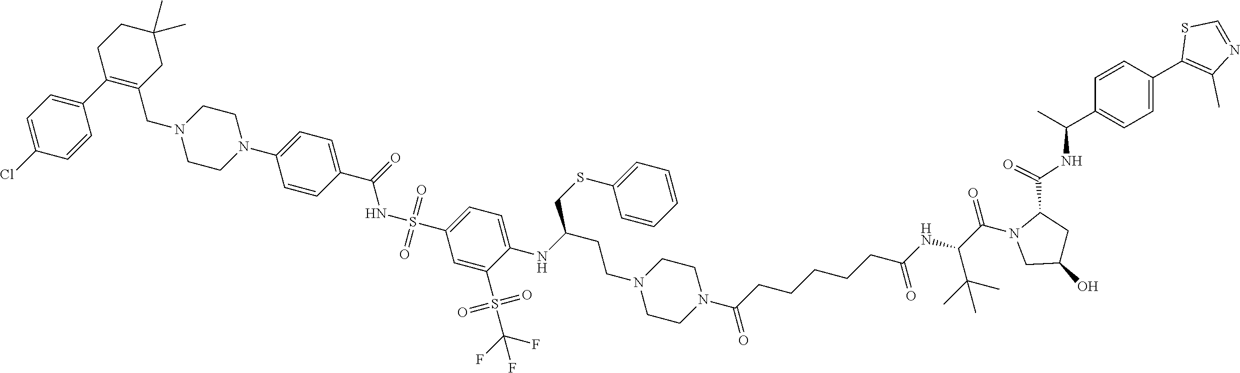 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)(C)C)cc1]

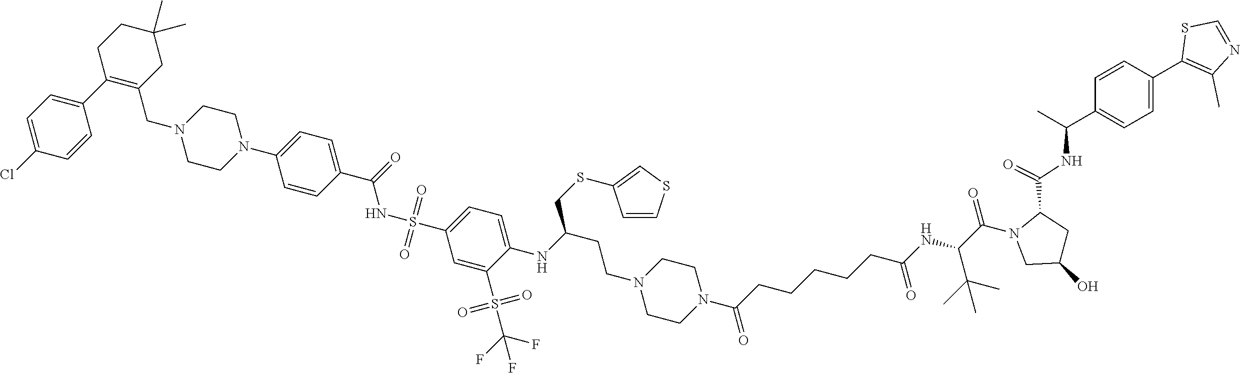 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccsc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)(C)C)cc1]

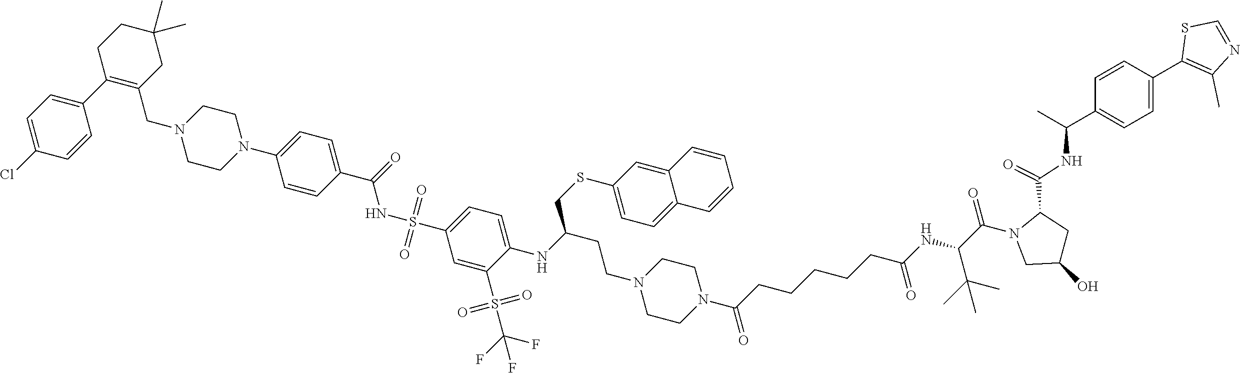 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccc4ccccc4c3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)(C)C)cc1]

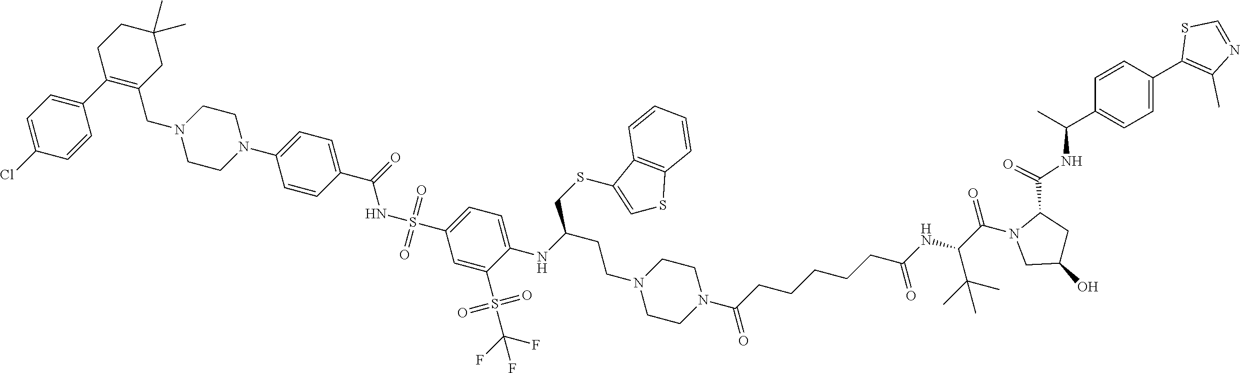 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3csc4ccccc34)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)(C)C)cc1]

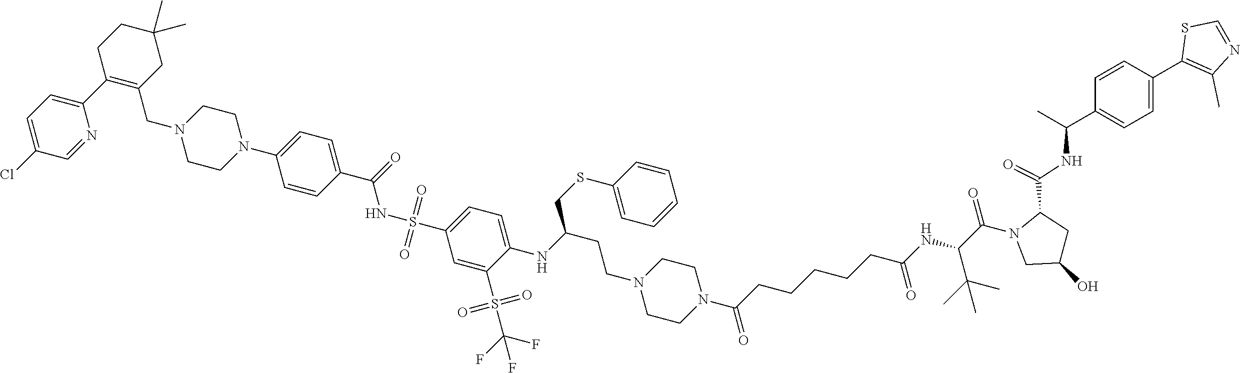 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cn7)CCC(C)(C)C6)CC5)cc4)cc3S(=O)(=O)C(F)(F)F)CC2)C(C)(C)C)cc1]

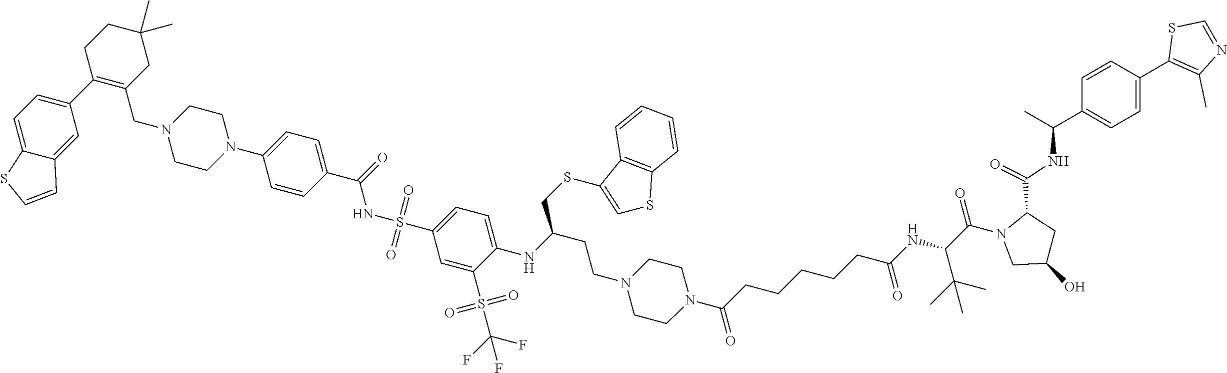 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3csc4ccccc34)Nc3ccc(S(=O)(=O)NC(=O)c4ccc(N5CCN(CC6=C(c7ccc8sccc8c7)CCC(C)(C)C6)CC5)cc4)cc3S(=O)(=O)C(F)(F)F)CC2)C(C)(C)C)cc1]

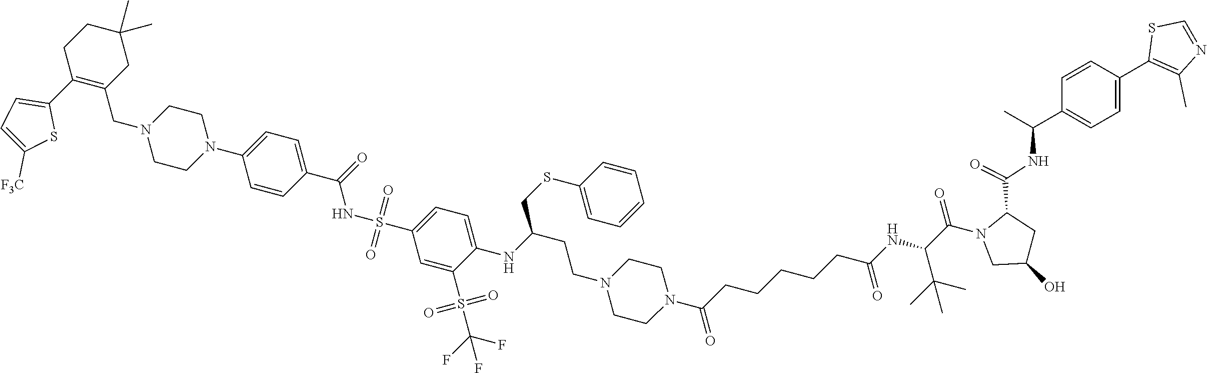 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(C(F)(F)F)s7)CCC(C)(C)C6)CC5)cc4)cc3S(=O)(=O)C(F)(F)F)CC2)C(C)(C)C)cc1]

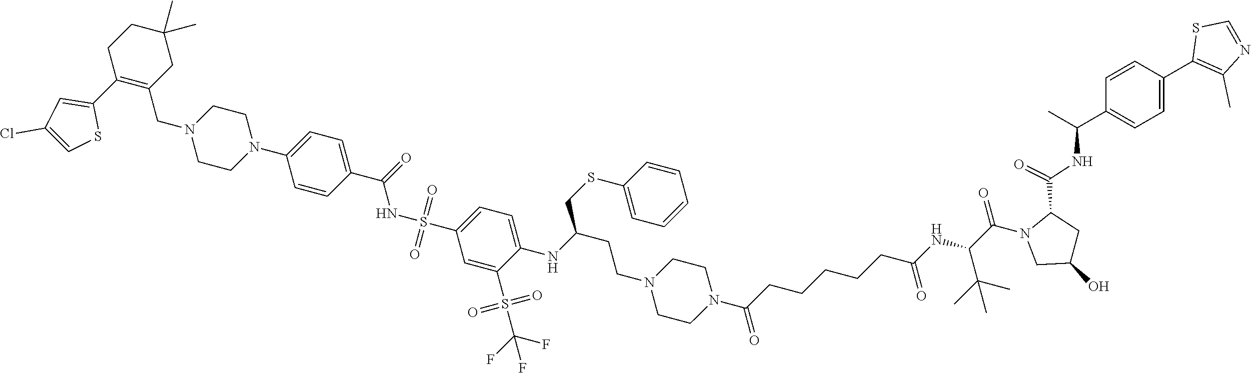 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7cc(Cl)cs7)CCC(C)(C)C6)CC5)cc4)cc3S(=O)(=O)C(F)(F)F)CC2)C(C)(C)C)cc1]

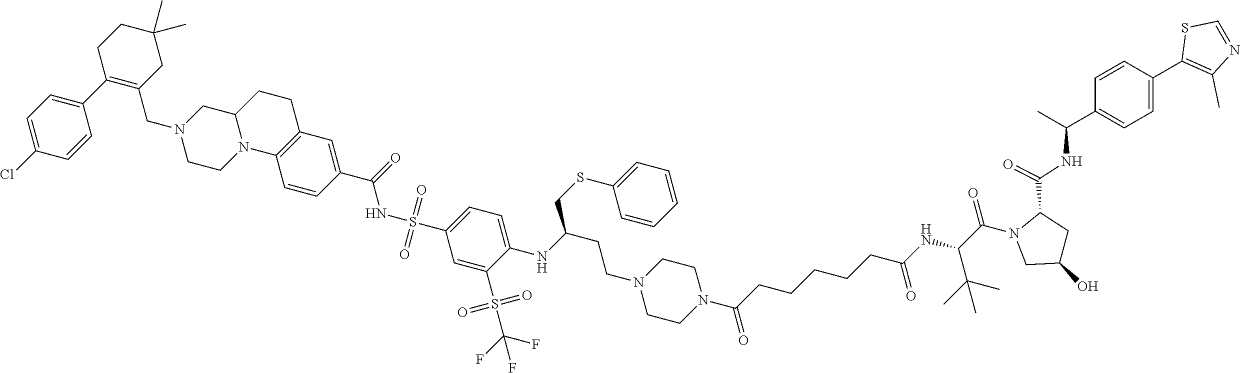 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

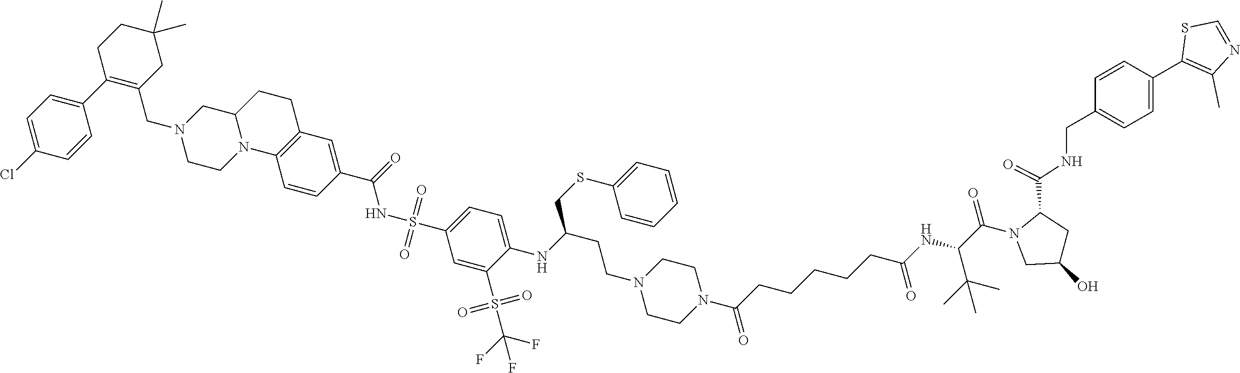 [SMILES: Cc1ncsc1-c1ccc(CNC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

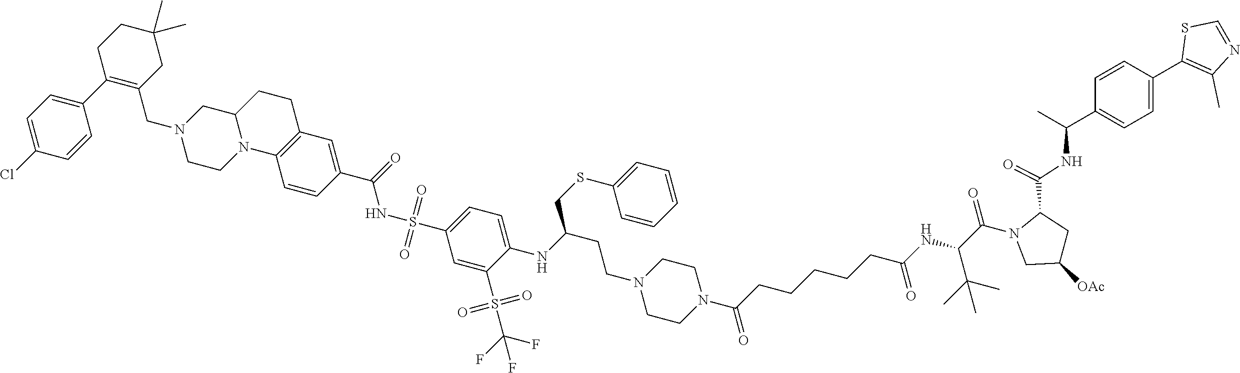 [SMILES: CC(=O)O[C@@H]1C[C@@H](C(=O)N[C@@H](C)c2ccc(-c3scnc3C)cc2)N(C(=O)[C@@H](NC(=O)CCCCCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)C1]

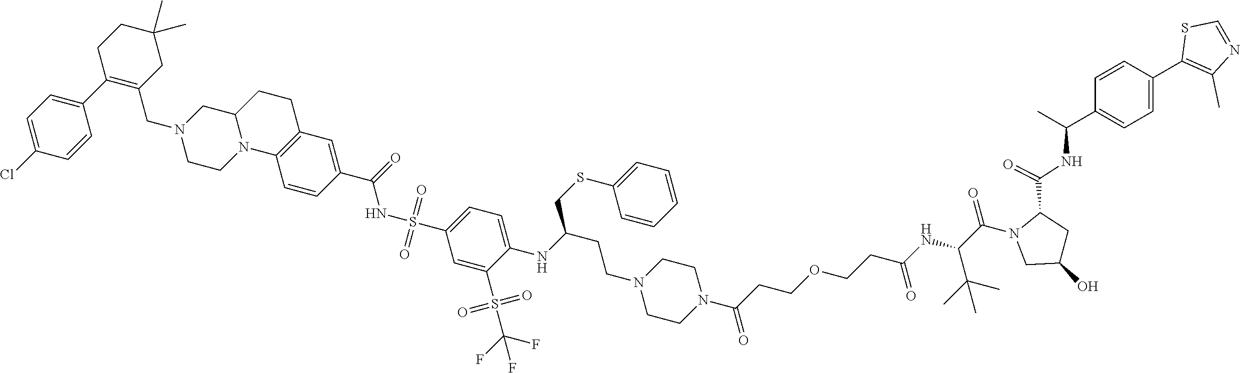 [SMILES: Cc1ncsc1-c1ccc([C@H](C)NC(=O)[C@@H]2C[C@@H](O)CN2C(=O)[C@@H](NC(=O)CCOCCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

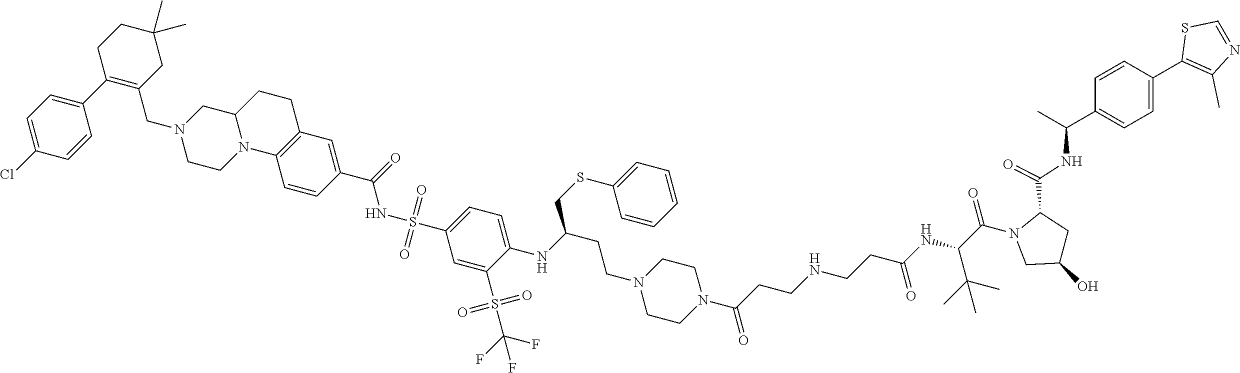 [SMILES: Cc1ncsc1-c1ccc([C@H](C)NC(=O)[C@@H]2C[C@@H](O)CN2C(=O)[C@@H](NC(=O)CCNCCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

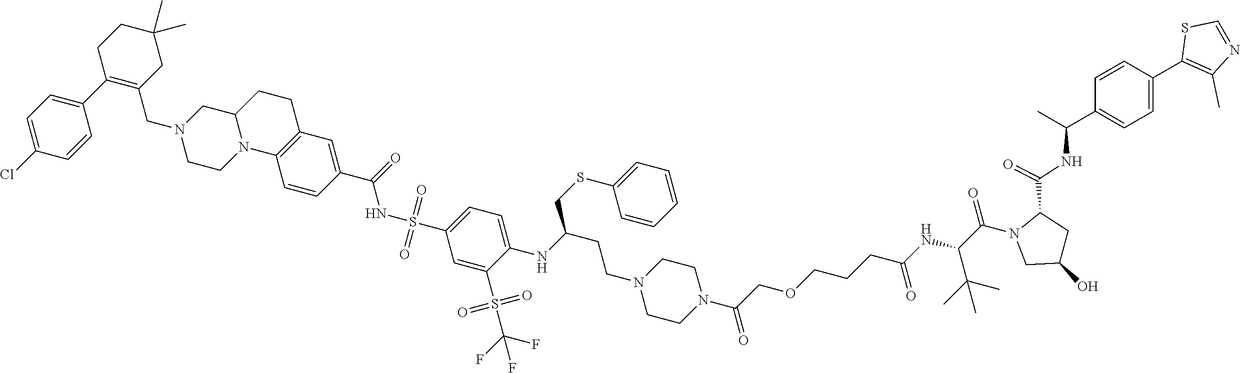 [SMILES: Cc1ncsc1-c1ccc([C@H](C)NC(=O)[C@@H]2C[C@@H](O)CN2C(=O)[C@@H](NC(=O)CCCOCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

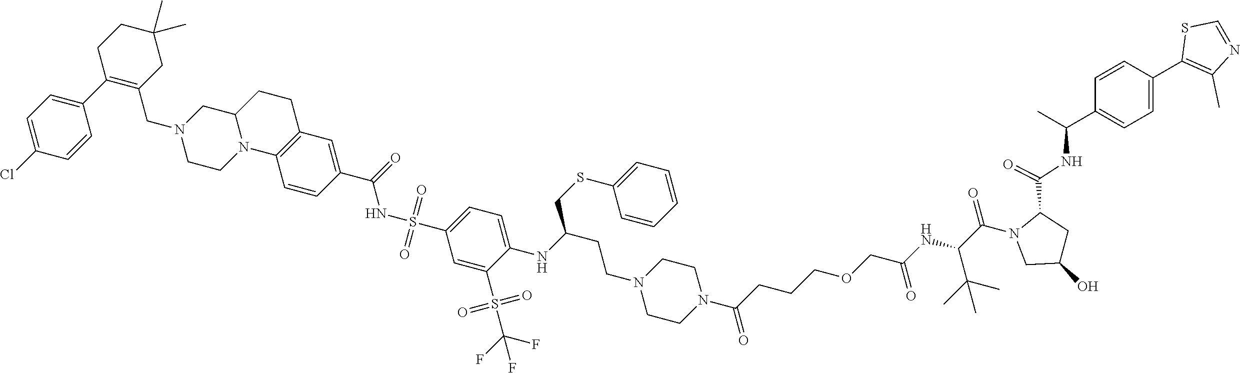 [SMILES: Cc1ncsc1-c1ccc([C@H](C)NC(=O)[C@@H]2C[C@@H](O)CN2C(=O)[C@@H](NC(=O)COCCCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

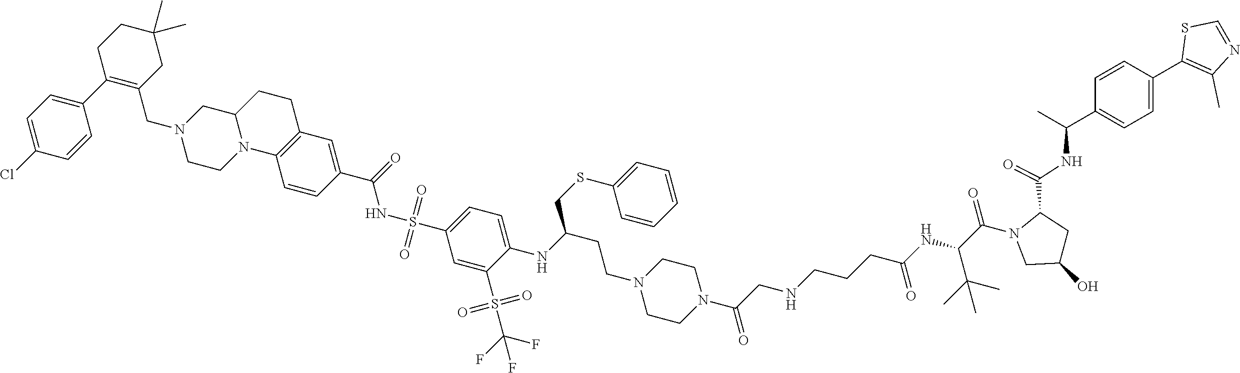 [SMILES: Cc1ncsc1-c1ccc([C@H](C)NC(=O)[C@@H]2C[C@@H](O)CN2C(=O)[C@@H](NC(=O)CCCNCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

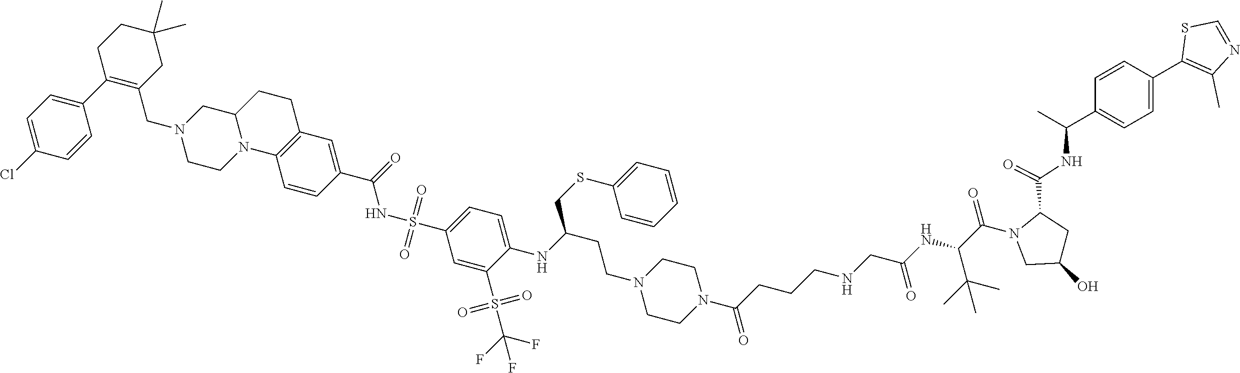 [SMILES: Cc1ncsc1-c1ccc([C@H](C)NC(=O)[C@@H]2C[C@@H](O)CN2C(=O)[C@@H](NC(=O)CNCCCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

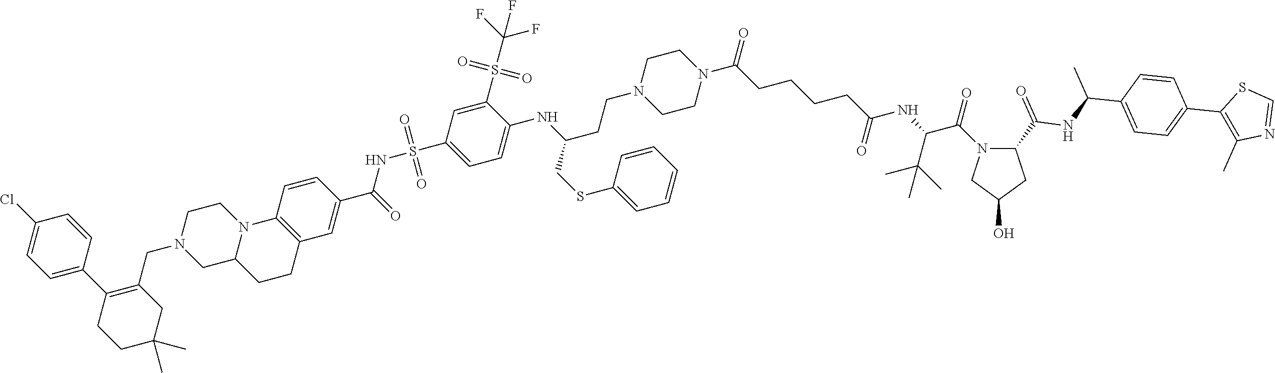 [SMILES: Cc1ncsc1-c1ccc([C@H](C)NC(=O)[C@@H]2C[C@@H](O)CN2C(=O)[C@@H](NC(=O)CCCCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

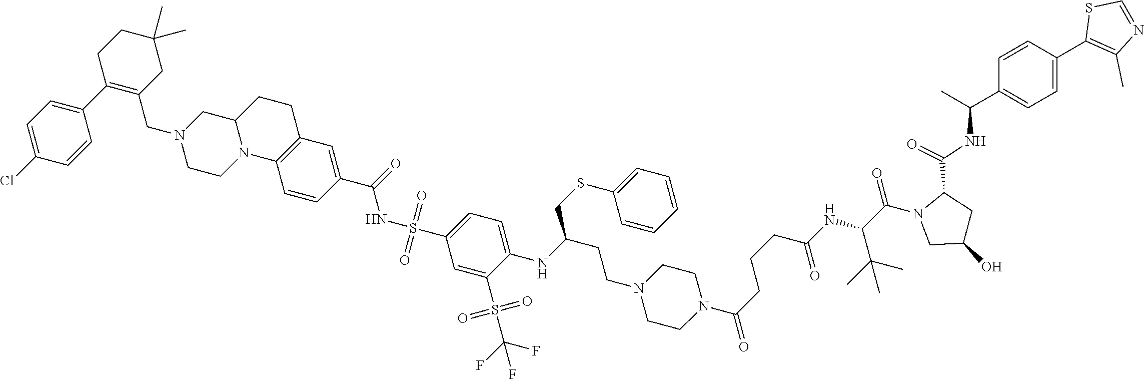 [SMILES: Cc1ncsc1-c1ccc([C@H](C)NC(=O)[C@@H]2C[C@@H](O)CN2C(=O)[C@@H](NC(=O)CCCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

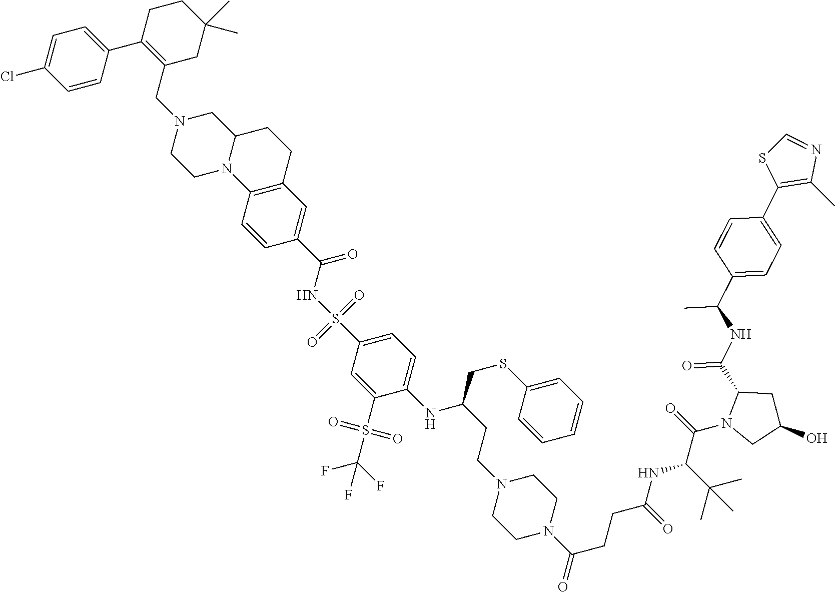 [SMILES: Cc1ncsc1-c1ccc([C@H](C)NC(=O)[C@@H]2C[C@@H](O)CN2C(=O)[C@@H](NC(=O)CCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

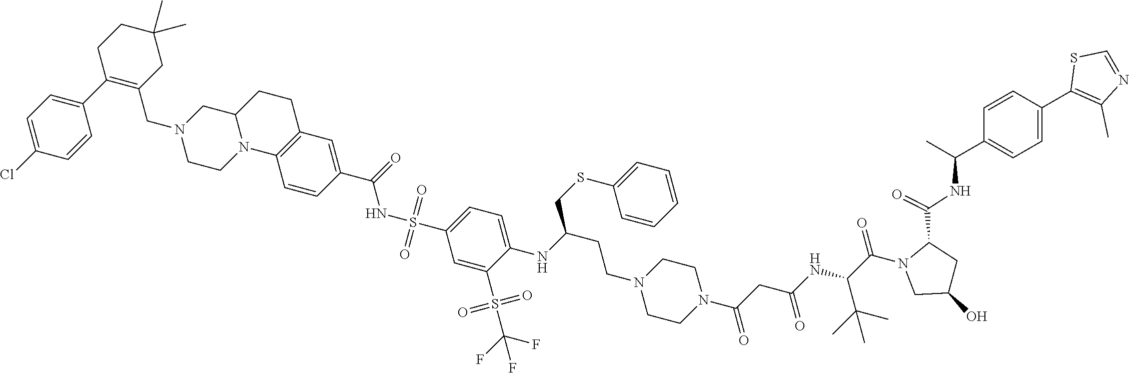 [SMILES: Cc1ncsc1-c1ccc([C@H](C)NC(=O)[C@@H]2C[C@@H](O)CN2C(=O)[C@@H](NC(=O)CC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

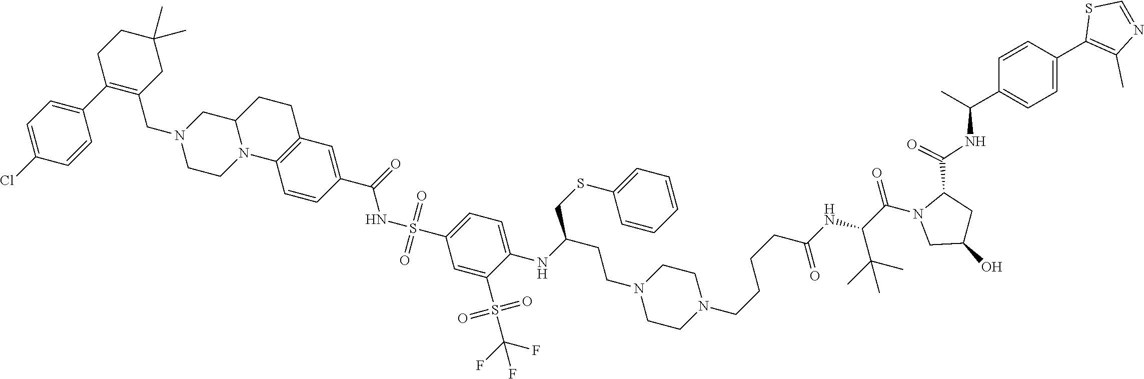 [SMILES: Cc1ncsc1-c1ccc([C@H](C)NC(=O)[C@@H]2C[C@@H](O)CN2C(=O)[C@@H](NC(=O)CCCCN2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

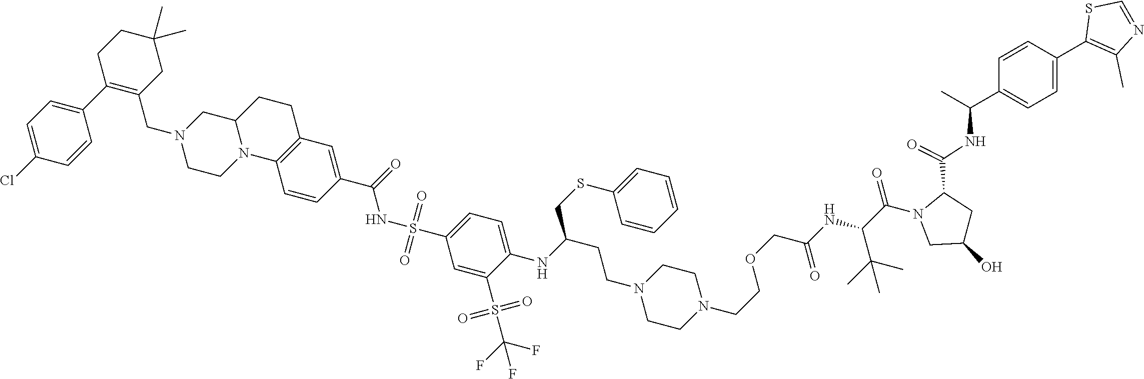 [SMILES: Cc1ncsc1-c1ccc([C@H](C)NC(=O)[C@@H]2C[C@@H](O)CN2C(=O)[C@@H](NC(=O)COCCN2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

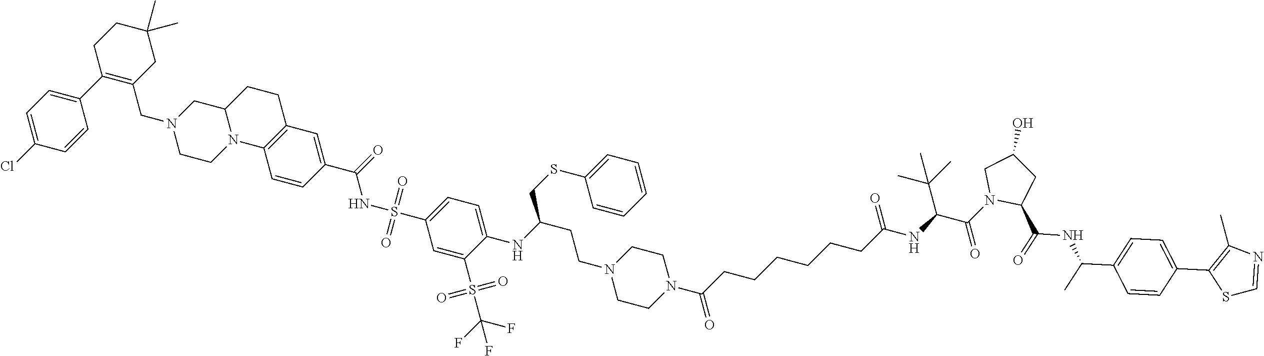 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

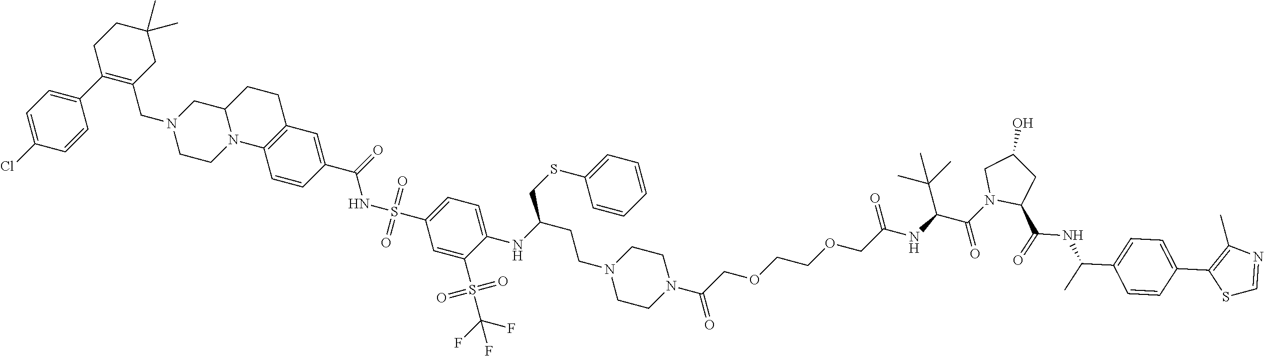 [SMILES: Cc1ncsc1-c1ccc([C@H](C)NC(=O)[C@@H]2C[C@@H](O)CN2C(=O)[C@@H](NC(=O)COCCOCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

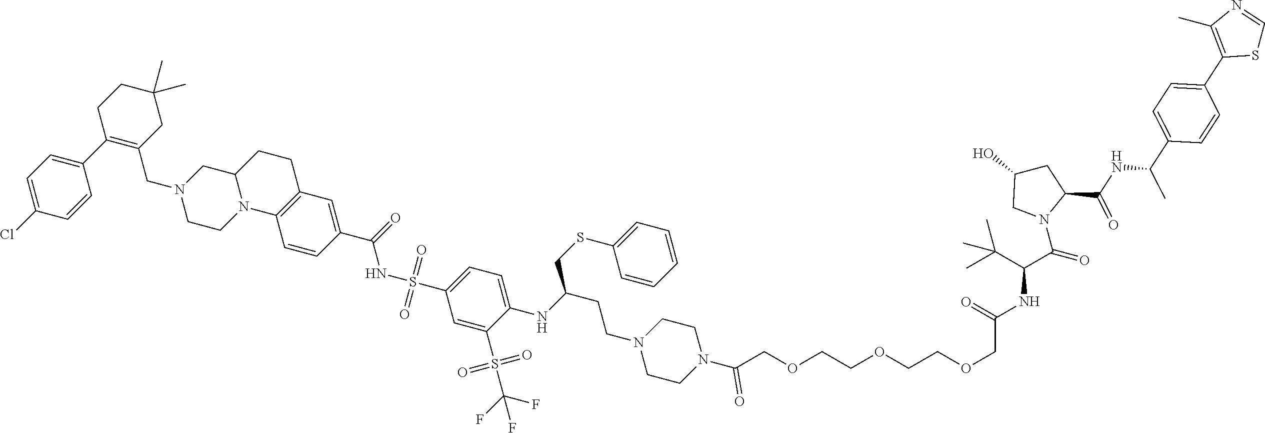 [SMILES: Cc1ncsc1-c1ccc([C@H](C)NC(=O)[C@@H]2C[C@@H](O)CN2C(=O)[C@@H](NC(=O)COCCOCCOCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

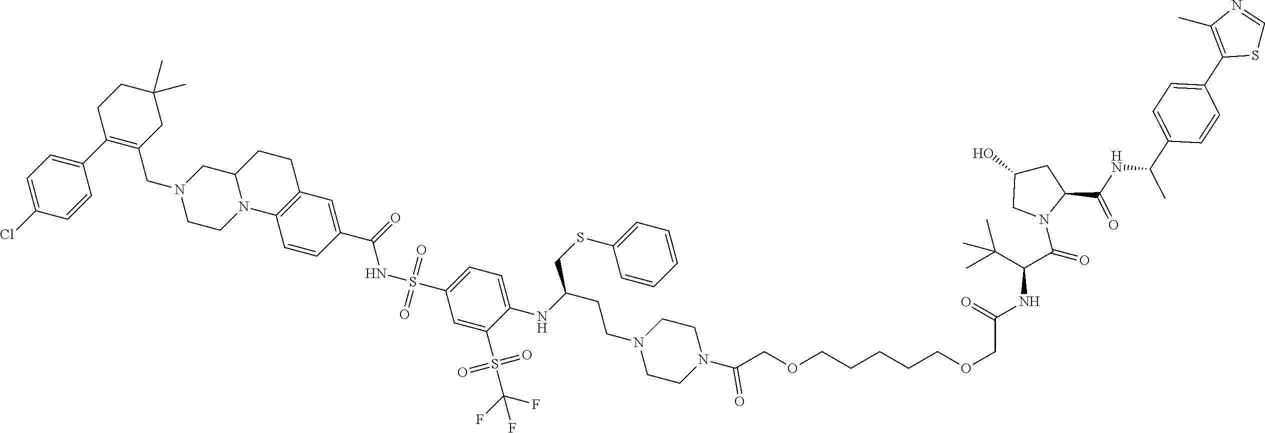 [SMILES: Cc1ncsc1-c1ccc([C@H](C)NC(=O)[C@@H]2C[C@@H](O)CN2C(=O)[C@@H](NC(=O)COCCCCCOCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

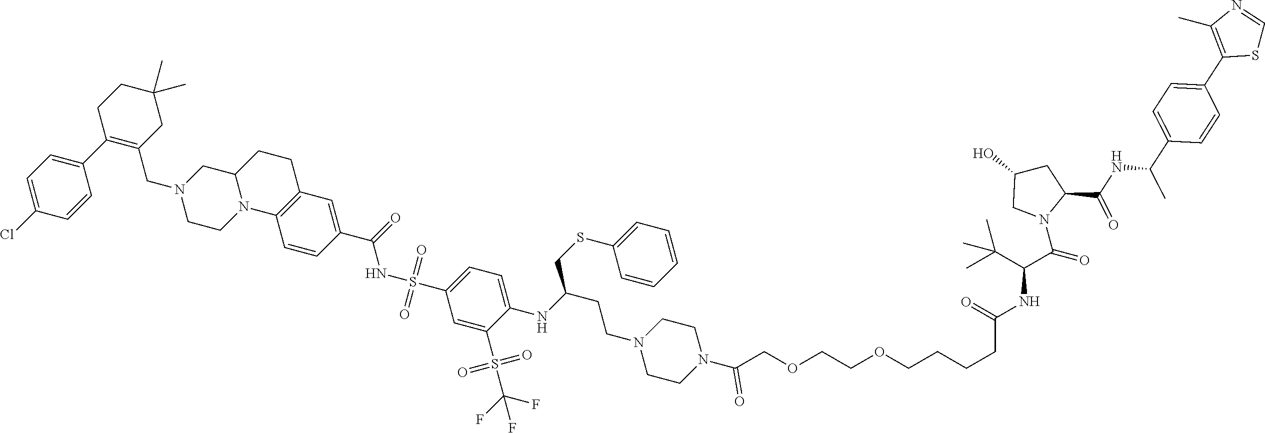 [SMILES: Cc1ncsc1-c1ccc([C@H](C)NC(=O)[C@@H]2C[C@@H](O)CN2C(=O)[C@@H](NC(=O)CCCCOCCOCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

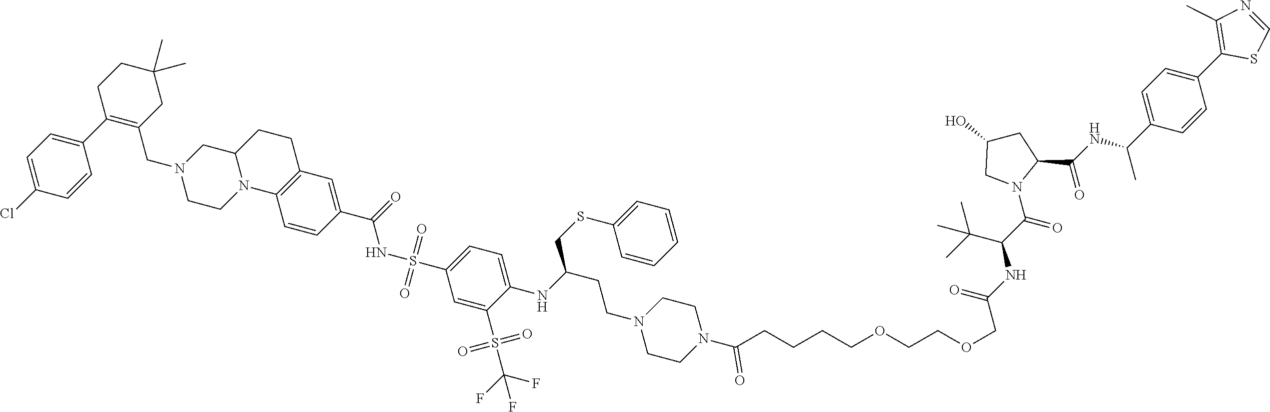 [SMILES: Cc1ncsc1-c1ccc([C@H](C)NC(=O)[C@@H]2C[C@@H](O)CN2C(=O)[C@@H](NC(=O)COCCOCCCCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

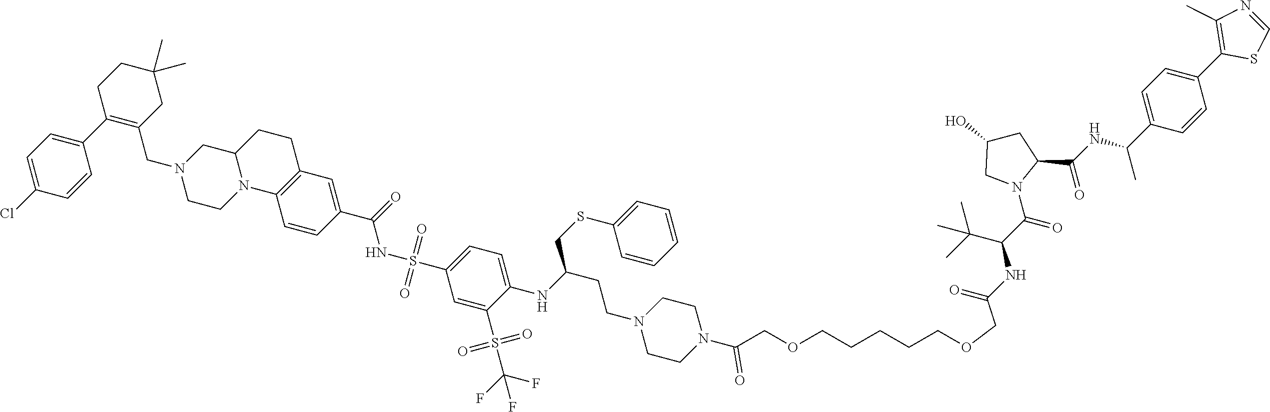 [SMILES: Cc1ncsc1-c1ccc([C@H](C)NC(=O)[C@@H]2C[C@@H](O)CN2C(=O)[C@@H](NC(=O)COCCCCCOCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

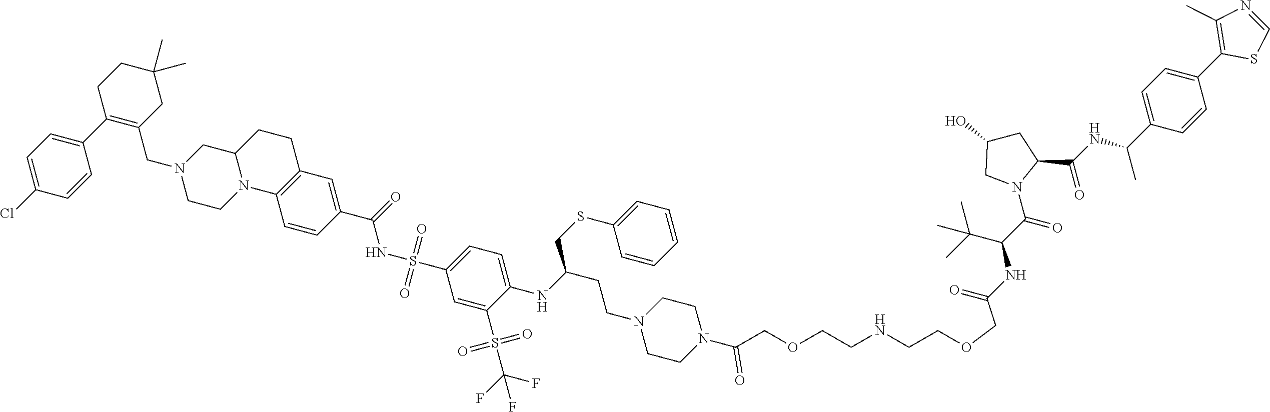 [SMILES: Cc1ncsc1-c1ccc([C@H](C)NC(=O)[C@@H]2C[C@@H](O)CN2C(=O)[C@@H](NC(=O)COCCNCCOCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

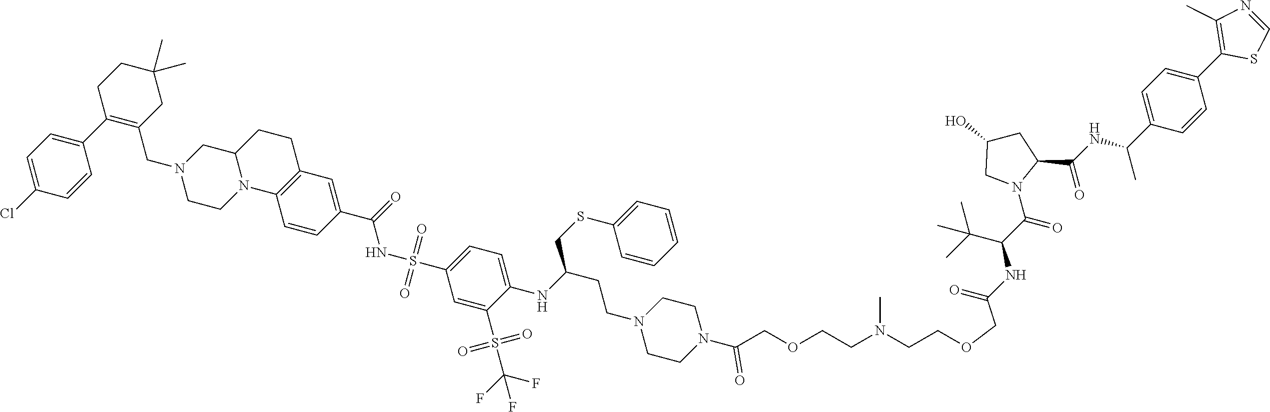 [SMILES: Cc1ncsc1-c1ccc([C@H](C)NC(=O)[C@@H]2C[C@@H](O)CN2C(=O)[C@@H](NC(=O)COCCN(C)CCOCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

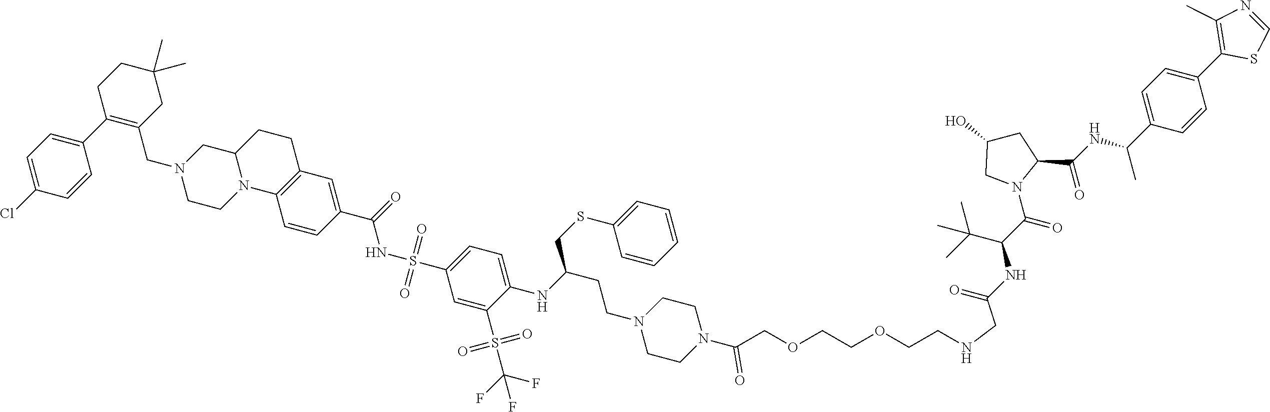 [SMILES: Cc1ncsc1-c1ccc([C@H](C)NC(=O)[C@@H]2C[C@@H](O)CN2C(=O)[C@@H](NC(=O)CNCCOCCOCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

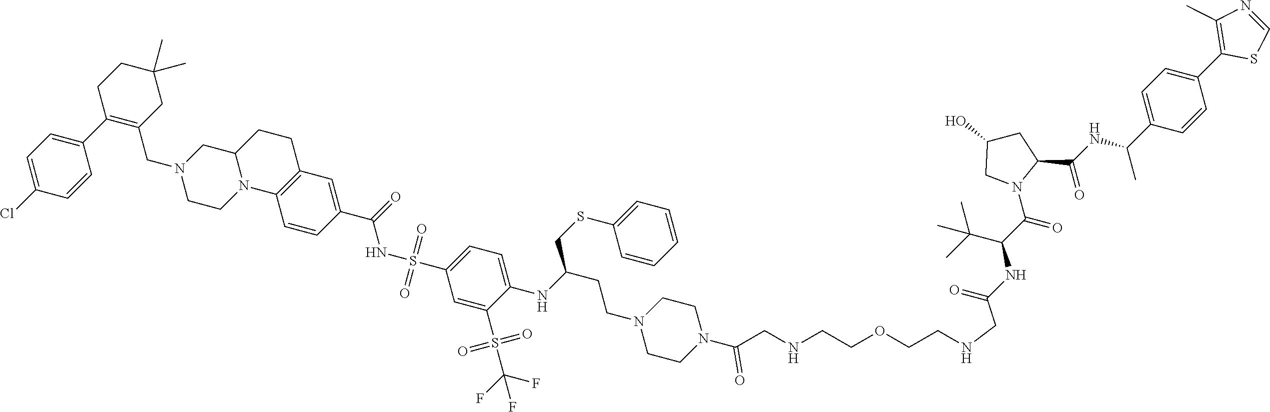 [SMILES: Cc1ncsc1-c1ccc([C@H](C)NC(=O)[C@@H]2C[C@@H](O)CN2C(=O)[C@@H](NC(=O)CNCCOCCNCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

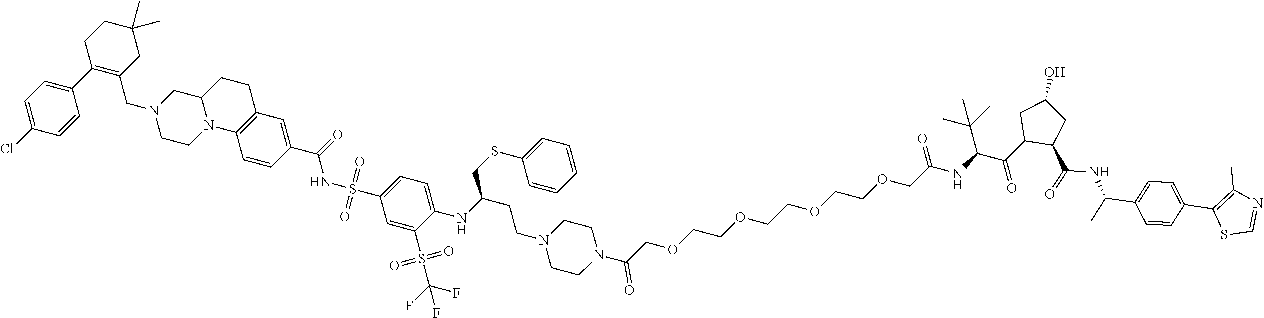 [SMILES: Cc1ncsc1-c1ccc([C@H](C)NC(=O)[C@@H]2C[C@@H](O)CC2C(=O)[C@@H](NC(=O)COCCOCCOCCOCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

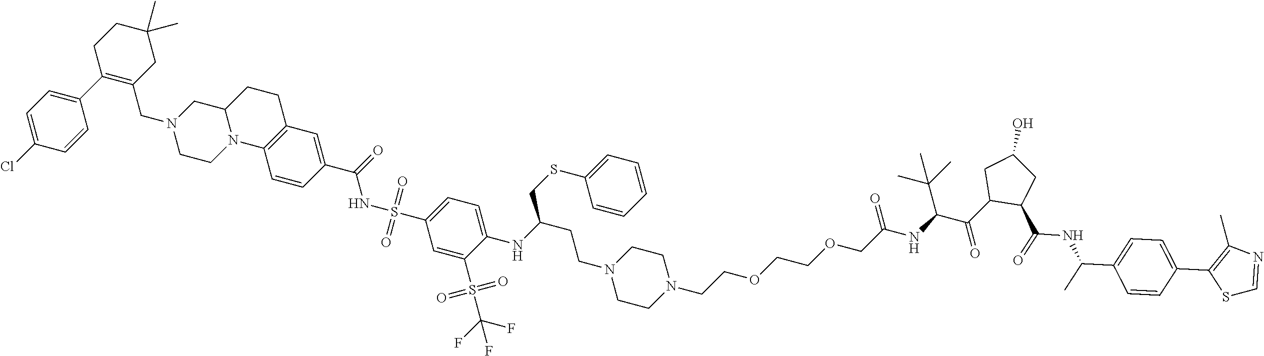 [SMILES: Cc1ncsc1-c1ccc([C@H](C)NC(=O)[C@@H]2C[C@@H](O)CC2C(=O)[C@@H](NC(=O)COCCOCCN2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

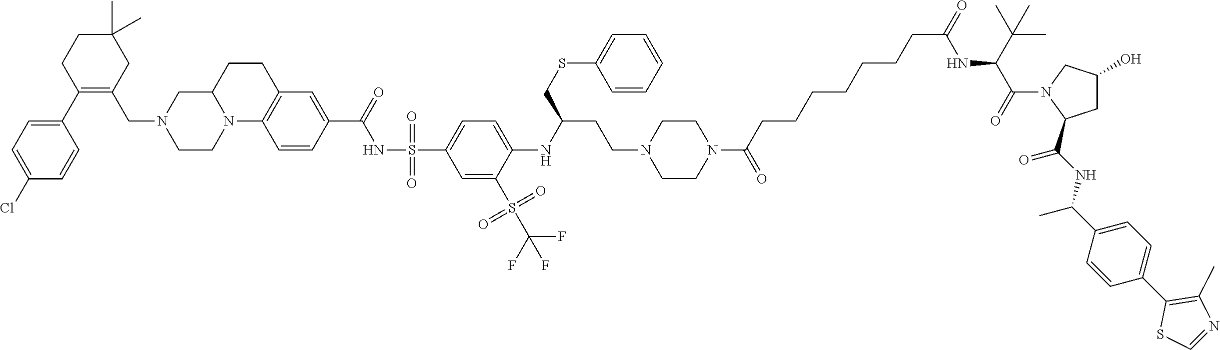 [SMILES: Cc1ncsc1-c1ccc([C@H](C)NC(=O)[C@@H]2C[C@@H](O)CN2C(=O)[C@@H](NC(=O)CCCCCCCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

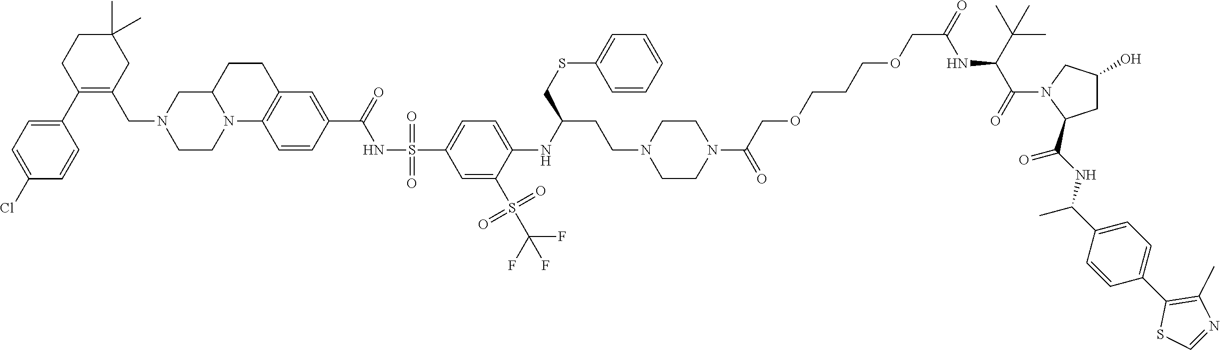 [SMILES: Cc1ncsc1-c1ccc([C@H](C)NC(=O)[C@@H]2C[C@@H](O)CN2C(=O)[C@@H](NC(=O)COCCCOCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

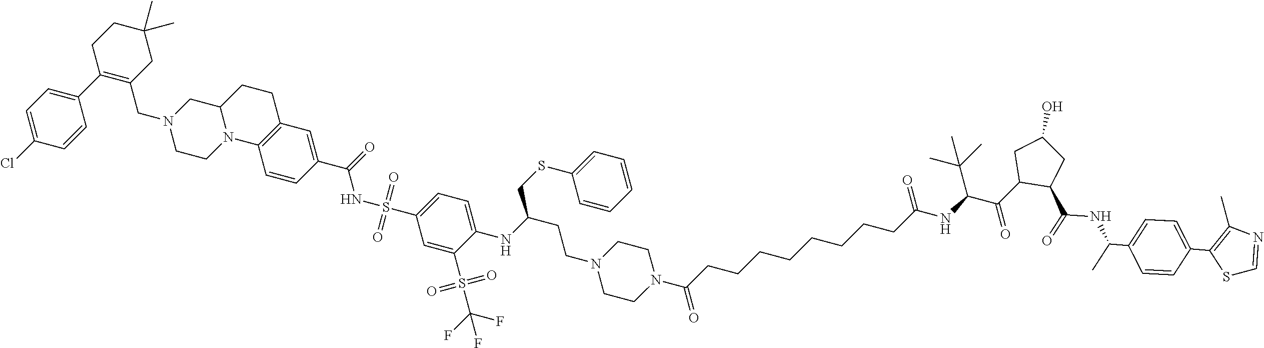 [SMILES: Cc1ncsc1-c1ccc([C@H](C)NC(=O)[C@@H]2C[C@@H](O)CC2C(=O)[C@@H](NC(=O)CCCCCCCCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

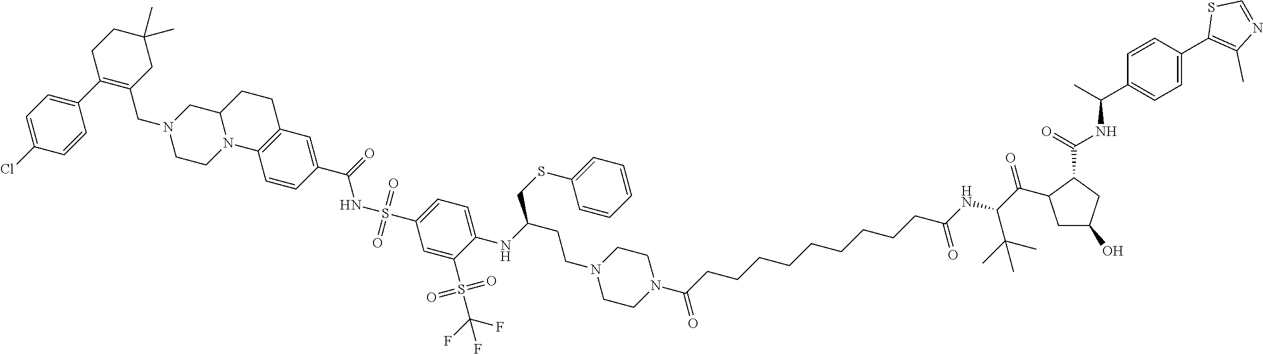 [SMILES: Cc1ncsc1-c1ccc([C@H](C)NC(=O)[C@@H]2C[C@@H](O)CC2C(=O)[C@@H](NC(=O)CCCCCCCCCC(=O)N2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

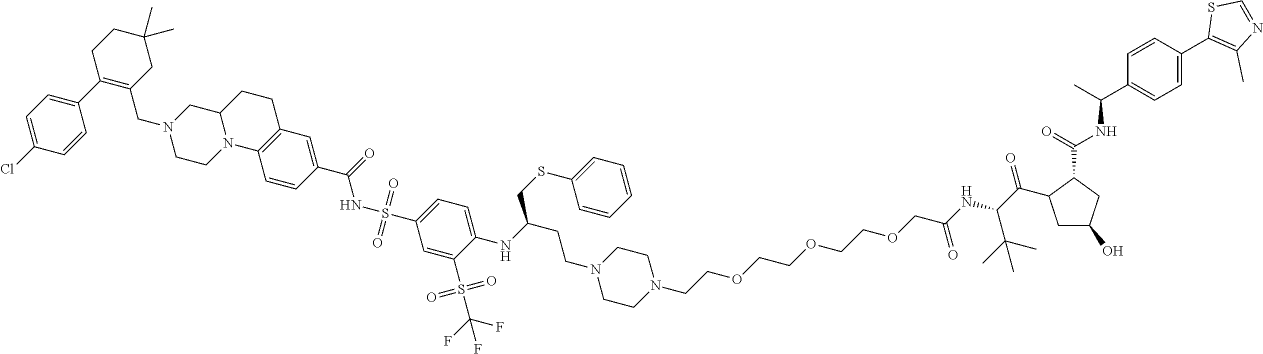 [SMILES: Cc1ncsc1-c1ccc([C@H](C)NC(=O)[C@@H]2C[C@@H](O)CC2C(=O)[C@@H](NC(=O)COCCOCCOCCN2CCN(CC[C@H](CSc3ccccc3)Nc3ccc(S(=O)(=O)NC(=O)c4ccc5c(c4)CCC4CN(CC6=C(c7ccc(Cl)cc7)CCC(C)(C)C6)CCN54)cc3S(=O)(=O)C(F)(F)F)CC2)C(C)(C)C)cc1]

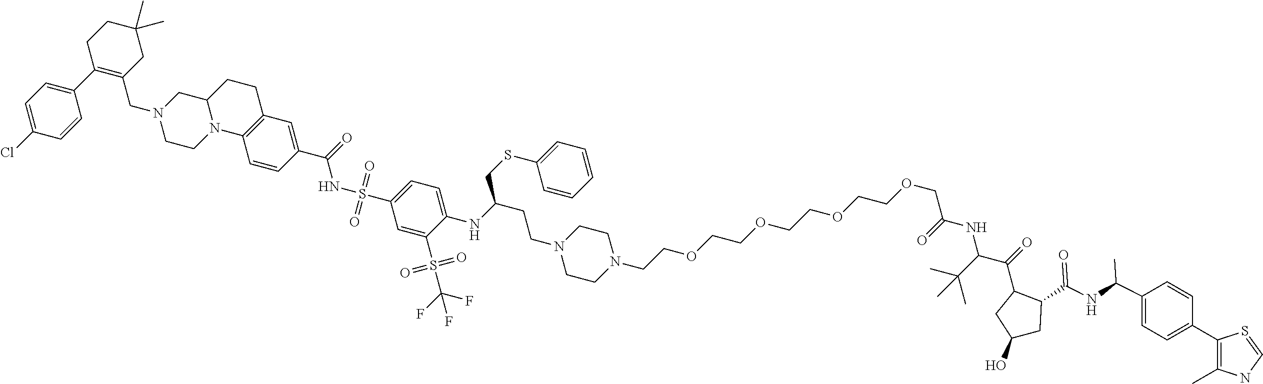

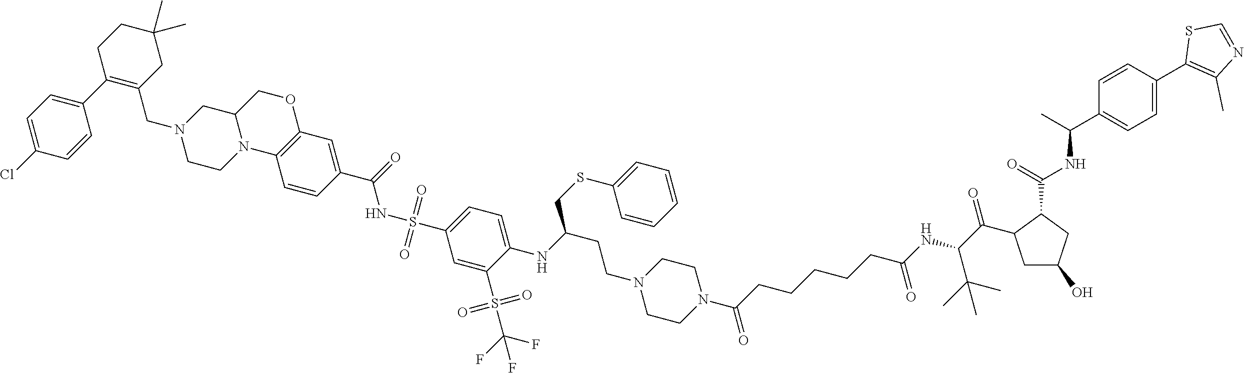 [SMILES: Cc1ncsc1-c1ccc([C@H](C)NC(=O)[C@@H]2C[C@@H](O)CC2C(=O)[C@@H](NC(=O)CCCCCC(=O)N2CCN(CC[C@H](CSc3ccccc3)Nc3ccc(S(=O)(=O)NC(=O)c4ccc5c(c4)OCC4CN(CC6=C(c7ccc(Cl)cc7)CCC(C)(C)C6)CCN54)cc3S(=O)(=O)C(F)(F)F)CC2)C(C)(C)C)cc1]

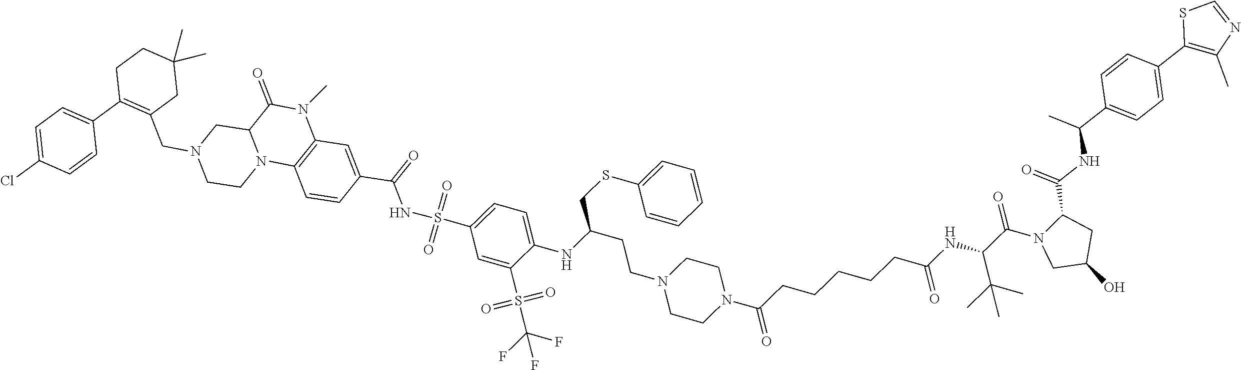 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc5c(c4)N(C)C(=O)C4CN(CC6=C(c7ccc(Cl)cc7)CCC(C)(C)C6)CCN54)cc3S(=O)(=O)C(F)(F)F)CC2)C(C)(C)C)cc1]

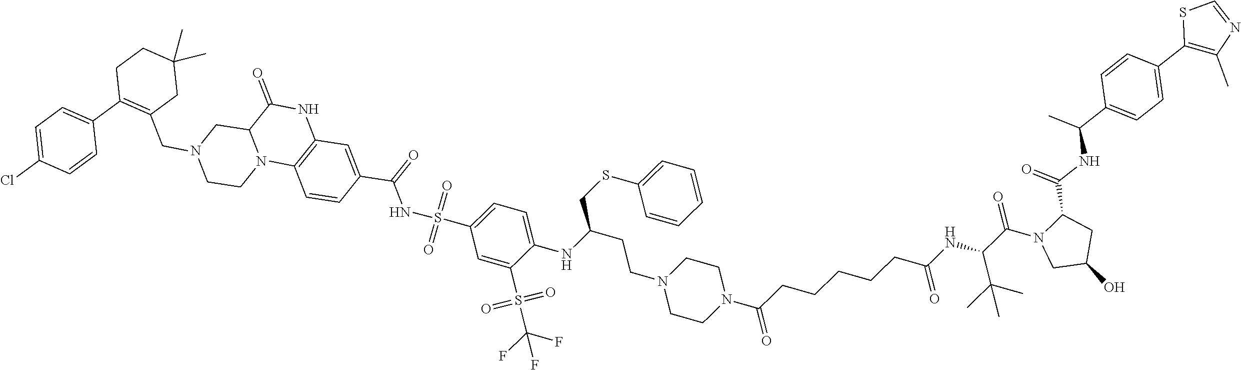 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc5c(c4)NC(=O)C4CN(CC6=C(c7ccc(Cl)cc7)CCC(C)(C)C6)CCN54)cc3S(=O)(=O)C(F)(F)F)CC2)C(C)(C)C)cc1]

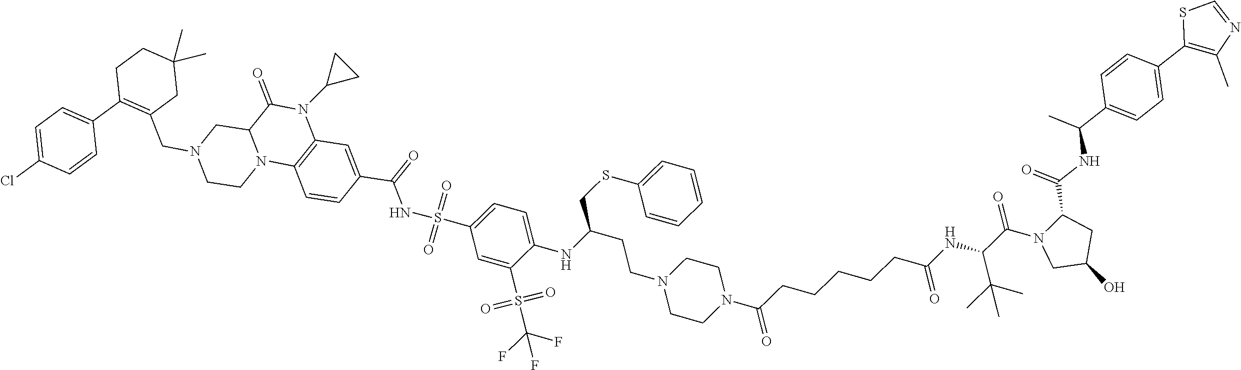 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc5c(c4)N(C4CC4)C(=O)C4CN(CC6=C(c7ccc(Cl)cc7)CCC(C)(C)C6)CCN54)cc3S(=O)(=O)C(F)(F)F)CC2)C(C)(C)C)cc1]

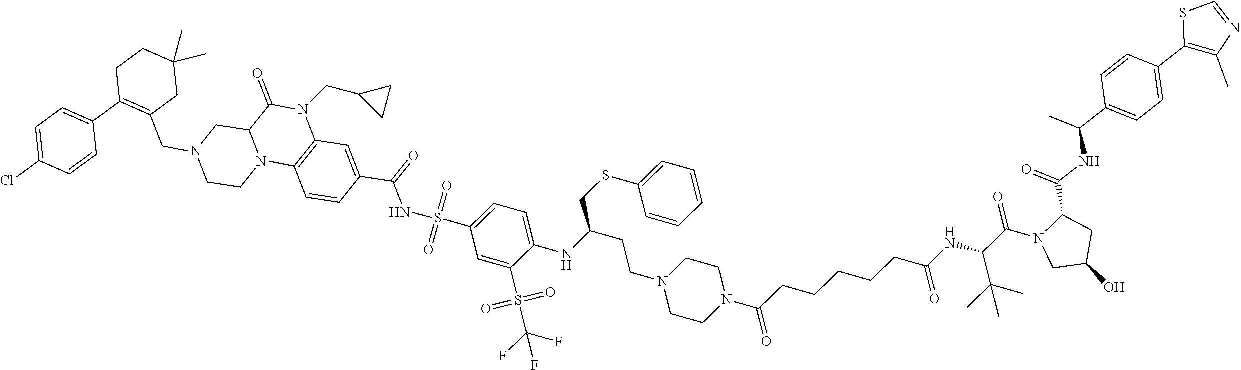 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc5c(c4)N(CC4CC4)C(=O)C4CN(CC6=C(c7ccc(Cl)cc7)CCC(C)(C)C6)CCN54)cc3S(=O)(=O)C(F)(F)F)CC2)C(C)(C)C)cc1]

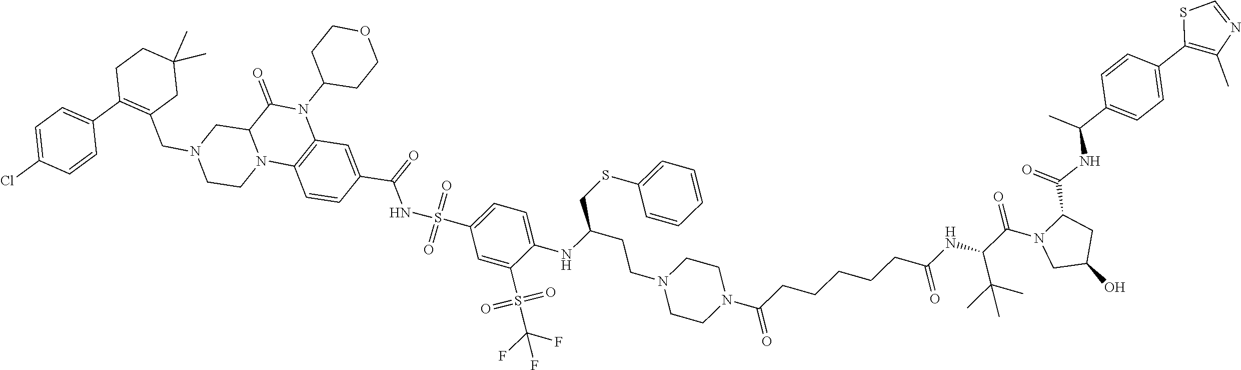 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc5c(c4)N(C4CCOCC4)C(=O)C4CN(CC6=C(c7ccc(Cl)cc7)CCC(C)(C)C6)CCN54)cc3S(=O)(=O)C(F)(F)F)CC2)C(C)(C)C)cc1]

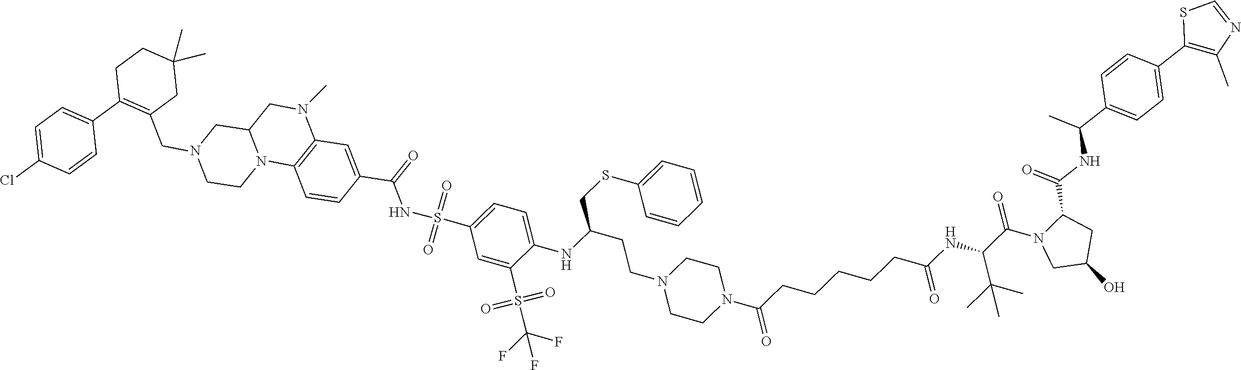 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc5c(c4)N(C)CC4CN(CC6=C(c7ccc(Cl)cc7)CCC(C)(C)C6)CCN54)cc3S(=O)(=O)C(F)(F)F)CC2)C(C)(C)C)cc1]

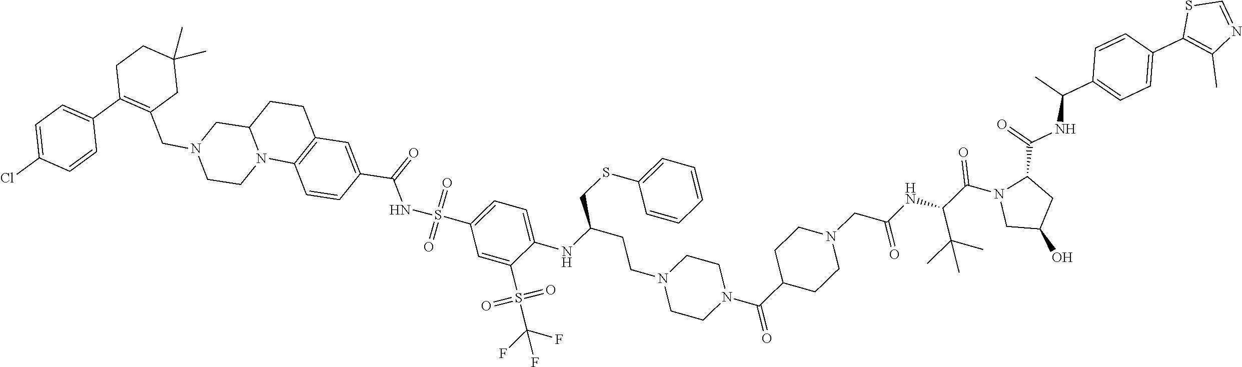 [SMILES: Cc1ncsc1-c1ccc([C@H](C)NC(=O)[C@@H]2C[C@@H](O)CN2C(=O)[C@@H](NC(=O)CN2CCC(C(=O)N3CCN(CC[C@H](CSc4ccccc4)Nc4ccc(S(=O)(=O)NC(=O)c5ccc6c(c5)CCC5CN(CC7=C(c8ccc(Cl)cc8)CCC(C)(C)C7)CCN65)cc4S(=O)(=O)C(F)(F)F)CC3)CC2)C(C)(C)C)cc1]

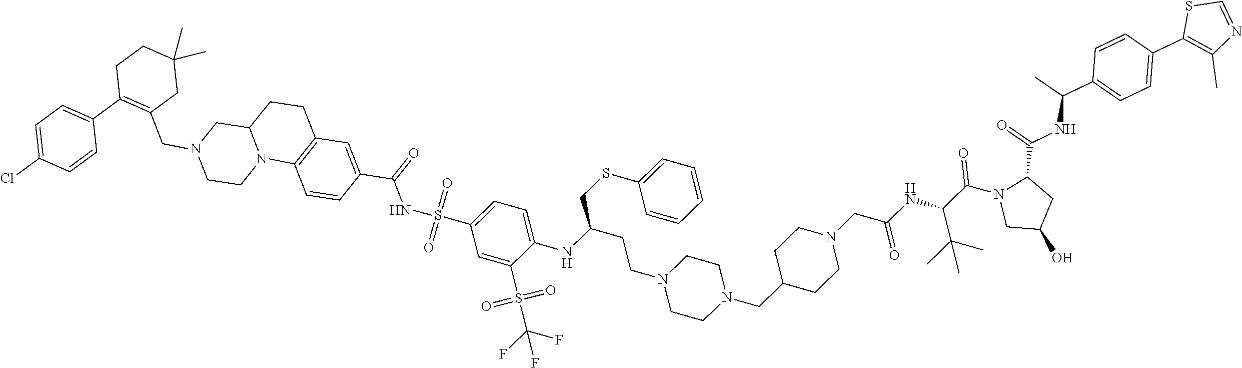 [SMILES: Cc1ncsc1-c1ccc([C@H](C)NC(=O)[C@@H]2C[C@@H](O)CN2C(=O)[C@@H](NC(=O)CN2CCC(CN3CCN(CC[C@H](CSc4ccccc4)Nc4ccc(S(=O)(=O)NC(=O)c5ccc6c(c5)CCC5CN(CC7=C(c8ccc(Cl)cc8)CCC(C)(C)C7)CCN65)cc4S(=O)(=O)C(F)(F)F)CC3)CC2)C(C)(C)C)cc1]

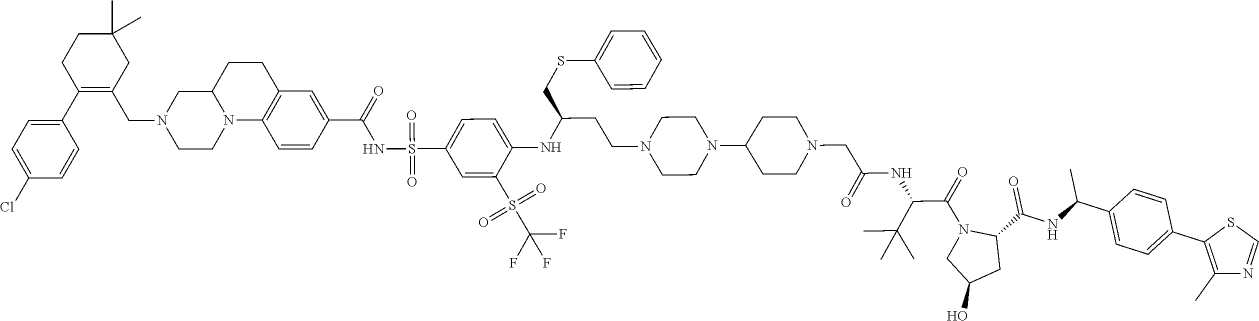 [SMILES: Cc1ncsc1-c1ccc([C@H](C)NC(=O)[C@@H]2C[C@@H](O)CN2C(=O)[C@@H](NC(=O)CN2CCC(N3CCN(CC[C@H](CSc4ccccc4)Nc4ccc(S(=O)(=O)NC(=O)c5ccc6c(c5)CCC5CN(CC7=C(c8ccc(Cl)cc8)CCC(C)(C)C7)CCN65)cc4S(=O)(=O)C(F)(F)F)CC3)CC2)C(C)(C)C)cc1]

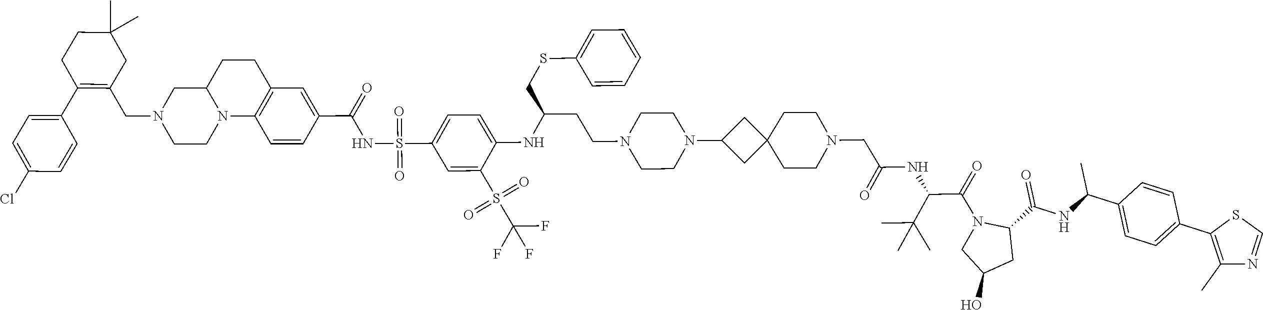 [SMILES: Cc1ncsc1-c1ccc([C@H](C)NC(=O)[C@@H]2C[C@@H](O)CN2C(=O)[C@@H](NC(=O)CN2CCC3(CC2)CC(N2CCN(CC[C@H](CSc4ccccc4)Nc4ccc(S(=O)(=O)NC(=O)c5ccc6c(c5)CCC5CN(CC7=C(c8ccc(Cl)cc8)CCC(C)(C)C7)CCN65)cc4S(=O)(=O)C(F)(F)F)CC2)C3)C(C)(C)C)cc1]

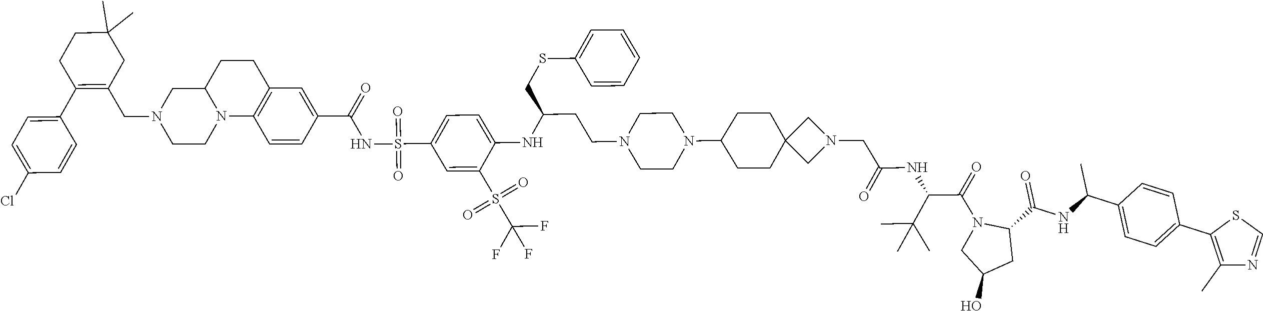 [SMILES: Cc1ncsc1-c1ccc([C@H](C)NC(=O)[C@@H]2C[C@@H](O)CN2C(=O)[C@@H](NC(=O)CN2CC3(CCC(N4CCN(CC[C@H](CSc5ccccc5)Nc5ccc(S(=O)(=O)NC(=O)c6ccc7c(c6)CCC6CN(CC8=C(c9ccc(Cl)cc9)CCC(C)(C)C8)CCN76)cc5S(=O)(=O)C(F)(F)F)CC4)CC3)C2)C(C)(C)C)cc1]

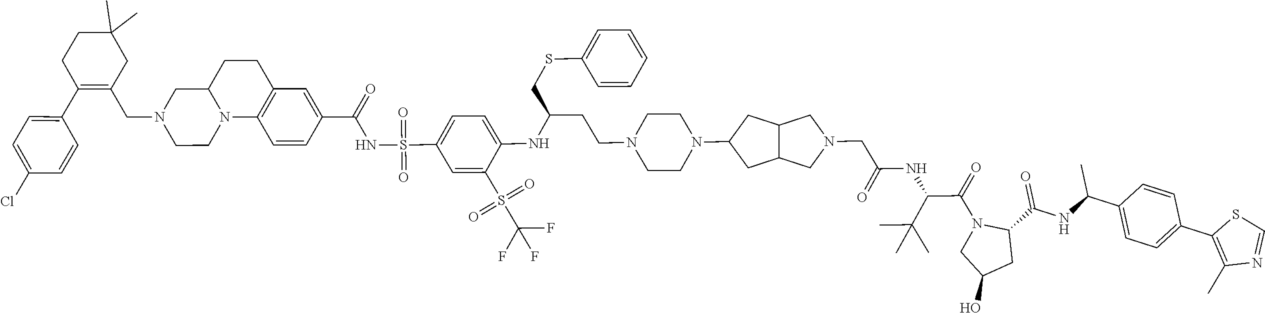 [SMILES: Cc1ncsc1-c1ccc([C@H](C)NC(=O)[C@@H]2C[C@@H](O)CN2C(=O)[C@@H](NC(=O)CN2CC3CC(N4CCN(CC[C@H](CSc5ccccc5)Nc5ccc(S(=O)(=O)NC(=O)c6ccc7c(c6)CCC6CN(CC8=C(c9ccc(Cl)cc9)CCC(C)(C)C8)CCN76)cc5S(=O)(=O)C(F)(F)F)CC4)CC3C2)C(C)(C)C)cc1]

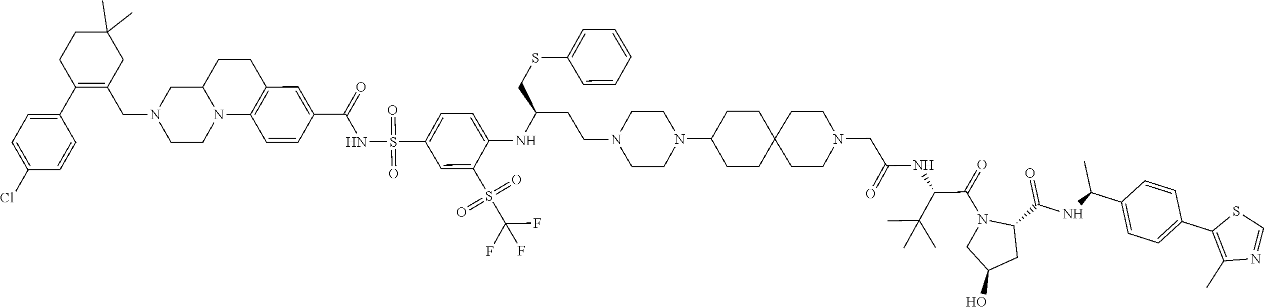 [SMILES: Cc1ncsc1-c1ccc([C@H](C)NC(=O)[C@@H]2C[C@@H](O)CN2C(=O)[C@@H](NC(=O)CN2CCC3(CCC(N4CCN(CC[C@H](CSc5ccccc5)Nc5ccc(S(=O)(=O)NC(=O)c6ccc7c(c6)CCC6CN(CC8=C(c9ccc(Cl)cc9)CCC(C)(C)C8)CCN76)cc5S(=O)(=O)C(F)(F)F)CC4)CC3)CC2)C(C)(C)C)cc1]

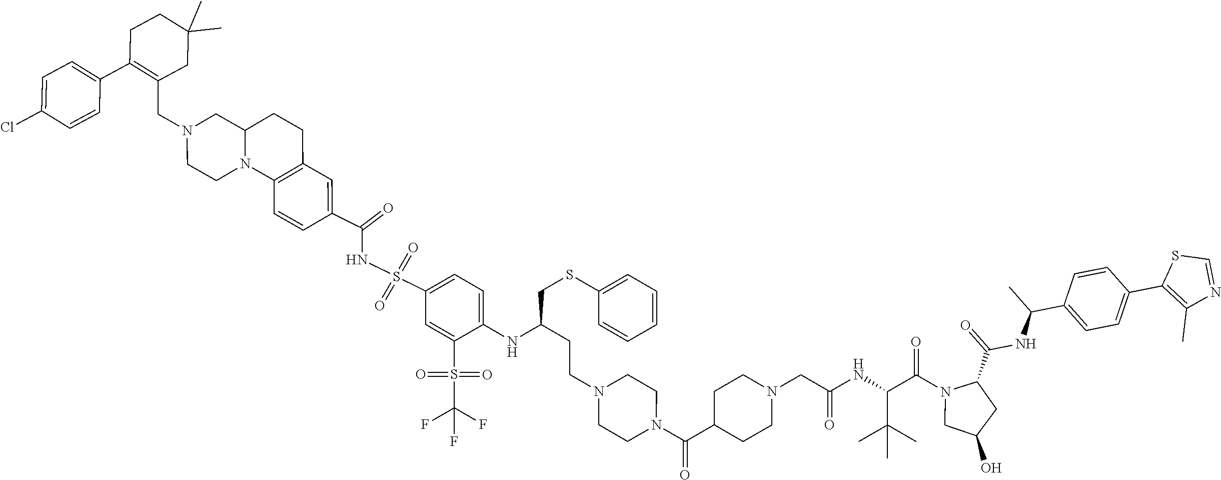 [SMILES: Cc1ncsc1-c1ccc([C@H](C)NC(=O)[C@@H]2C[C@@H](O)CN2C(=O)[C@@H](NC(=O)CN2CCC(C(=O)N3CCN(CC[C@H](CSc4ccccc4)Nc4ccc(S(=O)(=O)NC(=O)c5ccc6c(c5)CCC5CN(CC7=C(c8ccc(Cl)cc8)CCC(C)(C)C7)CCN65)cc4S(=O)(=O)C(F)(F)F)CC3)CC2)C(C)(C)C)cc1]

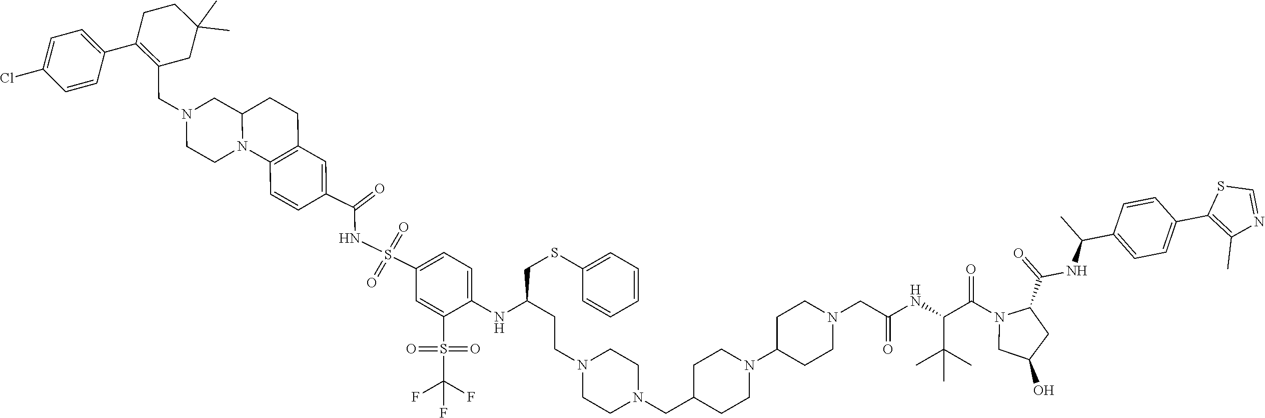 [SMILES: Cc1ncsc1-c1ccc([C@H](C)NC(=O)[C@@H]2C[C@@H](O)CN2C(=O)[C@@H](NC(=O)CN2CCC(N3CCC(CN4CCN(CC[C@H](CSc5ccccc5)Nc5ccc(S(=O)(=O)NC(=O)c6ccc7c(c6)CCC6CN(CC8=C(c9ccc(Cl)cc9)CCC(C)(C)C8)CCN76)cc5S(=O)(=O)C(F)(F)F)CC4)CC3)CC2)C(C)(C)C)cc1]

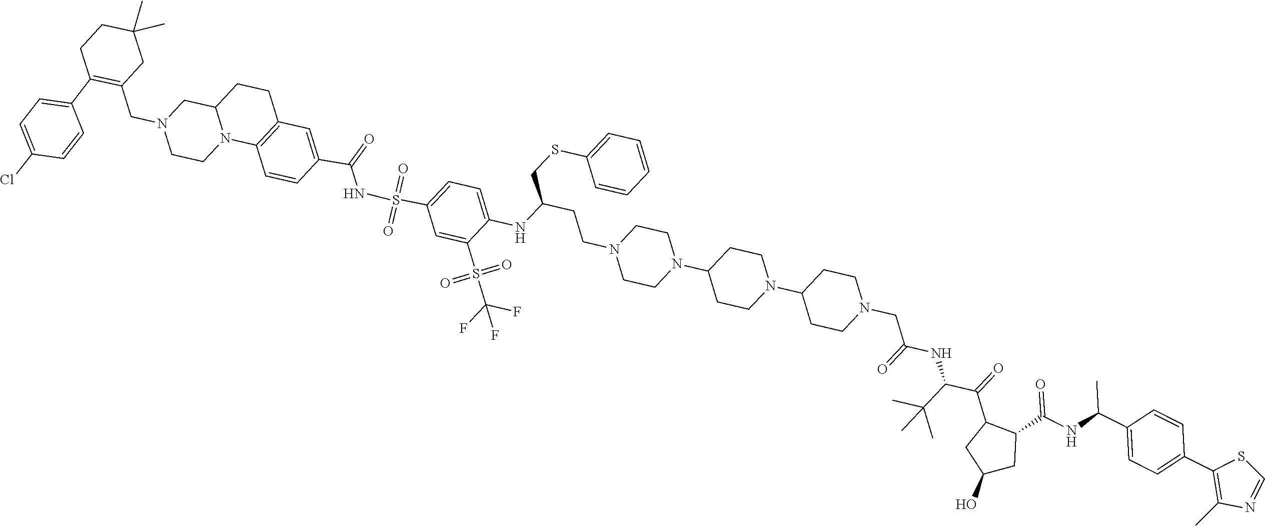 [SMILES: Cc1ncsc1-c1ccc([C@H](C)NC(=O)[C@@H]2C[C@@H](O)CC2C(=O)[C@@H](NC(=O)CN2CCC(N3CCC(N4CCN(CC[C@H](CSc5ccccc5)Nc5ccc(S(=O)(=O)NC(=O)c6ccc7c(c6)CCC6CN(CC8=C(c9ccc(Cl)cc9)CCC(C)(C)C8)CCN76)cc5S(=O)(=O)C(F)(F)F)CC4)CC3)CC2)C(C)(C)C)cc1]

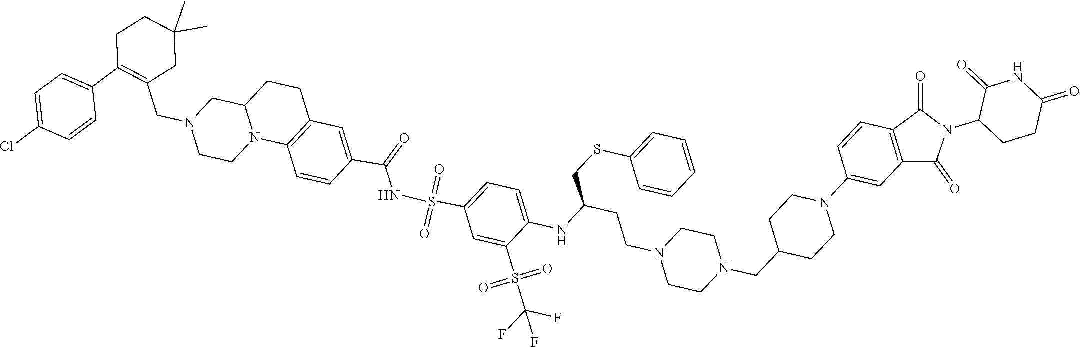 [SMILES: CC1(C)CCC(c2ccc(Cl)cc2)=C(CN2CCN3c4ccc(C(=O)NS(=O)(=O)c5ccc(N[C@H](CCN6CCN(CC7CCN(c8ccc9c(c8)C(=O)N(C8CCC(=O)NC8=O)C9=O)CC7)CC6)CSc6ccccc6)c(S(=O)(=O)C(F)(F)F)c5)cc4CCC3C2)C1]

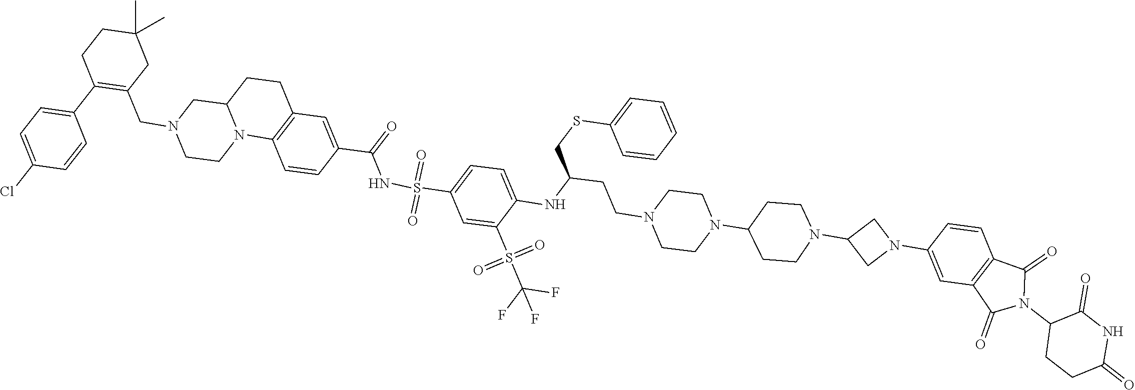 [SMILES: CC1(C)CCC(c2ccc(Cl)cc2)=C(CN2CCN3c4ccc(C(=O)NS(=O)(=O)c5ccc(N[C@H](CCN6CCN(C7CCN(C8CN(c9ccc%10c(c9)C(=O)N(C9CCC(=O)NC9=O)C%10=O)C8)CC7)CC6)CSc6ccccc6)c(S(=O)(=O)C(F)(F)F)c5)cc4CCC3C2)C1]

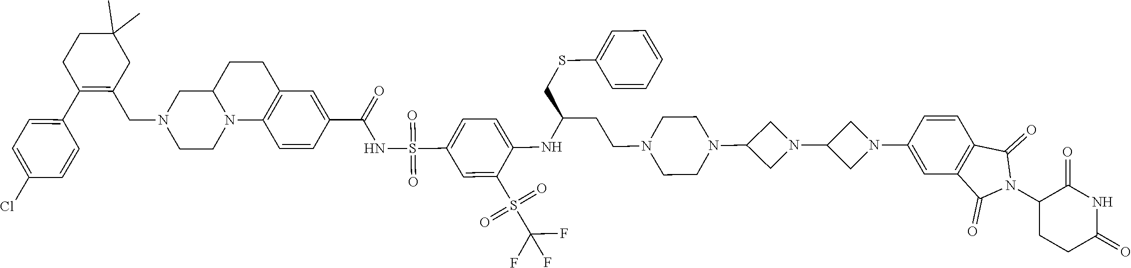 [SMILES: CC1(C)CCC(c2ccc(Cl)cc2)=C(CN2CCN3c4ccc(C(=O)NS(=O)(=O)c5ccc(N[C@H](CCN6CCN(C7CN(C8CN(c9ccc%10c(c9)C(=O)N(C9CCC(=O)NC9=O)C%10=O)C8)C7)CC6)CSc6ccccc6)c(S(=O)(=O)C(F)(F)F)c5)cc4CCC3C2)C1]

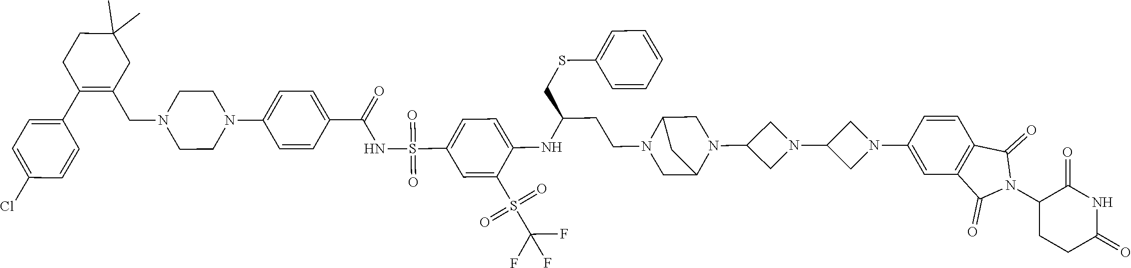 [SMILES: CC1(C)CCC(c2ccc(Cl)cc2)=C(CN2CCN(c3ccc(C(=O)NS(=O)(=O)c4ccc(N[C@H](CCN5CC6CC5CN6C5CN(C6CN(c7ccc8c(c7)C(=O)N(C7CCC(=O)NC7=O)C8=O)C6)C5)CSc5ccccc5)c(S(=O)(=O)C(F)(F)F)c4)cc3)CC2)C1]

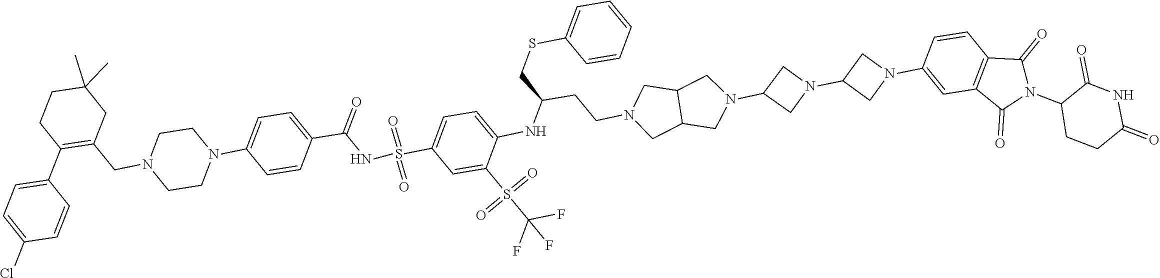 [SMILES: CC1(C)CCC(c2ccc(Cl)cc2)=C(CN2CCN(c3ccc(C(=O)NS(=O)(=O)c4ccc(N[C@H](CCN5CC6CN(C7CN(C8CN(c9ccc%10c(c9)C(=O)N(C9CCC(=O)NC9=O)C%10=O)C8)C7)CC6C5)CSc5ccccc5)c(S(=O)(=O)C(F)(F)F)c4)cc3)CC2)C1]

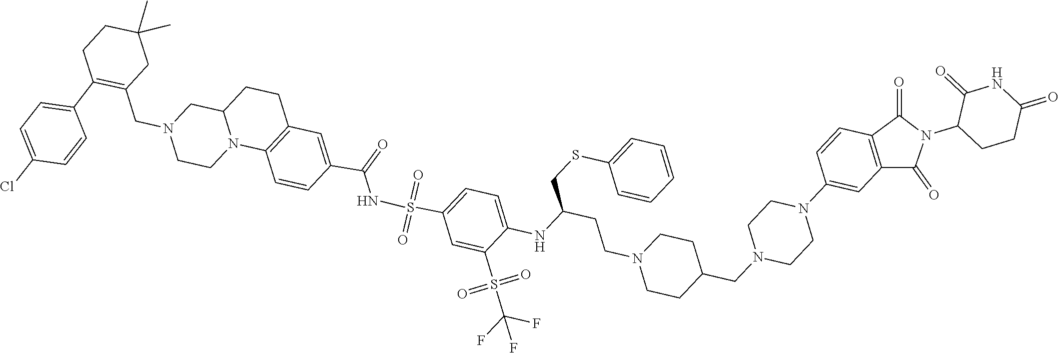 [SMILES: CC1(C)CCC(c2ccc(Cl)cc2)=C(CN2CCN3c4ccc(C(=O)NS(=O)(=O)c5ccc(N[C@H](CCN6CCC(CN7CCN(c8ccc9c(c8)C(=O)N(C8CCC(=O)NC8=O)C9=O)CC7)CC6)CSc6ccccc6)c(S(=O)(=O)C(F)(F)F)c5)cc4CCC3C2)C1]

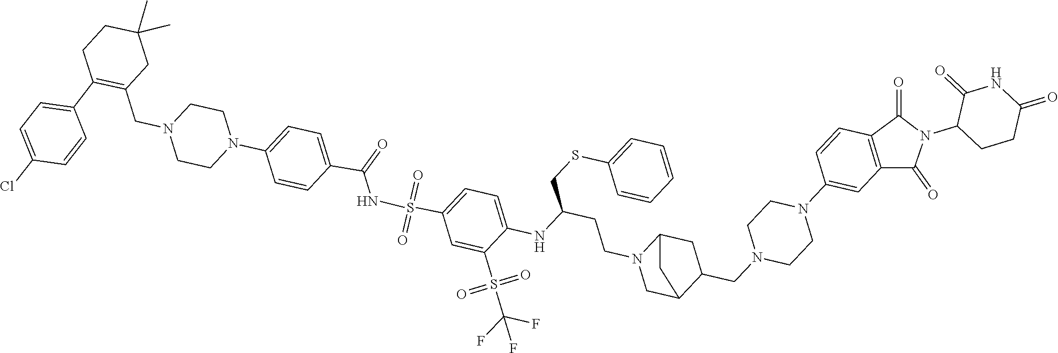 [SMILES: CC1(C)CCC(c2ccc(Cl)cc2)=C(CN2CCN(c3ccc(C(=O)NS(=O)(=O)c4ccc(N[C@H](CCN5CC6CC5CC6CN5CCN(c6ccc7c(c6)C(=O)N(C6CCC(=O)NC6=O)C7=O)CC5)CSc5ccccc5)c(S(=O)(=O)C(F)(F)F)c4)cc3)CC2)C1]

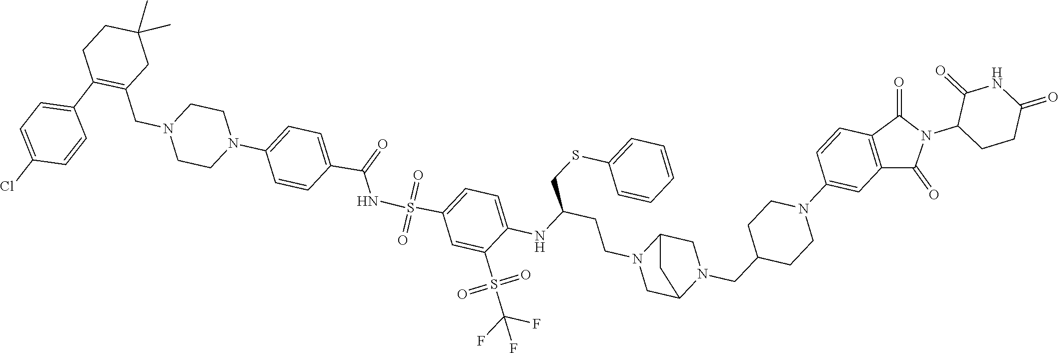 [SMILES: CC1(C)CCC(c2ccc(Cl)cc2)=C(CN2CCN(c3ccc(C(=O)NS(=O)(=O)c4ccc(N[C@H](CCN5CC6CC5CN6CC5CCN(c6ccc7c(c6)C(=O)N(C6CCC(=O)NC6=O)C7=O)CC5)CSc5ccccc5)c(S(=O)(=O)C(F)(F)F)c4)cc3)CC2)C1]

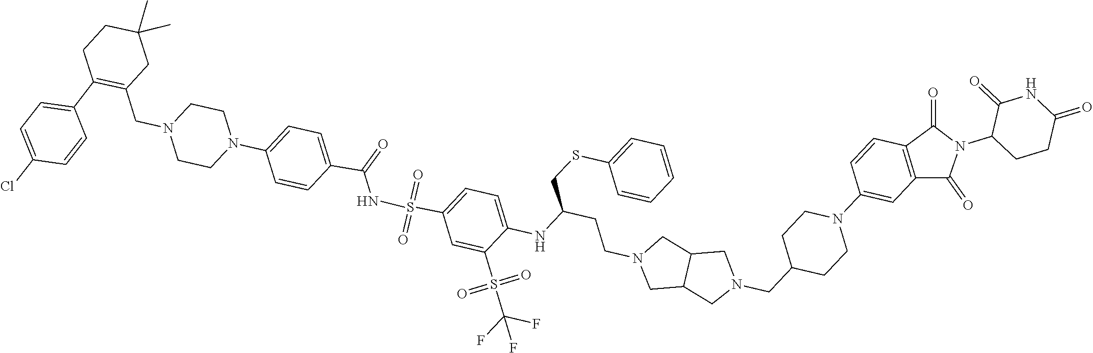 [SMILES: CC1(C)CCC(c2ccc(Cl)cc2)=C(CN2CCN(c3ccc(C(=O)NS(=O)(=O)c4ccc(N[C@H](CCN5CC6CN(CC7CCN(c8ccc9c(c8)C(=O)N(C8CCC(=O)NC8=O)C9=O)CC7)CC6C5)CSc5ccccc5)c(S(=O)(=O)C(F)(F)F)c4)cc3)CC2)C1]

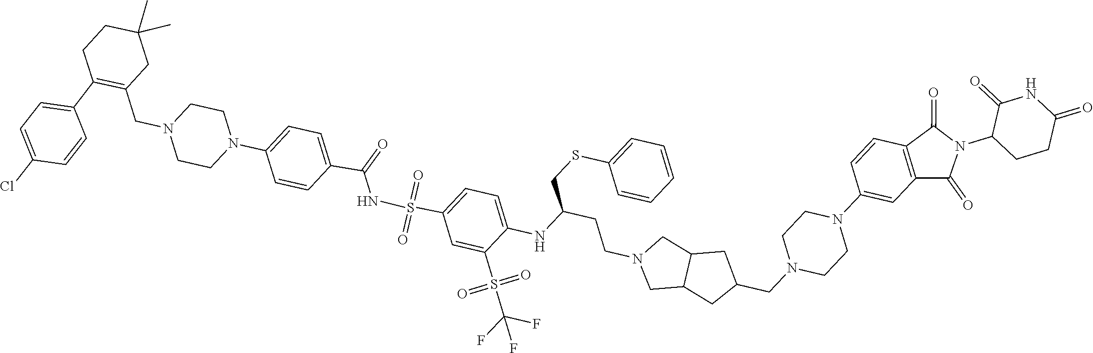 [SMILES: CC1(C)CCC(c2ccc(Cl)cc2)=C(CN2CCN(c3ccc(C(=O)NS(=O)(=O)c4ccc(N[C@H](CCN5CC6CC(CN7CCN(c8ccc9c(c8)C(=O)N(C8CCC(=O)NC8=O)C9=O)CC7)CC6C5)CSc5ccccc5)c(S(=O)(=O)C(F)(F)F)c4)cc3)CC2)C1]

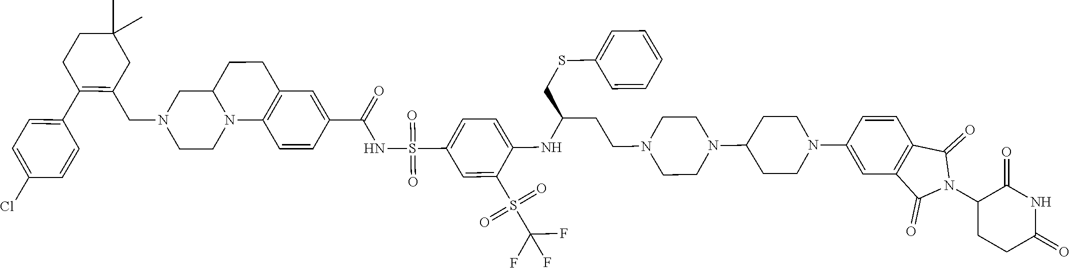 [SMILES: CC1(C)CCC(c2ccc(Cl)cc2)=C(CN2CCN3c4ccc(C(=O)NS(=O)(=O)c5ccc(N[C@H](CCN6CCN(C7CCN(c8ccc9c(c8)C(=O)N(C8CCC(=O)NC8=O)C9=O)CC7)CC6)CSc6ccccc6)c(S(=O)(=O)C(F)(F)F)c5)cc4CCC3C2)C1]

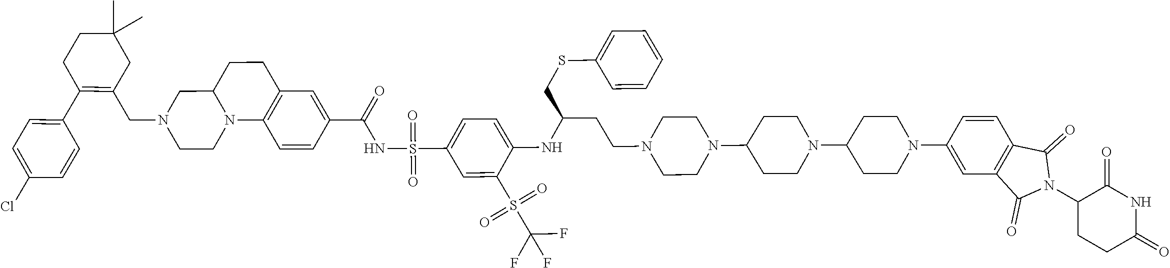 [SMILES: CC1(C)CCC(c2ccc(Cl)cc2)=C(CN2CCN3c4ccc(C(=O)NS(=O)(=O)c5ccc(N[C@H](CCN6CCN(C7CCN(C8CCN(c9ccc%10c(c9)C(=O)N(C9CCC(=O)NC9=O)C%10=O)CC8)CC7)CC6)CSc6ccccc6)c(S(=O)(=O)C(F)(F)F)c5)cc4CCC3C2)C1]

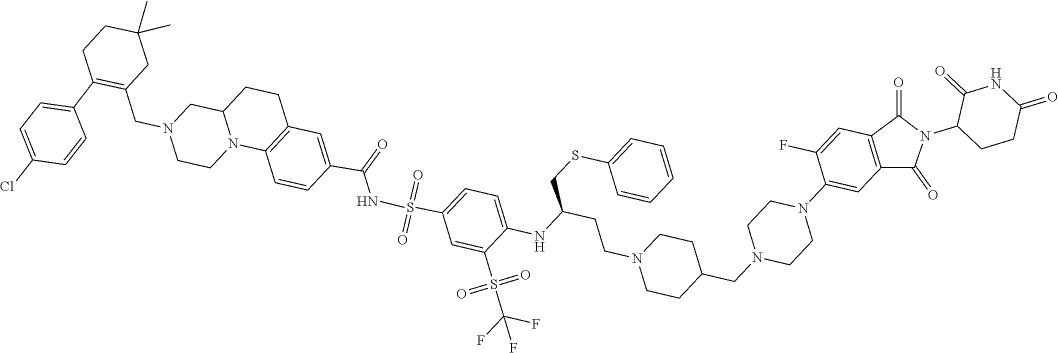 [SMILES: CC1(C)CCC(c2ccc(Cl)cc2)=C(CN2CCN3c4ccc(C(=O)NS(=O)(=O)c5ccc(N[C@H](CCN6CCC(CN7CCN(c8cc9c(cc8F)C(=O)N(C8CCC(=O)NC8=O)C9=O)CC7)CC6)CSc6ccccc6)c(S(=O)(=O)C(F)(F)F)c5)cc4CCC3C2)C1]

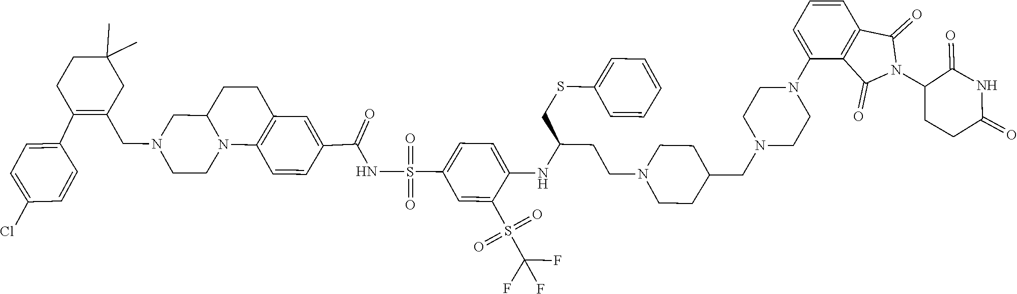 [SMILES: CC1(C)CCC(c2ccc(Cl)cc2)=C(CN2CCN3c4ccc(C(=O)NS(=O)(=O)c5ccc(N[C@H](CCN6CCC(CN7CCN(c8cccc9c8C(=O)N(C8CCC(=O)NC8=O)C9=O)CC7)CC6)CSc6ccccc6)c(S(=O)(=O)C(F)(F)F)c5)cc4CCC3C2)C1]

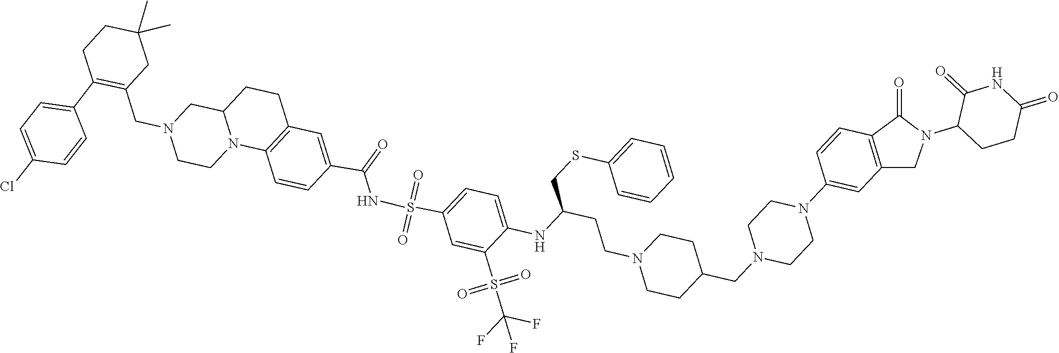 [SMILES: CC1(C)CCC(c2ccc(Cl)cc2)=C(CN2CCN3c4ccc(C(=O)NS(=O)(=O)c5ccc(N[C@H](CCN6CCC(CN7CCN(c8ccc9c(c8)CN(C8CCC(=O)NC8=O)C9=O)CC7)CC6)CSc6ccccc6)c(S(=O)(=O)C(F)(F)F)c5)cc4CCC3C2)C1]

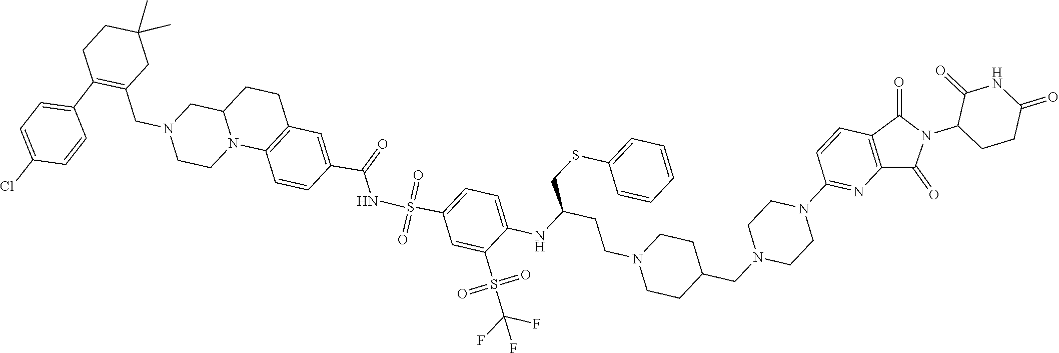 [SMILES: CC1(C)CCC(c2ccc(Cl)cc2)=C(CN2CCN3c4ccc(C(=O)NS(=O)(=O)c5ccc(N[C@H](CCN6CCC(CN7CCN(c8ccc9c(n8)C(=O)N(C8CCC(=O)NC8=O)C9=O)CC7)CC6)CSc6ccccc6)c(S(=O)(=O)C(F)(F)F)c5)cc4CCC3C2)C1]

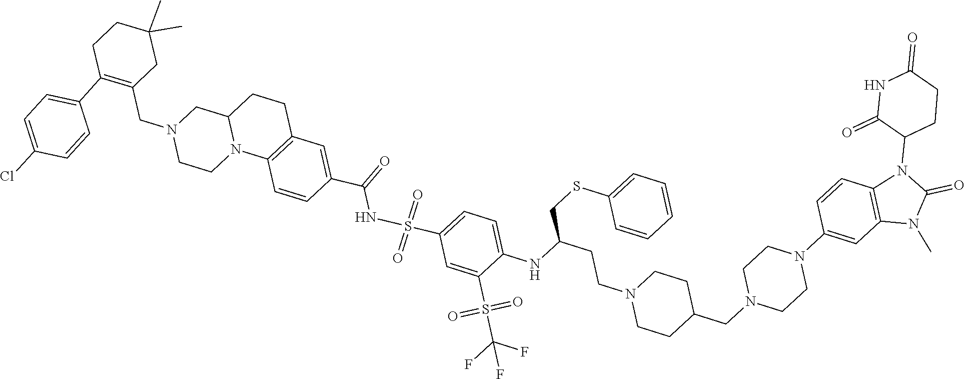 [SMILES: Cn1c(=O)n(C2CCC(=O)NC2=O)c2ccc(N3CCN(CC4CCN(CC[C@H](CSc5ccccc5)Nc5ccc(S(=O)(=O)NC(=O)c6ccc7c(c6)CCC6CN(CC8=C(c9ccc(Cl)cc9)CCC(C)(C)C8)CCN76)cc5S(=O)(=O)C(F)(F)F)CC4)CC3)cc21]

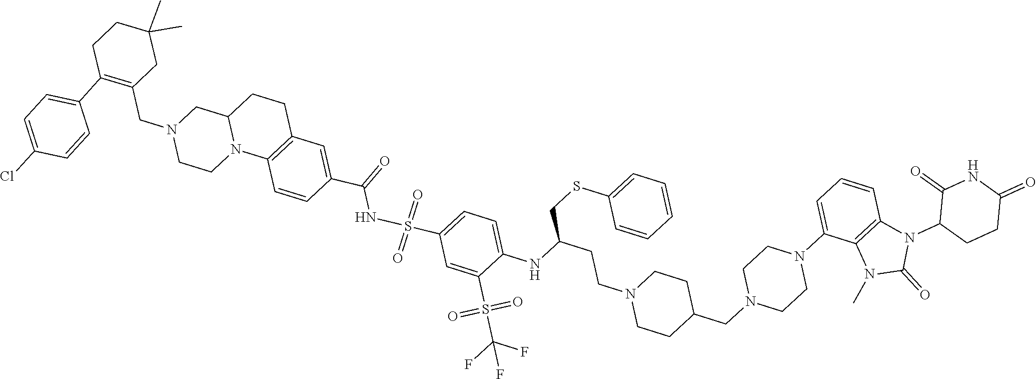 [SMILES: Cn1c(=O)n(C2CCC(=O)NC2=O)c2cccc(N3CCN(CC4CCN(CC[C@H](CSc5ccccc5)Nc5ccc(S(=O)(=O)NC(=O)c6ccc7c(c6)CCC6CN(CC8=C(c9ccc(Cl)cc9)CCC(C)(C)C8)CCN76)cc5S(=O)(=O)C(F)(F)F)CC4)CC3)c21]

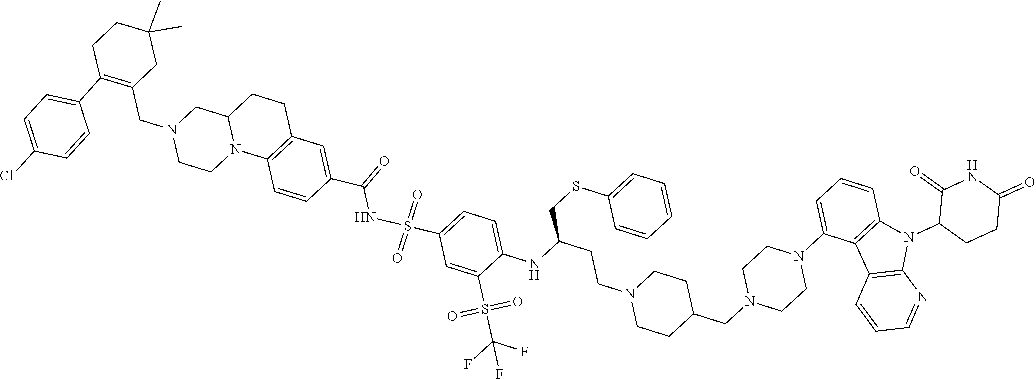 [SMILES: CC1(C)CCC(c2ccc(Cl)cc2)=C(CN2CCN3c4ccc(C(=O)NS(=O)(=O)c5ccc(N[C@H](CCN6CCC(CN7CCN(c8cccc9c8c8cccnc8n9C8CCC(=O)NC8=O)CC7)CC6)CSc6ccccc6)c(S(=O)(=O)C(F)(F)F)c5)cc4CCC3C2)C1]

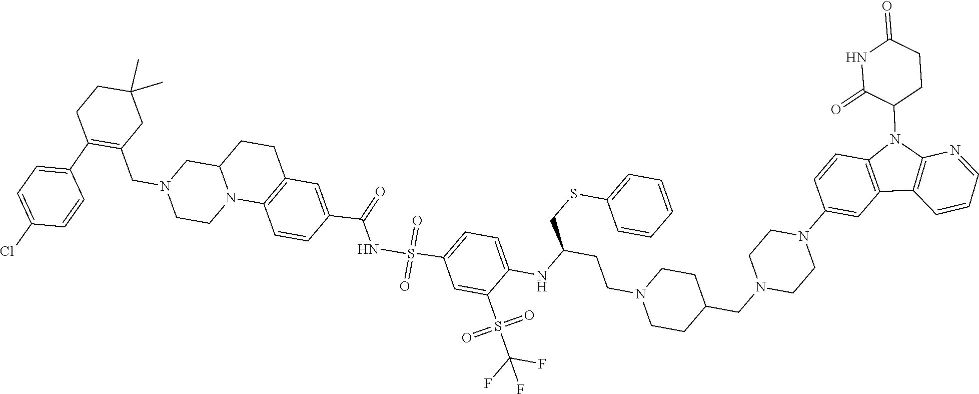 [SMILES: CC1(C)CCC(c2ccc(Cl)cc2)=C(CN2CCN3c4ccc(C(=O)NS(=O)(=O)c5ccc(N[C@H](CCN6CCC(CN7CCN(c8ccc9c(c8)c8cccnc8n9C8CCC(=O)NC8=O)CC7)CC6)CSc6ccccc6)c(S(=O)(=O)C(F)(F)F)c5)cc4CCC3C2)C1]

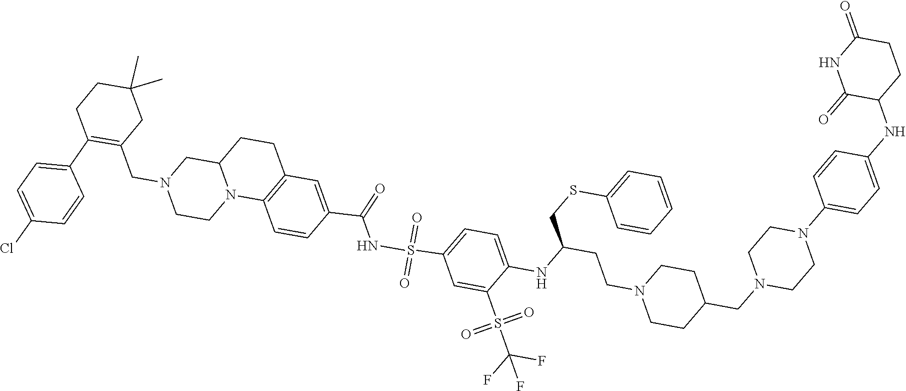 [SMILES: CC1(C)CCC(c2ccc(Cl)cc2)=C(CN2CCN3c4ccc(C(=O)NS(=O)(=O)c5ccc(N[C@H](CCN6CCC(CN7CCN(c8ccc(NC9CCC(=O)NC9=O)cc8)CC7)CC6)CSc6ccccc6)c(S(=O)(=O)C(F)(F)F)c5)cc4CCC3C2)C1]

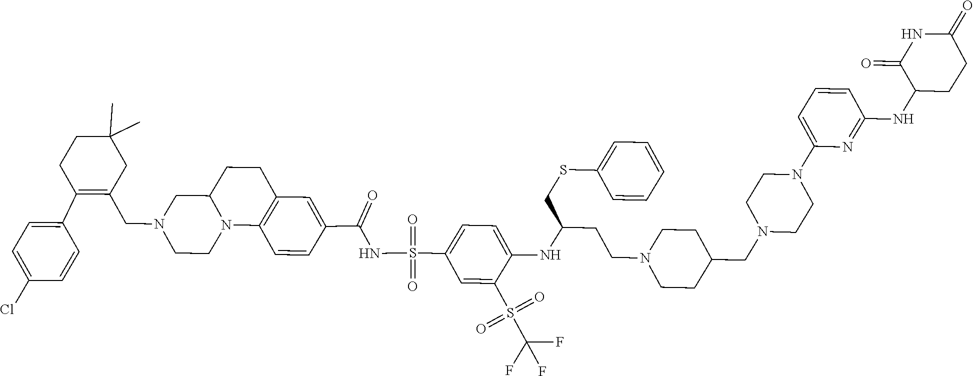 [SMILES: CC1(C)CCC(c2ccc(Cl)cc2)=C(CN2CCN3c4ccc(C(=O)NS(=O)(=O)c5ccc(N[C@H](CCN6CCC(CN7CCN(c8cccc(NC9CCC(=O)NC9=O)n8)CC7)CC6)CSc6ccccc6)c(S(=O)(=O)C(F)(F)F)c5)cc4CCC3C2)C1]

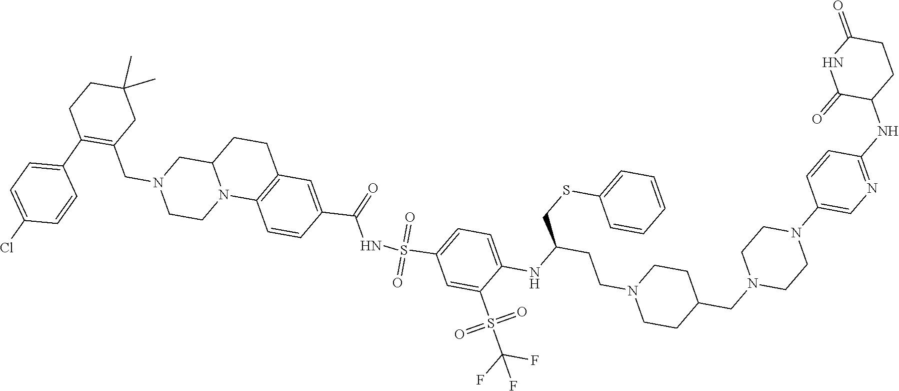 [SMILES: CC1(C)CCC(c2ccc(Cl)cc2)=C(CN2CCN3c4ccc(C(=O)NS(=O)(=O)c5ccc(N[C@H](CCN6CCC(CN7CCN(c8ccc(NC9CCC(=O)NC9=O)nc8)CC7)CC6)CSc6ccccc6)c(S(=O)(=O)C(F)(F)F)c5)cc4CCC3C2)C1]

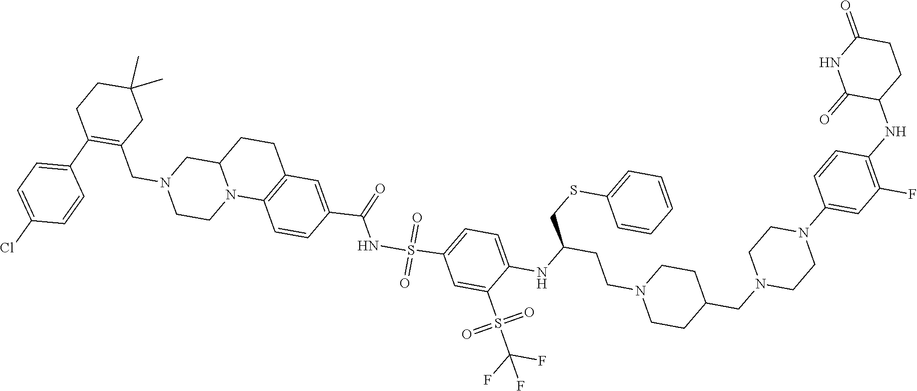 [SMILES: CC1(C)CCC(c2ccc(Cl)cc2)=C(CN2CCN3c4ccc(C(=O)NS(=O)(=O)c5ccc(N[C@H](CCN6CCC(CN7CCN(c8ccc(NC9CCC(=O)NC9=O)c(F)c8)CC7)CC6)CSc6ccccc6)c(S(=O)(=O)C(F)(F)F)c5)cc4CCC3C2)C1]

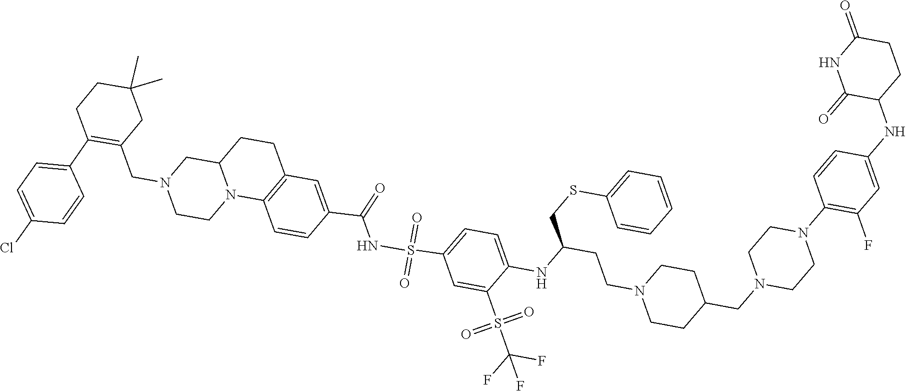 [SMILES: CC1(C)CCC(c2ccc(Cl)cc2)=C(CN2CCN3c4ccc(C(=O)NS(=O)(=O)c5ccc(N[C@H](CCN6CCC(CN7CCN(c8ccc(NC9CCC(=O)NC9=O)cc8F)CC7)CC6)CSc6ccccc6)c(S(=O)(=O)C(F)(F)F)c5)cc4CCC3C2)C1]

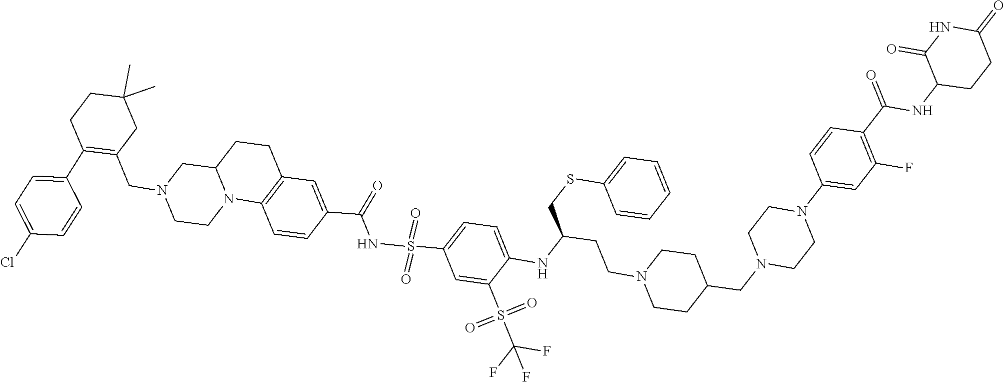 [SMILES: CC1(C)CCC(c2ccc(Cl)cc2)=C(CN2CCN3c4ccc(C(=O)NS(=O)(=O)c5ccc(N[C@H](CCN6CCC(CN7CCN(c8ccc(C(=O)NC9CCC(=O)NC9=O)c(F)c8)CC7)CC6)CSc6ccccc6)c(S(=O)(=O)C(F)(F)F)c5)cc4CCC3C2)C1]

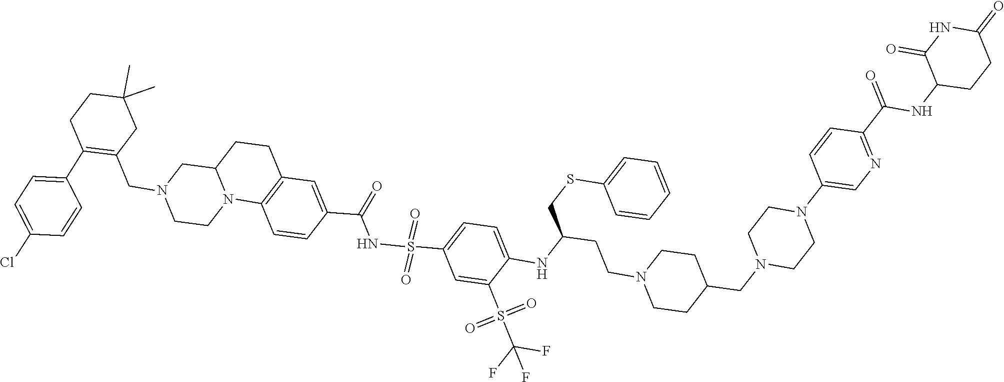 [SMILES: CC1(C)CCC(c2ccc(Cl)cc2)=C(CN2CCN3c4ccc(C(=O)NS(=O)(=O)c5ccc(N[C@H](CCN6CCC(CN7CCN(c8ccc(C(=O)NC9CCC(=O)NC9=O)nc8)CC7)CC6)CSc6ccccc6)c(S(=O)(=O)C(F)(F)F)c5)cc4CCC3C2)C1]

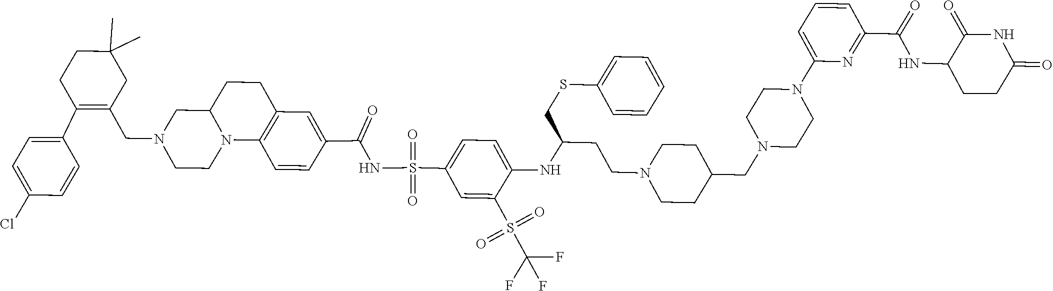 [SMILES: CC1(C)CCC(c2ccc(Cl)cc2)=C(CN2CCN3c4ccc(C(=O)NS(=O)(=O)c5ccc(N[C@H](CCN6CCC(CN7CCN(c8cccc(C(=O)NC9CCC(=O)NC9=O)n8)CC7)CC6)CSc6ccccc6)c(S(=O)(=O)C(F)(F)F)c5)cc4CCC3C2)C1]

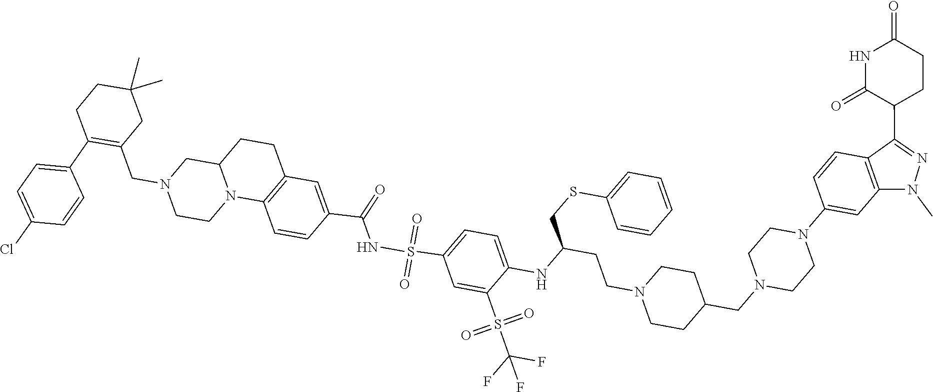 [SMILES: Cn1nc(C2CCC(=O)NC2=O)c2ccc(N3CCN(CC4CCN(CC[C@H](CSc5ccccc5)Nc5ccc(S(=O)(=O)NC(=O)c6ccc7c(c6)CCC6CN(CC8=C(c9ccc(Cl)cc9)CCC(C)(C)C8)CCN76)cc5S(=O)(=O)C(F)(F)F)CC4)CC3)cc21]

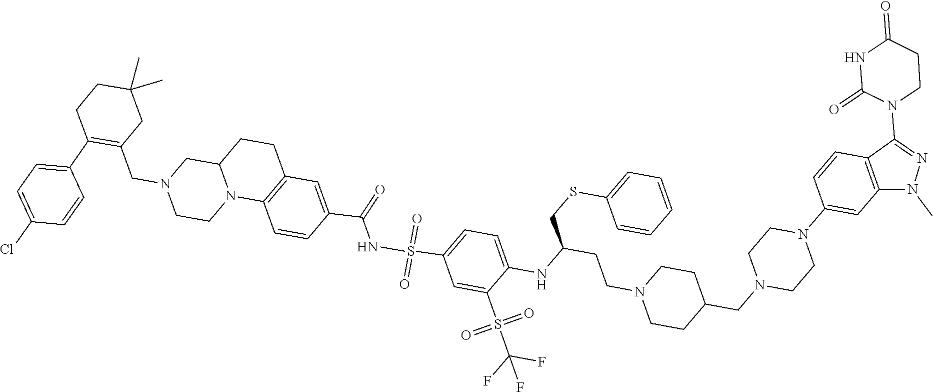 [SMILES: Cn1nc(N2CCC(=O)NC2=O)c2ccc(N3CCN(CC4CCN(CC[C@H](CSc5ccccc5)Nc5ccc(S(=O)(=O)NC(=O)c6ccc7c(c6)CCC6CN(CC8=C(c9ccc(Cl)cc9)CCC(C)(C)C8)CCN76)cc5S(=O)(=O)C(F)(F)F)CC4)CC3)cc21]

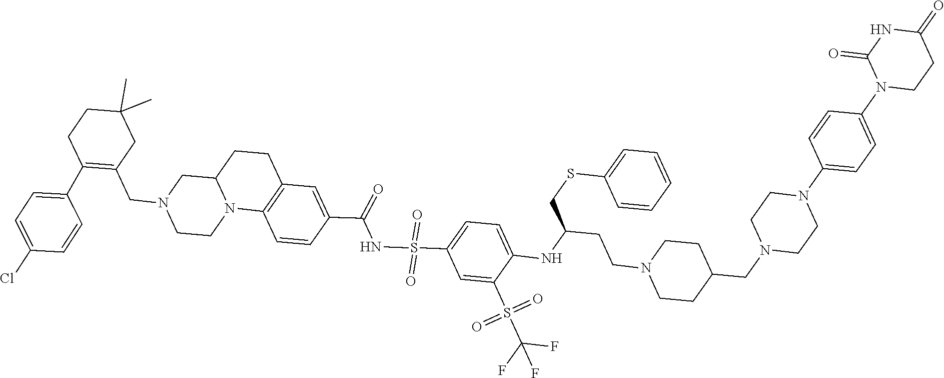 [SMILES: CC1(C)CCC(c2ccc(Cl)cc2)=C(CN2CCN3c4ccc(C(=O)NS(=O)(=O)c5ccc(N[C@H](CCN6CCC(CN7CCN(c8ccc(N9CCC(=O)NC9=O)cc8)CC7)CC6)CSc6ccccc6)c(S(=O)(=O)C(F)(F)F)c5)cc4CCC3C2)C1]

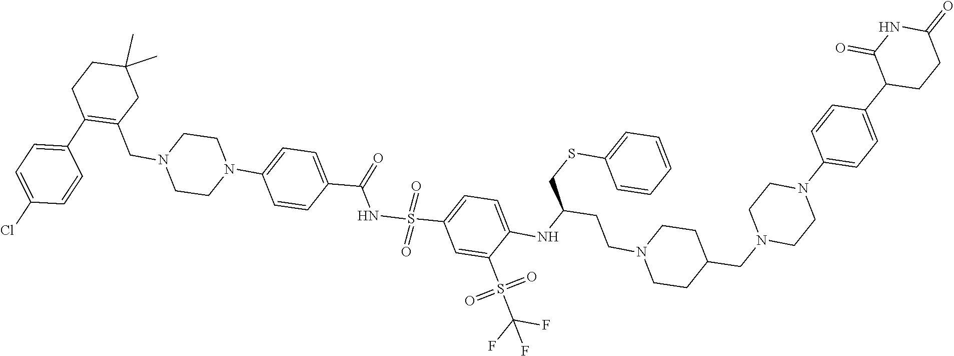 [SMILES: CC1(C)CCC(c2ccc(Cl)cc2)=C(CN2CCN(c3ccc(C(=O)NS(=O)(=O)c4ccc(N[C@H](CCN5CCC(CN6CCN(c7ccc(C8CCC(=O)NC8=O)cc7)CC6)CC5)CSc5ccccc5)c(S(=O)(=O)C(F)(F)F)c4)cc3)CC2)C1]

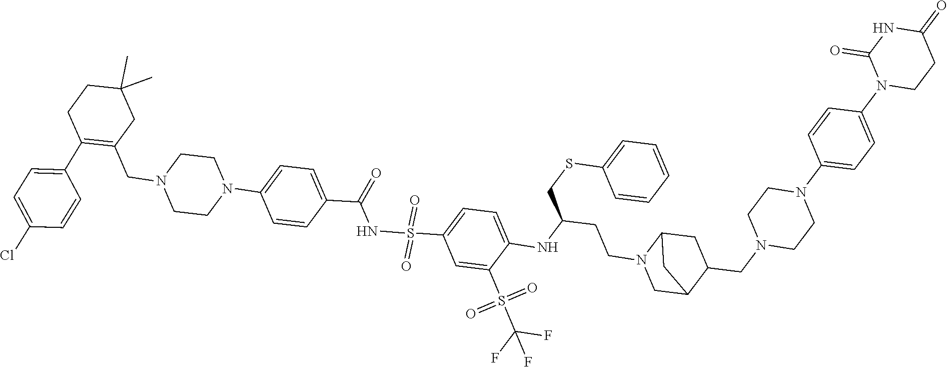 [SMILES: CC1(C)CCC(c2ccc(Cl)cc2)=C(CN2CCN(c3ccc(C(=O)NS(=O)(=O)c4ccc(N[C@H](CCN5CC6CC5CC6CN5CCN(c6ccc(N7CCC(=O)NC7=O)cc6)CC5)CSc5ccccc5)c(S(=O)(=O)C(F)(F)F)c4)cc3)CC2)C1]

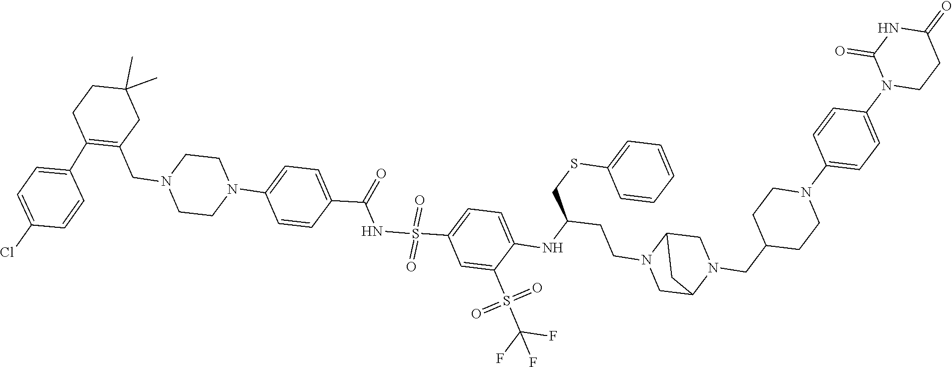 [SMILES: CC1(C)CCC(c2ccc(Cl)cc2)=C(CN2CCN(c3ccc(C(=O)NS(=O)(=O)c4ccc(N[C@H](CCN5CC6CC5CN6CC5CCN(c6ccc(N7CCC(=O)NC7=O)cc6)CC5)CSc5ccccc5)c(S(=O)(=O)C(F)(F)F)c4)cc3)CC2)C1]

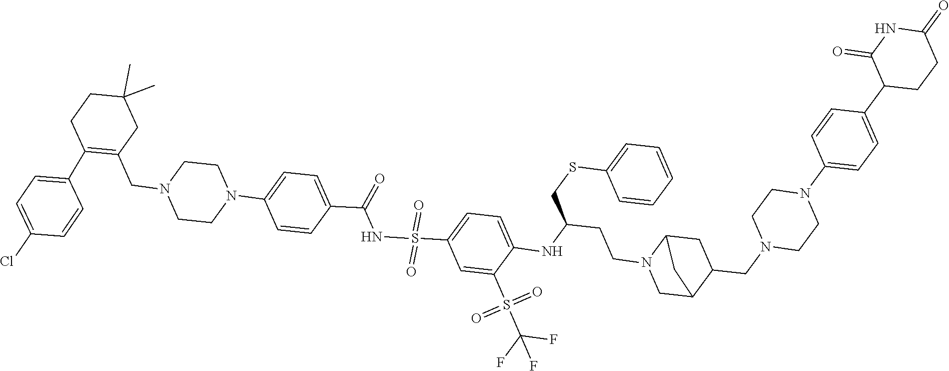 [SMILES: CC1(C)CCC(c2ccc(Cl)cc2)=C(CN2CCN(c3ccc(C(=O)NS(=O)(=O)c4ccc(N[C@H](CCN5CC6CC5CC6CN5CCN(c6ccc(C7CCC(=O)NC7=O)cc6)CC5)CSc5ccccc5)c(S(=O)(=O)C(F)(F)F)c4)cc3)CC2)C1]

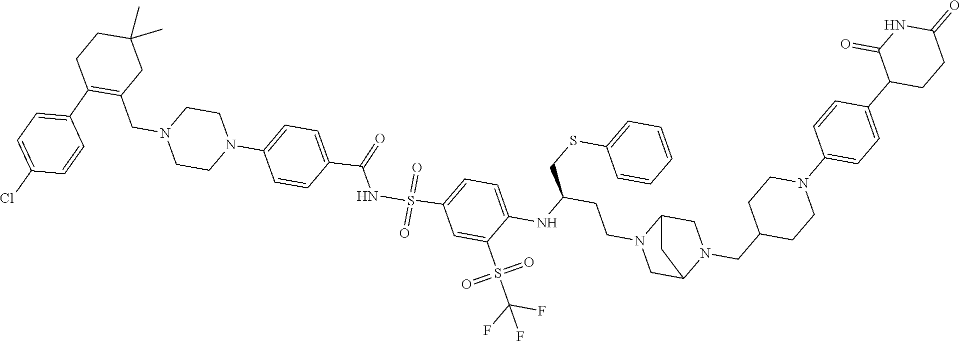 [SMILES: CC1(C)CCC(c2ccc(Cl)cc2)=C(CN2CCN(c3ccc(C(=O)NS(=O)(=O)c4ccc(N[C@H](CCN5CC6CC5CN6CC5CCN(c6ccc(C7CCC(=O)NC7=O)cc6)CC5)CSc5ccccc5)c(S(=O)(=O)C(F)(F)F)c4)cc3)CC2)C1]

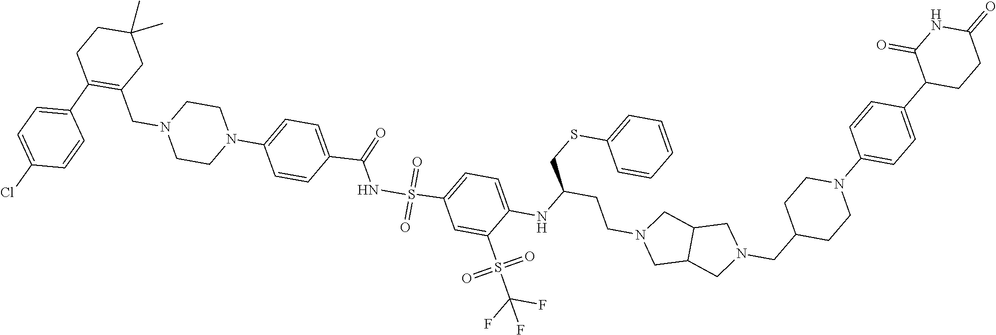 [SMILES: CC1(C)CCC(c2ccc(Cl)cc2)=C(CN2CCN(c3ccc(C(=O)NS(=O)(=O)c4ccc(N[C@H](CCN5CC6CN(CC7CCN(c8ccc(C9CCC(=O)NC9=O)cc8)CC7)CC6C5)CSc5ccccc5)c(S(=O)(=O)C(F)(F)F)c4)cc3)CC2)C1]

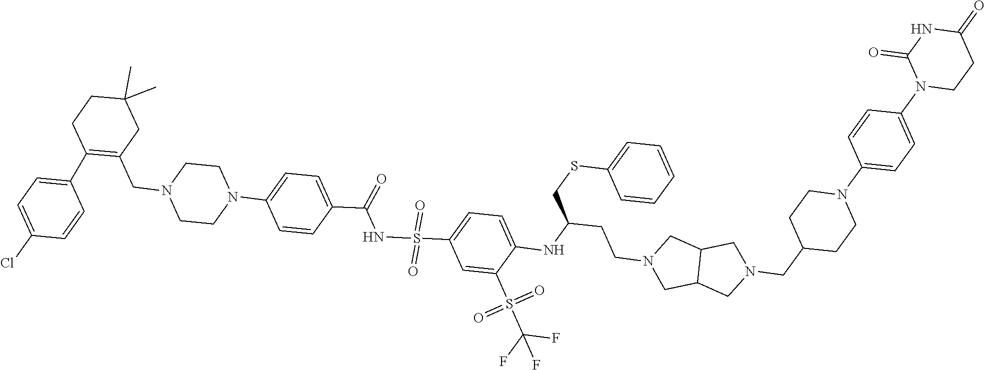 [SMILES: CC1(C)CCC(c2ccc(Cl)cc2)=C(CN2CCN(c3ccc(C(=O)NS(=O)(=O)c4ccc(N[C@H](CCN5CC6CN(CC7CCN(c8ccc(N9CCC(=O)NC9=O)cc8)CC7)CC6C5)CSc5ccccc5)c(S(=O)(=O)C(F)(F)F)c4)cc3)CC2)C1]

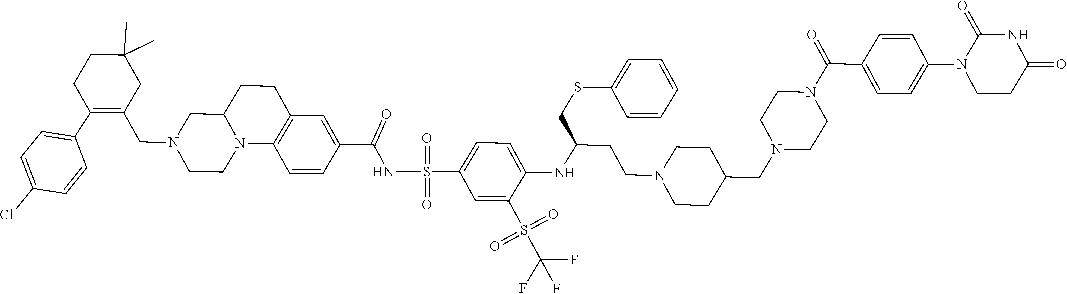 [SMILES: CC1(C)CCC(c2ccc(Cl)cc2)=C(CN2CCN3c4ccc(C(=O)NS(=O)(=O)c5ccc(N[C@H](CCN6CCC(CN7CCN(C(=O)c8ccc(N9CCC(=O)NC9=O)cc8)CC7)CC6)CSc6ccccc6)c(S(=O)(=O)C(F)(F)F)c5)cc4CCC3C2)C1]

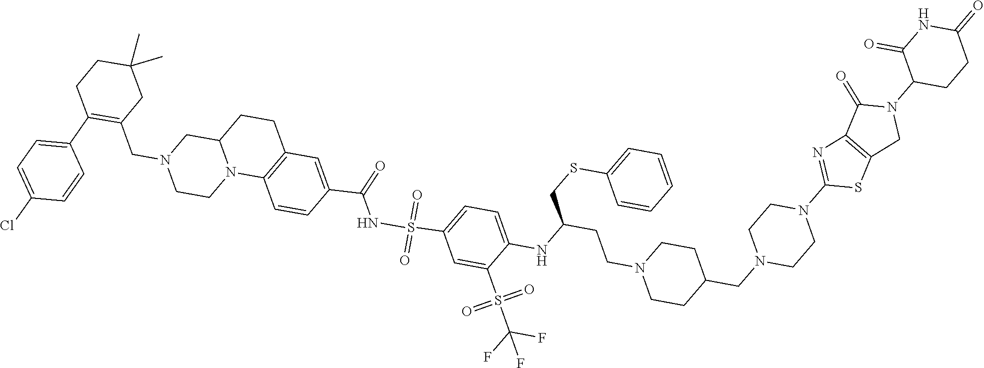 [SMILES: CC1(C)CCC(c2ccc(Cl)cc2)=C(CN2CCN3c4ccc(C(=O)NS(=O)(=O)c5ccc(N[C@H](CCN6CCC(CN7CCN(c8nc9c(s8)CN(C8CCC(=O)NC8=O)C9=O)CC7)CC6)CSc6ccccc6)c(S(=O)(=O)C(F)(F)F)c5)cc4CCC3C2)C1]

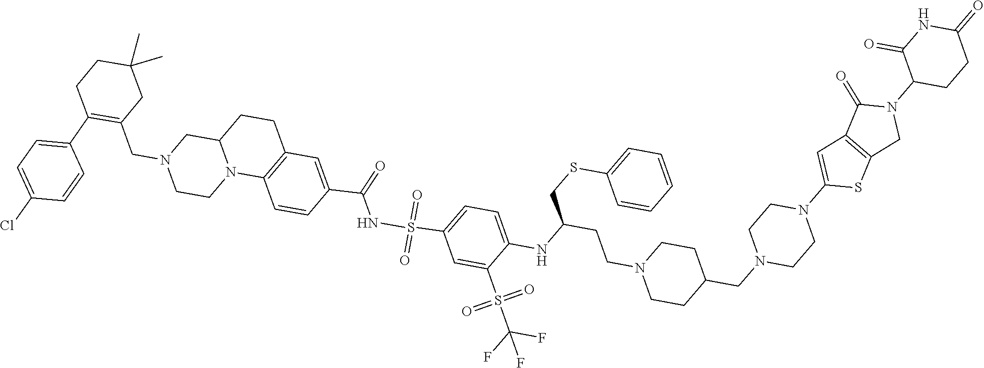 [SMILES: CC1(C)CCC(c2ccc(Cl)cc2)=C(CN2CCN3c4ccc(C(=O)NS(=O)(=O)c5ccc(N[C@H](CCN6CCC(CN7CCN(c8cc9c(s8)CN(C8CCC(=O)NC8=O)C9=O)CC7)CC6)CSc6ccccc6)c(S(=O)(=O)C(F)(F)F)c5)cc4CCC3C2)C1]

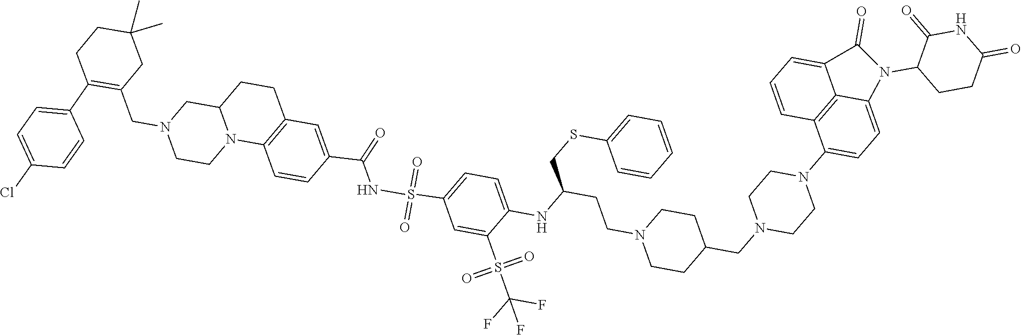 [SMILES: CC1(C)CCC(c2ccc(Cl)cc2)=C(CN2CCN3c4ccc(C(=O)NS(=O)(=O)c5ccc(N[C@H](CCN6CCC(CN7CCN(c8ccc9c%10c(cccc8%10)C(=O)N9C8CCC(=O)NC8=O)CC7)CC6)CSc6ccccc6)c(S(=O)(=O)C(F)(F)F)c5)cc4CCC3C2)C1]

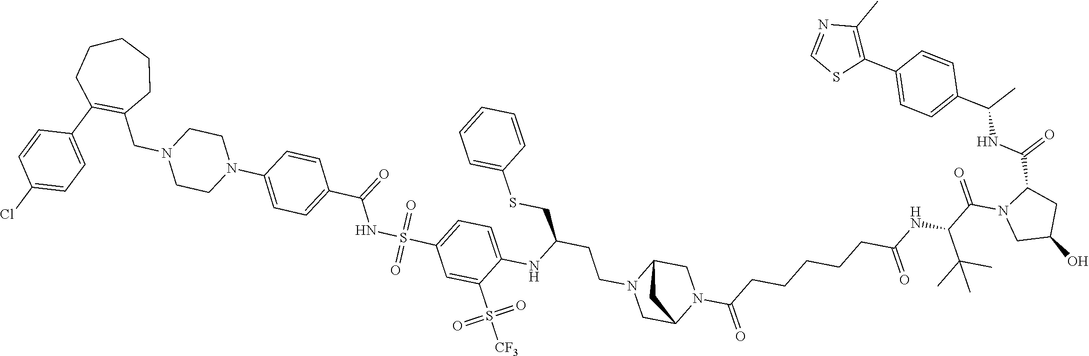 [SMILES: Cc1ncsc1-c1ccc([C@H](C)NC(=O)[C@@H]2C[C@@H](O)CN2C(=O)[C@@H](NC(=O)CCCCCC(=O)N2C[C@@H]3C[C@H]2CN3CC[C@H](CSc2ccccc2)Nc2ccc(S(=O)(=O)NC(=O)c3ccc(N4CCN(CC5=C(c6ccc(Cl)cc6)CCCCC5)CC4)cc3)cc2S(=O)(=O)C(F)(F)F)C(C)(C)C)cc1]

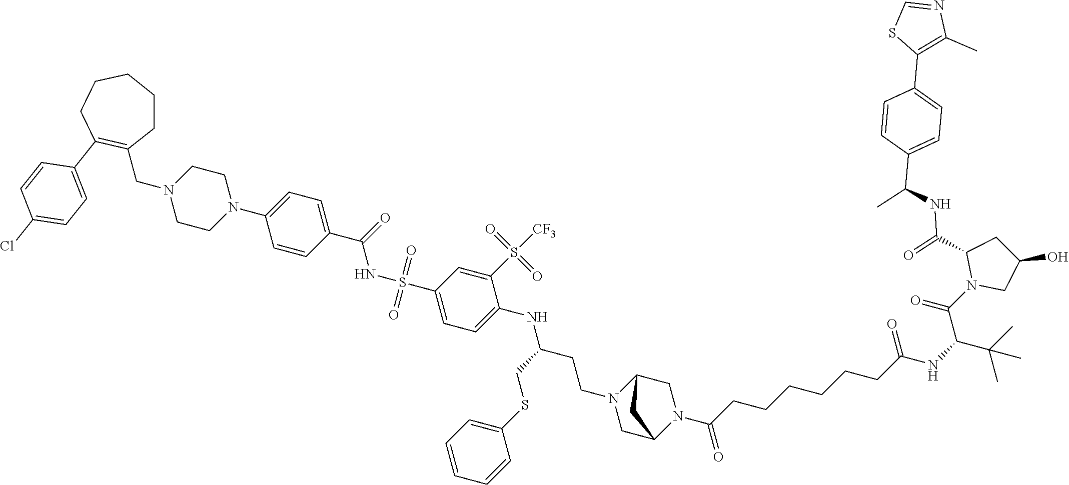 [SMILES: Cc1ncsc1-c1ccc([C@H](C)NC(=O)[C@@H]2C[C@@H](O)CN2C(=O)[C@@H](NC(=O)CCCCCCC(=O)N2C[C@@H]3C[C@H]2CN3CC[C@H](CSc2ccccc2)Nc2ccc(S(=O)(=O)NC(=O)c3ccc(N4CCN(CC5=C(c6ccc(Cl)cc6)CCCCC5)CC4)cc3)cc2S(=O)(=O)C(F)(F)F)C(C)(C)C)cc1]

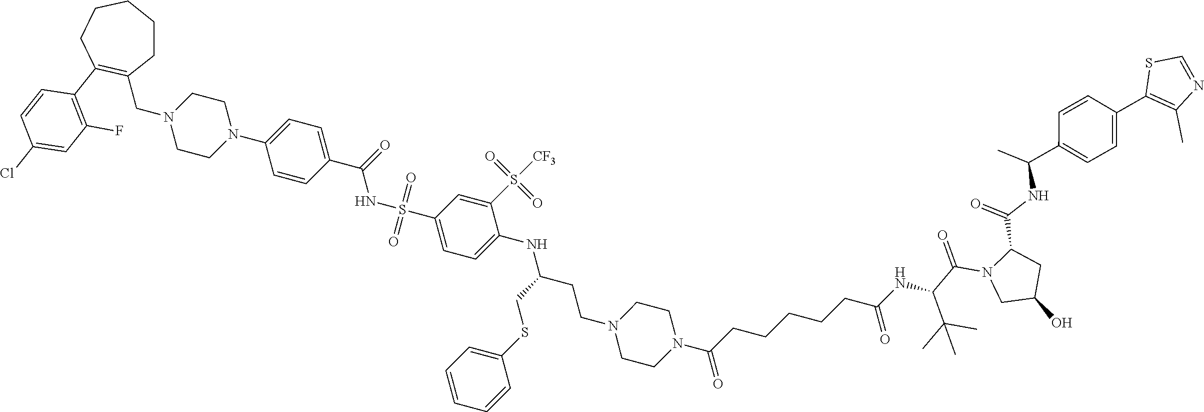 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7F)CCCCC6)CC5)cc4)cc3S(=O)(=O)C(F)(F)F)CC2)C(C)(C)C)cc1]

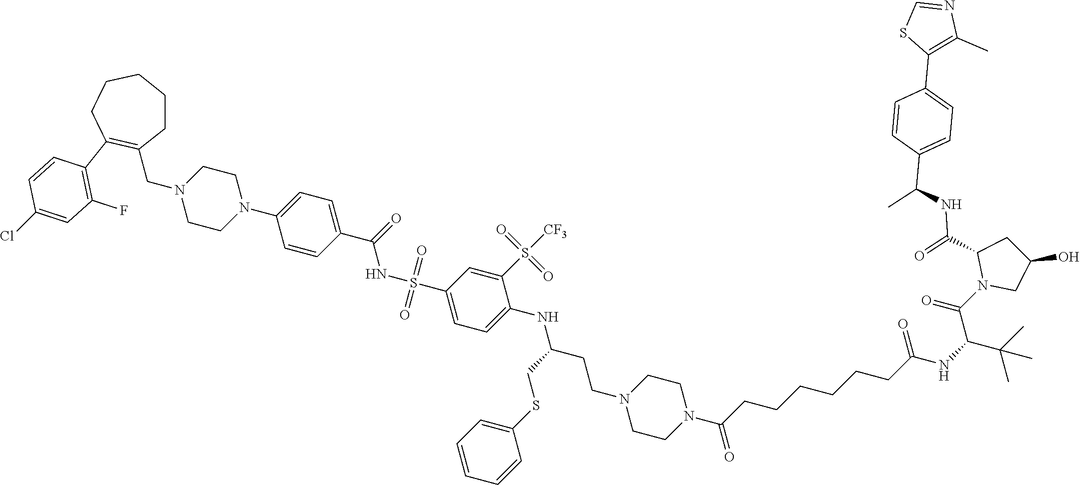 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC[C@H](CSc3ccccc3)Nc3ccc(S(=O)(=O)NC(=O)c4ccc(N5CCN(CC6=C(c7ccc(Cl)cc7F)CCCCC6)CC5)cc4)cc3S(=O)(=O)C(F)(F)F)CC2)C(C)(C)C)cc1]

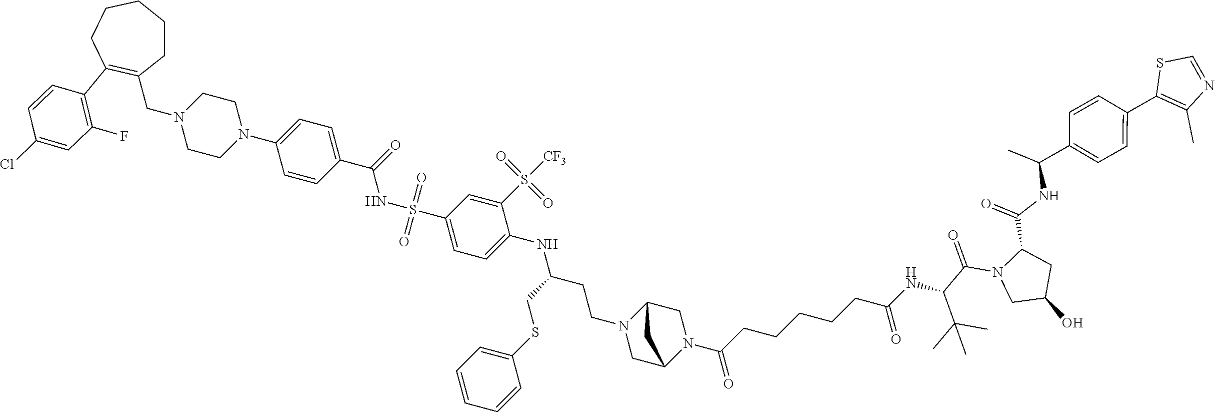 [SMILES: Cc1ncsc1-c1ccc([C@H](C)NC(=O)[C@@H]2C[C@@H](O)CN2C(=O)[C@@H](NC(=O)CCCCCC(=O)N2C[C@@H]3C[C@H]2CN3CC[C@H](CSc2ccccc2)Nc2ccc(S(=O)(=O)NC(=O)c3ccc(N4CCN(CC5=C(c6ccc(Cl)cc6F)CCCCC5)CC4)cc3)cc2S(=O)(=O)C(F)(F)F)C(C)(C)C)cc1]

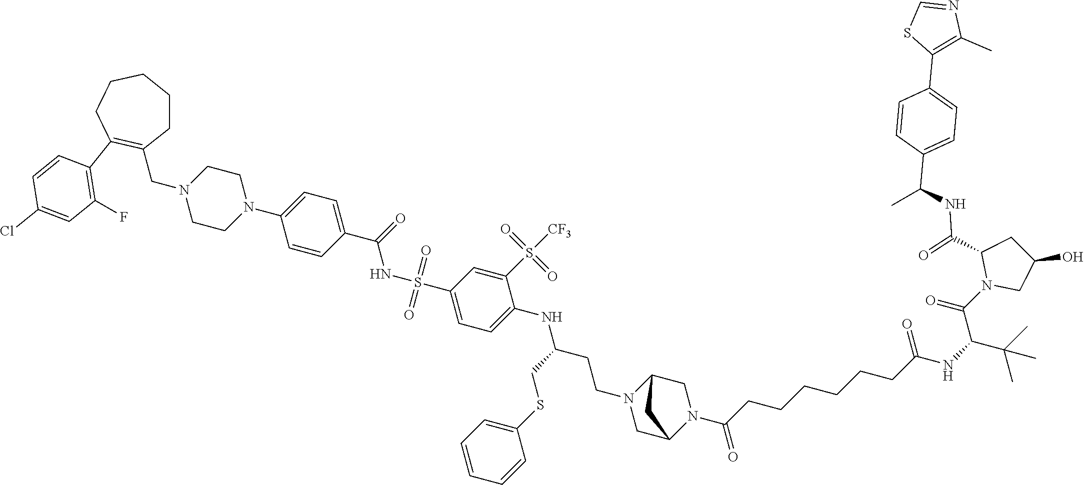 [SMILES: Cc1ncsc1-c1ccc([C@H](C)NC(=O)[C@@H]2C[C@@H](O)CN2C(=O)[C@@H](NC(=O)CCCCCCC(=O)N2C[C@@H]3C[C@H]2CN3CC[C@H](CSc2ccccc2)Nc2ccc(S(=O)(=O)NC(=O)c3ccc(N4CCN(CC5=C(c6ccc(Cl)cc6F)CCCCC5)CC4)cc3)cc2S(=O)(=O)C(F)(F)F)C(C)(C)C)cc1]

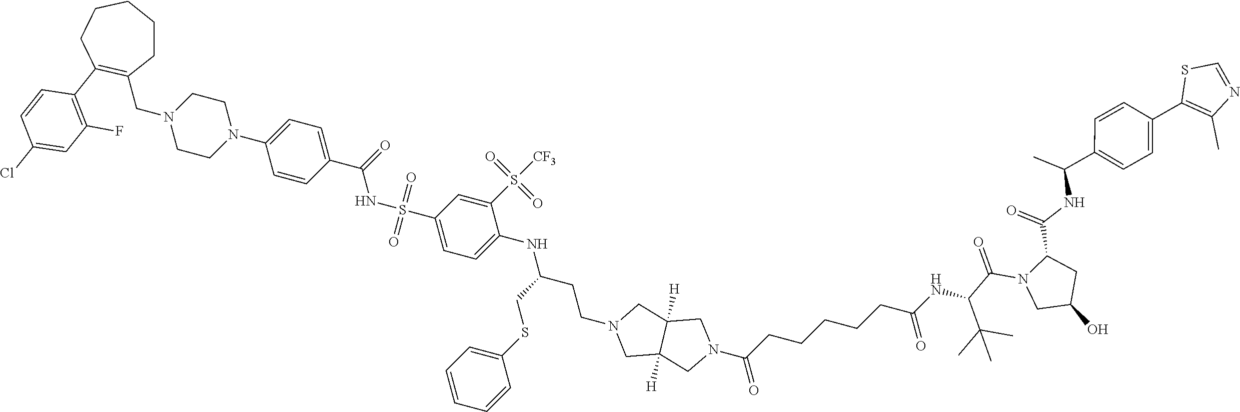 [SMILES: Cc1ncsc1-c1ccc([C@H](C)NC(=O)[C@@H]2C[C@@H](O)CN2C(=O)[C@@H](NC(=O)CCCCCC(=O)N2C[C@H]3CN(CC[C@H](CSc4ccccc4)Nc4ccc(S(=O)(=O)NC(=O)c5ccc(N6CCN(CC7=C(c8ccc(Cl)cc8F)CCCCC7)CC6)cc5)cc4S(=O)(=O)C(F)(F)F)C[C@H]3C2)C(C)(C)C)cc1]

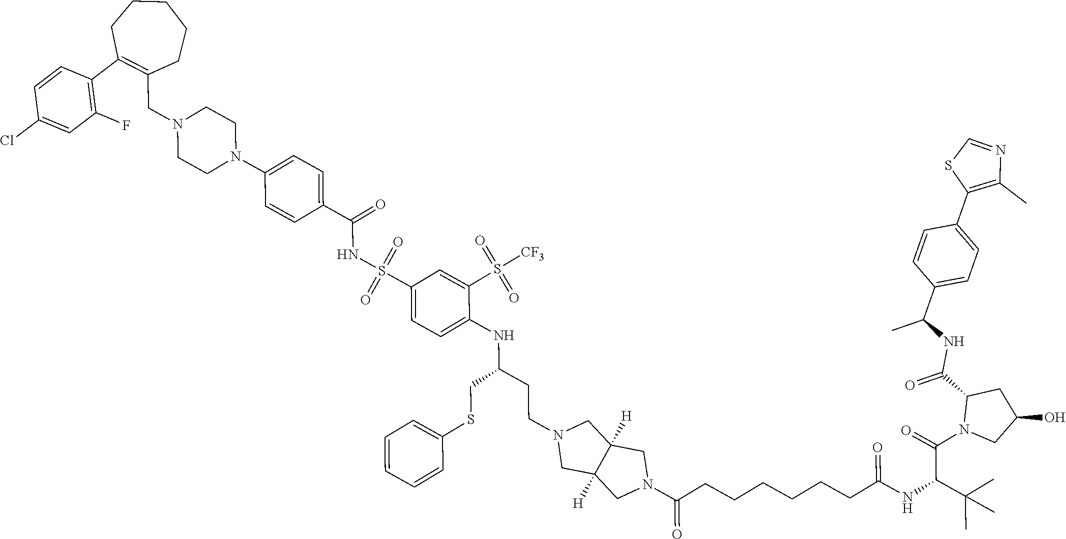 [SMILES: Cc1ncsc1-c1ccc([C@H](C)NC(=O)[C@@H]2C[C@@H](O)CN2C(=O)[C@@H](NC(=O)CCCCCCC(=O)N2C[C@H]3CN(CC[C@H](CSc4ccccc4)Nc4ccc(S(=O)(=O)NC(=O)c5ccc(N6CCN(CC7=C(c8ccc(Cl)cc8F)CCCCC7)CC6)cc5)cc4S(=O)(=O)C(F)(F)F)C[C@H]3C2)C(C)(C)C)cc1]

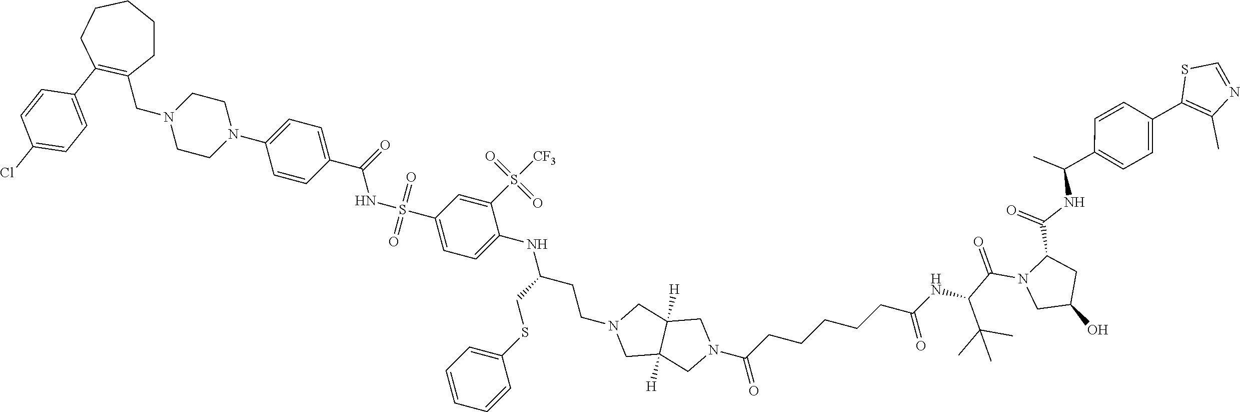 [SMILES: Cc1ncsc1-c1ccc([C@H](C)NC(=O)[C@@H]2C[C@@H](O)CN2C(=O)[C@@H](NC(=O)CCCCCC(=O)N2C[C@H]3CN(CC[C@H](CSc4ccccc4)Nc4ccc(S(=O)(=O)NC(=O)c5ccc(N6CCN(CC7=C(c8ccc(Cl)cc8)CCCCC7)CC6)cc5)cc4S(=O)(=O)C(F)(F)F)C[C@H]3C2)C(C)(C)C)cc1]

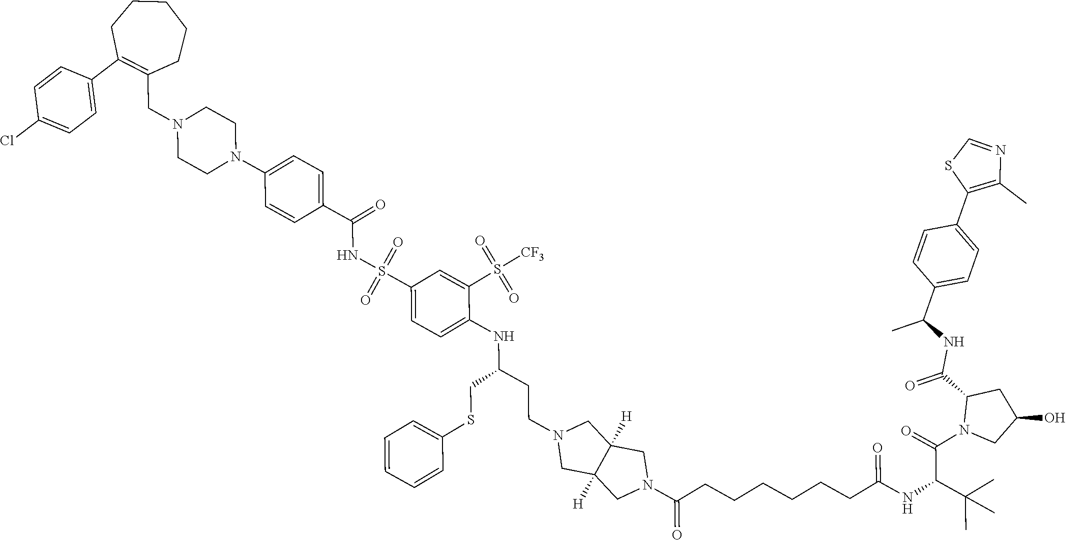 [SMILES: Cc1ncsc1-c1ccc([C@H](C)NC(=O)[C@@H]2C[C@@H](O)CN2C(=O)[C@@H](NC(=O)CCCCCCC(=O)N2C[C@H]3CN(CC[C@H](CSc4ccccc4)Nc4ccc(S(=O)(=O)NC(=O)c5ccc(N6CCN(CC7=C(c8ccc(Cl)cc8)CCCCC7)CC6)cc5)cc4S(=O)(=O)C(F)(F)F)C[C@H]3C2)C(C)(C)C)cc1]

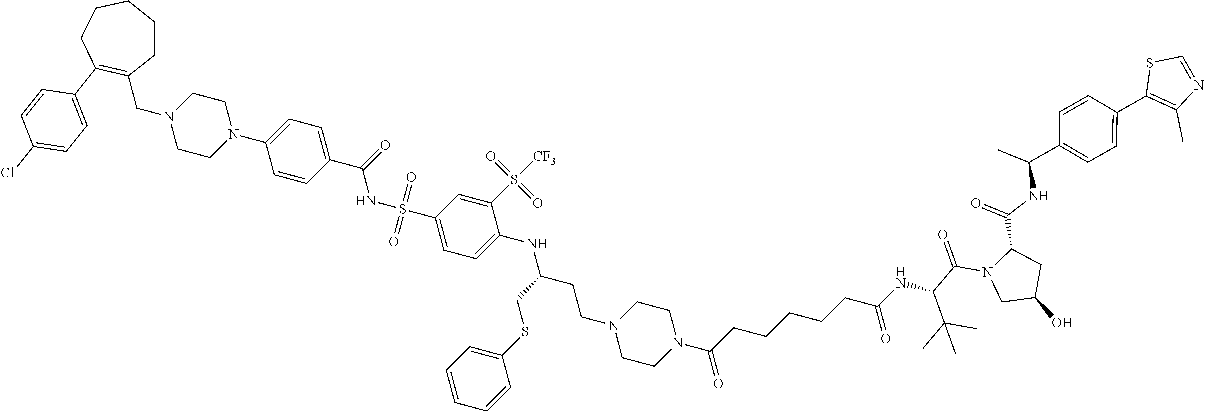 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCCCC6)CC5)cc4)cc3S(=O)(=O)C(F)(F)F)CC2)C(C)(C)C)cc1]

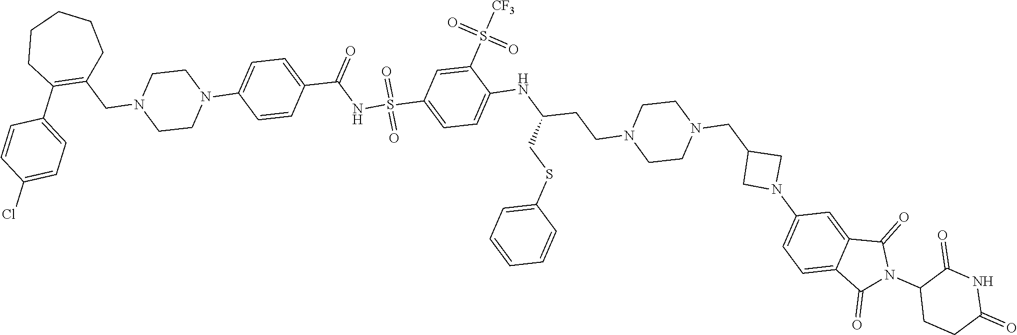 [SMILES: O=C1CCC(N2C(=O)c3ccc(N4CC(CN5CCN(CC[C@H](CSc6ccccc6)Nc6ccc(S(=O)(=O)NC(=O)c7ccc(N8CCN(CC9=C(c%10ccc(Cl)cc%10)CCCCC9)CC8)cc7)cc6S(=O)(=O)C(F)(F)F)CC5)C4)cc3C2=O)C(=O)N1]

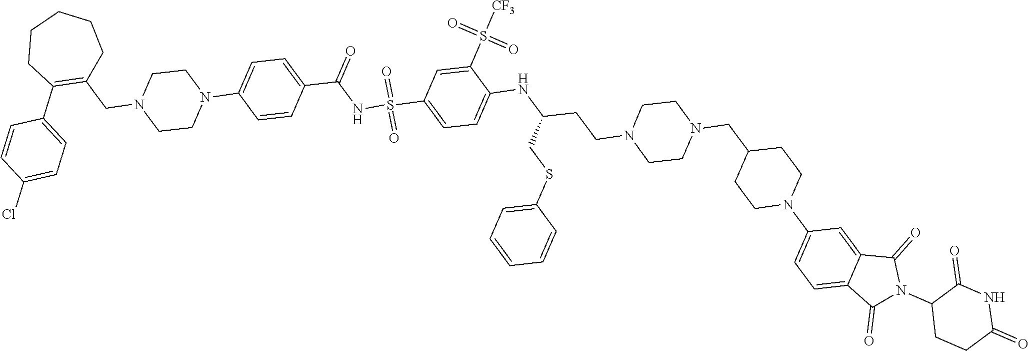 [SMILES: O=C1CCC(N2C(=O)c3ccc(N4CCC(CN5CCN(CC[C@H](CSc6ccccc6)Nc6ccc(S(=O)(=O)NC(=O)c7ccc(N8CCN(CC9=C(c%10ccc(Cl)cc%10)CCCCC9)CC8)cc7)cc6S(=O)(=O)C(F)(F)F)CC5)CC4)cc3C2=O)C(=O)N1]

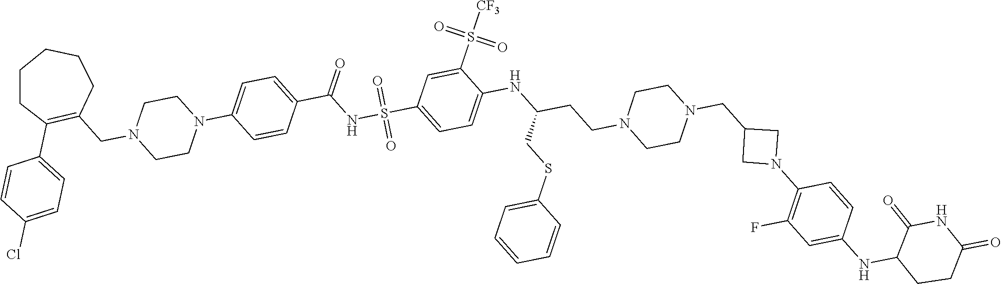 [SMILES: O=C1CCC(Nc2ccc(N3CC(CN4CCN(CC[C@H](CSc5ccccc5)Nc5ccc(S(=O)(=O)NC(=O)c6ccc(N7CCN(CC8=C(c9ccc(Cl)cc9)CCCCC8)CC7)cc6)cc5S(=O)(=O)C(F)(F)F)CC4)C3)c(F)c2)C(=O)N1]

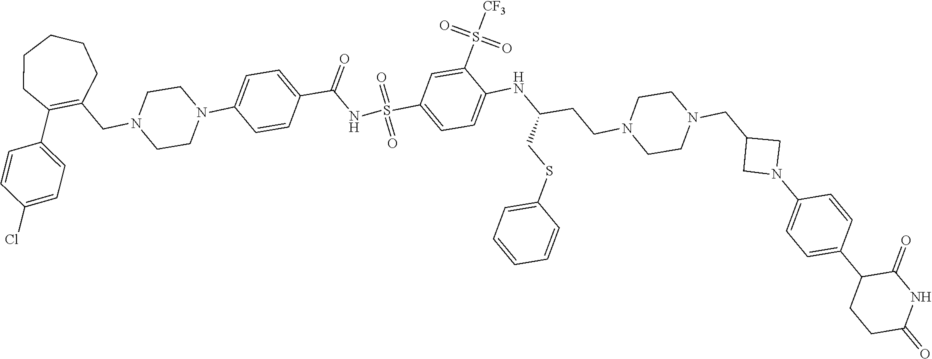 [SMILES: O=C1CCC(c2ccc(N3CC(CN4CCN(CC[C@H](CSc5ccccc5)Nc5ccc(S(=O)(=O)NC(=O)c6ccc(N7CCN(CC8=C(c9ccc(Cl)cc9)CCCCC8)CC7)cc6)cc5S(=O)(=O)C(F)(F)F)CC4)C3)cc2)C(=O)N1]

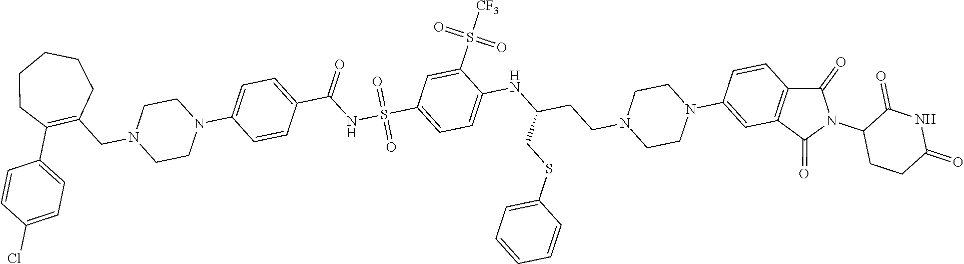 [SMILES: O=C1CCC(N2C(=O)c3ccc(N4CCN(CC[C@H](CSc5ccccc5)Nc5ccc(S(=O)(=O)NC(=O)c6ccc(N7CCN(CC8=C(c9ccc(Cl)cc9)CCCCC8)CC7)cc6)cc5S(=O)(=O)C(F)(F)F)CC4)cc3C2=O)C(=O)N1]

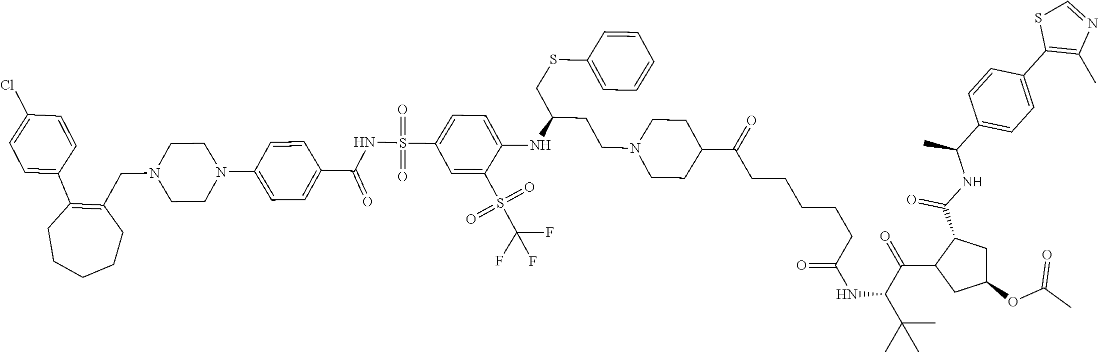 [SMILES: CC(=O)O[C@H]1CC(C(=O)[C@@H](NC(=O)CCCCCC(=O)C2CCN(CC[C@H](CSc3ccccc3)Nc3ccc(S(=O)(=O)NC(=O)c4ccc(N5CCN(CC6=C(c7ccc(Cl)cc7)CCCCC6)CC5)cc4)cc3S(=O)(=O)C(F)(F)F)CC2)C(C)(C)C)[C@H](C(=O)N[C@@H](C)c2ccc(-c3scnc3C)cc2)C1]

TABLE B-1
L group

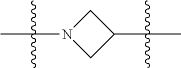 [SMILES: *C1CN(*)C1]

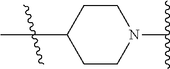 [SMILES: *C1CCN(*)CC1]

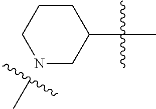

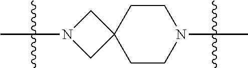 [SMILES: *N1CCC2(CC1)CN(*)C2]

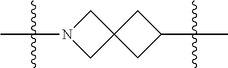 [SMILES: *C1CC2(C1)CN(*)C2]

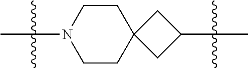 [SMILES: *C1CC2(CCN(*)CC2)C1]

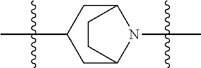 [SMILES: *C1CC2CCC(C1)N2*]

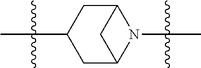 [SMILES: *C1CC2CC(C1)N2*]

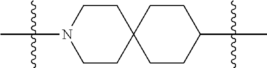 [SMILES: *C1CCC2(CC1)CCN(*)CC2]

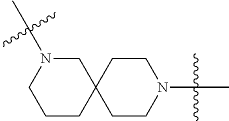 [SMILES: *N1CCC2(CCCN(*)C2)CC1]

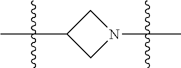 [SMILES: *C1CN(*)C1]

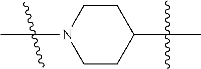 [SMILES: *C1CCN(*)CC1]

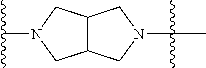 [SMILES: *N1CC2CN(*)CC2C1]

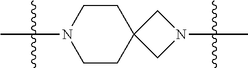 [SMILES: *N1CCC2(CC1)CN(*)C2]

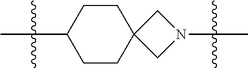 [SMILES: *C1CCC2(CC1)CN(*)C2]

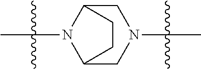 [SMILES: *N1CC2CCC(C1)N2*]

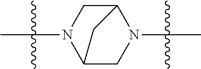 [SMILES: *N1CC2CC1CN2*]

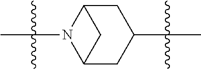 [SMILES: *C1CC2CC(C1)N2*]

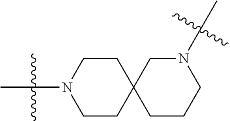 [SMILES: *N1CCC2(CCCN(*)C2)CC1]

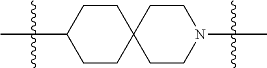 [SMILES: *C1CCC2(CC1)CCN(*)CC2]

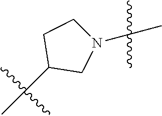 [SMILES: *C1CCN(*)C1]

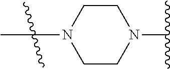 [SMILES: *N1CCN(*)CC1]

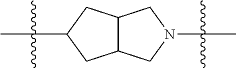 [SMILES: *C1CC2CN(*)CC2C1]

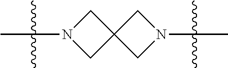 [SMILES: *N1CC2(C1)CN(*)C2]

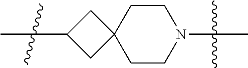 [SMILES: *C1CC2(CCN(*)CC2)C1]

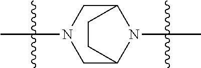 [SMILES: *N1CC2CCC(C1)N2*]

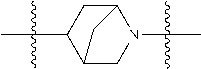 [SMILES: *C1CC2CC1CN2*]

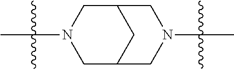 [SMILES: *N1CC2CC(C1)CN(*)C2]

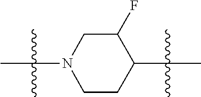 [SMILES: *C1CCN(*)CC1F]

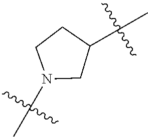 [SMILES: *C1CCN(*)C1]

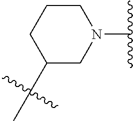 [SMILES: *C1CCCN(*)C1]

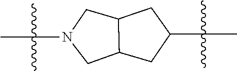 [SMILES: *C1CC2CN(*)CC2C1]

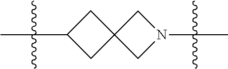 [SMILES: *C1CC2(C1)CN(*)C2]

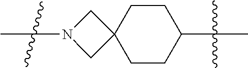 [SMILES: *C1CCC2(CC1)CN(*)C2]

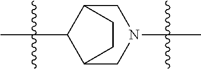 [SMILES: *C1C2CCC1CN(*)C2]

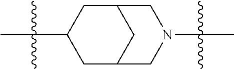 [SMILES: *C1CC2CC(C1)CN(*)C2]

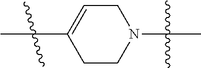 [SMILES: *C1=CCN(*)CC1]

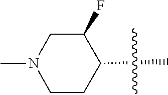

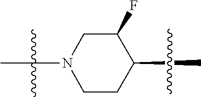

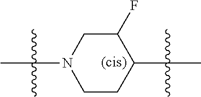 [SMILES: *C1CCN(*)CC1F]

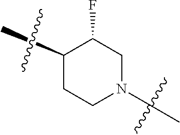

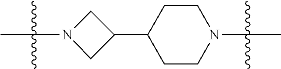 [SMILES: *N1CCC(C2CN(*)C2)CC1]

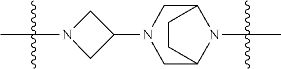 [SMILES: *N1CC(N2CC3CCC(C2)N3*)C1]

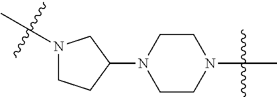 [SMILES: *N1CCN(C2CCN(*)C2)CC1]

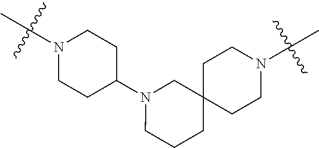 [SMILES: *N1CCC(N2CCCC3(CCN(*)CC3)C2)CC1]

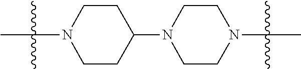 [SMILES: *N1CCC(N2CCN(*)CC2)CC1]

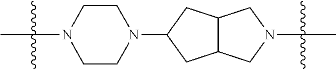 [SMILES: *N1CCN(C2CC3CN(*)CC3C2)CC1]

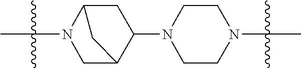 [SMILES: *N1CCN(C2CC3CC2CN3*)CC1]

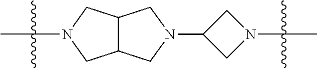 [SMILES: *N1CC2CN(C3CN(*)C3)CC2C1]

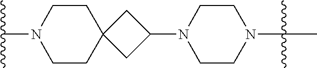 [SMILES: *N1CCN(C2CC3(CCN(*)CC3)C2)CC1]

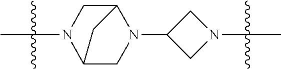 [SMILES: *N1CC(N2CC3CC2CN3*)C1]

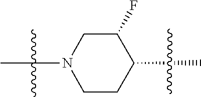

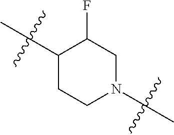 [SMILES: *C1CCN(*)CC1F]

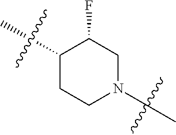

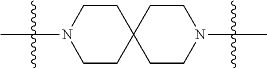 [SMILES: *N1CCC2(CC1)CCN(*)CC2]

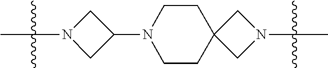 [SMILES: *N1CC(N2CCC3(CC2)CN(*)C3)C1]

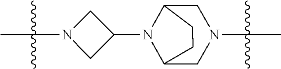 [SMILES: *N1CC2CCC(C1)N2C1CN(*)C1]

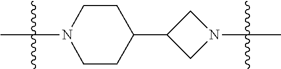 [SMILES: *N1CCC(C2CN(*)C2)CC1]

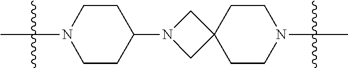 [SMILES: *N1CCC(N2CC3(CCN(*)CC3)C2)CC1]

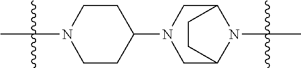 [SMILES: *N1CCC(N2CC3CCC(C2)N3*)CC1]

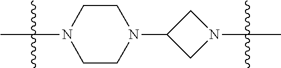 [SMILES: *N1CCN(C2CN(*)C2)CC1]

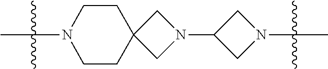 [SMILES: *N1CCC2(CC1)CN(C1CN(*)C1)C2]

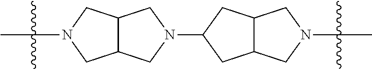 [SMILES: *N1CC2CC(N3CC4CN(*)CC4C3)CC2C1]

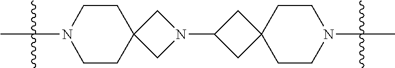 [SMILES: *N1CCC2(CC1)CC(N1CC3(CCN(*)CC3)C1)C2]

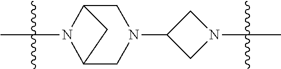 [SMILES: *N1CC(N2CC3CC(C2)N3*)C1]

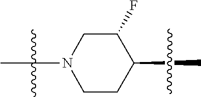

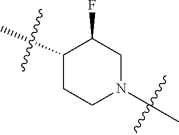

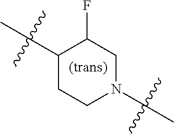 [SMILES: *C1CCN(*)CC1F]

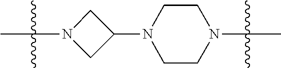 [SMILES: *N1CCN(C2CN(*)C2)CC1]

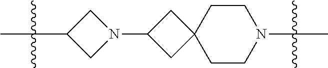 [SMILES: *C1CN(C2CC3(CCN(*)CC3)C2)C1]

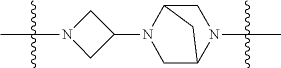 [SMILES: *N1CC(N2CC3CC2CN3*)C1]

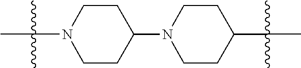 [SMILES: *C1CCN(C2CCN(*)CC2)CC1]

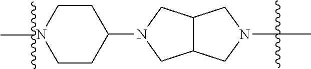

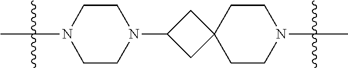 [SMILES: *N1CCN(C2CC3(CCN(*)CC3)C2)CC1]

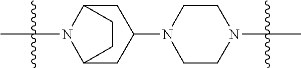 [SMILES: *N1CCN(C2CC3CCC(C2)N3*)CC1]

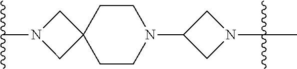 [SMILES: *N1CC(N2CCC3(CC2)CN(*)C3)C1]

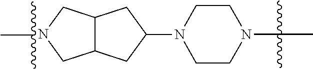

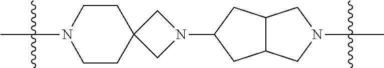 [SMILES: *N1CCC2(CC1)CN(C1CC3CN(*)CC3C1)C2]

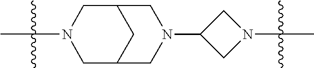 [SMILES: *N1CC2CC(C1)CN(C1CN(*)C1)C2]

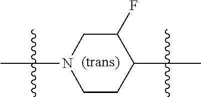 [SMILES: *C1CCN(*)CC1F]

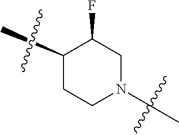

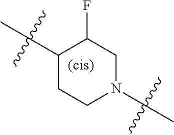 [SMILES: *C1CCN(*)CC1F]

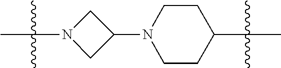 [SMILES: *C1CCN(C2CN(*)C2)CC1]

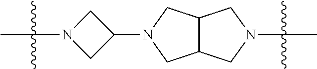 [SMILES: *N1CC2CN(C3CN(*)C3)CC2C1]

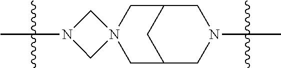

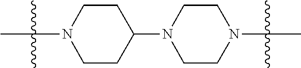 [SMILES: *N1CCC(N2CCN(*)CC2)CC1]

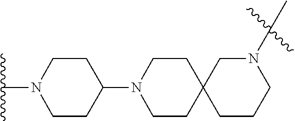 [SMILES: *N1CCC(N2CCC3(CCCN(*)C3)CC2)CC1]

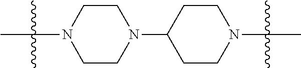 [SMILES: *N1CCC(N2CCN(*)CC2)CC1]

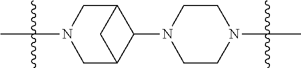 [SMILES: *N1CCN(C2C3CC2CN(*)C3)CC1]

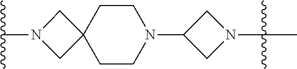 [SMILES: *N1CC(N2CCC3(CC2)CN(*)C3)C1]

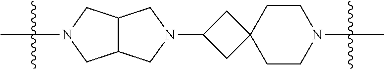 [SMILES: *N1CCC2(CC1)CC(N1CC3CN(*)CC3C1)C2]

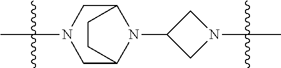 [SMILES: *N1CC2CCC(C1)N2C1CN(*)C1]

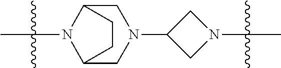 [SMILES: *N1CC(N2CC3CCC(C2)N3*)C1]

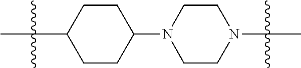 [SMILES: *C1CCC(N2CCN(*)CC2)CC1]

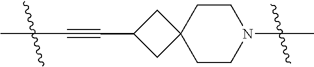 [SMILES: *C#CC1CC2(CCN(*)CC2)C1]

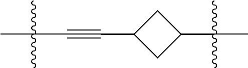 [SMILES: *C#CC1CC(*)C1]

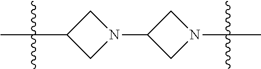 [SMILES: *C1CN(C2CN(*)C2)C1]

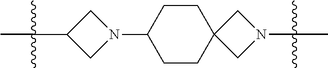 [SMILES: *C1CN(C2CCC3(CC2)CN(*)C3)C1]

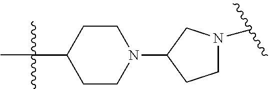 [SMILES: *C1CCN(C2CCN(*)C2)CC1]

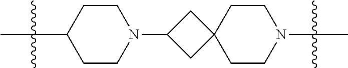 [SMILES: *C1CCN(C2CC3(CCN(*)CC3)C2)CC1]

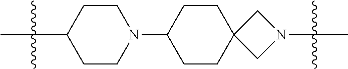 [SMILES: *C1CCN(C2CCC3(CC2)CN(*)C3)CC1]

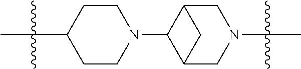 [SMILES: *C1CCN(C2C3CC2CN(*)C3)CC1]

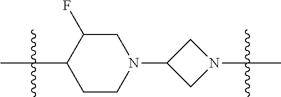 [SMILES: *C1CCN(C2CN(*)C2)CC1F]

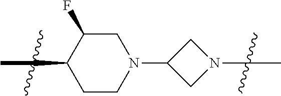 [SMILES: *[C@@H]1CCN(C2CN(*)C2)C[C@@H]1F]

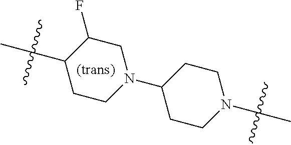 [SMILES: *C1CCN(C2CCN(*)CC2)CC1F]

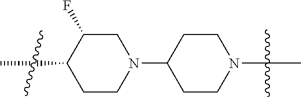 [SMILES: *[C@H]1CCN(C2CCN(*)CC2)C[C@H]1F]

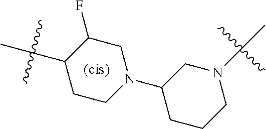 [SMILES: *C1CCN(C2CCCN(*)C2)CC1F]

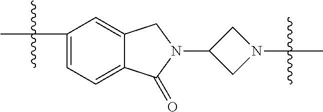 [SMILES: *c1ccc2c(c1)CN(C1CN(*)C1)C2=O]

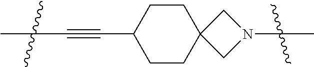 [SMILES: *C#CC1CCC2(CC1)CN(*)C2]

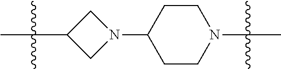 [SMILES: *C1CN(C2CCN(*)CC2)C1]

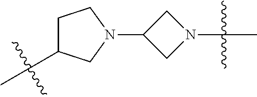 [SMILES: *C1CCN(C2CN(*)C2)C1]

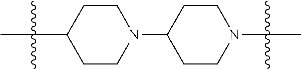 [SMILES: *C1CCN(C2CCN(*)CC2)CC1]

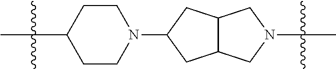 [SMILES: *C1CCN(C2CC3CN(*)CC3C2)CC1]

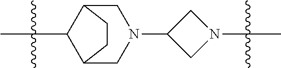 [SMILES: *C1C2CCC1CN(C1CN(*)C1)C2]

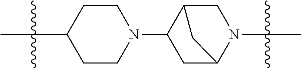 [SMILES: *C1CCN(C2CC3CC2CN3*)CC1]

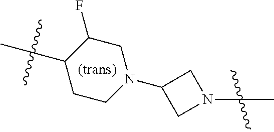 [SMILES: *C1CCN(C2CN(*)C2)CC1F]

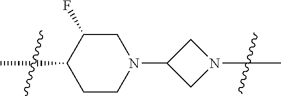 [SMILES: *[C@H]1CCN(C2CN(*)C2)C[C@H]1F]

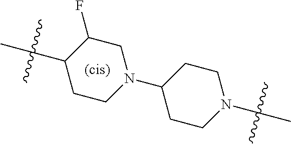 [SMILES: *C1CCN(C2CCN(*)CC2)CC1F]

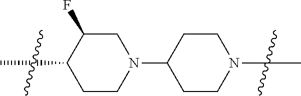 [SMILES: *[C@H]1CCN(C2CCN(*)CC2)C[C@@H]1F]

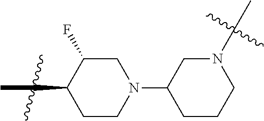 [SMILES: *[C@@H]1CCN(C2CCCN(*)C2)C[C@H]1F]

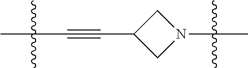 [SMILES: *C#CC1CN(*)C1]

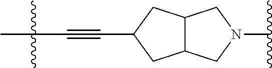 [SMILES: *C#CC1CC2CN(*)CC2C1]

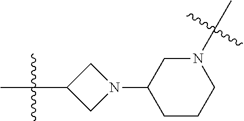 [SMILES: *C1CN(C2CCCN(*)C2)C1]

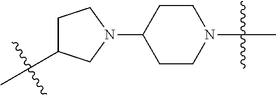 [SMILES: *C1CCN(C2CCN(*)CC2)C1]

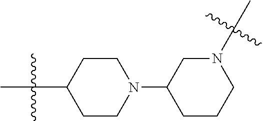 [SMILES: *C1CCN(C2CCCN(*)C2)CC1]

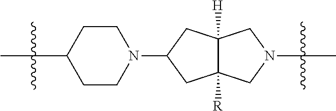

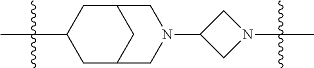 [SMILES: *C1CC2CC(C1)CN(C1CN(*)C1)C2]

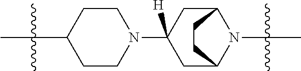 [SMILES: *C1CCN([C@@H]2C[C@H]3CC[C@@H](C2)N3*)CC1]

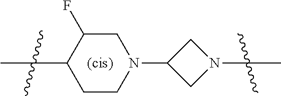 [SMILES: *C1CCN(C2CN(*)C2)CC1F]

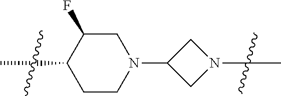 [SMILES: *[C@H]1CCN(C2CN(*)C2)C[C@@H]1F]

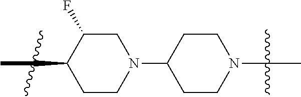 [SMILES: *[C@@H]1CCN(C2CCN(*)CC2)C[C@H]1F]

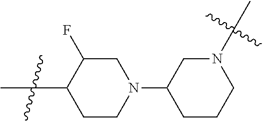 [SMILES: *C1CCN(C2CCCN(*)C2)CC1F]

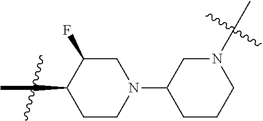 [SMILES: *[C@@H]1CCN(C2CCCN(*)C2)C[C@@H]1F]

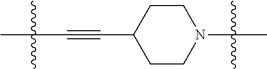 [SMILES: *C#CC1CCN(*)CC1]

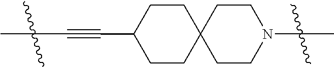 [SMILES: *C#CC1CCC2(CC1)CCN(*)CC2]

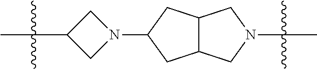 [SMILES: *C1CN(C2CC3CN(*)CC3C2)C1]

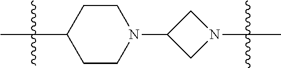 [SMILES: *C1CCN(C2CN(*)C2)CC1]

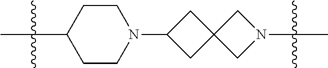 [SMILES: *C1CCN(C2CC3(C2)CN(*)C3)CC1]

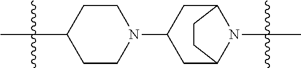 [SMILES: *C1CCN(C2CC3CCC(C2)N3*)CC1]

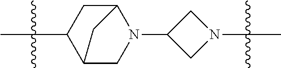 [SMILES: *C1CC2CC1CN2C1CN(*)C1]

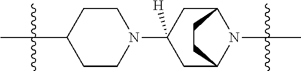 [SMILES: *C1CCN([C@H]2C[C@H]3CC[C@@H](C2)N3*)CC1]

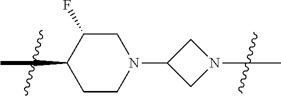 [SMILES: *[C@@H]1CCN(C2CN(*)C2)C[C@H]1F]

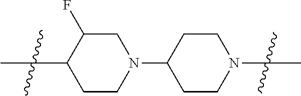 [SMILES: *C1CCN(C2CCN(*)CC2)CC1F]

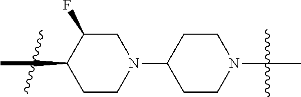 [SMILES: *[C@@H]1CCN(C2CCN(*)CC2)C[C@@H]1F]

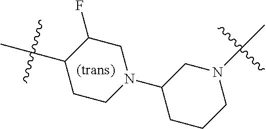 [SMILES: *C1CCN(C2CCCN(*)C2)CC1F]

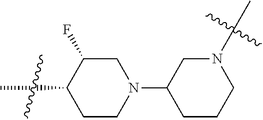 [SMILES: *[C@H]1CCN(C2CCCN(*)C2)C[C@H]1F]

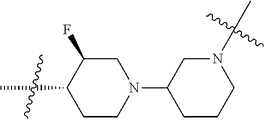 [SMILES: *[C@H]1CCN(C2CCCN(*)C2)C[C@@H]1F]

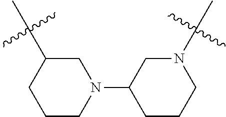 [SMILES: *C1CCCN(C2CCCN(*)C2)C1]

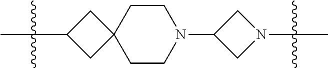 [SMILES: *C1CC2(CCN(C3CN(*)C3)CC2)C1]

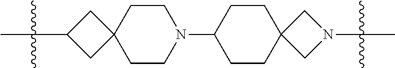 [SMILES: *C1CC2(CCN(C3CCC4(CC3)CN(*)C4)CC2)C1]

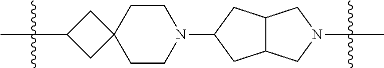 [SMILES: *C1CC2(CCN(C3CC4CN(*)CC4C3)CC2)C1]

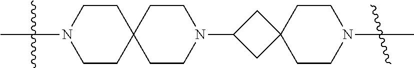 [SMILES: *N1CCC2(CC1)CCN(C1CC3(CCN(*)CC3)C1)CC2]

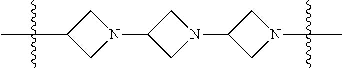 [SMILES: *C1CN(C2CN(C3CN(*)C3)C2)C1]

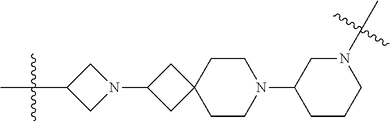 [SMILES: *C1CN(C2CC3(CCN(C4CCCN(*)C4)CC3)C2)C1]

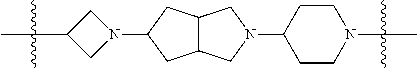 [SMILES: *C1CN(C2CC3CN(C4CCN(*)CC4)CC3C2)C1]

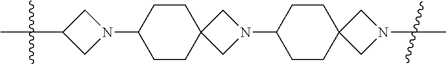 [SMILES: *C1CN(C2CCC3(CC2)CN(C2CCC4(CC2)CN(*)C4)C3)C1]

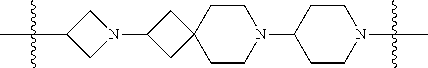 [SMILES: *C1CN(C2CC3(CCN(C4CCN(*)CC4)CC3)C2)C1]

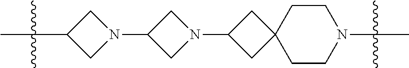 [SMILES: *C1CN(C2CN(C3CC4(CCN(*)CC4)C3)C2)C1]

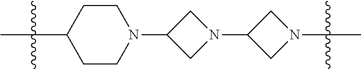 [SMILES: *C1CCN(C2CN(C3CN(*)C3)C2)CC1]

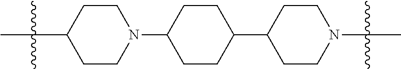 [SMILES: *C1CCN(C2CCC(C3CCN(*)CC3)CC2)CC1]

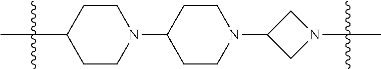 [SMILES: *C1CCN(C2CCN(C3CN(*)C3)CC2)CC1]

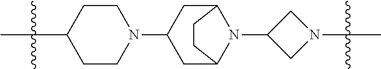 [SMILES: *C1CCN(C2CC3CCC(C2)N3C2CN(*)C2)CC1]

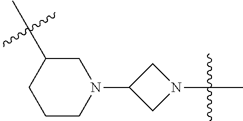 [SMILES: *C1CCCN(C2CN(*)C2)C1]

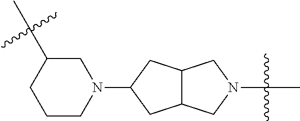 [SMILES: *C1CCCN(C2CC3CN(*)CC3C2)C1]

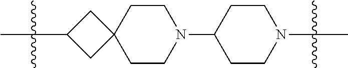 [SMILES: *C1CC2(CCN(C3CCN(*)CC3)CC2)C1]

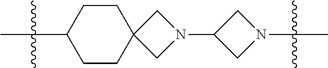 [SMILES: *C1CCC2(CC1)CN(C1CN(*)C1)C2]

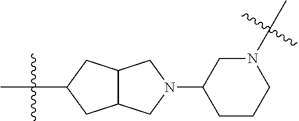 [SMILES: *C1CC2CN(C3CCCN(*)C3)CC2C1]

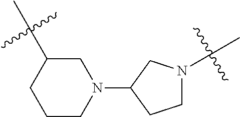 [SMILES: *C1CCCN(C2CCN(*)C2)C1]

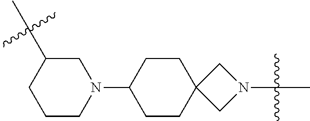 [SMILES: *C1CCCN(C2CCC3(CC2)CN(*)C3)C1]

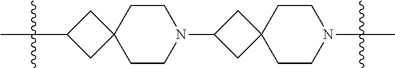 [SMILES: *C1CC2(CCN(C3CC4(CCN(*)CC4)C3)CC2)C1]

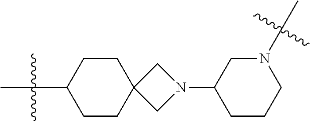 [SMILES: *C1CCC2(CC1)CN(C1CCCN(*)C1)C2]

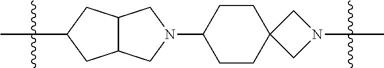 [SMILES: *C1CC2CN(C3CCC4(CC3)CN(*)C4)CC2C1]

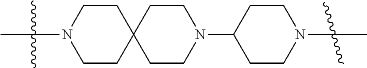 [SMILES: *N1CCC(N2CCC3(CCN(*)CC3)CC2)CC1]

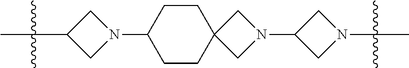 [SMILES: *C1CN(C2CCC3(CC2)CN(C2CN(*)C2)C3)C1]

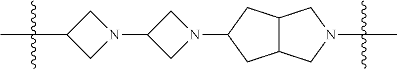 [SMILES: *C1CN(C2CN(C3CC4CN(*)CC4C3)C2)C1]

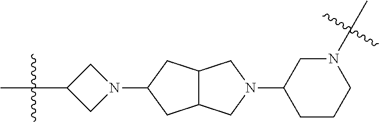 [SMILES: *C1CN(C2CC3CN(C4CCCN(*)C4)CC3C2)C1]

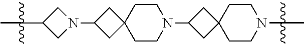 [SMILES: *C1CN(C2CC3(CCN(C4CC5(CCN(*)CC5)C4)CC3)C2)C1]

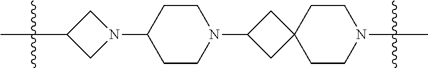 [SMILES: *C1CN(C2CCN(C3CC4(CCN(*)CC4)C3)CC2)C1]

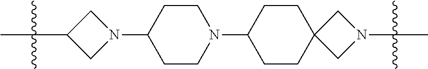 [SMILES: *C1CN(C2CCN(C3CCC4(CC3)CN(*)C4)CC2)C1]

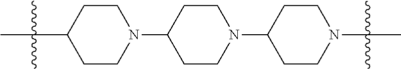 [SMILES: *C1CCN(C2CCN(C3CCN(*)CC3)CC2)CC1]

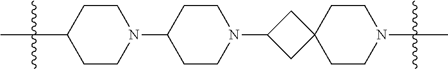 [SMILES: *C1CCN(C2CCN(C3CC4(CCN(*)CC4)C3)CC2)CC1]

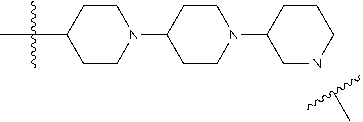

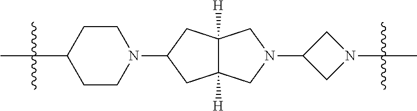 [SMILES: *C1CCN(C2C[C@@H]3CN(C4CN(*)C4)C[C@@H]3C2)CC1]

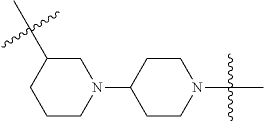 [SMILES: *C1CCCN(C2CCN(*)CC2)C1]

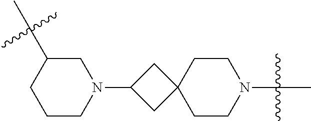 [SMILES: *C1CCCN(C2CC3(CCN(*)CC3)C2)C1]

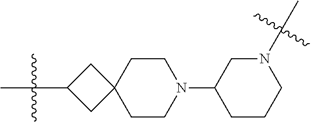 [SMILES: *C1CC2(CCN(C3CCCN(*)C3)CC2)C1]

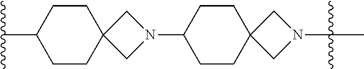 [SMILES: *C1CCC2(CC1)CN(C1CCC3(CC1)CN(*)C3)C2]

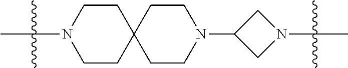 [SMILES: *N1CCC2(CC1)CCN(C1CN(*)C1)CC2]

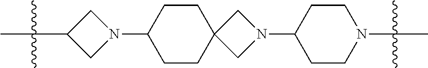 [SMILES: *C1CN(C2CCC3(CC2)CN(C2CCN(*)CC2)C3)C1]

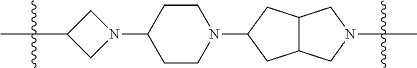 [SMILES: *C1CN(C2CCN(C3CC4CN(*)CC4C3)CC2)C1]

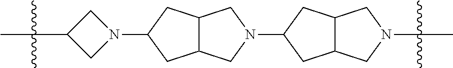 [SMILES: *C1CN(C2CC3CN(C4CC5CN(*)CC5C4)CC3C2)C1]

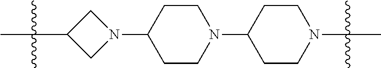 [SMILES: *C1CN(C2CCN(C3CCN(*)CC3)CC2)C1]

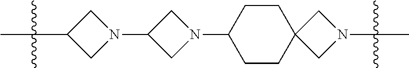 [SMILES: *C1CN(C2CN(C3CCC4(CC3)CN(*)C4)C2)C1]

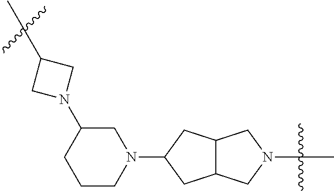 [SMILES: *C1CN(C2CCCN(C3CC4CN(*)CC4C3)C2)C1]

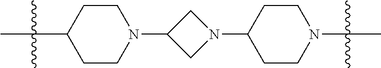 [SMILES: *C1CCN(C2CN(C3CCN(*)CC3)C2)CC1]

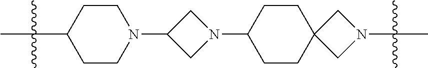 [SMILES: *C1CCN(C2CN(C3CCC4(CC3)CN(*)C4)C2)CC1]

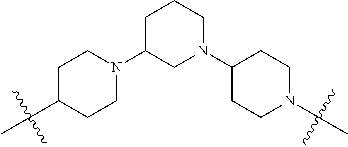 [SMILES: *C1CCN(C2CCCN(C3CCN(*)CC3)C2)CC1]

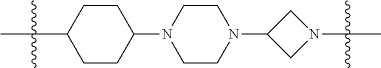 [SMILES: *C1CCC(N2CCN(C3CN(*)C3)CC2)CC1]

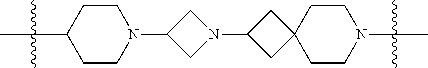 [SMILES: *C1CCN(C2CN(C3CC4(CCN(*)CC4)C3)C2)CC1]

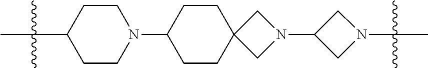 [SMILES: *C1CCN(C2CCC3(CC2)CN(C2CN(*)C2)C3)CC1]

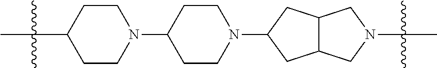 [SMILES: *C1CCN(C2CCN(C3CC4CN(*)CC4C3)CC2)CC1]

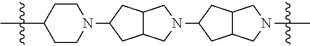 [SMILES: *C1CCN(C2CC3CN(C4CC5CN(*)CC5C4)CC3C2)CC1]

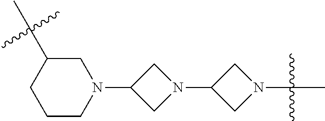 [SMILES: *C1CCCN(C2CN(C3CN(*)C3)C2)C1]

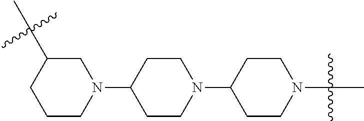 [SMILES: *C1CCCN(C2CCN(C3CCN(*)CC3)CC2)C1]

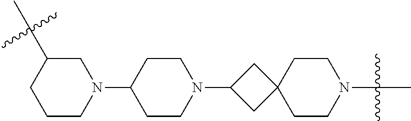 [SMILES: *C1CCCN(C2CCN(C3CC4(CCN(*)CC4)C3)CC2)C1]

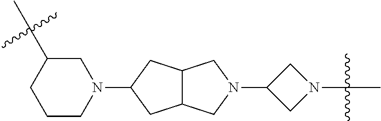 [SMILES: *C1CCCN(C2CC3CN(C4CN(*)C4)CC3C2)C1]

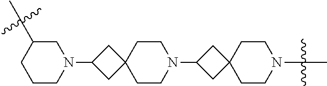 [SMILES: *C1CCCN(C2CC3(CCN(C4CC5(CCN(*)CC5)C4)CC3)C2)C1]

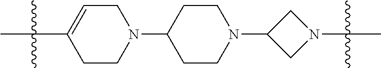 [SMILES: *C1=CCN(C2CCN(C3CN(*)C3)CC2)CC1]

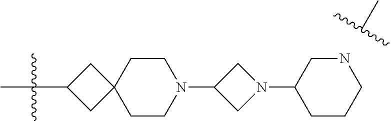

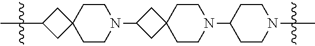 [SMILES: *C1CC2(CCN(C3CC4(CCN(C5CCN(*)CC5)CC4)C3)CC2)C1]

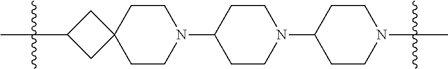 [SMILES: *C1CC2(CCN(C3CCN(C4CCN(*)CC4)CC3)CC2)C1]

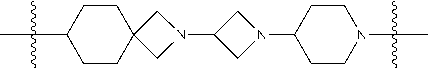 [SMILES: *C1CCC2(CC1)CN(C1CN(C3CCN(*)CC3)C1)C2]

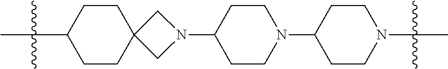 [SMILES: *C1CCC2(CC1)CN(C1CCN(C3CCN(*)CC3)CC1)C2]

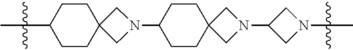 [SMILES: *C1CCC2(CC1)CN(C1CCC3(CC1)CN(C1CN(*)C1)C3)C2]

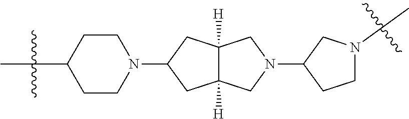 [SMILES: *C1CCN(C2C[C@@H]3CN(C4CCN(*)C4)C[C@@H]3C2)CC1]

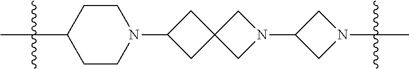 [SMILES: *C1CCN(C2CC3(C2)CN(C2CN(*)C2)C3)CC1]

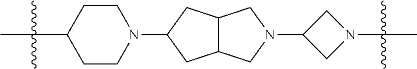 [SMILES: *C1CCN(C2CC3CN(C4CN(*)C4)CC3C2)CC1]

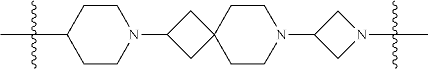 [SMILES: *C1CCN(C2CC3(CCN(C4CN(*)C4)CC3)C2)CC1]

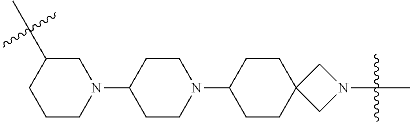 [SMILES: *C1CCCN(C2CCN(C3CCC4(CC3)CN(*)C4)CC2)C1]

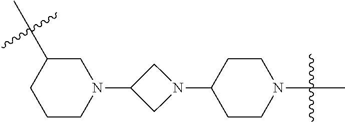 [SMILES: *C1CCCN(C2CN(C3CCN(*)CC3)C2)C1]

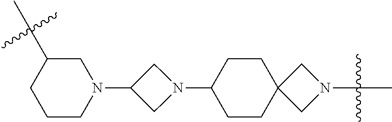 [SMILES: *C1CCCN(C2CN(C3CCC4(CC3)CN(*)C4)C2)C1]

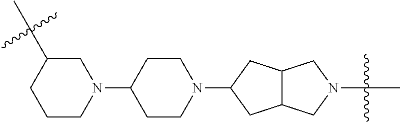 [SMILES: *C1CCCN(C2CCN(C3CC4CN(*)CC4C3)CC2)C1]

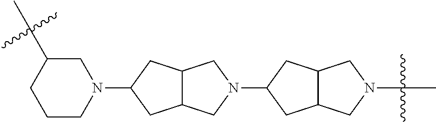 [SMILES: *C1CCCN(C2CC3CN(C4CC5CN(*)CC5C4)CC3C2)C1]

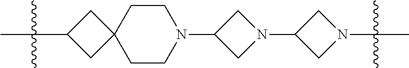 [SMILES: *C1CC2(CCN(C3CN(C4CN(*)C4)C3)CC2)C1]

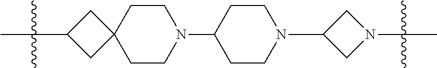 [SMILES: *C1CC2(CCN(C3CCN(C4CN(*)C4)CC3)CC2)C1]

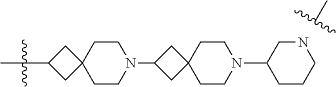

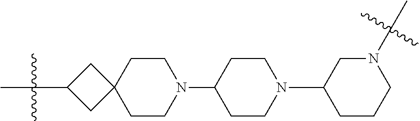 [SMILES: *C1CC2(CCN(C3CCN(C4CCCN(*)C4)CC3)CC2)C1]

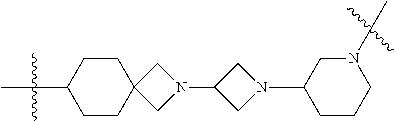 [SMILES: *C1CCC2(CC1)CN(C1CN(C3CCCN(*)C3)C1)C2]

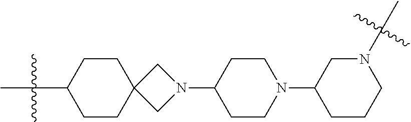 [SMILES: *C1CCC2(CC1)CN(C1CCN(C3CCCN(*)C3)CC1)C2]

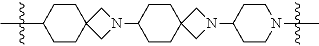 [SMILES: *C1CCC2(CC1)CN(C1CCC3(CC1)CN(C1CCN(*)CC1)C3)C2]

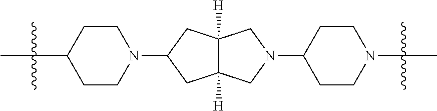 [SMILES: *C1CCN(C2C[C@@H]3CN(C4CCN(*)CC4)C[C@@H]3C2)CC1]

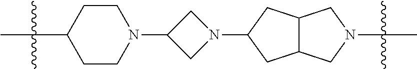 [SMILES: *C1CCN(C2CN(C3CC4CN(*)CC4C3)C2)CC1]

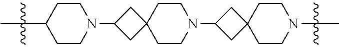 [SMILES: *C1CCN(C2CC3(CCN(C4CC5(CCN(*)CC5)C4)CC3)C2)CC1]

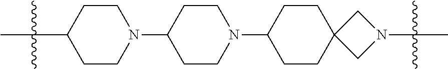 [SMILES: *C1CCN(C2CCN(C3CCC4(CC3)CN(*)C4)CC2)CC1]

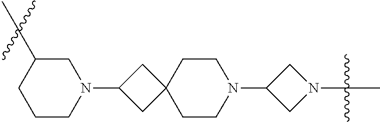 [SMILES: *C1CCCN(C2CC3(CCN(C4CN(*)C4)CC3)C2)C1]

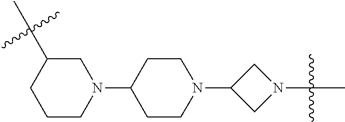 [SMILES: *C1CCCN(C2CCN(C3CN(*)C3)CC2)C1]

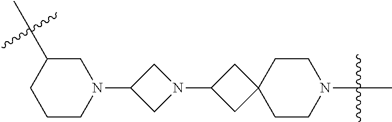 [SMILES: *C1CCCN(C2CN(C3CC4(CCN(*)CC4)C3)C2)C1]

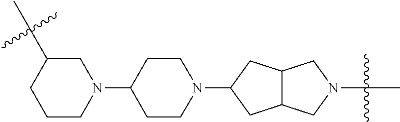 [SMILES: *C1CCCN(C2CCN(C3CC4CN(*)CC4C3)CC2)C1]

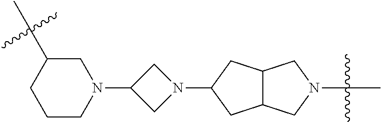 [SMILES: *C1CCCN(C2CN(C3CC4CN(*)CC4C3)C2)C1]

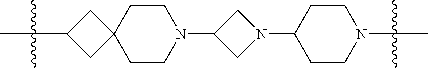 [SMILES: *C1CC2(CCN(C3CN(C4CCN(*)CC4)C3)CC2)C1]

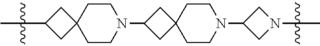 [SMILES: *C1CC2(CCN(C3CC4(CCN(C5CN(*)C5)CC4)C3)CC2)C1]

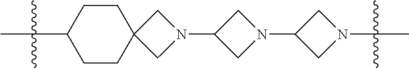 [SMILES: *C1CCC2(CC1)CN(C1CN(C3CN(*)C3)C1)C2]

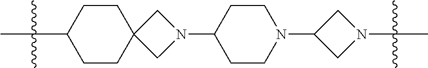 [SMILES: *C1CCC2(CC1)CN(C1CCN(C3CN(*)C3)CC1)C2]

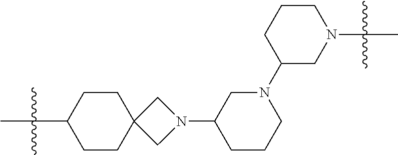 [SMILES: *C1CCC2(CC1)CN(C1CCCN(C3CCCN(*)C3)C1)C2]

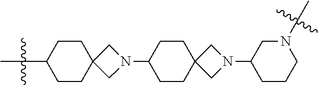 [SMILES: *C1CCC2(CC1)CN(C1CCC3(CC1)CN(C1CCCN(*)C1)C3)C2]

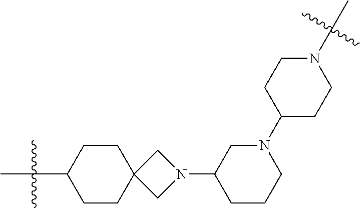 [SMILES: *C1CCC2(CC1)CN(C1CCCN(C3CCN(*)CC3)C1)C2]

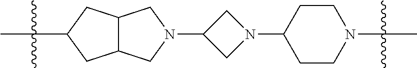 [SMILES: *C1CC2CN(C3CN(C4CCN(*)CC4)C3)CC2C1]

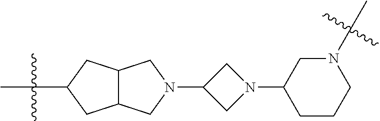 [SMILES: *C1CC2CN(C3CN(C4CCCN(*)C4)C3)CC2C1]

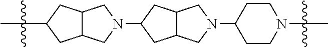 [SMILES: *C1CC2CN(C3CC4CN(C5CCN(*)CC5)CC4C3)CC2C1]

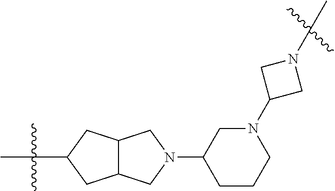 [SMILES: *C1CC2CN(C3CCCN(C4CN(*)C4)C3)CC2C1]

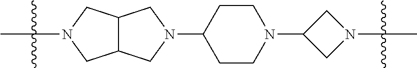 [SMILES: *N1CC2CN(C3CCN(C4CN(*)C4)CC3)CC2C1]

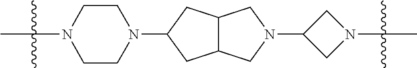 [SMILES: *N1CCN(C2CC3CN(C4CN(*)C4)CC3C2)CC1]

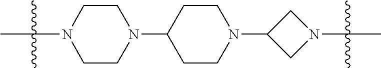 [SMILES: *N1CCN(C2CCN(C3CN(*)C3)CC2)CC1]

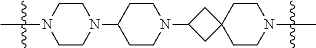 [SMILES: *N1CCN(C2CCN(C3CC4(CCN(*)CC4)C3)CC2)CC1]

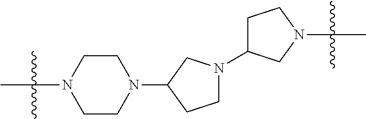 [SMILES: *N1CCN(C2CCN(C3CCN(*)C3)C2)CC1]

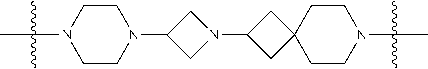 [SMILES: *N1CCN(C2CN(C3CC4(CCN(*)CC4)C3)C2)CC1]

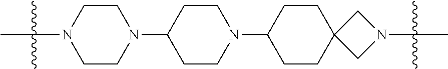 [SMILES: *N1CCN(C2CCN(C3CCC4(CC3)CN(*)C4)CC2)CC1]

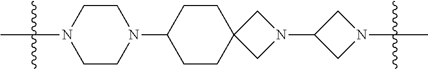 [SMILES: *N1CCN(C2CCC3(CC2)CN(C2CN(*)C2)C3)CC1]

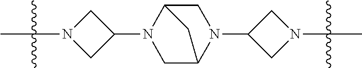 [SMILES: *N1CC(N2CC3CC2CN3C2CN(*)C2)C1]

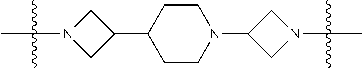 [SMILES: *N1CC(C2CCN(C3CN(*)C3)CC2)C1]

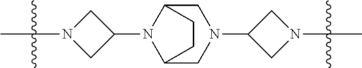 [SMILES: *N1CC(N2CC3CCC(C2)N3C2CN(*)C2)C1]

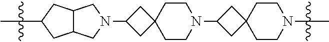 [SMILES: *C1CC2CN(C3CC4(CCN(C5CC6(CCN(*)CC6)C5)CC4)C3)CC2C1]

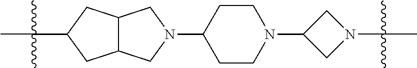 [SMILES: *C1CC2CN(C3CCN(C4CN(*)C4)CC3)CC2C1]

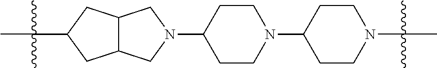 [SMILES: *C1CC2CN(C3CCN(C4CCN(*)CC4)CC3)CC2C1]

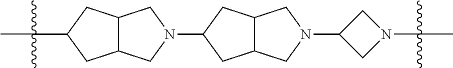 [SMILES: *C1CC2CN(C3CC4CN(C5CN(*)C5)CC4C3)CC2C1]

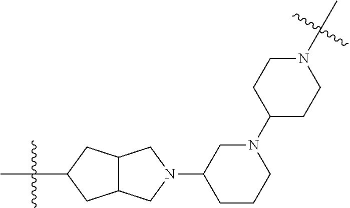 [SMILES: *C1CC2CN(C3CCCN(C4CCN(*)CC4)C3)CC2C1]

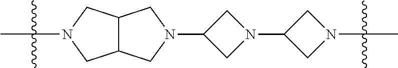 [SMILES: *N1CC2CN(C3CN(C4CN(*)C4)C3)CC2C1]

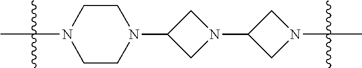 [SMILES: *N1CCN(C2CN(C3CN(*)C3)C2)CC1]

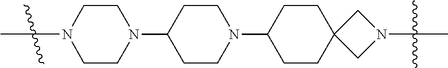 [SMILES: *N1CCN(C2CCN(C3CCC4(CC3)CN(*)C4)CC2)CC1]

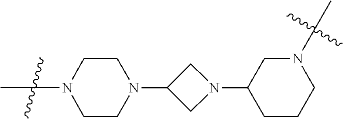 [SMILES: *N1CCN(C2CN(C3CCCN(*)C3)C2)CC1]

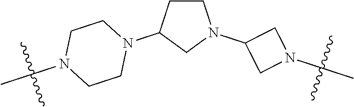 [SMILES: *N1CCN(C2CCN(C3CN(*)C3)C2)CC1]

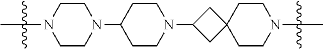 [SMILES: *N1CCN(C2CCN(C3CC4(CCN(*)CC4)C3)CC2)CC1]

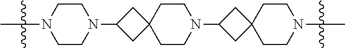 [SMILES: *N1CCN(C2CC3(CCN(C4CC5(CCN(*)CC5)C4)CC3)C2)CC1]

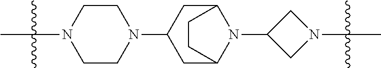 [SMILES: *N1CCN(C2CC3CCC(C2)N3C2CN(*)C2)CC1]

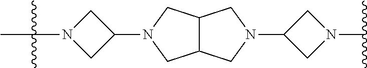 [SMILES: *N1CC(N2CC3CN(C4CN(*)C4)CC3C2)C1]

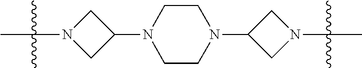 [SMILES: *N1CC(N2CCN(C3CN(*)C3)CC2)C1]

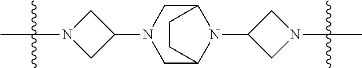 [SMILES: *N1CC(N2CC3CCC(C2)N3C2CN(*)C2)C1]

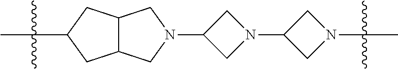 [SMILES: *C1CC2CN(C3CN(C4CN(*)C4)C3)CC2C1]

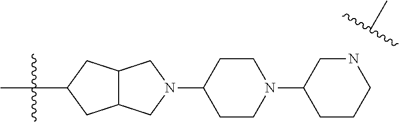

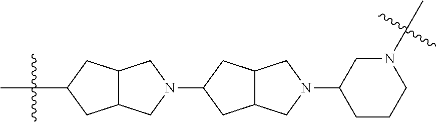 [SMILES: *C1CC2CN(C3CC4CN(C5CCCN(*)C5)CC4C3)CC2C1]

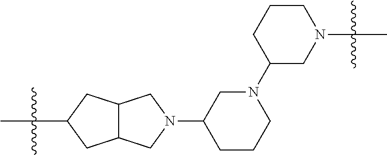 [SMILES: *C1CC2CN(C3CCCN(C4CCCN(*)C4)C3)CC2C1]

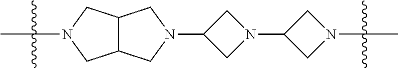 [SMILES: *N1CC2CN(C3CN(C4CN(*)C4)C3)CC2C1]

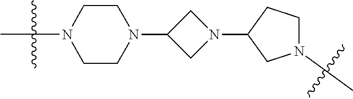 [SMILES: *N1CCN(C2CN(C3CCN(*)C3)C2)CC1]

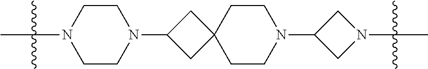 [SMILES: *N1CCN(C2CC3(CCN(C4CN(*)C4)CC3)C2)CC1]

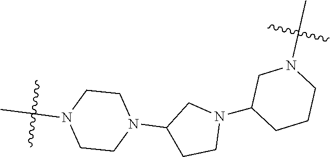 [SMILES: *N1CCN(C2CCN(C3CCCN(*)C3)C2)CC1]

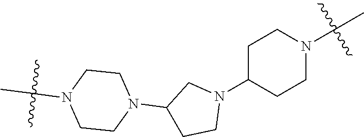 [SMILES: *N1CCC(N2CCC(N3CCN(*)CC3)C2)CC1]

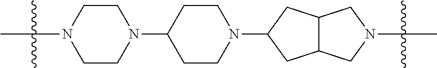 [SMILES: *N1CCN(C2CCN(C3CC4CN(*)CC4C3)CC2)CC1]

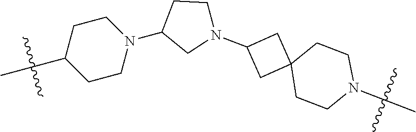 [SMILES: *C1CCN(C2CCN(C3CC4(CCN(*)CC4)C3)C2)CC1]

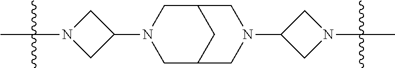 [SMILES: *N1CC(N2CC3CC(C2)CN(C2CN(*)C2)C3)C1]

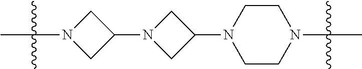 [SMILES: *N1CCN(C2CN(C3CN(*)C3)C2)CC1]

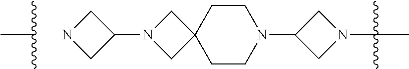

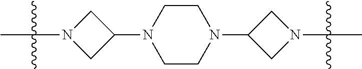 [SMILES: *N1CC(N2CCN(C3CN(*)C3)CC2)C1]

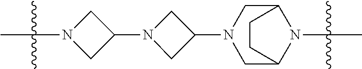 [SMILES: *N1CC(N2CC(N3CC4CCC(C3)N4*)C2)C1]

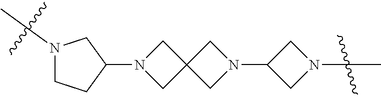 [SMILES: *N1CCC(N2CC3(C2)CN(C2CN(*)C2)C3)C1]

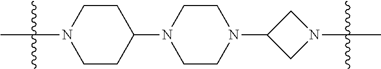 [SMILES: *N1CCC(N2CCN(C3CN(*)C3)CC2)CC1]

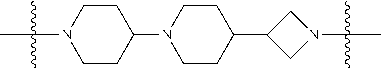 [SMILES: *N1CCC(N2CCC(C3CN(*)C3)CC2)CC1]

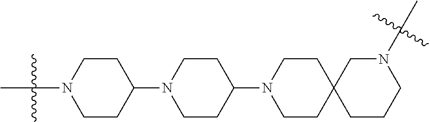 [SMILES: *N1CCC(N2CCC(N3CCC4(CCCN(*)C4)CC3)CC2)CC1]

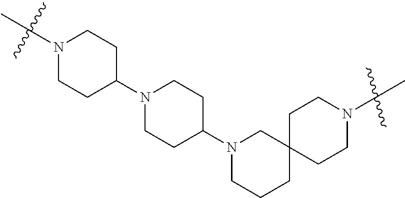 [SMILES: *N1CCC(N2CCC(N3CCCC4(CCN(*)CC4)C3)CC2)CC1]

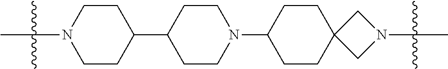 [SMILES: *N1CCC(C2CCN(C3CCC4(CC3)CN(*)C4)CC2)CC1]

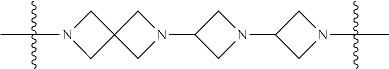 [SMILES: *N1CC(N2CC(N3CC4(CN(*)C4)C3)C2)C1]

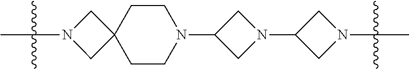 [SMILES: *N1CC(N2CC(N3CCC4(CC3)CN(*)C4)C2)C1]

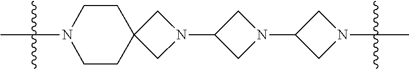 [SMILES: *N1CCC2(CC1)CN(C1CN(C3CN(*)C3)C1)C2]

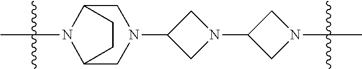 [SMILES: *N1CC(N2CC(N3CC4CCC(C3)N4*)C2)C1]

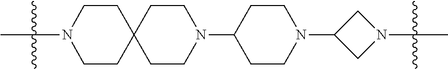 [SMILES: *N1CCC2(CC1)CCN(C1CCN(C3CN(*)C3)CC1)CC2]

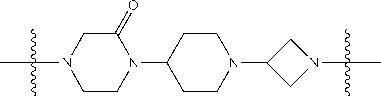 [SMILES: *N1CCN(C2CCN(C3CN(*)C3)CC2)C(=O)C1]

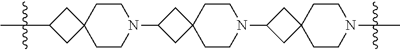 [SMILES: *C1CC2(CCN(C3CC4(CCN(C5CC6(CCN(*)CC6)C5)CC4)C3)CC2)C1]

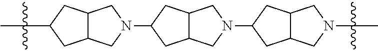 [SMILES: *C1CC2CN(C3CC4CN(C5CC6CN(*)CC6C5)CC4C3)CC2C1]

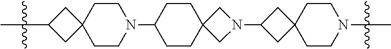 [SMILES: *C1CC2(CCN(C3CCC4(CC3)CN(C3CC5(CCN(*)CC5)C3)C4)CC2)C1]

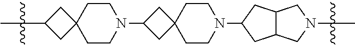 [SMILES: *C1CC2(CCN(C3CC4(CCN(C5CC6CN(*)CC6C5)CC4)C3)CC2)C1]

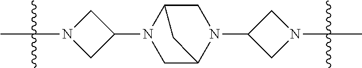 [SMILES: *N1CC(N2CC3CC2CN3C2CN(*)C2)C1]

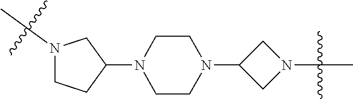 [SMILES: *N1CCC(N2CCN(C3CN(*)C3)CC2)C1]

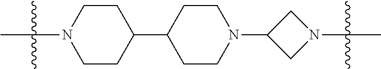 [SMILES: *N1CCC(C2CCN(C3CN(*)C3)CC2)CC1]

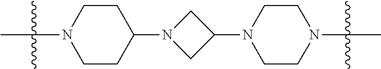 [SMILES: *N1CCC(N2CC(N3CCN(*)CC3)C2)CC1]

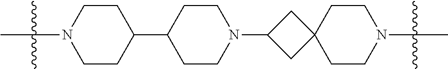 [SMILES: *N1CCC(C2CCN(C3CC4(CCN(*)CC4)C3)CC2)CC1]

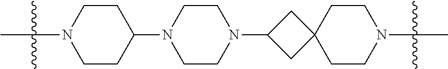 [SMILES: *N1CCC(N2CCN(C3CC4(CCN(*)CC4)C3)CC2)CC1]

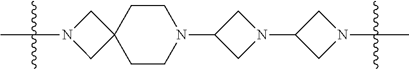 [SMILES: *N1CC(N2CC(N3CCC4(CC3)CN(*)C4)C2)C1]

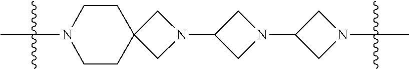 [SMILES: *N1CCC2(CC1)CN(C1CN(C3CN(*)C3)C1)C2]

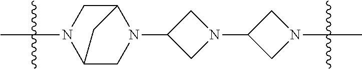 [SMILES: *N1CC(N2CC(N3CC4CC3CN4*)C2)C1]

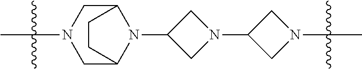 [SMILES: *N1CC(N2CC(N3C4CCC3CN(*)C4)C2)C1]

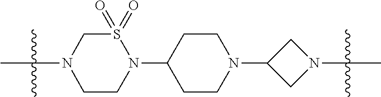 [SMILES: *N1CC(N2CCC(N3CCN(*)CS3(=O)=O)CC2)C1]

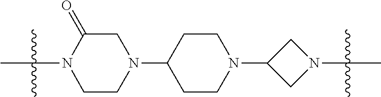 [SMILES: *N1CC(N2CCC(N3CCN(*)C(=O)C3)CC2)C1]

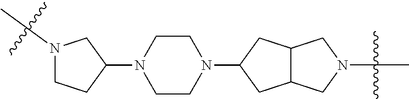 [SMILES: *N1CC2CC(N3CCN(C4CCN(*)C4)CC3)CC2C1]

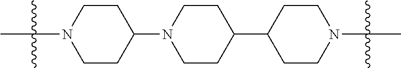 [SMILES: *N1CCC(C2CCN(C3CCN(*)CC3)CC2)CC1]

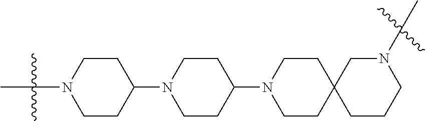 [SMILES: *N1CCC(N2CCC(N3CCC4(CCCN(*)C4)CC3)CC2)CC1]

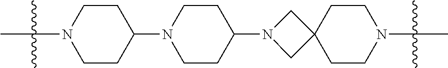 [SMILES: *N1CCC(N2CCC(N3CC4(CCN(*)CC4)C3)CC2)CC1]

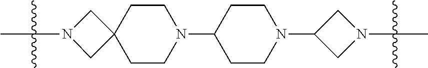 [SMILES: *N1CC(N2CCC(N3CCC4(CC3)CN(*)C4)CC2)C1]

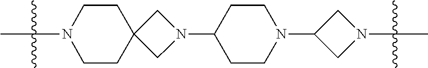 [SMILES: *N1CCC2(CC1)CN(C1CCN(C3CN(*)C3)CC1)C2]

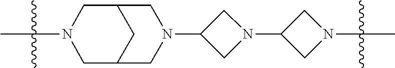 [SMILES: *N1CC2CC(C1)CN(C1CN(C3CN(*)C3)C1)C2]

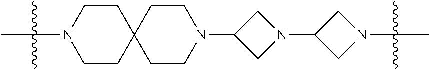 [SMILES: *N1CCC2(CC1)CCN(C1CN(C3CN(*)C3)C1)CC2]

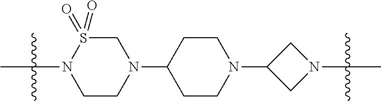 [SMILES: *N1CC(N2CCC(N3CCN(*)S(=O)(=O)C3)CC2)C1]

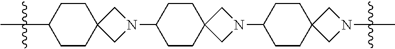 [SMILES: *C1CCC2(CC1)CN(C1CCC3(CC1)CN(C1CCC4(CC1)CN(*)C4)C3)C2]

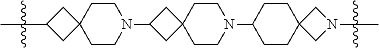 [SMILES: *C1CC2(CCN(C3CC4(CCN(C5CCC6(CC5)CN(*)C6)CC4)C3)CC2)C1]

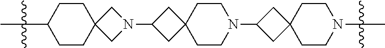 [SMILES: *C1CCC2(CC1)CN(C1CC3(CCN(C4CC5(CCN(*)CC5)C4)CC3)C1)C2]

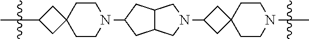 [SMILES: *C1CC2(CCN(C3CC4CN(C5CC6(CCN(*)CC6)C5)CC4C3)CC2)C1]

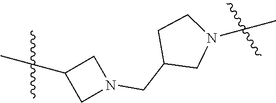 [SMILES: *C1CN(CC2CCN(*)C2)C1]

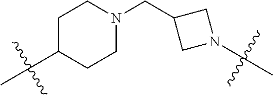 [SMILES: *C1CCN(CC2CN(*)C2)CC1]

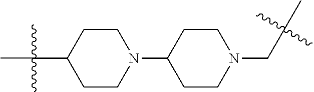 [SMILES: *CN1CCC(N2CCC(*)CC2)CC1]

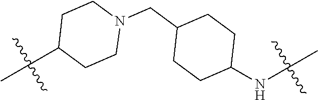 [SMILES: *NC1CCC(CN2CCC(*)CC2)CC1]

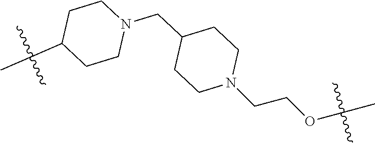 [SMILES: *OCCN1CCC(CN2CCC(*)CC2)CC1]

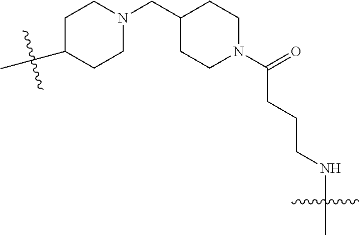 [SMILES: *NCCCC(=O)N1CCC(CN2CCC(*)CC2)CC1]

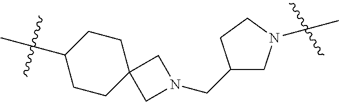 [SMILES: *C1CCC2(CC1)CN(CC1CCN(*)C1)C2]

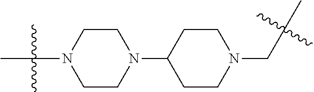 [SMILES: *CN1CCC(N2CCN(*)CC2)CC1]

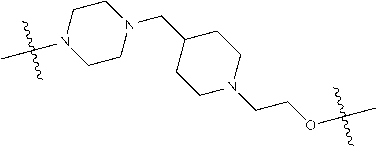 [SMILES: *OCCN1CCC(CN2CCN(*)CC2)CC1]

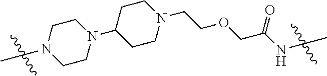 [SMILES: *NC(=O)COCCN1CCC(N2CCN(*)CC2)CC1]

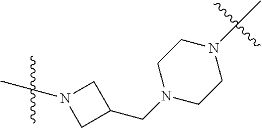 [SMILES: *N1CCN(CC2CN(*)C2)CC1]

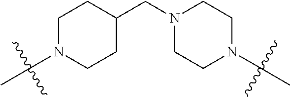 [SMILES: *N1CCC(CN2CCN(*)CC2)CC1]

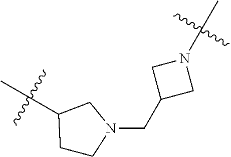 [SMILES: *C1CCN(CC2CN(*)C2)C1]

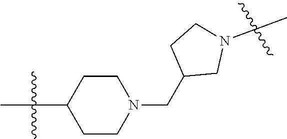 [SMILES: *C1CCN(CC2CCN(*)C2)CC1]

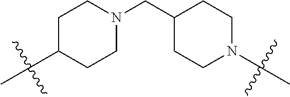 [SMILES: *C1CCN(CC2CCN(*)CC2)CC1]

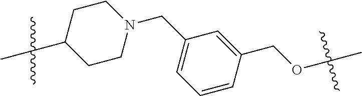 [SMILES: *OCc1cccc(CN2CCC(*)CC2)c1]

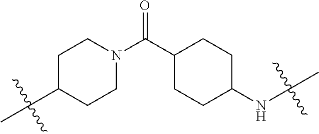 [SMILES: *NC1CCC(C(=O)N2CCC(*)CC2)CC1]

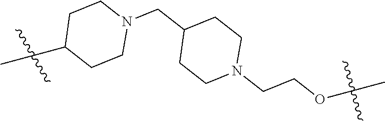 [SMILES: *OCCN1CCC(CN2CCC(*)CC2)CC1]

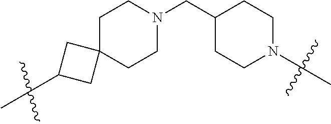 [SMILES: *C1CC2(CCN(CC3CCN(*)CC3)CC2)C1]

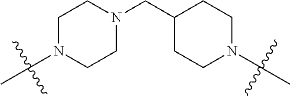 [SMILES: *N1CCC(CN2CCN(*)CC2)CC1]

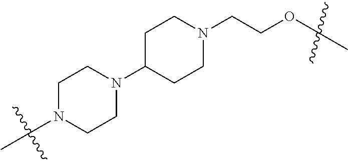 [SMILES: *OCCN1CCC(N2CCN(*)CC2)CC1]

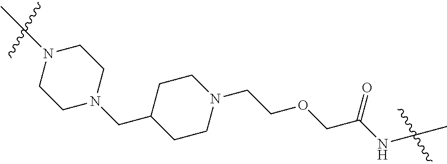 [SMILES: *NC(=O)COCCN1CCC(CN2CCN(*)CC2)CC1]

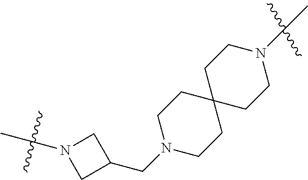 [SMILES: *N1CCC2(CC1)CCN(CC1CN(*)C1)CC2]

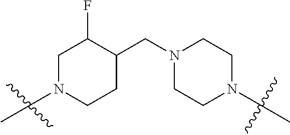 [SMILES: *N1CCN(CC2CCN(*)CC2F)CC1]

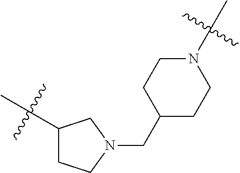 [SMILES: *C1CCN(CC2CCN(*)CC2)C1]

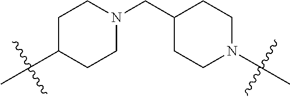 [SMILES: *C1CCN(CC2CCN(*)CC2)CC1]

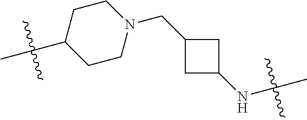 [SMILES: *NC1CC(CN2CCC(*)CC2)C1]

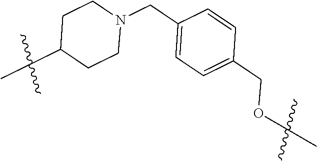 [SMILES: *OCc1ccc(CN2CCC(*)CC2)cc1]

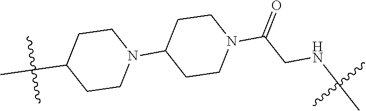 [SMILES: *NCC(=O)N1CCC(N2CCC(*)CC2)CC1]

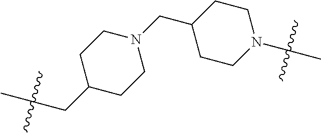 [SMILES: *CC1CCN(CC2CCN(*)CC2)CC1]

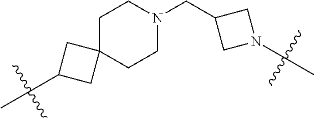 [SMILES: *C1CC2(CCN(CC3CN(*)C3)CC2)C1]

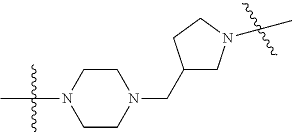 [SMILES: *N1CCN(CC2CCN(*)C2)CC1]

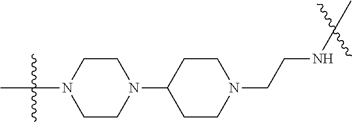 [SMILES: *NCCN1CCC(N2CCN(*)CC2)CC1]

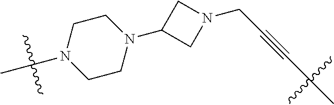 [SMILES: *C#CCN1CC(N2CCN(*)CC2)C1]

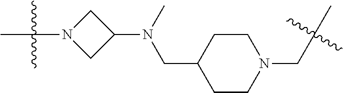 [SMILES: *CN1CCC(CN(C)C2CN(*)C2)CC1]

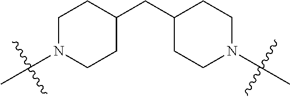 [SMILES: *N1CCC(CC2CCN(*)CC2)CC1]

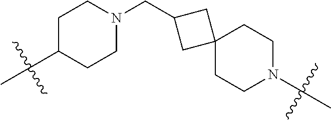 [SMILES: *C1CCN(CC2CC3(CCN(*)CC3)C2)CC1]

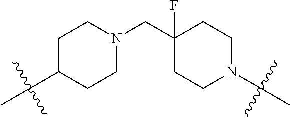 [SMILES: *C1CCN(CC2(F)CCN(*)CC2)CC1]

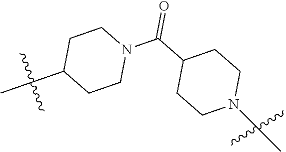 [SMILES: *C1CCN(C(=O)C2CCN(*)CC2)CC1]

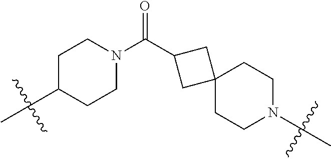 [SMILES: *C1CCN(C(=O)C2CC3(CCN(*)CC3)C2)CC1]

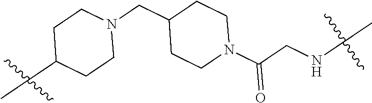 [SMILES: *NCC(=O)N1CCC(CN2CCC(*)CC2)CC1]

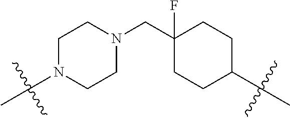 [SMILES: *C1CCC(F)(CN2CCN(*)CC2)CC1]

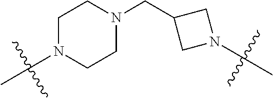 [SMILES: *N1CCN(CC2CN(*)C2)CC1]

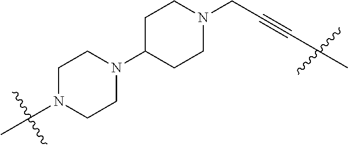 [SMILES: *C#CCN1CCC(N2CCN(*)CC2)CC1]

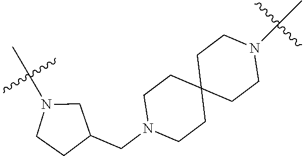 [SMILES: *N1CCC2(CC1)CCN(CC1CCN(*)C1)CC2]

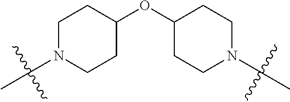 [SMILES: *N1CCC(OC2CCN(*)CC2)CC1]

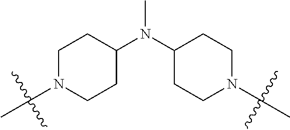 [SMILES: *N1CCC(N(C)C2CCN(*)CC2)CC1]

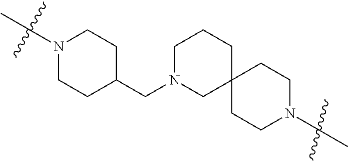 [SMILES: *N1CCC(CN2CCCC3(CCN(*)CC3)C2)CC1]

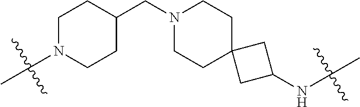 [SMILES: *NC1CC2(CCN(CC3CCN(*)CC3)CC2)C1]

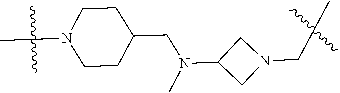 [SMILES: *CN1CC(N(C)CC2CCN(*)CC2)C1]

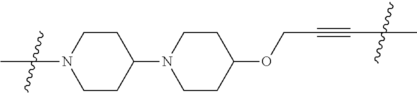 [SMILES: *C#CCOC1CCN(C2CCN(*)CC2)CC1]

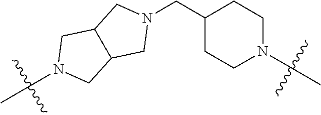 [SMILES: *N1CCC(CN2CC3CN(*)CC3C2)CC1]

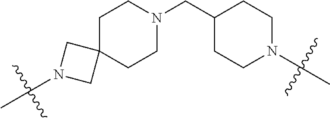 [SMILES: *N1CCC(CN2CCC3(CC2)CN(*)C3)CC1]

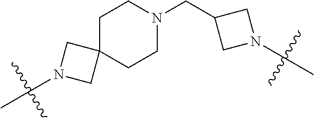 [SMILES: *N1CC(CN2CCC3(CC2)CN(*)C3)C1]

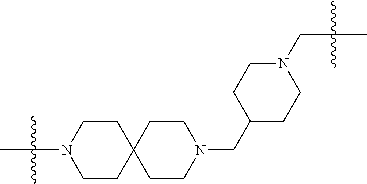 [SMILES: *CN1CCC(CN2CCC3(CCN(*)CC3)CC2)CC1]

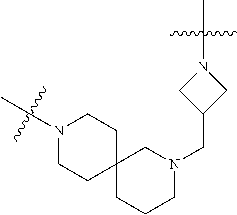 [SMILES: *N1CCC2(CCCN(CC3CN(*)C3)C2)CC1]

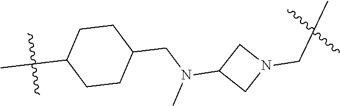 [SMILES: *CN1CC(N(C)CC2CCC(*)CC2)C1]

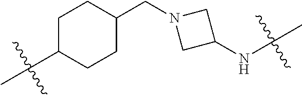 [SMILES: *NC1CN(CC2CCC(*)CC2)C1]

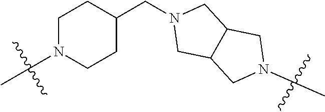 [SMILES: *N1CCC(CN2CC3CN(*)CC3C2)CC1]

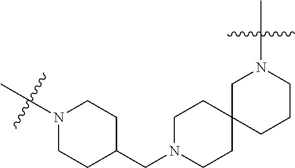 [SMILES: *N1CCC(CN2CCC3(CCCN(*)C3)CC2)CC1]

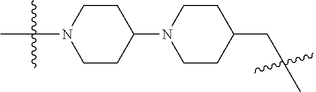 [SMILES: *CC1CCN(C2CCN(*)CC2)CC1]

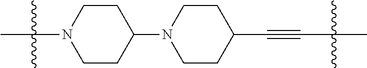 [SMILES: *C#CC1CCN(C2CCN(*)CC2)CC1]

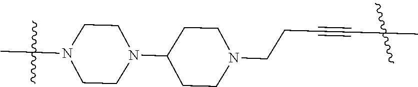 [SMILES: *C#CCCN1CCC(N2CCN(*)CC2)CC1]

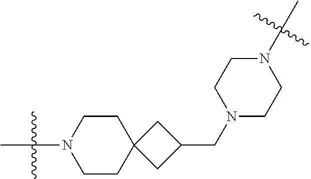 [SMILES: *N1CCN(CC2CC3(CCN(*)CC3)C2)CC1]

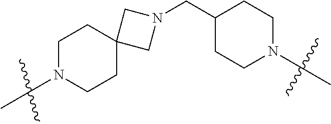 [SMILES: *N1CCC(CN2CC3(CCN(*)CC3)C2)CC1]

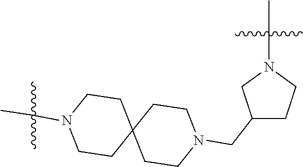 [SMILES: *N1CCC2(CC1)CCN(CC1CCN(*)C1)CC2]

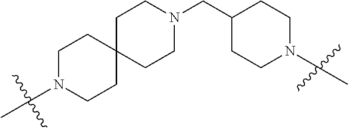 [SMILES: *N1CCC(CN2CCC3(CCN(*)CC3)CC2)CC1]

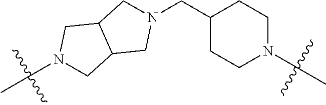 [SMILES: *N1CCC(CN2CC3CN(*)CC3C2)CC1]

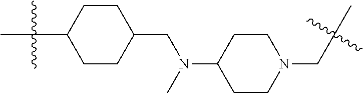 [SMILES: *CN1CCC(N(C)CC2CCC(*)CC2)CC1]

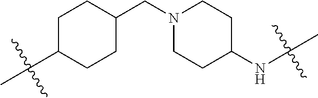 [SMILES: *NC1CCN(CC2CCC(*)CC2)CC1]

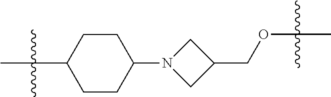 [SMILES: *OCC1CN(C2CCC(*)CC2)C1]

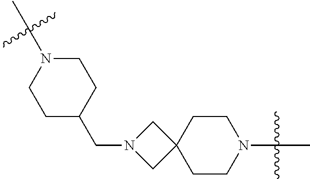 [SMILES: *N1CCC(CN2CC3(CCN(*)CC3)C2)CC1]

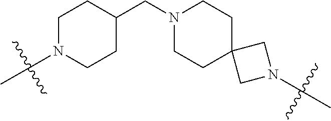 [SMILES: *N1CCC(CN2CCC3(CC2)CN(*)C3)CC1]

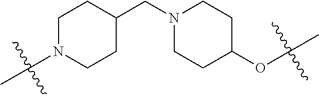 [SMILES: *OC1CCN(CC2CCN(*)CC2)CC1]

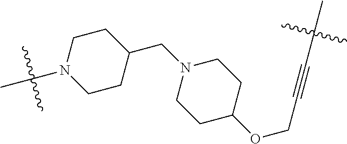 [SMILES: *C#CCOC1CCN(CC2CCN(*)CC2)CC1]

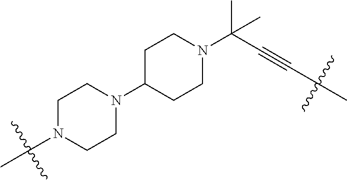 [SMILES: *C#CC(C)(C)N1CCC(N2CCN(*)CC2)CC1]

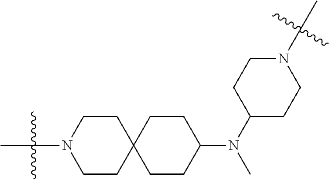 [SMILES: *N1CCC(N(C)C2CCC3(CC2)CCN(*)CC3)CC1]

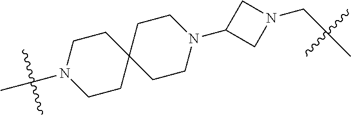 [SMILES: *CN1CC(N2CCC3(CCN(*)CC3)CC2)C1]

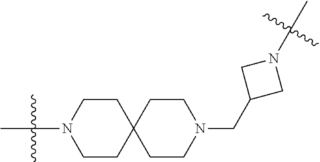 [SMILES: *N1CCC2(CC1)CCN(CC1CN(*)C1)CC2]

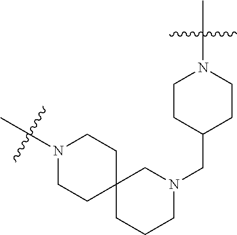 [SMILES: *N1CCC(CN2CCCC3(CCN(*)CC3)C2)CC1]

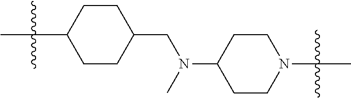 [SMILES: *C1CCC(CN(C)C2CCN(*)CC2)CC1]

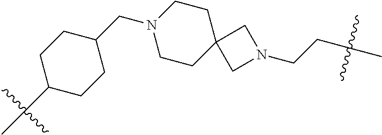 [SMILES: *CCN1CC2(CCN(CC3CCC(*)CC3)CC2)C1]

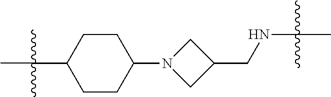 [SMILES: *NCC1CN(C2CCC(*)CC2)C1]

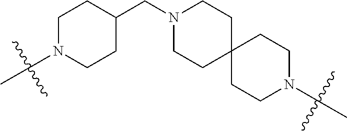 [SMILES: *N1CCC(CN2CCC3(CCN(*)CC3)CC2)CC1]

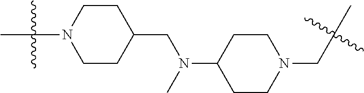 [SMILES: *CN1CCC(N(C)CC2CCN(*)CC2)CC1]

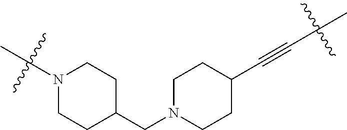 [SMILES: *C#CC1CCN(CC2CCN(*)CC2)CC1]

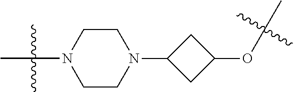 [SMILES: *OC1CC(N2CCN(*)CC2)C1]

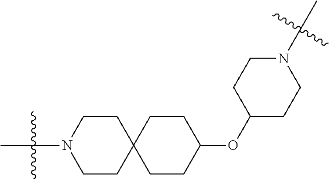 [SMILES: *N1CCC(OC2CCC3(CC2)CCN(*)CC3)CC1]

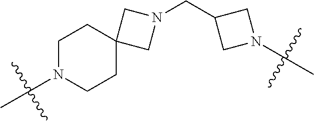 [SMILES: *N1CCC2(CC1)CN(CC1CN(*)C1)C2]

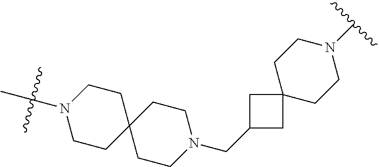 [SMILES: *N1CCC2(CC1)CCN(CC1CC3(CCN(*)CC3)C1)CC2]

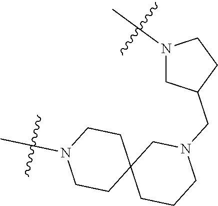 [SMILES: *N1CCC2(CCCN(CC3CCN(*)C3)C2)CC1]

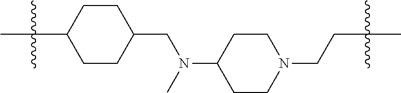 [SMILES: *CCN1CCC(N(C)CC2CCC(*)CC2)CC1]

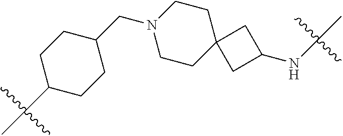 [SMILES: *NC1CC2(CCN(CC3CCC(*)CC3)CC2)C1]

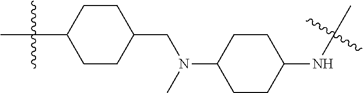 [SMILES: *NC1CCC(N(C)CC2CCC(*)CC2)CC1]

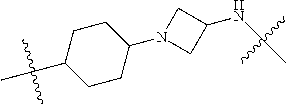 [SMILES: *NC1CN(C2CCC(*)CC2)C1]

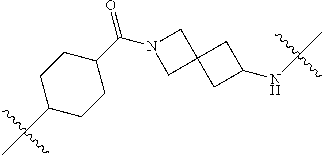 [SMILES: *NC1CC2(C1)CN(C(=O)C1CCC(*)CC1)C2]

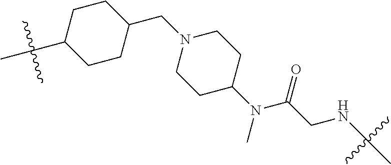 [SMILES: *NCC(=O)N(C)C1CCN(CC2CCC(*)CC2)CC1]

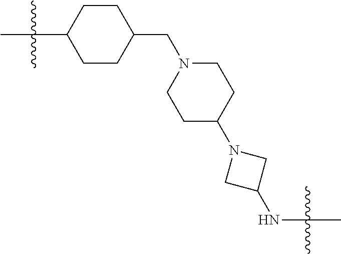 [SMILES: *NC1CN(C2CCN(CC3CCC(*)CC3)CC2)C1]

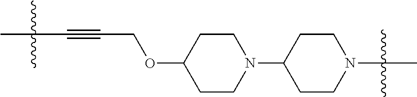 [SMILES: *C#CCOC1CCN(C2CCN(*)CC2)CC1]

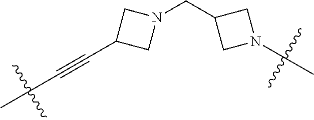 [SMILES: *C#CC1CN(CC2CN(*)C2)C1]

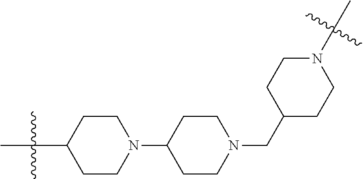 [SMILES: *C1CCN(C2CCN(CC3CCN(*)CC3)CC2)CC1]

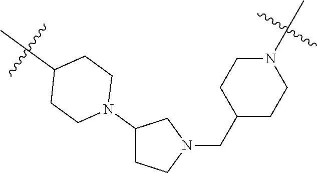 [SMILES: *C1CCN(C2CCN(CC3CCN(*)CC3)C2)CC1]

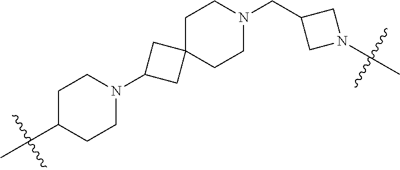 [SMILES: *C1CCN(C2CC3(CCN(CC4CN(*)C4)CC3)C2)CC1]

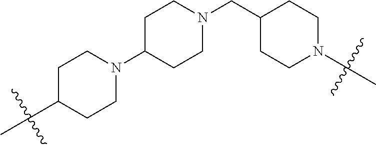 [SMILES: *C1CCN(C2CCN(CC3CCN(*)CC3)CC2)CC1]

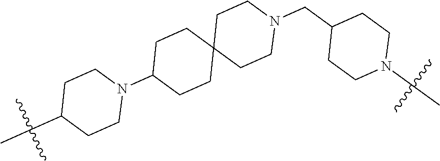 [SMILES: *C1CCN(C2CCC3(CC2)CCN(CC2CCN(*)CC2)CC3)CC1]

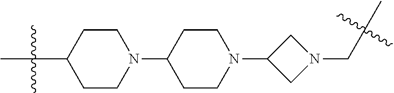 [SMILES: *CN1CC(N2CCC(N3CCC(*)CC3)CC2)C1]

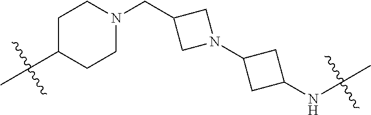 [SMILES: *NC1CC(N2CC(CN3CCC(*)CC3)C2)C1]

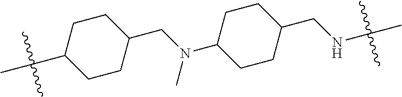 [SMILES: *NCC1CCC(N(C)CC2CCC(*)CC2)CC1]

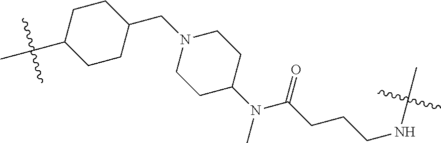 [SMILES: *NCCCC(=O)N(C)C1CCN(CC2CCC(*)CC2)CC1]

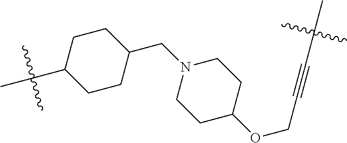 [SMILES: *C#CCOC1CCN(CC2CCC(*)CC2)CC1]

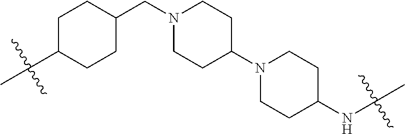 [SMILES: *NC1CCN(C2CCN(CC3CCC(*)CC3)CC2)CC1]

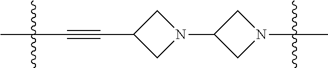 [SMILES: *C#CC1CN(C2CN(*)C2)C1]

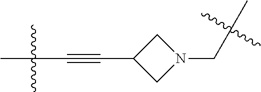 [SMILES: *C#CC1CN(C*)C1]

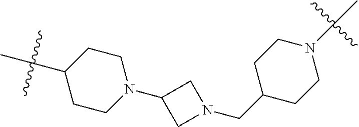 [SMILES: *C1CCN(C2CN(CC3CCN(*)CC3)C2)CC1]

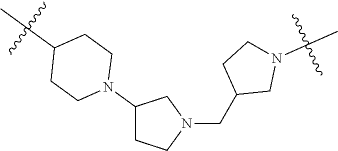 [SMILES: *C1CCN(C2CCN(CC3CCN(*)C3)C2)CC1]

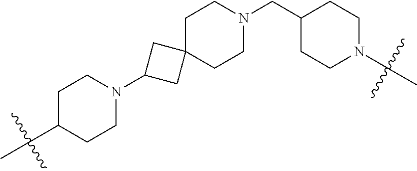 [SMILES: *C1CCN(C2CC3(CCN(CC4CCN(*)CC4)CC3)C2)CC1]

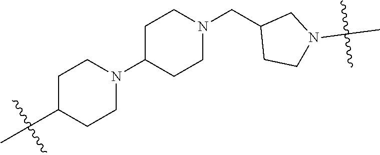 [SMILES: *C1CCN(C2CCN(CC3CCN(*)C3)CC2)CC1]

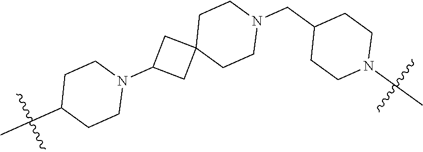 [SMILES: *C1CCN(C2CC3(CCN(CC4CCN(*)CC4)CC3)C2)CC1]

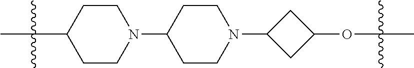 [SMILES: *OC1CC(N2CCC(N3CCC(*)CC3)CC2)C1]

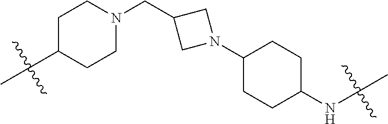 [SMILES: *NC1CCC(N2CC(CN3CCC(*)CC3)C2)CC1]

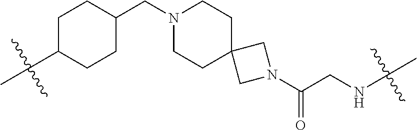 [SMILES: *NCC(=O)N1CC2(CCN(CC3CCC(*)CC3)CC2)C1]

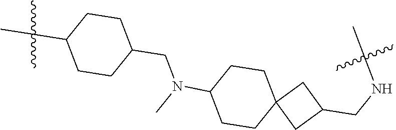 [SMILES: *NCC1CC2(CCC(N(C)CC3CCC(*)CC3)CC2)C1]

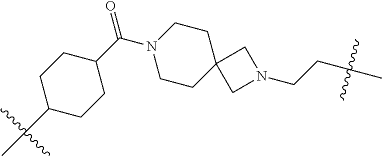 [SMILES: *CCN1CC2(CCN(C(=O)C3CCC(*)CC3)CC2)C1]

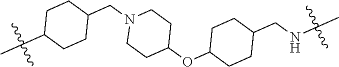 [SMILES: *NCC1CCC(OC2CCN(CC3CCC(*)CC3)CC2)CC1]

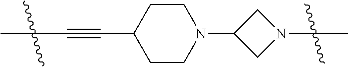 [SMILES: *C#CC1CCN(C2CN(*)C2)CC1]

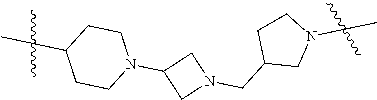 [SMILES: *C1CCN(C2CN(CC3CCN(*)C3)C2)CC1]

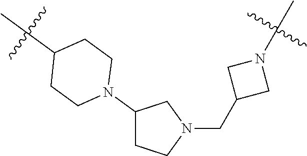 [SMILES: *C1CCN(C2CCN(CC3CN(*)C3)C2)CC1]

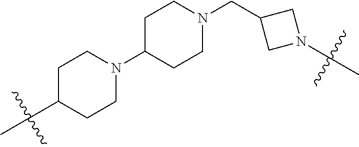 [SMILES: *C1CCN(C2CCN(CC3CN(*)C3)CC2)CC1]

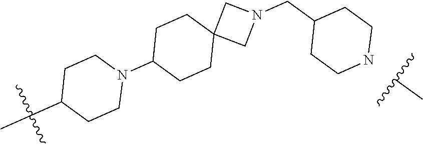

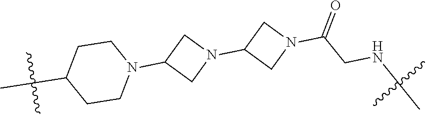 [SMILES: *NCC(=O)N1CC(N2CC(N3CCC(*)CC3)C2)C1]

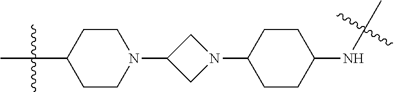 [SMILES: *NC1CCC(N2CC(N3CCC(*)CC3)C2)CC1]

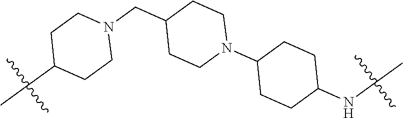 [SMILES: *NC1CCC(N2CCC(CN3CCC(*)CC3)CC2)CC1]

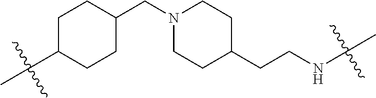 [SMILES: *NCCC1CCN(CC2CCC(*)CC2)CC1]

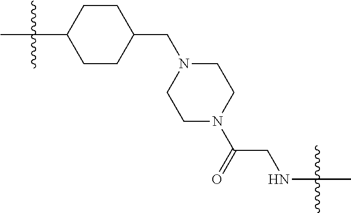 [SMILES: *NCC(=O)N1CCN(CC2CCC(*)CC2)CC1]

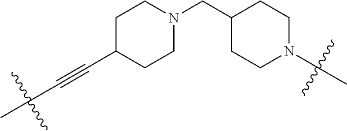 [SMILES: *C#CC1CCN(CC2CCN(*)CC2)CC1]

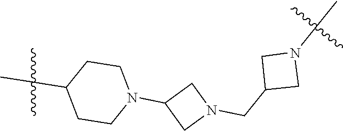 [SMILES: *C1CCN(C2CN(CC3CN(*)C3)C2)CC1]

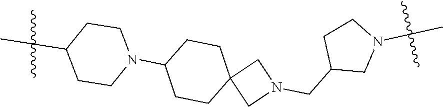 [SMILES: *C1CCN(C2CCC3(CC2)CN(CC2CCN(*)C2)C3)CC1]

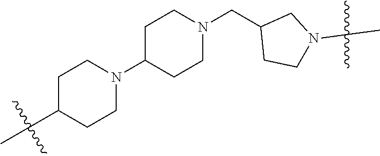 [SMILES: *C1CCN(C2CCN(CC3CCN(*)C3)CC2)CC1]

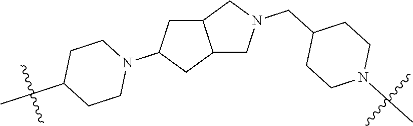 [SMILES: *C1CCN(C2CC3CN(CC4CCN(*)CC4)CC3C2)CC1]

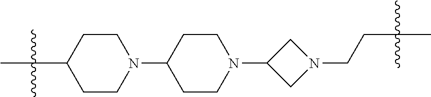 [SMILES: *CCN1CC(N2CCC(N3CCC(*)CC3)CC2)C1]

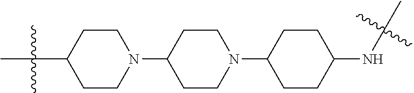 [SMILES: *NC1CCC(N2CCC(N3CCC(*)CC3)CC2)CC1]

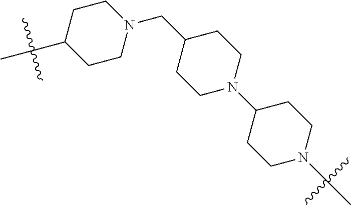 [SMILES: *C1CCN(CC2CCN(C3CCN(*)CC3)CC2)CC1]

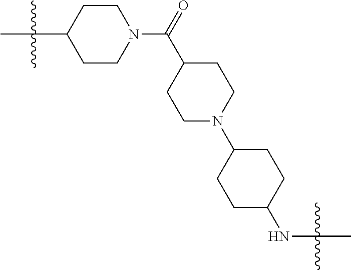 [SMILES: *NC1CCC(N2CCC(C(=O)N3CCC(*)CC3)CC2)CC1]

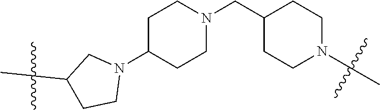 [SMILES: *C1CCN(C2CCN(CC3CCN(*)CC3)CC2)C1]

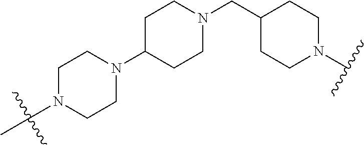 [SMILES: *N1CCC(CN2CCC(N3CCN(*)CC3)CC2)CC1]

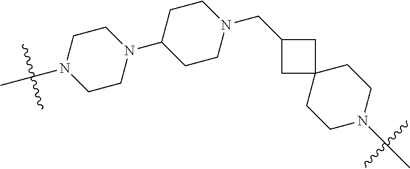 [SMILES: *N1CCN(C2CCN(CC3CC4(CCN(*)CC4)C3)CC2)CC1]

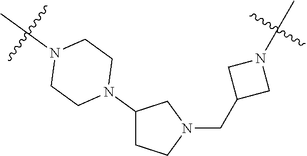 [SMILES: *N1CCN(C2CCN(CC3CN(*)C3)C2)CC1]

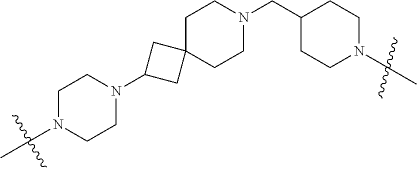 [SMILES: *N1CCC(CN2CCC3(CC2)CC(N2CCN(*)CC2)C3)CC1]

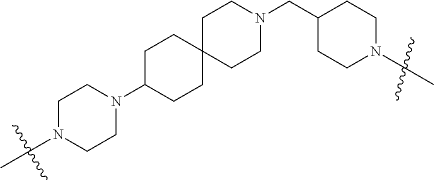 [SMILES: *N1CCC(CN2CCC3(CCC(N4CCN(*)CC4)CC3)CC2)CC1]

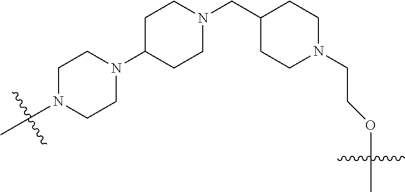 [SMILES: *OCCN1CCC(CN2CCC(N3CCN(*)CC3)CC2)CC1]

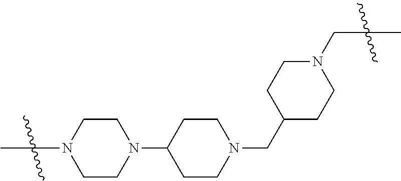 [SMILES: *CN1CCC(CN2CCC(N3CCN(*)CC3)CC2)CC1]

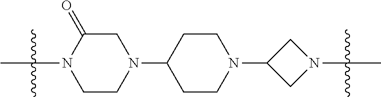 [SMILES: *N1CC(N2CCC(N3CCN(*)C(=O)C3)CC2)C1]

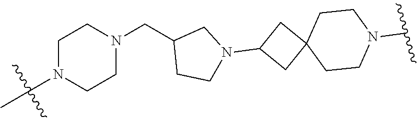 [SMILES: *N1CCN(CC2CCN(C3CC4(CCN(*)CC4)C3)C2)CC1]

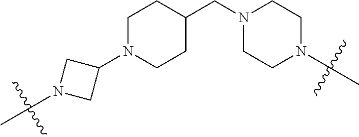 [SMILES: *N1CCN(CC2CCN(C3CN(*)C3)CC2)CC1]

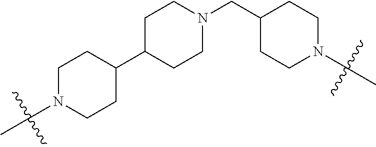 [SMILES: *N1CCC(CN2CCC(C3CCN(*)CC3)CC2)CC1]

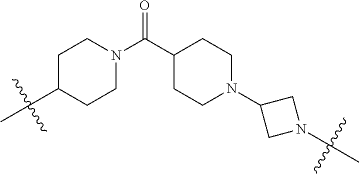 [SMILES: *C1CCN(C(=O)C2CCN(C3CN(*)C3)CC2)CC1]

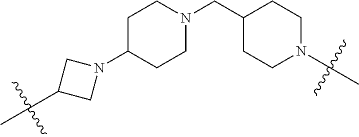 [SMILES: *C1CN(C2CCN(CC3CCN(*)CC3)CC2)C1]

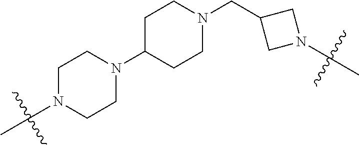 [SMILES: *N1CCN(C2CCN(CC3CN(*)C3)CC2)CC1]

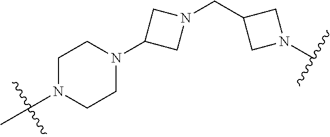 [SMILES: *N1CCN(C2CN(CC3CN(*)C3)C2)CC1]

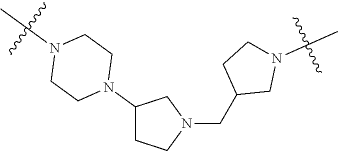 [SMILES: *N1CCN(C2CCN(CC3CCN(*)C3)C2)CC1]

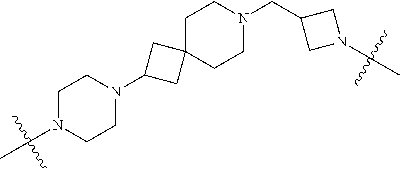 [SMILES: *N1CCN(C2CC3(CCN(CC4CN(*)C4)CC3)C2)CC1]

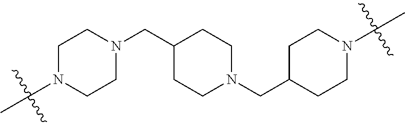 [SMILES: *N1CCC(CN2CCC(CN3CCN(*)CC3)CC2)CC1]

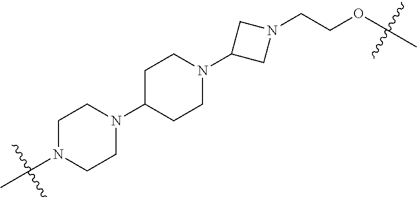 [SMILES: *OCCN1CC(N2CCC(N3CCN(*)CC3)CC2)C1]

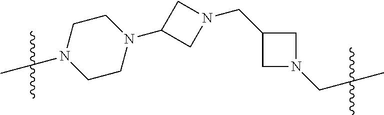 [SMILES: *CN1CC(CN2CC(N3CCN(*)CC3)C2)C1]

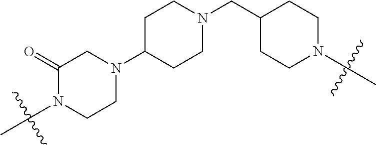 [SMILES: *N1CCC(CN2CCC(N3CCN(*)C(=O)C3)CC2)CC1]

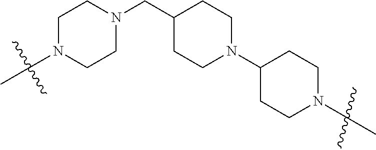 [SMILES: *N1CCC(N2CCC(CN3CCN(*)CC3)CC2)CC1]

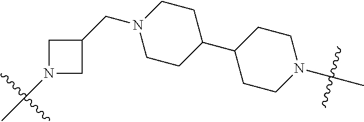 [SMILES: *N1CCC(C2CCN(CC3CN(*)C3)CC2)CC1]

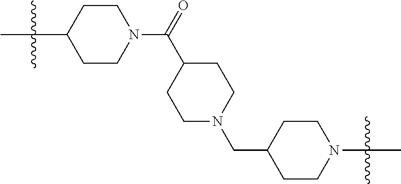 [SMILES: *C1CCN(C(=O)C2CCN(CC3CCN(*)CC3)CC2)CC1]

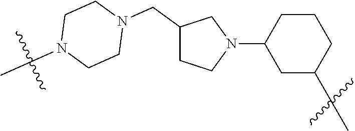 [SMILES: *C1CCCC(N2CCC(CN3CCN(*)CC3)C2)C1]

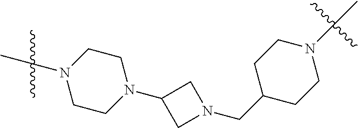 [SMILES: *N1CCC(CN2CC(N3CCN(*)CC3)C2)CC1]

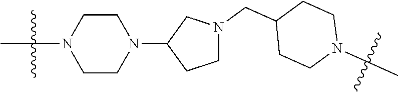 [SMILES: *N1CCC(CN2CCC(N3CCN(*)CC3)C2)CC1]

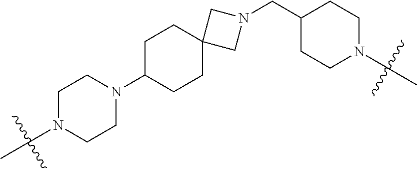 [SMILES: *N1CCC(CN2CC3(CCC(N4CCN(*)CC4)CC3)C2)CC1]

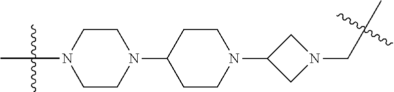 [SMILES: *CN1CC(N2CCC(N3CCN(*)CC3)CC2)C1]

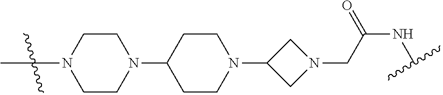 [SMILES: *NC(=O)CN1CC(N2CCC(N3CCN(*)CC3)CC2)C1]

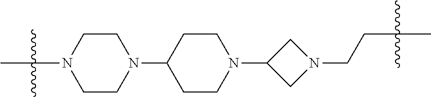 [SMILES: *CCN1CC(N2CCC(N3CCN(*)CC3)CC2)C1]

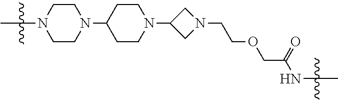 [SMILES: *NC(=O)COCCN1CC(N2CCC(N3CCN(*)CC3)CC2)C1]

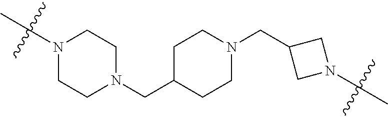 [SMILES: *N1CCN(CC2CCN(CC3CN(*)C3)CC2)CC1]

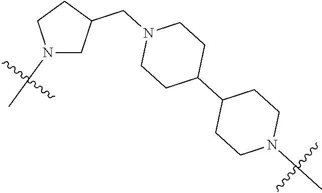 [SMILES: *N1CCC(C2CCN(CC3CCN(*)C3)CC2)CC1]

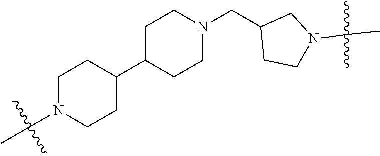 [SMILES: *N1CCC(C2CCN(CC3CCN(*)C3)CC2)CC1]

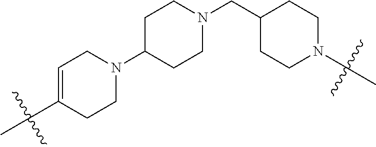 [SMILES: *C1=CCN(C2CCN(CC3CCN(*)CC3)CC2)CC1]

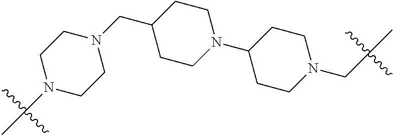 [SMILES: *CN1CCC(N2CCC(CN3CCN(*)CC3)CC2)CC1]

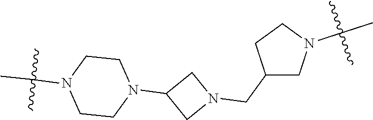 [SMILES: *N1CCN(C2CN(CC3CCN(*)C3)C2)CC1]

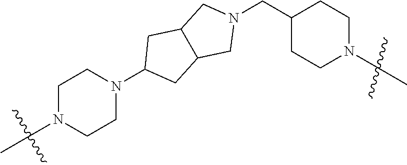 [SMILES: *N1CCC(CN2CC3CC(N4CCN(*)CC4)CC3C2)CC1]

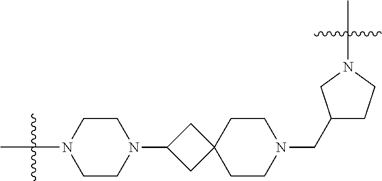 [SMILES: *N1CCN(C2CC3(CCN(CC4CCN(*)C4)CC3)C2)CC1]

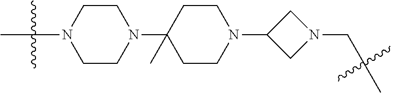 [SMILES: *CN1CC(N2CCC(C)(N3CCN(*)CC3)CC2)C1]

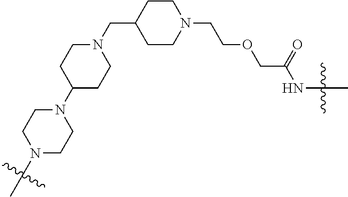 [SMILES: *NC(=O)COCCN1CCC(CN2CCC(N3CCN(*)CC3)CC2)CC1]

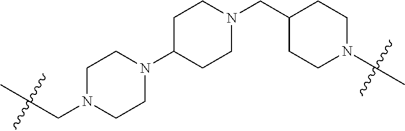 [SMILES: *CN1CCN(C2CCN(CC3CCN(*)CC3)CC2)CC1]

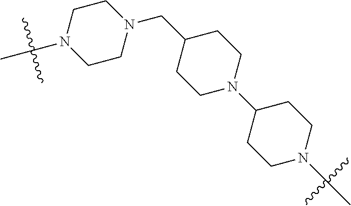 [SMILES: *N1CCC(N2CCC(CN3CCN(*)CC3)CC2)CC1]

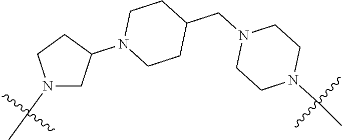 [SMILES: *N1CCN(CC2CCN(C3CCN(*)C3)CC2)CC1]

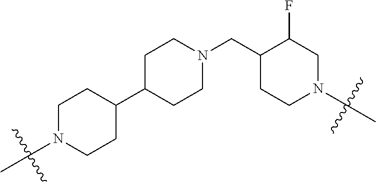 [SMILES: *N1CCC(C2CCN(CC3CCN(*)CC3F)CC2)CC1]

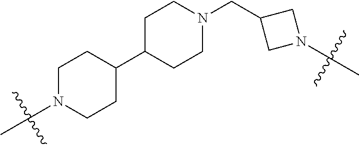 [SMILES: *N1CCC(C2CCN(CC3CN(*)C3)CC2)CC1]

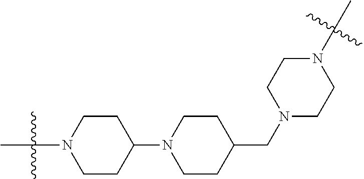 [SMILES: *N1CCC(N2CCC(CN3CCN(*)CC3)CC2)CC1]

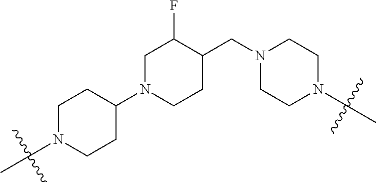 [SMILES: *N1CCC(N2CCC(CN3CCN(*)CC3)C(F)C2)CC1]

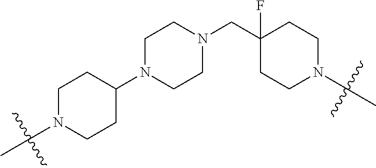 [SMILES: *N1CCC(N2CCN(CC3(F)CCN(*)CC3)CC2)CC1]

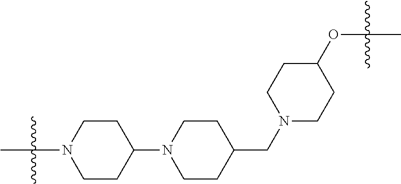 [SMILES: *OC1CCN(CC2CCN(C3CCN(*)CC3)CC2)CC1]

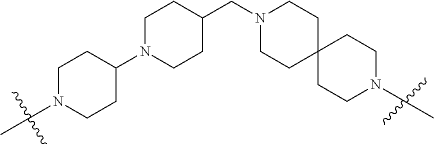 [SMILES: *N1CCC(N2CCC(CN3CCC4(CCN(*)CC4)CC3)CC2)CC1]

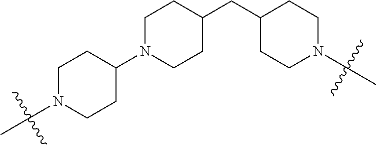 [SMILES: *N1CCC(CC2CCN(C3CCN(*)CC3)CC2)CC1]

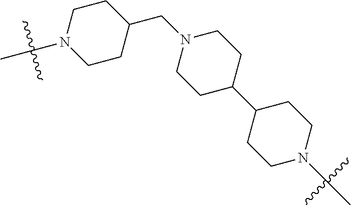 [SMILES: *N1CCC(CN2CCC(C3CCN(*)CC3)CC2)CC1]

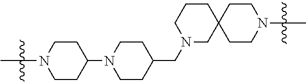 [SMILES: *N1CCC(N2CCC(CN3CCCC4(CCN(*)CC4)C3)CC2)CC1]

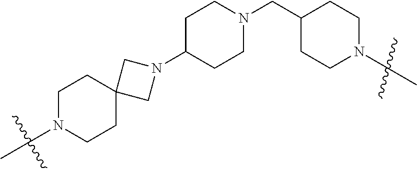 [SMILES: *N1CCC(CN2CCC(N3CC4(CCN(*)CC4)C3)CC2)CC1]

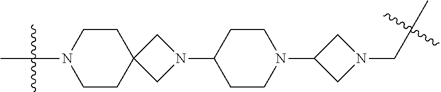 [SMILES: *CN1CC(N2CCC(N3CC4(CCN(*)CC4)C3)CC2)C1]

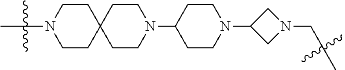 [SMILES: *CN1CC(N2CCC(N3CCC4(CCN(*)CC4)CC3)CC2)C1]

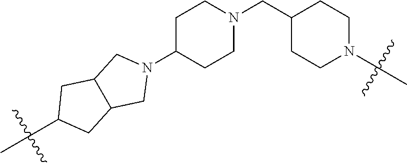 [SMILES: *C1CC2CN(C3CCN(CC4CCN(*)CC4)CC3)CC2C1]

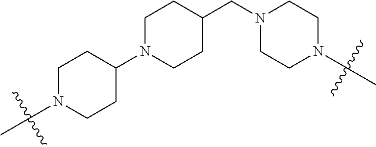 [SMILES: *N1CCC(N2CCC(CN3CCN(*)CC3)CC2)CC1]

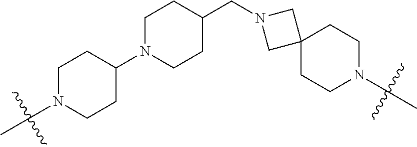 [SMILES: *N1CCC(N2CCC(CN3CC4(CCN(*)CC4)C3)CC2)CC1]

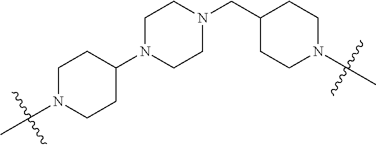 [SMILES: *N1CCC(CN2CCN(C3CCN(*)CC3)CC2)CC1]

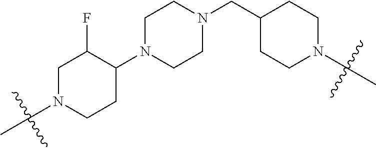 [SMILES: *N1CCC(CN2CCN(C3CCN(*)CC3F)CC2)CC1]

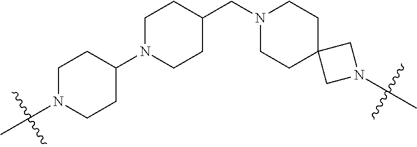 [SMILES: *N1CCC(N2CCC(CN3CCC4(CC3)CN(*)C4)CC2)CC1]

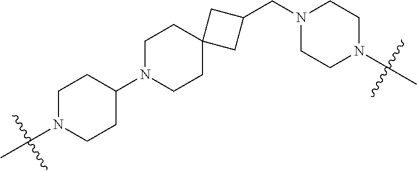 [SMILES: *N1CCC(N2CCC3(CC2)CC(CN2CCN(*)CC2)C3)CC1]

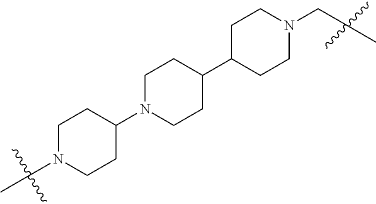 [SMILES: *CN1CCC(C2CCN(C3CCN(*)CC3)CC2)CC1]

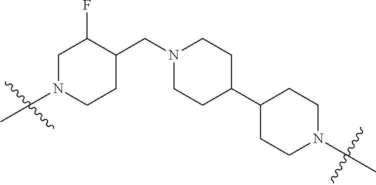 [SMILES: *N1CCC(C2CCN(CC3CCN(*)CC3F)CC2)CC1]

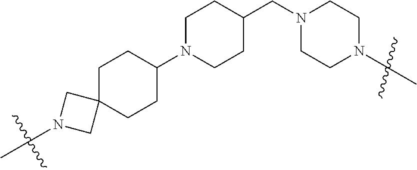 [SMILES: *N1CCN(CC2CCN(C3CCC4(CC3)CN(*)C4)CC2)CC1]

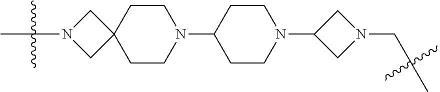 [SMILES: *CN1CC(N2CCC(N3CCC4(CC3)CN(*)C4)CC2)C1]

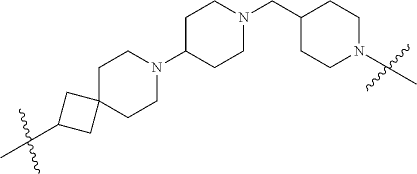 [SMILES: *C1CC2(CCN(C3CCN(CC4CCN(*)CC4)CC3)CC2)C1]

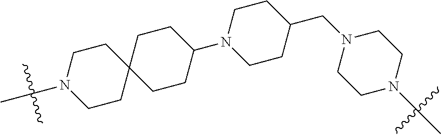 [SMILES: *N1CCN(CC2CCN(C3CCC4(CC3)CCN(*)CC4)CC2)CC1]

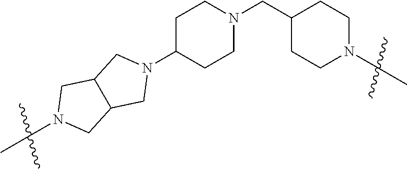 [SMILES: *N1CCC(CN2CCC(N3CC4CN(*)CC4C3)CC2)CC1]

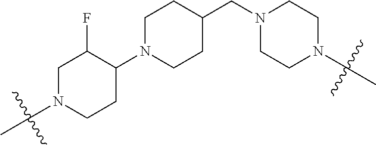 [SMILES: *N1CCN(CC2CCN(C3CCN(*)CC3F)CC2)CC1]

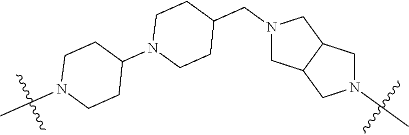 [SMILES: *N1CCC(N2CCC(CN3CC4CN(*)CC4C3)CC2)CC1]

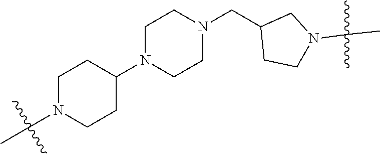 [SMILES: *N1CCC(N2CCN(CC3CCN(*)C3)CC2)CC1]

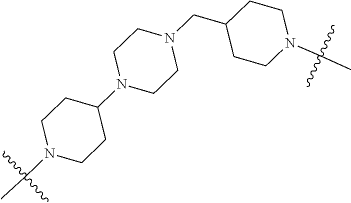 [SMILES: *N1CCC(CN2CCN(C3CCN(*)CC3)CC2)CC1]

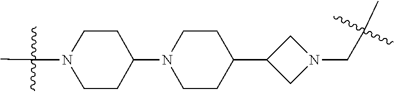 [SMILES: *CN1CC(C2CCN(C3CCN(*)CC3)CC2)C1]

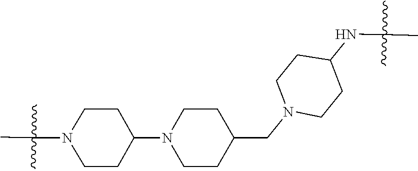 [SMILES: *NC1CCN(CC2CCN(C3CCN(*)CC3)CC2)CC1]

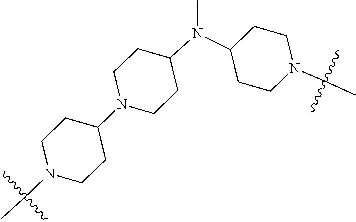 [SMILES: *N1CCC(N2CCC(N(C)C3CCN(*)CC3)CC2)CC1]

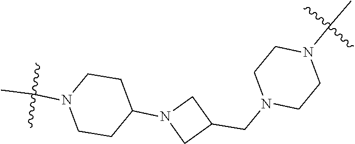 [SMILES: *N1CCC(N2CC(CN3CCN(*)CC3)C2)CC1]

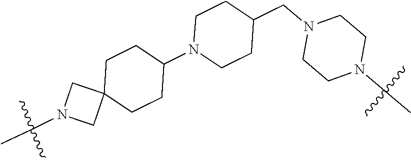 [SMILES: *N1CCN(CC2CCN(C3CCC4(CC3)CN(*)C4)CC2)CC1]

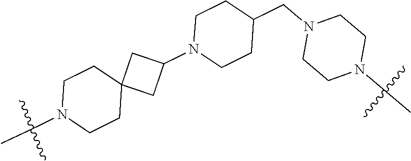 [SMILES: *N1CCN(CC2CCN(C3CC4(CCN(*)CC4)C3)CC2)CC1]

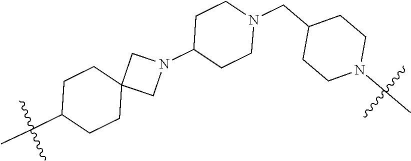 [SMILES: *C1CCC2(CC1)CN(C1CCN(CC3CCN(*)CC3)CC1)C2]

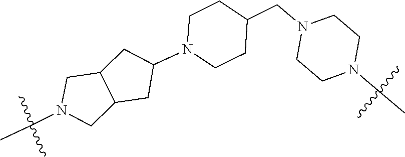 [SMILES: *N1CCN(CC2CCN(C3CC4CN(*)CC4C3)CC2)CC1]

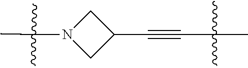 [SMILES: *C#CC1CN(*)C1]

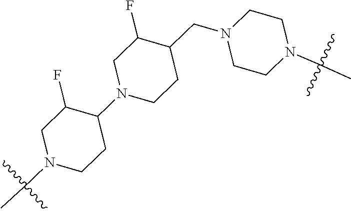 [SMILES: *N1CCN(CC2CCN(C3CCN(*)CC3F)CC2F)CC1]

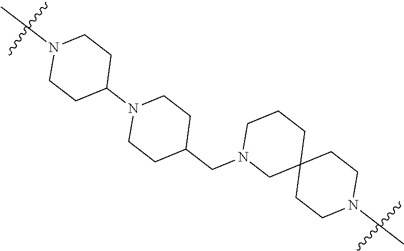 [SMILES: *N1CCC(N2CCC(CN3CCCC4(CCN(*)CC4)C3)CC2)CC1]

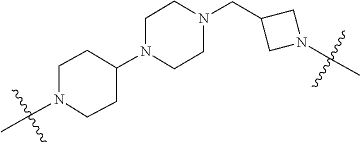 [SMILES: *N1CCC(N2CCN(CC3CN(*)C3)CC2)CC1]

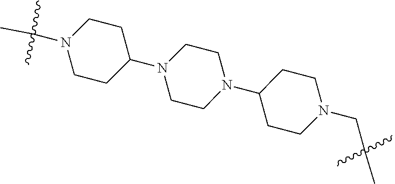 [SMILES: *CN1CCC(N2CCN(C3CCN(*)CC3)CC2)CC1]

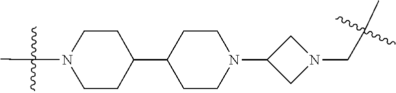 [SMILES: *CN1CC(N2CCC(C3CCN(*)CC3)CC2)C1]

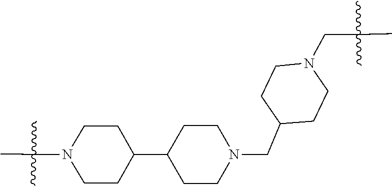 [SMILES: *CN1CCC(CN2CCC(C3CCN(*)CC3)CC2)CC1]

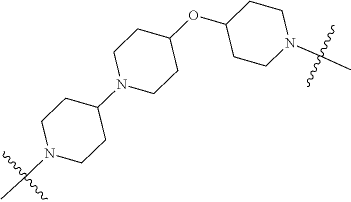 [SMILES: *N1CCC(OC2CCN(C3CCN(*)CC3)CC2)CC1]

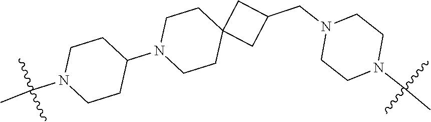 [SMILES: *N1CCC(N2CCC3(CC2)CC(CN2CCN(*)CC2)C3)CC1]

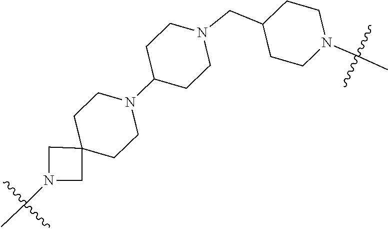 [SMILES: *N1CCC(CN2CCC(N3CCC4(CC3)CN(*)C4)CC2)CC1]

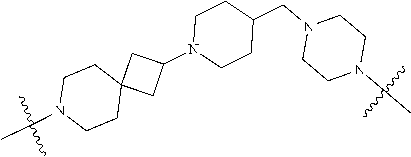 [SMILES: *N1CCN(CC2CCN(C3CC4(CCN(*)CC4)C3)CC2)CC1]

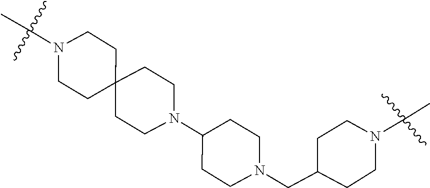 [SMILES: *N1CCC(CN2CCC(N3CCC4(CCN(*)CC4)CC3)CC2)CC1]

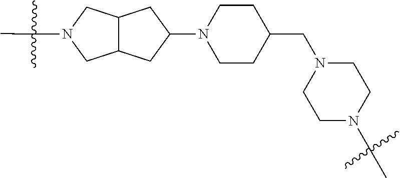 [SMILES: *N1CCN(CC2CCN(C3CC4CN(*)CC4C3)CC2)CC1]

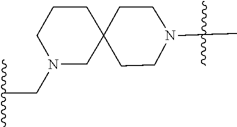 [SMILES: *CN1CCCC2(CCN(*)CC2)C1]

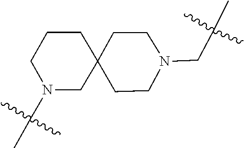 [SMILES: *CN1CCC2(CCCN(*)C2)CC1]

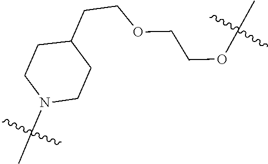 [SMILES: *OCCOCCC1CCN(*)CC1]

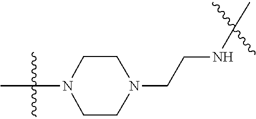 [SMILES: *NCCN1CCN(*)CC1]

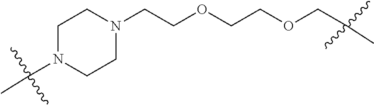 [SMILES: *COCCOCCN1CCN(*)CC1]

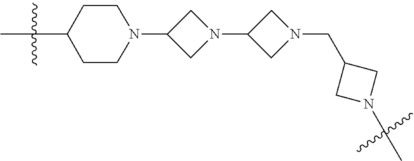 [SMILES: *C1CCN(C2CN(C3CN(CC4CN(*)C4)C3)C2)CC1]

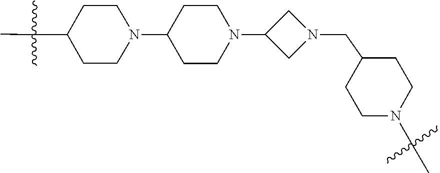 [SMILES: *C1CCN(C2CCN(C3CN(CC4CCN(*)CC4)C3)CC2)CC1]

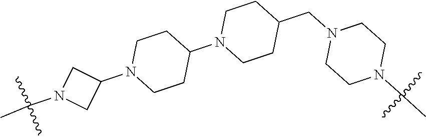 [SMILES: *N1CCN(CC2CCN(C3CCN(C4CN(*)C4)CC3)CC2)CC1]

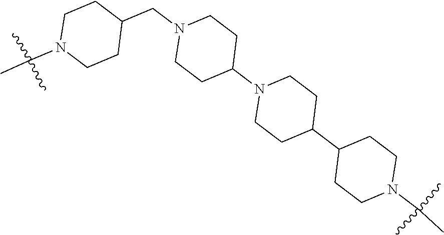 [SMILES: *N1CCC(CN2CCC(N3CCC(C4CCN(*)CC4)CC3)CC2)CC1]

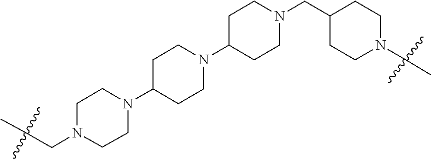 [SMILES: *CN1CCN(C2CCN(C3CCN(CC4CCN(*)CC4)CC3)CC2)CC1]

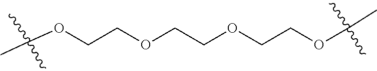 [SMILES: *OCCOCCOCCO*]

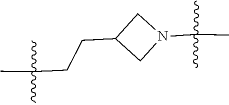 [SMILES: *CCC1CN(*)C1]

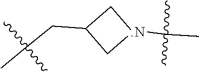

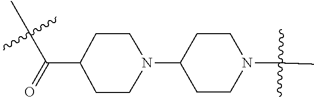 [SMILES: *C(=O)C1CCN(C2CCN(*)CC2)CC1]

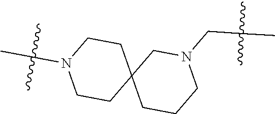 [SMILES: *CN1CCCC2(CCN(*)CC2)C1]

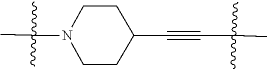 [SMILES: *C#CC1CCN(*)CC1]

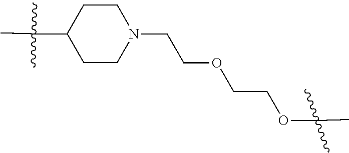 [SMILES: *OCCOCCN1CCC(*)CC1]

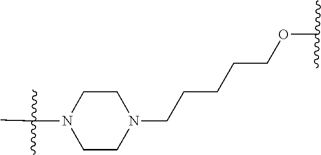 [SMILES: *OCCCCCN1CCN(*)CC1]

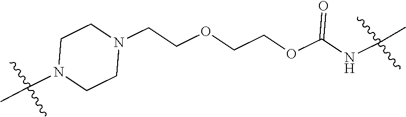 [SMILES: *NC(=O)OCCOCCN1CCN(*)CC1]

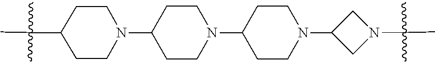 [SMILES: *C1CCN(C2CCN(C3CCN(C4CN(*)C4)CC3)CC2)CC1]

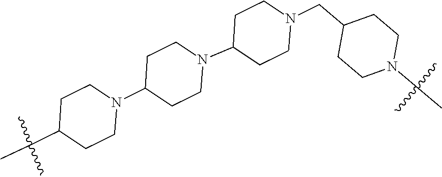 [SMILES: *C1CCN(C2CCN(C3CCN(CC4CCN(*)CC4)CC3)CC2)CC1]

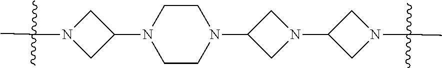 [SMILES: *N1CC(N2CCN(C3CN(C4CN(*)C4)C3)CC2)C1]

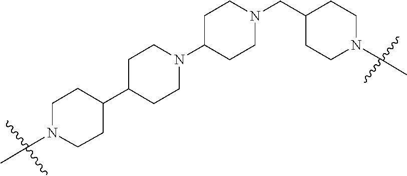 [SMILES: *N1CCC(CN2CCC(N3CCC(C4CCN(*)CC4)CC3)CC2)CC1]

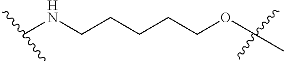 [SMILES: *NCCCCCO*]

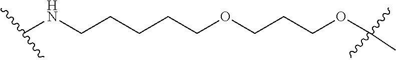 [SMILES: *NCCCCCOCCCO*]

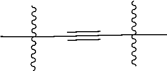 [SMILES: *C#C*]

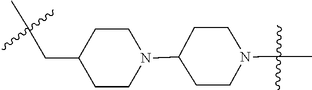 [SMILES: *CC1CCN(C2CCN(*)CC2)CC1]

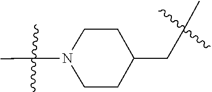 [SMILES: *CC1CCN(*)CC1]

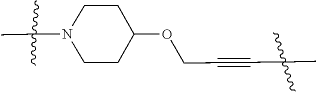 [SMILES: *C#CCOC1CCN(*)CC1]

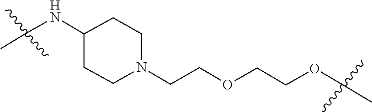 [SMILES: *NC1CCN(CCOCCO*)CC1]

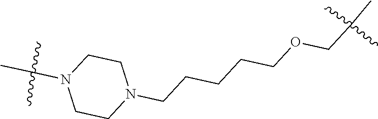 [SMILES: *COCCCCCN1CCN(*)CC1]

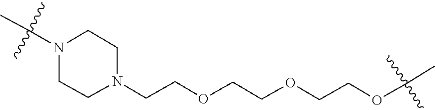 [SMILES: *OCCOCCOCCN1CCN(*)CC1]

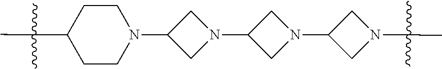 [SMILES: *C1CCN(C2CN(C3CN(C4CN(*)C4)C3)C2)CC1]

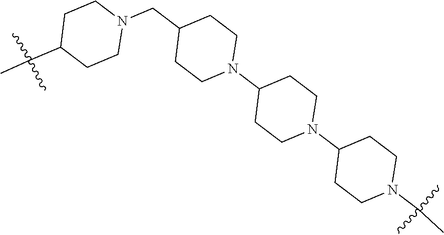 [SMILES: *C1CCN(CC2CCN(C3CCN(C4CCN(*)CC4)CC3)CC2)CC1]

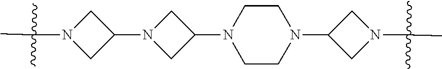 [SMILES: *N1CC(N2CCN(C3CN(C4CN(*)C4)C3)CC2)C1]

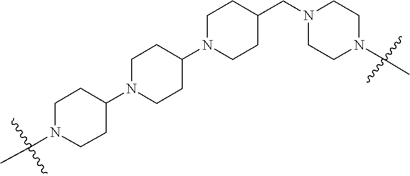 [SMILES: *N1CCC(N2CCC(N3CCC(CN4CCN(*)CC4)CC3)CC2)CC1]

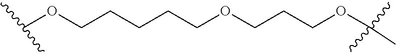 [SMILES: *OCCCCCOCCCO*]

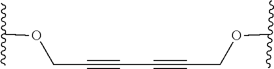 [SMILES: *OCC#CC#CCO*]

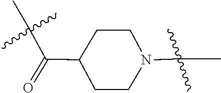 [SMILES: *C(=O)C1CCN(*)CC1]

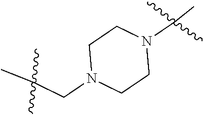 [SMILES: *CN1CCN(*)CC1]

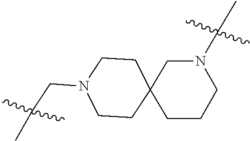 [SMILES: *CN1CCC2(CCCN(*)C2)CC1]

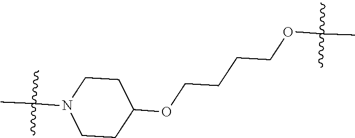 [SMILES: *OCCCCOC1CCN(*)CC1]

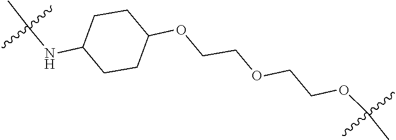 [SMILES: *NC1CCC(OCCOCCO*)CC1]

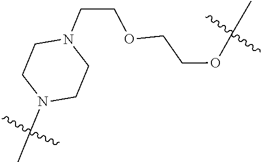 [SMILES: *OCCOCCN1CCN(*)CC1]

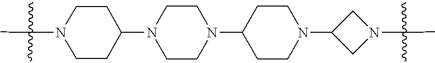 [SMILES: *N1CCC(N2CCN(C3CCN(C4CN(*)C4)CC3)CC2)CC1]

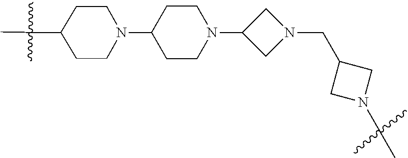 [SMILES: *C1CCN(C2CCN(C3CN(CC4CN(*)C4)C3)CC2)CC1]

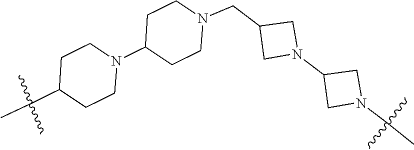 [SMILES: *C1CCN(C2CCN(CC3CN(C4CN(*)C4)C3)CC2)CC1]

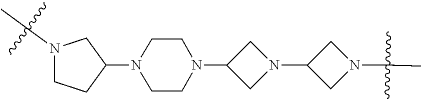 [SMILES: *N1CCC(N2CCN(C3CN(C4CN(*)C4)C3)CC2)C1]

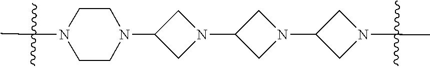 [SMILES: *N1CCN(C2CN(C3CN(C4CN(*)C4)C3)C2)CC1]

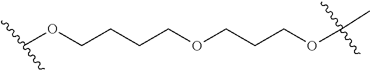 [SMILES: *OCCCCOCCCO*]

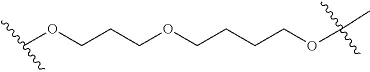 [SMILES: *OCCCCOCCCO*]

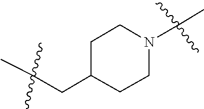 [SMILES: *CC1CCN(*)CC1]

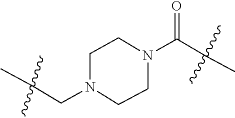 [SMILES: *CN1CCN(C(*)=O)CC1]

—(CH₂)—
—(CH₂)₅—
—(CH₂)₉—
—(CH₂)₄O—
—(CH₂)₂—
—(CH₂)₆—
—(CH₂)O—
—(CH₂)₅O—
—(CH₂)₃—
—(CH₂)₇—
—(CH₂)₂O—
—(CH₂)₆O—
—(CH₂)₄—
—(CH₂)₈—
—(CH₂)₃O—
—(CH₂)₇O—
—(CH₂)₈O—
—(CH₂)₄OCH₂—
—(CH₂)₈OCH₂—
—(CH₂)₄OCH₂CH₂—
—(CH₂)₈OCH₂CH₂—
—(CH₂CH₂O)₅—
—(CH₂CH₂O)₂O—
—(CH₂)NH—
—(CH₂)₅NH—
—(CH₂)NHCH₂—
—(CH₂)₅NHCH₂—
—(CH₂)NHCH₂CH₂—
—(CH₂)₅NHCH₂CH₂—
—(CH₂)N(CH₃)—
—(CH₂)₅N(CH₃)—
—(CH₂)N(CH₃)CH₂—
—(CH₂)₅N(CH₃)CH₂—
—C(═O)—
—C(═O)(CH₂)₄—
—C(═O)(CH₂)₈—
—C(═O)CH₂CH₂((CH₂)₂—
—C(═O)CH₂OCH₂—
—C(═O)(CH₂)₂O—
—C(═O)(CH₂)₆O—
—C(═O)(CH₂)₄OCH₂—
—C(═O)CH₂CH₂NH—
—C(═O)(CH₂)₂NH(CH₂)₃—
—C(═O)CH₂NHCH₂—
—C(═O)CH₂NH(CH₂)₄—
—C(═O)(CH₂)₄NH—
—(CH₂)OCH₂—
—(CH₂)₅OCH₂—
—(CH₂)OCH₂CH₂—
—(CH₂)₅OCH₂CH₂—
—(CH₂CH₂O)₂—
—(CH₂CH₂O)₆—
—(CH₂CH₂O)₃O—
—(CH₂)₂NH—
—(CH₂)₆NH—
—(CH₂)₂NHCH₂—
—(CH₂)₆NHCH₂—
—(CH₂)₂NHCH₂CH₂—
—(CH₂)₆NHCH₂CH₂—
—(CH₂)₂N(CH₃)—
—(CH₂)₆N(CH₃)—
—(CH₂)₂N(CH₃)CH₂—
—(CH₂)₆N(CH₃)CH₂—
—C(═O)CH₂—
—C(═O)(CH₂)₅—
—C(═O)(CH₂)₉—
—C(═O)CH₂CH₂O(CH₂)₃—
—C(═O)CH₂O(CH₂)₂—
—C(═O)(CH₂)₃O—
—C(═O)(CH₂)₂OCH₂—
—C(═O)(CH₂)₅OCH₂—
—C(═O)(CH₂)₂NHCH₂—
—C(═O)(CH₂)₂NH(CH₂)₄—
—C(═O)CH₂NH(CH₂)₂—
—C(═O)(CH₂)₂NH—
—C(═O)(CH₂)₅NH—
—(CH₂)₂OCH₂—
—(CH₂)₆OCH₂—
—(CH₂)₂OCH₂CH₂—
—(CH₂)₆OCH₂CH₂—
—(CH₂CH₂O)₃—
—(CH₂CH₂O)₇—
—(CH₂CH₂O)₄O—
—(CH₂)₃ NH—
—(CH₂)₇NH—
—(CH₂)₃NHCH₂—
—(CH₂)—NHCH₂—
—(CH₂)₃ NHCH₂CH₂—
—(CH₂)₇NHCH₂CH₂—
—(CH₂)₃N(CH₃)—
—(CH₂)₇NH(CH₃)—
—(CH₂)₃N(CH₃)CH₂—
—(CH₂)₇N(CH₃)CH₂—
—C(═O)CH₂CH₂—
—C(═O)(CH₂)₆—
—C(═O)CH₂CH₂O—
—C(═O)(CH₂)₂O(CH₂)₄—
—C(═O)CH₂O(CH₂)₃—
—C(═O)(CH₂)₄O—
—C(═O)(CH₂)₃OCH₂—
—C(═O)(CH₂)₆OCH₂—
—C(═O)(CH₂)₂NH(CH₂)₂—
—C(═O)CH₂NH—
—C(═O)CH₂NH(CH₂)₃—
—C(═O)(CH₂)₃NH—
—C(═O)(CH₂)₆NH—
—(CH₂)₇OCH₂—
—(CH₂)₃OCH₂CH₂—
—(CH₂)₇OCH₂CH₂—
—(CH₂CH₂O)₄—
—(CH₂CH₂O)₈—
—(CH₂)₄NH—
—(CH₂)₈NH—
—(CH₂)₄NHCH₂—
—(CH₂)₈NHCH₂—
—(CH₂)₄NHCH₂CH₂—
—(CH₂)₈NHCH₂CH₂—
—(CH₂)₄N(CH₃)—
—(CH₂)₈N(CH₃)—
—(CH₂)₄N(CH₃)CH₂—
—(CH₂)₈N(CH₃)CH₂—
—C(═O)(CH₂)₃—
—C(═O)(CH₂)₇—
—C(═O)CH₂CH₂OCH₂—
—C(═O)CH₂O—
—C(═O)CH₂O(CH₂)₄—
—C(═O)(CH₂)₅O—
—C(═O)(CH₂)₂NHCH₂—
—C(═O)(CH₂)₅NHCH₂—
—C(═O)CH₂(O(CH₂)₂)₂O—
—C(═O)CH₂O(CH₂)₂—
—C(O)CH₂(O(CH₂)₂)₄—
—C(O)(CH₂)₂(O(CH₂)₂)₃—
—C(═O)CH₂O(CH₂)₂O—
—C(═O)CH₂O(CH₂)₅O—
—C(═O)CH₂O(CH₂)₆OCH₂—
—C(═O)CH₂O(CH₂)₄OCH₂—
—C(═O)CH₂O(CH₂)₂OCH₂—
—C(═O)CH₂O(CH₂)₂O(CH₂)₃—
—C(═O)(CH₂)OCH₂CH₂O—
—C(═O)(CH₂)₃OCH₂CH₂O—
—C(═O)CH₂OCH₂CH₂NHCH₂CH₂O—
—C(═O)CH₂OCH₂CH₂OCH₂CH₂NH—
—C(═O)CH₂NHCH₂CH₂OCH₂CH₂O—
—C(═O)CH₂NHCH₂CH₂OCH₂CH₂NH—
—C(═O)CH₂OCH₂CH₂OCH₂CH₂CH₂—
—C(═O)(CH₂)₃NHCH₂—
—C(═O)(CH₂)₆NHCH₂—
—C(═O)CH₂(O(CH₂)₂)₃O—
—C(═O)CH₂(O(CH₂)₂)₂—
—C(═O)(CH₂)₂O(CH₂)₂—
—C(═O)CH₂O(CH₂)₃O—
—C(═O)CH₂O(CH₂)₆O—
—C(═O)(CH₂)₄NHCH₂—
—C(═O)CH₂O(CH₂)₂O—
—C(O)CH₂(O(CH₂)₂)₄O—
—C(═O)CH₂(O(CH₂)₂)₃—
—C(O)(CH₂)₂(O(CH₂)₂)₂—
—C(═O)CH₂OCH₂O—
—C(═O)CH₂O(CH₂)₄O—
—C(═O)CH₂O(CH₂)₇O—
—C(═O)CH₂O(CH₂)₅OCH₂—
—C(═O)CH₂O(CH₂)₃OCH₂—
—C(═O)CH₂O(CH₂)₂O(CH₂)₂—
—C(═O)CH₂O(CH₂)₂O(CH₂)₄—
—C(═O)(CH₂)₂OCH₂CH₂O—
—C(═O)(CH₂)₄OCH₂CH₂O—
—C(═O)CH₂OCH₂CH₂N(CH₃)CH₂CH₂O—
—C(═O)CH₂OCH₂CH₂OCH₂CH₂N(CH₃)—
—C(═O)CH₂N(CH₃)CH₂CH₂OCH₂CH₂O—
—C(═O)CH₂N(CH₃)CH₂CH₂OCH₂CH₂NH—
—C(═O)CH₂OCH₂CH₂NHCH₂CH₂CH₂—

The present invention relates to a pharmaceutical composition, comprising the above-mentioned compound or the stereoisomer, deuterated compound, solvate, prodrug, metabolite, pharmaceutically acceptable salt or co-crystal thereof according to the present invention, and a pharmaceutically acceptable carrier.

The present invention relates to the use of the above-mentioned compound or the stereoisomer, deuterated compound, solvate, prodrug, metabolite, pharmaceutically acceptable salt or co-crystal thereof according to the present invention in the preparation of a medicament for treating a disease related to Bcl-2 family protein activity or expression level.

The present invention relates to the use of the above-mentioned compound or the stereoisomer, deuterated compound, solvate, prodrug, metabolite, pharmaceutically acceptable salt or co-crystal thereof according to the present invention in the preparation of a medicament for treating a disease related to the inhibition or degradation of Bcl-2 family proteins.

The present invention relates to the use of the above-mentioned compound or the stereoisomer, deuterated compound, solvate, prodrug, metabolite, pharmaceutically acceptable salt or co-crystal thereof according to the present invention, wherein the disease is selected from cancer.

The present invention relates to a pharmaceutical composition or pharmaceutical preparation comprising a therapeutically effective amount of the compound, or the stereoisomer, deuterated compound, solvate, prodrug, metabolite, pharmaceutically acceptable salt or co-crystal thereof according to the present invention, and a pharmaceutically acceptable excipient. The pharmaceutical composition can be in a unit preparation form (the amount of the active drug in the unit preparation is also referred to as the “preparation specification”).

The present invention further provides a method for treating a disease in a mammal, the method comprising administering to the mammal a therapeutically effective amount of the compound, or the stereoisomer, deuterated compound, solvate, prodrug, metabolite, pharmaceutically acceptable salt or co-crystal thereof or the pharmaceutical composition according to the present invention. In some embodiments, the mammal according to the present invention comprises humans.

The term “effective amount” or “therapeutically effective amount” according to the present application refers to a sufficient amount of the compound disclosed in the present application that is administered to ameliorate, to some extent, one or more symptoms of a disease or condition (such as cancer) being treated. In some embodiments, the outcome is the reduction and/or remission of signs, symptoms or causes of the disease, or any other desired change in the biological system. For example, an “effective amount” in terms of the therapeutic use is an amount of the composition comprising the compound disclosed in the present application that is required to provide clinically significant reduction of the symptoms of the disease. Examples of the therapeutically effective amount include, but are limited to 1-1500 mg, 1-1000 mg, 1-900 mg, 1-800 mg, 1-700 mg, 1-600 mg, 2-600 mg, 3-600 mg, 4-600 mg, 5-600 mg, 6-600 mg, 10-600 mg, 20-600 mg, 25-600 mg, 30-600 mg, 40-600 mg, 50-600 mg, 60-600 mg, 70-600 mg, 75-600 mg, 80-600 mg, 90-600 mg, 100-600 mg, 200-600 mg, 1-500 mg, 2-500 mg, 3-500 mg, 4-500 mg, 5-500 mg, 6-500 mg, 10-500 mg, 20-500 mg, 25-500 mg, 30-500 mg, 40-500 mg, 50-500 mg, 60-500 mg, 70-500 mg, 75-500 mg, 80-500 mg, 90-500 mg, 100-500 mg, 125-500 mg, 150-500 mg, 200-500 mg, 250-500 mg, 300-500 mg, 400-500 mg, 5-400 mg, 10-400 mg, 20-400 mg, 25-400 mg, 30-400 mg, 40-400 mg, 50-400 mg, 60-400 mg, 70-400 mg, 75-400 mg, 80-400 mg, 90-400 mg, 100-400 mg, 125-400 mg, 150-400 mg, 200-400 mg, 250-400 mg, 300-400 mg, 1-300 mg, 2-300 mg, 5-300 mg, 10-300 mg, 20-300 mg, 25-300 mg, 30-300 mg, 40-300 mg, 50-300 mg, 60-300 mg, 70-300 mg, 75-300 mg, 80-300 mg, 90-300 mg, 100-300 mg, 125-300 mg, 150-300 mg, 200-300 mg, 250-300 mg, 1-200 mg, 2-200 mg, 5-200 mg, 10-200 mg, 20-200 mg, 25-200 mg, 30-200 mg, 40-200 mg, 50-200 mg, 60-200 mg, 70-200 mg, 75-200 mg, 80-200 mg, 90-200 mg, 100-200 mg, 125-200 mg, 150-200 mg, 80-1500 mg, 80-1000 mg, and 80-800 mg;
in some embodiments, the pharmaceutical composition comprises the compound, or the stereoisomer, deuterated compound, solvate, prodrug, metabolite, pharmaceutically acceptable salt or co-crystal thereof according to the present invention in an amount including but not limited to 1-1500 mg, 1-1000 mg, 20-800 mg, 40-800 mg, 40-400 mg, 25-200 mg, 1 mg, 5 mg, 10 mg, 15 mg, 20 mg, 25 mg, 30 mg, 35 mg, 40 mg, 45 mg, 50 mg, 55 mg, 65 mg, 70 mg, 75 mg, 80 mg, 85 mg, 90 mg, 95 mg, 100 mg, 110 mg, 120 mg, 125 mg, 130 mg, 140 mg, 150 mg, 160 mg, 170 mg, 180 mg, 190 mg, 200 mg, 210 mg, 220 mg, 230 mg, 240 mg, 250 mg, 300 mg, 320 mg, 400 mg, 480 mg, 500 mg, 600 mg, 640 mg, 840 mg, and 1000 mg.

The present invention further provides a method for treating a disease in a mammal, the method comprising administering to a subject a therapeutically effective amount of the compound, or the stereoisomer, deuterated compound, solvate, prodrug, metabolite, pharmaceutically acceptable salt, or co-crystal thereof according to the present invention, wherein the therapeutically effective amount is preferably 1-1500 mg, and the disease is preferably cancer.

The present invention further provides a method for treating a disease in a mammal, the method comprising administering to a subject a medicament, i.e., the compound, or the stereoisomer, deuterated compound, solvate, prodrug, metabolite, pharmaceutically acceptable salt, or co-crystal thereof according to the present invention in a daily dose of 1-1500 mg/day, wherein the daily dose can be a single dose or divided doses; in some embodiments, the daily dose includes, but is not limited to 10-1500 mg/day, 10-1000 mg/day, 10-800 mg/day, 25-800 mg/day, 50-800 mg/day, 100-800 mg/day, 200-800 mg/day, 25-400 mg/day, 50-400 mg/day, 100-400 mg/day, or 200-400 mg/day; in some embodiments, the daily dose includes, but is not limited to 10 mg/day, 20 mg/day, 25 mg/day, 50 mg/day, 80 mg/day, 100 mg/day, 125 mg/day, 150 mg/day, 160 mg/day, 200 mg/day, 300 mg/day, 320 mg/day, 400 mg/day, 480 mg/day, 600 mg/day, 640 mg/day, 800 mg/day, 1000 mg/day, or 1500 mg/day.

The present invention relates to a kit, wherein the kit can comprise a composition in the form of a single dose or multiple doses and comprises the compound, or the stereoisomer, deuterated compound, solvate, prodrug, metabolite, pharmaceutically acceptable salt, or co-crystal thereof according to the present invention, and the amount of the compound or the stereoisomer, deuterated compound, solvate, prodrug, metabolite, pharmaceutically acceptable salt, or co-crystal thereof according to the present invention is identical to the amount of same in the above-mentioned pharmaceutical composition.

In the present invention, the amount of the compound, or the stereoisomer, deuterated compound, solvate, prodrug, metabolite, pharmaceutically acceptable salt or co-crystal thereof according to the present invention is calculated in the form of a free base in each case.

Unless stated to the contrary, the terms used in the description and claims have the following meanings.

The carbon, hydrogen, oxygen, sulfur, nitrogen or F, Cl, Br, I involved in the groups and compounds of the present invention all comprise their isotopes, and the carbon, hydrogen, oxygen, sulfur or nitrogen involved in the groups and compounds of the present invention is optionally further substituted with one or more of their corresponding isotopes, wherein the isotopes of carbon comprise ¹²C, ¹³C and ¹⁴C, the isotopes of hydrogen comprise protium (H), deuterium (D, also known as heavy hydrogen), tritium (T, also known as superheavy hydrogen), the isotopes of oxygen comprise ¹⁶O, ¹⁷O and ¹⁸O, the isotopes of sulfur comprise ³²S, ³³S, ³⁴S and ³⁶S, the isotopes of nitrogen comprise ¹⁴N and ¹⁵N, the isotopes of fluorine comprise ¹⁷F and ¹⁹F, the isotopes of chlorine comprise ³⁵Cl and ³⁷Cl, and the isotopes of bromine comprise ⁷⁹Br and ⁸¹Br.

“Halogen” refers to F, Cl, Br or I.

“Halogen-substituted” refers to F, Cl, Br or I substitution, including but not limited to a substitution with 1 to 10 substituents selected from F, Cl, Br or I, a substitution with 1 to 6 substituents selected from F, Cl, Br or I, or a substitution with 1 to 4 substituents selected from F, Cl, Br or I. “Halogen-substituted” is referred to simply as “halo”.

“Alkyl” refers to a substituted or unsubstituted linear or branched saturated aliphatic hydrocarbyl group, including but not limited to an alkyl group of 1 to 20 carbon atoms, an alkyl group of 1 to 8 carbon atoms, an alkyl group of 1 to 6 carbon atoms, or an alkyl group of 1 to 4 carbon atoms. Non-limiting examples include methyl, ethyl, n-propyl, isopropyl, n-butyl, sec-butyl, neobutyl, tert-butyl, n-pentyl, isoamyl, neopentyl, n-hexyl and various branched isomers thereof. The definition of the “alkyl” herein is consistent with this definition. Alkyl can be monovalent, divalent, trivalent or tetravalent.

“Hydrocarbyl” refers to a substituted or unsubstituted linear or branched saturated or unsaturated group consisting of carbon and hydrogen atoms. Hydrocarbyl can be monovalent, divalent, trivalent or tetravalent.

“Heteroalkyl” refers to a substituted or unsubstituted alkyl group in which one or more (including but not limited to 2, 3, 4, 5 or 6) carbon atoms are replaced by heteroatoms (including but not limited to N, O or S). Non-limiting examples include —X(CH₂)v-X(CH₂)v-X(CH₂)v-H (v is an integer from 1 to 5; each X is independently selected from a bond or a heteroatom, which includes but is not limited to N, O or S; at least one X is selected from a heteroatom; and N or S in the heteroatom can be oxidized to various oxidation states). Heteroalkyl can be monovalent, divalent, trivalent or tetravalent.

“Alkylene” refers to a substituted or unsubstituted linear or branched divalent saturated hydrocarbyl group, including —(CH₂)ᵥ— (v is an integer from 1 to 10), and examples of alkylene include, but are not limited to, methylene, ethylene, propylene, butylene, etc.

“Heteroalkylene” refers to a substituted or unsubstituted alkylene group in which one or more (including but not limited to 2, 3, 4, 5 or 6) carbon atoms are replaced by heteroatoms (including but not limited to N, O or S). Non-limiting examples include —X(CH₂)v-X(CH₂)v-X(CH₂)v-, wherein v is an integer from 1 to 5, each X is independently selected from a bond, N, O or S, and at least one X is selected from N, O or S.

“Cycloalkyl” refers to a substituted or unsubstituted saturated carbocyclic hydrocarbyl group, usually having from 3 to 10 carbon atoms, and non-limiting examples include cyclopropyl, cyclobutyl, cyclopentyl, cyclohexyl, cycloheptyl, etc. The “cycloalkyl” herein is as defined above. Cycloalkyl can be monovalent, divalent, trivalent or tetravalent.

“Heterocycloalkyl” refers to a substituted or unsubstituted saturated heteroatom-containing cyclic hydrocarbyl group, including but not limited to 3 to 10 atoms, 3 to 8 atoms, or 1 to 3 heteroatoms selected from N, O or S. N and S selectively substituted in the heterocycloalkyl ring can be oxidized to various oxidation states. Heterocycloalkyl can be connected to a heteroatom or a carbon atom; heterocycloalkyl can be connected to an aromatic ring or a non-aromatic ring; and heterocycloalkyl can be connected to a bridged ring or a spiro ring. Non-limiting examples include oxiranyl, azacyclopropyl, oxetanyl, azetidinyl, tetrahydrofuryl, tetrahydro-2H-pyranyl, dioxolanyl, dioxanyl, pyrrolidinyl, piperidyl, imidazolidinyl, oxazolidinyl, oxazinanyl, morpholinyl, hexahydropyrimidyl or piperazinyl. Heterocycloalkyl can be monovalent, divalent, trivalent or tetravalent.

“Alkenyl” refers to a substituted or unsubstituted linear or branched unsaturated hydrocarbyl group, having at least 1, usually 1, 2 or 3 carbon-carbon double bonds, with a main chain including but not limited to 2 to 10, 2 to 6, or 2 to 4 carbon atoms. Examples of alkenyl include, but are not limited to, ethenyl, allyl, 1-propenyl, 2-propenyl, 1-butenyl, 2-butenyl, 3-butenyl, 1-pentenyl, 2-pentenyl, 3-pentenyl, 4-pentenyl, 1-methyl-1-butenyl, 2-methyl-1-butenyl, 2-methyl-3-butenyl, 1-hexenyl, 2-hexenyl, 3-hexenyl, 4-hexenyl, 5-hexenyl, 1-methyl-1-pentenyl, 2-methyl-1-pentenyl, 1-heptenyl, 2-heptenyl, 3-heptenyl, 4-heptenyl, 1-octenyl, 3-octenyl, 1-nonenyl, 3-nonenyl, 1-decenyl, 4-decenyl, 1,3-butadiene, 1,3-pentadiene, 1,4-pentadiene, 1,4-hexadiene, etc. The definition of the “alkenyl” herein is consistent with this definition. Alkenyl can be monovalent, divalent, trivalent or tetravalent.

“Alkynyl” refers to a substituted or unsubstituted linear or branched monovalent unsaturated hydrocarbyl group, having at least 1, usually 1, 2 or 3 carbon-carbon triple bonds, with a main chain including but not limited to 2 to 10 carbon atoms, 2 to 6 carbon atoms, or 2 to 4 carbon atoms. Examples of alkynyl include, but are not limited to, ethynyl, propargyl, 1-propynyl, 2-propynyl, 1-butynyl, 2-butynyl, 3-butynyl, 1-pentynyl, 2-pentynyl, 3-pentynyl, 4-pentynyl, 1-methyl-1-butynyl, 2-methyl-1-butynyl, 2-methyl-3-butynyl, 1-hexynyl, 2-hexynyl, 3-hexynyl, 4-hexynyl, 5-hexynyl, 1-methyl-1-pentynyl, 2-methyl-1-pentynyl, 1-heptynyl, 2-heptynyl, 3-heptynyl, 4-heptynyl, 1-octynyl, 3-octynyl, 1-nonynyl, 3-nonynyl, 1-decynyl, 4-decynyl, etc. Alkynyl can be monovalent, divalent, trivalent or tetravalent.

“Alkoxy” refers to a substituted or unsubstituted —O-alkyl group. Non-limiting examples include methoxy, ethoxy, n-propoxy, isopropoxy, n-butoxy, sec-butoxy, tert-butoxy, n-pentoxy, n-hexyloxy, cyclopropoxy and cyclobutoxy.

“Carbocyclyl” or “carbocycle” refers to a substituted or unsubstituted saturated or unsaturated aromatic ring or non-aromatic ring, wherein the aromatic ring or non-aromatic ring can be a 3- to 8-membered monocyclic ring, a 4- to 12-membered bicyclic ring or a 10- to 15-membered tricyclic ring system. Carbocyclyl can be connected to an aromatic ring or a non-aromatic ring, wherein the aromatic ring or non-aromatic ring is optionally a monocyclic ring, a bridged ring or a spiro ring. Non-limiting examples include cyclopropane, cyclobutane, cyclopentane, cyclohexane, cycloheptane, 1-cyclopentyl-1-enyl, 1-cyclopentyl-2-enyl, 1-cyclopentyl-3-enyl, cyclohexyl, 1-cyclohexyl-2-enyl, 1-cyclohexyl-3-enyl, cyclohexenyl, a benzene ring, a naphthalene ring,

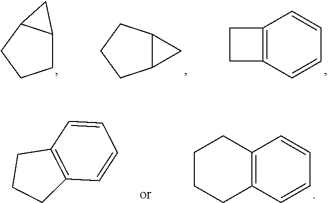 [SMILES: C1CC2CC2C1 ; C1CC2CC2C1 ; c1ccc2c(c1)CC2 ; c1ccc2c(c1)CCC2 ; c1ccc2c(c1)CCCC2]

“Carbocyclyl” or “carbocycle” can be monovalent, divalent, trivalent or tetravalent.

“Heterocyclyl” or “heterocycle” refers to a substituted or unsubstituted saturated or unsaturated aromatic ring or non-aromatic ring, wherein the aromatic ring or non-aromatic ring can be 3- to 8-membered monocyclic ring, 4- to 12-membered bicyclic ring or 10- to 15-membered tricyclic ring system, and contains one or more (including but not limited to 2, 3, 4 or 5) heteroatoms selected from N, O or S, and the selectively substituted N and S in the heterocyclyl ring can be oxidized to various oxidation states. Heterocyclyl can be connected to a heteroatom or a carbon atom; heterocyclyl can be connected to an aromatic ring or a non-aromatic ring; and heterocyclyl can be connected to a bridged ring or a spiro ring. Non-limiting examples include oxiranyl, azacyclopropyl, oxetanyl, azetidinyl, 1,3-dioxolanyl, 1,4-dioxolanyl, 1,3-dioxanyl, azacycloheptyl, pyridyl, furyl, thienyl, pyranyl, N-alkylpyrrolyl, pyrimidyl, pyrazinyl, pyridazinyl, imidazolyl, piperidyl, morpholinyl, thiomorpholinyl, 1,3-dithianyl, dihydrofuryl, dihydropyranyl, dithiolanyl, tetrahydrofuryl, tetrahydropyrrolyl, tetrahydroimidazolyl, tetrahydrothiazolyl, tetrahydropyranyl, benzoimidazolyl, benzopyridyl, pyrrolopyridyl, benzodihydrofuryl, pyrrolyl, pyrazolyl, thiazolyl, oxazolyl, pyrazinyl, indazolyl, benzothienyl, benzofuryl, benzopyrrolyl, benzoimidazolyl, benzothiazolyl, benzoxazolyl, benzopyridyl, benzopyrimidyl, benzopyrazinyl, piperazinyl, azabicyclo[3.2.1]octanyl, azabicyclo[5.2.0]nonanyl, oxatricyclo[5.3.1.1]dodecyl, azaadamantyl, oxaspiro[3.3]heptanyl,

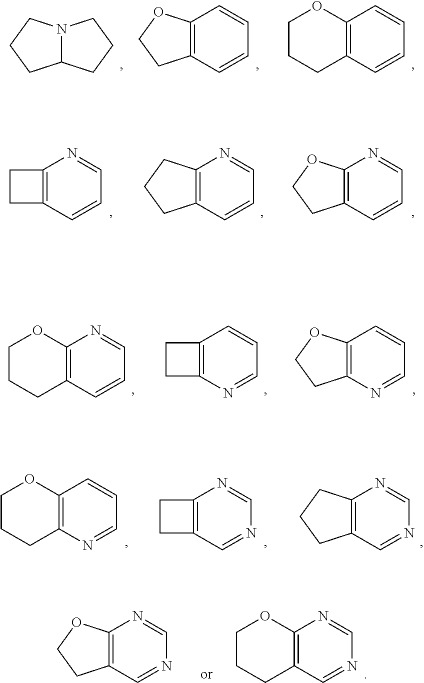

“Heterocyclyl” or “heterocycle” can be monovalent, divalent, trivalent or tetravalent.

“Spiro ring” or “spiro ring group” refers to a polycyclic group that shares one atom (called a spiro atom) between substituted or unsubstituted monocyclic rings. The number of ring atoms in the spiro ring system includes but is not limited to 5 to 20, 6 to 14, 6 to 12, or 6 to 10, wherein one or more rings may contain 0 or more (including but not limited to 1, 2, 3 or 4) double bonds, and can optionally contain 0 to 5 heteroatoms selected from N, O or S(═O)ₙ.

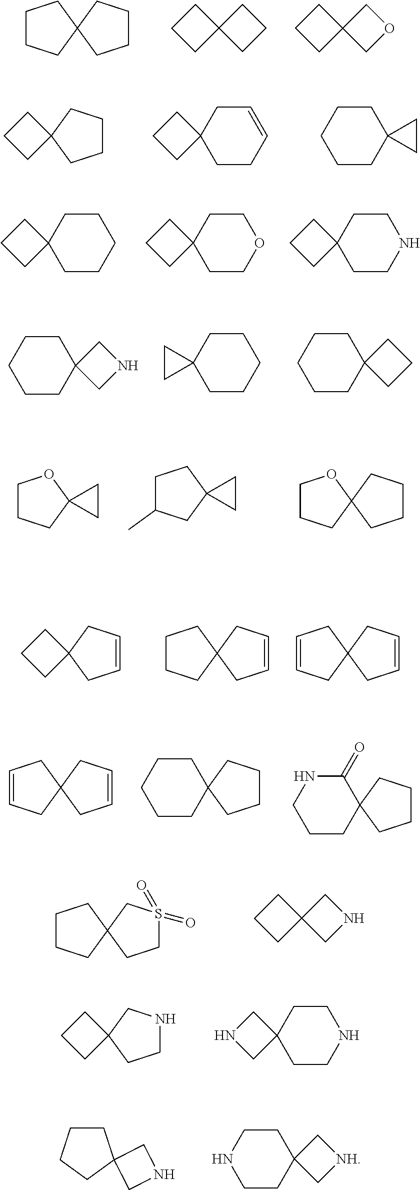

“Spiro ring” or “spiro ring group” can be monovalent, divalent, trivalent or tetravalent.

“Fused ring” or “fused ring group” refers to a polycyclic group in which each ring in the system shares an adjacent pair of atoms with other rings in the system, wherein one or more rings may contain 0 or more (including but not limited to 1, 2, 3 or 4) double bonds, and may be substituted or unsubstituted, and each ring in the fused ring system may contain 0 to 5 heteroatoms or groups containing heteroatoms (including but not limited to N, S(═O)ₙ or O, wherein n is 0, 1 or 2). The number of ring atoms in the fused ring system includes but is not limited to 5 to 20, 5 to 14, 5 to 12, or 5 to 10. Non-limiting examples include:

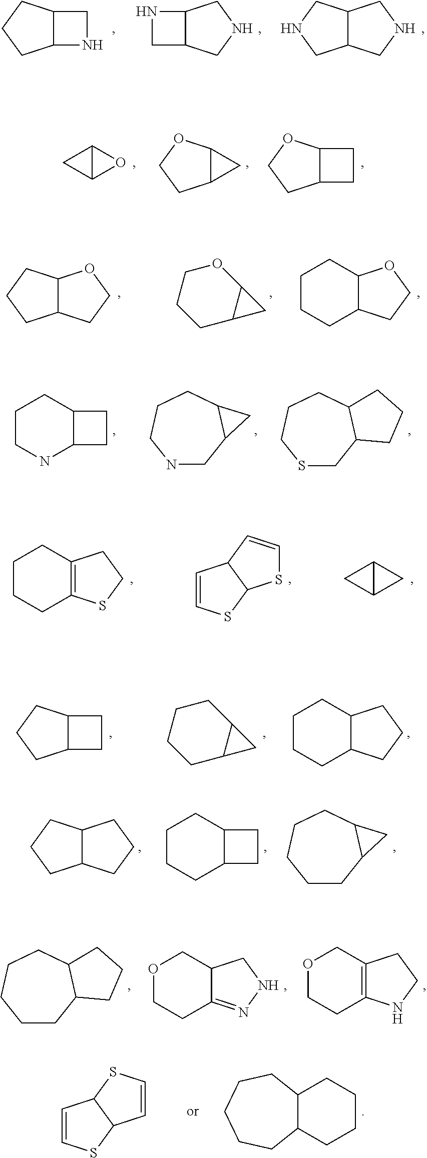

“Fused ring” or “fused ring group” can be monovalent, divalent, trivalent or tetravalent.

“Bridged ring” or “bridged ring group” refers to a substituted or unsubstituted polycyclic group containing any two atoms that are not directly connected, and may contain 0 or more double bonds. Any ring in the fused ring system may contain 0 to 5 groups selected from heteroatoms or groups containing heteroatoms (including but not limited to N, S(═O)ₙ or O, wherein n is 0, 1 or 2). The number of ring atoms includes but is not limited to 5 to 20, 5 to 14, 5 to 12 or 5 to 10. Non-limiting examples include

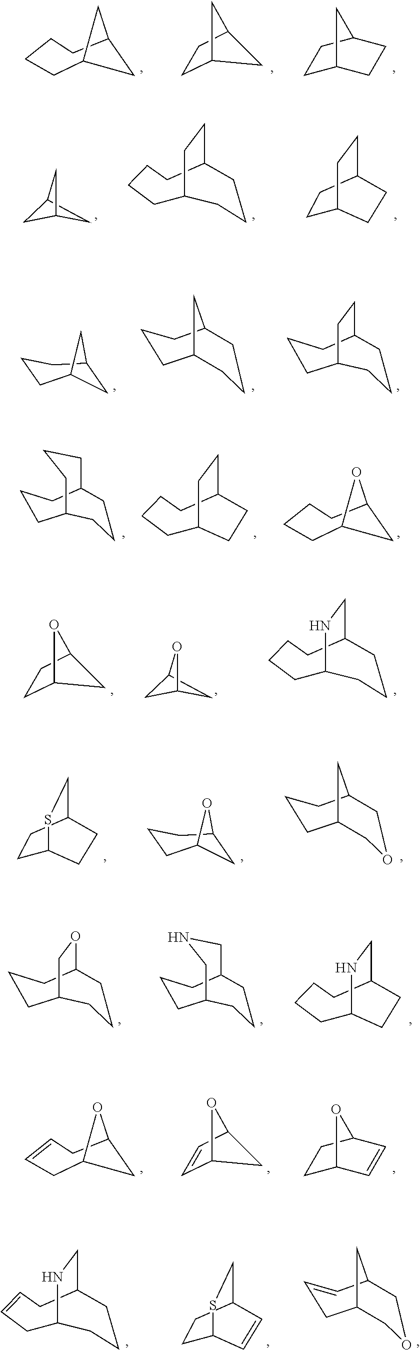

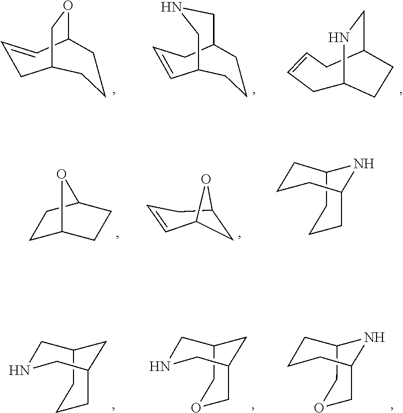

cubane or adamantane. “Bridged ring” or “bridged ring group” can be monovalent, divalent, trivalent or tetravalent.

“Carbospiro ring”, “spiro ring carbocyclyl”, “spirocarbocyclyl” or “carbospiro ring group” refers to a “spiro ring” with a ring system consisting only of carbon atoms. The definition of the “carbospiro ring”, “spiro ring carbocyclyl”, “spirocarbocyclyl” or “carbospiro ring group” herein is consistent with that of a spiro ring.

“Carbo-fused ring”, “fused ring carbocyclyl”, “fused carbocyclyl” or “carbo-fused ring group” refers to a “fused ring” with a ring system consisting only of carbon atoms. The definition of the “carbo-fused ring”, “fused ring carbocyclyl”, “fused carbocyclyl” or “carbo-fused ring group” herein is consistent with that of a fused ring.

“Carbo-bridged ring”, “bridged ring carbocyclyl”, “bridged carbocyclyl” or “carbo-bridged ring group” refers to a “bridged ring” with a ring system consisting only of carbon atoms. The definition of the “carbo-bridged ring”, “bridged ring carbocyclyl”, “bridged carbocyclyl” or “carbo-bridged ring group” herein is consistent with that of a bridged ring.

“Mono-heterocyclic ring”, “monocyclic heterocyclyl” or “mono-heterocyclic ring group” refers to “heterocyclyl” or “heterocycle” with a monocyclic system. The definition of the “heterocyclyl”, “monocyclic heterocyclyl” or “mono-heterocyclic ring group” herein is consistent with that of heterocycle.

“Fused-heterocyclic ring”, “fused-heterocyclic ring group”, “fused ring heterocyclyl” or “fused-heterocyclic ring group” refers to a “fused ring” containing a heteroatom. The definition of the “fused-heterocyclic ring”, “fused-heterocyclic ring group”, “fused ring heterocyclyl” or “fused-heterocyclic ring group” herein is consistent with that of a fused ring.

“Spiro-heterocyclic ring”, “spiro-heterocyclic ring group”, “spiro ring heterocyclyl” or “spiro-heterocyclic ring group” refers to a “spiro ring” containing a heteroatom. The definition of the “spiro-heterocyclic ring”, “spiro-heterocyclic ring group”, “spiro ring heterocyclyl” or “spiro-heterocyclic ring group” herein is consistent with that of a spiro ring.

“Bridged-heterocyclic ring”, “bridged-heterocyclic ring group”, “bridged ring heterocyclyl” or “bridged-heterocyclic ring group” refers to a “bridged ring” containing a heteroatom. The definition of the “bridged-heterocyclic ring”, “bridged-heterocyclic ring group”, “bridged ring heterocyclyl” or “bridged-heterocyclic ring group” herein is consistent with that of a bridged ring.

“Aryl” or “aromatic ring” refers to a substituted or unsubstituted aromatic hydrocarbyl group with a monocyclic ring or a fused ring, wherein the number of ring atoms in the aromatic ring includes but is not limited to 6 to 18, 6 to 12 or 6 to 10 carbon atoms. The aryl ring can be fused to a saturated or unsaturated carbocycle or heterocycle, wherein the ring connected to the parent structure is an aryl ring. Non-limiting examples include a benzene ring, a naphthalene ring, or

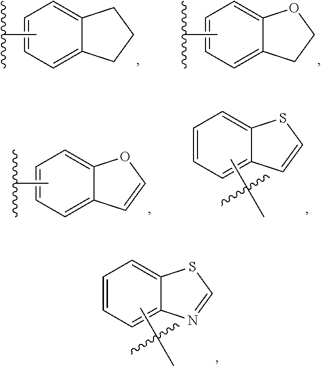

and “aryl” or “aromatic ring” can be monovalent, divalent, trivalent or tetravalent. When divalent, trivalent or tetravalent, the point of connection is on the aryl ring.

“Heteroaryl” or “heteroaromatic ring” refers to a substituted or unsubstituted aromatic hydrocarbyl group containing 1 to 5 heteroatoms or groups containing heteroatoms (including but not limited to N, O or S(═O)n, wherein n is 0, 1 or 2), wherein the number of ring atoms in the heteroaromatic ring includes but is not limited to 5-15, 5-10 or 5-6. Non-limiting examples of heteroaryl include, but are not limited to pyridyl, furyl, thienyl, pyridyl, pyranyl, N-alkylpyrrolyl, pyrimidyl, pyrazinyl, pyridazinyl, imidazolyl, benzopyrazole, benzoimidazole, benzopyridine, pyrrolopyridine, etc. The heteroaryl ring may be fused to a saturated or unsaturated carbocycle or heterocycle, wherein the ring connected to the parent structure is a heteroaryl ring. Non-limiting examples include

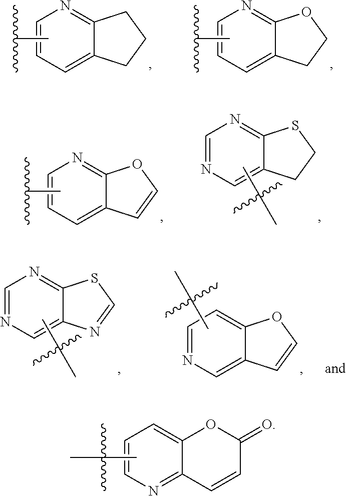

The definition of the “heteroaryl” herein is consistent with this definition. Heteroaryl can be monovalent, divalent, trivalent or tetravalent. When divalent, trivalent or tetravalent, the point of connection is on the heteroaryl ring.

“5-membered ring fused 5-membered heteroaromatic ring” refers to a 5 fused 5-membered fused heteroaromatic ring, wherein at least one of the two fused rings contains at least one heteroatom (including but not limited to O, S or N), and the entire group is aromatic. Non-limiting examples include a pyrrolopyrrole ring, a pyrazolopyrrole ring, a pyrazolopyrazole ring, a pyrrolofuran ring, a pyrazolofuran ring, a pyrrolothiophene ring and a pyrazolothiophene ring.

“5 fused 6-membered heteroaromatic ring” refers to a 5 fused 6-membered fused heteroaromatic ring, wherein at least one of the two fused rings contains at least one heteroatom (including but not limited to O, S or N), and the entire group is aromatic. Non-limiting examples include a benzo 5-membered heteroaryl and 6-membered heteroaromatic ring fused 5-membered heteroaromatic ring.

“Substitution” or “substituted” refers to a substitution with 1 or more (including, but not limited to 2, 3, 4 or 5) substituents including, but not limited to H, F, Cl, Br, I, alkyl, cycloalkyl, alkoxy, haloalkyl, mercaptan, hydroxyl, nitro, mercapto, amino, cyano, isocyano, aryl, heteroaryl, heterocyclyl, bridged ring group, spiro ring group, fused ring group, hydroxyalkyl, ═O, carbonyl, aldehyde, carboxylic acid, carboxylate, —(CH₂)ₘ—C(═O)—R^{a}, —O—(CH₂)ₘ—C(═O)—R^{a}, —(CH₂)ₘC(═O)—NR^{b}R^{c}, —(CH₂)ₘS(═O)ₙR^{a}, —(CH₂)ₘ-alkenyl-R^{a}, OR^{d} or —(CH₂)ₘ-alkynyl-R^{a} (wherein m and n are 0, 1 or 2), arylthio, thiocarbonyl, silyl, —NR^{b}R^{c}, etc., wherein R^{b} and R^{c} are independently selected from H, hydroxyl, amino, carbonyl, alkyl, alkoxy, cycloalkyl, heterocyclyl, aryl, heteroaryl, sulfonyl, or trifluoromethylsulfonyl. Alternatively, R^{b} and R^{c} can form a five- or six-membered cycloalkyl or heterocyclyl.

“Containing 1 to 5 heteroatoms selected from O, S or N” means containing 1, 2, 3, 4 or 5 heteroatoms selected from O, S or N.

“Substituted with 0 to X substituents selected from . . . ” means substituted with 0, 1, 2, 3 . . . X substituents selected from . . . , wherein X is selected from any integer between 1 and 10. For example, “substituted with 0 to 4 substituents selected from . . . ” means substituted with 0, 1, 2, 3 or 4 substituents selected from . . . For example, “substituted with 0 to 5 substituents selected from . . . ” means substituted with 0, 1, 2, 3, 4 or 5 substituents selected from . . . For example, “bridged-heterocyclic ring is optionally further substituted with 0 to 4 substituents selected from H or F” means that the bridged-heterocyclic ring is optionally further substituted with 0, 1, 2, 3 or 4 substituents selected from H or F.

An X- to Y-membered ring (X is selected from an integer less than Y and greater than or equal to 3, and Y is selected from any integer between 4 and 12) includes X+1-, X+2-, X+3-, X+4-, . . . , Y-membered rings. Rings include heterocycle, carbocycle, an aromatic ring, aryl, heteroaryl, cycloalkyl, a mono-heterocyclic ring, a fused-heterocyclic ring, a spiro-heterocyclic ring or a bridged-heterocyclic ring. For example, a “4- to 7-membered mono-heterocyclic ring” refers to a 4-, 5-, 6- or 7-membered mono-heterocyclic ring, and a “5- to 10-membered fused-heterocyclic ring” refers to a 5-, 6-, 7-, 8-, 9- or 10-membered fused-heterocyclic ring.

The term “optional” or “optionally” refers to that the events or circumstances subsequently described may but not necessarily occur, and the description includes the occasions where the events or circumstances occur or do not occur. For example, “alkyl optionally substituted with F” means that the alkyl may but not necessarily be substituted with F, and the description includes the case where the alkyl is substituted with F and the case where the alkyl is not substituted with F.

“Pharmaceutically acceptable salt” or “pharmaceutically acceptable salt thereof” refers to a salt of the compound according to the present invention, which salt maintains the biological effectiveness and characteristics of a free acid or a free base, and is obtained by reacting the free acid with a non-toxic inorganic base or organic base, or reacting the free base with a non-toxic inorganic acid or organic acid.

“Pharmaceutical composition” refers to a mixture of one or more compounds of the present invention, or stereoisomers, tautomers, deuterated compounds, solvates, prodrugs, metabolites, pharmaceutically acceptable salts or co-crystals thereof and other chemical components, wherein “other chemical components” refer to pharmaceutically acceptable carriers, excipients and/or one or more other therapeutic agents.

The term “preparation specification” refers to the weight of the active drug contained in each vial, tablet or other unit preparation.

“Carrier” refers to a material that does not cause significant irritation to an organism and does not eliminate the biological activity and characteristics of a compound administered.

“Animal” is meant to include mammals, such as humans, companion animals, zoo animals, and domestic animals, preferably humans, horses, or dogs.

The term “stereoisomer” refers to an isomer produced as a result of different spatial arrangement of atoms in molecules, including cis-trans isomers, enantiomers and conformational isomers.

“Tautomer” refers to a functional group isomer produced by the rapid movement of an atom in two positions in a molecule, such as keto-enol isomerization and amide-imino alcohol isomerization.

“IC₅₀” refers to the concentration of a medicament or inhibitor required to inhibit half of a given biological process (or a component of the process such as an enzyme, a receptor and a cell).

### BRIEF DESCRIPTION OF THE DRAWINGS

FIG. 1 shows the results of growth inhibition of compound 6 on MOLT-4 xenograft tumor models in nude mice (****P<0.0001 versus the vehicle control group, two-way ANOVA and then Dunnnett's test).

### DETAILED DESCRIPTION OF EMBODIMENTS

The technical solutions of the present invention will be described in detail below in conjunction with examples, but the protection scope of the present invention includes but is not limited thereto.

To achieve the objectives of the present invention, according to organic synthesis techniques known to those skilled in the art, and starting from commercially available chemicals and/or compounds described in chemical documents, the prepared compounds, “commercially available chemicals”, for use in the reactions described herein are obtained from standard commercial sources, including Shanghai Aladdin Bio-Chem Technology Co., Ltd., Shanghai Macklin Biochemical Co., Ltd., Sigma-Aldrich, Alfa Aesar (China) Chemical Co., Ltd., Tokyo Chemical Industry (Shanghai) Co., Ltd., Energy Chemical Co., Ltd., Shanghai Titan Scientific Co., Ltd., Kelong Chemical Co., Ltd., J&K Scientific and the like.

References and monographs in the art introduce in detail the synthesis of reactants that can be used to prepare the compounds described herein, or provide articles describing the preparation method for reference. The references and monographs include: “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992; Fuhrhop, J. and Penzlin G. “Organic Synthesis: Concepts, Methods, Starting Materials”, Second, Revised and Enlarged Edition (1994) John Wiley & Sons ISBN: 3 527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups”, John Wiley & Sons, in 73 volumes.

Specific and similar reactants can be selectively identified by the indexes of known chemicals prepared by the Chemical Abstracts Service of the American Chemical Society, wherein the indexes are available in most public libraries or university libraries and online. Chemicals that are known but not commercially available in the catalog are optionally prepared by custom chemical synthesis plants, wherein many of standard chemical supply plants (such as those listed above) provide custom synthesis services. Reference document for the preparation and selection of the pharmaceutically acceptable salts of the compounds described herein is P. H. Stahl & C. G. Wermuth “Handbook of Pharmaceutical Salts”, Verlag Helvetica Chimica Acta, Zurich, 2002.

The technical solutions of the present invention will be described in detail by the following examples, but the scope of protection of the present invention includes but is not limited thereto.

The compounds used in the reactions described herein are prepared according to organic synthesis techniques known to those skilled in the art, and starting from commercially available chemicals and(or) compounds described in chemical documents. “Commercially available chemicals” are obtained from regular commercial sources, and suppliers include: Titan Technology Co., Ltd., Energy Chemical Co., Ltd., Shanghai Demo Co., Ltd., Chengdu Kelong Chemical Co., Ltd., Accela ChemBio Co., Ltd., PharmaBlock Sciences (Nanjing), Inc., WuXi Apptec Co., Ltd., J&K Scientific Co., Ltd., etc.

The structures of the compounds are determined by nuclear magnetic resonance (NMR) or (and) mass spectrometry (MS). The NMR shift (δ) is given in the unit of 10⁻⁶ (ppm). NMR is determined with (Bruker Avance III 400 and Bruker Avance 300) nuclear magnetic resonance instrument; the solvents for determination are deuterated dimethyl sulfoxide (DMSO-d₆), deuterated chloroform (CDCl₃) and deuterated methanol (CD₃OD); and the internal standard is tetramethylsilane (TMS).

MS is determined with Agilent 6120B (ESI) and Agilent 6120B (APCI);

HPLC is determined with Agilent 1260DAD high pressure liquid chromatograph (Zorbax SB-C18 100×4.6 mm, 3.5 μM);

Yantai Huanghai HSGF254 or Qingdao GF254 silica gel plate is used as a thin layer chromatography silica plate, and the silica gel plate for the thin layer chromatography (TLC) is of the specification of 0.15 mm-0.20 mm, and the specification when separating and purifying a product by thin layer chromatography is 0.4 mm-0.5 mm;
and for the column chromatography, Yantai Huanghai silica gel of 200-300 mesh silica gel is generally used as a carrier.
SEM:

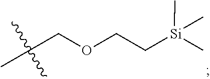 [SMILES: *COCC[Si](C)(C)C]

### THP:

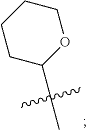 [SMILES: *C1CCCCO1]

Boc: tert-butoxycarbonyl; Ms:

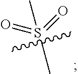

### TBS:

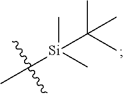 [SMILES: *[Si](C)(C)C(C)(C)C]

MTBE: methyl tert-butyl ether; Bn:

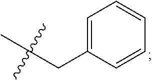 [SMILES: *Cc1ccccc1]

DIPEA: N,N-diisopropylethylamine; DMAc: N,N-dimethylacetamide; DMSO: dimethyl sulfoxide; DCM: dichloromethane; Cbz:

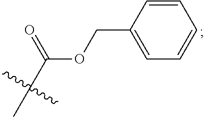 [SMILES: *C(=O)OCc1ccccc1]

NMP: N-methylpyrrolidone; Troc: 2,2,2-trichloroethoxycarbonyl; DMAP: 4-dimethylaminopyridine;
EDCI: CAS 25952-53-8; HOBT: CAS: 2592-95-2; HATU: 148893-10-1;
intermediate 1: 7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptanoic acid (see WO 2019144117 for the synthetic method);
intermediate 2: 2,2,2-trichloroethyl (R)-4-(4-(phenylthio)-3-((4-aminosulfonyl-2-((trifluoromethyl)sulfonyl)phenyl)amino)butyl)piperazine-1-carboxylate (see WO 2017184995 for the synthetic method);
intermediate 3: 8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxocaprylic acid (see WO 2020163823 of the synthetic method).

### Example 1

(2S,4R)-1-((2S)-2-(7-(3-((R)-3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)-3,8-diazabicyclo[3.2.1]octan-8-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 1)

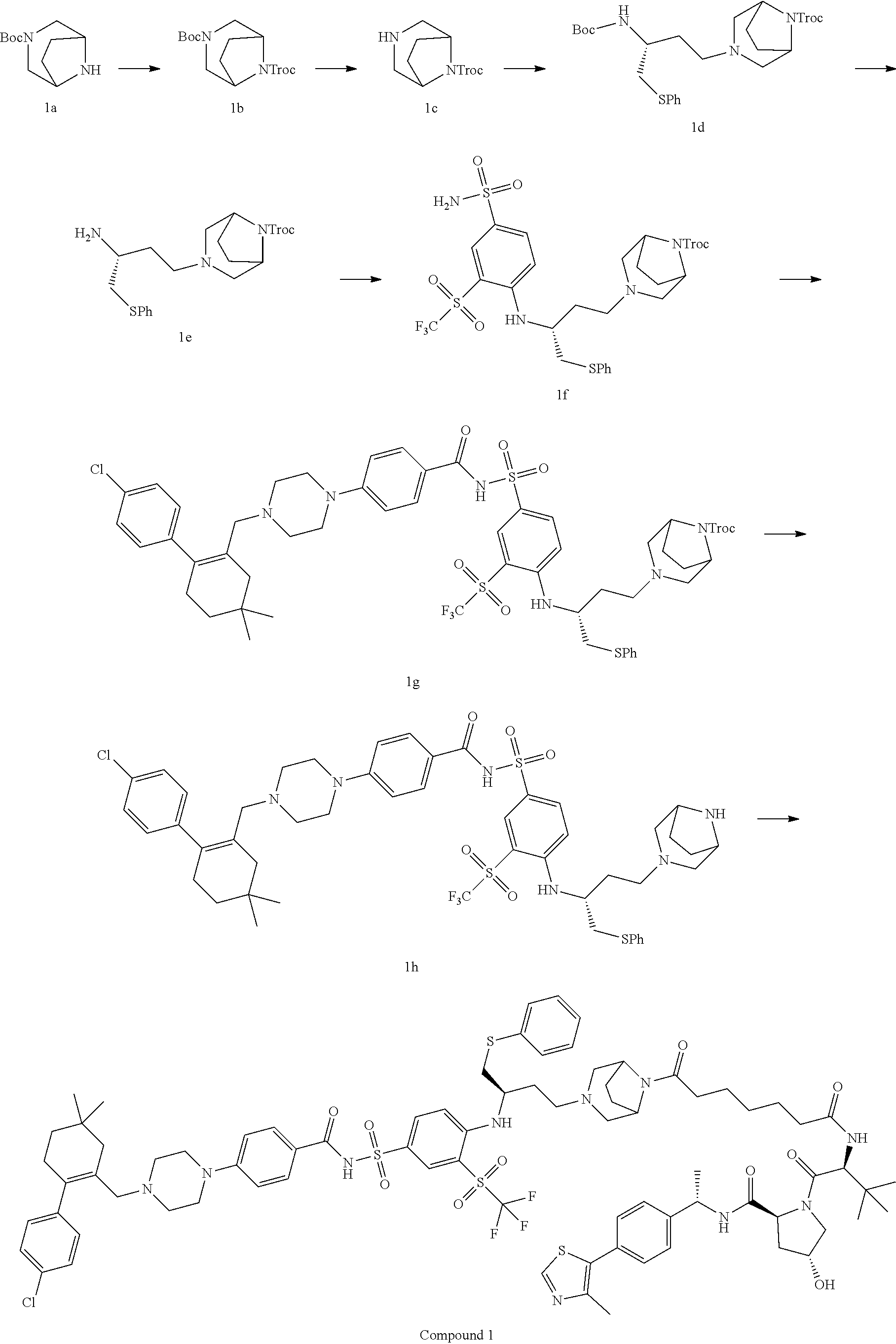

### Step 1: Preparation of 1b

1a (0.85 g, 4.0 mmol) was dissolved in 20 mL of dichloromethane and triethylamine (0.51 g, 5.0 mmol) was added. The mixture was cooled to 0° C. and 2,2,2-trichloroethyl chloroformate (1.0 g, 4.7 mmol) was slowly added dropwise. Then the mixture was slowly warmed to room temperature and reacted for 2 h. After the reaction was completed, 50 mL of dichloromethane and 50 mL of water were added. The organic phase was separated, dried over anhydrous sodium sulfate and concentrated under reduced pressure to afford crude 1b (1.5 g).

### Step 2: Preparation of 1b

Crude 1b (1.5 g) was added to 50 mL of 2 mol/L ethyl acetate hydrogen chloride solution and the mixture was reacted at room temperature for 3 h. The reaction system was filtered and 50 mL of dichloromethane was added to the filter cake. Saturated sodium bicarbonate solution (50 mL) was added and the mixture was stirred until the solid was completely dissolved. The organic phase was separated, dried over anhydrous sodium sulfate and concentrated under reduced pressure to afford crude 1b (0.9 g).

LCMS m/z=287.0 [M+1]⁺.

### Step 3: Preparation of 1d

The above-mentioned crude 1c (0.9 g) was added to 50 mL of 1,2-dichloroethane. Tert-butyl (R)-(4-oxo-1-(phenylthio)butan-2-yl)carbamate (see Nature Communications, 2020, 11, 1996 for the synthetic method) (0.95 g, 3.2 mmol) was added and the mixture was reacted at room temperature for 1 h. Sodium triacetoxyborohydride (0.72 g, 3.4 mmol) was added and the resulting mixture was reacted at room temperature for 12 h. Dichloromethane (50 mL) was added to dilute the reaction solution. Saturated sodium bicarbonate solution was used to adjust the reaction mixture to pH 9.0. The organic phase was separated, dried over anhydrous sodium sulfate and concentrated under reduced pressure. The resulting crude was separated and purified by chromatographic column on silica gel (petroleum ether:ethyl acetate (v/v)=1:1) to afford 1d (1.6 g, total yield over three steps from compound 1a: 71%).

LCMS m/z=566.1 [M+1]⁺.

### Step 4: Preparation of 1e

1d (1.6 g, 2.83 mmol) was added to 50 mL of 2 mol/L ethyl acetate hydrogen chloride solution and the mixture was reacted at room temperature for 3 h. The reaction system was filtered and 50 mL of dichloromethane was added to the filter cake. Saturated sodium bicarbonate solution (50 mL) was added and the mixture was stirred until the solid was completely dissolved. The organic phase was separated, dried over anhydrous sodium sulfate and concentrated under reduced pressure to afford crude 1e (1.3 g).

LCMS m/z=466.0 [M+1]⁺.

### Step 5: Preparation of 1f

The above-mentioned crude 1e (1.3 g) was added to 30 mL of acetonitrile. Triethylamine (4.0 g, 4.0 mmol) and 4-fluoro-3-((trifluoromethyl)sulfonyl) benzenesulfonamide (1.0 g, 3.3 mmol) were added and the mixture was refluxed and reacted at 85° C. for 3 h. The reaction mixture was cooled to room temperature. The reaction solution was concentrated under reduced pressure and the resulting crude was separated and purified by chromatographic column on silica gel (petroleum ether:ethyl acetate (v/v)=2:1) to afford 1f (1.8 g, yield over two steps from compound 1d: 84%).

LCMS m/z=753.0 [M+1]⁺.

### Step 6: Preparation of 1g

1f (1.8 g, 2.4 mmol) was added to 100 mL of dichloromethane. 4-(4-((4′-chloro-4,4-dimethyl-3,4,5,6-tetrahydro-[1,1′-biphenyl]-2-yl)methyl)piperazin-1-yl)benzoic acid (see WO 2017101851 for the synthetic method) (1.31 g, 3.0 mmol), DMAP (0.6 g, 4.9 mmol) and EDCI (0.96 g, 5.0 mmol) were sequentially added and the mixture was reacted at room temperature for 12 h. To the reaction system was added 100 mL of water. The organic phase was separated, dried over anhydrous sodium sulfate and concentrated under reduced pressure and the resulting crude was separated and purified by chromatographic column on silica gel (ethyl acetate) to afford 1g (2.0 g, yield: 71.0%).

LCMS m/z=588.1 [M/2+1]⁺.

### Step 7: Preparation of 1h

1g (2.0 g, 1.7 mmol) was added to 40 mL of tetrahydrofuran. Zinc powder (6.5 g, 100.0 mmol) and 1.2 mL of acetic acid were sequentially added and the mixture was reacted at room temperature for 5 h. The reaction system was filtered and the filtrate was concentrated under reduced pressure. The residue was then diluted with 100 mL of dichloromethane and then 50 mL of saturated sodium bicarbonate solution was added. The organic phase was separated, dried over anhydrous sodium sulfate and concentrated under reduced pressure to afford crude 1h (1.5 g).

LCMS m/z=500.3 [M/2+1]⁺.

### Step 8: Preparation of Compound 1

The above-mentioned crude 1h (1.5 g) was added to 30 mL of DMF. Intermediate 1 (0.96 g, 1.6 mmol), triethylamine (0.6 g, 4.9 mmol) and HATU (0.78 g, 2.1 mmol) were sequentially added and the mixture was reacted at room temperature for 12 h. To the reaction system was added water (200 mL), and a solid was precipitated. The system was filtered and the filter cake was passed through Pre-HPLC (instrument and preparative column: using Glison GX-281 preparative liquid phase chromatographic instrument, preparative column model: Sunfire C18, 5 μm, inner diameter×length=30 mm×150 mm). Preparation method: the crude was dissolved with methanol and dimethyl sulfoxide, and filtered with a 0.45 m filter membrane, to prepare into a sample liquid. Mobile phase system: acetonitrile/water (containing 0.1% TFA). Gradient elution method: gradient elution with acetonitrile from 5% to 60% (elution time: 15 min). To the preparative solution were added 100 mL of dichloromethane and 60 mL of saturated sodium bicarbonate solution and the mixture was stirred for 1 h. The organic phase was separated, dried over anhydrous sodium sulfate and concentrated under reduced pressure to afford compound 1 (0.7 g, yield over two steps from compound 1g: 26%).

¹H NMR (400 MHz, CDCl₃) δ 8.59 (s, 1H), 8.28 (s, 1H), 8.04 (d, 1H), 7.69-7.59 (m, 2H), 7.44-7.08 (m, 12H), 6.96-6.87 (m, 2H), 6.85-6.76 (m, 1H), 6.68 (d, 2H), 6.61-6.49 (m, 1H), 6.32-6.17 (m, 1H), 5.07-4.95 (m, 1H), 4.70-4.58 (m, 1H), 4.56-4.36 (m, 3H), 4.08-3.70 (m, 3H), 3.56-3.45 (m, 1H), 3.28-3.12 (m, 4H), 3.10-3.00 (m, 1H), 2.98-2.85 (m, 1H), 2.84-2.74 (m, 2H), 2.68-2.49 (m, 2H), 2.48-1.86 (m, 23H), 1.78-1.15 (m, 15H), 1.05-0.80 (m, 15H).

LCMS m/z=523.5 [M/3+1]⁺.

Example 2

(2S,4R)-1-((2S)-2-(7-(5-((R)-3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)-2,5-diazabicyclo[2.2.1]heptan-2-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 2)

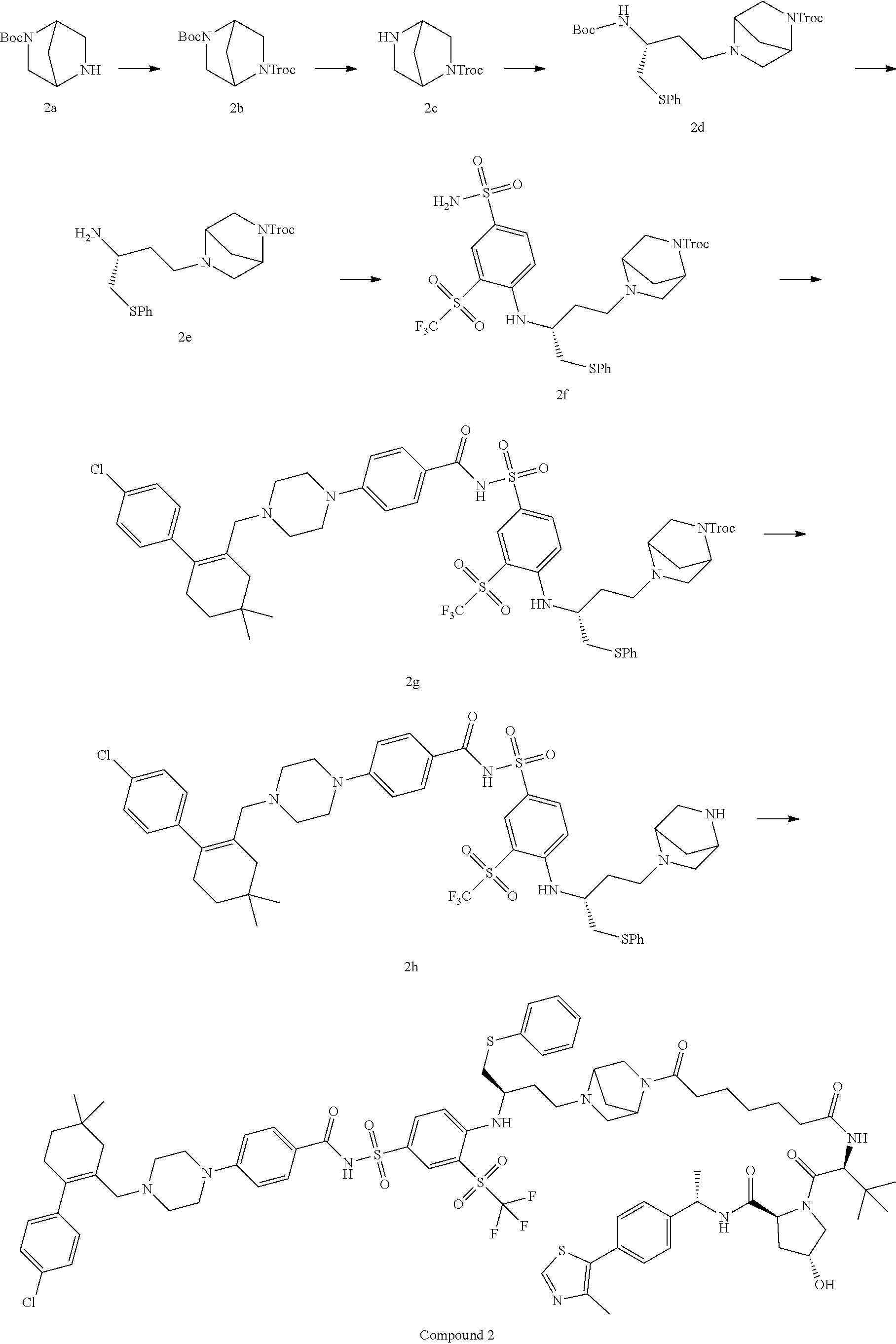

### Step 1: Preparation of 2b

2a (0.8 g, 4.0 mmol) was dissolved in 20 mL of dichloromethane and triethylamine (0.51 g, 5.0 mmol) was added. The mixture was cooled to 0° C. and 2,2,2-trichloroethyl chloroformate (1.0 g, 4.7 mmol) was slowly added dropwise. Then the mixture was slowly warmed to room temperature and reacted for 2 h. After the reaction was completed, the reaction solution was diluted with dichloromethane (50 mL) and then 50 mL of water was added. The organic phase was separated, dried over anhydrous sodium sulfate and concentrated under reduced pressure to afford crude 2b (1.4 g).

### Step 2: Preparation of 2c

The above-mentioned crude 2b (1.4 g) was added to 50 mL of 2 mol/L ethyl acetate hydrogen chloride solution and the mixture was reacted at room temperature for 3 h. The reaction system was filtered and 50 mL of DCM was added to the filter cake. Saturated sodium bicarbonate solution (50 mL) was added and the mixture was stirred until the solid was completely dissolved. The organic phase was separated, dried over anhydrous sodium sulfate and concentrated under reduced pressure to afford crude 2c (0.85 g).

LCMS m/z=273.0 [M+1]⁺.

### Step 3: Preparation of 2d

The above-mentioned crude 2c (0.85 g) was added to 50 mL of 1,2-dichloroethane. Tert-butyl (R)-(4-oxo-1-(phenylthio)butan-2-yl)carbamate (0.95 g, 3.2 mmol) was added and the mixture was reacted at room temperature for 1 h. Sodium triacetoxyborohydride (0.72 g, 3.4 mmol) was added and the resulting mixture was reacted at room temperature for 12 h. DCM (50 mL) was added to dilute the reaction solution. Saturated sodium bicarbonate solution was used to adjust the reaction mixture to pH 9.0. The organic phase was separated, dried over anhydrous sodium sulfate and concentrated under reduced pressure. The resulting crude was separated and purified by chromatographic column on silica gel (petroleum ether:ethyl acetate (v/v)=1:1) to afford 2d (1.3 g, total yield over three steps from compound 2a: 59%).

LCMS m/z=552.2 [M+1]⁺.

### Step 4: Preparation of 2e

2d (1.3 g, 2.36 mmol) was added to 50 mL of 2 mol/L ethyl acetate hydrogen chloride solution and the mixture was reacted at room temperature for 3 h. The reaction system was filtered and 50 mL of DCM was added to the filter cake. Saturated sodium bicarbonate solution (50 mL) was added and the mixture was stirred until the solid was completely dissolved. The organic phase was separated, dried over anhydrous sodium sulfate and concentrated under reduced pressure to afford crude 2e (1.0 g).

LCMS m/z=452.0 [M+1]⁺.

### Step 5: Preparation of 2f

The above-mentioned crude 2e (1.0 g) was added to 30 mL of acetonitrile. Triethylamine (4.0 g, 4.0 mmol) and 4-fluoro-3-((trifluoromethyl)sulfonyl) benzenesulfonamide (0.76 g, 2.6 mmol) were added and the mixture was refluxed and reacted at 85° C. for 3 h. The reaction mixture was cooled to room temperature. The reaction solution was concentrated under reduced pressure and the resulting crude was separated and purified by chromatographic column on silica gel (petroleum ether:ethyl acetate (v/v)=2:1) to afford 2f (1.5 g, yield over two steps from compound 2d: 86%).

LCMS m/z=739.0 [M+1]⁺.

### Step 6: Preparation of 2g

2f (1.5 g, 2.0 mmol) was added to 100 mL of dichloromethane. 4-(4-((4′-chloro-4,4-dimethyl-3,4,5,6-tetrahydro-[1,1′-biphenyl]-2-yl)methyl)piperazin-1-yl)benzoic acid (0.87 g, 2.0 mmol), DMAP (0.5 g, 4.1 mmol) and EDCI (0.76 g, 4.0 mmol) were sequentially added and the mixture was reacted at room temperature for 12 h. To the reaction system was added 100 mL of water. The organic phase was separated, dried over anhydrous sodium sulfate and concentrated under reduced pressure and the resulting crude was separated and purified by chromatographic column on silica gel (ethyl acetate) to afford 2g (1.6 g, yield: 69%).

LCMS m/z=581.2 [M/2+1]⁺.

### Step 7: Preparation of 2h

2g (1.6 g, 1.38 mmol) was added to 40 mL of tetrahydrofuran. Zinc powder (6.5 g, 100.0 mmol) and 1.2 mL of acetic acid were sequentially added and the mixture was reacted at room temperature for 5 h. The reaction system was filtered and the filtrate was concentrated under reduced pressure. The residue was then diluted with 100 mL of DCM and then 50 mL of saturated sodium bicarbonate solution was added. The organic phase was separated, dried over anhydrous sodium sulfate and concentrated under reduced pressure to afford crude 2h (1.1 g).

LCMS m/z=493.2 [M/2+1]⁺.

### Step 8: Preparation of Compound 2

The above-mentioned crude 2h (1.1 g) was added to 30 mL of DMF. Intermediate 1 (0.96 g, 1.6 mmol), triethylamine (0.6 g, 4.9 mmol) and HATU (0.78 g, 2.1 mmol) were sequentially added and the mixture was reacted at room temperature for 12 h. To the reaction system was added water (200 mL), and a solid was precipitated. The system was filtered and the filter cake was passed through Pre-HPLC (instrument and preparative column: using Glison GX-281 preparative liquid phase chromatographic instrument, preparative column model: Sunfire C18, 5 μm, inner diameter×length=30 mm×150 mm). Preparation method: the crude was dissolved with methanol and dimethyl sulfoxide, and filtered with a 0.45 m filter membrane, to prepare into a sample liquid. Mobile phase system: acetonitrile/water (containing 0.1% TFA). Gradient elution method: gradient elution with acetonitrile from 5% to 60% (elution time: 15 min). To the preparative solution were added 100 mL of dichloromethane and 60 mL of saturated sodium bicarbonate solution and the mixture was stirred for 1 h. The organic phase was separated, dried over anhydrous sodium sulfate and concentrated under reduced pressure to afford compound 2 (0.05 g, yield over two steps from compound 2g: 2%).

¹H NMR (400 MHz, CDCl₃) δ 8.66 (s, 1H), 8.46-8.34 (m, 1H), 8.12-7.92 (m, 1H), 7.83-7.63 (m, 2H), 7.44-7.05 (m, 13H), 7.04-6.93 (m, 2H), 6.90-6.65 (m, 3H), 6.48-6.30 (m, 1H), 5.16-4.94 (m, 1H), 4.86-4.42 (m, 4H), 4.15-3.97 (m, 2H), 3.70-2.95 (m, 10H), 2.94-1.95 (m, 24H), 1.62-1.20 (m, 14H), 1.10-0.95 (m, 15H).

LCMS m/z=518.9 [M/3+1]⁺.

Example 3

(2S,4R)-1-((S)-2-(7-(4-((R)-3-((4-(N-(4-(1-((4′-chloro-4,4-dimethyl-3,4,5,6-tetrahydro-[1,1′-biphenyl]-2-yl)methyl)piperidin-4-yl)benzoyl)sulfamoyl)-2-((trifluoromethyl)sulfonyl)phenyl)amino)-4-(phenylthio)butyl)piperazin-1-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 3)

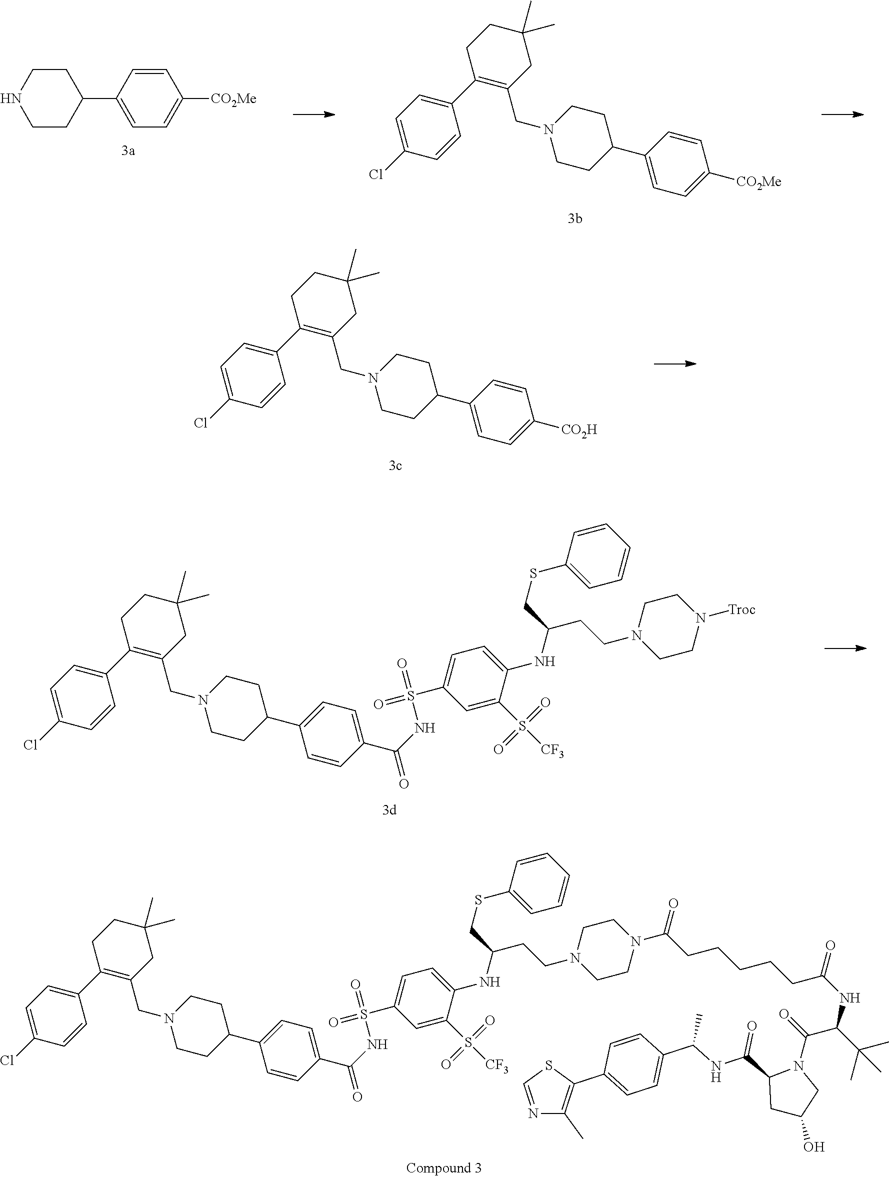

### Step 1: Preparation of 3b

4′-chloro-4,4-dimethyl-3,4,5,6-tetrahydro-[1,1′-biphenyl]-2-carbaldehyde (see WO 2020041406 for the synthetic method) (350 mg, 1.41 mmol) was dissolved in 15 mL of tetrahydrofuran. 3a (see WO 2020212530 for the synthetic method) (309 mg, 1.41 mmol) and 1 mL of tetraisopropyl titanate were sequentially added and the mixture was stirred at room temperature for 4 h. Then sodium triacetoxyborohydride (1.49 g, 7.03 mmol) was added and the resulting mixture was reacted at room temperature for 16 h. To the reaction solution was slowly added 20 mL of saturated sodium bicarbonate solution. The resulting mixture was extracted with ethyl acetate (60 mL×2). The organic phase was washed with 50 mL of saturated sodium chloride solution, dried over anhydrous sodium sulfate and concentrated under reduced pressure and the resulting crude was separated and purified by silica gel column chromatography (ethyl acetate/petroleum ether (v/v)=0:1-1:1) to afford 3b (400 mg, yield: 63%).

LCMS m/z=452.3 [M+1]⁺.

### Step 2: Preparation of 3c

3b (0.40 g, 0.88 mmol) was dissolved in 10 mL of methanol. Water (1 mL) and sodium hydroxide (0.15 g, 3.75 mmol) were added and the mixture was reacted at 80° C. for 10 h. The reaction system was cooled to room temperature and concentrated under reduced pressure. The residue was dissolved in 10 mL of water and washed with 20 mL of methyl tert-butyl ether. The aqueous phase was separated and adjusted to pH 6 with 1 mol/L hydrochloric acid, extracted with ethyl acetate (100 mL×2), dried over anhydrous sodium sulfate and concentrated under reduced pressure to afford crude 3c (0.20 g).

LCMS m/z=438.2 [M+1]⁺.

### Step 3: Preparation of 3d

The above-mentioned crude 3c (200 mg) was dissolved in 15 mL of DCM. Intermediate 2 (330 mg, 0.45 mmol), DMAP (110 mg, 0.9 mmol) and EDCI (180 mg, 0.95 mmol) were sequentially added and the mixture was reacted at room temperature for 16 h. To the reaction system was slowly added 30 mL of water and the mixture was extracted with DCM (60 mL×2). The organic phase was washed with 50 mL of water, dried over anhydrous sodium sulfate and concentrated under reduced pressure and the resulting crude was separated and purified by silica gel column chromatography (dichloromethane/methanol (v/v)=9:1) to afford 3d (260 mg, yield over two steps from compound 3b: 26%).

LCMS m/z=574.5 [M/2+1]⁺.

### Step 4: Preparation of Compound 3

3d (260 mg, 0.23 mmol) was dissolved in 20 mL of tetrahydrofuran. Acetic acid (0.6 mL) and zinc powder (960 mg, 14.8 mmol) were sequentially added and the mixture was reacted at room temperature for 16 h. The reaction system was filtered and to the filtrate was added water (10 mL). The mixture was then extracted with 20 mL of ethyl acetate. The organic phase was separated, dried over anhydrous sodium sulfate and concentrated under reduced pressure to afford a crude (250 mg). The above-mentioned crude (250 mg) was dissolved in 10 mL of DCM. Triethylamine (0.2 mL), intermediate 1 (154 mg, 0.26 mmol) and HATU (142 mg, 0.37 mmol) were sequentially added and the mixture was reacted at room temperature for 1 h. To the reaction system was added 20 mL of water and the mixture was extracted with 20 mL of ethyl acetate. The organic phase was washed with 20 mL of water, dried over anhydrous sodium sulfate and concentrated under reduced pressure and the resulting crude was passed through Pre-HPLC (instrument and preparative column: using Glison GX-281 preparative liquid phase chromatographic instrument, preparative column model: Sunfire C18, 5 μm, inner diameter×length=30 mm×150 mm). Preparation method: the crude was dissolved with methanol and dimethyl sulfoxide, and filtered with a 0.45 m filter membrane, to prepare into a sample liquid. Mobile phase system: acetonitrile/water (containing 0.1% TFA). Gradient elution method: gradient elution with acetonitrile from 5% to 60% (elution time: 15 min). To the preparative solution were added 100 mL of dichloromethane and 60 mL of saturated sodium bicarbonate solution and the mixture was stirred for 1 h. The organic phase was separated, dried over anhydrous sodium sulfate and concentrated under reduced pressure to afford compound 3 (40 mg, yield from compound 3d: 11%).

¹H NMR (400 MHz, CDCl₃) δ 8.66 (s, 1H), 8.32 (s, 1H), 8.05-7.97 (m, 1H), 7.95-7.84 (m, 2H), 7.64-7.55 (m, 1H), 7.48-7.12 (m, 12H), 7.09-6.96 (m, 3H), 6.86-6.78 (m, 1H), 6.65-6.57 (m, 1H), 6.34-6.22 (m, 1H), 5.16-5.03 (m, 1H), 4.71-4.61 (m, 1H), 4.50-4.39 (m, 2H), 4.12-3.96 (m, 1H), 3.96-3.79 (m, 1H), 3.70-3.45 (m, 2H), 3.40-2.88 (m, 9H), 2.58-1.98 (m, 23H), 1.77-1.38 (m, 14H), 1.36-1.18 (m, 2H), 1.08-0.82 (m, 15H).

LCMS m/z=770.8 [M/2+1]⁺.

Example 4

(2S,4R)-1-((S)-2-(7-(4-((R)-3-((4-(N-(4-(4-((4′-cyano-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazin-1-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 4) trifluoroacetate

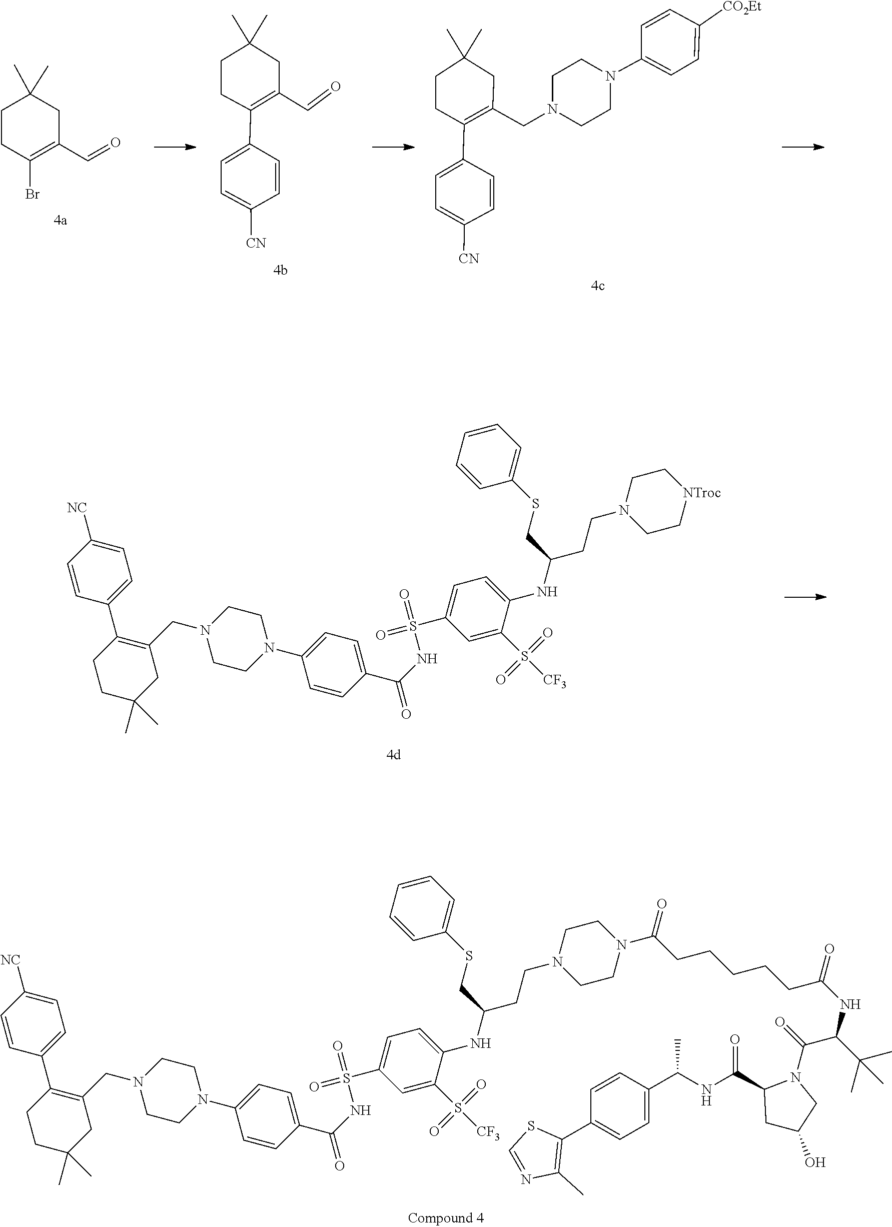

### Step 1: Preparation of 4b

4a (see WO 2020041406 for the synthetic method) (1.00 g, 4.61 mmol) and (4-cyanophenyl)boronic acid (1.01 g, 6.87 mmol) were dissolved in 20 mL of dioxane and 2 mL of water. Potassium acetate (1.36 g, 13.8 mmol) and Pd(dppf)Cl₂ (0.33 g, 0.45 mmol) were sequentially added and the mixture was reacted at 90° C. for 4 h. The reaction solution was cooled to room temperature and to the reaction solution was slowly added 100 mL of water. The mixture was extracted with ethyl acetate (60 mL×2), dried over anhydrous sodium sulfate and concentrated under reduced pressure and the resulting crude was separated and purified by silica gel column chromatography (ethyl acetate/petroleum ether (v/v)=0:1-1:10) to afford 4b (0.95 g, yield: 86%).

LCMS m/z=240.3 [M+1]⁺.

### Step 2: Preparation of 4c

4b (510 mg, 2.13 mmol) was dissolved in 15 mL of tetrahydrofuran. Ethyl 4-(piperazin-1-yl)benzoate (500 mg, 2.13 mmol) was added, followed by 1 mL of tetraisopropyl titanate. The mixture was stirred at room temperature for 4 h and then sodium triacetoxyborohydride (1.35 g, 6.37 mmol) was added. The resulting mixture was reacted at room temperature for 16 h. To the reaction solution was slowly added 20 mL of saturated aqueous sodium bicarbonate solution. The resulting mixture was extracted with 60 mL of ethyl acetate twice and the organic phase was washed with 50 mL of saturated sodium chloride, dried over anhydrous sodium sulfate and concentrated under reduced pressure. The resulting crude was separated and purified by silica gel column chromatography (ethyl acetate/petroleum ether (v/v)=0:1-1:1) to afford 4c (550 mg, yield: 56%).

LCMS m/z=458.3 [M+1]⁺.

### Step 3: Preparation of 4d

4c (0.55 g, 1.20 mmol) was dissolved in 20 mL of methanol. Water (2 mL) was added, followed by sodium hydroxide (0.24 g, 6.0 mmol) and the mixture was stirred at 80° C. for 10 h. The reaction solution was cooled to room temperature and concentrated under reduced pressure. The residue was dissolved with 10 mL of water and then extracted with 20 mL of methyl tert-butyl ether to remove the impurities. The aqueous phase was separated, adjusted to pH 6 with 1 mol/L hydrochloric acid, extracted with ethyl acetate (100 mL×2), dried over anhydrous sodium sulfate and concentrated under reduced pressure to afford a crude (0.35 g). The above-mentioned crude (350 mg) was dissolved in 20 mL of DCM. Intermediate 2 (590 mg, 0.81 mmol), DMAP (200 mg, 1.64 mmol) and EDCI (310 mg, 1.62 mmol) were sequentially added and the mixture was reacted at room temperature for 16 h. To the reaction system was slowly added 30 mL of water and the mixture was extracted with 60 mL of DCM twice. The organic phase was washed with 50 mL of water, dried over anhydrous sodium sulfate and concentrated under reduced pressure and the resulting crude was separated and purified by silica gel column chromatography (dichloromethane/methanol (v/v)=9:1) to afford 4d (310 mg, yield: 23%).

### Step 4: Preparation of Trifluoroacetate of Compound 4

4d (310 mg, 0.27 mmol) was dissolved in 20 mL of tetrahydrofuran. Acetic acid (0.6 mL) and zinc powder (960 mg, 14.8 mmol) were sequentially added and the mixture was reacted at room temperature for 16 h. The reaction system was filtered and to the filtrate was added water (20 mL). The mixture was then extracted with 20 mL of ethyl acetate. The organic phase was separated, dried over anhydrous sodium sulfate and concentrated under reduced pressure to afford a crude (250 mg). The above-mentioned crude (250 mg) was dissolved in 10 mL of DCM. Triethylamine (0.2 mL), intermediate 1 (150 mg, 0.26 mmol) and HATU (142 mg, 0.37 mmol) were sequentially added and the mixture was reacted at room temperature for 1 h. To the reaction system was added 20 mL of water and the mixture was extracted with 20 mL of dichloromethane. The organic phase was washed with 20 mL of water, dried over anhydrous sodium sulfate and concentrated under reduced pressure and the resulting crude was passed through Pre-HPLC (instrument and preparative column: using Glison GX-281 preparative liquid phase chromatographic instrument, preparative column model: Sunfire C18, 5 μm, inner diameter×length=30 mm×150 mm). Preparation method: the crude was dissolved with methanol and dimethyl sulfoxide, and filtered with a 0.45 m filter membrane, to prepare into a sample liquid. Mobile phase system: acetonitrile/water (containing 0.1% TFA). Gradient elution method: gradient elution with acetonitrile from 5% to 60% (elution time: 15 min). Lyophilization was performed to afford the trifluoroacetate of compound 4 (20 mg).

¹H NMR (400 MHz, CDCl₃) δ 8.66 (s, 1H), 8.30 (s, 1H), 8.13-8.00 (m, 1H), 7.90-7.75 (m, 2H), 7.70-7.52 (m, 3H), 7.40-7.28 (m, 6H), 7.26-7.12 (m, 5H), 6.94-6.80 (m, 1H), 6.75-6.55 (m, 3H), 6.35-6.15 (m, 1H), 5.15-4.99 (m, 1H), 4.70-4.58 (m, 1H), 4.47-4.33 (m, 2H), 4.09-3.99 (m, 1H), 3.98-3.82 (m, 1H), 3.68-3.40 (m, 2H), 3.36-2.93 (m, 9H), 2.80-2.66 (m, 2H), 2.50 (s, 3H), 2.40-1.97 (m, 21H), 1.65-1.42 (m, 10H), 1.32-1.20 (m, 2H), 1.10-0.90 (m, 15H).

LCMS m/z=767.4 [M/2+1]⁺.

Example 5

(2S,4R)-1-((S)-2-(7-(4-((R)-3-((4-(N-(4-(4-((4-(4-chlorophenyl)-5,6-dihydro-2H-pyran-3-yl)methyl)piperazin-1-yl)benzoyl)sulfamoyl)-2-((trifluoromethyl)sulfonyl)phenyl)amino)-4-(phenylthio)butyl)piperazin-1-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 5)

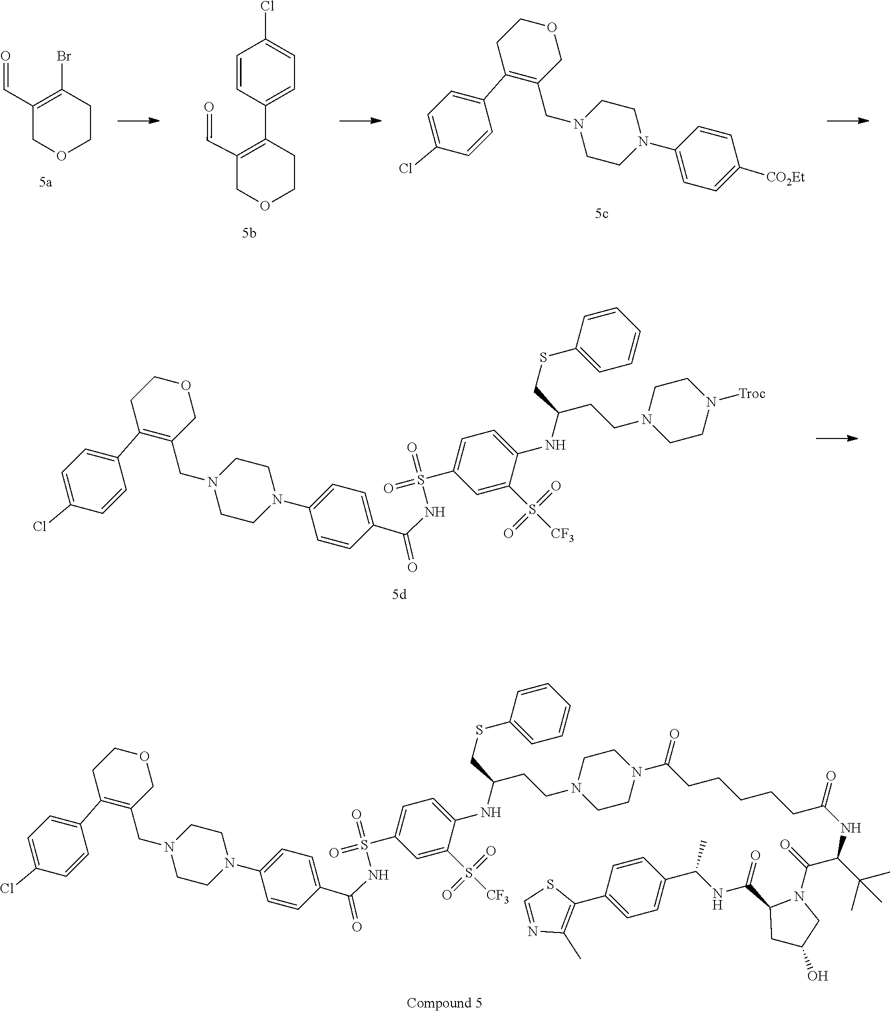

### Step 1: Preparation of 5b

Under nitrogen protection, 5a (see European Journal of Medicinal Chemistry, 2018, 149, 79-89 for the synthetic method) (2 g, 10.47 mmol) and (4-chlorophenyl)boronic acid (2.47 g, 15.80 mmol) were dissolved in 20 mL of dioxane and 2 mL of water. Potassium acetate (3.11 g, 31.69 mmol) and Pd(dppf)Cl₂ (0.2 g, 0.27 mmol) were sequentially added and the mixture was reacted at 90° C. for 4 h. The reaction solution was cooled to room temperature and to the reaction solution was slowly added 100 mL of water. The mixture was extracted with ethyl acetate (60 mL×2), dried over anhydrous sodium sulfate and concentrated under reduced pressure and the resulting crude was separated and purified by silica gel column chromatography (ethyl acetate/petroleum ether (v/v)=0:1-1:10) to afford 5b (1.76 g, yield: 75%).

¹H NMR (400 MHz, CDCl₃) δ 9.51 (s, 1H), 7.46-7.36 (m, 2H), 7.26-7.18 (m, 2H), 4.50-4.40 (m, 2H), 3.92 (t, 2H), 2.67-2.59 (m, 2H).

### Step 2: Preparation of 5c

5b (1.67 g, 7.50 mmol) was dissolved in 15 mL of tetrahydrofuran. Ethyl 4-(piperazin-1-yl)benzoate (1.64 g, 7.00 mmol) and 5 mL of tetraisopropyl titanate were sequentially added and the mixture was stirred at room temperature for 4 h. Then sodium triacetoxyborohydride (3.17 g, 14.96 mmol) was added and the resulting mixture was reacted at room temperature for 16 h. To the reaction solution was slowly added 20 mL of saturated sodium bicarbonate solution. The resulting mixture was extracted with ethyl acetate (60 mL×2). The organic phase was washed with 50 mL of saturated sodium chloride solution, dried over anhydrous sodium sulfate and concentrated under reduced pressure and the resulting crude was separated and purified by silica gel column chromatography (ethyl acetate/petroleum ether (v/v)=0:1-1:1) to afford 5c (2.01 g, yield: 61%).

### Step 3: Preparation of 5d

5c (0.40 g, 0.91 mmol) was dissolved in 10 mL of methanol. Water (1 mL) and sodium hydroxide (0.15 g, 3.75 mmol) were added and the mixture was reacted at 80° C. for 10 h. The reaction solution was cooled to room temperature and concentrated under reduced pressure. The residue was dissolved with 10 mL of water and then extracted with 20 mL of methyl tert-butyl ether to remove the impurities. The aqueous phase was separated, adjusted to pH 6 with 1 mol/L hydrochloric acid, extracted with ethyl acetate (100 mL×2), dried over anhydrous sodium sulfate and concentrated under reduced pressure to afford a crude (0.20 g). The above-mentioned crude (200 mg) was dissolved in 15 mL of DCM. Intermediate 2 (330 mg, 0.45 mmol), DMAP (110 mg, 0.9 mmol) and EDCI (180 mg, 0.94 mmol) were sequentially added and the mixture was reacted at room temperature for 16 h. To the reaction system was slowly added 30 mL of water and the mixture was extracted with DCM (60 mL×2). The organic phase was washed with 50 mL of water, dried over anhydrous sodium sulfate and concentrated under reduced pressure and the resulting crude was separated and purified by silica gel column chromatography (dichloromethane/methanol (v/v)=9:1) to afford 5d (260 mg, yield: 25%).

### Step 4: Preparation of Compound 5

5d (260 mg, 0.23 mmol) was dissolved in 20 mL of tetrahydrofuran. Acetic acid (0.6 mL) and zinc powder (960 mg, 14.8 mmol) were sequentially added and the mixture was reacted at room temperature for 16 h. The reaction system was filtered and to the filtrate was added water (10 mL). The mixture was then extracted with 20 mL of ethyl acetate. The organic phase was separated, dried over anhydrous sodium sulfate and concentrated under reduced pressure to afford a crude (250 mg). The above-mentioned crude (250 mg) was dissolved in 10 mL of DCM. Triethylamine (0.2 mL), intermediate 1 (154 mg, 0.27 mmol) and HATU (142 mg, 0.37 mmol) were sequentially added and the mixture was reacted at room temperature for 1 h. To the reaction system was added 20 mL of water and the mixture was extracted with 20 mL of ethyl acetate. The organic phase was washed with 20 mL of water, dried over anhydrous sodium sulfate and concentrated under reduced pressure and the resulting crude was passed through Pre-HPLC (instrument and preparative column: using Glison GX-281 preparative liquid phase chromatographic instrument, preparative column model: Sunfire C18, 5 μm, inner diameter×length=30 mm×150 mm). Preparation method: the crude was dissolved with methanol and dimethyl sulfoxide, and filtered with a 0.45 m filter membrane, to prepare into a sample liquid. Mobile phase system: acetonitrile/water (containing 0.1% TFA). Gradient elution method: gradient elution with acetonitrile from 5% to 60% (elution time: 15 min). To the preparative solution were added 100 mL of dichloromethane and 60 mL of saturated sodium bicarbonate solution and the mixture was stirred for 1 h. The organic phase was separated, dried over anhydrous sodium sulfate and concentrated under reduced pressure to afford compound 5 (40 mg, yield: 11%).

¹H NMR (400 MHz, CDCl₃) δ 8.68 (s, 1H), 8.35 (d, 1H), 8.09 (dd, 1H), 7.67 (d, 2H), 7.50-7.20 (m, 12H), 7.15-7.01 (m, 3H), 6.83-6.60 (m, 3H), 6.30-6.20 (m, 1H), 5.14-5.02 (m, 1H), 4.78-4.69 (m, 1H), 4.62-4.56 (m, 1H), 4.52-4.45 (m, 1H), 4.33-4.24 (m, 2H), 4.18-4.04 (m, 1H), 4.00-3.80 (m, 3H), 3.76-3.54 (m, 2H), 3.53-3.20 (m, 7H), 3.17-2.90 (m, 4H), 2.60-2.02 (m, 21H), 1.70-1.40 (m, 9H), 1.37-1.15 (m, 2H), 1.07-1.00 (m, 9H).

LCMS m/z=758.5 [M/2+1]⁺.

Example 6

(2S,4R)-1-((S)-2-(7-(4-((R)-3-((4-(N-(4-(4-((2-(4-chlorophenyl)cyclohept-1-en-1-yl)methyl)piperazin-1-yl)benzoyl)sulfamoyl)-2-((trifluoromethyl)sulfonyl)phenyl)amino)-4-(phenylthio)butyl)piperazin-1-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 6) trifluoroacetate

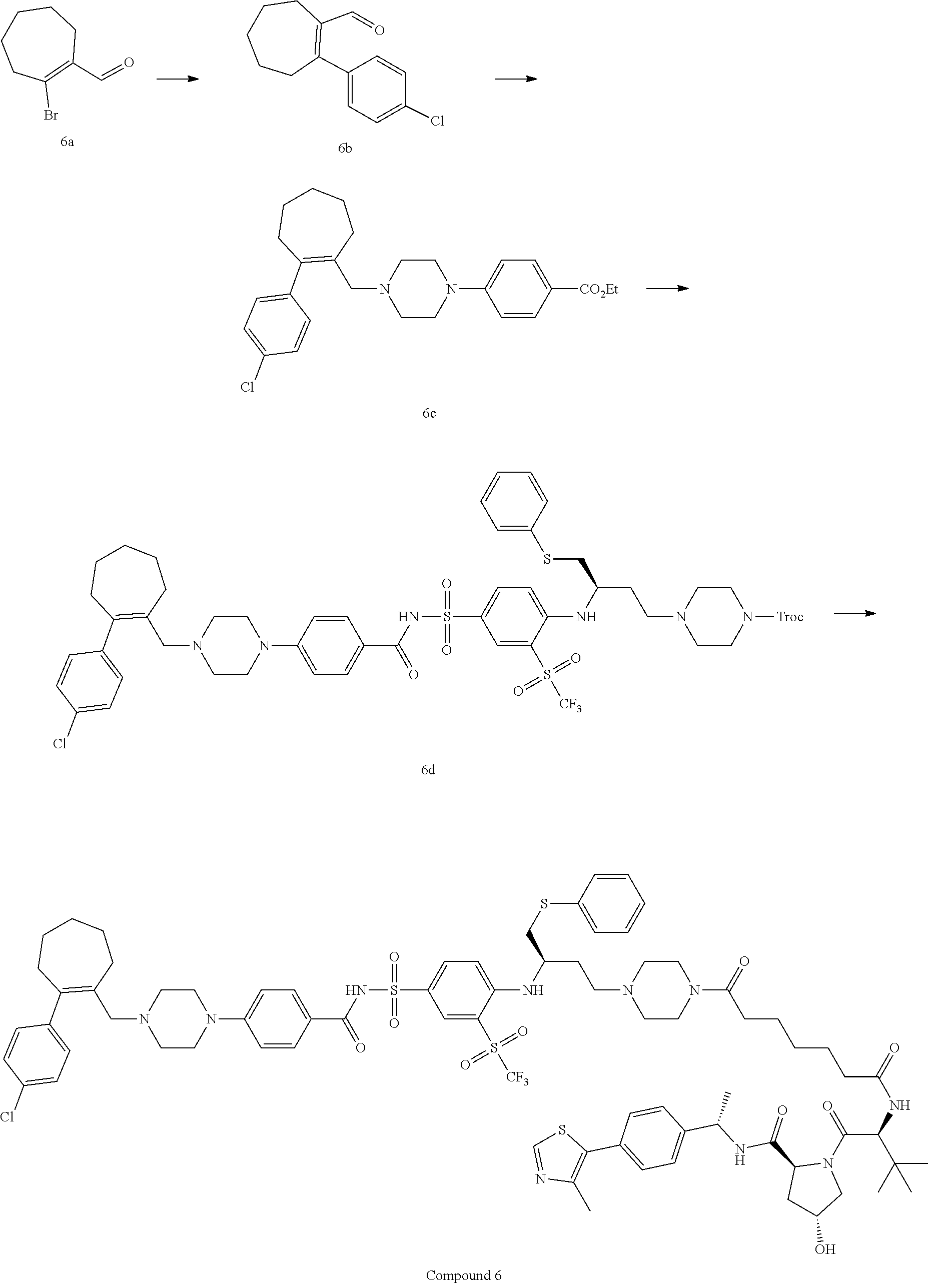

### Step 1: Preparation of 6b

Under nitrogen protection, 6a (see WO 2020041406 for the synthetic method) (2 g, 9.85 mmol) and (4-chlorophenyl)boronic acid (2.47 g, 15.80 mmol) were sequentially dissolved in 20 mL of dioxane and 2 mL of water. Potassium acetate (3.11 g, 31.69 mmol) and Pd(dppf)Cl₂ (0.2 g, 0.27 mmol) were sequentially added and the mixture was reacted at 90° C. for 4 h. The reaction solution was cooled to room temperature and to the reaction solution was slowly added 100 mL of water. The mixture was extracted with ethyl acetate (60 mL×2), dried over anhydrous sodium sulfate and concentrated under reduced pressure and the resulting crude was separated and purified by silica gel column chromatography (ethyl acetate/petroleum ether (v/v)=0:1-1:10) to afford 6b (2.0 g, yield: 87%).

¹H NMR (400 MHz, CDCl₃) δ 9.39 (s, 1H), 7.38-7.30 (m, 2H), 7.18-7.10 (m, 2H), 2.78-2.68 (m, 2H), 2.66-2.56 (m, 2H), 1.95-1.80 (m, 2H), 1.78-1.65 (m, 2H), 1.60-1.47 (m, 2H).

### Step 2: Preparation of 6c

6b (1.75 g, 7.52 mmol) was dissolved in 15 mL of tetrahydrofuran. Ethyl 4-(piperazin-1-yl)benzoate (1.64 g, 7.00 mmol) and 5 mL of tetraisopropyl titanate were sequentially added and the mixture was stirred at room temperature for 4 h. Then sodium triacetoxyborohydride (3.17 g, 14.96 mmol) was added and the resulting mixture was reacted at room temperature for 16 h. To the reaction solution was slowly added 20 mL of saturated sodium bicarbonate solution. The resulting mixture was extracted with ethyl acetate (60 mL×2). The organic phase was washed with 50 mL of saturated sodium chloride solution, dried over anhydrous sodium sulfate and concentrated under reduced pressure and the resulting crude was separated and purified by silica gel column chromatography (ethyl acetate/petroleum ether (v/v)=0:1-1:1) to afford 6c (2.0 g, yield: 63%).

¹H NMR (400 MHz, CDCl₃) δ 7.93-7.86 (m, 2H), 7.30-7.20 (m, 2H), 7.03-6.94 (m, 2H), 6.86-6.74 (m, 2H), 4.31 (q, 2H), 3.33-3.19 (m, 4H), 2.81 (s, 2H), 2.55-2.26 (m, 8H), 1.90-1.75 (m, 2H), 1.67-1.48 (m, 4H), 1.36 (t, 3H).

### Step 3: Preparation of 6d

6c (0.40 g, 0.88 mmol) was dissolved in 10 mL of methanol. Water (1 mL) and sodium hydroxide (0.15 g, 3.75 mmol) were added and the mixture was reacted at 80° C. for 10 h. The reaction solution was cooled to room temperature and concentrated under reduced pressure. The residue was dissolved with 10 mL of water and then extracted with 20 mL of methyl tert-butyl ether to remove the impurities. The aqueous phase was separated, adjusted to pH 6 with 1 mol/L hydrochloric acid, extracted with ethyl acetate (100 mL×2), dried over anhydrous sodium sulfate and concentrated under reduced pressure to afford a crude (0.20 g). The above-mentioned crude (200 mg) was dissolved in 15 mL of DCM. Intermediate 2 (330 mg, 0.45 mmol), DMAP (110 mg, 0.9 mmol) and EDCI (180 mg, 0.94 mmol) were sequentially added and the mixture was reacted at room temperature for 16 h. To the reaction system was slowly added 30 mL of water and the mixture was extracted with DCM (60 mL×2). The organic phase was washed with 50 mL of water, dried over anhydrous sodium sulfate and concentrated under reduced pressure and the resulting crude was separated and purified by silica gel column chromatography (dichloromethane/methanol (v/v)=9:1) to afford 6d (260 mg, yield: 26%).

LCMS m/z=568.2 [M/2+1]⁺.

### Step 4: Preparation of Trifluoroacetate of Compound 6

6d (260 mg, 0.23 mmol) was dissolved in 20 mL of tetrahydrofuran. Acetic acid (0.6 mL) and zinc powder (960 mg, 14.8 mmol) were sequentially added and the mixture was reacted at room temperature for 16 h. The reaction system was filtered and to the filtrate was added water (10 mL). The mixture was then extracted with 20 mL of ethyl acetate. The organic phase was separated, dried over anhydrous sodium sulfate and concentrated under reduced pressure to afford a crude (250 mg). The above-mentioned crude (250 mg) was dissolved in 10 mL of DCM. Triethylamine (0.2 mL), intermediate 1 (154 mg, 0.27 mmol) and HATU (142 mg, 0.37 mmol) were sequentially added and the mixture was reacted at room temperature for 1 h. To the reaction system was added 20 mL of water and the mixture was extracted with 20 mL of ethyl acetate. The organic phase was washed with 20 mL of water, dried over anhydrous sodium sulfate and concentrated under reduced pressure and the resulting crude was passed through Pre-HPLC (instrument and preparative column: using Glison GX-281 preparative liquid phase chromatographic instrument, preparative column model: Sunfire C18, 5 μm, inner diameter×length=30 mm×150 mm). Preparation method: the crude was dissolved with methanol and dimethyl sulfoxide, and filtered with a 0.45 m filter membrane, to prepare into a sample liquid. Mobile phase system: acetonitrile/water (containing 0.1% TFA). Gradient elution method: gradient elution with acetonitrile from 5% to 60% (elution time: 15 min). Lyophilization was performed to afford the trifluoroacetate of compound 6 (20 mg).

¹H NMR (400 MHz, CDCl₃) δ 8.67 (s, 1H), 8.43-8.30 (m, 1H), 8.15-8.05 (m, 1H), 7.74-7.62 (m, 2H), 7.47-7.22 (m, 12H), 7.16-7.05 (m, 1H), 7.02-6.92 (m, 2H), 6.84-6.71 (m, 2H), 6.70-6.55 (m, 1H), 6.34-6.18 (m, 1H), 5.14-5.00 (m, 1H), 4.80-4.67 (m, 1H), 4.64-4.55 (m, 1H), 4.55-4.43 (m, 1H), 4.16-4.04 (m, 1H), 4.00-3.84 (m, 1H), 3.75-3.55 (m, 2H), 3.54-3.20 (m, 7H), 3.17-2.85 (m, 4H), 2.65-2.00 (m, 24H), 1.90-1.75 (m, 2H), 1.66-1.50 (m, 9H), 1.50-1.42 (m, 3H), 1.40-1.25 (m, 2H), 1.04 (s, 9H).

LCMS m/z=764.4 [M/2+1]⁺.

### Synthetic Method of Free-Form Compound 6:

After the reaction in step 4, the system was quenched with water and extracted with ethyl acetate. The organic phase was washed with water, dried over anhydrous sodium sulfate and concentrated under reduced pressure. The resulting crude was subjected to preparative liquid phase chromatography to afford free-form compound 6.

### Preparative Liquid Phase Chromatography Method:

instrument and preparative column: using SHIMADZU LC-20AP preparative liquid phase chromatographic instrument, preparative column model: Phenomenex C18. Preparation method: the crude was dissolved with acetonitrile and water to prepare into a sample liquid. Mobile phase system: acetonitrile/water (containing 10 mmol/L ammonium bicarbonate). Gradient elution method: gradient elution with acetonitrile from 50% to 70% (elution time: 20 min).

### Nuclear Magnetic Resonance of Free-Form Compound 6

¹H NMR (400 MHz, CDCl₃) δ 8.67 (s, 1H), 8.40-8.31 (m, 1H), 8.13-8.04 (m, 1H), 7.78-7.63 (m, 2H), 7.46-7.18 (m, 12H), 7.12-7.02 (m, 1H), 7.02-6.92 (m, 2H), 6.80-6.68 (m, 2H), 6.67-6.56 (m, 1H), 6.43-6.31 (m, 1H), 5.13-4.99 (m, 1H), 4.78-4.67 (m, 1H), 4.66-4.55 (m, 1H), 4.54-4.44 (m, 1H), 4.14-4.02 (m, 1H), 3.98-3.82 (m, 1H), 3.71-3.54 (m, 2H), 3.50-3.18 (m, 7H), 3.16-2.85 (m, 4H), 2.59-1.96 (m, 24H), 1.90-1.75 (m, 2H), 1.75-1.50 (m, 9H), 1.50-1.40 (m, 3H), 1.40-1.16 (m, 2H), 1.04 (s, 9H).

LCMS m/z=764.4 [M/2+1]⁺.

Example 7

6-(4-((4′-chloro-4,4-dimethyl-3,4,5,6-tetrahydro-[1,1′-biphenyl]-2-yl)methyl)piperazin-1-yl)-N-((4-(((R)-4-(4-(7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptanoyl)piperazin-1-yl)-1-(phenylthio)butan-2-yl)amino)-3-((trifluoromethyl)sulfonyl)phenyl)sulfonyl)nicotinamide (Compound 7)

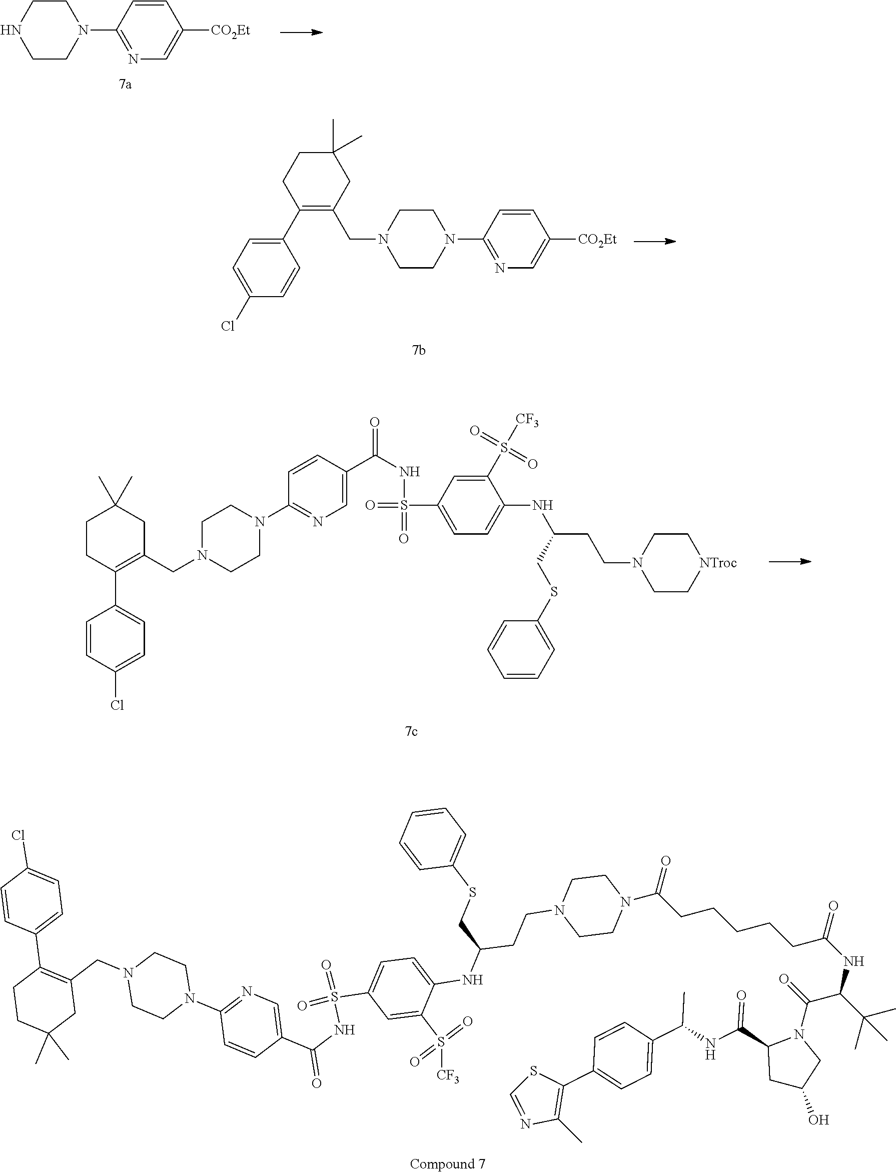

### Step 1: Preparation of 7b

4′-chloro-4,4-dimethyl-3,4,5,6-tetrahydro-[1,1′-biphenyl]-2-carbaldehyde (370 mg, 1.49 mmol) was dissolved in 15 mL of tetrahydrofuran. 7a (see CN 105985321 for the synthetic method) (350 mg, 1.49 mmol) and 1 mL of titanium isopropoxide were sequentially added and the mixture was stirred at room temperature for 4 h. Then sodium triacetoxyborohydride (947 mg, 4.47 mmol) was added and the resulting mixture was stirred at room temperature for 16 h. To the reaction solution was slowly added 20 mL of saturated aqueous sodium bicarbonate solution. The resulting mixture was extracted with 60 mL of ethyl acetate twice and the organic phase was washed with 50 mL of saturated sodium chloride solution, dried over anhydrous sodium sulfate and concentrated under reduced pressure. The resulting crude was separated and purified by silica gel column chromatography (ethyl acetate/petroleum ether (v/v)=0:1-1:1) to afford 7b (320 mg, yield: 46%).

LCMS m/z=468.2 [M+1]⁺.

### Step 2: Preparation of 7c

7b (0.320 g, 0.68 mmol) was dissolved in 10 mL of methanol. Water (1 mL) and sodium hydroxide (0.11 g, 2.75 mmol) were added and the mixture was reacted at 80° C. for 10 h. The reaction system was cooled to room temperature and concentrated under reduced pressure. The residue was dissolved with 10 mL of water and then extracted with 20 mL of methyl tert-butyl ether to remove the impurities. The resulting mixture was adjusted to pH 6 with 1 mol/L hydrochloric acid, extracted with ethyl acetate (100 mL×2), dried over anhydrous sodium sulfate and concentrated under reduced pressure to afford a crude (0.23 g). The above-mentioned crude (230 mg) was dissolved in 15 mL of DCM. Intermediate 2 (390 mg, 0.53 mmol), DMAP (200 mg, 1.64 mmol) and EDCI (130 mg, 0.68 mmol) were sequentially added and the mixture was reacted at room temperature for 16 h. To the reaction system was slowly added 30 mL of water and the mixture was extracted with 60 mL of DCM twice. The organic phase was washed with 50 mL of water, dried over anhydrous sodium sulfate and concentrated under reduced pressure and the resulting crude was separated and purified by silica gel column chromatography (dichloromethane/methanol (v/v)=9:1) to afford 7c (260 mg, yield: 33%).

LCMS m/z=575.8 [½M+1]⁺.

### Step 3: Preparation of Compound 7

7c (260 mg, 0.23 mmol) was dissolved in 20 mL of tetrahydrofuran. Acetic acid (0.6 mL) and zinc powder (960 mg, 14.8 mmol) were sequentially added and the mixture was reacted at room temperature for 16 h. The reaction system was filtered and to the filtrate was added water (10 mL). The mixture was then extracted with 20 mL of ethyl acetate. The organic phase was separated, dried over anhydrous sodium sulfate and concentrated under reduced pressure to afford a crude (190 mg). The above-mentioned crude (190 mg) was dissolved in 10 mL of DCM. Triethylamine (0.2 mL), intermediate 1 (120 mg, 0.21 mmol) and HATU (110 mg, 0.29 mmol) were sequentially added and the mixture was reacted at room temperature for 1 h. To the reaction system was added 20 mL of water and the mixture was extracted with 20 mL of ethyl acetate. The organic phase was washed with 20 mL of water, dried over anhydrous sodium sulfate and concentrated under reduced pressure and the resulting crude was passed through Pre-HPLC (instrument and preparative column: using Glison GX-281 preparative liquid phase chromatographic instrument, preparative column model: Sunfire C18, 5 μm, inner diameter×length=30 mm×150 mm). Preparation method: the crude was dissolved with methanol and dimethyl sulfoxide, and filtered with a 0.45 m filter membrane, to prepare into a sample liquid. Mobile phase system: acetonitrile/water (containing 0.1% TFA). Gradient elution method: gradient elution with acetonitrile from 5% to 60% (elution time: 15 min). To the preparative solution were added 100 mL of DCM and 60 mL of saturated sodium bicarbonate solution and the mixture was stirred for 1 h. The organic phase was separated, dried over anhydrous sodium sulfate and concentrated under reduced pressure to afford compound 7 (60 mg, yield: 17%).

¹H NMR (400 MHz, CDCl₃) δ 8.74-8.62 (m, 2H), 8.38-8.32 (m, 1H), 8.08-7.99 (m, 1H), 7.93-7.84 (m, 1H), 7.53-7.20 (m, 12H), 7.11-7.00 (m, 1H), 6.99-6.92 (m, 2H), 6.67-6.58 (m, 1H), 6.53-6.44 (m, 1H), 6.44-6.35 (m, 1H), 5.15-5.02 (m, 1H), 4.80-4.68 (m, 1H), 4.65-4.55 (m, 1H), 4.53-4.45 (m, 1H), 4.20-4.05 (m, 1H), 3.97-3.80 (m, 1H), 3.75-3.53 (m, 6H), 3.45-3.20 (m, 3H), 3.18-2.80 (m, 4H), 2.55-1.95 (m, 24H), 1.75-1.40 (m, 10H), 1.40-1.20 (m, 2H), 1.04 (s, 9H), 1.00-0.90 (m, 6H).

LCMS m/z=771.8 [M/2+1]⁺.

Example 8

cis-(2S,4R)-1-((2S)-2-(7-(5-((R)-3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)hexahydropyrrolo[3,4-c]pyrrol-2(1H)-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 8)

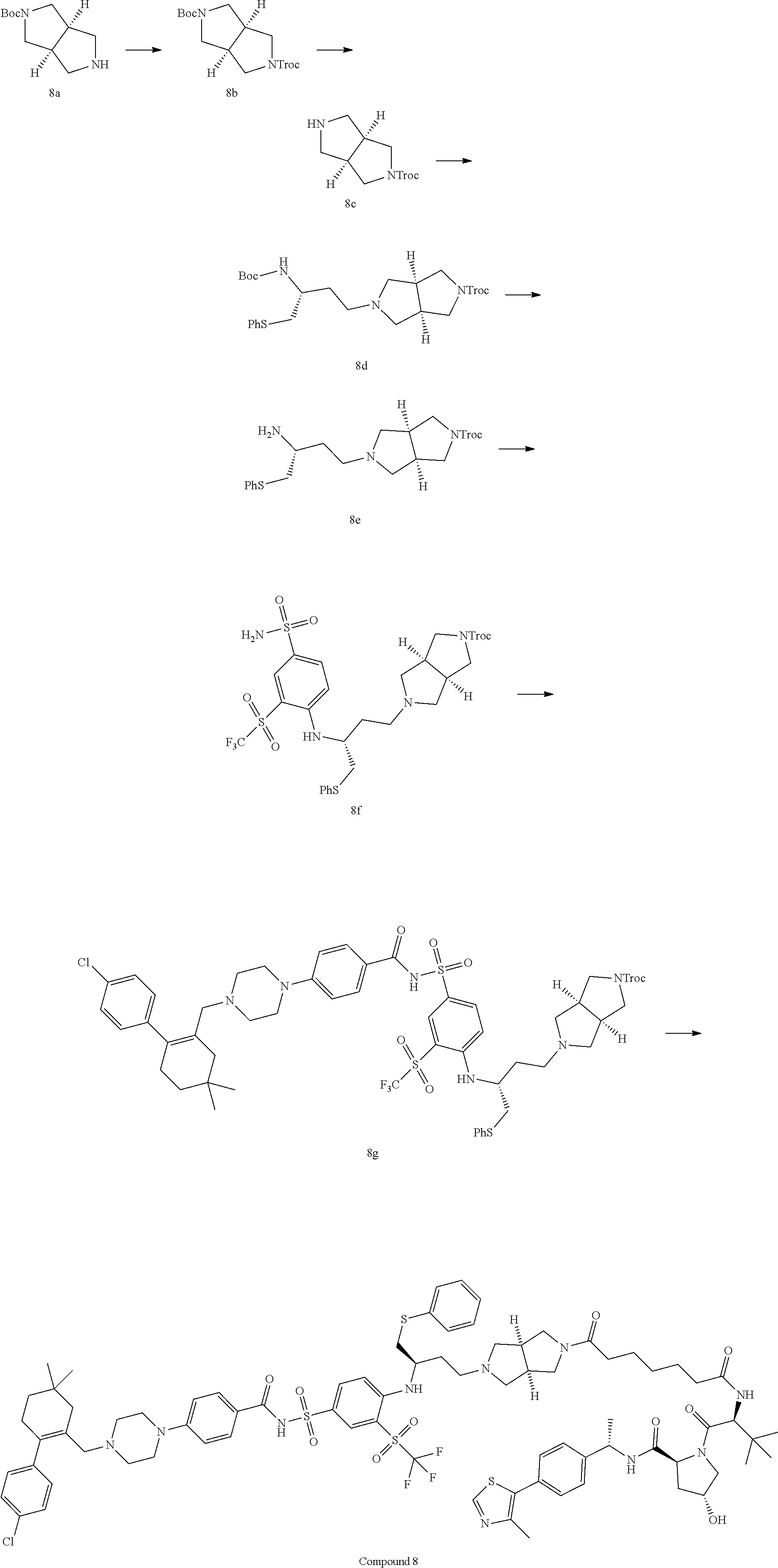

### Step 1: Preparation of 8b

8a (1.88 g, 8.86 mmol) was dissolved in 10 mL of dichloromethane and triethylamine (0.98 g, 9.68 mmol) was added. The mixture was cooled to 0° C. and 2,2,2-trichloroethyl chloroformate (2.06 g, 9.72 mmol) was slowly added dropwise. Then the mixture was slowly warmed to room temperature and reacted for 2 h. After the reaction was completed, the reaction solution was diluted with 50 mL of dichloromethane and then 50 mL of water was added. The organic phase was separated, dried over anhydrous sodium sulfate and concentrated under reduced pressure and the resulting crude was separated and purified by silica gel column chromatography (petroleum ether/ethyl acetate (v/v)=10:1-2:1) to afford 8b (2.4 g, yield: 70%).

### Step 2: Preparation of 8c

8b (0.7 g, 1.81 mmol) was dissolved in 2 mL of dichloromethane. Trifluoroacetic acid (1 mL) was added and the mixture was reacted at room temperature for 3 h. The reaction solution was concentrated under reduced pressure. To the residue were added 20 mL of dichloromethane and 10 mL of water. The mixture was adjusted to pH 9 with saturated sodium bicarbonate solution. The organic phase was separated, dried over anhydrous sodium sulfate and concentrated under reduced pressure to afford crude 8c (0.5 g).

LCMS m/z=287.0 [M+1]⁺.

### Step 3: Preparation of 8d

The above-mentioned crude 8c (0.5 g) was added to 10 mL of 1,2-dichloroethane. Tert-butyl (R)-(4-oxo-1-(phenylthio)butan-2-yl)carbamate (1.03 g, 3.49 mmol) and glacial acetic acid (0.1 mL) were added and the mixture was reacted at room temperature for 0.5 h. Sodium triacetoxyborohydride (0.74 g, 3.49 mmol) was added and the resulting mixture was reacted at room temperature for 19 h. Dichloromethane (50 mL) was added to dilute the reaction solution. Saturated sodium bicarbonate solution was used to adjust the reaction mixture to pH 9. The organic phase was separated, dried over anhydrous sodium sulfate and concentrated under reduced pressure. The resulting crude was separated and purified by chromatographic column on silica gel (dichloromethane:methanol (v/v)=10:1) to afford 8d (0.64 g, yield over two steps from compound 8b: 62%).

LCMS m/z=566.1 [M+1]⁺.

Step 4: Preparation of trifluoroacetate of 8e

8d (0.64 g, 1.13 mmol) was dissolved in 3 mL of dichloromethane. Trifluoroacetic acid (1 mL) was added and the mixture was reacted at room temperature for 3 h. The reaction solution was concentrated under reduced pressure to afford crude trifluoroacetate of 8e (0.6 g).

### Step 5: Preparation of 8f

The above-mentioned crude trifluoroacetate of 8e (0.64 g) was added to 4 mL of acetonitrile. Triethylamine (0.78 mL, 5.61 mmol) and 4-fluoro-3-((trifluoromethyl)sulfonyl)benzenesulfonamide (0.35 g, 1.14 mmol) were added and the mixture was refluxed and reacted at 80° C. for 3 h. The reaction mixture was cooled to room temperature. The reaction solution was concentrated under reduced pressure and the resulting crude was separated and purified by chromatographic column on silica gel (dichloromethane:methanol (v/v)=10:1) to afford 8f (0.88 g, yield over two steps from compound 8d: 97%).

LCMS m/z=753.0 [M+1]⁺.

### Step 6: Preparation of 8g

8f (0.88 g, 1.17 mmol) was added to 10 mL of dichloromethane. 4-(4-((4′-chloro-4,4-dimethyl-3,4,5,6-tetrahydro-[1,1′-biphenyl]-2-yl)methyl)piperazin-1-yl)benzoic acid (0.51 g, 1.16 mmol), DMAP (0.29 g, 2.37 mmol) and EDCI (0.45 g, 2.35 mmol) were sequentially added and the mixture was reacted at room temperature for 12 h. To the reaction system was added 20 mL of water. The organic phase was separated, dried over anhydrous sodium sulfate and concentrated under reduced pressure and the resulting crude was separated and purified by chromatographic column on silica gel (dichloromethane:methanol (v/v)=10:1) to afford 8g (0.85 g, yield: 62%).

LCMS m/z=588.2 [M/2+1]⁺.

### Step 7: Preparation of Compound 8

8g (0.44 g, 0.37 mmol) was added to 30 mL of tetrahydrofuran. Zinc powder (1.26 g, 19.27 mmol) and 0.78 mL of acetic acid were sequentially added and the mixture was reacted at room temperature for 5 h. The reaction system was filtered and the filtrate was concentrated under reduced pressure. The residue was then diluted with 100 mL of DCM and then 50 mL of saturated sodium bicarbonate solution was added. The organic phase was separated, dried over anhydrous sodium sulfate and concentrated under reduced pressure to afford a crude (0.11 g). The above-mentioned crude (0.11 g) was added to 8 mL of DCM. Intermediate 1 (65 mg, 0.11 mmol), triethylamine (0.15 mL, 1.08 mmol) and HATU (63 mg, 0.166 mmol) were sequentially added and the mixture was reacted at room temperature for 2 h. To the reaction system was added 20 mL of water and the mixture was extracted with 30 mL of DCM twice. The organic phase was separated, dried over anhydrous sodium sulfate and concentrated under reduced pressure and the resulting crude was passed through Pre-HPLC (instrument and preparative column: using Glison GX-281 preparative liquid phase chromatographic instrument, preparative column model: Sunfire C18, 5 m, inner diameter×length=30 mm×150 mm). Preparation method: the crude was dissolved with methanol and dimethyl sulfoxide, and filtered with a 0.45 m filter membrane, to prepare into a sample liquid. Mobile phase system: acetonitrile/water (containing 0.1% TFA). Gradient elution method: gradient elution with acetonitrile from 5% to 60% (elution time: 15 min). The preparative solution was lyophilized and 2 mL of ethyl acetate and 1 mL of saturated sodium bicarbonate solution were added to the lyophilized solid. The mixture was stirred for 1 min and the organic phase was separated, dried over anhydrous sodium sulfate and concentrated under reduced pressure to afford compound 8 (18 mg, yield: 10%).

¹H NMR (400 MHz, CDCl₃) δ 8.76-8.62 (m, 1H), 8.54-8.38 (m, 1H), 8.06-7.68 (m, 3H), 7.43-6.90 (m, 15H), 6.87-6.64 (m, 3H), 6.56-6.34 (m, 1H), 5.15-4.87 (m, 1H), 4.85-4.65 (m, 2H), 4.56-4.44 (m, 1H), 4.22-3.84 (m, 2H), 3.75-3.41 (m, 3H), 3.40-2.95 (m, 8H), 2.95-2.55 (m, 6H), 2.55-1.95 (m, 22H), 1.76-1.40 (m, 8H), 1.40-1.20 (m, 4H), 1.15-0.90 (m, 15H).

LCMS m/z=784.5 [M/2+1]⁺.

Example 9

(2S,4R)-1-((S)-2-(7-(2-((R)-3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)-2,7-diazaspiro[3.5]nonan-7-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 9) trifluoroacetate

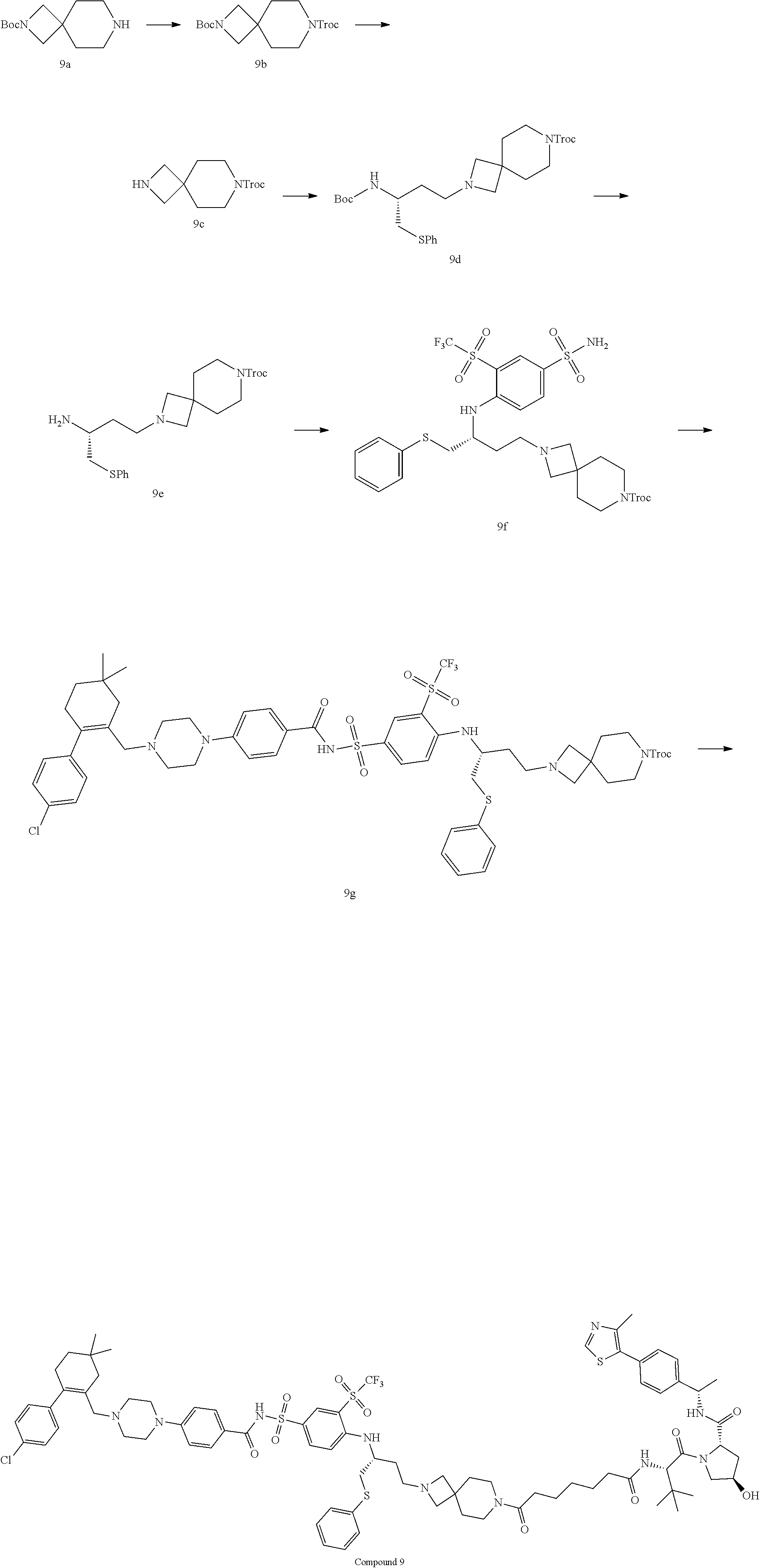

### Step 1: Preparation of 9b

9a (5.00 g, 22.09 mmol) was dissolved in 25 mL of dichloromethane and triethylamine (2.46 g, 24.30 mmol) was added. The mixture was cooled to 0° C. and 2,2,2-trichloroethyl chloroformate (5.15 g, 24.31 mmol) was slowly added dropwise. Then the mixture was slowly warmed to room temperature and reacted for 3 h. After the reaction was completed, the reaction solution was diluted with dichloromethane (50 mL) and then 50 mL of water was added. The organic phase was separated, dried over anhydrous sodium sulfate and concentrated under reduced pressure to afford crude 9b (8.00 g).

### Step 2: Preparation of 9c

The above-mentioned crude 9b (8.00 g) was dissolved in 60 mL of dichloromethane. Trifluoroacetic acid (20 mL) was added and the mixture was reacted at room temperature for 3 h. The reaction solution was concentrated under reduced pressure and the residue was dissolved in 50 mL of dichloromethane. The mixture was adjusted to pH 8 with saturated aqueous sodium bicarbonate solution. Liquid separation was performed. The aqueous phase was extracted with 100 mL of dichloromethane twice, and the organic phases were combined and dried over anhydrous sodium sulfate. The reaction system was filtered and 50 mL of dichloromethane was added to the filter cake. Saturated sodium bicarbonate solution (50 mL) was added and the mixture was stirred until the solid was completely dissolved. The organic phase was separated, dried over anhydrous sodium sulfate and concentrated under reduced pressure to afford crude 9c (3.50 g).

LCMS m/z=301.1 [M+1]⁺.

### Step 3: Preparation of 9d

The above-mentioned crude 9c (0.51 g) was dissolved in 20 mL of DCM. The mixture was cooled to 0° C. Tert-butyl (R)-(4-oxo-1-(phenylthio)butan-2-yl)carbamate (0.50 g, 1.69 mmol) and triethylamine (0.68 g, 6.72 mmol) were added and the mixture was reacted at 0° C. for 15 min. Sodium triacetoxyborohydride (0.54 g, 2.55 mmol) was added and the resulting mixture was reacted at room temperature for 12 h. The reaction solution was concentrated under reduced pressure and the resulting crude was separated and purified by chromatographic column on silica gel (dichloromethane:methanol (v/v)=20:1) to afford 9d (0.60 g, yield: 61%).

LCMS m/z=580.2 [M+1]⁺.

### Step 4: Preparation of 9e

9d (0.60 g, 1.03 mmol) was dissolved in 6 mL of dichloromethane. Trifluoroacetic acid (2 mL) was added and the mixture was reacted at room temperature for 3 h. The reaction solution was concentrated under reduced pressure to afford crude 9e (0.5 g).

LCMS m/z=480.1 [M+1]⁺.

### Step 5: Preparation of 9f

The above-mentioned crude 9e (0.5 g) was added to 10 mL of acetonitrile. DIPEA (0.54 g, 4.18 mmol) and 4-fluoro-3-((trifluoromethyl)sulfonyl) benzenesulfonamide (0.32 g, 1.04 mmol) were added and the mixture was refluxed and reacted at 85° C. for 12 h. The reaction mixture was cooled to room temperature. The reaction solution was concentrated under reduced pressure and the resulting crude was separated and purified by chromatographic column on silica gel (dichloromethane:methanol (v/v)=10:1) to afford 9f (0.2 g, yield over two steps from compound 9d: 25%).

LCMS m/z=767.0 [M+1]⁺.

### Step 6: Preparation of 9g

9f (0.18 g, 0.23 mmol) was added to 6 mL of DCM. 4-(4-((4′-chloro-4,4-dimethyl-3,4,5,6-tetrahydro-[1,1′-biphenyl]-2-yl)methyl)piperazin-1-yl)benzoic acid (0.10 g, 0.23 mmol), DMAP (56 mg, 0.46 mmol) and EDCI (88 mg, 0.46 mmol) were sequentially added and the mixture was reacted at 40° C. for 12 h. The reaction mixture was cooled to room temperature. To the reaction system was added 5 mL of water. Liquid separation was performed. The aqueous phase was extracted with 5 mL of DCM twice. The organic phases were combined, dried over anhydrous sodium sulfate and concentrated under reduced pressure and the crude was separated and purified by chromatographic column on silica gel (dichloromethane:methanol (v/v)=10:1) to afford 9g (0.2 g, yield: 73%).

LCMS m/z=1189.0 [M+1]⁺.

Step 7: Preparation of trifluoroacetate of Compound 9

9g (0.15 g, 0.13 mmol) was added to 12 mL of tetrahydrofuran. Zinc powder (0.56 g, 8.56 mmol) and 0.36 mL of acetic acid were sequentially added and the mixture was reacted at room temperature for 12 h. To the reaction solution was added 5 mL of water to quench the reaction. The mixture was filtered over diatomaceous earth and the filtrate was adjusted to pH 8 with saturated aqueous sodium bicarbonate solution and extracted with 6 mL of dichloromethane twice. The organic phases were combined, dried over anhydrous sodium sulfate and concentrated under reduced pressure. The residue was diluted with 100 mL of dichloromethane and 50 mL of saturated aqueous sodium bicarbonate solution was added. The organic phase was separated, dried over anhydrous sodium sulfate and concentrated under reduced pressure and the resulting crude was separated and purified by chromatographic column on silica gel (dichloromethane:methanol:triethylamine (v/v/v)=100:10:1) to afford a crude (0.06 g). The above-mentioned crude (0.06 g) was added to 3 mL of dichloromethane. Intermediate 1 (0.042 g, 0.071 mmol), triethylamine (0.03 g, 0.3 mmol) and HATU (0.027 g, 0.071 mmol) were sequentially added and the mixture was reacted at room temperature for 2 h. To the reaction solution was added 5 mL of water and liquid separation was performed. The aqueous phase was extracted with 6 mL of dichloromethane twice. The organic phases were combined, dried over anhydrous sodium sulfate and concentrated under reduced pressure and the resulting crude was passed through Pre-HPLC (instrument and preparative column: using Glison GX-281 preparative liquid phase chromatographic instrument, preparative column model: Sunfire C18, 5 μm, inner diameter×length=30 mm×150 mm). Preparation method: the crude was dissolved with methanol and dimethyl sulfoxide, and filtered with a 0.45 m filter membrane, to prepare into a sample liquid. Mobile phase system: acetonitrile/water (containing 0.1% TFA). Gradient elution method: gradient elution with acetonitrile from 5% to 60% (elution time: 15 min). Lyophilization was performed to afford the trifluoroacetate of compound 9 (0.02 g).

¹H NMR (400 MHz, CDCl₃) δ 8.66 (s, 1H), 8.30 (s, 1H), 8.15-7.97 (m, 1H), 7.90-7.70 (m, 2H), 7.67-7.50 (m, 1H), 7.43-7.26 (m, 7H), 7.25-7.12 (m, 4H), 7.05-6.86 (m, 3H), 6.80-6.60 (m, 3H), 6.28-6.05 (m, 1H), 5.16-5.03 (m, 1H), 4.74-4.60 (m, 1H), 4.51-4.36 (m, 2H), 4.16-4.05 (m, 1H), 3.94-3.80 (m, 1H), 3.58-3.47 (m, 1H), 3.45-2.70 (m, 16H), 2.60-2.45 (m, 5H), 2.40-2.15 (m, 10H), 2.12-1.95 (m, 6H), 1.70-1.40 (m, 13H), 1.38-1.25 (m, 2H), 1.10-0.90 (m, 15H).

LCMS m/z=791.6 [M/2+1]⁺.

Example 10

(2S,4R)-1-((S)-2-(7-((1R,4R)-5-((R)-3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)-2,5-diazabicyclo[2.2.1]heptan-2-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 10)

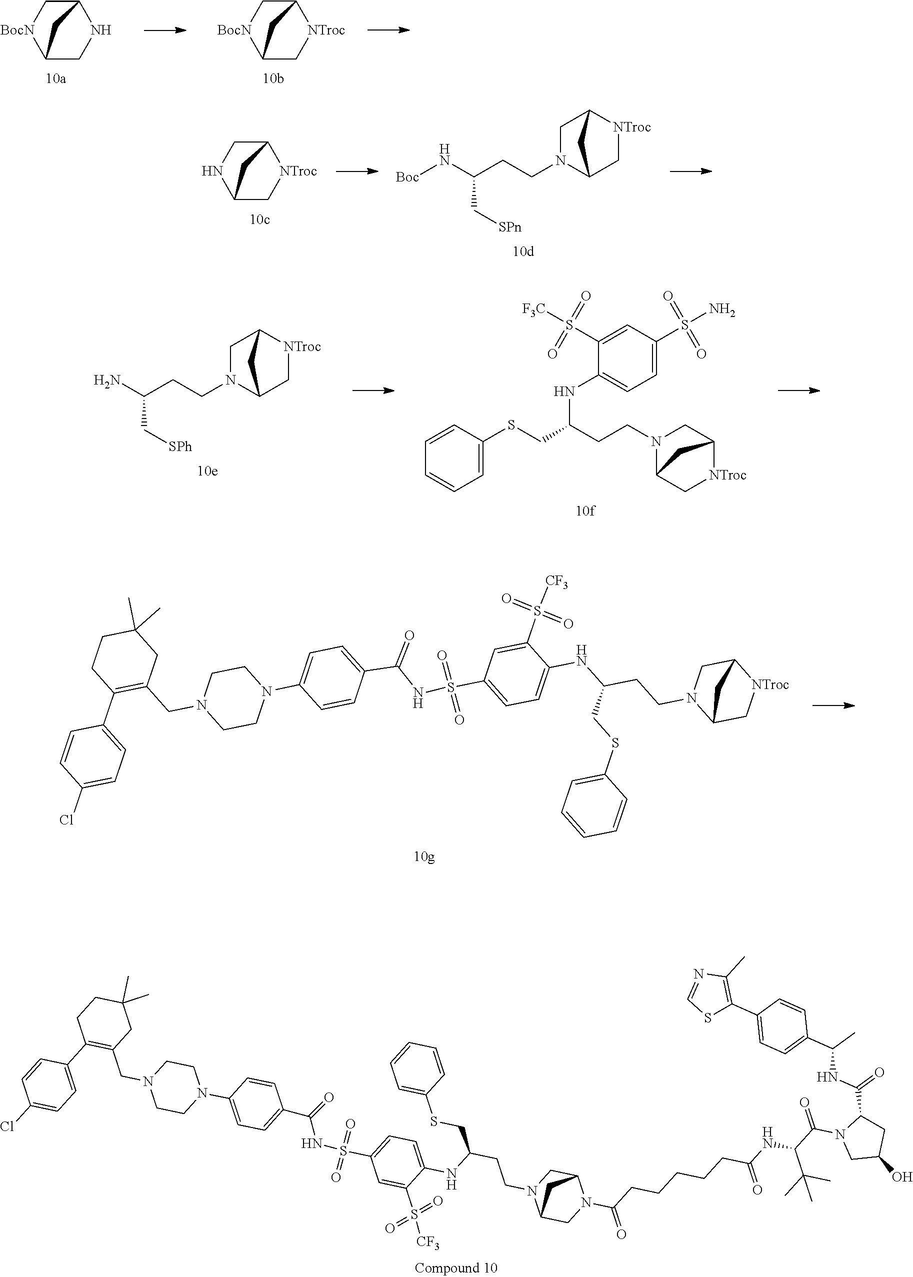

### Step 1: Preparation of 10b

10a (1.90 g, 9.58 mmol) was dissolved in 10 mL of dichloromethane and triethylamine (1.07 g, 10.57 mmol) was added. The mixture was cooled to 0° C. and 2,2,2-trichloroethyl chloroformate (2.24 g, 10.57 mmol) was slowly added dropwise. Then the mixture was slowly warmed to room temperature and reacted for 3 h. After the reaction was completed, the reaction was quenched with 15 mL of water and liquid separation was performed. The aqueous phase was extracted with 15 mL of dichloromethane. The organic phases were combined, dried over anhydrous sodium sulfate and concentrated under reduced pressure to afford crude 10b (3.25 g).

### Step 2: Preparation of 10c

The above-mentioned crude 10b (3.25 g) was dissolved in 20 mL of DCM. Trifluoroacetic acid (7 mL) was added and the mixture was reacted at room temperature for 3 h. The reaction solution was concentrated under reduced pressure and the residue was dissolved with 50 mL of DCM and adjusted to pH 8 with saturated aqueous sodium bicarbonate solution. Liquid separation was performed. The aqueous phase was extracted with 100 mL of DCM twice and the organic phases were combined, dried over anhydrous sodium sulfate and concentrated under reduced pressure to afford crude 10c (2.30 g).

### Step 3: Preparation of 10d

The above-mentioned crude 10c (0.93 g) was dissolved in 20 mL of DCM. The mixture was cooled to 0° C. Tert-butyl (R)-(4-oxo-1-(phenylthio)butan-2-yl)carbamate (1.00 g, 3.38 mmol) and triethylamine (1.37 g, 13.54 mmol) were added and the mixture was reacted at 0° C. for 15 min. Sodium triacetoxyborohydride (1.08 g, 5.10 mmol) was added and the resulting mixture was reacted at room temperature for 12 h. The reaction solution was concentrated under reduced pressure and the resulting crude was separated and purified by chromatographic column on silica gel (dichloromethane:methanol (v/v)=20:1) to afford 10d (1.20 g, yield: 64%).

LCMS m/z=552.1 [M+1]⁺.

### Step 4: Preparation of Trifluoroacetate of 10e

10d (1.20 g, 2.18 mmol) was dissolved in 9 mL of DCM. Trifluoroacetic acid (3 mL) was added and the mixture was reacted at room temperature for 3 h. The reaction solution was concentrated under reduced pressure to afford crude trifluoroacetate of 10e (1.48 g).

### Step 5: Preparation of 10f

The above-mentioned crude trifluoroacetate of 10e (1.48 g) was added to 10 mL of acetonitrile. DIPEA (1.40 g, 10.83 mmol) and 4-fluoro-3-((trifluoromethyl)sulfonyl)benzenesulfonamide (0.67 g, 2.18 mmol) were added and the mixture was refluxed and reacted at 85° C. for 12 h. The reaction mixture was cooled to room temperature. The reaction solution was concentrated under reduced pressure and the resulting crude was separated and purified by chromatographic column on silica gel (dichloromethane:methanol (v/v)=10:1) to afford 10f (1.46 g, yield over two steps from compound 10d: 91%).

LCMS m/z=739.0 [M+1]⁺.

### Step 6: Preparation of 10g

10f (0.66 g, 0.89 mmol) was added to 6 mL of DCM. 4-(4-((4′-chloro-4,4-dimethyl-3,4,5,6-tetrahydro-[1,1′-biphenyl]-2-yl)methyl)piperazin-1-yl)benzoic acid (0.39 g, 0.90 mmol), DMAP (0.22 g, 1.81 mmol) and EDCI (0.34 g, 1.78 mmol) were sequentially added and the mixture was reacted at 35° C. for 12 h. The reaction mixture was cooled to room temperature. To the reaction system were added 5 mL of water and 3 mL of DCM. Liquid separation was performed. The aqueous phase was extracted with 5 mL of DCM twice. The organic phases were combined, dried over anhydrous sodium sulfate and concentrated under reduced pressure and the crude was separated and purified by chromatographic column on silica gel (dichloromethane:methanol (v/v)=10:1) to afford 10g (0.77 g, yield: 75%).

LCMS m/z=581.2 [M/2+1]⁺.

### Step 7: Preparation of Compound 10

10g (0.77 g, 0.66 mmol) was added to 60 mL of tetrahydrofuran. Zinc powder (2.28 g, 34.85 mmol) and 1.42 mL of acetic acid were sequentially added and the mixture was reacted at room temperature for 4 h. To the reaction solution was added 25 mL of water to quench the reaction. The mixture was filtered over diatomaceous earth and the filtrate was adjusted to pH 8 with saturated aqueous sodium bicarbonate solution and extracted with 6 mL of DCM twice. The organic phases were combined, dried over anhydrous sodium sulfate and concentrated under reduced pressure and the resulting crude was separated and purified by chromatographic column on silica gel (dichloromethane:methanol:triethylamine (v/v/v)=100:10:1) to afford a crude (0.33 g). The above-mentioned crude (0.33 g) was added to 5 mL of DCM. Intermediate 1 (0.21 g, 0.36 mmol), triethylamine (0.17 g, 1.68 mmol) and HATU (0.19 g, 0.5 mmol) were sequentially added and the mixture was reacted at room temperature for 2 h. To the reaction solution was added 5 mL of water and liquid separation was performed. The aqueous phase was extracted with 6 mL of DCM twice and the organic phases were combined, dried over anhydrous sodium sulfate and concentrated under reduced pressure. The resulting crude was preliminarily separated and purified by chromatographic column on silica gel (dichloromethane:methanol (v/v)=10:1) to afford a crude, which was then passed through Pre-HPLC (instrument and preparative column: using SHIMADZU LC-20AP preparative liquid phase chromatographic instrument, preparative column model: Phenomenex C18). Mobile phase system: acetonitrile/water containing 10 mmol/L NH₄HCO₃. Gradient elution method: gradient elution with acetonitrile from 51% to 81% (elution time: 15 min). Lyophilization was performed to afford compound 10 (0.13 g, yield: 13%).

¹H NMR (400 MHz, CDCl₃) δ 8.66 (s, 1H), 8.41-8.32 (m, 1H), 8.14-7.92 (m, 1H), 7.80-7.62 (m, 2H), 7.44-7.26 (m, 10H), 7.26-7.10 (m, 3H), 7.04-6.92 (m, 2H), 6.84-6.70 (m, 2H), 6.68-6.53 (m, 1H), 6.40-6.20 (m, 1H), 5.15-5.00 (m, 1H), 4.82-3.80 (m, 6H), 3.67-2.95 (m, 10H), 2.93-2.55 (m, 4H), 2.55-1.88 (m, 20H), 1.85-1.40 (m, 12H), 1.38-1.20 (m, 2H), 1.10-0.90 (m, 15H).

LCMS m/z=518.8 [M/3+1]⁺.

Example 11

(2S,4R)-1-((S)-2-(7-(4-((R)-3-((4-(N-(4-(4-((6-(4-chlorophenyl)spiro[2.5]oct-5-en-5-yl)methyl)piperazin-1-yl)benzoyl)sulfamoyl)-2-((trifluoromethyl)sulfonyl)phenyl)amino)-4-(phenylthio)butyl)piperazin-1-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 11)

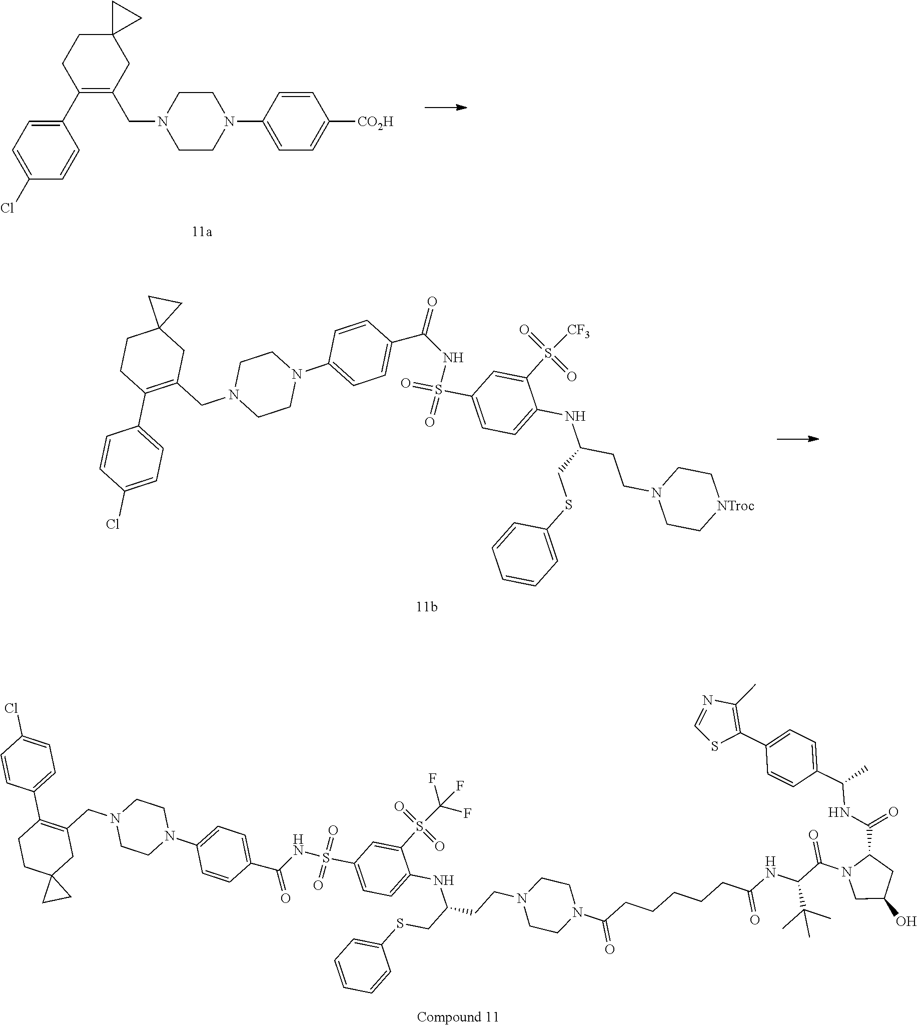

### Step 1: Preparation of 11b

11a (see WO 2013185202 for the synthetic method) (350 mg, 0.8 mmol) was dissolved in 15 mL of DCM. Intermediate 2 (583 mg, 0.79 mmol), DMAP (195 mg, 1.6 mmol) and EDCI (306 mg, 1.6 mmol) were sequentially added and the mixture was reacted at room temperature for 16 h. To the reaction system was slowly added 100 mL of water and the mixture was extracted with 60 mL of DCM twice. The organic phase was dried over anhydrous sodium sulfate and concentrated under reduced pressure and the resulting crude was separated and purified by silica gel column chromatography (dichloromethane/methanol (v/v)=9:1) to afford 11b (550 mg, yield: 60%).

LCMS m/z=574.1 [M/2+1]⁺.

### Step 2: Preparation of Compound 11

11b (260 mg, 0.23 mmol) was dissolved in 20 mL of tetrahydrofuran. Acetic acid (0.6 mL) and zinc powder (960 mg, 14.8 mmol) were sequentially added and the mixture was reacted at room temperature for 16 h. The reaction system was filtered and to the filtrate was added water (10 mL). The mixture was then extracted with 20 mL of ethyl acetate. The organic phase was separated, dried over anhydrous sodium sulfate and concentrated under reduced pressure. The residue was separated and purified by chromatographic column on silica gel (methanol/dichloromethane/triethylamine (v/v)=19:1:0-89:10:1) to afford a crude (160 mg). The above-mentioned crude (160 mg) was dissolved in 15 mL of DCM. Triethylamine (0.22 mL), intermediate 1 (95 mg, 0.17 mmol) and HATU (110 mg, 0.29 mmol) were sequentially added and the mixture was reacted at room temperature for 2 h. To the reaction solution was added 100 mL of water. The mixture was extracted with 50 mL of DCM, dried over anhydrous sodium sulfate and concentrated under reduced pressure. The residue was separated and purified by silica gel column chromatography (methanol/dichloromethane (v/v)=0:1-1:9) and the resulting crude was passed through Pre-HPLC (instrument and preparative column: using SHIMADZU LC-20AP preparative liquid phase chromatographic instrument, preparative column model: Phenomenex C18, mobile phase system: acetonitrile/water containing 10 mmol/L NH₄HCO₃. Gradient elution method: gradient elution with acetonitrile from 58% to 88% (elution time: 10 min)). Lyophilization was performed to afford compound 11 (50 mg, yield: 14%).

¹H NMR (400 MHz, CDCl₃) δ 8.67 (s, 1H), 8.37-8.30 (m, 1H), 8.09 (dd, 1H), 7.71 (d, 2H), 7.44-7.19 (m, 12H), 7.13-6.97 (m, 3H), 6.75 (d, 2H), 6.62 (d, 1H), 6.39 (d, 1H), 5.15-5.00 (m, 1H), 4.80-4.67 (m, 1H), 4.61 (d, 1H), 4.55-4.43 (m, 1H), 4.14-4.02 (m, 1H), 3.99-3.82 (m, 1H), 3.75-3.53 (m, 2H), 3.50-3.17 (m, 7H), 3.16-2.95 (m, 2H), 2.89 (s, 2H), 2.57-1.97 (m, 24H), 1.76-1.40 (m, 10H), 1.38-1.22 (m, 2H), 1.04 (s, 9H), 0.35 (s, 4H).

LCMS m/z=770.8 [M/2+1]⁺.

Example 12

(2S,4R)-1-((S)-2-(7-(4-((R)-3-((4-(N-(4-(4-((4′-chloro-2′-fluoro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazin-1-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 12)

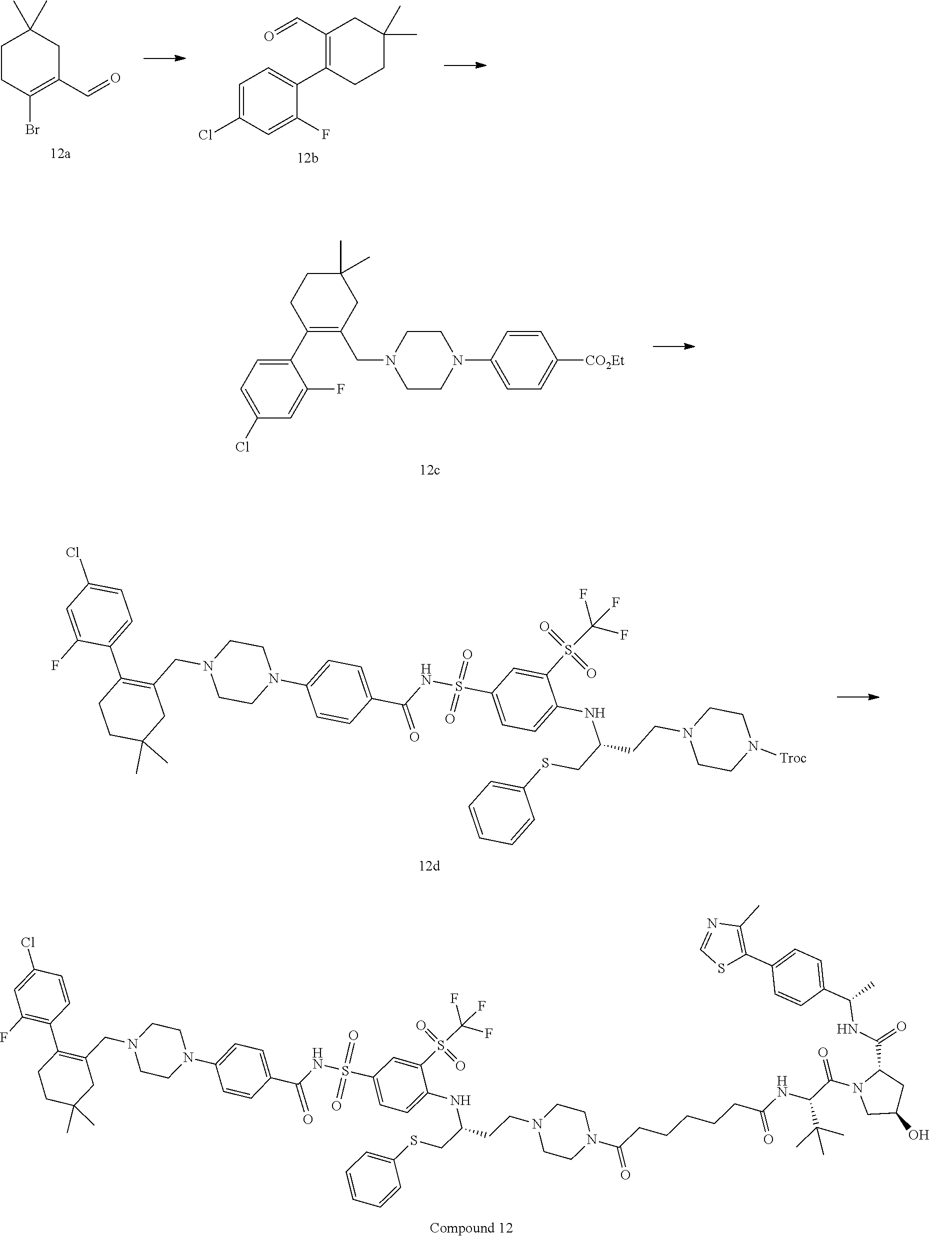

### Step 1: Preparation of 12b

Under nitrogen protection, 12a (see WO 2021066873 for the synthetic method) (2 g, 9.21 mmol) and (4-chloro-2-fluorophenyl)boronic acid (2.41 g, 13.82 mmol) were dissolved in 20 mL of dioxane and 2 mL of water. Potassium acetate (2.71 g, 27.61 mmol) and Pd(dppf)Cl₂ (0.2 g, 0.27 mmol) were sequentially added and the mixture was reacted at 90° C. for 4 h. The reaction solution was cooled to room temperature and to the reaction solution was slowly added 100 mL of water. The mixture was extracted with ethyl acetate (60 mL×2), dried over anhydrous sodium sulfate and concentrated under reduced pressure and the resulting crude was separated and purified by silica gel column chromatography (ethyl acetate/petroleum ether (v/v)=0:1-1:10) to afford 12b (2.0 g, yield: 81%).

### Step 2: Preparation of 12c

12b (2.4 g, 9.0 mmol) was dissolved in 15 mL of DCE. Ethyl 4-(piperazin-1-yl)benzoate (2.10 g, 9.0 mmol) and 5 mL of tetraisopropyl titanate were sequentially added and the mixture was stirred at room temperature for 3 h. Then sodium triacetoxyborohydride (3.81 g, 17.98 mmol) was added and the resulting mixture was reacted at room temperature for 16 h. To the reaction solution was slowly added 20 mL of saturated sodium bicarbonate solution. The resulting mixture was extracted with ethyl acetate (60 mL×2). The organic phase was washed with 50 mL of saturated sodium chloride solution, dried over anhydrous sodium sulfate and concentrated under reduced pressure and the resulting crude was separated and purified by silica gel column chromatography (ethyl acetate/petroleum ether (v/v)=0:1-1:1) to afford 12c (1.0 g, yield: 23%).

LCMS m/z=485.2 [M+1]⁺.

### Step 3: Preparation of 12d

12c (1.4 g, 2.89 mmol) was dissolved in 10 mL of methanol. Water (1 mL) and sodium hydroxide (0.35 g, 8.75 mmol) were added and the mixture was reacted at 80° C. for 10 h. The reaction solution was cooled to room temperature and concentrated under reduced pressure. The residue was dissolved with 10 mL of water and then washed with 20 mL of methyl tert-butyl ether. The aqueous phase was separated, adjusted to pH 6 with 1 mol/L hydrochloric acid, extracted with ethyl acetate (100 mL×2), dried over anhydrous sodium sulfate and concentrated under reduced pressure to afford a crude (0.8 g). The above-mentioned crude (0.4 g) was dissolved in 15 mL of DCM. Intermediate 2 (0.64 g, 0.87 mmol), DMAP (320 mg, 2.62 mmol) and EDCI (340 mg, 1.77 mmol) were sequentially added and the mixture was reacted at room temperature for 16 h. To the reaction system was slowly added 30 mL of water and the mixture was extracted with DCM (60 mL×2). The organic phase was washed with 50 mL of water, dried over anhydrous sodium sulfate and concentrated under reduced pressure and the resulting crude was separated and purified by silica gel column chromatography (dichloromethane/methanol (v/v)=9:1) to afford 12d (0.6 g, yield: 59%).

LCMS m/z=584.1 [M/2+1]⁺.

### Step 4: Preparation of Compound 12

12d (0.5 g, 0.43 mmol) was dissolved in 20 mL of tetrahydrofuran. Acetic acid (1.6 g, 26.64 mmol) and zinc powder (1.6 g, 24.67 mmol) were sequentially added and the mixture was reacted at room temperature for 16 h. The reaction system was filtered and to the filtrate was added water (10 mL). The mixture was then extracted with 20 mL of ethyl acetate. The organic phase was separated, dried over anhydrous sodium sulfate and concentrated under reduced pressure to afford a crude (0.2 g). The above-mentioned crude (0.2 g) was dissolved in 10 mL of DCM. Triethylamine (0.2 mL), intermediate 1 and HATU (0.15 g, 0.4 mmol) were sequentially added and the mixture was reacted at room temperature for 1 h. To the reaction system was added 20 mL of water and the mixture was extracted with 20 mL of ethyl acetate. The organic phase was washed with 20 mL of water, dried over anhydrous sodium sulfate and concentrated under reduced pressure and the resulting crude was passed through Pre-HPLC (instrument and preparative column: using SHIMADZU LC-20AP preparative liquid phase chromatographic instrument, preparative column model: Phenomenex C18). Mobile phase system: acetonitrile/water containing 10 mmol/L NH₄HCO₃. Gradient elution method: gradient elution with acetonitrile from 54% to 74% (elution time: 15 min). Lyophilization was performed to afford compound 12 (80 mg, yield: 12%).

¹H NMR (400 MHz, CDCl₃) δ 8.67 (s, 1H), 8.38-8.32 (m, 1H), 8.09 (dd, 1H), 7.70 (d, 2H), 7.45-7.18 (m, 10H), 7.15-6.87 (m, 4H), 6.84-6.70 (m, 2H), 6.62 (d, 1H), 6.34 (d, 1H), 5.15-5.00 (m, 1H), 4.80-4.68 (m, 1H), 4.66-4.56 (m, 1H), 4.54-4.44 (m, 1H), 4.15-4.03 (m, 1H), 3.98-3.82 (m, 1H), 3.73-3.53 (m, 2H), 3.51-3.20 (m, 7H), 3.16-2.93 (m, 2H), 2.75 (s, 2H), 2.55-1.95 (m, 24H), 1.80-1.43 (m, 10H), 1.40-1.22 (m, 2H), 1.10-0.94 (m, 15H).

LCMS m/z=520.8 [M/3+1]⁺.

Example 13

(2S,4R)-1-((S)-2-(7-((1S,4S)-5-((R)-3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)-2,5-diazabicyclo[2.2.1]heptan-2-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 13)

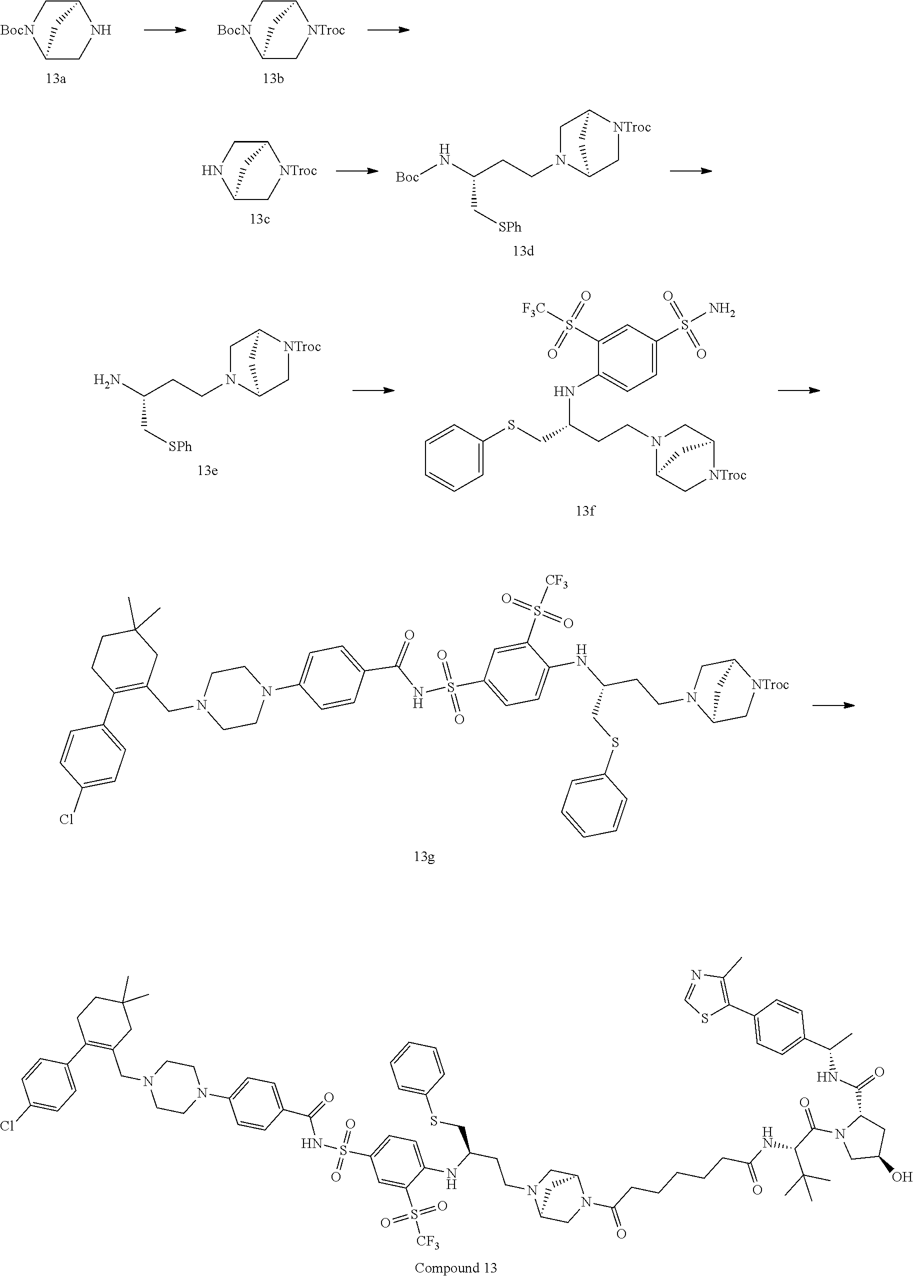

### Step 1: Preparation of 13b

13a (1.90 g, 9.58 mmol) was dissolved in 10 mL of DCM and triethylamine (1.07 g, 10.57 mmol) was added. The mixture was cooled to 0° C. and 2,2,2-trichloroethyl chloroformate (2.24 g, 10.57 mmol) was slowly added dropwise. Then the mixture was slowly warmed to room temperature and reacted for 3 h. After the reaction was completed, the reaction was quenched with 15 mL of water and liquid separation was performed. The aqueous phase was extracted with 15 mL of DCM. The organic phases were combined, dried over anhydrous sodium sulfate and concentrated under reduced pressure to afford crude 13b (3.25 g).

### Step 2: Preparation of 13c

The above-mentioned crude 13b (3.0 g) was dissolved in 20 mL of DCM. Trifluoroacetic acid (7 mL) was added and the mixture was reacted at room temperature for 3 h. The reaction solution was concentrated under reduced pressure and the residue was dissolved with 50 mL of DCM and adjusted to pH 8 with saturated aqueous sodium bicarbonate solution. Liquid separation was performed. The aqueous phase was extracted with 100 mL of DCM twice and the organic phases were combined, dried over anhydrous sodium sulfate and concentrated under reduced pressure to afford crude 13c (2.0 g).

### Step 3: Preparation of 13d

The above-mentioned crude 13c (1.00 g) was dissolved in 20 mL of DCM. The mixture was cooled to 0° C. Tert-butyl (R)-(4-oxo-1-(phenylthio)butan-2-yl)carbamate (1.08 g, 3.65 mmol) and triethylamine (1.37 g, 13.54 mmol) were added and the mixture was reacted at 0° C. for 15 min. Sodium triacetoxyborohydride (1.08 g, 5.10 mmol) was added and the resulting mixture was reacted at room temperature for 12 h. The reaction solution was concentrated under reduced pressure and the resulting crude was separated and purified by chromatographic column on silica gel (dichloromethane:methanol (v/v)=20:1) to afford 13d (1.2 g, yield: 59%).

### Step 4: Preparation of Trifluoroacetate of 13e

13d (1.20 g, 2.18 mmol) was dissolved in 9 mL of DCM. Trifluoroacetic acid (3 mL) was added and the mixture was reacted at room temperature for 3 h. The reaction solution was concentrated under reduced pressure to afford crude trifluoroacetate of 13e (1.48 g).

### Step 5: Preparation of 13f

The above-mentioned crude trifluoroacetate of 13e (1.48 g) was added to 10 mL of acetonitrile. DIPEA (1.40 g, 10.83 mmol) and 4-fluoro-3-((trifluoromethyl)sulfonyl)benzenesulfonamide (0.67 g, 2.18 mmol) were added and the mixture was refluxed and reacted at 85° C. for 12 h. The reaction mixture was cooled to room temperature. The reaction solution was concentrated under reduced pressure and the resulting crude was separated and purified by chromatographic column on silica gel (dichloromethane:methanol (v/v)=10:1) to afford 13f (1.46 g, yield over two steps from compound 13d: 91%).

LCMS m/z=739.0 [M+1]⁺.

### Step 6: Preparation of 13g

13f (0.7 g, 0.95 mmol) was added to 6 mL of DCM. 4-(4-((4′-chloro-4,4-dimethyl-3,4,5,6-tetrahydro-[1,1′-biphenyl]-2-yl)methyl)piperazin-1-yl)benzoic acid (0.42 g, 0.97 mmol), DMAP (0.35 g, 2.88 mmol) and EDCI (0.36 g, 1.88 mmol) were sequentially added and the mixture was reacted at 35° C. for 12 h. The reaction mixture was cooled to room temperature. To the reaction system were added 5 mL of water and 3 mL of DCM. Liquid separation was performed. The aqueous phase was extracted with 5 mL of DCM twice. The organic phases were combined, dried over anhydrous sodium sulfate and concentrated under reduced pressure and the crude was separated and purified by chromatographic column on silica gel (dichloromethane:methanol (v/v)=10:1) to afford 13g (0.5 g, yield: 45%).

LCMS m/z=581.2 [M/2+1]⁺.

### Step 7: Preparation of Compound 13

13g (0.5 g, 0.43 mmol) was added to 60 mL of tetrahydrofuran. Zinc powder (1.6 g, 24.46 mmol) and 1.6 mL of acetic acid were sequentially added and the mixture was reacted at room temperature for 4 h. To the reaction solution was added 25 mL of water to quench the reaction. The mixture was filtered over diatomaceous earth and the filtrate was adjusted to pH 8 with saturated aqueous sodium bicarbonate solution and extracted with 6 mL of DCM twice. The organic phases were combined, dried over anhydrous sodium sulfate and concentrated under reduced pressure and the resulting crude was separated and purified by chromatographic column on silica gel (dichloromethane:methanol:triethylamine (v/v)=100:10:1) to afford a crude (0.2 g). The above-mentioned crude (0.2 g) was added to 5 mL of DCM. Intermediate 1 (0.12 g, 0.21 mmol), triethylamine (0.061 g, 0.6 mmol) and HATU (0.15 g, 0.39 mmol) were sequentially added and the mixture was reacted at room temperature for 2 h. To the reaction solution was added 5 mL of water and liquid separation was performed. The aqueous phase was extracted with 6 mL of DCM twice and the organic phases were combined, dried over anhydrous sodium sulfate and concentrated under reduced pressure. The resulting crude was preliminarily separated and purified by chromatographic column on silica gel (dichloromethane:methanol (v/v)=10:1) to afford a crude, which was then passed through Pre-HPLC (instrument and preparative column: using SHIMADZU LC-20AP preparative liquid phase chromatographic instrument, preparative column model: Phenomenex C18). Mobile phase system: acetonitrile/water. Gradient elution method: gradient elution with acetonitrile from 60% to 90% (elution time: 15 min). Lyophilization was performed to afford compound 13 (0.1 g, yield: 15%).

¹H NMR (400 MHz, CDCl₃) δ 8.66 (s, 1H), 8.45-8.32 (m, 1H), 8.06-7.93 (m, 1H), 7.82-7.66 (m, 2H), 7.43-7.11 (m, 13H), 7.03-6.92 (m, 2H), 6.83-6.56 (m, 3H), 6.53-6.32 (m, 1H), 5.15-4.90 (m, 1H), 4.84-4.17 (m, 4H), 4.14-3.86 (m, 2H), 3.68-2.91 (m, 10H), 2.88-2.70 (m, 3H), 2.70-1.75 (m, 22H), 1.75-1.22 (m, 13H), 1.12-0.88 (m, 15H).

LCMS m/z=518.8 [M/3+1]⁺.

Example 14

cis-(2S,4R)-1-((S)-2-(7-(5-((R)-3-((4-(N-(4-(4-((4′-chloro-2′-fluoro-4,4-dimethyl-3,4,5,6-tetrahydro-[1,1′-biphenyl]-2-yl)methyl)piperazin-1-yl)benzoyl) sulfamoyl)-2-((trifluoromethyl)sulfonyl)phenyl)amino)-4-(phenylthio)butyl) hexahydropyrrolo[3,4-c]pyrrol-2(1H)-yl)-7-oxoheptanamido)-3,3-dimethylbutanoyl)-4-hydroxy-N—((S)-1-(4-(4-methylthiazol-5-yl)phenyl)ethyl) pyrrolidine-2-carboxamide (Compound 14)

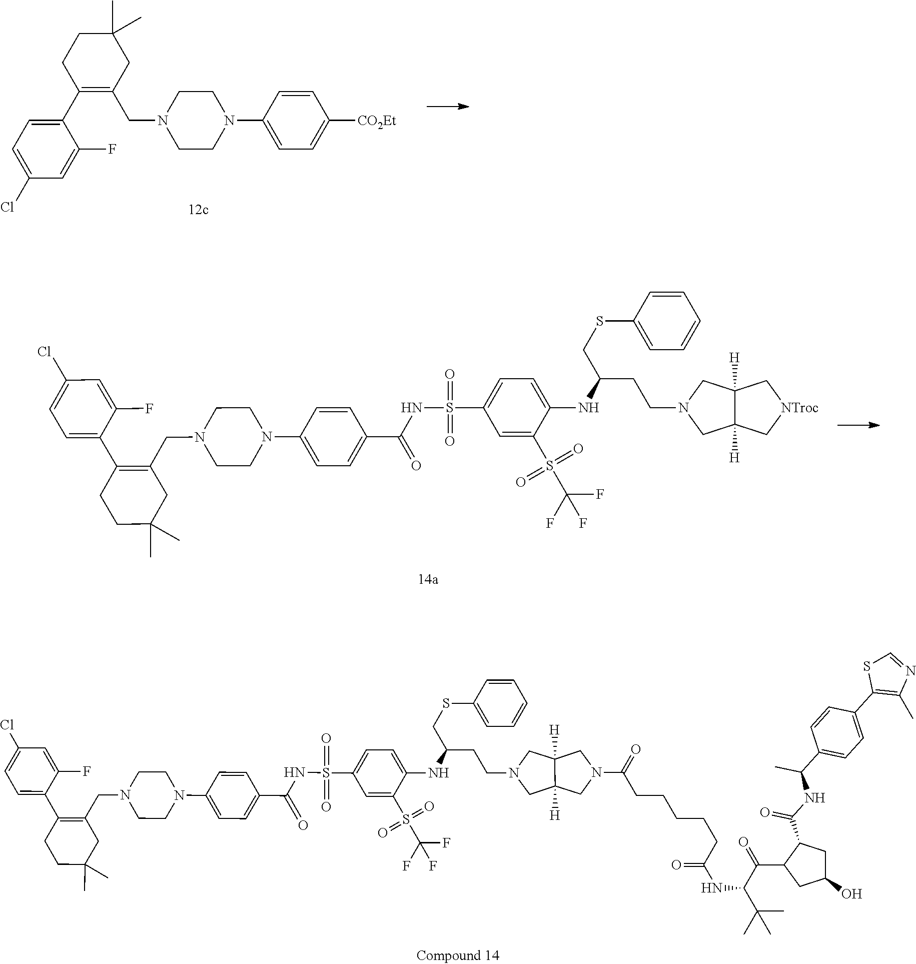

### Step 1: Preparation of 14a

12c (1.4 g, 2.89 mmol) was dissolved in 10 mL of methanol. Water (1 mL) and sodium hydroxide (0.35 g, 8.75 mmol) were added and the mixture was reacted at 80° C. for 10 h. The reaction solution was cooled to room temperature and concentrated under reduced pressure. The residue was dissolved with 10 mL of water and then washed with 20 mL of methyl tert-butyl ether. The aqueous phase was separated, adjusted to pH 6 with 1 mol/L hydrochloric acid, extracted with ethyl acetate (100 mL×2), dried over anhydrous sodium sulfate and concentrated under reduced pressure to afford a crude (0.8 g). The above-mentioned crude (240 mg) was dissolved in 15 mL of DCM. 8f (400 mg, 0.53 mmol), DMAP (130 mg, 1.06 mmol) and EDCI (200 mg, 1.04 mmol) were added and the mixture was reacted at room temperature for 16 h. To the reaction system was slowly added 100 mL of water and the mixture was extracted with DCM (100 mL×2). The organic phase was separated, dried over anhydrous sodium sulfate and concentrated under reduced pressure and the resulting crude was separated and purified by silica gel column chromatography (dichloromethane:methanol (v/v)=0:1-9:1) to afford 14a (500 mg, yield: 48%).

LCMS m/z=597.1 [M/2+1]⁺.

### Step 2: Preparation of Compound 14

14a (500 mg, 0.42 mmol) was dissolved in 40 mL of tetrahydrofuran. Acetic acid (1.2 mL) and zinc powder (1.8 g, 27.75 mmol) were sequentially added and the mixture was reacted at room temperature for 16 h. The reaction system was filtered and to the filtrate were added 10 mL of water and 20 mL of ethyl acetate. The organic phase was separated, dried over anhydrous sodium sulfate and concentrated under reduced pressure. The residue was separated and purified by chromatographic column on silica gel (methanol/dichloromethane/triethylamine (v/v)=1:19:0-10:89:1) to afford a crude (330 mg). The above-mentioned crude (150 mg) was dissolved in 15 mL of DCM. Triethylamine (0.21 mL), intermediate 1 (88 mg, 0.15 mmol) and HATU (86 mg, 0.23 mmol) were sequentially added and the mixture was reacted at room temperature for 2 h. To the reaction system was added 100 mL of water. The mixture was extracted with 50 mL of DCM, dried over anhydrous sodium sulfate and concentrated under reduced pressure. The residue was first separated and purified by silica gel column chromatography (methanol/dichloromethane (v/v)=0:1-1:9) and then the resulting crude was passed through Pre-HPLC (instrument and preparative column: using SHIMADZU LC-20AP preparative liquid phase chromatographic instrument, preparative column model: Phenomenex C18). Mobile phase system: acetonitrile/water. Gradient elution method: gradient elution with acetonitrile from 59% to 89% (elution time: 15 min). Lyophilization was performed to afford compound 14 (35 mg, yield: 12%).

¹H NMR (400 MHz, CDCl₃) δ 8.75-8.60 (m, 1H), 8.55-8.43 (m, 1H), 7.96-7.68 (m, 3H), 7.46-7.15 (m, 10H), 7.15-6.89 (m, 4H), 6.86-6.63 (m, 3H), 6.56-6.30 (m, 1H), 5.15-4.64 (m, 3H), 4.57-4.44 (m, 1H), 4.26-3.85 (m, 2H), 3.80-3.40 (m, 3H), 3.39-2.92 (m, 8H), 2.92-1.95 (m, 28H), 1.85-1.20 (m, 12H), 1.18-0.90 (m, 15H).

LCMS m/z=793.8 [M/2+1]⁺.

Example 15

cis-(2S,4R)-1-((S)-2-(5-(5-((R)-3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)hexahydropyrrolo[3,4-c]pyrrol-2(1H)-yl)-5-oxopentanamido)-3,3-dimethylbutanoyl)-4-hydroxy-N—((S)-1-(4-(4-methylthiazol-5-yl)phenyl)ethyl)pyrrolidine-2-carboxamide (Compound 15)

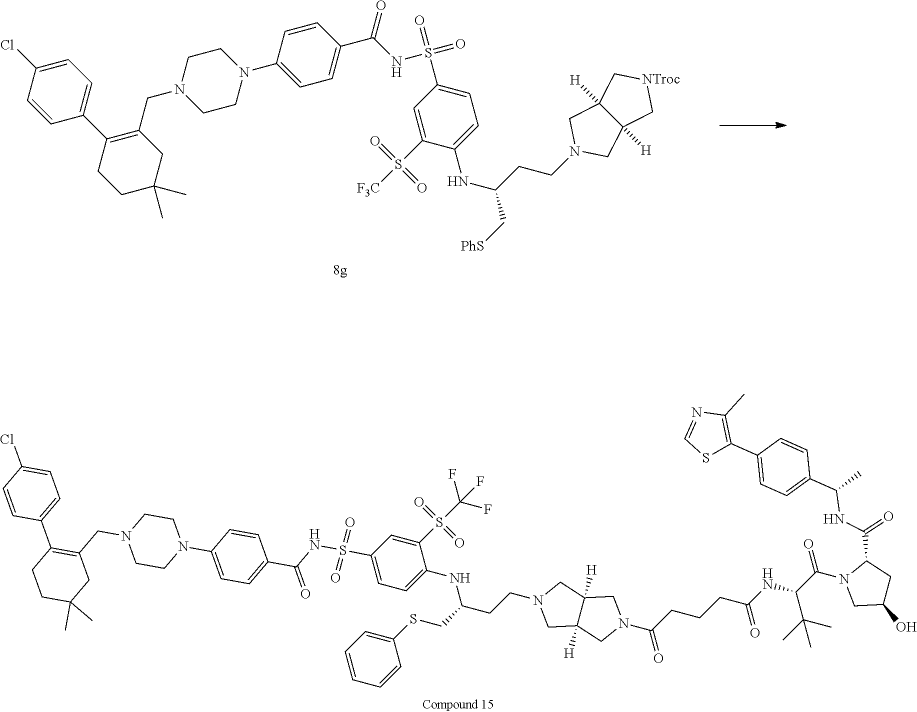

8g (0.44 g, 0.37 mmol) was added to 30 mL of tetrahydrofuran. Zinc powder (1.26 g, 19.27 mmol) and 0.78 mL of acetic acid were sequentially added and the mixture was reacted at room temperature for 5 h. The reaction system was filtered and the filtrate was concentrated under reduced pressure. The residue was then diluted with 100 mL of DCM and then 50 mL of saturated sodium bicarbonate solution was added. The organic phase was separated, dried over anhydrous sodium sulfate and concentrated under reduced pressure to afford a crude (0.11 g). The above-mentioned crude (101 mg) was added to 2 mL of DCM. 5-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentanoic acid (see WO 2020163823 for the synthetic method) (58 mg, 0.104 mmol), triethylamine (0.14 mL, 1.01 mmol) and HATU (57 mg, 0.15 mmol) were sequentially added and the mixture was reacted at room temperature for 2 h. To the reaction system was added 20 mL of water and the mixture was extracted with 20 mL of DCM twice. The organic phase was separated, dried over anhydrous sodium sulfate and concentrated under reduced pressure. Then the resulting crude was passed through Pre-HPLC (instrument and preparative column: using SHIMADZU LC-20AP preparative liquid phase chromatographic instrument, preparative column model: Phenomenex C18). Preparation method: the crude was dissolved with methanol and dimethyl sulfoxide, and filtered with a 0.45 m filter membrane, to prepare into a sample liquid. Mobile phase system: acetonitrile/water (containing 0.05% NH₄HCO₃). Gradient elution method: gradient elution with acetonitrile from 60% to 90% (elution time: 15 min). Lyophilization was performed to afford compound 15 (35 mg, yield: 7%).

¹H NMR (400 MHz, CDCl₃) δ 8.66 (s, 1H), 8.45 (s, 1H), 7.95-7.70 (m, 3H), 7.46-7.14 (m, 12H), 7.10-6.90 (m, 4H), 6.85-6.63 (m, 3H), 5.12-4.92 (m, 1H), 4.82-4.40 (m, 3H), 4.22-3.85 (m, 2H), 3.72-3.38 (m, 3H), 3.35-2.57 (m, 14H), 2.56-1.77 (m, 24H), 1.75-1.55 (m, 1H), 1.53-1.30 (m, 5H), 1.16-0.87 (m, 15H).

LCMS m/z=770.8 [M/2+1]⁺.

Example 16

cis-(2S,4R)-1-((S)-2-(9-(5-((R)-3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)hexahydropyrrolo[3,4-c]pyrrol-2(1H)-yl)-9-oxononanamido)-3,3-dimethylbutanoyl)-4-hydroxy-N—((S)-1-(4-(4-methylthiazol-5-yl)phenyl)ethyl)pyrrolidine-2-carboxamide (Compound 16)

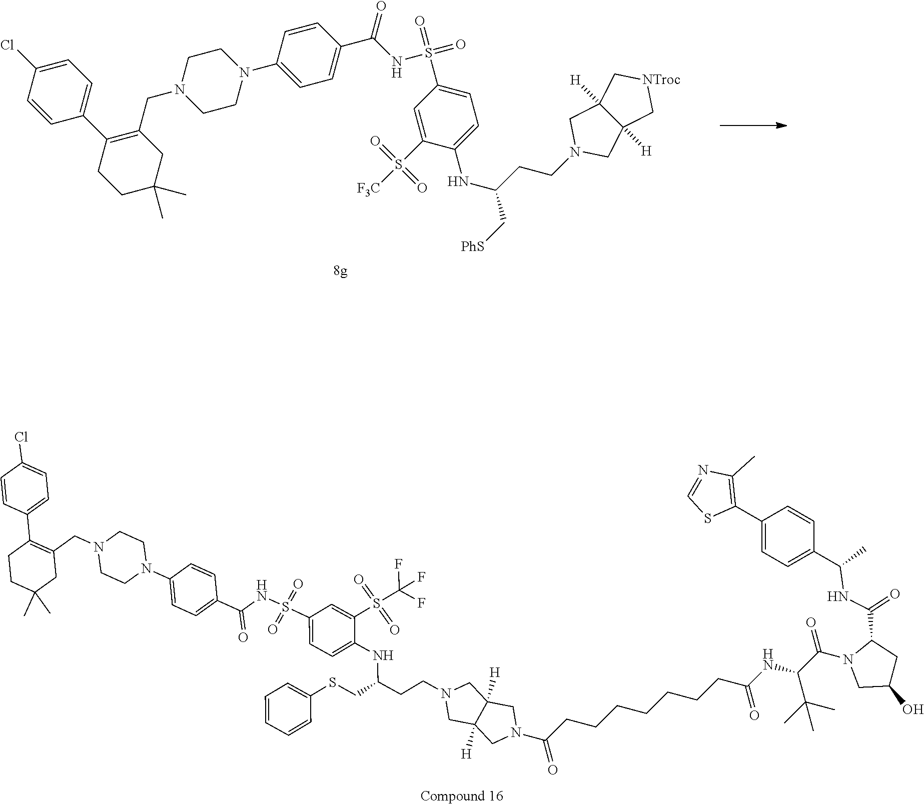

8g (0.44 g, 0.37 mmol) was added to 30 mL of tetrahydrofuran. Zinc powder (1.26 g, 19.27 mmol) and 0.78 mL of acetic acid were sequentially added and the mixture was reacted at room temperature for 5 h. The reaction system was filtered and the filtrate was concentrated under reduced pressure. The residue was then diluted with 100 mL of DCM and then 50 mL of saturated sodium bicarbonate solution was added. The organic phase was separated, dried over anhydrous sodium sulfate and concentrated under reduced pressure to afford a crude (0.11 g). The above-mentioned crude (101 mg) was added to 2 mL of DCM. 9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononanoic acid (see WO 2020163823 for the synthetic method) (61 mg, 0.099 mmol), triethylamine (0.14 mL, 1.01 mmol) and HATU (57 mg, 0.15 mmol) were sequentially added and the mixture was reacted at room temperature for 2 h. To the reaction system was added 30 mL of water and the mixture was extracted with 20 mL of DCM twice. The organic phase was separated, dried over anhydrous sodium sulfate and concentrated under reduced pressure. Then the resulting crude was passed through Pre-HPLC (instrument and preparative column: using SHIMADZU LC-20AP preparative liquid phase chromatographic instrument, preparative column model: Phenomenex C18). Preparation method: the crude was dissolved with methanol and dimethyl sulfoxide, and filtered with a 0.45 m filter membrane, to prepare into a sample liquid. Mobile phase system: acetonitrile/water (containing 0.05% NH₄HCO₃). Gradient elution method: gradient elution with acetonitrile from 60% to 90% (elution time: 15 min). Lyophilization was performed to afford compound 16 (85 mg, yield: 16%).

¹H NMR (400 MHz, CDCl₃) δ 8.66 (s, 1H), 8.52-8.34 (m, 1H), 8.07-7.87 (m, 1H), 7.83-7.67 (m, 2H), 7.50-7.16 (m, 12H), 7.11-6.90 (m, 3H), 6.85-6.55 (m, 3H), 6.40-6.25 (m, 1H), 5.18-5.00 (m, 1H), 4.80-4.58 (m, 2H), 4.56-4.45 (m, 1H), 4.18-4.05 (m, 1H), 4.00-3.85 (m, 1H), 3.70-3.42 (m, 3H), 3.35-2.95 (m, 8H), 2.94-2.64 (m, 5H), 2.63-1.95 (m, 23H), 1.80-1.36 (m, 10H), 1.35-1.15 (m, 6H), 1.10-0.90 (m, 15H).

LCMS m/z=798.2 [M/2+1]⁺.

Example 17

cis-(2S,4R)-1-((S)-2-(8-(5-((R)-3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)hexahydropyrrolo[3,4-c]pyrrol-2(1H)-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (Compound 17)

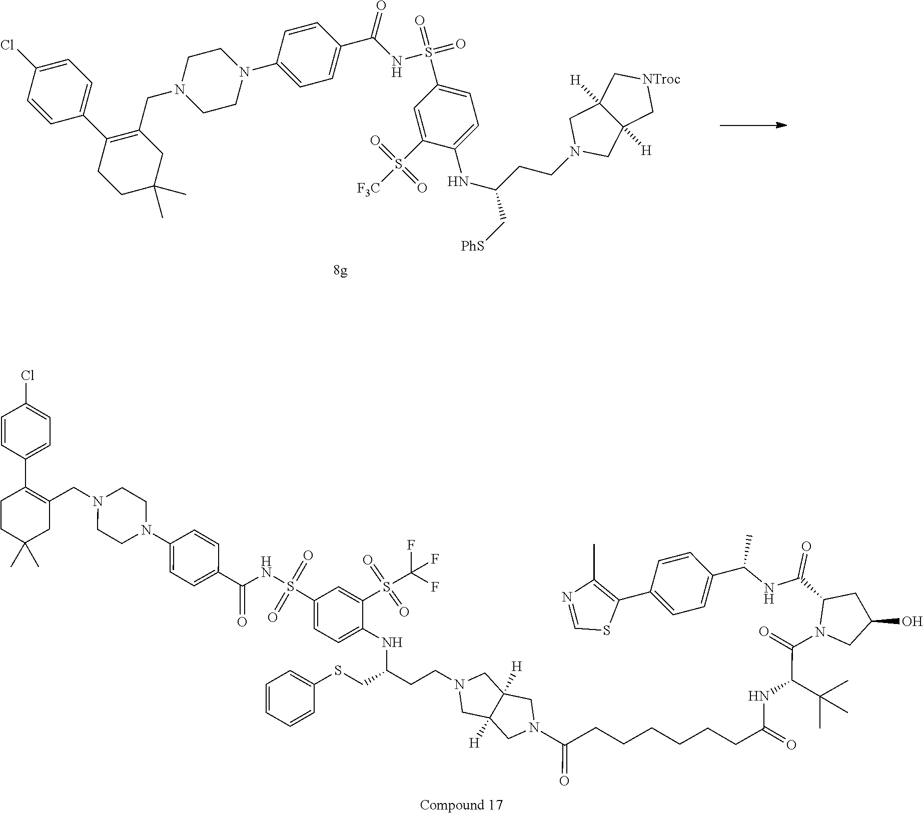

8g (0.30 g, 0.25 mmol) was added to 300 mL of tetrahydrofuran. Zinc powder (1.36 g, 20.8 mmol) and ammonium chloride (0.41 g, 7.66 mmol) were sequentially added and the mixture was reacted at room temperature for 16 h. The reaction system was filtered and to the filtrate were added 10 mL of water and 20 mL of ethyl acetate, followed by extraction. The organic phase was dried over anhydrous sodium sulfate and concentrated under reduced pressure. The resulting crude was separated and purified by chromatographic column on silica gel (dichloromethane:methanol:triethylamine (v/v)=95:5:0-89:10:1) to afford a crude (0.26 g). The above-mentioned crude (120 mg) was added to 15 mL of DCM. Intermediate 3 (75 mg, 0.12 mmol), triethylamine (0.17 mL, 1.2 mmol) and HATU (68 mg, 0.18 mmol) were sequentially added and the mixture was reacted at room temperature for 2 h. To the reaction system was added 100 mL of water. The mixture was extracted with 200 mL of DCM, dried over anhydrous sodium sulfate and concentrated under reduced pressure. The crude was separated and purified by silica gel column chromatography (methanol/dichloromethane (v/v)=0:1-1:9) and then the resulting crude was passed through Pre-HPLC (instrument and preparative column: using SHIMADZU LC-20AP preparative liquid phase chromatographic instrument, preparative column model: Phenomenex C18). Preparation method: the crude was dissolved with methanol and dimethyl sulfoxide, and filtered with a 0.45 m filter membrane, to prepare into a sample liquid. Mobile phase system: acetonitrile/water. Gradient elution method: gradient elution with acetonitrile from 59% to 89% (elution time: 15 min). Lyophilization was performed to afford compound 17 (90 mg, yield: 49%).

¹H NMR (400 MHz, CDCl₃) δ 8.66 (s, 1H), 8.52-8.39 (m, 1H), 8.05-7.86 (m, 1H), 7.85-7.70 (m, 2H), 7.58-7.14 (m, 12H), 7.07-6.92 (m, 3H), 6.86-6.60 (m, 3H), 6.54-6.29 (m, 1H), 5.15-4.92 (m, 1H), 4.84-4.60 (m, 2H), 4.58-4.42 (m, 1H), 4.19-3.85 (m, 2H), 3.68-3.40 (m, 3H), 3.35-2.94 (m, 8H), 2.94-1.95 (m, 28H), 1.78-1.15 (m, 14H), 1.15-0.95 (m, 15H).

LCMS m/z=791.8 [M/2+1]⁺.

Example 18

cis-(2S,4R)-1-((S)-2-(7-(5-((R)-3-((4-(N-(4-(4-((6-(4-chlorophenyl)spiro[2.5]oct-5-en-5-yl)methyl)piperazin-1-yl)benzoyl)sulfamoyl)-2-((trifluoromethyl)sulfonyl)phenyl)amino)-4-(phenylthio)butyl)hexahydropyrrolo[3,4-c]pyrrol-2(1H)-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (compound 18)

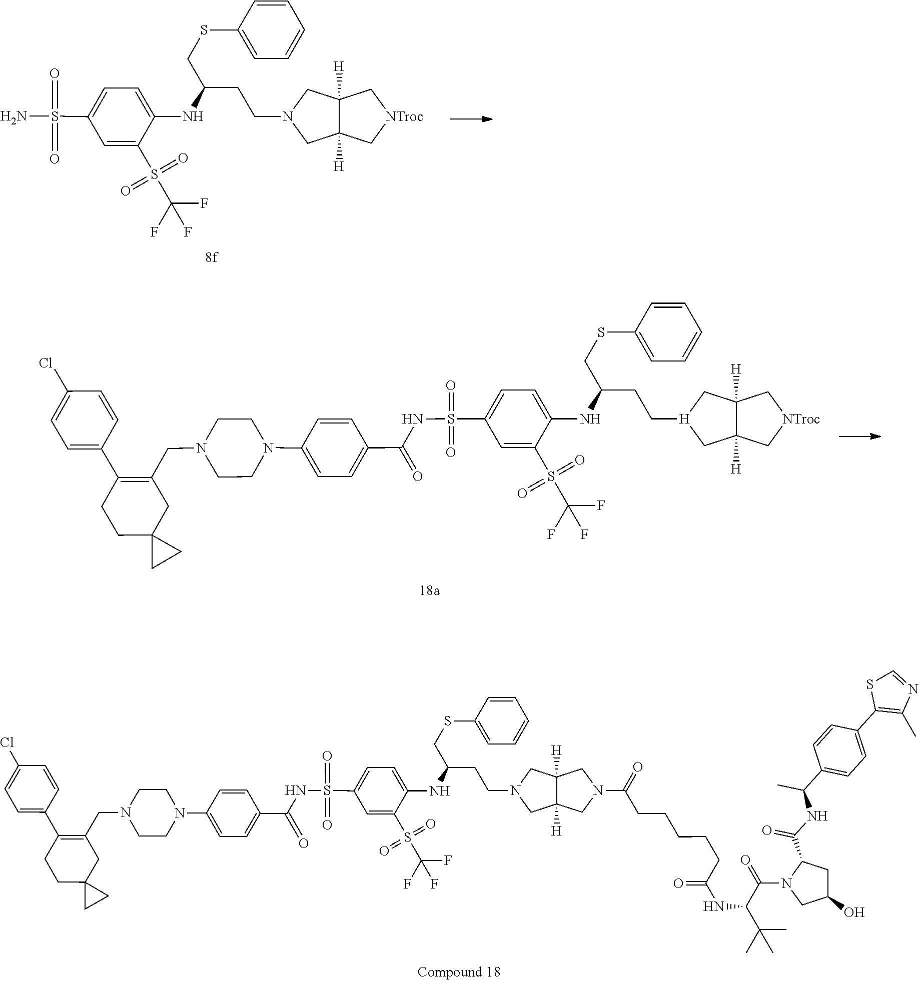

Step 1: Preparation of 18a

11a (170 mg, 0.39 mmol) was dissolved in 15 mL of DCM. 8f (300 mg, 0.40 mmol), DMAP (100 mg, 0.82 mmol) and EDCI (150 mg, 0.78 mmol) were sequentially added and the mixture was reacted at room temperature for 16 h. To the reaction system was slowly added 100 mL of water and the mixture was extracted with 100 mL of DCM. The organic phase was dried over anhydrous sodium sulfate and concentrated under reduced pressure and the resulting crude was separated and purified by silica gel column chromatography (dichloromethane/methanol (v/v)=0:1-9:1) to afford 18a (300 mg, yield: 66%).

LCMS m/z=587.2 [M/2+1]⁺.

### Step 2: Preparation of Compound 18

18a (300 mg, 0.26 mmol) was dissolved in 5 mL of tetrahydrofuran and 15 mL of methanol. Ammonium chloride (0.41 g, 7.66 mmol) and zinc powder (1.36 g, 20.9 mmol) were sequentially added and the mixture was reacted at room temperature for 16 h. The reaction system was filtered and to the filtrate were added 10 mL of water and 20 mL of ethyl acetate, followed by extraction. The organic phase was dried over anhydrous sodium sulfate and concentrated under reduced pressure. The resulting crude was separated and purified by chromatographic column on silica gel (dichloromethane:methanol:triethylamine (v/v)=95:5:0-89:10:1) to afford a crude (220 mg). The above-mentioned crude (120 mg) was dissolved in 15 mL of DCM. Triethylamine (0.17 mL), intermediate 1 (70 mg, 0.12 mmol) and HATU (68 mg, 0.18 mmol) were sequentially added and the mixture was reacted at room temperature for 2 h. To the reaction system was added 100 mL of water. The mixture was extracted with 50 mL of DCM, dried over anhydrous sodium sulfate and concentrated under reduced pressure. The crude was separated and purified by silica gel column chromatography (methanol/dichloromethane (v/v)=0:1-1:9) and then the resulting crude was passed through Pre-HPLC (instrument and preparative column: using SHIMADZU LC-20AP preparative liquid phase chromatographic instrument, preparative column model: Phenomenex C18). Preparation method: the crude was dissolved with methanol and dimethyl sulfoxide, and filtered with a 0.45 m filter membrane, to prepare into a sample liquid. Mobile phase system: acetonitrile/water (containing 0.05% NH₄HCO₃). Gradient elution method: gradient elution with acetonitrile from 40% to 70% (elution time: 15 min). Lyophilization was performed to afford compound 18 (35 mg, yield: 16%).

¹H NMR (400 MHz, CDCl₃) δ 8.66 (s, 1H), 8.42-8.32 (m, 1H), 8.15-7.90 (m, 1H), 7.80-7.60 (m, 2H), 7.44-6.96 (m, 15H), 6.83-6.47 (m, 3H), 6.43-6.16 (m, 1H), 5.15-5.02 (m, 1H), 4.85-3.83 (m, 6H), 3.65-2.68 (m, 15H), 2.68-1.17 (m, 35H), 1.04 (s, 9H), 0.36 (s, 4H).

LCMS m/z=783.9 [M/2+1]⁺.

Example 19

(2S,4R)-1-((S)-2-(7-((1R,4R)-5-((R)-3-((4-(N-(4-(4-((6-(4-chlorophenyl)spiro[2.5]oct-5-en-5-yl)methyl)piperazin-1-yl)benzoyl)sulfamoyl)-2-((trifluoromethyl)sulfonyl)phenyl)amino)-4-(phenylthio)butyl)-2,5-diazabicyclo[2.2.1]heptan-2-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 19)

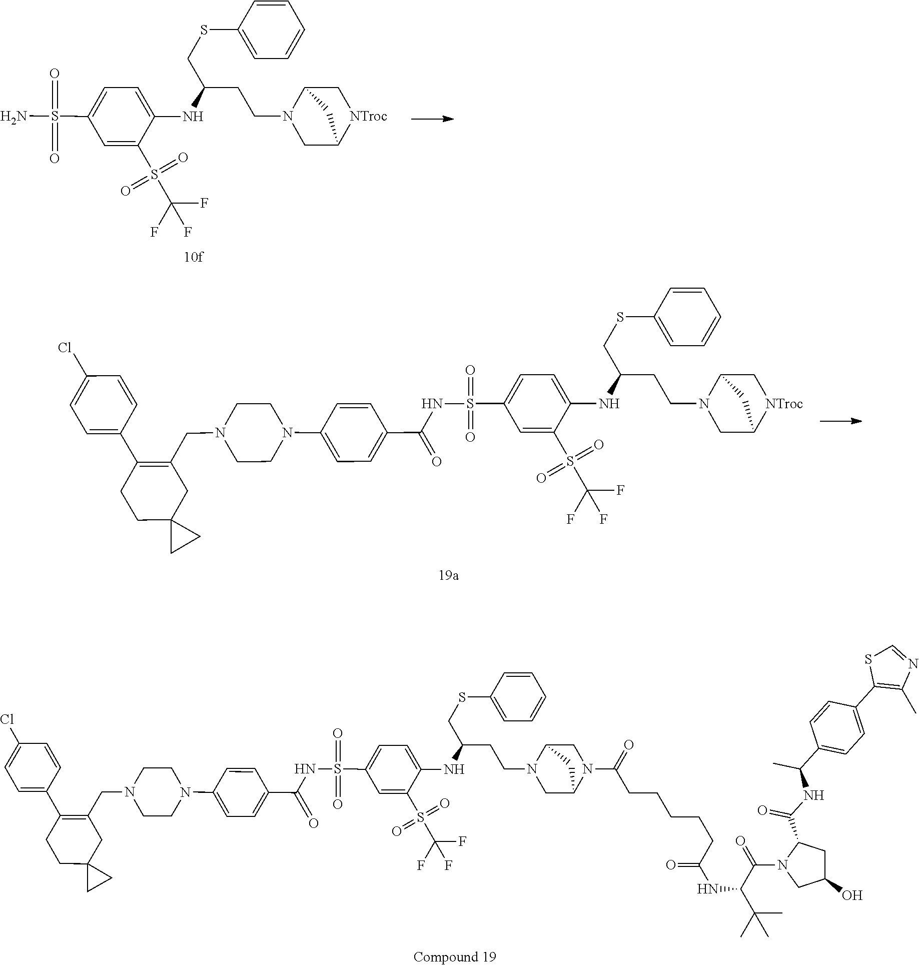

### Step 1: Preparation of 19a

11a (150 mg, 0.34 mmol) was dissolved in 10 mL of DCM. 10f (250 mg, 0.34 mmol), DMAP (83 mg, 0.68 mmol) and EDCI (130 mg, 0.68 mmol) were sequentially added and the mixture was reacted at room temperature for 16 h. To the reaction system was slowly added 100 mL of water and the mixture was extracted with 100 mL of dichloromethane. The organic phase was dried over anhydrous sodium sulfate and concentrated under reduced pressure and the resulting crude was separated and purified by silica gel column chromatography (dichloromethane/methanol (v/v)=0:1-9:1) to afford 19a (220 mg, yield: 56%).

LCMS m/z=580.0 [M/2+1]⁺.

### Step 2: Preparation of Compound 19

19a (220 mg, 0.19 mmol) was dissolved in 3 mL of tetrahydrofuran and 12 mL of methanol. Ammonium chloride (0.32 g, 5.98 mmol) and zinc powder (1.05 g, 16 mmol) were sequentially added and the mixture was reacted at room temperature for 16 h. The reaction system was filtered and to the filtrate were added 10 mL of water and 20 mL of ethyl acetate for extraction, followed by liquid separation. The organic phase was dried over anhydrous sodium sulfate and concentrated under reduced pressure and the resulting crude was separated and purified by chromatographic column on silica gel (dichloromethane:methanol:triethylamine (v/v/v)=95:5:0-89:10:1) to afford a crude (0.16 g). The above-mentioned crude (120 mg) was dissolved in 15 mL of DCM. Triethylamine (0.17 mL), intermediate 1 (70 mg, 0.12 mmol) and HATU (68 mg, 0.18 mmol were sequentially added and the mixture was reacted at room temperature for 2 h. To the reaction system was added 100 mL of water. The mixture was extracted with 50 mL of dichloromethane, dried over anhydrous sodium sulfate and concentrated under reduced pressure. The crude was separated and purified by silica gel column chromatography (methanol/dichloromethane (v/v)=0:1-1:9) and then the resulting crude was passed through Pre-HPLC (instrument and preparative column: using SHIMADZU LC-20AP preparative liquid phase chromatographic instrument, preparative column model: Phenomenex C18). Preparation method: the crude was dissolved with methanol and dimethyl sulfoxide, and filtered with a 0.45 m filter membrane, to prepare into a sample liquid. Mobile phase system: acetonitrile/water (containing 0.05% NH₄HCO₃). Gradient elution method: gradient elution with acetonitrile from 50% to 80% (elution time: 15 min). Lyophilization was performed to afford compound 19 (40 mg, yield: 18%).

¹H NMR (400 MHz, CDCl₃) δ 8.75-8.61 (m, 1H), 8.54-8.44 (m, 1H), 7.97-7.70 (m, 3H), 7.48-6.87 (m, 15H), 6.87-6.64 (m, 3H), 6.55-6.32 (m, 1H), 5.17-4.89 (m, 1H), 4.87-4.62 (m, 2H), 4.59-4.45 (m, 1H), 4.25-3.80 (m, 3H), 3.79-2.58 (m, 18H), 2.58-1.95 (m, 21H), 1.63-1.20 (m, 9H), 1.04 (s, 9H), 0.37 (s, 4H).

LCMS m/z=776.5 [M/2+1]⁺.

Example 20

(2S,4R)-1-((S)-2-(7-((1S,4S)-5-((R)-3-((4-(N-(4-(4-((4′-chloro-2′-fluoro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)-2,5-diazabicyclo[2.2.1]heptan-2-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 20)

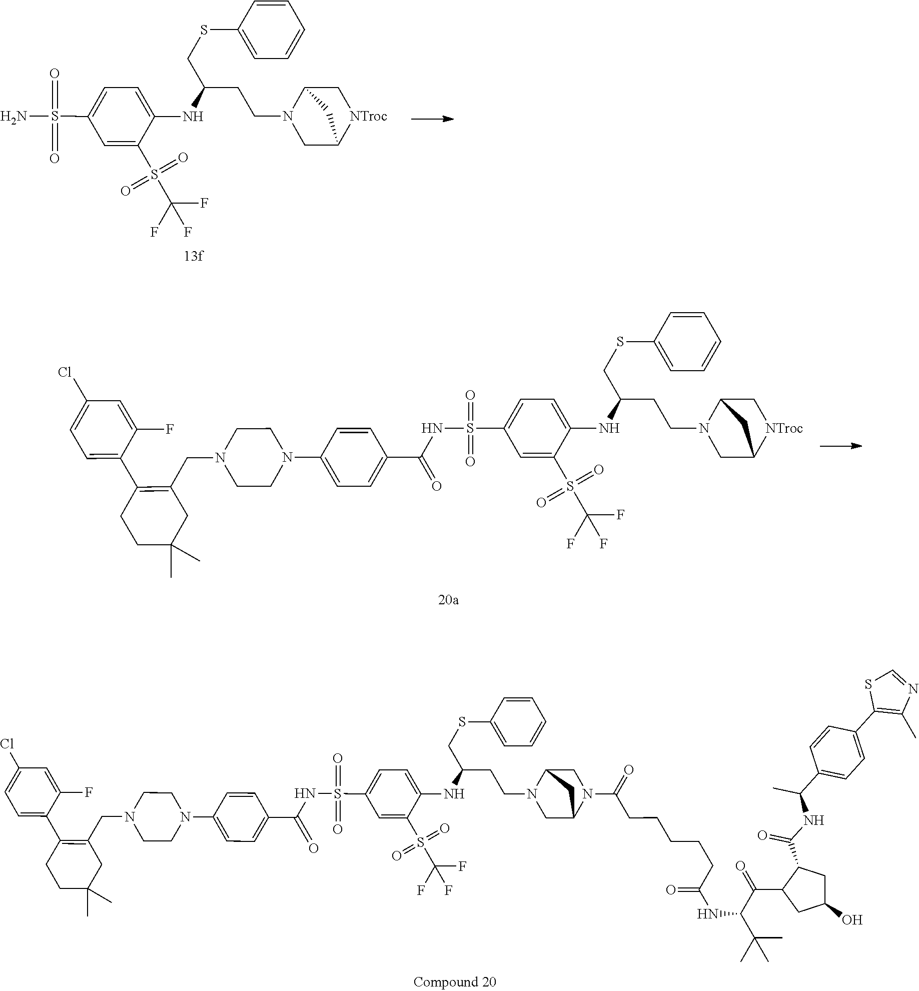

### Step 1: Preparation of 20a

12c (1.4 g, 2.89 mmol) was dissolved in 10 mL of methanol. Water (1 mL) and sodium hydroxide (0.35 g, 8.75 mmol) were added and the mixture was reacted at 80° C. for 10 h. The reaction solution was cooled to room temperature and concentrated under reduced pressure. The residue was dissolved with 10 mL of water and then washed with 20 mL of methyl tert-butyl ether. The aqueous phase was separated, adjusted to pH 6 with 1 mol/L hydrochloric acid, extracted with ethyl acetate (100 mL×2), dried over anhydrous sodium sulfate and concentrated under reduced pressure to afford a crude (0.8 g). The above-mentioned crude (250 mg) was dissolved in 10 mL of DCM. 13f (410 mg, 0.55 mmol), DMAP (130 mg, 1.07 mmol) and EDCI (210 mg, 1.10 mmol) were added and the mixture was reacted at room temperature for 16 h. To the reaction system was slowly added 100 mL of water and the mixture was extracted with 50 mL of DCM. The organic phase was dried over anhydrous sodium sulfate and concentrated under reduced pressure and the resulting crude was separated and purified by silica gel column chromatography (dichloromethane/methanol (v/v)=1:0-9:1) to afford 20a (310 mg, yield: 48%).

LCMS m/z=590.2 [M/2+1]⁺.

### Step 2: Preparation of Compound 20

20a (300 mg, 0.25 mmol) was dissolved in 3 mL of tetrahydrofuran and 12 mL of methanol. Ammonium chloride (0.4 g, 7.5 mmol) and zinc powder (1.31 g, 20.15 mmol) were added and the mixture was reacted at room temperature for 16 h. The reaction system was filtered and to the filtrate were added 10 mL of water and 20 mL of ethyl acetate, followed by extraction. The organic phase was dried over anhydrous sodium sulfate and concentrated under reduced pressure. The resulting crude was separated and purified by chromatographic column on silica gel (dichloromethane:methanol:triethylamine (v/v/v)=95:5:0-89:10:1) to afford a crude (0.22 g). The above-mentioned crude (27 mg) was dissolved in 5 mL of DCM. Triethylamine (0.037 mL), intermediate 1 (16 mg, 0.027 mmol) and HATU (15 mg, 0.04 mmol) were sequentially added and the mixture was reacted at room temperature for 2 h. To the reaction system was added 100 mL of water. The mixture was extracted with 20 mL of DCM, dried over anhydrous sodium sulfate and concentrated under reduced pressure. The crude was separated and purified by silica gel column chromatography (methanol/dichloromethane (v/v)=0:1-1:9) and then the resulting crude was passed through Pre-HPLC (instrument and preparative column: using SHIMADZU LC-20AP preparative liquid phase chromatographic instrument, preparative column model: Phenomenex C18). Preparation method: the crude was dissolved with methanol and dimethyl sulfoxide, and filtered with a 0.45 m filter membrane, to prepare into a sample liquid. Mobile phase system: acetonitrile/water (containing 0.05% NH₄HCO₃). Gradient elution method: gradient elution with acetonitrile from 40% to 70% (elution time: 15 min). Lyophilization was performed to afford compound 20 (18 mg, yield: 37%).

¹H NMR (400 MHz, CDCl₃) δ 8.72-8.60 (m, 1H), 8.50-8.34 (m, 1H), 8.08-7.90 (m, 1H), 7.80-7.64 (m, 2H), 7.48-6.88 (m, 14H), 6.85-6.55 (m, 3H), 6.49-6.25 (m, 1H), 5.15-4.90 (m, 1H), 4.85-3.85 (m, 6H), 3.68-2.90 (m, 10H), 2.86-1.94 (m, 22H), 1.90-1.18 (m, 16H), 1.12-0.88 (m, 15H).

LCMS m/z=524.8 [M/3+1]⁺.

Example 21

cis-(2S,4R)-1-((S)-2-(7-(5-((R)-3-((4-(N-(4-(4-((6-(4-chloro-2-fluorophenyl)spiro[2.5]oct-5-en-5-yl)methyl)piperazin-1-yl)benzoyl)sulfamoyl)-2-((trifluoromethyl)sulfonyl)phenyl)amino)-4-(phenylthio)butyl)hexahydropyrrolo[3,4-c]pyrrol-2(1H)-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 21)

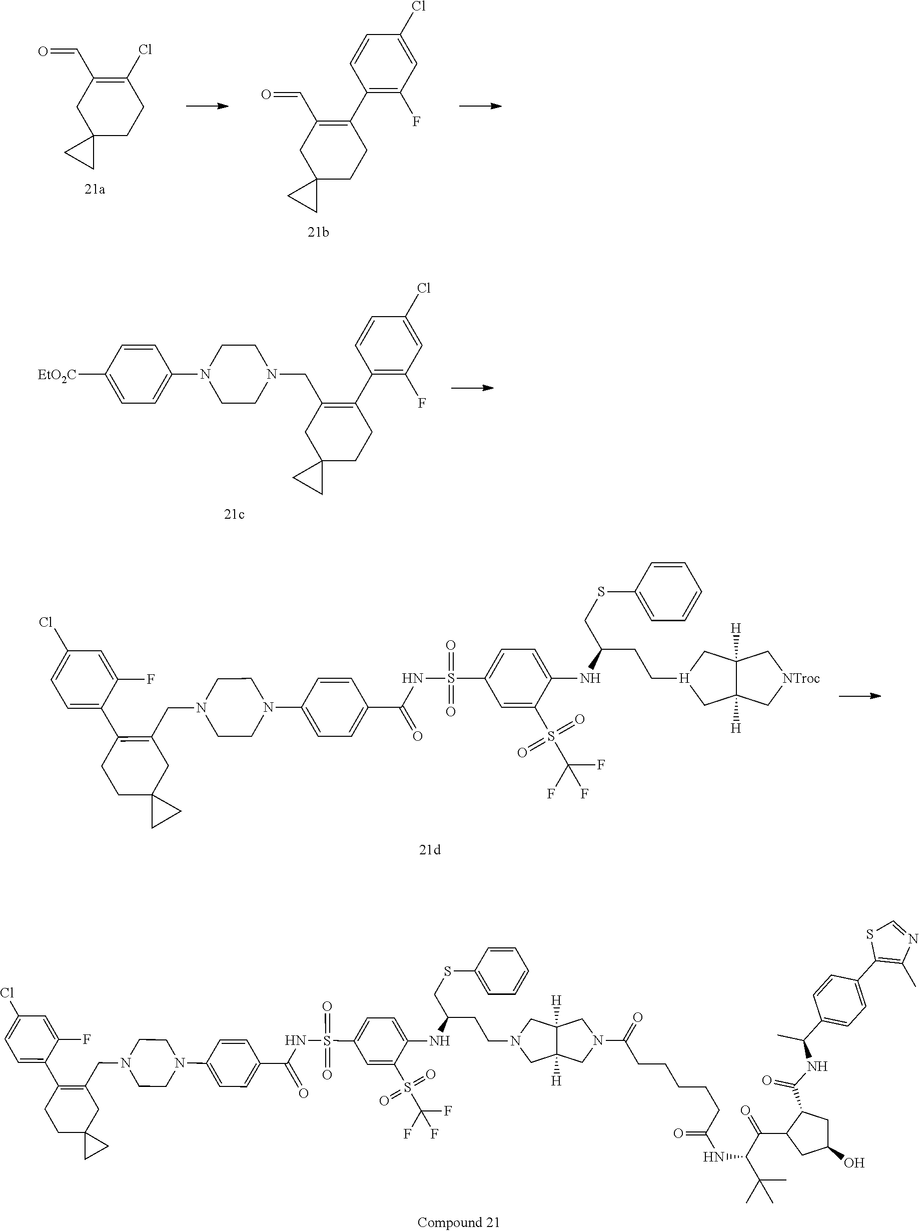

### Step 1: Preparation of 21b

21a (see WO 2013185202 for the synthetic method) (1.00 g, 5.86 mmol) and (4-chloro-2-fluorophenyl)boronic acid (1.02 g, 5.85 mmol) were dissolved in 30 mL of dioxane and 3 mL of water. After nitrogen replacement was carried out three times, potassium acetate (1.73 g, 17.63 mmol) and Pd(dppf)Cl₂ (0.13 g, 0.18 mmol) were added under nitrogen protection and the mixture was reacted at 90° C. for 7 h. The reaction solution was cooled to room temperature and to the reaction solution was slowly added 40 mL of water. The mixture was extracted with 40 mL of ethyl acetate, dried over anhydrous sodium sulfate and concentrated under reduced pressure and the resulting crude was separated and purified by silica gel column chromatography (ethyl acetate/petroleum ether (v/v)=0:1-1:10) to afford 21b (0.78 g, yield: 50%).

### Step 2: Preparation of 21c

21b (780 mg, 2.95 mmol) was dissolved in 35 mL of tetrahydrofuran. Ethyl 4-(piperazin-1-yl)benzoate (1.11 g, 4.74 mmol), 2 mL of acetic acid and sodium triacetoxyborohydride (2.01 g, 9.48 mmol) were sequentially added and the mixture was reacted at room temperature for 16 h. To the reaction solution was slowly added 50 mL of saturated sodium bicarbonate solution. The resulting mixture was extracted with 60 mL of ethyl acetate twice and the organic phase was washed with 50 mL of saturated sodium chloride, dried over anhydrous sodium sulfate and concentrated under reduced pressure. The resulting crude was separated and purified by silica gel column chromatography (ethyl acetate/petroleum ether (v/v)=0:1-1:1) to afford 21c (1.27 g, yield: 89%).

LCMS m/z=483.3 [M+1]⁺.

### Step 3: Preparation of 21d

Sodium hydroxide (0.26 g, 6.5 mmol) was dissolved in 5 mL of methanol and 3 mL of water. A solution of 21c (1.27 g, 2.63 mmol) in 10 mL of tetrahydrofuran was added and the resulting mixture was reacted at 80° C. for 6 h. The reaction solution was cooled to room temperature and concentrated under reduced pressure. The residue was adjusted to pH 5 with 1 mol/L hydrochloric acid and extracted with ethyl acetate (15 mL×3). The organic phase was washed with 20 mL of saturated sodium chloride solution, dried over anhydrous sodium sulfate and concentrated under reduced pressure to afford a crude (0.96 g). The above-mentioned crude (170 mg) was dissolved in 15 mL of DCM. 8f (300 mg, 0.40 mmol), DMAP (100 mg, 0.82 mmol) and EDCI (150 mg, 0.78 mmol) were added and the mixture was reacted at room temperature for 16 h. To the reaction system was slowly added 100 mL of water and the mixture was extracted with 100 mL of DCM. The organic phase was dried over anhydrous sodium sulfate and concentrated under reduced pressure and the resulting crude was separated and purified by silica gel column chromatography (dichloromethane/methanol (v/v)=0:1-9:1) to afford 21d (350 mg, yield: 73%).

LCMS m/z=596.1 [M/2+1]⁺.

### Step 4: Preparation of Compound 21

21d (350 mg, 0.294 mmol) was dissolved in 5 mL of tetrahydrofuran and 15 mL of methanol. Ammonium chloride (0.48 g, 8.97 mmol) and zinc powder (1.57 g, 24.15 mmol) were added and the mixture was reacted at room temperature for 16 h. The reaction system was filtered and to the filtrate were added 10 mL of water and 20 mL of ethyl acetate, followed by extraction. The organic phase was separated, dried over anhydrous sodium sulfate and concentrated under reduced pressure. The residue was separated and purified by chromatographic column on silica gel (methanol/dichloromethane/triethylamine (v/v/v)=1:19:0-10:89:1) to afford a crude (230 mg). The above-mentioned crude (100 mg) was dissolved in 15 mL of DCM. Triethylamine (0.14 mL), intermediate 1 (60 mg, 0.10 mmol) and HATU (57 mg, 0.15 mmol) were sequentially added and the mixture was reacted at room temperature for 2 h. To the reaction system was added 100 mL of water. The mixture was extracted with 50 mL of DCM, dried over anhydrous sodium sulfate and concentrated under reduced pressure. The residue was first separated and purified by silica gel column chromatography (methanol/dichloromethane (v/v)=0:1-1:9) and subsequently, the resulting crude was passed through Pre-HPLC (instrument and preparative column: using SHIMADZU LC-20AP preparative liquid phase chromatographic instrument, preparative column model: Phenomenex C18). Mobile phase system: acetonitrile/water (containing 0.05% NH₄HCO₃). Gradient elution method: gradient elution with acetonitrile from 40% to 70% (elution time: 15 min). Lyophilization was performed to afford compound 21 (35 mg, yield: 17%).

¹H NMR (400 MHz, CDCl₃) δ 8.73-8.60 (m, 1H), 8.55-8.45 (m, 1H), 7.95-7.70 (m, 3H), 7.44-7.14 (m, 10H), 7.13-6.90 (m, 4H), 6.86-6.62 (m, 3H), 6.52-6.32 (m, 1H), 5.15-4.88 (m, 1H), 4.87-4.63 (m, 2H), 4.57-4.43 (m, 1H), 4.27-3.82 (m, 2H), 3.80-3.36 (m, 3H), 3.35-2.93 (m, 8H), 2.91-1.95 (m, 25H), 1.68-1.39 (m, 11H), 1.38-1.20 (m, 4H), 1.15-0.95 (m, 9H), 0.37 (s, 4H).

LCMS m/z=528.9 [M/3+1]⁺.

Example 22

cis-(2S,4R)-1-((S)-2-(8-(5-((R)-3-((4-(N-(4-(4-((6-(4-chlorophenyl)spiro[2.5]oct-5-en-5-yl)methyl)piperazin-1-yl)benzoyl)sulfamoyl)-2-((trifluoromethyl)sulfonyl)phenyl)amino)-4-(phenylthio)butyl)hexahydropyrrolo[3,4-c]pyrrol-2(1H)-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (Compound 22)

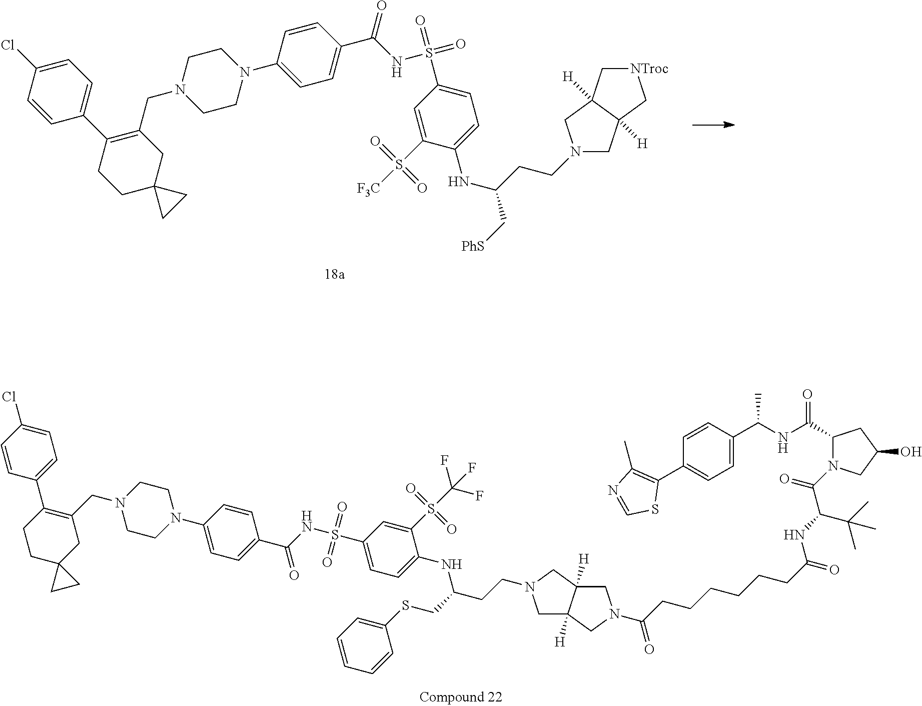

18a (300 mg, 0.26 mmol) was dissolved in 5 mL of tetrahydrofuran and 15 mL of methanol. Ammonium chloride (0.41 g, 7.66 mmol) and zinc powder (1.36 g, 20.9 mmol) were sequentially added and the mixture was reacted at room temperature for 16 h. The reaction system was filtered and to the filtrate were added 10 mL of water and 20 mL of ethyl acetate, followed by extraction. The organic phase was dried over anhydrous sodium sulfate and concentrated under reduced pressure. The resulting crude was separated and purified by chromatographic column on silica gel (dichloromethane:methanol:triethylamine (v/v/v)=95:5:0-89:10:1) to afford a crude (0.22 g). The above-mentioned crude (120 mg) was added to 15 mL of DCM. Intermediate 3 (70 mg, 0.11 mmol), triethylamine (0.17 mL, 1.2 mmol) and HATU (68 mg, 0.18 mmol) were sequentially added and the mixture was reacted at room temperature for 2 h. To the reaction system was added 100 mL of water. The mixture was extracted with 200 mL of DCM, dried over anhydrous sodium sulfate and concentrated under reduced pressure. The crude was separated and purified by silica gel column chromatography (methanol/dichloromethane (v/v)=0:1-1:9) and then the resulting crude was passed through Pre-HPLC (instrument and preparative column: using SHIMADZU LC-20AP preparative liquid phase chromatographic instrument, preparative column model: Phenomenex C18). Preparation method: the crude was dissolved with methanol and dimethyl sulfoxide, and filtered with a 0.45 m filter membrane, to prepare into a sample liquid. Mobile phase system: acetonitrile/water (containing 10 mmol/L NH₄HCO₃). Gradient elution method: gradient elution with acetonitrile from 35% to 65% (elution time: 15 min). Lyophilization was performed to afford compound 22 (70 mg, yield: 31%).

¹H NMR (400 MHz, CDCl₃) δ 8.67 (s, 1H), 8.54-8.38 (m, 1H), 8.04-7.70 (m, 3H), 7.57-7.14 (m, 12H), 7.10-6.92 (m, 3H), 6.90-6.59 (m, 3H), 6.52-6.18 (m, 1H), 5.16-4.92 (m, 1H), 4.85-4.62 (m, 2H), 4.59-4.42 (m, 1H), 4.20-3.82 (m, 2H), 3.68-3.40 (m, 3H), 3.38-2.93 (m, 8H), 2.93-1.95 (m, 26H), 1.80-1.42 (m, 11H), 1.41-1.16 (m, 5H), 1.12-0.94 (m, 9H), 0.36 (s, 4H).

LCMS m/z=790.5 [M/2+1]⁺.

Example 23

cis-(2S,4R)-1-((S)-2-(8-(5-((R)-3-((4-(N-(4-(4-((6-(4-chloro-2-fluorophenyl)spiro[2.5]oct-5-en-5-yl)methyl)piperazin-1-yl)benzoyl)sulfamoyl)-2-((trifluoromethyl)sulfonyl)phenyl)amino)-4-(phenylthio)butyl)hexahydropyrrolo[3,4-c]pyrrol-2(1H)-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (Compound 23)

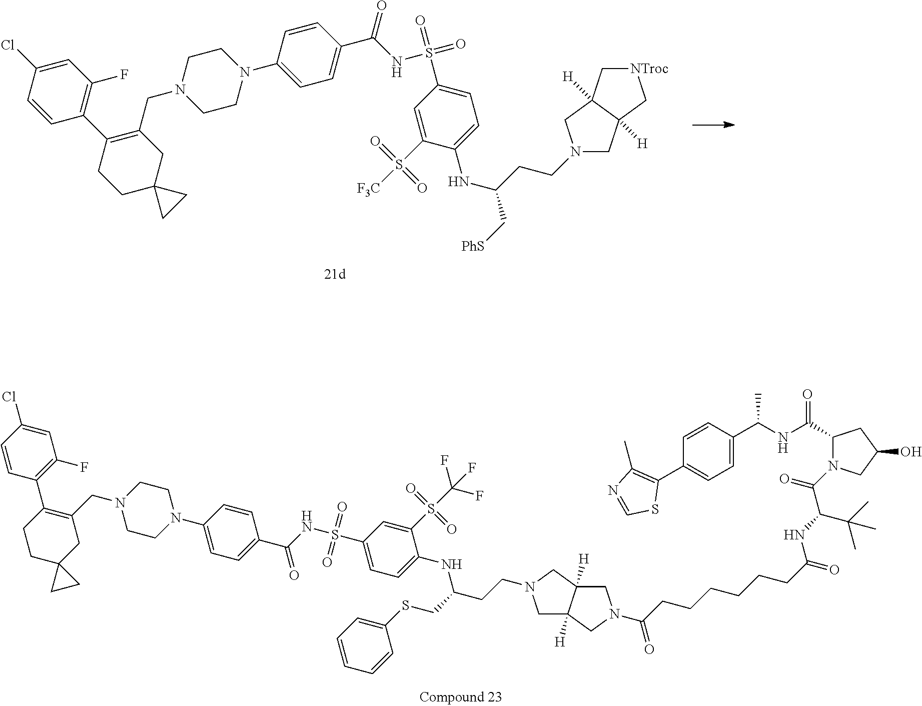

21d (350 mg, 0.294 mmol) was dissolved in 5 mL of tetrahydrofuran and 15 mL of methanol. Ammonium chloride (0.48 g, 8.97 mmol) and zinc powder (1.57 g, 24.15 mmol) were added and the mixture was reacted at room temperature for 16 h. The reaction system was filtered and to the filtrate were added 10 mL of water and 20 mL of ethyl acetate, followed by extraction. The organic phase was dried over anhydrous sodium sulfate and concentrated under reduced pressure. The residue was separated and purified by chromatographic column on silica gel (methanol/dichloromethane/triethylamine (v/v)=1:19:0-10:89:1) to afford a crude (230 mg). The above-mentioned crude (120 mg) was added to 15 mL of DCM. Intermediate 3 (70 mg, 0.11 mmol), triethylamine (0.17 mL, 1.2 mmol) and HATU (68 mg, 0.18 mmol) were sequentially added and the mixture was reacted at room temperature for 2 h. To the reaction system was added 100 mL of water. The mixture was extracted with 200 mL of DCM, dried over anhydrous sodium sulfate and concentrated under reduced pressure. The crude was separated and purified by silica gel column chromatography (methanol/dichloromethane (v/v)=0:1-1:9) and then the resulting crude was passed through Pre-HPLC (instrument and preparative column: using SHIMADZU LC-20AP preparative liquid phase chromatographic instrument, preparative column model: Phenomenex C18). Preparation method: the crude was dissolved with methanol and dimethyl sulfoxide, and filtered with a 0.45 m filter membrane, to prepare into a sample liquid. Mobile phase system: acetonitrile/water (containing 10 mmol/L NH₄HCO₃). Gradient elution method: gradient elution with acetonitrile from 35% to 65% (elution time: 15 min). Lyophilization was performed to afford compound 23 (65 mg, yield: 27%).

¹H NMR (400 MHz, CDCl₃) δ 8.67 (s, 1H), 8.52-8.41 (m, 1H), 8.04-7.86 (m, 1H), 7.84-7.71 (m, 2H), 7.56-7.15 (m, 10H), 7.13-6.94 (m, 4H), 6.87-6.60 (m, 3H), 6.50-6.23 (m, 1H), 5.15-4.92 (m, 1H), 4.85-4.62 (m, 2H), 4.59-4.44 (m, 1H), 4.20-3.82 (m, 2H), 3.70-3.40 (m, 3H), 3.35-2.93 (m, 8H), 2.93-1.96 (m, 28H), 1.75-1.42 (m, 9H), 1.40-1.16 (m, 5H), 1.13-0.92 (m, 9H), 0.37 (s, 4H).

LCMS m/z=799.5 [M/2+1]⁺.

Example 24

(2S,4R)-1-((S)-2-(8-(4-((R)-3-((4-(N-(4-(4-((4′-chloro-2′-fluoro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (Compound 24)

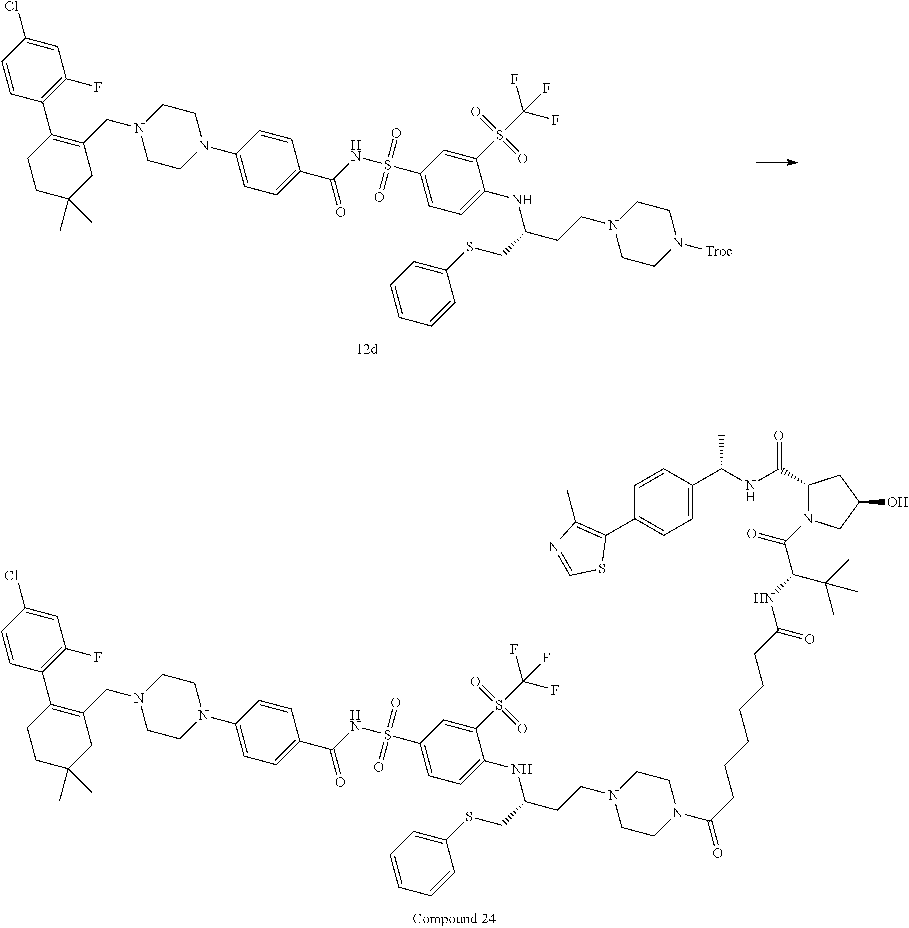

12d (5 g, 4.3 mmol) was dissolved in 200 mL of tetrahydrofuran. Acetic acid (16 g, 266.4 mmol) and zinc powder (16 g, 246.7 mmol) were sequentially added and the mixture was reacted at room temperature for 16 h. The reaction system was filtered and to the filtrate was added water (100 mL). The mixture was then extracted with 200 mL of ethyl acetate. The organic phase was separated, dried over anhydrous sodium sulfate and concentrated under reduced pressure to afford a crude (2 g). The above-mentioned crude (370 mg) was added to 8 mL of DCM. Intermediate 3 (220 mg, 0.35 mmol), triethylamine (0.51 mL, 3.6 mmol) and HATU (209 mg, 0.55 mmol) were sequentially added and the mixture was reacted at room temperature for 2 h. To the reaction system was added 20 mL of water and the mixture was extracted with 20 mL of DCM twice. The organic phase was dried over anhydrous sodium sulfate and concentrated under reduced pressure. Then the resulting crude was passed through Pre-HPLC (instrument and preparative column: using SHIMADZU LC-20AP preparative liquid phase chromatographic instrument, preparative column model: Phenomenex C18). Preparation method: the crude was dissolved with methanol and dimethyl sulfoxide, and filtered with a 0.45 m filter membrane, to prepare into a sample liquid. Mobile phase system: acetonitrile/water (containing 0.05% NH₄HCO₃). Gradient elution method: gradient elution with acetonitrile from 20% to 50% (elution time: 15 min). Lyophilization was performed to afford compound 24 (221 mg, yield: 18%).

¹H NMR (400 MHz, CDCl₃) δ 8.67 (s, 1H), 8.38-8.32 (m, 1H), 8.09 (dd, 1H), 7.68 (d, 2H), 7.48-7.22 (m, 10H), 7.16-6.90 (m, 4H), 6.82-6.71 (m, 2H), 6.62 (d, 1H), 6.26 (d, 1H), 5.15-5.00 (m, 1H), 4.80-4.67 (m, 1H), 4.60 (d, 1H), 4.55-4.45 (m, 1H), 4.15-4.07 (m, 1H), 3.97-3.82 (m, 1H), 3.73-3.54 (m, 2H), 3.48-3.20 (m, 7H), 3.18-2.92 (m, 2H), 2.75 (s, 2H), 2.57-1.90 (m, 24H), 1.76-1.40 (m, 10H), 1.36-1.23 (m, 4H), 1.09-0.93 (m, 15H).

LCMS m/z=788.0 [M/2+1]⁺.

Example 25

(2S,4R)-1-((S)-2-(8-((1S,4S)-5-((R)-3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)-2,5-diazabicyclo[2.2.1]heptan-2-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (Compound 25)

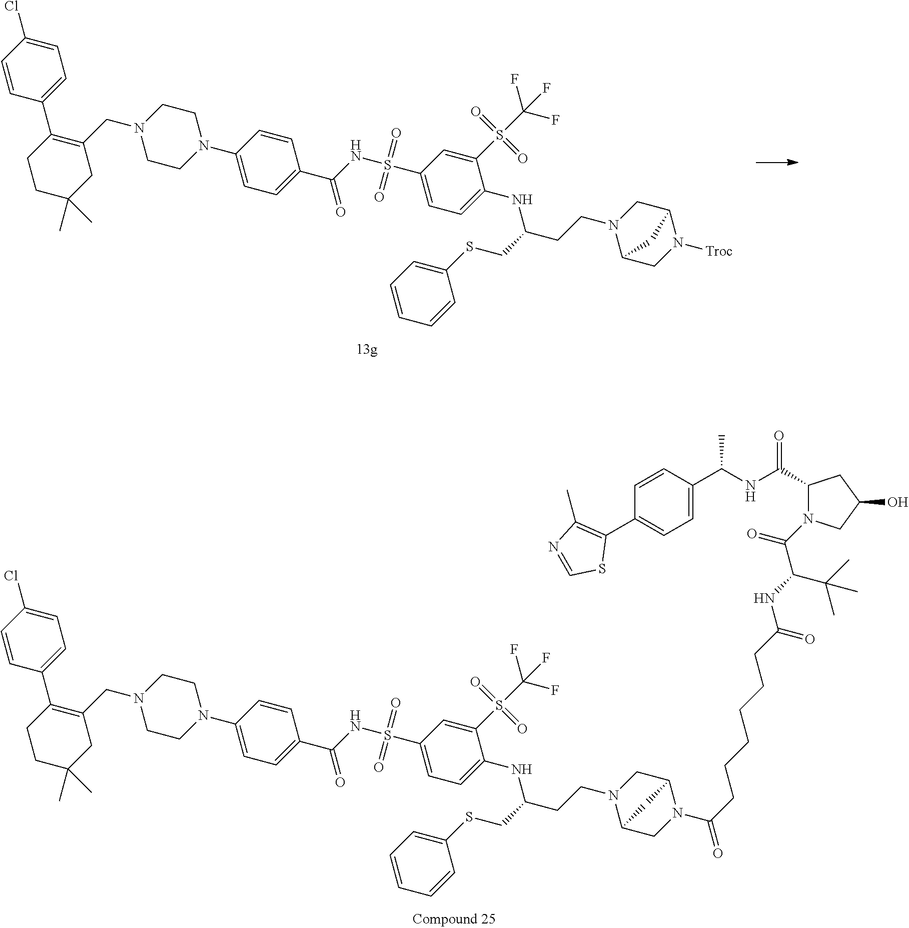

13g (0.5 g, 0.43 mmol) was added to 60 mL of tetrahydrofuran. Zinc powder (1.6 g, 24.46 mmol) and 1.6 mL of acetic acid were sequentially added and the mixture was reacted at room temperature for 4 h. To the reaction solution was added 25 mL of water to quench the reaction. The mixture was filtered over diatomaceous earth and the filtrate was adjusted to pH 8 with saturated aqueous sodium bicarbonate solution and extracted with 6 mL of dichloromethane twice. The organic phases were combined, dried over anhydrous sodium sulfate and concentrated under reduced pressure and the resulting crude was separated and purified by chromatographic column on silica gel (dichloromethane:methanol:triethylamine (v/v)=100:10:1) to afford a crude (0.2 g). The above-mentioned crude (0.20 g) was added to 5 mL of DCM. Intermediate 3 (0.12 g, 0.2 mmol), triethylamine (0.28 mL, 2.0 mmol) and HATU (0.11 g, 0.29 mmol) were sequentially added and the mixture was reacted at room temperature for 3 h. To the reaction system was added 15 mL of water and the mixture was extracted with 20 mL of DCM, dried over anhydrous sodium sulfate and concentrated under reduced pressure. Then the resulting crude was passed through Pre-HPLC (instrument and preparative column: using SHIMADZU LC-20AP preparative liquid phase chromatographic instrument, preparative column model: Phenomenex C18). Preparation method: the crude was dissolved with methanol and dimethyl sulfoxide, and filtered with a 0.45 m filter membrane, to prepare into a sample liquid. Mobile phase system: acetonitrile/water (containing 0.05% NH₄HCO₃). Gradient elution method: gradient elution with acetonitrile from 50% to 80% (elution time: 15 min). Lyophilization was performed to afford compound 25 (97 mg, yield: 14%).

¹H NMR (400 MHz, CDCl₃) δ 8.66 (s, 1H), 8.44-8.32 (m, 1H), 8.07-7.95 (m, 1H), 7.80-7.67 (m, 2H), 7.54-7.16 (m, 13H), 7.03-6.94 (m, 2H), 6.83-6.70 (m, 2H), 6.69-6.55 (m, 1H), 6.39-6.25 (m, 1H), 5.15-4.96 (m, 1H), 4.80-4.18 (m, 4H), 4.16-4.05 (m, 1H), 4.03-3.87 (m, 1H), 3.63-3.53 (m, 1H), 3.53-3.35 (m, 2H), 3.33-2.72 (m, 10H), 2.70-1.95 (m, 21H), 1.95-1.20 (m, 16H), 1.15-0.86 (m, 15H).

LCMS m/z=785.0 [M/2+1]⁺.

Example 26

cis-(2S,4R)-1-((S)-2-(7-(5-((R)-3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)-3a,6a-dimethylhexahydropyrrolo[3,4-c]pyrrol-2(1H)-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 26)

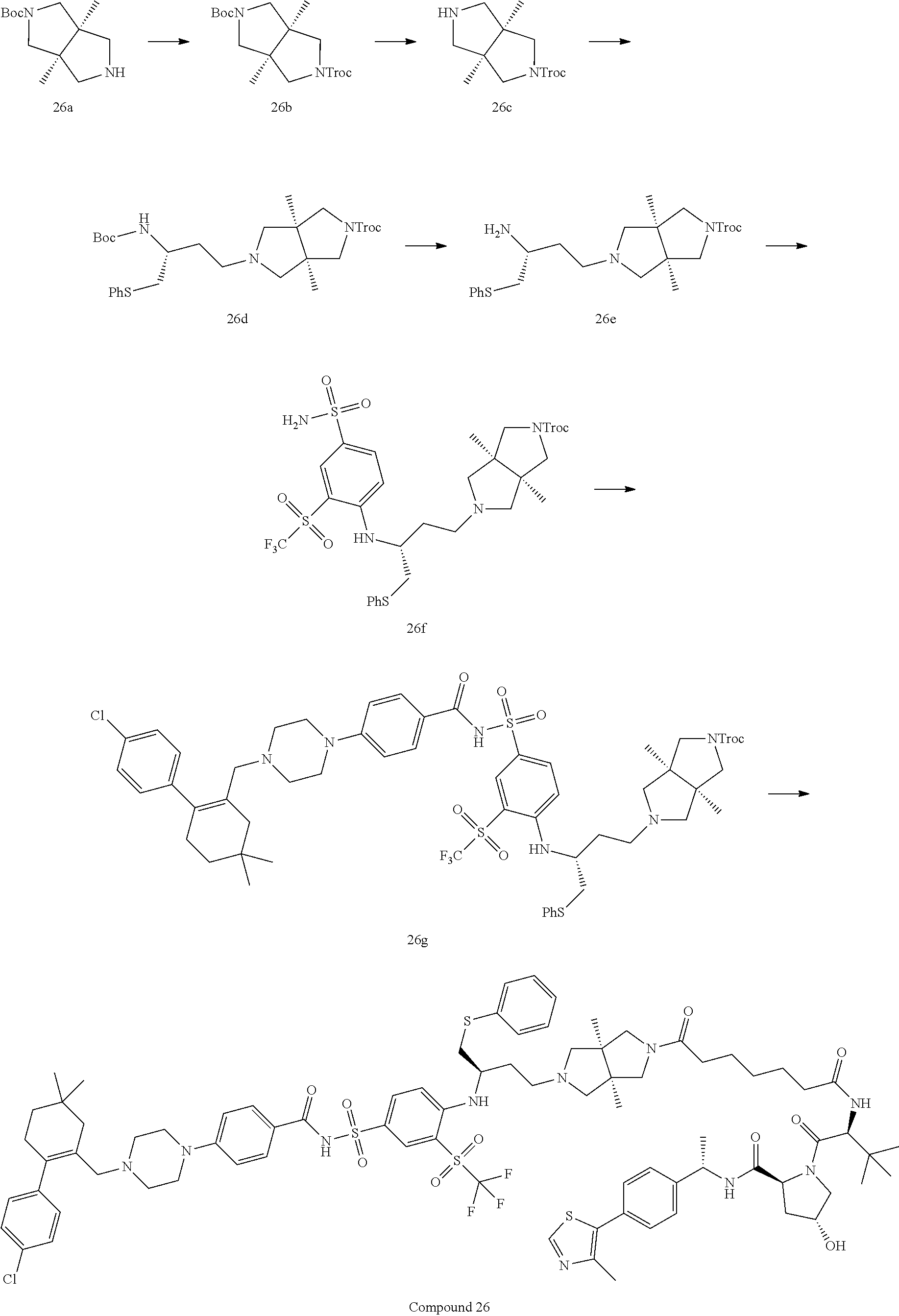

### Step 1: Preparation of 26b

26a (1.0 g, 4.16 mmol) was dissolved in 10 mL of DCM and triethylamine (0.88 g, 8.70 mmol) was added. The mixture was cooled to 0° C. and 2,2,2-trichloroethyl chloroformate (0.97 g, 4.58 mmol) was slowly added dropwise. Then the mixture was slowly warmed to room temperature and reacted for 2 h. After the reaction was completed, the reaction solution was diluted with 40 mL of DCM and then 50 mL of water was added. The organic phase was separated, dried over anhydrous sodium sulfate and concentrated under reduced pressure and the resulting crude was separated and purified by silica gel column chromatography (petroleum ether/ethyl acetate (v/v)=10:1-2:1) to afford 26b (1.7 g, yield: 98%).

### Step 2: Preparation of 26c

26b (1.65 g, 3.97 mmol) was dissolved in 10 mL of DCM. Trifluoroacetic acid (2.9 mL) was added and the mixture was reacted at room temperature for 3 h. The reaction solution was concentrated under reduced pressure. To the residue were added 20 mL of DCM and 20 mL of water. The mixture was adjusted to pH 9 with saturated sodium bicarbonate solution. The organic phase was separated, dried over anhydrous sodium sulfate and concentrated under reduced pressure to afford crude 26c (1.5 g).

### Step 3: Preparation of 26d

The above-mentioned crude 26c (1.5 g) was added to 10 mL of 1,2-dichloroethane. Tert-butyl (R)-(4-oxo-1-(phenylthio)butan-2-yl)carbamate (0.94 g, 3.18 mmol) and glacial acetic acid (0.18 mL) were added and the mixture was reacted at room temperature for 0.5 h. Sodium triacetoxyborohydride (2.2 g, 10.38 mmol) was added and the resulting mixture was reacted at room temperature for 19 h. DCM (50 mL) was added to dilute the reaction solution. Saturated sodium bicarbonate solution was used to adjust the reaction mixture to pH 9. The organic phase was separated, dried over anhydrous sodium sulfate and concentrated under reduced pressure. The resulting crude was separated and purified by chromatographic column on silica gel (dichloromethane:methanol (v/v)=10:1) to afford 26d (0.68 g, yield over two steps from compound 26b: 29%).

### Step 4: Preparation of Trifluoroacetate of 26e

26d (0.68 g, 1.14 mmol) was dissolved in 4 mL of DCM. Trifluoroacetic acid (2 mL) was added and the mixture was reacted at room temperature for 3 h. The reaction solution was concentrated under reduced pressure to afford crude trifluoroacetate of 26e (0.6 g).

### Step 5: Preparation of 26f

The above-mentioned crude trifluoroacetate of 26e (0.6 g) was added to 4 mL of acetonitrile. Triethylamine (0.79 mL, 5.67 mmol) and 4-fluoro-3-((trifluoromethyl)sulfonyl)benzenesulfonamide (0.35 g, 1.14 mmol) were added and the mixture was refluxed and reacted at 80° C. for 3 h. The reaction mixture was cooled to room temperature. The reaction solution was concentrated under reduced pressure and the resulting crude was separated and purified by chromatographic column on silica gel (dichloromethane:methanol (v/v)=10:1) to afford 26f (0.87 g, yield over two steps from compound 26d: 98%).

### Step 6: 26g

26f (0.87 g, 1.11 mmol) was added to 10 mL of DCM. 4-(4-((4′-chloro-4,4-dimethyl-3,4,5,6-tetrahydro-[1,1′-biphenyl]-2-yl)methyl)piperazin-1-yl)benzoic acid (0.49 g, 1.11 mmol), DMAP (0.27 g, 2.21 mmol) and EDCI (0.43 g, 2.25 mmol) were sequentially added and the mixture was reacted at room temperature for 12 h. To the reaction system was added 20 mL of water. The organic phase was separated, dried over anhydrous sodium sulfate and concentrated under reduced pressure and the resulting crude was separated and purified by chromatographic column on silica gel (dichloromethane:methanol (v/v)=10:1) to afford 26g (0.80 g, yield: 60%).

### Step 7: Preparation of Compound 26

26g (0.25 g, 0.208 mmol) was added to 5 mL of mixed solvents of methanol/tetrahydrofuran ((v/v)=10:1). Zinc powder (1.1 g, 16.9 mmol) and ammonium chloride (0.34 g, 6.36 mmol) were sequentially added and the mixture was stirred at 30° C. for 19 h. The reaction system was filtered and the filtrate was concentrated under reduced pressure. The crude was separated and purified by chromatographic column on silica gel (dichloromethane:methanol (v/v)=10:1) to afford a crude (0.08 g). The above-mentioned crude (0.08 g) was added to 8 mL of DCM. Intermediate 1 (50 mg, 0.085 mmol), triethylamine (0.12 mL, 0.86 mmol) and HATU (48 mg, 0.126 mmol) were sequentially added and the mixture was reacted at room temperature for 2 h. To the reaction system was added 20 mL of water and the mixture was extracted with 30 mL of DCM twice. The organic phase was separated, dried over anhydrous sodium sulfate and concentrated under reduced pressure. Then the resulting crude was passed through Pre-HPLC (instrument and preparative column: using SHIMADZU LC-20AP preparative liquid phase chromatographic instrument, preparative column model: Phenomenex). Preparation method: the crude was dissolved with methanol and dimethyl sulfoxide, and filtered with a 0.45 m filter membrane, to prepare into a sample liquid. Mobile phase system: acetonitrile/water. Gradient elution method: gradient elution with acetonitrile from 59% to 89% (elution time: 15 min). Lyophilization was performed to afford compound 26 (80 mg, yield: 24%).

¹H NMR (400 MHz, CDCl₃) δ 8.73-8.62 (m, 1H), 8.55-8.45 (m, 1H), 8.00-7.70 (m, 3H), 7.44-7.15 (m, 12H), 7.05-6.70 (m, 6H), 6.63-6.34 (m, 1H), 5.17-4.60 (m, 3H), 4.58-4.45 (m, 1H), 4.25-3.90 (m, 2H), 3.75-2.62 (m, 15H), 2.60-1.91 (m, 20H), 1.65-0.75 (m, 35H).

LCMS m/z=799.0 [M/2+1]⁺.

Example 27

(2S,4R)-1-((S)-2-(7-((1S,4S)-5-((R)-3-((4-(N-(4-(4-((6-(4-chlorophenyl)spiro[2.5]oct-5-en-5-yl)methyl)piperazin-1-yl)benzoyl)sulfamoyl)-2-((trifluoromethyl)sulfonyl)phenyl)amino)-4-(phenylthio)butyl)-2,5-diazabicyclo[2.2.1]heptan-2-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 27)

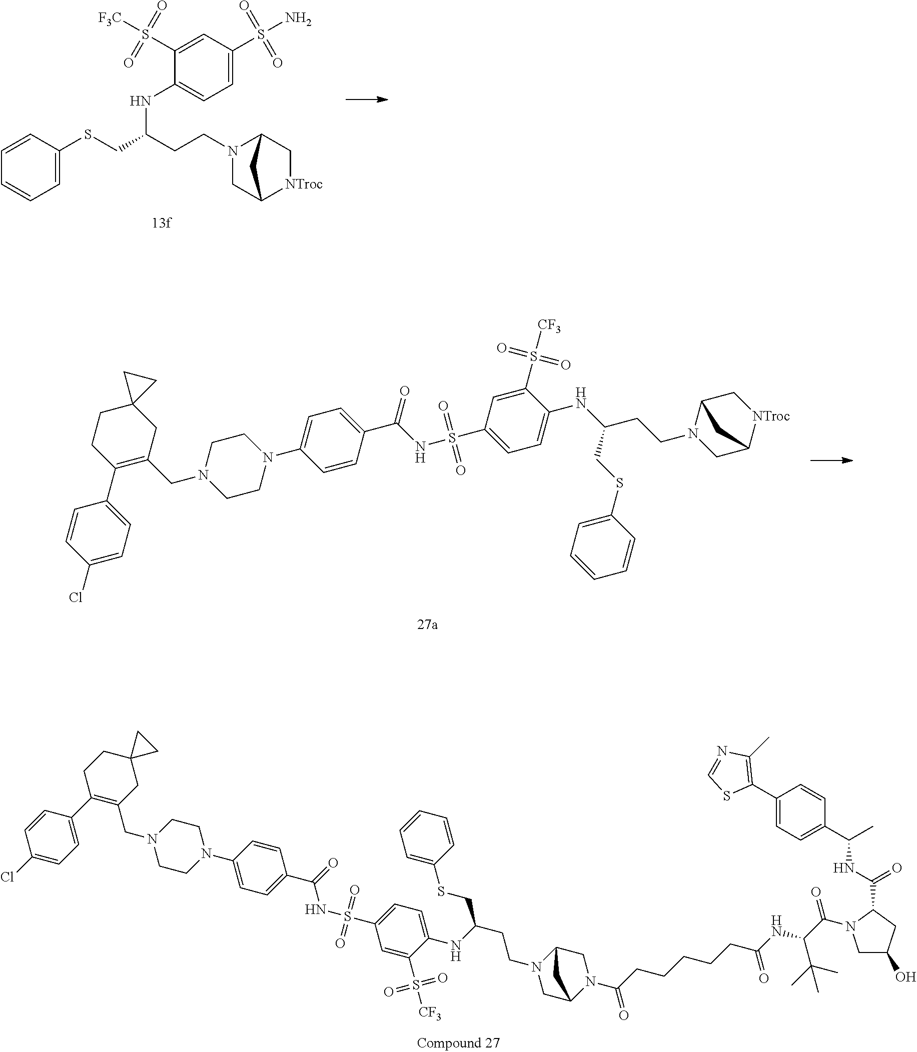

### Step 1: Preparation of 27a

13f (0.50 g, 0.68 mmol) was added to 6 mL of DCM. 11a (0.30 g, 0.687 mmol), DMAP (0.17 g, 1.39 mmol) and EDCI (0.26 g, 1.36 mmol) were sequentially added and the mixture was reacted at 35° C. for 12 h. The reaction system was cooled to room temperature and 5 mL of water and 3 mL of dichloromethane were added, followed by liquid separation. The aqueous phase was extracted with dichloromethane (5 mL×3). The organic phases were combined, dried over anhydrous sodium sulfate and concentrated under reduced pressure and the resulting crude was separated and purified by chromatographic column on silica gel (dichloromethane:methanol (v/v)=10:1) to afford 27a (0.30 g, yield: 38%).

LCMS m/z=1159.1 [M+1]⁺.

### Step 2: Preparation of Compound 27

27a (0.30 g, 0.26 mmol) was added to 3 mL of tetrahydrofuran and 15 mL of methanol. Zinc powder (1.36 g, 20.9 mmol) and ammonium chloride (0.42 g, 7.85 mmol) were sequentially added and the mixture was stirred at 27° C. for 12 h. To the reaction solution was added 50 mL of ethyl acetate and 30 mL of water. The mixture was filtered over diatomaceous earth and the filtrate was extracted with ethyl acetate (15 mL×2). The organic phases were combined, dried over anhydrous sodium sulfate and concentrated under reduced pressure to afford a crude (0.23 g). The above-mentioned crude (0.13 g) was added to 5 mL of DCM. Intermediate 1 (0.076 g, 0.13 mmol), triethylamine (0.066 g, 0.65 mmol) and HATU (0.074 g, 0.195 mmol) were sequentially added and the mixture was reacted at room temperature for 2 h. To the reaction solution was added 5 mL of water, followed by liquid separation. The aqueous phase was extracted with DCM (6 mL×2) and the organic phases were combined, dried over anhydrous sodium sulfate and concentrated under reduced pressure. The resulting crude was separated and purified by chromatographic column on silica gel (dichloromethane:methanol (v/v)=10:1) to afford a crude, which was then passed through Pre-HPLC (instrument and preparative column: using SHIMADZU LC-20AP preparative liquid phase chromatographic instrument, preparative column model: Phenomenex C18). Mobile phase system: acetonitrile/water (containing 0.05% NH₄HCO₃). Gradient elution method: gradient elution with acetonitrile from 50% to 80% (elution time: 15 min). Lyophilization was performed to afford compound 27 (0.10 g, yield: 44%).

¹H NMR (400 MHz, CDCl₃) δ 8.60 (s, 1H), 8.39-8.28 (m, 1H), 8.02-7.84 (m, 1H), 7.74-7.56 (m, 2H), 7.40-6.90 (m, 15H), 6.79-6.51 (m, 3H), 6.42-6.20 (m, 1H), 5.06-4.85 (m, 1H), 4.75-4.10 (m, 4H), 4.10-3.80 (m, 2H), 3.61-2.70 (m, 12H), 2.70-1.86 (m, 22H), 1.75-1.15 (m, 14H), 1.07-0.91 (m, 9H), 0.29 (s, 4H).

LCMS m/z=776.5 [M/2+1]⁺.

Example 28

(2S,4R)-1-((S)-2-(8-((1S,4S)-5-((R)-3-((4-(N-(4-(4-((6-(4-chlorophenyl)spiro[2.5]oct-5-en-5-yl)methyl)piperazin-1-yl)benzoyl)sulfamoyl)-2-((trifluoromethyl)sulfonyl)phenyl)amino)-4-(phenylthio)butyl)-2,5-diazabicyclo[2.2.1]heptan-2-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (Compound 28)

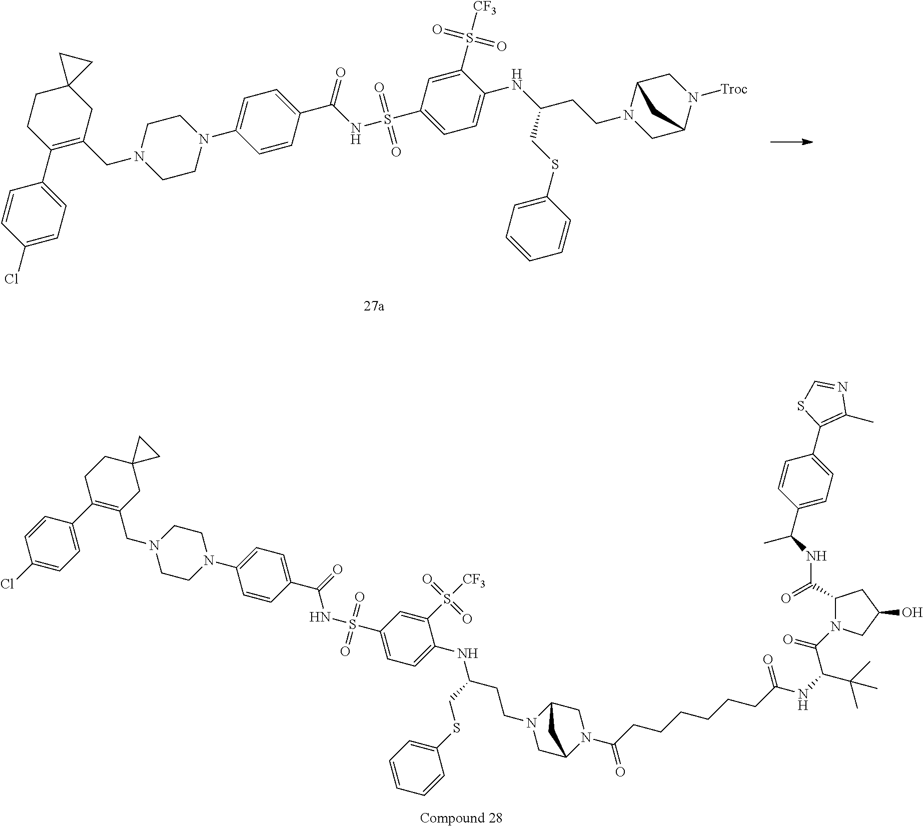

27a (0.30 g, 0.26 mmol) was added to 3 mL of tetrahydrofuran and 15 mL of methanol. Zinc powder (1.36 g, 20.9 mmol) and ammonium chloride (0.42 g, 7.85 mmol) were sequentially added and the mixture was stirred at 27° C. for 12 h. To the reaction solution was added 50 mL of ethyl acetate and 30 mL of water. The mixture was filtered over diatomaceous earth and the filtrate was extracted with ethyl acetate (15 mL×2). The organic phases were combined, dried over anhydrous sodium sulfate and concentrated under reduced pressure to afford a crude (0.23 g). The above-mentioned crude (0.10 g) was added to 5 mL of DCM. Intermediate 3 (0.060 g, 0.10 mmol), triethylamine (0.051 g, 0.50 mmol) and HATU (0.057 g, 0.15 mmol) were sequentially added and the mixture was reacted at room temperature for 2 h. To the reaction solution was added 5 mL of water, followed by liquid separation. The aqueous phase was extracted with DCM (6 mL×2) and the organic phases were combined, dried over anhydrous sodium sulfate and concentrated under reduced pressure. The resulting crude was separated and purified by chromatographic column on silica gel (dichloromethane:methanol (v/v)=10:1) to afford a crude, which was then passed through Pre-HPLC (instrument and preparative column: using SHIMADZU LC-20AP preparative liquid phase chromatographic instrument, preparative column model: Phenomenex C18). Mobile phase system: acetonitrile/water (containing 0.05% NH₄HCO₃). Gradient elution method: gradient elution with acetonitrile from 50% to 80% (elution time: 15 min). Lyophilization was performed to afford compound 28 (60 mg, yield: 34%).

¹H NMR (400 MHz, CDCl₃) δ 8.67 (s, 1H), 8.38 (s, 1H), 8.10-7.92 (m, 1H), 7.80-7.66 (m, 2H), 7.49-7.12 (m, 13H), 7.07-6.98 (m, 2H), 6.83-6.58 (m, 3H), 6.38-6.24 (m, 1H), 5.15-4.97 (m, 1H), 4.83-4.20 (m, 4H), 4.16-3.90 (m, 2H), 3.68-2.75 (m, 13H), 2.75-1.97 (m, 21H), 1.97-1.17 (m, 16H), 1.14-0.90 (m, 9H), 0.36 (s, 4H).

LCMS m/z=783.5 [M/2+1]⁺.

Example 29

(2S,4R)-1-((S)-2-(7-((1S,4S)-5-((R)-3-((4-(N-(4-(4-((6-(4-chloro-2-fluorophenyl)spiro[2.5]oct-5-en-5-yl)methyl)piperazin-1-yl)benzoyl)sulfamoyl)-2-((trifluoromethyl)sulfonyl)phenyl)amino)-4-(phenylthio)butyl)-2,5-diazabicyclo[2.2.1]heptan-2-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 29)

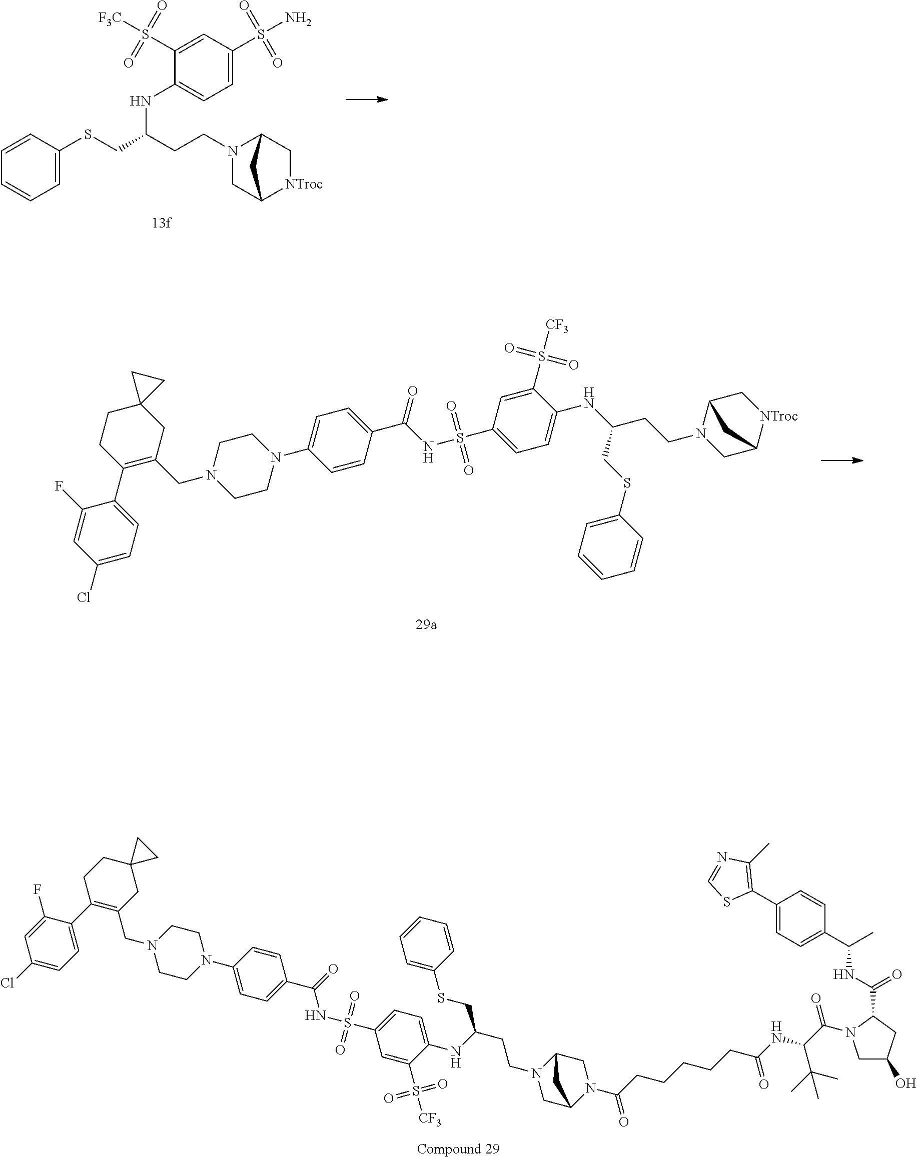

### Step 1: Preparation of 29a

Sodium hydroxide (0.26 g, 6.5 mmol) was added to 5 mL of methanol and 3 mL of water. A solution of 21c (1.27 g, 2.63 mmol) in 10 mL of tetrahydrofuran was added and the resulting mixture was reacted at 80° C. for 6 h. The reaction solution was cooled to room temperature and concentrated under reduced pressure. The residue was adjusted to pH 5 with 1 mol/L hydrochloric acid and extracted with ethyl acetate (15 mL×3). The organic phase was washed with 20 mL of saturated sodium chloride solution, dried over anhydrous sodium sulfate and concentrated under reduced pressure to afford a crude (0.96 g). 13f (0.50 g, 0.68 mmol) was added to 6 mL of DCM. The above-mentioned crude (0.31 g), DMAP (0.17 g, 1.36 mmol) and EDCI (0.26, 1.36 mmol) were sequentially added and the mixture was warmed to 35° C. and reacted for 12 h. The reaction system was cooled to room temperature and 5 mL of water and 3 mL of DCM were added, followed by liquid separation. The aqueous phase was extracted with DCM (5 mL×3). The organic phases were combined, dried over anhydrous sodium sulfate and concentrated under reduced pressure and the resulting crude was separated and purified by chromatographic column on silica gel (dichloromethane methanol (v/v)=10:1) to afford 29a (0.35 g, yield: 44%).

LCMS m/z=1177.3 [M+1]⁺.

### Step 2: Preparation of Compound 29

29a (0.35 g, 0.30 mmol) was added to 3 mL of tetrahydrofuran and 15 mL of methanol. Zinc powder (1.57 g, 24.15 mmol) and ammonium chloride (0.48 g, 8.97 mmol) were sequentially added and the mixture was stirred at 27° C. for 12 h. To the reaction solution was added 50 mL of ethyl acetate and 30 mL of water. The mixture was filtered over diatomaceous earth and the filtrate was extracted with ethyl acetate (15 mL×2). The organic phases were combined, dried over anhydrous sodium sulfate and concentrated under reduced pressure to afford a crude (0.24 g). The above-mentioned crude (0.14 g) was added to 5 mL of DCM. Intermediate 1 (0.082 g, 0.14 mmol), triethylamine (0.071 g, 0.70 mmol) and HATU (0.080 g, 0.21 mmol) were sequentially added and the mixture was reacted at room temperature for 2 h. To the reaction solution was added 5 mL of water, followed by liquid separation. The aqueous phase was extracted with DCM (6 mL×2) and the organic phases were combined, dried over anhydrous sodium sulfate and concentrated under reduced pressure. The resulting crude was separated and purified by chromatographic column on silica gel (dichloromethane:methanol (v/v)=10:1) to afford a crude, which was then passed through Pre-HPLC (instrument and preparative column: using SHIMADZU LC-20AP preparative liquid phase chromatographic instrument, preparative column model: Phenomenex C18). Mobile phase system: acetonitrile/water (containing 0.05% NH₄HCO₃). Gradient elution method: gradient elution with acetonitrile from 50% to 80% (elution time: 15 min). Lyophilization was performed to afford compound 29 (85 mg, yield: 31%).

¹H NMR (400 MHz, CDCl₃) δ 8.67 (s, 1H), 8.45-8.35 (m, 1H), 8.08-7.92 (m, 1H), 7.84-7.64 (m, 2H), 7.44-6.94 (m, 14H), 6.82-6.58 (m, 3H), 6.50-6.30 (m, 1H), 5.17-4.90 (m, 1H), 4.85-4.15 (m, 4H), 4.15-3.87 (m, 2H), 3.80-2.92 (m, 10H), 2.90-1.80 (m, 24H), 1.80-1.20 (m, 14H), 1.04 (s, 9H), 0.37 (s, 4H).

LCMS m/z=785.5 [M/2+1]⁺.

Example 30

(2S,4R)-1-((S)-2-(8-((1S,4S)-5-((R)-3-((4-(N-(4-(4-((6-(4-chloro-2-fluorophenyl)spiro[2.5]oct-5-en-5-yl)methyl)piperazin-1-yl)benzoyl)sulfamoyl)-2-((trifluoromethyl)sulfonyl)phenyl)amino)-4-(phenylthio)butyl)-2,5-diazabicyclo[2.2.1]heptan-2-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (Compound 30)

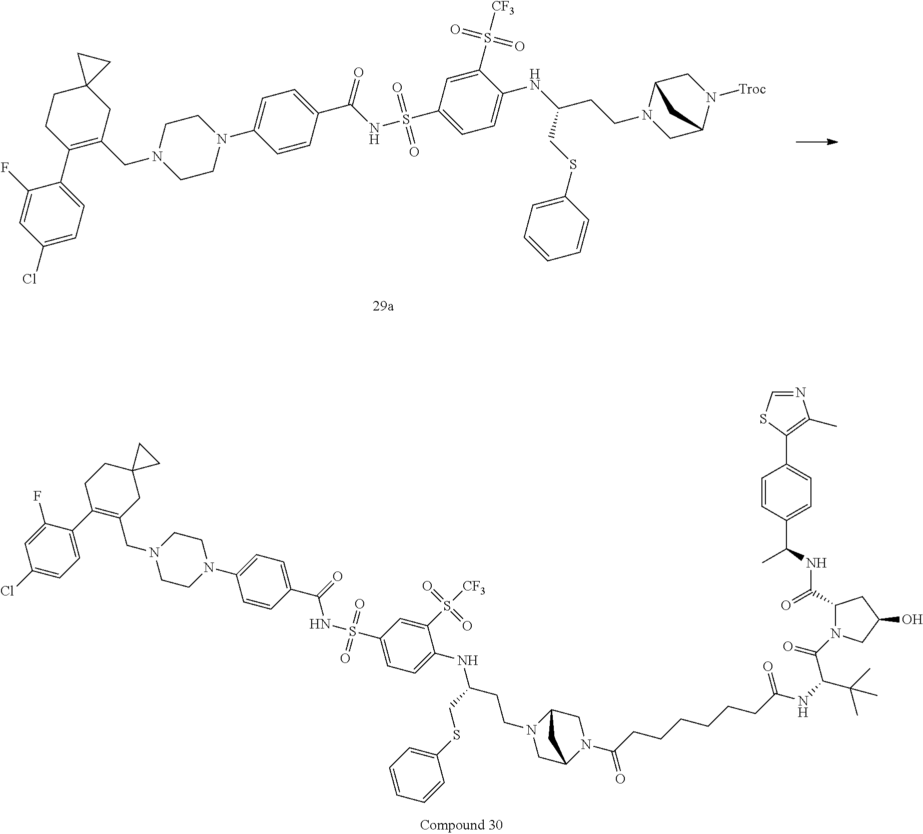

29a (0.35 g, 0.30 mmol) was added to 3 mL of tetrahydrofuran and 15 mL of methanol. Zinc powder (1.57 g, 24.15 mmol) and ammonium chloride (0.48 g, 8.97 mmol) were sequentially added and the mixture was stirred at 27° C. for 12 h. To the reaction solution was added 50 mL of ethyl acetate and 30 mL of water. The mixture was filtered over diatomaceous earth and the filtrate was extracted with ethyl acetate (15 mL×2). The organic phases were combined, dried over anhydrous sodium sulfate and concentrated under reduced pressure to afford a crude (0.24 g). The above-mentioned crude (0.10 g) was added to 5 mL of DCM. Intermediate 3 (0.060 g, 0.10 mmol), triethylamine (0.051 g, 0.50 mmol) and HATU (0.057 g, 0.15 mmol) were sequentially added and the mixture was reacted at room temperature for 2 h. To the reaction solution was added 5 mL of water, followed by liquid separation. The aqueous phase was extracted with DCM (6 mL×2) and the organic phases were combined, dried over anhydrous sodium sulfate and concentrated under reduced pressure. The resulting crude was separated and purified by chromatographic column on silica gel (dichloromethane:methanol (v/v)=10:1) to afford a crude, which was then passed through Pre-HPLC (instrument and preparative column: using SHIMADZU LC-20AP preparative liquid phase chromatographic instrument, preparative column model: Phenomenex C18). Mobile phase system: acetonitrile/water (containing 10 mmol/L NH₄HCO₃). Gradient elution method: gradient elution with acetonitrile from 35% to 65% (elution time: 15 min). Lyophilization was performed to afford compound 30 (75 mg, yield: 38%).

¹H NMR (400 MHz, CDCl₃) δ 8.66 (s, 1H), 8.41-8.36 (m, 1H), 8.09-7.94 (m, 1H), 7.78-7.66 (m, 2H), 7.48-6.95 (m, 14H), 6.82-6.58 (m, 3H), 6.35-6.24 (m, 1H), 5.15-4.97 (m, 1H), 4.81-4.20 (m, 4H), 4.16-3.90 (m, 2H), 3.65-2.95 (m, 10H), 2.90-1.97 (m, 24H), 1.80-1.15 (m, 16H), 1.12-0.95 (m, 9H), 0.37 (s, 4H).

LCMS m/z=792.5 [M/2+1]⁺.

Example 31

(2S,4R)-1-((S)-2-(7-(4-((R)-3-((4-(N-(4-(4-((6-(4-chloro-2-fluorophenyl)spiro[2.5]oct-5-en-5-yl)methyl)piperazin-1-yl)benzoyl)sulfamoyl)-2-((trifluoromethyl)sulfonyl)phenyl)amino)-4-(phenylthio)butyl)piperazin-1-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 31)

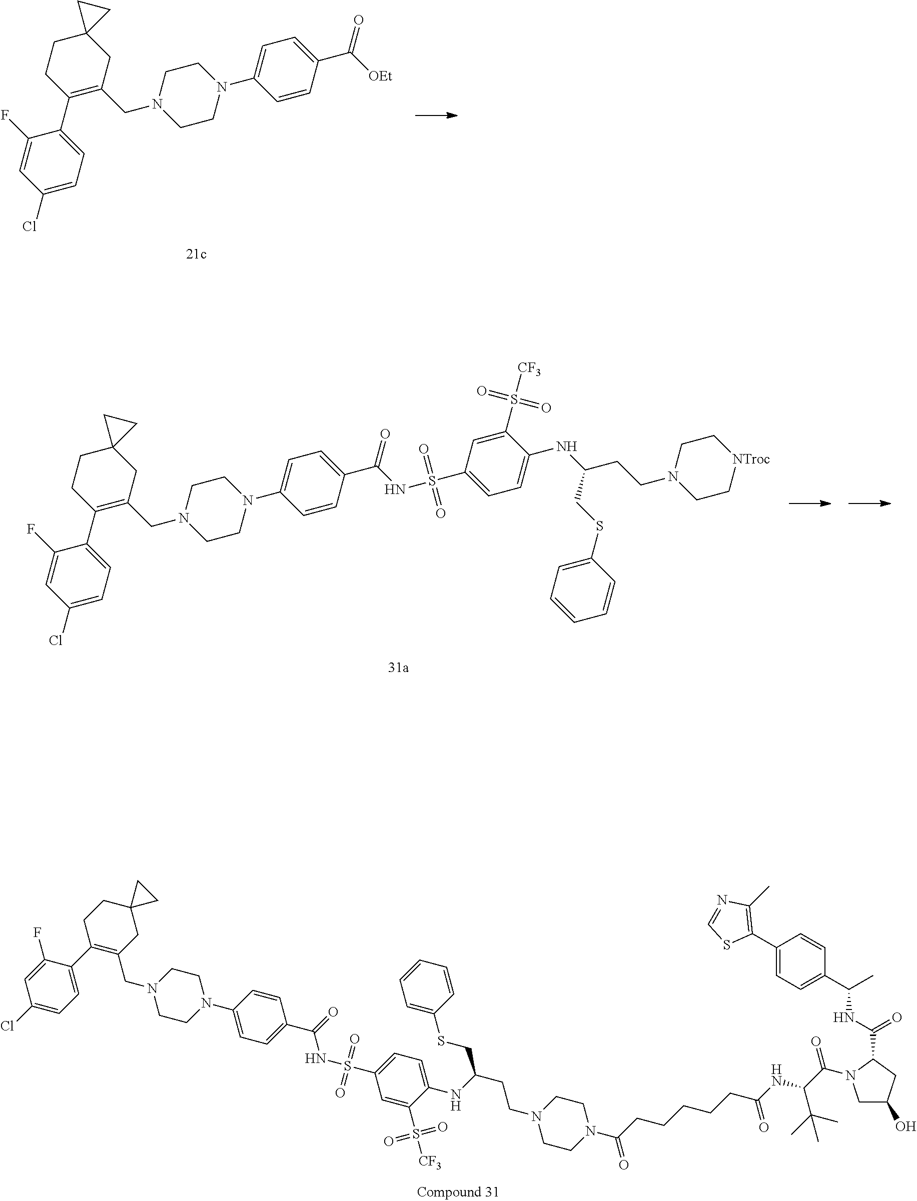

### Step 1: Preparation of 31a

Sodium hydroxide (0.26 g, 6.5 mmol) was added to 5 mL of methanol and 3 mL of water. A solution of 21c (1.27 g, 2.63 mmol) in 10 mL of tetrahydrofuran was added and the resulting mixture was reacted at 80° C. for 6 h. The reaction solution was cooled to room temperature and concentrated under reduced pressure. The residue was adjusted to pH 5 with 1 mol/L hydrochloric acid and extracted with ethyl acetate (15 mL×3). The organic phase was washed with 20 mL of saturated sodium chloride solution, dried over anhydrous sodium sulfate and concentrated under reduced pressure to afford a crude (0.96 g). Intermediate 2 (0.62 g, 0.85 mmol) was added to 6 mL of DCM. The above-mentioned crude (0.43 g), DMAP (0.21 g, 1.72 mmol) and EDCI (0.33 g, 1.73 mmol) were sequentially added and the mixture was reacted at 35° C. for 12 h. The reaction system was cooled to room temperature and 5 mL of water and 3 mL of DCM were added, followed by liquid separation. The aqueous phase was extracted with DCM (5 mL×3). The organic phases were combined, dried over anhydrous sodium sulfate and concentrated under reduced pressure and the resulting crude was separated and purified by chromatographic column on silica gel (dichloromethane methanol (v/v)=10:1) to afford 31a (0.70 g, yield: 71%).

LCMS m/z=583.2 [M/2+1]⁺.

### Step 2: Preparation of Compound 31

31a (0.70 g, 0.6 mmol) was added to 5 mL of tetrahydrofuran and 15 mL of methanol. Zinc powder (3.14 g, 48.31 mmol) and ammonium chloride (0.96 g, 17.95 mmol) were sequentially added and the mixture was stirred at 27° C. for 12 h. To the reaction solution was added 50 mL of ethyl acetate and 30 mL of water. The mixture was filtered over diatomaceous earth and the filtrate was extracted with ethyl acetate (15 mL×2). The organic phases were combined, dried over anhydrous sodium sulfate and concentrated under reduced pressure to afford a crude (0.41 g). The above-mentioned crude (0.14 g) was added to 5 mL of DCM. Intermediate 1 (0.082 g, 0.14 mmol), triethylamine (0.071 g, 0.70 mmol) and HATU (0.080 g, 0.21 mmol) were sequentially added and the mixture was reacted at room temperature for 2 h. To the reaction solution was added 5 mL of water, followed by extraction using DCM (6 mL×2). The organic phases were combined, dried over anhydrous sodium sulfate and concentrated under reduced pressure. The resulting crude was separated and purified by chromatographic column on silica gel (dichloromethane:methanol (v/v)=10:1) to afford a crude, which was then passed through Pre-HPLC (instrument and preparative column: using SHIMADZU LC-20AP preparative liquid phase chromatographic instrument, preparative column model: Phenomenex C18, mobile phase system: acetonitrile/water (containing 0.05% NH₄HCO₃), gradient elution method: gradient elution with acetonitrile from 50% to 80% (elution time: 15 min)). Lyophilization was performed to afford compound 31 (50 mg, yield: 16%).

¹H NMR (400 MHz, CDCl₃) δ 8.67 (s, 1H), 8.40-8.32 (m, 1H), 8.09 (dd, 1H), 7.73-7.65 (m, 2H), 7.46-7.20 (m, 10H), 7.16-6.96 (m, 4H), 6.81-6.71 (m, 2H), 6.68-6.59 (m, 1H), 6.36-6.25 (m, 1H), 5.15-5.00 (m, 1H), 4.80-4.68 (m, 1H), 4.65-4.44 (m, 2H), 4.15-4.03 (m, 1H), 4.00-3.83 (m, 1H), 3.75-2.70 (m, 13H), 2.60-1.95 (m, 24H), 1.77-1.20 (m, 12H), 1.04 (s, 9H), 0.37 (s, 4H).

LCMS m/z=779.5 [M/2+1]⁺.

Example 32

(2S,4R)-1-((S)-2-(8-(4-((R)-3-((4-(N-(4-(4-((6-(4-chloro-2-fluorophenyl)spiro[2.5]oct-5-en-5-yl)methyl)piperazin-1-yl)benzoyl)sulfamoyl)-2-((trifluoromethyl)sulfonyl)phenyl)amino)-4-(phenylthio)butyl)piperazin-1-yl)-8-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 32)

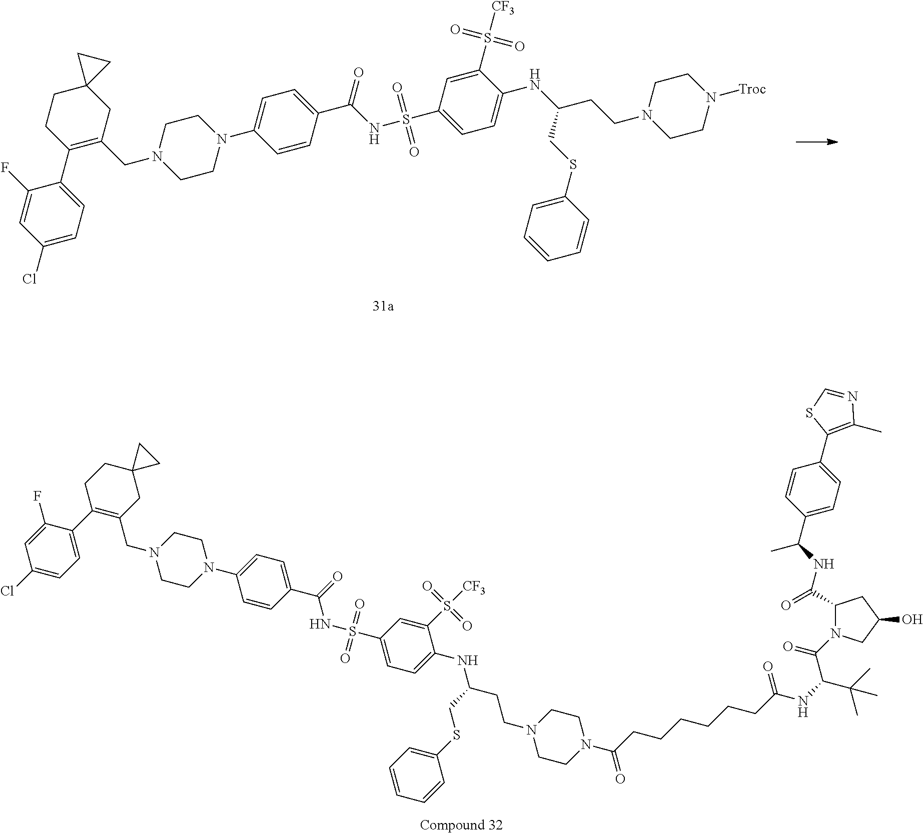

31a (0.70 g, 0.6 mmol) was added to 5 mL of tetrahydrofuran and 15 mL of methanol. Zinc powder (3.14 g, 48.31 mmol) and ammonium chloride (0.96 g, 17.95 mmol) were sequentially added and the mixture was stirred at 27° C. for 12 h. To the reaction solution was added 50 mL of ethyl acetate and 30 mL of water. The mixture was filtered over diatomaceous earth and the filtrate was extracted with ethyl acetate (15 mL×2). The organic phases were combined, dried over anhydrous sodium sulfate and concentrated under reduced pressure to afford a crude (0.41 g). The above-mentioned crude (0.10 g) was added to 5 mL of DCM. Intermediate 3 (0.060 g, 0.10 mmol), triethylamine (0.051 g, 0.504 mmol) and HATU (0.057 g, 0.15 mmol) were sequentially added and the mixture was reacted at room temperature for 2 h. To the reaction solution was added 5 mL of water, followed by extraction using DCM (6 mL×2). The organic phases were combined, dried over anhydrous sodium sulfate and concentrated under reduced pressure. The resulting crude was separated and purified by chromatographic column on silica gel (dichloromethane:methanol (v/v)=10:1) to afford a crude, which was then passed through Pre-HPLC (instrument and preparative column: using SHIMADZU LC-20AP preparative liquid phase chromatographic instrument, preparative column model: Phenomenex C18, mobile phase system: acetonitrile/water (containing 0.05% NH₄HCO₃), gradient elution method: gradient elution with acetonitrile from 50% to 80% (elution time: 15 min)). Lyophilization was performed to afford compound 32 (50 mg, yield: 22%).

¹H NMR (400 MHz, CDCl₃) δ 8.68 (s, 1H), 8.37-8.32 (m, 1H), 8.09 (dd, 1H), 7.74-7.65 (m, 2H), 7.46-7.20 (m, 10H), 7.14-6.96 (m, 4H), 6.82-6.70 (m, 2H), 6.66-6.56 (m, 1H), 6.36-6.24 (m, 1H), 5.15-5.00 (m, 1H), 4.80-4.43 (m, 3H), 4.16-4.04 (m, 1H), 3.98-3.82 (m, 1H), 3.73-3.54 (m, 2H), 3.50-3.20 (m, 7H), 3.17-2.92 (m, 2H), 2.78 (s, 2H), 2.60-1.95 (m, 24H), 1.78-1.40 (m, 10H), 1.36-1.20 (m, 4H), 1.04 (s, 9H), 0.36 (s, 4H).

LCMS m/z=786.5 [M/2+1]⁺.

Example 33

(2S,4R)-1-((S)-2-(7-((1S,4S)-5-((R)-3-((4-(N-(4-(4-((2-(4-chlorophenyl)cyclohept-1-en-1-yl)methyl)piperazin-1-yl)benzoyl)sulfamoyl)-2-((trifluoromethyl)sulfonyl)phenyl)amino)-4-(phenylthio)butyl)-2,5-diazabicyclo[2.2.1]heptan-2-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 33)

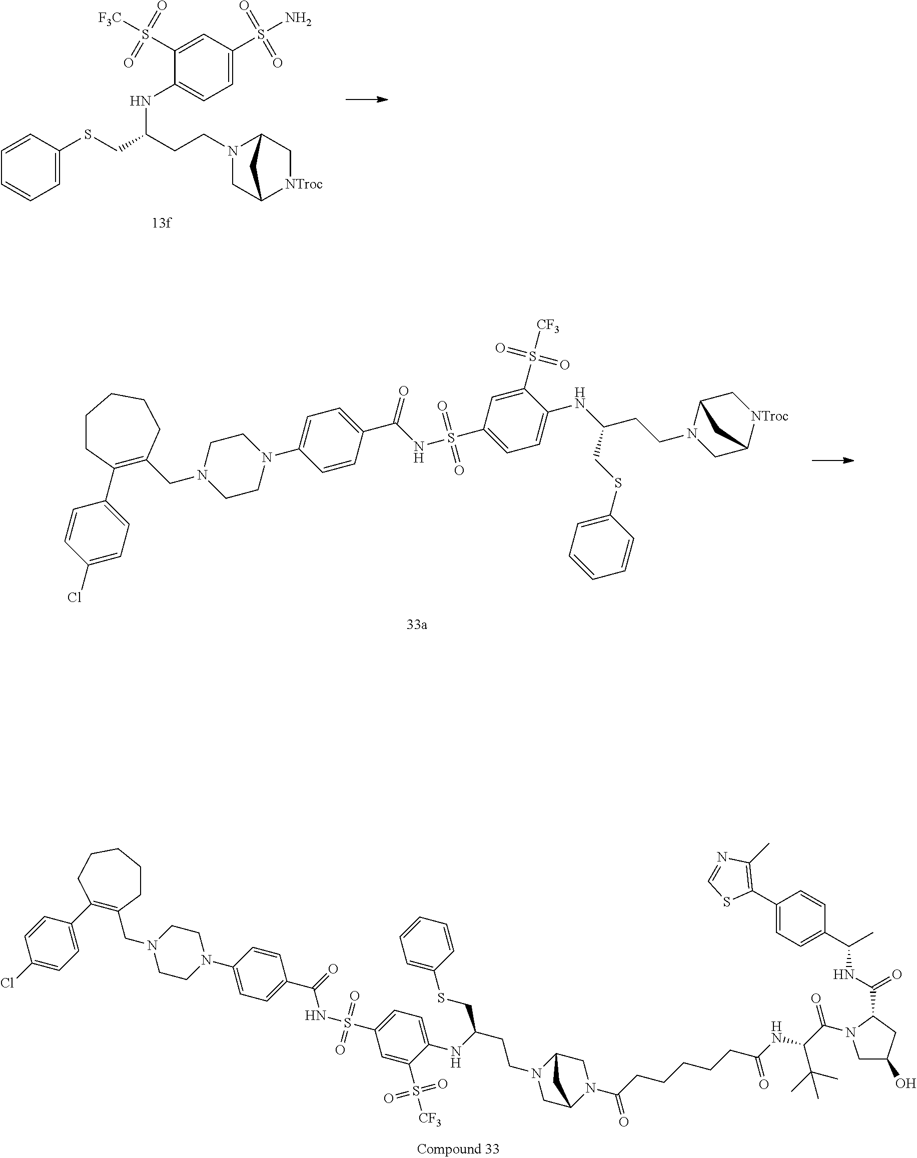

### Step 1: Preparation of 33a

6c (4 g, 8.8 mmol) was dissolved in 100 mL of methanol. Water (10 mL) and sodium hydroxide (1.5 g, 37.5 mmol) were added and the mixture was reacted at 80° C. for 10 h. The reaction solution was cooled to room temperature and concentrated under reduced pressure. The residue was dissolved with 100 mL of water and then extracted with 200 mL of methyl tert-butyl ether to remove the impurities. The aqueous phase was separated, adjusted to pH 6 with 1 mol/L hydrochloric acid, extracted with ethyl acetate (500 mL×2), dried over anhydrous sodium sulfate and concentrated under reduced pressure to afford a crude (2.0 g). 13f (0.60 g, 0.82 mmol) was added to 10 mL of DCM. The above-mentioned crude (0.38 g), DMAP (0.20 g, 1.64 mmol) and EDCI (0.31 g, 1.62 mmol) were sequentially added and the mixture was reacted at 35° C. for 12 h. To the reaction system were added 10 mL of water and 10 mL of DCM. Liquid separation was performed. The aqueous phase was extracted with 5 mL of DCM. The organic phases were combined, dried over anhydrous sodium sulfate and concentrated under reduced pressure and the crude was separated and purified by chromatographic column on silica gel (dichloromethane:methanol (v/v)=10:1) to afford 33a (0.85 g, yield: 90%).

LCMS m/z=1147.1 [M+1]⁺.

### Step 2: Preparation of Compound 33

33a (0.85 g, 0.74 mmol) was added to 8 mL of tetrahydrofuran and 40 mL of methanol. Zinc powder (3.87 g, 59.54 mmol) and ammonium chloride (1.19 g, 22.25 mmol) were sequentially added and the mixture was reacted at 27° C. for 12 h. The reaction system was filtered over diatomaceous earth. The filtrate was concentrated under reduced pressure and the resulting crude was separated and purified by chromatographic column on silica gel (dichloromethane:methanol (v/v)=10:1) to afford a crude (0.46 g). The above-mentioned crude (0.22 g) was added to 10 mL of DCM. Intermediate 1 (0.15 g, 0.26 mmol), triethylamine (0.070 g, 0.69 mmol) and HATU (0.13 g, 0.34 mmol) were sequentially added and the mixture was reacted at room temperature for 2 h. To the reaction solution was added 10 mL of water, followed by liquid separation. The aqueous phase was extracted with DCM (6 mL×2) and the organic phases were combined, dried over anhydrous sodium sulfate and concentrated under reduced pressure. The resulting crude was separated and purified by chromatographic column on silica gel (dichloromethane:methanol (v/v)=10:1) to afford a crude, which was then passed through Pre-HPLC (instrument and preparative column: using SHIMADZU LC-20AP preparative liquid phase chromatographic instrument, preparative column model: Phenomenex C18). Mobile phase system: acetonitrile/water (containing 0.05% NH₄HCO₃). Gradient elution method: gradient elution with acetonitrile from 40% to 70% (elution time: 15 min). Lyophilization was performed to afford compound 33 (165 mg, yield: 41%).

¹H NMR (400 MHz, CDCl₃) δ 8.67 (s, 1H), 8.47-8.35 (m, 1H), 8.06-7.91 (m, 1H), 7.82-7.64 (m, 2H), 7.44-7.08 (m, 13H), 7.03-6.92 (m, 2H), 6.83-6.57 (m, 3H), 6.50-6.31 (m, 1H), 5.15-4.90 (m, 1H), 4.84-3.86 (m, 6H), 3.67-2.94 (m, 10H), 2.94-2.71 (m, 3H), 2.71-1.98 (m, 22H), 1.98-1.42 (m, 14H), 1.40-1.22 (m, 3H), 1.13-0.98 (m, 9H).

LCMS m/z=514.0 [M/3+1]⁺.

Example 34

(2S,4R)-1-((S)-2-(8-((1S,4S)-5-((R)-3-((4-(N-(4-(4-((2-(4-chlorophenyl)cyclohept-1-en-1-yl)methyl)piperazin-1-yl)benzoyl)sulfamoyl)-2-((trifluoromethyl)sulfonyl)phenyl)amino)-4-(phenylthio)butyl)-2,5-diazabicyclo[2.2.1]heptan-2-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (Compound 34)

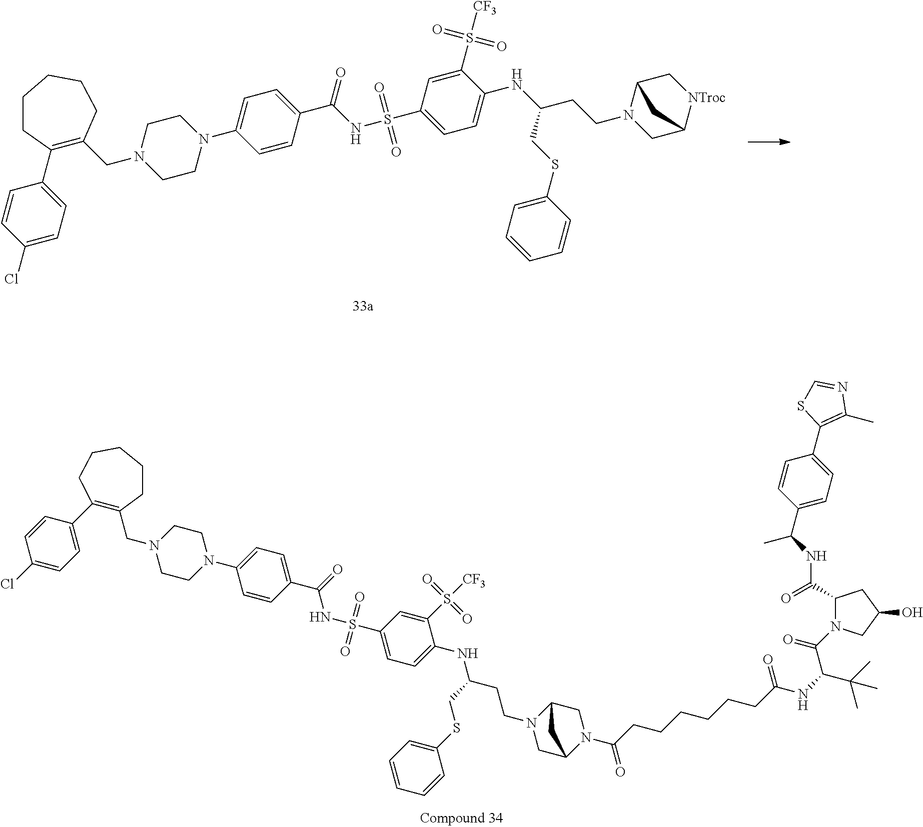

33a (0.85 g, 0.74 mmol) was added to 8 mL of tetrahydrofuran and 40 mL of methanol. Zinc powder (3.87 g, 59.54 mmol) and ammonium chloride (1.19 g, 22.25 mmol) were sequentially added and the mixture was reacted at 27° C. for 12 h. The reaction system was filtered over diatomaceous earth. The filtrate was concentrated under reduced pressure and the resulting crude was separated and purified by chromatographic column on silica gel (dichloromethane:methanol (v/v)=10:1) to afford a crude (0.46 g). The above-mentioned crude (0.22 g) was added to 5 mL of DCM. Intermediate 3 (0.15 g, 0.25 mmol), triethylamine (0.070 g, 0.69 mmol) and HATU (0.13 g, 0.34 mmol) were sequentially added and the mixture was reacted at room temperature for 2 h. To the reaction solution was added 10 mL of water, followed by liquid separation. The aqueous phase was extracted with DCM (6 mL×2). The organic phases were combined, dried over anhydrous sodium sulfate and concentrated under reduced pressure. The resulting crude was separated and purified by chromatographic column on silica gel (dichloromethane:methanol (v/v)=10:1) to afford a crude, which was then passed through Pre-HPLC (instrument and preparative column: using SHIMADZU LC-20AP preparative liquid phase chromatographic instrument, preparative column model: Phenomenex C18, mobile phase system: acetonitrile/water (containing 0.05% NH₄HCO₃), gradient elution method: gradient elution with acetonitrile from 45% to 75% (elution time: 15 min)). Lyophilization was performed to afford compound 34 (158 mg, yield: 41%).

¹H NMR (400 MHz, CDCl₃) δ 8.67 (s, 1H), 8.41-8.35 (m, 1H), 8.08-7.94 (m, 1H), 7.78-7.66 (m, 2H), 7.50-7.16 (m, 13H), 7.01-6.93 (m, 2H), 6.84-6.55 (m, 3H), 6.39-6.25 (m, 1H), 5.14-4.97 (m, 1H), 4.81-3.85 (m, 6H), 3.66-2.73 (m, 13H), 2.73-1.87 (m, 22H), 1.87-1.38 (m, 15H), 1.37-1.20 (m, 4H), 1.13-0.95 (m, 9H).

LCMS m/z=777.5 [M/2+1]⁺.

Example 35

(2S,4R)-1-((S)-2-(7-(4-((R)-3-((4-(N-(4-(4-((2-(4-chloro-2-fluorophenyl)cyclohept-1-en-1-yl)methyl)piperazin-1-yl)benzoyl)sulfamoyl)-2-((trifluoromethyl)sulfonyl)phenyl)amino)-4-(phenylthio)butyl)piperazin-1-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 35)

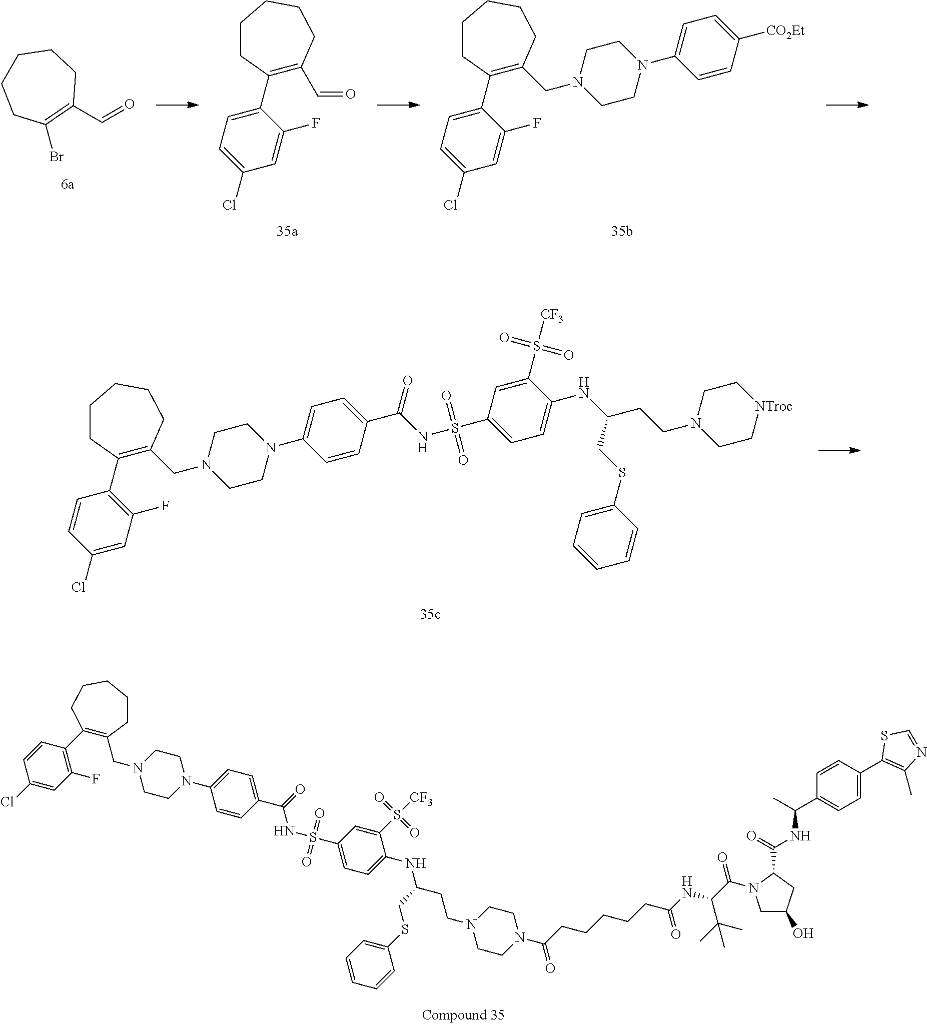

### Step 1: Preparation of 35a

6a (2.20 g, 10.84 mmol) and (4-chloro-2-fluorophenyl)boronic acid (2.83 g, 16.23 mmol) were dissolved in 15 mL of 1,4-dioxane and 1.5 mL of water. Under nitrogen protection, potassium acetate (3.19 g, 32.51 mmol) and Pd(dppf)Cl₂ (0.27 g, 0.36 mmol) were sequentially added and the mixture was reacted at 90° C. for 1 h. The reaction solution was cooled to room temperature. To the reaction solution was slowly added 20 mL of ethyl acetate and 20 mL of water, followed by liquid separation. The aqueous phase was extracted with ethyl acetate (25 mL×2). The organic phases were combined, washed with 25 mL of saturated aqueous sodium chloride solution, dried over anhydrous sodium sulfate and concentrated under reduced pressure and the resulting crude was separated and purified by silica gel column chromatography (petroleum ether/ethyl acetate (v/v)=1:0-20:1) to afford 35a (2.5 g, yield: 92%).

LCMS m/z=253.0 [M+1]⁺.

### Step 2: Preparation of 35b

35a (0.60 g, 2.38 mmol), ethyl 4-(piperazin-1-yl)benzoate (0.83 g, 3.54 mmol) and acetic acid (0.94 g, 15.67 mmol) were added to 50 mL of tetrahydrofuran. Sodium triacetoxyborohydride (1.51 g, 7.13 mmol) was added and the mixture was reacted at room temperature for 16 h. To the reaction solution was added 50 mL of saturated aqueous sodium bicarbonate solution, followed by extraction using ethyl acetate (50 mL×2). The organic phases were combined, washed with 25 mL of saturated aqueous sodium chloride solution, dried over anhydrous sodium sulfate and concentrated under reduced pressure and the resulting crude was separated and purified by silica gel column chromatography (petroleum ether/ethyl acetate (v/v)=1:0-5:1) to afford 35b (0.80 g, yield: 71%).

LCMS m/z=471.2 [M+1]⁺.

### Step 3: Preparation of 35c

35b (0.80 g, 1.70 mmol) was dissolved in mixed solvents of 8 mL of tetrahydrofuran, 4 mL of ethanol and 2 mL of water. Sodium hydroxide (0.27 g, 6.75 mmol) was added and the mixture was stirred at 65° C. for 16 h. The reaction solution was cooled to room temperature and concentrated under reduced pressure. To the residue was added 20 mL of water. Then the mixture was extracted with 20 mL of methyl tert-butyl ether to remove the impurities. The aqueous phase was adjusted to pH 5 with 1 mol/L hydrochloric acid and extracted with ethyl acetate (15 mL×2). The organic phases were combined, washed with 25 mL of saturated sodium chloride solution, dried over anhydrous sodium sulfate and concentrated under reduced pressure to afford a crude (0.68 g). Intermediate 2 (0.60 g, 0.82 mmol) was added to 12 mL of DCM. The above-mentioned crude (0.40 g), DMAP (0.20 g, 1.64 mmol) and EDCI (0.31 g, 1.62 mmol) were sequentially added and the mixture was warmed to 35° C. and reacted for 12 h. To the reaction system were added 5 mL of water and 3 mL of DCM. Liquid separation was performed. The aqueous phase was extracted with 5 mL of DCM. The organic phases were combined, dried over anhydrous sodium sulfate and concentrated under reduced pressure and the crude was separated and purified by chromatographic column on silica gel (dichloromethane:methanol (v/v)=50:1-10:1) to afford 35c (0.62 g, yield: 66%).

LCMS m/z=1153.1 [M+1]⁺.

### Step 4: Preparation of Compound 35

35c (0.62 g, 0.54 mmol) was added to 5 mL of tetrahydrofuran and 30 mL of methanol. Zinc powder (2.83 g, 43.54 mmol) and ammonium chloride (0.87 g, 16.26 mmol) were sequentially added and the mixture was reacted at 27° C. for 12 h. The reaction solution was filtered over diatomaceous earth. The filtrate was concentrated under reduced pressure and the resulting crude was separated and purified by chromatographic column on silica gel (dichloromethane:methanol (v/v)=10:1) to afford a crude (0.34 g). The above-mentioned crude (0.17 g) was added to 15 mL of DCM. Intermediate 1 (0.11 g, 0.19 mmol), triethylamine (0.17 g, 1.70 mmol) and HATU (0.097 g, 0.26 mmol) were sequentially added and the mixture was reacted at room temperature for 2 h. To the reaction solution was added 5 mL of water, followed by liquid separation. The aqueous phase was extracted with DCM (6 mL×2) and the organic phases were combined, dried over anhydrous sodium sulfate and concentrated under reduced pressure. The resulting crude was separated and purified by chromatographic column on silica gel (dichloromethane:methanol (v/v)=50:1-10:1) to afford a crude, which was then passed through Pre-HPLC (instrument and preparative column: using SHIMADZU LC-20AP preparative liquid phase chromatographic instrument, preparative column model: Phenomenex C18). Mobile phase system: acetonitrile/water (containing 0.05% NH₄HCO₃). Gradient elution method: gradient elution with acetonitrile from 40% to 70% (elution time: 15 min). Lyophilization was performed to afford compound 35 (24.02 mg, yield: 8%).

¹H NMR (400 MHz, CDCl₃) δ 8.67 (s, 1H), 8.42-8.30 (m, 1H), 8.14-8.05 (m, 1H), 7.74-7.62 (m, 2H), 7.45-7.21 (m, 10H), 7.16-6.88 (m, 4H), 6.82-6.70 (m, 2H), 6.68-6.57 (m, 1H), 6.36-6.21 (m, 1H), 5.15-5.00 (m, 1H), 4.80-4.67 (m, 1H), 4.65-4.55 (m, 1H), 4.55-4.44 (m, 1H), 4.17-4.05 (m, 1H), 4.00-3.83 (m, 1H), 3.75-3.52 (m, 2H), 3.50-3.20 (m, 7H), 3.18-2.95 (m, 2H), 2.95-2.70 (m, 2H), 2.60-1.95 (m, 24H), 1.90-1.40 (m, 14H), 1.40-1.20 (m, 2H), 1.04 (s, 9H).

LCMS m/z=516.1 [M/3+1]⁺.

Example 36

(2S,4R)-1-((S)-2-(8-(4-((R)-3-((4-(N-(4-(4-((2-(4-chloro-2-fluorophenyl)cyclohept-1-en-1-yl)methyl)piperazin-1-yl)benzoyl)sulfamoyl)-2-((trifluoromethyl)sulfonyl)phenyl)amino)-4-(phenylthio)butyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (Compound 36)

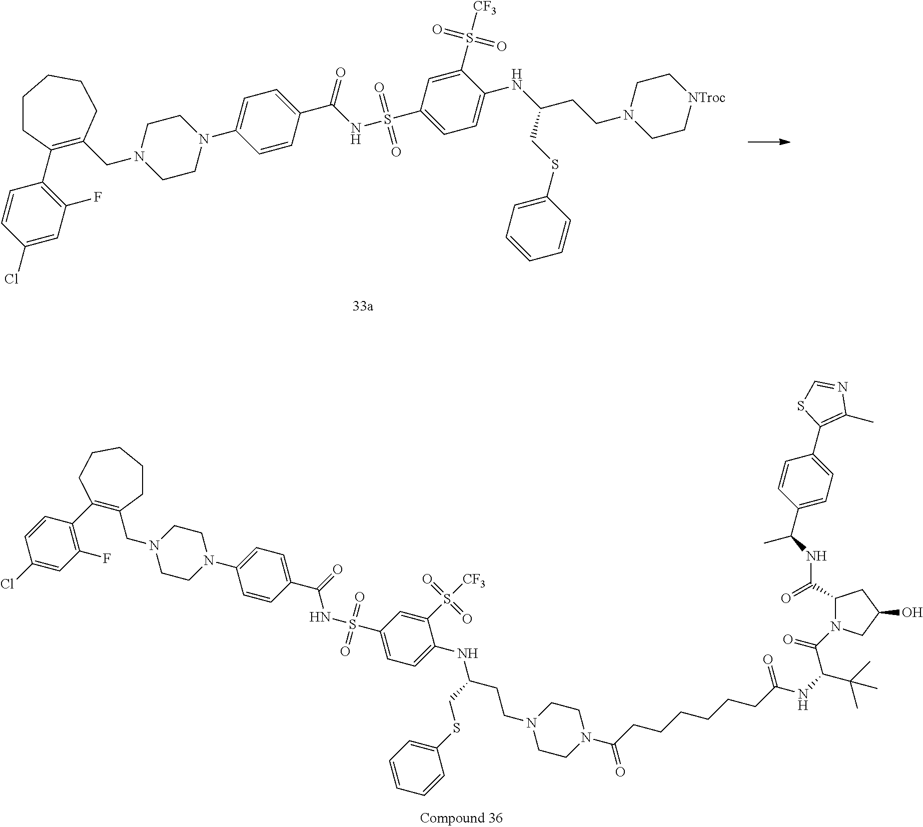

35c (0.62 g, 0.54 mmol) was added to 5 mL of tetrahydrofuran and 30 mL of methanol. Zinc powder (2.83 g, 43.54 mmol) and ammonium chloride (0.87 g, 16.26 mmol) were sequentially added and the mixture was reacted at 27° C. for 12 h. The reaction solution was filtered over diatomaceous earth. The filtrate was concentrated under reduced pressure and the resulting crude was separated and purified by chromatographic column on silica gel (dichloromethane:methanol (v/v)=10:1) to afford a crude (0.34 g). The above-mentioned crude (0.17 g) was added to 5 mL of DCM. Intermediate 3 (0.11 g, 0.18 mmol), triethylamine (0.17 g, 1.70 mmol) and HATU (0.097 g, 0.26 mmol) were sequentially added and the mixture was reacted at room temperature for 2 h. To the reaction solution was added 10 mL of water, followed by extraction using DCM (6 mL×2). The organic phases were combined, dried over anhydrous sodium sulfate and concentrated under reduced pressure. The resulting crude was separated and purified by chromatographic column on silica gel (dichloromethane:methanol (v/v)=50:1-10:1) to afford a crude, which was then passed through Pre-HPLC (instrument and preparative column: using SHIMADZU LC-20AP preparative liquid phase chromatographic instrument, preparative column model: Phenomenex C18). Mobile phase system: acetonitrile/water (containing 0.05% NH₄HCO₃). Gradient elution method: gradient elution with acetonitrile from 42% to 72% (elution time: 15 min). Lyophilization was performed to afford compound 36 (25 mg, yield: 9%).

¹H NMR (400 MHz, CDCl₃) δ 8.67 (s, 1H), 8.40-8.32 (m, 1H), 8.13-8.05 (m, 1H), 7.75-7.61 (m, 2H), 7.45-7.21 (m, 10H), 7.14-7.02 (m, 3H), 6.98-6.90 (m, 1H), 6.84-6.58 (m, 3H), 6.32-6.15 (m, 1H), 5.15-5.00 (m, 1H), 4.78-4.68 (m, 1H), 4.64-4.46 (m, 2H), 4.17-4.06 (m, 1H), 4.02-3.84 (m, 1H), 3.80-3.20 (m, 9H), 3.18-2.72 (m, 4H), 2.70-2.00 (m, 24H), 1.95-1.20 (m, 18H), 1.04 (s, 9H).

LCMS m/z=520.8 [M/3+1]⁺.

Example 37

(2S,4R)-1-((S)-2-(7-((1S,4S)-5-((R)-3-((4-(N-(4-(4-((2-(4-chloro-2-fluorophenyl)cyclohept-1-en-1-yl)methyl)piperazin-1-yl)benzoyl)sulfamoyl)-2-((trifluoromethyl)sulfonyl)phenyl)amino)-4-(phenylthio)butyl)-2,5-diazabicyclo[2.2.1]heptan-2-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 37)

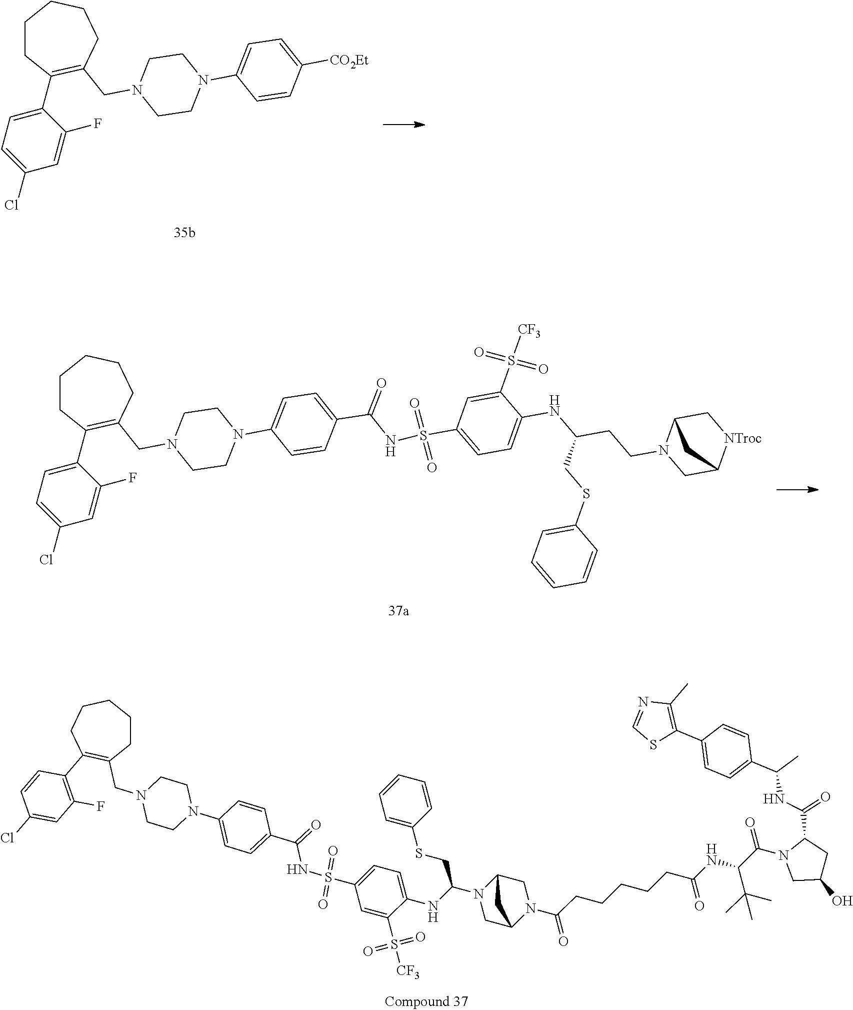

### Step 1: Preparation of 37a

35b (0.80 g, 1.70 mmol) was dissolved in mixed solvents of 8 mL of tetrahydrofuran, 4 mL of ethanol and 2 mL of water. Sodium hydroxide (0.27 g, 6.75 mmol) was added and the mixture was stirred at 65° C. for 16 h. The reaction solution was cooled to room temperature and concentrated under reduced pressure. To the residue was added 20 mL of water. Then the mixture was extracted with 20 mL of methyl tert-butyl ether to remove the impurities. The aqueous phase was adjusted to pH 5 with 1 mol/L hydrochloric acid and extracted with ethyl acetate (15 mL×2). The organic phases were combined, washed with 25 mL of saturated sodium chloride solution, dried over anhydrous sodium sulfate and concentrated under reduced pressure to afford a crude (0.68 g). 13f (0.60 g, 0.82 mmol) was added to 12 mL of DCM. The above-mentioned crude (0.39 g), DMAP (0.20 g, 1.64 mmol) and EDCI (0.31 g, 1.62 mmol) were sequentially added and the mixture was reacted at 35° C. for 12 h. To the reaction system were added 5 mL of water and 3 mL of DCM. Liquid separation was performed. The aqueous phase was extracted with 5 mL of DCM. The organic phases were combined, dried over anhydrous sodium sulfate and concentrated under reduced pressure and the crude was separated and purified by chromatographic column on silica gel (dichloromethane:methanol (v/v)=50:1-10:1) to afford 37a (0.75 g, yield: 79%).

LCMS m/z=1165.2 [M+1]⁺.

### Step 2: Preparation of Compound 37

37a (0.75 g, 0.64 mmol) was added to 8 mL of tetrahydrofuran and 40 mL of methanol. Zinc powder (3.35 g, 51.54 mmol) and ammonium chloride (1.03 g, 19.26 mmol) were sequentially added and the mixture was reacted at 27° C. for 12 h. The reaction solution was filtered over diatomaceous earth. The filtrate was concentrated under reduced pressure and the resulting crude was separated and purified by chromatographic column on silica gel (dichloromethane:methanol (v/v)=50:1-10:1) to afford a crude (0.46 g). The above-mentioned crude (0.23 g) was added to 10 mL of DCM. Intermediate 1 (0.15 g, 0.26 mmol), triethylamine (0.23 g, 2.30 mmol) and HATU (0.13 g, 0.34 mmol) were sequentially added and the mixture was reacted at room temperature for 2 h. To the reaction solution was added 10 mL of water, followed by liquid separation. The aqueous phase was extracted with DCM (6 mL×2) and the organic phases were combined, dried over anhydrous sodium sulfate and concentrated under reduced pressure. The resulting crude was separated and purified by chromatographic column on silica gel (dichloromethane:methanol (v/v)=50:1-10:1) to afford a crude, which was then passed through Pre-HPLC (instrument and preparative column: using SHIMADZU LC-20AP preparative liquid phase chromatographic instrument, preparative column model: Phenomenex C18). Mobile phase system: acetonitrile/water (containing 10 mmol/L NH₄HCO₃). Gradient elution method: gradient elution with acetonitrile from 53% to 83% (elution time: 10 min). Lyophilization was performed to afford compound 37 (68 mg, yield: 17%).

¹H NMR (400 MHz, CDCl₃) δ 8.67 (s, 1H), 8.45-8.35 (m, 1H), 8.08-7.92 (m, 1H), 7.80-7.65 (m, 2H), 7.45-7.10 (m, 11H), 7.09-7.01 (m, 2H), 6.98-6.87 (m, 1H), 6.82-6.58 (m, 3H), 6.51-6.32 (m, 1H), 5.14-4.92 (m, 1H), 4.84-3.85 (m, 6H), 3.68-2.92 (m, 10H), 2.87-1.95 (m, 25H), 1.92-1.42 (m, 14H), 1.40-1.21 (m, 3H), 1.09-0.99 (m, 9H).

LCMS m/z=520.2 [M/3+1]⁺.

Example 38

(2S,4R)-1-((S)-2-(8-((1S,4S)-5-((R)-3-((4-(N-(4-(4-((2-(4-chloro-2-fluorophenyl)cyclohept-1-en-1-yl)methyl)piperazin-1-yl)benzoyl)sulfamoyl)-2-((trifluoromethyl)sulfonyl)phenyl)amino)-4-(phenylthio)butyl)-2,5-diazabicyclo[2.2.1]heptan-2-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (Compound 38)

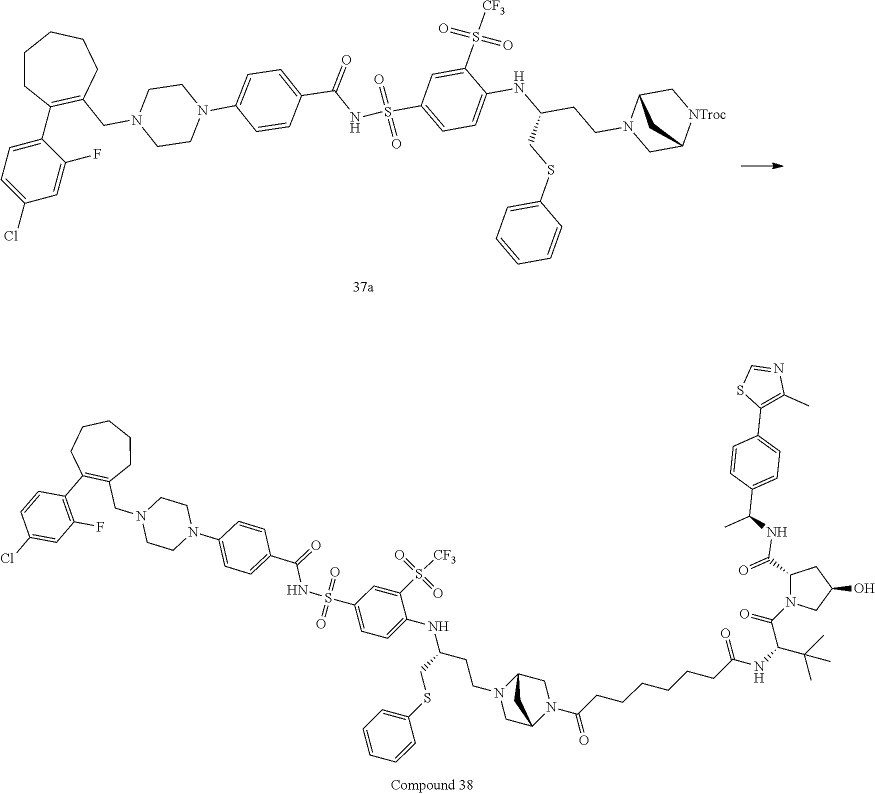

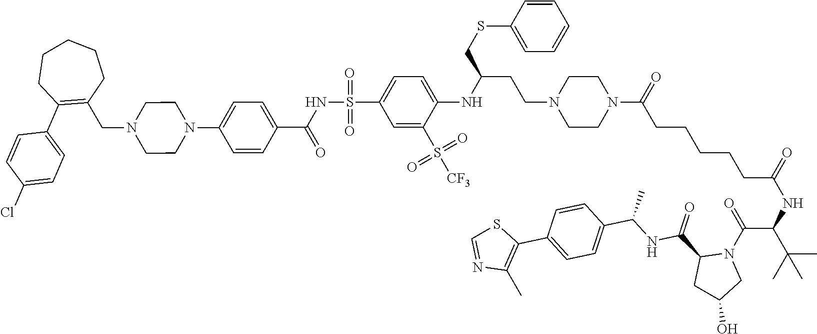 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCCCC6)CC5)cc4)cc3S(=O)(=O)C(F)(F)F)CC2)C(C)(C)C)cc1]

37a (0.75 g, 0.64 mmol) was added to 8 mL of tetrahydrofuran and 40 mL of methanol. Zinc powder (3.35 g, 51.54 mmol) and solid ammonium chloride (1.03 g, 19.26 mmol) were sequentially added and the mixture was reacted at 27° C. for 12 h. The reaction solution was filtered over diatomaceous earth. The filtrate was concentrated under reduced pressure and the resulting crude was separated and purified by chromatographic column on silica gel (dichloromethane:methanol (v/v)=50:1-10:1) to afford a crude (0.46 g). The above-mentioned crude (0.23 g) was added to 10 mL of DCM. Intermediate 3 (0.15 g, 0.25 mmol), triethylamine (0.070 g, 0.69 mmol) and HATU (0.13 g, 0.34 mmol) were sequentially added and the mixture was reacted at room temperature for 2 h. To the reaction solution was added 10 mL of water, followed by liquid separation. The aqueous phase was extracted with DCM (6 mL×2). The organic phases were combined, dried over anhydrous sodium sulfate and concentrated under reduced pressure. The resulting crude was separated and purified by chromatographic column on silica gel (dichloromethane:methanol (v/v)=50:1-10:1) to afford a crude, which was then passed through Pre-HPLC (instrument and preparative column: using SHIMADZU LC-20AP preparative liquid phase chromatographic instrument, preparative column model: Phenomenex C18, mobile phase system: acetonitrile/water (containing 10 mmol/L NH₄HCO₃), gradient elution method: gradient elution with acetonitrile from 53% to 83% (elution time: 10 min)). Lyophilization was performed to afford compound 38 (66 mg, yield: 17%).

¹H NMR (400 MHz, CDCl₃) δ 8.67 (s, 1H), 8.42-8.35 (m, 1H), 8.07-7.94 (m, 1H), 7.78-7.66 (m, 2H), 7.50-7.14 (m, 11H), 7.11-7.00 (m, 2H), 6.98-6.88 (m, 1H), 6.83-6.57 (m, 3H), 6.35-6.23 (m, 1H), 5.15-4.97 (m, 1H), 4.83-3.88 (m, 6H), 3.65-2.95 (m, 10H), 2.95-2.03 (m, 24H), 2.01-1.38 (m, 16H), 1.35-1.20 (m, 4H), 1.10-0.98 (m, 9H).

LCMS m/z=524.8 [M/3+1]⁺.

Example 39

cis-(2S,4R)-1-((S)-2-(7-(5-((R)-3-((4-(N-(4-(4-((2-(4-chloro-2-fluorophenyl)cyclohept-1-en-1-yl)methyl)piperazin-1-yl)benzoyl)sulfamoyl)-2-((trifluoromethyl)sulfonyl)phenyl)amino)-4-(phenylthio)butyl)hexahydropyrrolo[3,4-c]pyrrol-2(1H)-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 39)

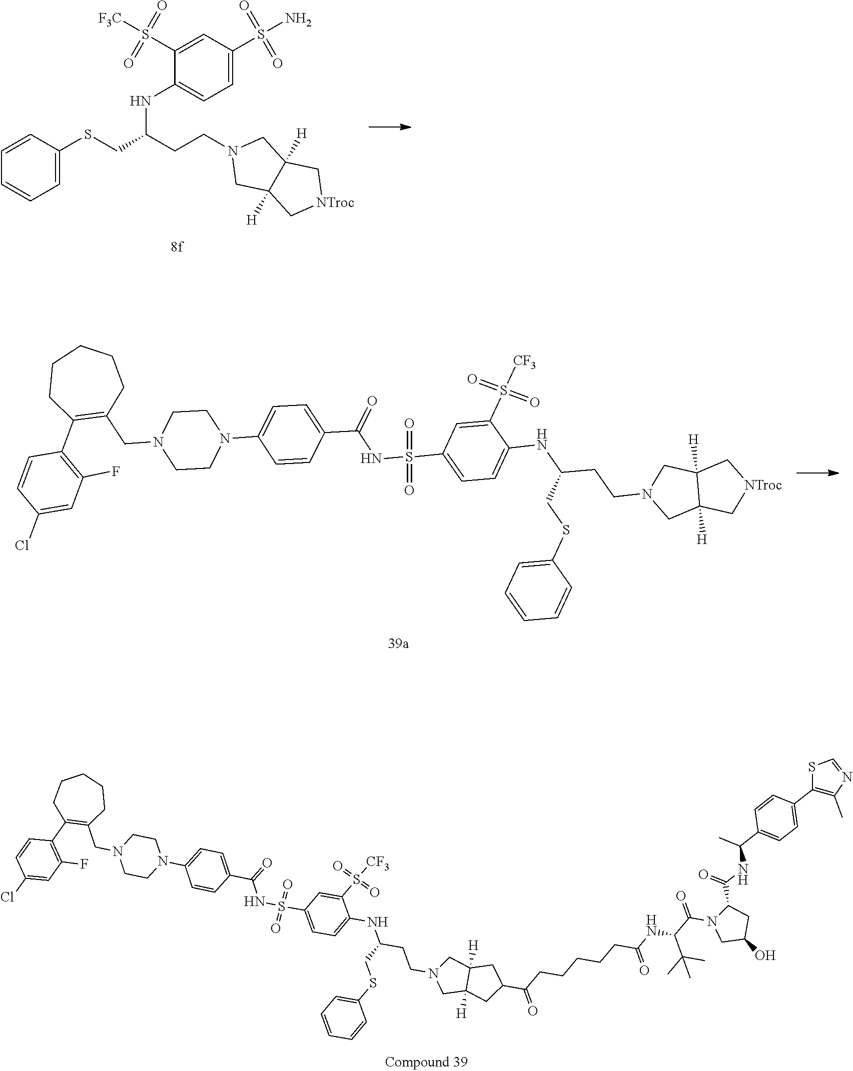

### Step 1: Preparation of 39a

35b (0.80 g, 1.70 mmol) was dissolved in mixed solvents of 8 mL of tetrahydrofuran, 4 mL of ethanol and 2 mL of water. Sodium hydroxide (0.27 g, 6.75 mmol) was added and the mixture was stirred at 65° C. for 16 h. The reaction solution was cooled to room temperature and concentrated under reduced pressure. To the residue was added 20 mL of water. Then the mixture was extracted with 20 mL of methyl tert-butyl ether to remove the impurities. The aqueous phase was adjusted to pH 5 with 1 mol/L hydrochloric acid and extracted with ethyl acetate (15 mL×2). The organic phases were combined, washed with 25 mL of saturated sodium chloride solution, dried over anhydrous sodium sulfate and concentrated under reduced pressure to afford a crude (0.68 g). 8f (1.00 g, 1.33 mmol) was added to 12 mL of DCM. The above-mentioned crude (0.65 g), DMAP (0.32 g, 2.62 mmol) and EDCI (0.51 g, 2.67 mmol) were sequentially added and the mixture was reacted at 35° C. for 12 h. To the reaction system were added 10 mL of water and 5 mL of DCM. Liquid separation was performed. The aqueous phase was extracted with 5 mL of DCM. The organic phases were combined, dried over anhydrous sodium sulfate and concentrated under reduced pressure and the crude was separated and purified by chromatographic column on silica gel (dichloromethane:methanol (v/v)=20:1) to afford 39a (0.90 g, yield: 57%).

LCMS m/z=1179.1 [M+1]⁺.

### Step 2: Preparation of Compound 39

39a (0.90 g, 0.76 mmol) was added to 9 mL of tetrahydrofuran and 45 mL of methanol. Zinc powder (3.98 g, 61.23 mmol) and ammonium chloride (1.22 g, 22.81 mmol) were sequentially added and the mixture was reacted at 27° C. for 12 h. The reaction solution was filtered over diatomaceous earth. The filtrate was concentrated under reduced pressure and the resulting crude was separated and purified by chromatographic column on silica gel (dichloromethane:methanol (v/v)=10:1) to afford a crude (0.70 g). The above-mentioned crude (0.35 g) was added to 15 mL of DCM. Intermediate 1 (0.23 g, 0.40 mmol), triethylamine (0.11 g, 1.09 mmol) and HATU (0.20 g, 0.526 mmol) were sequentially added and the mixture was reacted at room temperature for 2 h. To the reaction solution was added 10 mL of water, followed by liquid separation. The aqueous phase was extracted with DCM (6 mL×2) and the organic phases were combined, dried over anhydrous sodium sulfate and concentrated under reduced pressure. The resulting crude was separated and purified by chromatographic column on silica gel (dichloromethane:methanol (v/v)=10:1) to afford a crude, which was then passed through Pre-HPLC (instrument and preparative column: using SHIMADZU LC-20AP preparative liquid phase chromatographic instrument, preparative column model: Phenomenex C18). Mobile phase system: acetonitrile/water (containing 10 mmol/L NH₄HCO₃). Gradient elution method: gradient elution with acetonitrile from 50% to 70% (elution time: 20 min). Lyophilization was performed to afford compound 39 (280 mg, yield: 45%).

¹H NMR (400 MHz, CDCl₃) δ 8.69-8.64 (m, 1H), 8.53-8.46 (m, 1H), 7.96-7.70 (m, 3H), 7.45-7.16 (m, 9H), 7.12-6.87 (m, 5H), 6.85-6.65 (m, 3H), 6.52-6.31 (m, 1H), 5.15-4.88 (m, 1H), 4.86-4.65 (m, 2H), 4.57-4.45 (m, 1H), 4.25-3.86 (m, 2H), 3.78-3.41 (m, 3H), 3.40-2.94 (m, 8H), 2.94-1.98 (m, 26H), 1.92-1.40 (m, 14H), 1.39-1.22 (m, 4H), 1.13-0.98 (m, 9H).

LCMS m/z=786.6 [M/2+1]⁺.

Example 40

cis-(2S,4R)-1-((S)-2-(8-(5-((R)-3-((4-(N-(4-(4-((2-(4-chloro-2-fluorophenyl)cyclohept-1-en-1-yl)methyl)piperazin-1-yl)benzoyl)sulfamoyl)-2-((trifluoromethyl)sulfonyl)phenyl)amino)-4-(phenylthio)butyl)hexahydropyrrolo[3,4-c]pyrrol-2(1H)-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (Compound 40)

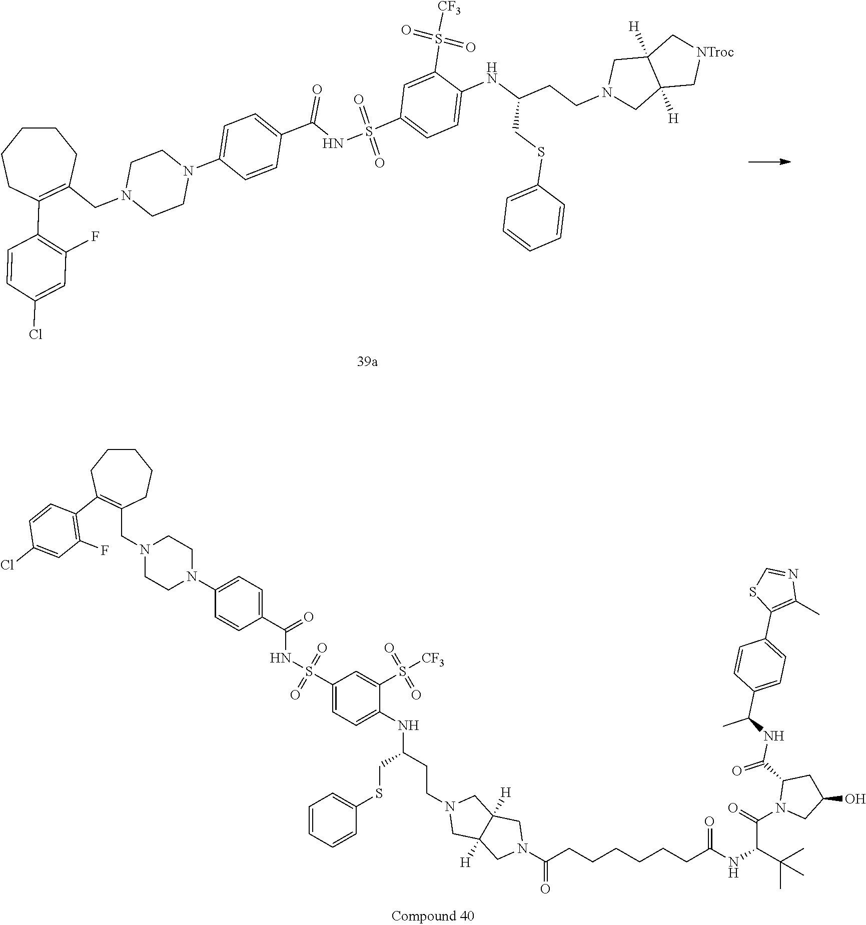

39a (0.90 g, 0.76 mmol) was added to 9 mL of tetrahydrofuran and 45 mL of methanol. Zinc powder (3.98 g, 61.23 mmol) and ammonium chloride (1.22 g, 22.81 mmol) were sequentially added and the mixture was reacted at 27° C. for 12 h. The reaction solution was filtered over diatomaceous earth. The filtrate was concentrated under reduced pressure and the resulting crude was separated and purified by chromatographic column on silica gel (dichloromethane:methanol (v/v)=10:1) to afford a crude (0.70 g). The above-mentioned crude (0.35 g) was added to 15 mL of DCM. Intermediate 3 (0.23 g, 0.38 mmol), triethylamine (0.11 g, 1.09 mmol) and HATU (0.20 g, 0.526 mmol) were sequentially added and the mixture was reacted at room temperature for 2 h. To the reaction solution was added 10 mL of water, followed by liquid separation. The aqueous phase was extracted with DCM (6 mL×2). The organic phases were combined, dried over anhydrous sodium sulfate and concentrated under reduced pressure. The resulting crude was separated and purified by chromatographic column on silica gel (dichloromethane:methanol (v/v)=10:1) to afford a crude, which was then passed through Pre-HPLC (instrument and preparative column: using SHIMADZU LC-20AP preparative liquid phase chromatographic instrument, preparative column model: Phenomenex C18, mobile phase system: acetonitrile/water (containing 10 mmol/L NH₄HCO₃), gradient elution method: gradient elution with acetonitrile from 50% to 70% (elution time: 20 min)). Lyophilization was performed to afford compound 40 (290 mg, yield: 48%).

¹H NMR (400 MHz, CDCl₃) δ 8.67 (s, 1H), 8.52-8.38 (m, 1H), 8.04-7.87 (m, 1H), 7.84-7.72 (m, 2H), 7.56-7.14 (m, 10H), 7.09-6.89 (m, 4H), 6.84-6.73 (m, 2H), 6.72-6.62 (m, 1H), 6.50-6.24 (m, 1H), 5.15-4.93 (m, 1H), 4.84-4.62 (m, 2H), 4.57-4.45 (m, 1H), 4.18-3.85 (m, 2H), 3.69-3.42 (m, 3H), 3.39-2.01 (m, 34H), 1.90-1.16 (m, 20H), 1.13-0.96 (m, 9H).

LCMS m/z=793.9 [M/2+1]⁺.

Example 41

cis-(2S,4R)-1-((S)-2-(7-(5-((R)-3-((4-(N-(4-(4-((2-(4-chlorophenyl)cyclohept-1-en-1-yl)methyl)piperazin-1-yl)benzoyl)sulfamoyl)-2-((trifluoromethyl)sulfonyl)phenyl)amino)-4-(phenylthio)butyl)hexahydropyrrolo[3,4-c]pyrrol-2(1H)-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 41)

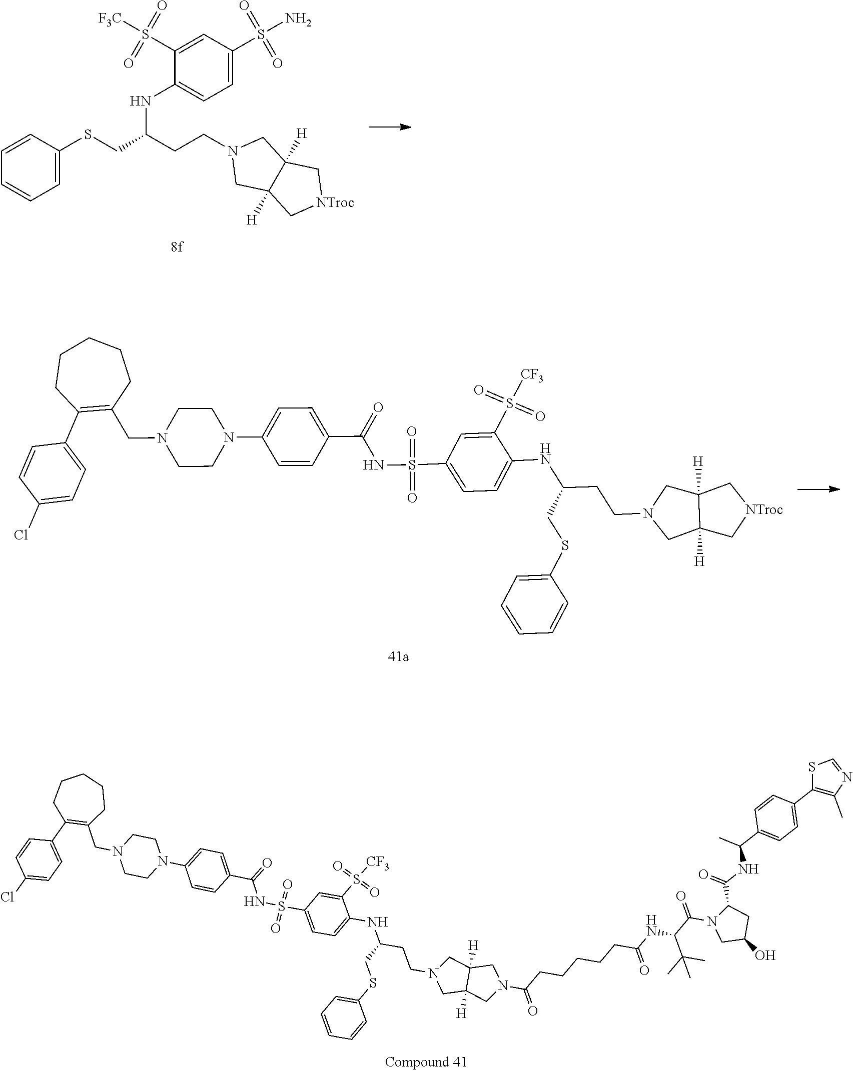

### Step 1: Preparation of 41a

6c (0.40 g, 0.88 mmol) was dissolved in 10 mL of methanol. Water (1 mL) and sodium hydroxide (0.15 g, 3.75 mmol) were added and the mixture was reacted at 80° C. for 10 h. The reaction solution was cooled to room temperature and concentrated under reduced pressure. The residue was dissolved with 10 mL of water and then extracted with 20 mL of methyl tert-butyl ether to remove the impurities. The aqueous phase was separated, adjusted to pH 6 with 1 mol/L hydrochloric acid, extracted with ethyl acetate (100 mL×2), dried over anhydrous sodium sulfate and concentrated under reduced pressure to afford a crude (0.20 g). 8f (1.00 g, 1.33 mmol) was added to 12 mL of DCM. The above-mentioned crude (0.62 g), DMAP (0.32 g, 2.62 mmol) and EDCI (0.51 g, 2.67 mmol) were sequentially added and the mixture was reacted at 35° C. for 12 h. To the reaction system were added 10 mL of water and 5 mL of dichloromethane. Liquid separation was performed. The aqueous phase was extracted with 5 mL of DCM. The organic phases were combined, dried over anhydrous sodium sulfate and concentrated under reduced pressure and the crude was separated and purified by chromatographic column on silica gel (dichloromethane:methanol (v/v)=20:1) to afford 41a (1.00 g, yield: 65%).

LCMS m/z=1161.2 [M+1]⁺.

### Step 2: Preparation of Compound 41

41a (1.00 g, 0.86 mmol) was added to 10 mL of tetrahydrofuran and 50 mL of methanol. Zinc powder (4.50 g, 69.23 mmol) and ammonium chloride (1.38 g, 25.80 mmol) were sequentially added and the mixture was reacted at 27° C. for 12 h. The reaction solution was filtered over diatomaceous earth. The filtrate was concentrated under reduced pressure and the resulting crude was separated and purified by chromatographic column on silica gel (dichloromethane:methanol (v/v)=10:1) to afford a crude (0.80 g). The above-mentioned crude (0.40 g) was added to 15 mL of DCM. Intermediate 1 (0.26 g, 0.45 mmol), triethylamine (0.12 g, 1.19 mmol) and HATU (0.23 g, 0.605 mmol) were sequentially added and the mixture was reacted at room temperature for 2 h. To the reaction solution was added 10 mL of water, followed by liquid separation. The aqueous phase was extracted with DCM (6 mL×2) and the organic phases were combined, dried over anhydrous sodium sulfate and concentrated under reduced pressure. The resulting crude was separated and purified by chromatographic column on silica gel (dichloromethane:methanol (v/v)=10:1) to afford a crude, which was then passed through Pre-HPLC (instrument and preparative column: using SHIMADZU LC-20AP preparative liquid phase chromatographic instrument, preparative column model: Phenomenex C18). Mobile phase system: acetonitrile/water (containing 10 mmol/L NH₄HCO₃). Gradient elution method: gradient elution with acetonitrile from 45% to 75% (elution time: 20 min). Lyophilization was performed to afford compound 41 (150 mg, yield: 21%).

¹H NMR (400 MHz, CDCl₃) δ 8.70-8.62 (m, 1H), 8.52-8.44 (m, 1H), 7.96-7.70 (m, 3H), 7.44-6.92 (m, 15H), 6.83-6.65 (m, 3H), 6.55-6.35 (m, 1H), 5.15-4.90 (m, 1H), 4.85-4.65 (m, 2H), 4.55-4.45 (m, 1H), 4.22-3.86 (m, 2H), 3.80-3.40 (m, 3H), 3.37-1.96 (m, 34H), 1.90-1.20 (m, 18H), 1.10-0.98 (m, 9H). LCMS m/z=777.3 [M/2+1]⁺.

Example 42

cis-(2S,4R)-1-((S)-2-(8-(5-((R)-3-((4-(N-(4-(4-((2-(4-chlorophenyl)cyclohept-1-en-1-yl)methyl)piperazin-1-yl)benzoyl)sulfamoyl)-2-((trifluoromethyl)sulfonyl)phenyl)amino)-4-(phenylthio)butyl)hexahydropyrrolo[3,4-c]pyrrol-2(1H)-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (Compound 42)

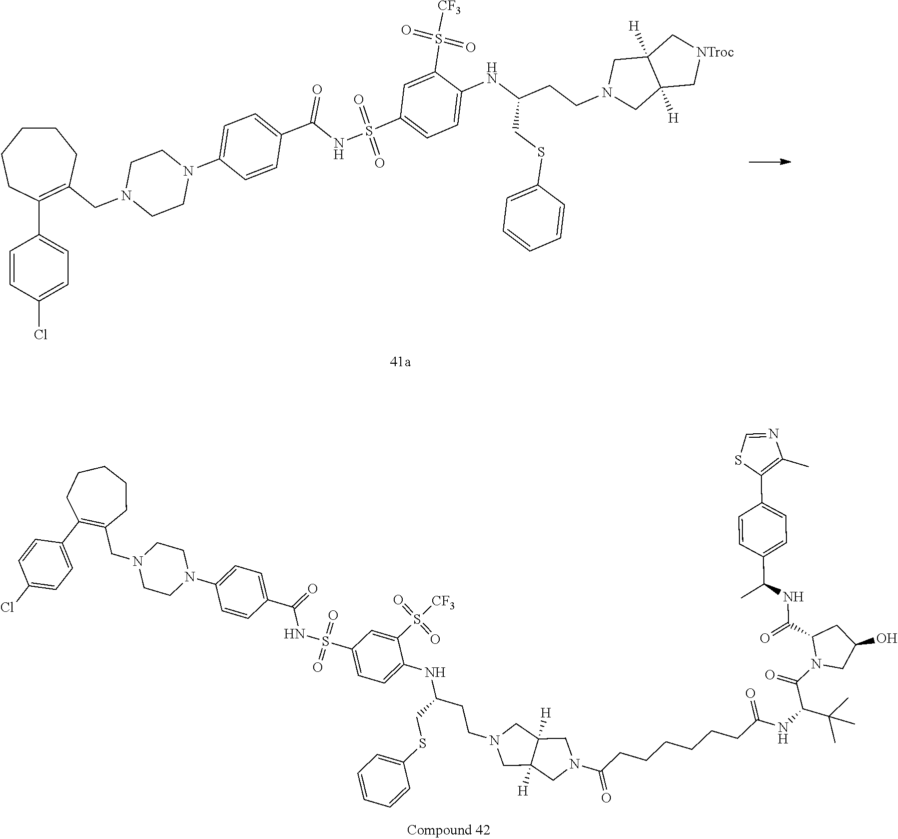

41a (1.00 g, 0.86 mmol) was added to 10 mL of tetrahydrofuran and 50 mL of methanol. Zinc powder (4.50 g, 69.23 mmol) and ammonium chloride (1.38 g, 25.80 mmol) were sequentially added and the mixture was reacted at 27° C. for 12 h. The reaction solution was filtered over diatomaceous earth. The filtrate was concentrated under reduced pressure and the resulting crude was separated and purified by chromatographic column on silica gel (dichloromethane:methanol (v/v)=10:1) to afford a crude (0.80 g). The above-mentioned crude (0.40 g) was added to 15 mL of DCM. Intermediate 3 (0.27 g, 0.45 mmol), triethylamine (0.12 g, 1.19 mmol) and HATU (0.23 g, 0.605 mmol) were sequentially added and the mixture was reacted at room temperature for 2 h. To the reaction solution was added 10 mL of water, followed by liquid separation. The aqueous phase was extracted with DCM (6 mL×2). The organic phases were combined, dried over anhydrous sodium sulfate and concentrated under reduced pressure. The resulting crude was separated and purified by chromatographic column on silica gel (dichloromethane:methanol (v/v)=10:1) to afford a crude, which was then passed through Pre-HPLC (instrument and preparative column: using SHIMADZU LC-20AP preparative liquid phase chromatographic instrument, preparative column model: Phenomenex C18, mobile phase system: acetonitrile/water (containing 10 mmol/L NH₄HCO₃), gradient elution method: gradient elution with acetonitrile from 45% to 75% (elution time: 20 min)). Lyophilization was performed to afford compound 42 (270 mg, yield: 38%).

¹H NMR (400 MHz, CDCl₃) δ 8.69-8.63 (m, 1H), 8.49-8.39 (m, 1H), 8.04-7.88 (m, 1H), 7.84-7.70 (m, 2H), 7.57-7.14 (m, 12H), 7.06-6.94 (m, 3H), 6.83-6.59 (m, 3H), 6.54-6.32 (m, 1H), 5.14-4.92 (m, 1H), 4.80-4.61 (m, 2H), 4.55-4.45 (m, 1H), 4.16-3.84 (m, 2H), 3.70-3.41 (m, 3H), 3.33-1.99 (m, 34H), 1.91-1.15 (m, 20H), 1.14-0.94 (m, 9H). LCMS m/z=784.5 [M/2+1]⁺.

Example 43

(2S,4R)-1-((S)-2-(7-(4-((R)-3-((4-(N-(4-(4-((2-(4-chlorophenyl)cyclooct-1-en-1-yl)methyl)piperazin-1-yl)benzoyl)sulfamoyl)-2-((trifluoromethyl)sulfonyl)phenyl)amino)-4-(phenylthio)butyl)piperazin-1-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (Compound 43)

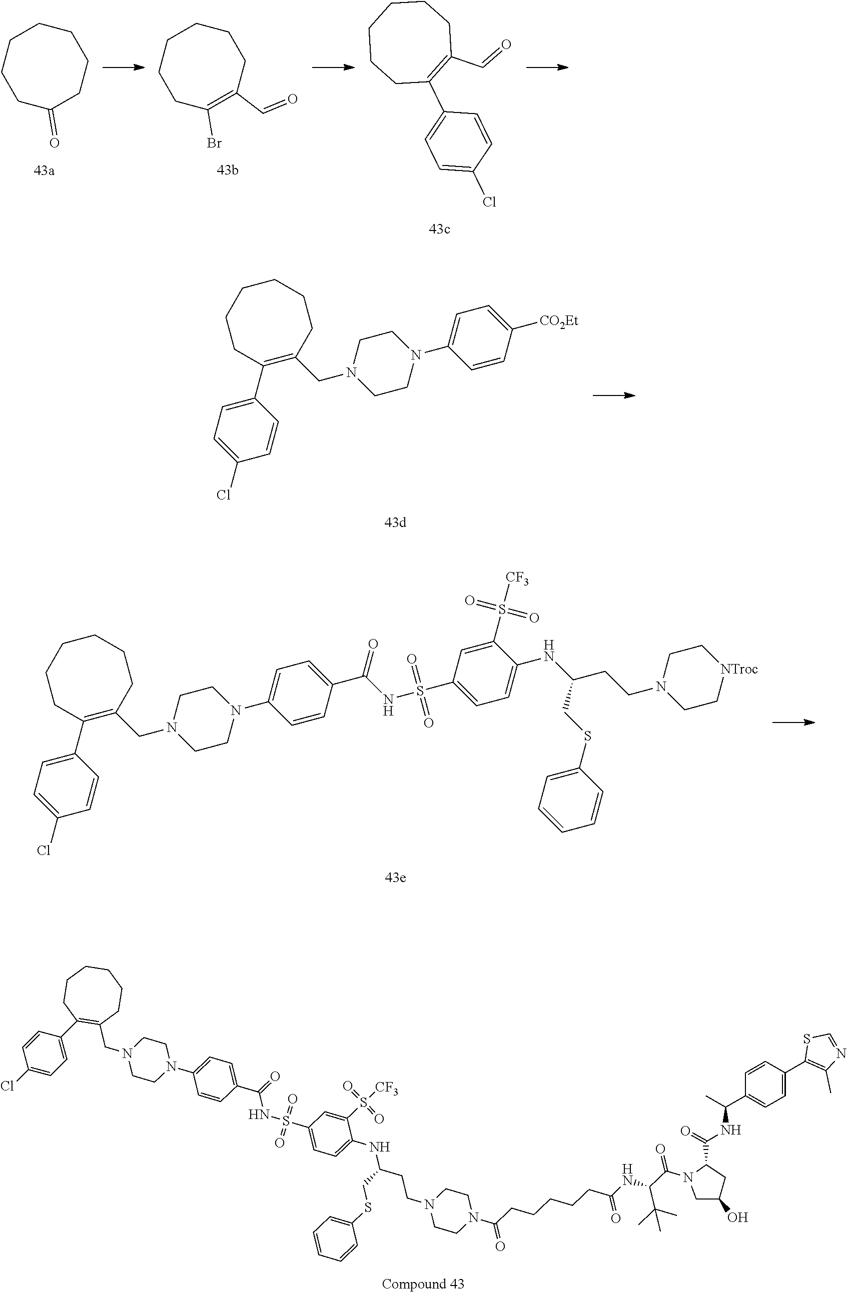

### Step 1: Preparation of 43b

DMF (17.12 g, 234.22 mmol) was added to 80 mL of dichloromethane. Under nitrogen protection, the temperature was controlled to be 0° C.-5° C. PBr₃ (34.54 g, 127.6 mmol) was slowly added dropwise to the above-mentioned reaction solution. The ice-bath was removed and the mixture was slowly warmed to room temperature and stirred for 0.5 h. The reaction system was cooled to 0° C.-5° C. again. A solution of 43a (5.00 g, 39.62 mmol) in dichloromethane (40 mL) was added while the temperature was controlled in the range and then the mixture was warmed to room temperature and reacted for 16 h. The reaction solution was slowly added to 200 mL of ice water, followed by liquid separation. The aqueous phase was extracted with dichloromethane (100 mL×2). The organic phases were combined, washed with 50 mL of saturated aqueous sodium chloride solution, dried over anhydrous sodium sulfate and concentrated under reduced pressure and the crude was separated and purified by chromatographic column on silica gel (pure petroleum ether) to afford 43b (2.20 g, yield: 26%).

### Step 2: Preparation of 43c

43b (2.20 g, 10.13 mmol) and 4-chlorophenylboronic acid (2.38 g, 15.22 mmol) were added to 20 mL of 1,4-dioxane and 2 mL of water. Under nitrogen protection, potassium acetate (2.98 g, 30.36 mmol) and Pd(dppf)Cl₂ (0.25 g, 0.34 mmol) were sequentially added and the mixture was reacted at 90° C. for 1 h. The reaction system was cooled to room temperature and 20 mL of ethyl acetate and 20 mL of water were added, followed by liquid separation. The aqueous phase was extracted with ethyl acetate (25 mL×2). The organic phases were combined, washed with 30 mL of saturated aqueous sodium chloride solution, dried over anhydrous sodium sulfate and concentrated under reduced pressure and the resulting crude was separated and purified by silica gel column chromatography (petroleum ether/ethyl acetate (v/v)=1:0-20:1) to afford 43c (1.60 g, yield: 64%).

LCMS m/z=249.2 [M+1]⁺.

### Step 3: Preparation of 43d

43c (1.60 g, 6.45 mmol), ethyl (4-piperazin-1-yl)benzoate (2.26 g, 9.64 mmol) and acetic acid (2.56 g, 42.67 mmol) were added to 20 mL of tetrahydrofuran. Sodium triacetoxyborohydride (4.09 g, 19.30 mmol) was added and the mixture was reacted at room temperature for 16 h. To the reaction solution was added 100 mL of saturated sodium bicarbonate solution, followed by extraction using ethyl acetate (60 mL×2). The organic phases were combined, washed with 50 mL of saturated aqueous sodium chloride solution, dried over anhydrous sodium sulfate and concentrated under reduced pressure and the resulting crude was separated and purified by silica gel column chromatography (petroleum ether/ethyl acetate (v/v)=1:0-5:1) to afford 43d (2.20 g, yield: 73%).

LCMS m/z=467.6 [M+1]⁺.

### Step 4: Preparation of 43e

43d (2.20 g, 4.71 mmol) was dissolved in mixed solvents of 32 mL of tetrahydrofuran, 16 mL of ethanol and 8 mL of water. Sodium hydroxide (0.75 g, 18.75 mmol) was added and the mixture was reacted at 65° C. for 16 h. The reaction system was cooled to room temperature and concentrated under reduced pressure and 20 mL of water and 20 mL of methyl tert-butyl ether were added. The aqueous phase was separated, adjusted to pH 5 with 1 mol/L hydrochloric acid and extracted with ethyl acetate (15 mL×2). The organic phases were combined, washed with 15 mL of saturated aqueous sodium chloride solution, dried over anhydrous sodium sulfate and concentrated under reduced pressure to afford a crude (0.80 g). Intermediate 2 (0.50 g, 0.68 mmol) was dissolved in 10 mL of dichloromethane. The above-mentioned crude (0.33 g), DMAP (0.17 g, 1.39 mmol) and EDCI (0.26 g, 1.36 mmol) were sequentially added and the mixture was reacted at 35° C. for 12 h. To the reaction system was added 10 mL of saturated aqueous potassium dihydrogen phosphate solution, followed by liquid separation. The aqueous phase was extracted with 10 mL of dichloromethane and the organic phase was dried over anhydrous sodium sulfate and concentrated under reduced pressure. The resulting crude was separated and purified by chromatographic column on silica gel (dichloromethane:methanol (v/v)=50:1-20:1) to afford 43e (0.75 g, yield: 96%).

LCMS m/z=1149.3 [M+1]⁺.

### Step 5: Preparation of Compound 43

43e (0.75 g, 0.65 mmol) was added to 8 mL of tetrahydrofuran and 40 mL of methanol. Zinc powder (3.42 g, 52.62 mmol) and ammonium chloride (1.04 g, 19.44 mmol) were sequentially added and the mixture was reacted at 27° C. for 12 h. The reaction system was filtered over diatomaceous earth. The filtrate was concentrated under reduced pressure and the resulting crude was separated and purified by chromatographic column on silica gel (dichloromethane:methanol (v/v)=10:1) to afford a crude (0.29 g). The above-mentioned crude (0.24 g) was added to 15 mL of DCM. Intermediate 1 (0.16 g, 0.28 mmol), triethylamine (0.076 g, 0.75 mmol) and HATU (0.14 g, 0.37 mmol) were sequentially added and the mixture was reacted at room temperature for 2 h. To the reaction solution was added 10 mL of water, followed by liquid separation. The aqueous phase was extracted with DCM (6 mL×2) and the organic phase was dried over anhydrous sodium sulfate and concentrated under reduced pressure. The resulting crude was separated and purified by chromatographic column on silica gel (dichloromethane:methanol (v/v)=50:1-10:1) to afford a crude, which was then passed through Pre-HPLC (instrument and preparative column: using SHIMADZU LC-20AP preparative liquid phase chromatographic instrument, preparative column model: Phenomenex C18). Mobile phase system: acetonitrile/water (containing 10 mmol/L NH₄HCO₃). Gradient elution method: gradient elution with acetonitrile from 50% to 80% (elution time: 15 min). Lyophilization was performed to afford compound 43 (0.20 g, yield: 46%).

¹H NMR (400 MHz, CDCl₃) δ 8.67 (s, 1H), 8.40-8.30 (m, 1H), 8.13-8.04 (m, 1H), 7.73-7.63 (m, 2H), 7.45-7.21 (m, 12H), 7.12-7.02 (m, 1H), 7.00-6.90 (m, 2H), 6.78-6.66 (m, 2H), 6.66-6.57 (m, 1H), 6.40-6.29 (m, 1H), 5.15-5.00 (m, 1H), 4.80-4.42 (m, 3H), 4.16-4.02 (m, 1H), 3.97-3.82 (m, 1H), 3.75-3.52 (m, 2H), 3.50-3.20 (m, 7H), 3.17-2.80 (m, 4H), 2.60-2.00 (m, 24H), 1.80-1.20 (m, 18H), 1.04 (s, 9H).

LCMS m/z=514.8 [M/3+1]⁺.

Example 44

4-(4-((2-(4-chlorophenyl)cyclohept-1-en-1-yl)methyl)piperazin-1-yl)-N-((4-(((2R)-4-(4-((1-(2-(2,6-dioxopiperidin-3-yl)-1,3-dioxoisoindolin-5-yl)azetidin-3-yl)methyl)piperazin-1-yl)-1-(phenylthio)butan-2-yl)amino)-3-((trifluoromethyl)sulfonyl)phenyl)sulfonyl)benzamide (Compound 44)

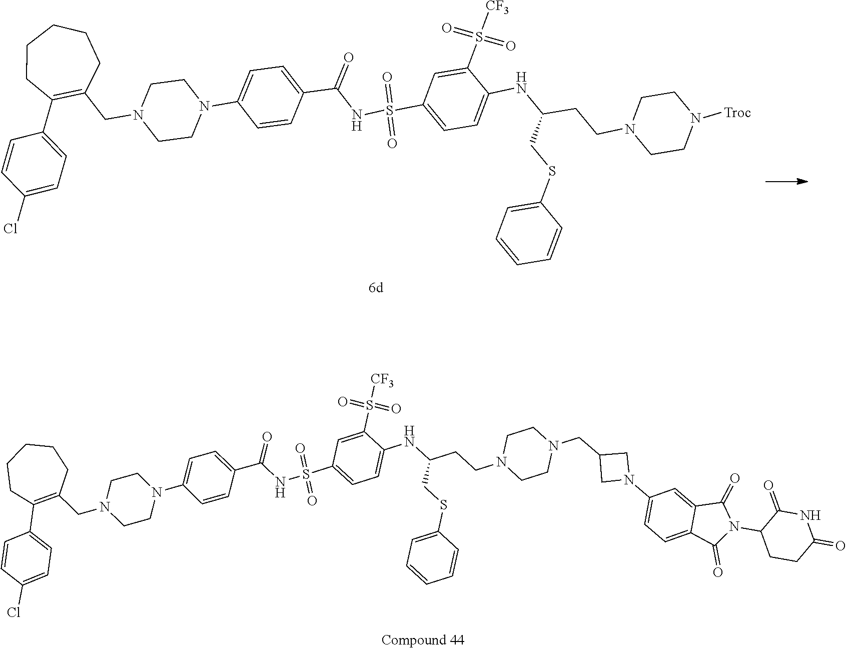

6d (3.00 g, 2.64 mmol) was added to 150 mL of methanol and 30 mL of tetrahydrofuran. Ammonium chloride (4.24 g, 79.27 mmol) and zinc powder (13.81 g, 212.46 mmol) were sequentially added and the mixture was reacted at 27° C. for 19 h. The reaction system was filtered over diatomaceous earth. The filtrate was concentrated under reduced pressure and the resulting crude was separated and purified by chromatographic column on silica gel (dichloromethane:methanol (v/v)=10:1) to afford a crude (2.0 g). The above-mentioned crude (0.20 g) was added to 10 mL of dichloromethane. 1-(2-(2,6-dioxopiperidin-3-yl)-1,3-dioxoisoindolin-5-yl)azetidine-3-carbaldehyde (see CN114292270 for the synthetic method) (0.11 g, 0.32 mmol), acetic acid (0.05 g, 0.83 mmol) and sodium triacetoxyborohydride (0.13 g, 0.61 mmol) were sequentially added and the mixture was reacted at room temperature for 2 h. To the reaction solution was added 10 mL of saturated aqueous sodium bicarbonate solution, followed by liquid separation. The aqueous phase was extracted with DCM (6 mL×2). The organic phases were combined, dried over anhydrous sodium sulfate and concentrated under reduced pressure and the crude was preliminarily separated and purified by chromatographic column on silica gel (dichloromethane:methanol (v/v)=50:1-10:1). The resulting crude was passed through Pre-HPLC (instrument and preparative column: using Glison GX-281 preparative liquid phase chromatographic instrument, preparative column model: Sunfire C18, 5 μm, inner diameter×length=30 mm×150 mm). Preparation method: the crude was dissolved with methanol and dimethyl sulfoxide, and filtered with a 0.45 m filter membrane, to prepare into a sample liquid. Mobile phase system: acetonitrile/water (containing 0.1% TFA). Gradient elution method: gradient elution with acetonitrile from 5% to 60% (elution time: 15 min). The preparative solution was adjusted to pH 7 with saturated aqueous sodium bicarbonate solution and extracted with DCM (20 mL×2). The organic phase was washed with 20 mL of saturated aqueous sodium chloride solution, dried over anhydrous sodium sulfate and concentrated under reduced pressure to afford compound 44 (68 mg, yield: 17%).

¹H NMR (400 MHz, CDCl₃) δ 8.33 (s, 1H), 8.18-8.01 (m, 1H), 7.84-7.45 (m, 3H), 7.40-7.15 (m, 7H), 7.03-6.90 (m, 3H), 6.80-6.55 (m, 4H), 6.50-6.35 (m, 1H), 4.95-4.80 (m, 1H), 4.20-4.00 (m, 2H), 3.98-3.79 (m, 1H), 3.76-3.55 (m, 2H), 3.30-2.90 (m, 6H), 2.85-2.57 (m, 6H), 2.55-2.25 (m, 18H), 2.15-1.95 (m, 3H), 1.90-1.75 (m, 3H), 1.67-1.45 (m, 5H).

LCMS m/z=1284.3 [M+1]⁺.

Example 45

4-(4-((2-(4-chlorophenyl)cyclohept-1-en-1-yl)methyl)piperazin-1-yl)-N-((4-(((2R)-4-(4-((1-(2-(2,6-dioxopiperidin-3-yl)-1,3-dioxoisoindolin-5-yl)piperidin-4-yl)methyl)piperazin-1-yl)-1-(phenylthio)butan-2-yl)amino)-3-((trifluoromethyl)sulfonyl)phenyl)sulfonyl)benzamide (Compound 45)

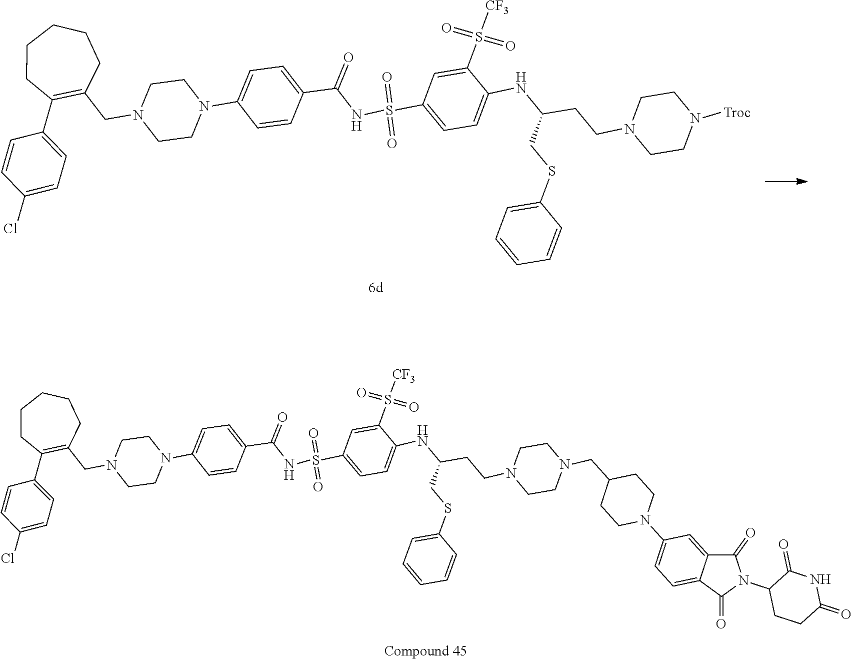

6d (3.00 g, 2.64 mmol) was added to 150 mL of methanol and 30 mL of tetrahydrofuran. Ammonium chloride (4.24 g, 79.27 mmol) and zinc powder (13.81 g, 212.46 mmol) were sequentially added and the mixture was reacted at 27° C. for 19 h. The reaction system was filtered over diatomaceous earth. The filtrate was concentrated under reduced pressure and the resulting crude was separated and purified by chromatographic column on silica gel (dichloromethane:methanol (v/v)=10:1) to afford a crude (2.0 g). The above-mentioned crude (0.20 g) was added to 10 mL of DCM. 1-(2-(2,6-dioxopiperidin-3-yl)-1,3-dioxoisoindolin-5-yl)piperidine-4-carbaldehyde (see CN 114292270 for the synthetic method) (0.12 g, 0.32 mmol), acetic acid (0.050 g, 0.83 mmol) and sodium triacetoxyborohydride (0.13 g, 0.61 mmol) were sequentially added and the mixture was reacted at room temperature for 2 h. To the reaction solution was added 10 mL of saturated aqueous sodium bicarbonate solution, followed by liquid separation. The aqueous phase was extracted with DCM (6 mL×2). The organic phases were combined, dried over anhydrous sodium sulfate and concentrated under reduced pressure and the crude was preliminarily separated and purified by chromatographic column on silica gel (dichloromethane:methanol (v/v)=50:1-10:1). The resulting crude was passed through Pre-HPLC (instrument and preparative column: using Glison GX-281 preparative liquid phase chromatographic instrument, preparative column model: Sunfire C18, 5 μm, inner diameter×length=30 mm×150 mm). Preparation method: the crude was dissolved with methanol and dimethyl sulfoxide, and filtered with a 0.45 m filter membrane, to prepare into a sample liquid. Mobile phase system: acetonitrile/water (containing 0.1% TFA). Gradient elution method: gradient elution with acetonitrile from 5% to 60% (elution time: 15 min). The preparative solution was adjusted to pH 7 with saturated aqueous sodium bicarbonate solution and extracted with DCM (20 mL×2). The organic phase was washed with 20 mL of saturated aqueous sodium chloride solution, dried over anhydrous sodium sulfate and concentrated under reduced pressure to afford compound 45 (0.13 g, yield: 31%).

¹H NMR (400 MHz, CDCl₃) δ 8.31 (s, 1H), 8.16-7.98 (m, 1H), 7.82-7.55 (m, 3H), 7.40-7.15 (m, 8H), 7.05-6.83 (m, 4H), 6.80-6.50 (m, 3H), 4.98-4.84 (m, 1H), 4.00-3.75 (m, 3H), 3.30-2.60 (m, 13H), 2.60-2.15 (m, 20H), 2.15-1.95 (m, 3H), 1.92-1.75 (m, 4H), 1.68-1.46 (m, 7H).

LCMS m/z=657.2 [M/2+1]⁺.

Example 46: Preparation of Compound 46

4-(4-((2-(4-chlorophenyl)cyclohept-1-en-1-yl)methyl)piperazin-1-yl)-N-((4-(((2R)-4-(4-((1-(4-(2,6-dioxopiperidin-3-yl)phenyl)azetidin-3-yl)methyl)piperazin-1-yl)-1-(phenylthio)butan-2-yl)amino)-3-((trifluoromethyl)sulfonyl)phenyl)sulfonyl)benzamide (Compound 46)

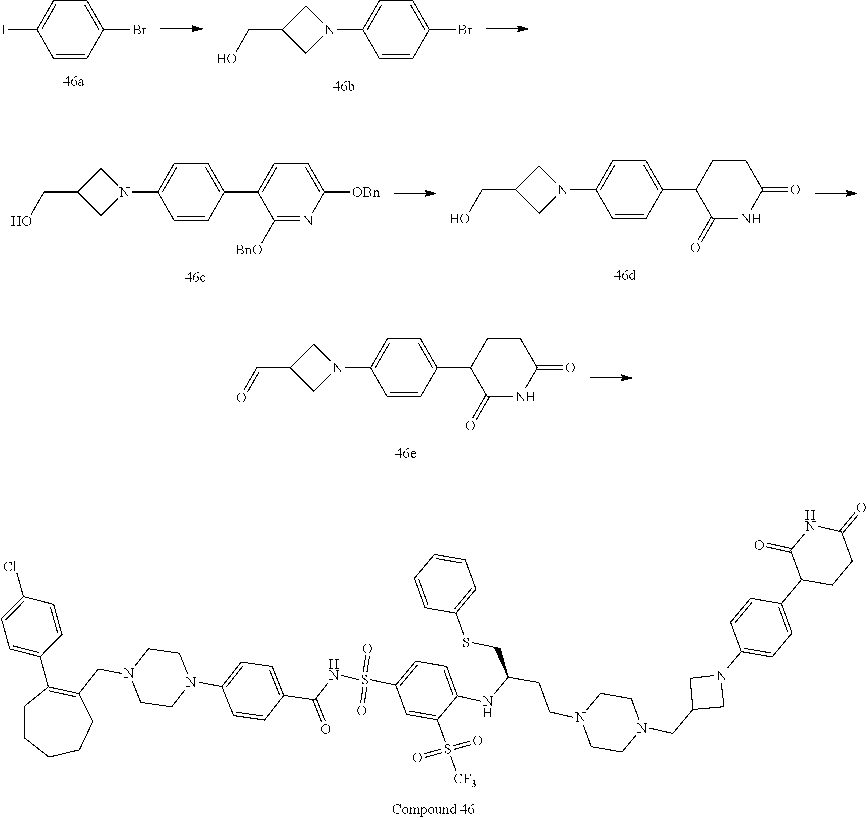 [SMILES: Brc1ccc(I)cc1 ; OCC1CN(c2ccc(Br)cc2)C1 ; OCC1CN(c2ccc(-c3ccc(OCc4ccccc4)nc3OCc3ccccc3)cc2)C1 ; O=C1CCC(c2ccc(N3CC(CO)C3)cc2)C(=O)N1 ; O=CC1CN(c2ccc(C3CCC(=O)NC3=O)cc2)C1 ; O=C1CCC(c2ccc(N3CC(CN4CCN(CC[C@H](CSc5ccccc5)Nc5ccc(S(=O)(=O)NC(=O)c6ccc(N7CCN(CC8=C(c9ccc(Cl)cc9)CCCCC8)CC7)cc6)cc5S(=O)(=O)C(F)(F)F)CC4)C3)cc2)C(=O)N1]

### Step 1: Preparation of 46b

46a (15.00 g, 53.02 mmol) was added to 150 mL of DMSO and under nitrogen protection, (azetidin-3-yl)methanol hydrochloride (7.86 g, 63.60 mmol), L-proline (2.44 g, 21.19 mmol), potassium carbonate (21.98 g, 159.04 mmol) and CuI (2.02 g, 10.61 mmol) were added and the system was subjected to nitrogen replacement three times and reacted at 100° C. for 16 h. The reaction system was cooled to room temperature and 100 mL of water was added. The mixture was extracted with ethyl acetate (60 mL×3). The organic phase was washed with 50 mL of saturated aqueous sodium chloride solution, dried over anhydrous sodium sulfate and concentrated under reduced pressure and the resulting crude was separated and purified by chromatographic column on silica gel (petroleum ether:ethyl acetate (v/v)=20:1-5:1) to afford 46b (5.60 g, yield: 44%).

LCMS m/z=242.1 [M+1]⁺.

### Step 2: Preparation of 46c

46b (5.10 g, 21.15 mmol) was added to 60 mL of 1,4-dioxane and 15 mL of water. Under nitrogen protection, 2,6-bis(benzyloxy)-3-(4,4,5,5-tetramethyl-1,3,2-dioxaborolan-2-yl)pyridine (26.37 g, 63.19 mmol) (see WO 2021262812 for the synthetic method), cesium carbonate (20.59 g, 63.19 mmol) and Pd(dppf)Cl₂ (0.77 g, 1.05 mmol) were added and the system was subjected to nitrogen replacement three times and reacted at 100° C. for 16 h. The reaction system was cooled to room temperature, filtered over diatomaceous earth, and washed with 50 mL of ethyl acetate. To the filtrate was added 50 mL of water and the mixture was extracted with ethyl acetate (50 mL×3). The organic phase was washed with 50 mL of saturated aqueous sodium chloride solution, dried over anhydrous sodium sulfate and concentrated under reduced pressure and the resulting crude was separated and purified by chromatographic column on silica gel (petroleum ether:ethyl acetate (v/v)=20:1-5:1) to afford 46c (9.10 g, yield: 95%).

LCMS m/z=453.3 [M+1]⁺.

### Step 3: Preparation of 46d

46c (9.10 g, 20.12 mmol) was added to 100 mL of methanol. 10% Pd/C (5.00 g) was added and the system was subjected to nitrogen replacement three times and reacted at room temperature under the atmosphere of hydrogen balloon for 16 h. The reaction system was filtered over diatomaceous earth and then washed with mixed solvents of dichloromethane and methanol (v/v)=10:1. The filtrate was concentrated under reduced pressure and to the resulting crude were added 40 mL of ethyl acetate and 40 mL of petroleum ether. The mixture was stirred at room temperature for 0.5 h and then filtered. The filter cake was dried under reduced pressure to afford crude 46d (3.10 g).

LCMS m/z=275.2 [M+1]⁺.

### Step 4: Preparation of 46e

The above-mentioned crude 46d (0.20 g) was added to 5 mL of DMSO. 2-iodoxybenzoic acid (0.33 g, 1.18 mmol) was added and the mixture was reacted at 50° C. for 1 h. The reaction system was cooled to room temperature and 10 mL of ethyl acetate and 10 mL of water were added, followed by liquid separation. The aqueous phase was extracted with ethyl acetate (5 mL×2). The organic phases were combined, washed with 10 mL of saturated aqueous sodium chloride solution, dried over anhydrous sodium sulfate and concentrated under reduced pressure to afford crude 46e (0.15 g).

### Step 5: Preparation of Compound 46

6d (3.00 g, 2.64 mmol) was added to 150 mL of methanol and 30 mL of tetrahydrofuran. Ammonium chloride (4.24 g, 79.27 mmol) and zinc powder (13.81 g, 212.46 mmol) were sequentially added and the mixture was reacted at 27° C. for 19 h. The reaction system was filtered over diatomaceous earth. The filtrate was concentrated under reduced pressure and the resulting crude was separated and purified by chromatographic column on silica gel (dichloromethane:methanol (v/v)=10:1) to afford a crude (2.0 g). The above-mentioned crude (0.15 g) was added to 10 mL of dichloromethane. Crude 46e (0.15 g), acetic acid (0.038 g, 0.63 mmol) and sodium triacetoxyborohydride (0.10 g, 0.472 mmol) were sequentially added and the mixture was reacted at room temperature for 2 h. To the reaction solution was added 10 mL of saturated aqueous sodium bicarbonate solution, followed by liquid separation. The aqueous phase was extracted with dichloromethane (6 mL×2). The organic phases were combined, dried over anhydrous sodium sulfate and concentrated under reduced pressure and the resulting crude was separated and purified by chromatographic column on silica gel (dichloromethane:methanol (v/v)=50:1-10:1) to afford a crude, which was then separated and purified by preparative liquid phase chromatography to afford compound 46 (0.13 g, yield over three steps from compound 46c: 8%).

¹H NMR (400 MHz, CDCl₃) δ 8.40-8.29 (m, 1H), 8.10 (dd, 1H), 7.93 (s, 1H), 7.69-7.60 (m, 2H), 7.41-7.20 (m, 7H), 7.10-6.92 (m, 5H), 6.84-6.72 (m, 2H), 6.63 (d, 1H), 6.46-6.37 (m, 2H), 4.05-3.83 (m, 3H), 3.74-3.65 (m, 1H), 3.57-3.47 (m, 2H), 3.32-3.22 (m, 4H), 3.14-2.89 (m, 3H), 2.84 (s, 2H), 2.77-2.03 (m, 25H), 1.88-1.47 (m, 7H).

LCMS m/z=608.3 [M/2+1]⁺.

Example 47: Preparation of Compound 47

4-(4-((2-(4-chlorophenyl)cyclohept-1-en-1-yl)methyl)piperazin-1-yl)-N-((4-(((2R)-4-(4-(2-(2,6-dioxopiperidin-3-yl)-1,3-dioxoisoindolin-5-yl)piperazin-1-yl)-1-(phenylthio)butan-2-yl)amino)-3-((trifluoromethyl)sulfonyl)phenyl)sulfonyl)benzamide (Compound 47)

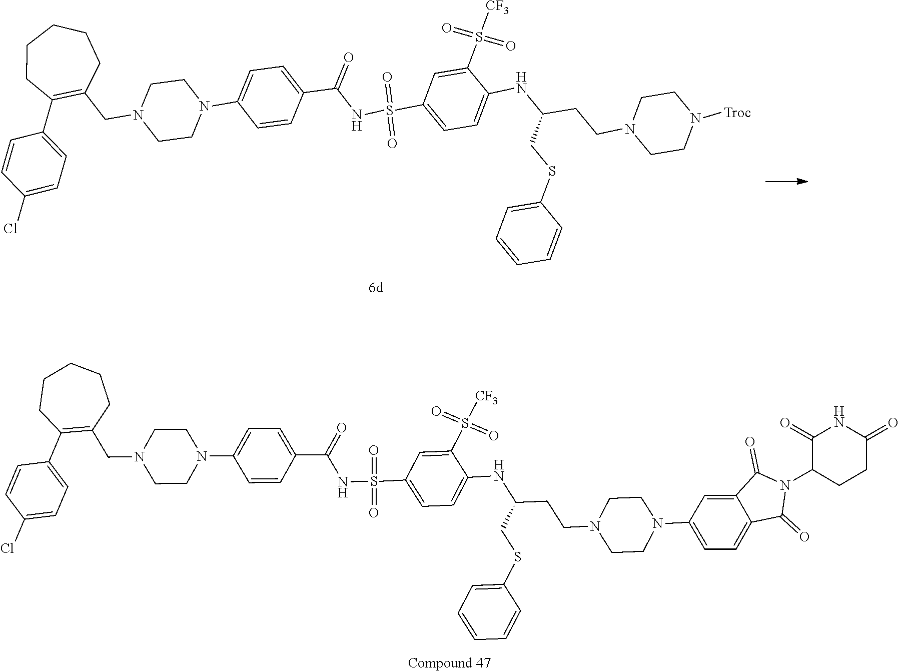

6d (3.00 g, 2.64 mmol) was added to 150 mL of methanol and 30 mL of tetrahydrofuran. Ammonium chloride (4.24 g, 79.27 mmol) and zinc powder (13.81 g, 212.46 mmol) were sequentially added and the mixture was reacted at 27° C. for 19 h. The reaction system was filtered over diatomaceous earth. The filtrate was concentrated under reduced pressure and the resulting crude was separated and purified by chromatographic column on silica gel (dichloromethane:methanol (v/v)=10:1) to afford a crude (2.0 g). The above-mentioned crude (0.10 g) was added to 5 mL of dimethyl sulfoxide. 2-(2,6-dioxopiperidin-3-yl)-5-fluoroisoindoline-1,3-dione (0.083 g, 0.30 mmol) and N,N-diisopropylethylamine (0.065 g, 0.50 mmol) were sequentially added and the mixture was reacted at 100° C. for 16 h. The reaction system was cooled to room temperature and 10 mL of water and 10 mL of ethyl acetate were added, followed by liquid separation. The aqueous phase was extracted with ethyl acetate (6 mL×2). The organic phases were combined, dried over anhydrous sodium sulfate and concentrated under reduced pressure and the resulting crude was separated and purified by chromatographic column on silica gel (dichloromethane:methanol (v/v)=50:1-10:1) to afford a crude, which was then separated and purified by preparative liquid phase chromatography to afford compound 47 (20 mg, yield: 5%).

¹H NMR (400 MHz, CDCl₃) δ 8.40-8.35 (m, 1H), 8.13 (dd, 1H), 8.03-7.92 (m, 1H), 7.69 (d, 1H), 7.65-7.57 (m, 2H), 7.44-7.20 (m, 8H), 7.17-7.09 (m, 1H), 7.06-6.94 (m, 3H), 6.83-6.74 (m, 2H), 6.62 (d, 1H), 4.99-4.88 (m, 1H), 4.00-3.87 (m, 1H), 3.49-3.20 (m, 8H), 3.18-2.97 (m, 2H), 2.96-2.65 (m, 5H), 2.65-2.07 (m, 16H), 1.89-1.40 (m, 7H).

LCMS m/z=608.8 [M/2+1]⁺.

Example 48: Preparation of Compound 48

(3R,5S)-1-((S)-2-(7-(4-((R)-3-((4-(N-(4-(4-((2-(4-chlorophenyl)cyclohept-1-en-1-yl)methyl)piperazin-1-yl)benzoyl)sulfamoyl)-2-((trifluoromethyl)sulfonyl)phenyl)amino)-4-(phenylthio)butyl)piperazin-1-yl)-7-oxoheptanamido)-3,3-dimethylbutanoyl)-5-(((S)-1-(4-(4-methylthiazol-5-yl)phenyl)ethyl)carbamoyl) pyrrolidin-3-yl acetate (Compound 48)

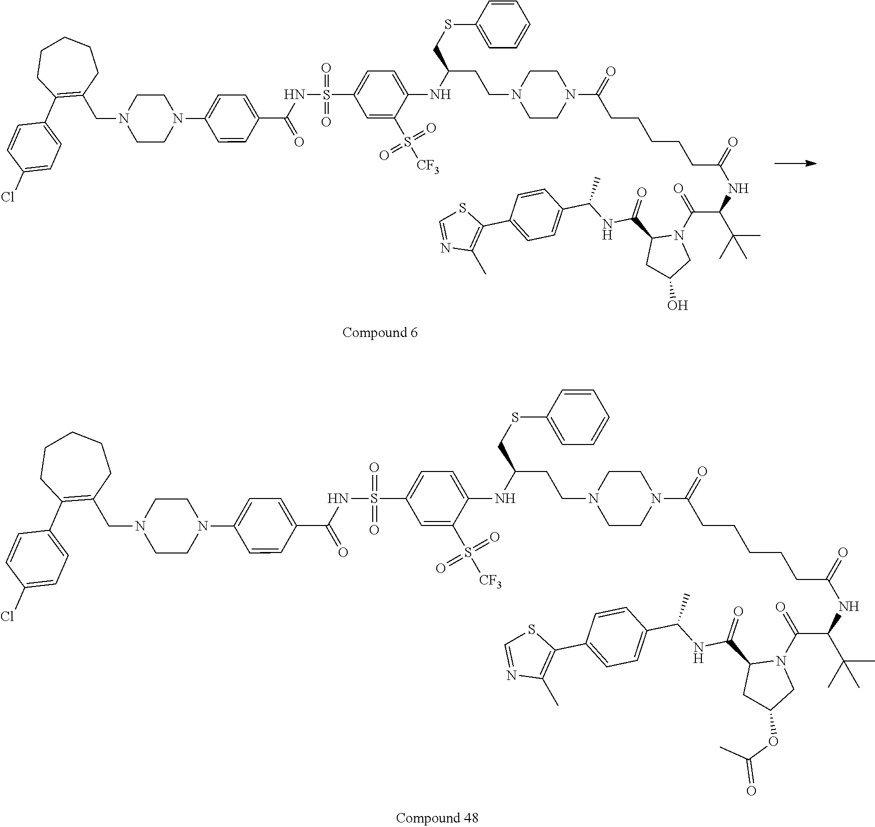 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCCCC6)CC5)cc4)cc3S(=O)(=O)C(F)(F)F)CC2)C(C)(C)C)cc1 ; CC(=O)O[C@@H]1C[C@@H](C(=O)N[C@@H](C)c2ccc(-c3scnc3C)cc2)N(C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCCCC6)CC5)cc4)cc3S(=O)(=O)C(F)(F)F)CC2)C(C)(C)C)C1]

Free-form compound 6 (50 mg, 0.033 mmol) was dissolved in 3 mL of dichloromethane. 4-dimethylaminopyridine (8.1 mg, 0.066 mmol) and acetic anhydride (33 mg, 0.32 mmol) were added and the mixture was reacted at room temperature for 2 h. The reaction solution was concentrated under reduced pressure and the crude product was subjected to Pre-HPLC (instrument and preparative column: using SHIMADZU LC-20AP preparative liquid phase chromatographic instrument, preparative column model: Phenomenex C18). Preparation method: the crude was dissolved with methanol and dimethyl sulfoxide, and filtered with a 0.45 m filter membrane, to prepare into a sample liquid. Mobile phase system: acetonitrile/water (containing 0.05% NH₄HCO₃). Gradient elution method: gradient elution with acetonitrile from 60% to 90% (elution time: 15 min). Lyophilization was performed to afford compound 48 (30 mg, yield: 58%).

¹H NMR (400 MHz, CDCl₃) δ 8.68 (s, 1H), 8.40-8.32 (m, 1H), 8.16-8.06 (s, 1H), 7.69-7.58 (m, 2H), 7.46-7.20 (m, 12H), 7.17-7.08 (m, 1H), 7.02-6.93 (m, 2H), 6.84-6.71 (m, 2H), 6.64-6.54 (m, 1H), 6.18-6.08 (m, 1H), 5.43-5.27 (m, 1H), 5.15-4.97 (m, 1H), 4.77-4.67 (m, 1H), 4.62-4.54 (m, 1H), 4.13-4.00 (m, 1H), 3.97-3.56 (m, 3H), 3.54-3.17 (m, 7H), 3.16-2.95 (m, 2H), 2.94-2.65 (m, 3H), 2.55-1.97 (m, 26H), 1.90-1.30 (m, 16H), 1.02 (s, 9H).

LCMS m/z=785.5 [M/2+1]⁺.

Experimental Examples

### 1. BCL-xL and BCL-2 Degradation Activity Studies in MOLT-4 Cells

MOLT-4 cells are acute lymphoblastic leukemia cell lines in humans. The cells were purchased from ATCC. Culture conditions: RPMI-1640+10% FBS+1% penicillin-streptomycin solution. The cells were cultured in a 37° C., 5% CO₂ incubator. The cells were plated in a 12-well plate, with 2×10⁵ cells/well. After plating, compounds at different concentrations were added and cultured in a 37° C., 5% CO₂ incubator for 16 h. After the completion of culture, the cells were harvested and RIPA lysis buffer (beyotime, Cat. P0013B) was added. The cells were lysed on ice for 15 minutes and centrifuged at 12000 rpm at 4° C. for 10 minutes. The supernatant protein samples were collected and the protein was quantified by using the BCA kit (Beyotime, Cat. P0009). Then the protein was diluted to 0.8 mg/mL. The expressions of BCL-xL (CST, Cat. 2764S), BCL-2 (CST, Cat. 15071S) and the internal reference β-actin (CST, Cat. 3700S) were detected using a fully automated western blot quantitative analyzer (Proteinsimple). The expression level of BCL-xL or BCL-2 relative to the internal reference was calculated by using Compass software, and the DC₅₀ value was calculated by using GraphPad Prism 8.0 software according to formula (1), wherein the Protein_{administration} denoted the relative expression level of BCL-xL or BCL-2 in administration groups at different doses, and the Protein_{vehicle} denoted the relative expression level of BCL-xL or BCL-2 in the vehicle control group.

Protein %=Protein_{administration}/Protein_{vehicle}×100% formula (1)

The results of DC₅₀ values for the degradation of BCL-xL proteins in MOLT-4 cells are shown in Table 1.

TABLE 1
DC₅₀ values of the compounds according to the present
invention for the degradation of BCL-xL proteins

| Serial No. | Compound No. | DC₅₀ (nM) |
|---|---|---|
| 1 | Control compound | >20 |
| 2 | Compound 6 | 8.4 |
| 3 | Compound 8 | 8.8 |
| 5 | Compound 10 | 3.7 |
| 6 | Compound 11 | 5.3 |
| 9 | Compound 14 | 8.1 |
| 10 | Compound 15 | 3.6 |
| 12 | Compound 19 | 10.7 |

Conclusion: the compounds of the present invention had a given degradation effect on the BCL-xL proteins in MOLT-4 cells.

### Structure of the Control Compound (DT2216):

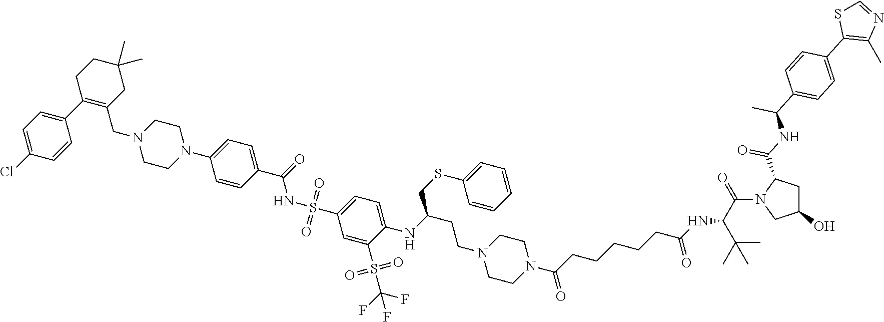 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)(C)C)cc1]

### 2. Cell Proliferation Activity Studies in MOLT-4 Cells

MOLT-4 cells are acute lymphoblastic leukemia cell lines in humans. The cells were purchased from ATCC. Culture conditions: RPMI-1640+10% FBS+1% penicillin-streptomycin solution. The cells were cultured in a 37° C., 5% CO₂ incubator. The cells were plated in a 96-well plate, with 5×10³ cells/well. After plating, compounds at different concentrations were added and cultured in a 37° C., 5% CO₂ incubator for 72 h. After the completion of culture, a reagent (Promega, G7573) for detecting the cell viability was added. The mixture was uniformly mixed for 2 minutes and then incubated at room temperature for 10 minutes. The luminescence signal was detected with a multimode plate reader (BMG, PHERAstar FSX). The luminescence readings were processed using GraphPad Prism 8.0 software and the IC₅₀ values of the compounds for the inhibition of cell proliferation and the maximum inhibition rate were calculated according to formula (2) and formula (3), respectively, wherein T_{administration} denotes the cell signal readings obtained after incubation with the compounds for 72 h, and T_{vehicle} denotes the cell signal readings obtained after incubation with the vehicle control for 72 h.

$$Growth \% = {T}_{administration} / {T}_{vehicle} \times 100 \% \qquad formula ( 2 )$$

Calculation of Max inhibition %: after the processing according to formula (2), the inhibition rate of the compounds at the highest concentration was calculated.

$$Max inhi . \% = 1 - Growth \% \qquad formula ( 3 )$$

The results of IC₅₀ values for the inhibition of MOLT-4 cell proliferation are shown in Table 2.

TABLE 2
IC50 values of the compounds according to the present
invention for the inhibition of MOLT-4 cell proliferation

| Serial | | |
|---|---|---|
| No. | Compound No. | IC₅₀ (nM) |
| 1 | Compound 2 | A |
| 2 | Trifluoroacetate of compound 4 | A |
| 3 | Compound 5 | A |
| 4 | Trifluoroacetate of compound 6 | A |
| 5 | Compound 7 | A |
| 6 | Compound 8 | A |
| 7 | Trifluoroacetate of compound 9 | A |
| 8 | Compound 10 | A |
| 9 | Compound 11 | A |
| 10 | Compound 12 | A |
| 11 | Compound 13 | A |
| 12 | Compound 14 | A |
| 13 | Compound 15 | A |
| 14 | Compound 16 | A |
| 15 | Compound 17 | A |
| 16 | Compound 18 | A |
| 17 | Compound 19 | A |
| 18 | Compound 20 | A |
| 19 | Compound 21 | A |
| 20 | Compound 22 | A |
| 21 | Compound 23 | A |
| 22 | Compound 24 | A |
| 23 | Compound 25 | A |
| 24 | Compound 26 | A |
| 25 | Compound 27 | A |
| 26 | Compound 28 | A |
| 27 | Compound 29 | A |
| 28 | Compound 30 | A |
| 29 | Compound 31 | A |
| 30 | Compound 32 | A |
| 31 | Compound 33 | A |
| 32 | Compound 34 | A |
| 33 | Compound 35 | A |
| 34 | Compound 36 | A |
| 35 | Compound 37 | A |
| 36 | Compound 38 | A |
| 37 | Compound 39 | A |
| 38 | Compound 40 | A |
| 39 | Compound 41 | A |
| 40 | Compound 42 | A |
| 41 | Compound 43 | A |
| 42 | Compound 44 | A |
| 43 | Compound 45 | A |

A < 500 nM; 500 nM ≤ B ≤ 5000 nM; C > 5000 nM.
Conclusion: the compounds according to the present invention had a given inhibitory effect on the proliferation of MOLT-4 cells.

### 3. Growth Inhibition Experiment in MOLT-4 Xenograft Tumor Models in Nude Mice

### Cell Inoculation and Animal Administration:

MOLT-4 cells are acute lymphoblastic leukemia cell lines in humans. The cells were purchased from ATCC and cultured in RPMI-1640+10% FBS+1% o penicillin-streptomycin solution in a 37° C., 5% CO₂ incubator. When the cells were in the exponential growth phase, the cells were harvested and counted. An equal volume of matrigel was added. Female SCID Beige mice (4-6 weeks old, 14-16 g, Beijing Vital River Laboratory Animal Technology Co., Ltd.) were inoculated subcutaneously in the right flank with the cells (200 μL, containing 1×10⁷ MOLT-4 cells and 50% matrigel). When the tumor volume reached 150-200 mm³, the mice were randomly grouped for administration according to the tumor volume and body weight of the mice. Three administration dosages were provided: 5 mpk, 15 mpk and 50 mpk. The compounds were injected intraperitoneally (ip) weekly (qw). The mice in all the experimental groups (10 mice in each group) were continuously administered for 4 weeks.

### Experiment Observation and Endpoint:

After inoculation, the body weights of the mice were measured three times a week and the long (a) and short (b) diameters of the tumors were measured. The tumor volume was calculated according to the formula: V=ab²/2. At day 28 after the administration, the experiment was terminated.

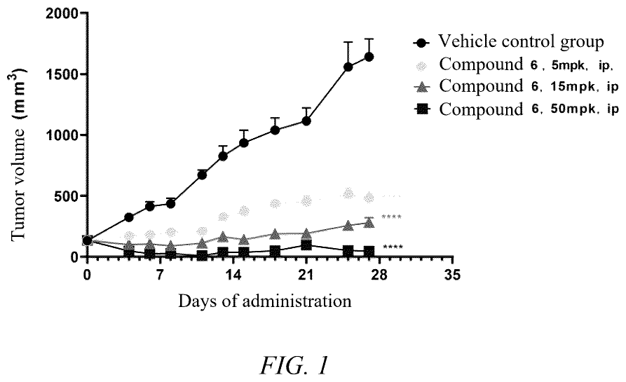

Experimental results: in MOLT-4 xenograft tumor models, compound 6 had significant tumor-suppressing efficacy on MOLT-4 xenograft tumors in mice relative to the vehicle control group, and with the increase of the administration dosage, the tumor-suppressing effects grew stronger. See FIG. 1.

### 4. Pharmacokinetic Test in Rats

Experimental objective: in this test, a single dose of test compounds was administered to SD rats intragastrically, and the concentrations of the test compounds in plasma of the rats were measured to evaluate the pharmacokinetic characteristics of the test compounds in the rats.

Experimental animals: male SD rats, 200-250 g, 6-8 weeks old, 3 rats/compound, purchased from Chengdu Ddossy Experimental Animals Co., Ltd.

Experimental method: on the day of the test, 3 SD rats were randomly grouped according to their body weight. The animals were fasted with water available for 12 to 14 h one day before the administration of a test compound, and were fed 4 hours after the administration.

TABLE 3

| | | Administration information | | | | | | |
|---|---|---|---|---|---|---|---|---|
| | | | Administration | Administration | Administration | | | |
| | Quantity | Test | dosage* | concentration | volume | Collected | Mode of | |
| Group | Male | compound | (mg/kg) | (mg/mL) | (mL/kg) | samples | administration | Vehicle |
| G1 | 3 | Compound | 5 | 0.5 | 10 | Plasma | Oral | 5% DMSO + |
| | | according | | | | | (intragastrically) | 5% Solutol + |
| | | to the | | | | | | 30% PEG400 + |
| | | present | | | | | | 60% (20% |
| | | invention | | | | | | SBE-CD) |

*Dosage is calculated based on free base.

Sampling: before and after the administration, 0.1 mL of blood samples were drawn from the orbits of the animals under isoflurane anesthesia, and placed in an EDTAK2 centrifuge tube. Centrifugation was carried out at 5000 rpm at 4° C. for 10 min, and the plasma was collected.

Time points for plasma collection in PO group: 0, 5 min, 15 min, 30 min, 1 h, 2 h, 4 h, 6 h, 8 h, and 24 h.

Before analysis and detection, all samples were stored at −60° C. The samples were analyzed quantitatively by LC-MS/MS.

Conclusion: the compound of the present invention had good oral absorption in rats.

### 5. Pharmacokinetic Test in Mice

Experimental objective: in this test, a single dose of test compounds was administered to C57 mice intragastrically, and the concentrations of the test compounds in plasma of the mice were measured to evaluate the pharmacokinetic characteristics of the test compounds in the mice.

Experimental animals: male C57 mice, 20-25 g, 6-8 weeks old, 3 mice/compound. purchased from Chengdu Ddossy Experimental Animals Co., Ltd.

Experimental method: on the day of the test, 3 C57 mice were randomly grouped according to their body weight. The animals were fasted with water available for 12 to 14 h one day before the administration of a test compound, and were fed 4 hours after the administration.

TABLE 4

| | | Administration information | | | | | | |
|---|---|---|---|---|---|---|---|---|
| | | | Administration | Administration | Administration | | | |
| | Quantity | Test | dosage* | concentration | volume | Collected | Mode of | |
| Group | Male | compound | (mg/kg) | (mg/mL) | (mL/kg) | sample | administration | Vehicle |
| G1 | 3 | Compound | 5 | 0.5 | 10 | Plasma | Oral | 5% DMSO + |
| | | according | | | | | (intragastrically) | 5% Solutol + |
| | | to the | | | | | | 30% PEG400 + |
| | | present | | | | | | 60% (20% |
| | | invention | | | | | | SBE-β-CD) |

*Dosage is calculated based on free base.

Sampling: before and after the administration, 0.03 mL of blood samples were drawn from the orbits of the animals under isoflurane anesthesia, and placed in an EDTAK2 centrifuge tube. Centrifugation was carried out at 5000 rpm at 4° C. for 10 min, and the plasma was collected.

Time points for plasma collection in PO group: 0, 5 min, 15 min, 30 min, 1 h, 2 h, 4 h, 7 h, and 24 h.

Before analysis and detection, all samples were stored at −60° C. The samples were analyzed quantitatively by LC-MS/MS.

Conclusion: the compound of the present invention had good oral absorption in mice.

### 6. Pharmacokinetic Test in Monkeys

Experimental animals: male cynomolgus monkeys, 3-5 kg, 3-6 years old, 2 monkeys/compound. Purchased from Suzhou Xishan Biotechnology Inc.

Experimental method: on the day of the test, 2 monkeys were randomly grouped according to their body weights. The animals were fasted with water available for 14 to 18 h one day before the administration of a test compound, and were fed 4 hours after the administration.

TABLE 5
Administration information

| | | Administration information | | | | | |
|---|---|---|---|---|---|---|---|
| | | | Administration | Administration | Administration | | |
| | Quantity | Test | dosage | concentration | volume | Collected | Mode of |
| Group | Male | compound | (mg/kg) | (mg/mL) | (mL/kg) | samples | administration |
| G1 | 2 | Compound | 2 | 2 | 1 | Plasma | Intravenously |
| | | according | | | | | |
| | | to the | | | | | |
| | | present | | | | | |
| | | invention | | | | | |

Notes:
vehicle for intravenous administration: 5% DMSO + 95% (3% Tween 80 in PBS);
*Dosage is calculated based on free base.

Before and after the administration, 1.0 mL of blood samples were drawn from the limb veins and placed in an EDTAK2 centrifuge tube. Centrifugation was carried out at 5000 rpm at 4° C. for 10 min, and the plasma was collected. Blood collection time points for the intravenous group were: 0, 5 min, 15 min, 30 min, 1 h, 2 h, 4 h, 6 h, 8 h, 10 h, 12 h, 24 h, and 48 h. Before analysis and detection, all samples were stored at −80° C., and a quantitative analysis of samples was performed using LC-MS/MS.

TABLE 6
Pharmacokinetic parameters of the compound according
to the present invention in plasma of monkeys

| Test | Mode of | AUC₀₋ₜ | |
|---|---|---|---|
| compound | administration | (hr*ng/mL) | T_{1/2} (h) |
| Compound 6 | i.v. (2 mg/kg) | 54628 ± 5909 | 9.83 ± 2.2 |

Conclusion: the compound according to the present invention exhibited good pharmacokinetic characteristics in monkeys after intravenous administration.

### 7. Liver Microsomal Stability Test

In this experiment, liver microsomes of five species, including human, monkey, dog, rat, and mouse, were used as in vitro models to evaluate the metabolic stability of the test compound.

At 37° C., 1 μM of the test compound was co-incubated with microsomal protein and coenzyme NADPH. At given time points of the reaction (5 min, 10 min, 20 min, 30 min, and 60 min), the reaction was terminated by adding ice-cold acetonitrile containing an internal standard. The LC-MS/MS method was used to measure the concentration of the test compound in the sample. Ti/2 was calculated using the natural logarithm (ln) of the residual rate of the drug in the incubation system and the incubation time. In addition, the intrinsic clearance in liver microsomes CL_{int(mic)} and the intrinsic clearance in liver CL_{int(Liver)} were further calculated.

Conclusion: the compound according to the present invention had good stability in liver microsomes.

### 8. CYP450 Enzyme Inhibition Test

The purpose of this study was to evaluate the effect of the test compound on the activity of five isoenzymes (CYP1A2, CYP2C9, CYP2C19, CYP2D6 and CYP3A4) of human liver microsomal cytochrome P450 (CYP) by using an in vitro testing system. The specific probe substrates of CYP450 isoenzymes were incubated with human liver microsomes and test compounds of different concentrations, and reduced nicotinamide adenine dinucleotide phosphate (NADPH) was added to initiate the reaction. After the completion of the reaction, the sample was treated and liquid chromatography-tandem mass spectrometry (LC-MS/MS) was used to quantitatively detect metabolites produced by specific substrates, changes in CYP enzyme activity were determined, and IC₅₀ value was calculated to evaluate the inhibitory potential of the test compound on each CYP enzyme subtype.

Conclusion: the compound according to the present invention did not exhibit significant inhibitory effects on the five isoforms of human liver microsomal cytochrome P450.

9. Test for hERG Potassium Ion Channel

Experimental platform: electrophysiological manual patch-clamp system

Cell line: Chinese hamster ovary (CHO) cell lines stably expressing hERG potassium ion channel

Experimental method: in CHO (Chinese Hamster Ovary) cells stably expressing hERG potassium channel, whole cell patch-clamp technique was used to record hERG potassium channel current at room temperature. The glass microelectrode was made of a glass electrode blank (BF150-86-10, Sutter) by a puller. The tip resistance after filling the liquid in the electrode was about 2-5 MΩ. The glass microelectrode can be connected to the patch-clamp amplifier by inserting the glass microelectrode into an amplifier probe. The clamping voltage and data recording were controlled and recorded by the pClamp 10 software through a computer. The sampling frequency was 10 kHz, and the filtering frequency was 2 kHz. After the whole cell records were obtained, the cells were clamped at −80 mV, and the step voltage that induced the hERG potassium current (I_{hERG}) was depolarized from −80 mV to +20 mV for 2 s, then repolarized to −50 mV, and returned to −80 mV after 1 s. This voltage stimulation was given every 10 s, and the administration process was started after the hERG potassium current was confirmed to be stable (at least 1 minute). The compound was administered for at least 1 minute at each test concentration, and at least 2 cells (n≥2) were tested at each concentration.

Data processing: data analysis processing was carried out by using pClamp 10, GraphPad Prism 5 and Excel software. The inhibition degree of hERG potassium current (peak value of hERG tail current induced at −50 mV) at different compound concentrations was calculated by the following formula:

$$Inhibition \% = [ 1 - ( I / Io ) ] \times 100 \%$$

wherein, Inhibition % represents the percentage of inhibition of hERG potassium current by the compound, and I and Io represent the amplitude of hERG potassium current after and before the administration, respectively.

Conclusion: the compound according to the present invention did not exhibit significant inhibitory activity on the hERG potassium current.

10. Effect on Platelet in Mice after Single Intravenous Injection

Experimental animal: 78 SD rats, with an equal number of males and females, male (♂): 290-380 g, female (♀): 220-290 g, 8-10 weeks old, purchased from Zhejiang Vital River Laboratory Animal Technology Co., Ltd.

Experimental protocol: a clear solution was formulated using a vehicle (500 DMS+95% (3O % Tween 80 in PBS)). The animals were dosed via tail vein a single time (administration volume: 20 mL/kg; bolus injection period: about 4 minutes; recovery period: 7 days).

| | | Administration | Administration | | | |
|---|---|---|---|---|---|---|
| | Test | dosage | volume | Concentration | Number of animal | |
| Group | compound | (mg/kg) | (mL/kg) | (mg/mL) | Toxicology | Toxicokinetics |
| 1 | 5% DMSO + 95% | 0 | 20 | 0 | 3♀/3♂ | — |
| | (3% Tween | | | | | |
| | 80 in PBS) | | | | | |
| 2 | Control | 6 | 20 | 0.3 | 3♀/3♂ | 3♀/3♂ |
| 3 | compound | 20 | 20 | 1 | 3♀/3♂ | 3♀/3♂ |
| 4 | | 60 | 20 | 3 | 3♀/3♂ | 3♀/3♂ |
| 5 | Compound 6 | 6 | 20 | 0.3 | 3♀/3♂ | 3♀/3♂ |
| 6 | | 20 | 20 | 1 | 3♀/3♂ | 3♀/3♂ |
| 7 | | 60 | 20 | 3 | 3♀/3♂ | 3♀/3♂ |

The experimental results are shown in Table 7 and Table 8:

Table 7 Effect of Compound 6 and Control Compound on PLT in SD Rats after Single Intravenous Injection

| | | Compound | | Control | | Compound | | Control | |
|---|---|---|---|---|---|---|---|---|---|
| Detection | | 6 (♂) | | compound (♂) | | 6 (♀) | | compound (♀) | |
| Time | | 6 | 20 | 6 | 20 | 6 | 20 | 6 | 20 |
| point | | mg/kg | mg/kg | mg/kg | mg/kg | mg/kg | mg/kg | mg/kg | mg/kg |
| 4 | h | ↓16% | ↓51% | ↓26% | ↓81% | — | ↓61% | ↓24% | ↓89% |
| D | 2 | ↓56% | ↓71% | ↓69% | ↓91% | ↓60% | ↓84% | ↓78% | ↓96% |

— represents that no significant change was observed

TABLE 8
Effect of compound 6 and control compound on PLT
in SD rats after single intravenous injection

| Detection | Compound | Compound | Control | Control |
|---|---|---|---|---|
| time | 6 | 6 | compound | compound |
| point | (♂) | (♀) | (♂) | (♀) |
| Administration | 60 mg/kg | 60 mg/kg | 60 mg/kg | 60 mg/kg |
| dosage | | | | |
| D8 | ↓17% | ↓53% | ↓73% | ↓87% |

D2: day 2;
D8: day 8

Conclusion: at 4 h and D2 after the administration via single intravenous injection, compound 6 exhibited less platelet toxicity in rats at doses of 6 mg/kg and 20 mg/kg relative to the control compound. At D8, compound 6 exhibited less platelet toxicity in rats at the dose of 60 mg/kg relative to the control compound.

1. A compound or a stereoisomer, deuterated compound, solvate, prodrug, metabolite, pharmaceutically acceptable salt, or co-crystal thereof, wherein the compound is selected from a compound of general formula (I),
B-L-K (I);
L is selected from a bond or —C₁₋₅₀ hydrocarbyl-, wherein the hydrocarbyl has 0 to 20 methylene units optionally further replaced by -Ak- or -Cy-;
each -Ak- is independently selected from —(CH₂)_{q}—, —(CH₂)_{q}—O—, —O—(CH₂)_{q}—, —(CH₂)_{q}—NR^{L}, —NR^{L}—(CH₂)_{q}—, —(CH₂)_{q}—NR^{L}C(═O)—, —NR^{L}(CH₂)_{q}C(═O)—, —(CH₂)_{q}—C(═O)NR^{L}, —C(═O)—, —C(═O)—(CH₂)_{q}—NR^{L}—, —(C≡C)_{q}—, —CH═CH—, —Si(R^{L})₂—, —Si(OH)(R^{L})—, —Si(OH)₂—, —P(═O)(OR^{L})—, —P(═O)(R^{L})—, —S—, —S(═O)—, —S(═O)₂— or a bond, wherein the —CH₂— is optionally further substituted with 0 to 2 substituents selected from H, halogen, OH, CN, NH₂, C₁₋₆ alkyl, C₁₋₆ alkoxy, halogen-substituted C₁₋₆ alkyl, hydroxyl-substituted C₁₋₆ alkyl, or cyano-substituted C₁₋₆ alkyl;
each q is independently selected from 0, 1, 2, 3, 4, 5 or 6;
each R^{L} is independently selected from H, C₁₋₆ alkyl, 3- to 7-membered heterocyclyl, 3- to 7-membered cycloalkyl, phenyl or 5- to 6-membered heteroaryl;
each -Cy- is independently selected from a bond, a 4- to 8-membered mono-heterocyclic ring, a 4- to 10-membered fused-heterocyclic ring, a 5- to 12-membered spiro-heterocyclic ring, a 7- to 10-membered bridged-heterocyclic ring, 3- to 7-membered monocycloalkyl, 4- to 10-membered fused cycloalkyl, 5- to 12-membered spiro cycloalkyl, 7- to 10-membered bridged cycloalkyl, 5- to 10-membered heteroaryl or 6- to 10-membered aryl, wherein the aryl, heteroaryl, cycloalkyl, mono-heterocyclic ring, fused-heterocyclic ring, spiro-heterocyclic ring or bridged-heterocyclic ring is optionally further substituted with 0 to 4 substituents selected from H, F, Cl, Br, I, OH, COOH, CN, NH₂, ═O, C₁₋₄ alkyl, halogen-substituted C₁₋₄ alkyl, hydroxyl-substituted C₁₋₄ alkyl or C₁₋₄ alkoxy, and the heteroaryl, mono-heterocyclic ring, fused-heterocyclic ring, spiro-heterocyclic ring or bridged-heterocyclic ring contains 1 to 4 heteroatoms selected from O, S or N, and is optionally further substituted with 0, 1 or 2 ═O when the heteroatom is selected from S;
B is selected from 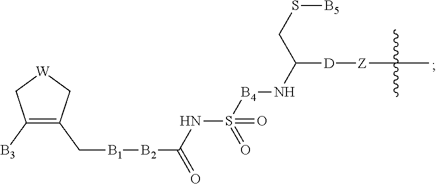
W is selected from W₁ or W₂;
W₁ is selected from —CR^{w1}R^{w2}—, —(CR^{w1}R^{w2})₃—, —(CR^{w1}R^{w2})₄—, —CH₂CR^{w3}R^{w4}—, —CR^{w3}R^{w4}CH₂—, —CR^{w1}R^{w2}O—, —OCR^{w1}R^{w2}—, —CR^{w1}R^{w2}NR^{w5}—, or —NR^{w5}CR^{w1}R^{w2}—;
W₂ is selected from —(CR^{w1}R^{w2})₂—;
D is selected from C₁₋₄ alkylene;
B₁ and Z are each independently selected from a 4- to 7-membered mono-heterocyclic ring, a 5- to 12-membered fused-heterocyclic ring, a 6- to 12-membered spiro-heterocyclic ring, or a 7- to 12-membered bridged-heterocyclic ring, the B₁ is optionally further substituted with 0 to 4 R^{B1}, and the Z is optionally further substituted with 0 to 4 R^{Q}, wherein the fused-heterocyclic ring, spiro-heterocyclic ring, or bridged-heterocyclic ring contains 1 to 3 heteroatoms selected from O, S or N, and is optionally further substituted with 0, 1 or 2 ═O when the heteroatom is selected from S;
B₂, B₃, B₄, and B₅ are each independently selected from C₆₋₁₀ aryl or 5- to 10-membered heteroaryl, the B₂ is optionally further substituted with 0 to 4 R^{B2}, the B₃ is optionally further substituted with 0 to 5 R^{B3}, the B₄ is optionally further substituted with 0 to 4 R^{B4}, and the B₅ is optionally further substituted with 0 to 5 R^{B5}, wherein the heteroaryl contains 1 to 3 heteroatoms selected from O, S or N;
R^{B1}, R^{Q}, R^{B2}, R^{B3}, and R^{B5} are each independently selected from halogen, OH, oxo, CN, C₁₋₄ alkyl or C₁₋₄ alkoxy, wherein the alkyl or alkoxy is optionally further substituted with 0 to 4 substituents selected from H, halogen, OH, CN or C₁₋₄ alkyl;
each R^{B4} is independently selected from —SO₂—C₁₋₄ alkyl, nitro, halogen, CN, OH, C₁₋₄ alkyl or C₁₋₄ alkoxy, wherein the alkyl or alkoxy is optionally further substituted with 0 to 4 substituents selected from H, halogen, OH, CN or C₁₋₄ alkyl;
R^{w1}, R^{w2} and R^{w5} are each independently selected from H, halogen, CN, OH, C₁₋₄ alkyl or C₁₋₄ alkoxy, wherein the alkyl or alkoxy is optionally further substituted with 0 to 4 substituents selected from H, halogen, OH, CN or C₁₋₄ alkyl;
alternatively, R^{w1} and R^{w2} are directly connected to form C₃₋₆ carbocycle or 3- to 6-membered heterocycle, wherein the carbocycle or heterocycle is optionally further substituted with 0 to 4 substituents selected from H, halogen, OH, CN or C₁₋₄ alkyl, and the heterocycle contains 1 to 3 heteroatoms selected from O, S or N;
R^{w3} and R^{w4} are directly connected to form C₃₋₆ carbocycle or 3- to 6-membered heterocycle, wherein the carbocycle or heterocycle is optionally further substituted with 0 to 4 substituents selected from H, halogen, OH, CN or C₁₋₄ alkyl, and the heterocycle contains 1 to 3 heteroatoms selected from O, S or N;
alternatively, R^{B1} and R^{B2} are directly connected to form C₅₋₇ carbocycle or 5- to 7-membered heterocycle, wherein the carbocycle or heterocycle is optionally further substituted with 0 to 3 R^{c}, and the heterocycle contains 1 to 3 heteroatoms selected from O, S or N;
each R^{c} is independently selected from halogen, OH, CN, ═O, C₁₋₄ alkyl, C₁₋₄ alkoxy, C₃₋₆ carbocycle or 3- to 7-membered heterocycle, wherein the alkyl, alkoxy, carbocycle or heterocycle is optionally further substituted with 0 to 4 substituents selected from H, halogen, OH, CN, C₁₋₄ alkyl, C₁₋₄ alkoxy, C₃₋₆ cycloalkyl or 3- to 7-membered heterocycloalkyl, and the heterocycle or heterocycloalkyl contains 1 to 3 heteroatoms selected from O, S or N,
provided that when W₂ is selected from —(CR^{w1}R^{w2})₂—, and R^{w1} and R^{w2} are each independently selected from F, methyl or methoxy, B at least satisfies any one of the following conditions:
1) B₁ is not piperazine;
2) Z is not piperazine, piperidine, 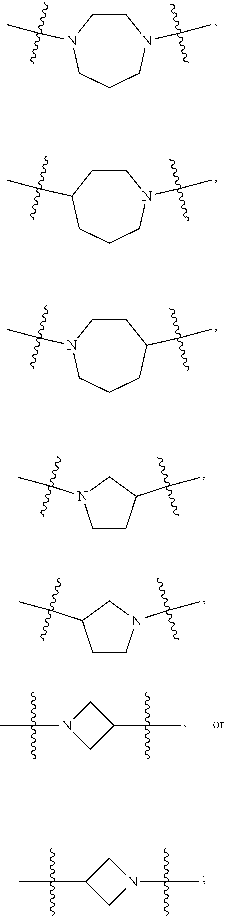
3) B₃ is selected from phenyl substituted with 1 R^{B3}, when R^{B3} is at the para-position of the phenyl, R^{B3} is not halogen, methyl or trifluoromethyl;
4) when B₂ is selected from phenyl, the phenyl is substituted with 1 to 4 R^{B2};
5) R^{B1} and R^{B2} are directly connected to form C₅₋₇ carbocycle or 5- to 7-membered heterocycle, wherein the carbocycle or heterocycle is optionally further substituted with 0 to 3 R^{c}, and the heterocycle contains 1 to 3 heteroatoms selected from O, S or N;
6) when B₅ is selected from phenyl, the phenyl is substituted with 1 to 4 R^{B5};
K is selected from 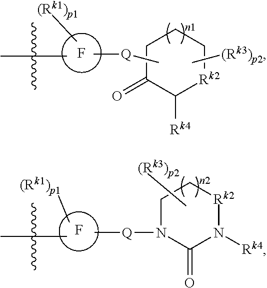
each Q is independently selected from a bond, —O—, —S—, —CH₂—, —NR^{q}—, —CO—, —NR^{q}CO—, —CONR^{q}— or 3- to 12-membered heterocyclyl, wherein the heterocyclyl is optionally further substituted with 0 to 4 substituents selected from H, F, Cl, Br, I, OH, ═O, NH₂, CN, COOH, CONH₂, C₁₋₄ alkyl or C₁₋₄ alkoxy, and the heterocyclyl contains 1 to 4 heteroatoms selected from O, S or N;
R^{q} is selected from H or C₁₋₆ alkyl;
A is selected from C₃₋₁₀ carbocyclyl, C₆₋₁₀ aryl, 3- to 10-membered heterocyclyl or 5- to 10-membered heteroaryl, wherein the heterocyclyl or heteroaryl contains 1 to 4 heteroatoms selected from O, S or N;
each F is independently selected from C₃₋₂₀ carbocyclyl, C₆₋₂₀ aryl, 3- to 20-membered heterocyclyl or 5- to 20-membered heteroaryl, wherein the heterocyclyl or heteroaryl contains 1 to 4 heteroatoms selected from O, S or N;
each R^{k2} is independently selected from a bond, —CO—, —SO₂—, —SO— or —C(R^{k3})₂—;
each R^{k1} is independently selected from H, F, Cl, Br, I, OH, ═O, NH₂, CN, COOH, CONH₂, C₁₋₆ alkyl or C₁₋₆ alkoxy, wherein the alkyl or alkoxy is optionally further substituted with 0 to 4 substituents selected from H, F, Cl, Br, I, OH, ═O, NH₂, CN, COOH, CONH₂, C₁₋₄ alkyl or C₁₋₄ alkoxy;
each R^{k3} is independently selected from H, F, Cl, Br, I, OH, ═O, NH₂, CN, COOH, CONH₂, C₁₋₆ alkyl, C₁₋₆ alkoxy, C₃₋₈ cycloalkyl or 3- to 8-membered heterocyclyl, wherein the alkyl, alkoxy, cycloalkyl or heterocyclyl is optionally further substituted with 0 to 4 substituents selected from H, F, Cl, Br, I, OH, ═O, NH₂, CN, COOH, CONH₂, C₁₋₄ alkyl or C₁₋₄ alkoxy, and the heterocyclyl contains 1 to 4 heteroatoms selected from O, S or N;
or two R^{k3} together with the carbon atoms or ring backbones to which they are directly attached form 3- to 8-membered carbocycle or 3- to 8-membered heterocycle, wherein the carbocycle or heterocycle is optionally further substituted with 0 to 4 substituents selected from H, F, Cl, Br, I, OH, ═O, NH₂, CN, COOH, CONH₂, C₁₋₄ alkyl or C₁₋₄ alkoxy, and the heterocycle contains 1 to 4 heteroatoms selected from O, S or N;
each R^{k4} is independently selected from H, OH, NH₂, CN, CONH₂, C₁₋₆ alkyl, C₃₋₈ cycloalkyl or 3- to 8-membered heterocyclyl, wherein the alkyl, cycloalkyl or heterocyclyl is optionally further substituted with 0 to 4 substituents selected from H, F, Cl, Br, I, OH, ═O, NH₂, CN, COOH, CONH₂, C₁₋₄ alkyl or C₁₋₄ alkoxy, and the heterocyclyl contains 1 to 4 heteroatoms selected from O, S or N;
M₁ is selected from a bond, —C(═O)NH—, —NHC(═O)—, —CH₂—C(═O)NH—, —C(═O)CH₂NH—, or 5- to 6-membered heteroaryl, wherein the heteroaryl is optionally further substituted with 0 to 4 substituents selected from H, F, Cl, Br, I, OH, ═O, CF₃, NH₂, CN, C₁₋₄ alkyl, halogen-substituted C₁₋₄ alkyl, hydroxyl-substituted C₁₋₄ alkyl or C₁₋₄ alkoxy, and the heteroaryl contains 1 to 4 heteroatoms selected from O, S or N;
M₂ is selected from —NHC(═O)—C₁₋₆ alkyl, —NHC(═O)—C₃₋₆ cycloalkyl or 4- to 10-membered heterocyclyl, wherein the alkyl, cycloalkyl or heterocyclyl is optionally further substituted with 0 to 4 substituents selected from H, F, Cl, Br, I, ═O, OH, NH₂, C₁₋₄ alkyl or C₁₋₄ alkoxy, and the heterocyclyl contains 1 to 4 heteroatoms selected from O, S or N;
M₃ is selected from —NH— or —O—;
R^{k10} is selected from C₁₋₆ alkyl, wherein the alkyl is optionally further substituted with 0 to 4 substituents selected from H, F, Cl, Br, I, ═O, OH, C₁₋₆ alkyl or C₃₋₆ cycloalkyl;
each R^{k11} is independently selected from H, F, Cl, Br, I, ═O, OH, SH, C₁₋₆ alkyl, C₁₋₆ alkoxy, C₁₋₆ alkylthio or —O—C(═O)—C₁₋₆ alkyl, wherein the alkyl, alkoxy or alkylthio is optionally further substituted with 0 to 4 substituents selected from H, F, Cl, Br, I, OH, C₁₋₄ alkyl or C₁₋₄ alkoxy;
R^{k12} and R^{k13} are each independently selected from H, C₁₋₆ alkyl or C₃₋₆ cycloalkyl, wherein the alkyl or cycloalkyl is optionally further substituted with 0 to 4 substituents selected from H, F, Cl, Br, I, ═O, OH, NH₂, C₁₋₄ alkyl or C₁₋₄ alkoxy;
R^{k14} is selected from 5- to 6-membered heteroaryl, wherein the heteroaryl is optionally further substituted with 0 to 4 substituents selected from H, F, Cl, Br, I, OH, ═O, CF₃, CN, C₁₋₄ alkyl, halogen-substituted C₁₋₄ alkyl, hydroxyl-substituted C₁₋₄ alkyl, C₁₋₄ alkoxy or C₃₋₆ cycloalkyl, and the heteroaryl contains 1 to 4 heteroatoms selected from N, O or S;
G is selected from 6- to 10-membered aryl or 5- to 10-membered heteroaryl, wherein the aryl or heteroaryl is optionally further substituted with 0 to 4 substituents selected from H, F, Cl, Br, I, OH, ═O, CF₃, CN, C₁₋₄ alkyl, halogen-substituted C₁₋₄ alkyl, hydroxyl-substituted C₁₋₄ alkyl, C₁₋₄ alkoxy or C₃₋₆ cycloalkyl, and the heteroaryl contains 1 to 4 heteroatoms selected from N, O or S;
optionally, 1 to 20 H of the compound of general formula (I) are replaced by 1 to 20 deuterium;
n1, n2 and n3 are each independently selected from 0, 1, 2 or 3;
each p1 or p2 is independently selected from 0, 1, 2, 3, 4 or 5.

2. The compound or the stereoisomer, deuterated compound, solvate, prodrug, metabolite, pharmaceutically acceptable salt, or co-crystal thereof according to claim 1, wherein,
L is selected from -Cy1-Ak1-Cy2-Ak2-Cy3-Ak3-Cy4-Ak4-Cy5-Ak5-, -Cy1-Cy2-Cy3-Cy4-Ak1-Ak2-Ak3-Ak4-Ak5-, -Cy1-Ak1-Cy2-Ak2-Cy3-Ak3-Cy4-Ak4-Ak5-, -Ak1-Cy1-Ak2-Cy2-Ak3-Cy3-Ak4-Cy4-Ak5-, -Cy1-Ak1-Cy2-Ak2-Cy3-Cy4-Ak3-Ak4-Ak5-, -Cy1-Ak1-Cy2-Ak2-Ak3-Cy3-Cy4-Ak4-Ak5-, -Cy1-Ak1-Ak2-Ak3-Ak4-Ak5-Cy2-Cy3-Cy4-, -Cy1-Cy2-Ak1-Ak2-Ak3-Ak4-Ak5-Cy3-Cy4-, -Cy1-Cy2-Cy3-Ak1-Ak2-Ak3-Ak4-Ak5-Cy4-, -Cy1-Cy2-Cy3-Cy4-Ak1-Ak2-Ak3-Ak4-Ak5-, -Cy1-Ak1-Cy2-Cy3-Cy4-Ak2-Ak3-Ak4-Ak5-, -Cy1-Cy2-Ak1-Cy3-Cy4-Ak2-Ak3-Ak4-Ak5-, -Cy1-Cy2-Cy3-Ak1-Cy4-Ak2-Ak3-Ak4-Ak5-, -Cy1-Ak1-Ak2-Cy2-Cy3-Cy4-Ak3-Ak4-Ak5-, -Cy1-Cy2-Ak1-Ak2-Cy3-Cy4-Ak3-Ak4-Ak5-, -Cy1-Cy2-Cy3-Ak1-Ak2-Cy4-Ak3-Ak4-Ak5-, -Cy1-Ak1-Ak2-Ak3-Cy2-Cy3-Cy4-Ak4-Ak5-, -Cy1-Cy2-Ak1-Ak2-Ak3-Cy3-Cy4-Ak4-Ak5-, -Cy1-Cy2-Cy3-Ak1-Ak2-Ak3-Cy4-Ak4-Ak5-, -Cy1-Ak1-Ak2-Ak3-Ak4-Cy2-Cy3-Cy4-Ak5-, -Cy1-Cy2-Ak1-Ak2-Ak3-Ak4-Cy3-Cy4-Ak5-, -Cy1-Cy2-Cy3-Ak1-Ak2-Ak3-Ak4-Cy4-Ak5-, -Ak1-Ak2-Ak3-Ak4-Ak5-Cy1-Cy2-Cy3-Cy4-, -Ak1-Cy1-Cy2-Cy3-Cy4-Ak2-Ak3-Ak4-Ak5-, -Ak1-Ak2-Cy1-Cy2-Cy3-Cy4-Ak3-Ak4-Ak5-, -Ak1-Ak2-Ak3-Cy1-Cy2-Cy3-Cy4-Ak4-Ak5-, -Ak1-Ak2-Ak3-Ak4-Cy1-Cy2-Cy3-Cy4-Ak5-, -Ak1-Cy1-Ak2-Ak3-Ak4-Ak5-Cy2-Cy3-Cy4-, -Ak1-Cy1-Cy2-Ak2-Ak3-Ak4-Ak5-Cy3-Cy4-, -Ak1-Cy1-Cy2-Cy3-Ak2-Ak3-Ak4-Ak5-Cy4-, -Ak1-Ak2-Cy1-Ak3-Ak4-Ak5-Cy2-Cy3-Cy4-, -Ak1-Ak2-Cy1-Cy2-Ak3-Ak4-Ak5-Cy3-Cy4-, -Ak1-Ak2-Cy1-Cy2-Cy3-Ak3-Ak4-Ak5-Cy4-, -Ak1-Ak2-Ak3-Cy1-Ak4-Ak5-Cy2-Cy3-Cy4-, -Ak1-Ak2-Ak3-Cy1-Cy2-Ak4-Ak5-Cy3-Cy4-, -Ak1-Ak2-Ak3-Cy1-Cy2-Cy3-Ak4-Ak5-Cy4-, -Ak1-Ak2-Ak3-Ak4-Cy1-Ak5-Cy2-Cy3-Cy4-, -Ak1-Ak2-Ak3-Ak4-Cy1-Cy2-Ak5-Cy3-Cy4-, -Ak1-Ak2-Ak3-Ak4-Cy1-Cy2-Cy3-Ak5-Cy4-, -Ak1-, -Ak1-Ak2-, -Ak1-Ak2-Ak3-, -Ak1-Ak2-Ak3-Ak4-, -Ak1-Ak2-Ak3-Ak4-Ak5-, -Ak1-Ak2-Ak3-Ak4-Ak5-Ak6-, -Ak1-Ak2-Ak3-Ak4-Ak5-Ak6-Ak7-, -Ak1-Ak2-Ak3-Ak4-Ak5-Ak6-Ak7-Ak8-, or -Ak1-Ak2-Ak3-Ak4-Ak5-Ak6-Ak7-Ak8-Ak9;
Ak1, Ak2, Ak3, Ak4, Ak5, Ak6, Ak7, Ak8 and Ak9 are each independently selected from —(CH₂)_{q}—, —(CH₂)_{q}—O—, —O—(CH₂)_{q}—, —(CH₂)_{q}—NR^{L}—, —NR^{L}—(CH₂)_{q}—, —(CH₂)_{q}—NR^{L}C(═O)—, —(CH₂)_{q}—C(═O)NR^{L}—, —C(═O)—, —C(═O)—(CH₂)_{q}—NR^{L}—, —(C≡C)_{q}— or a bond, wherein the —CH₂— is optionally further substituted with 0 to 2 substituents selected from H, halogen, OH, CN, NH₂, C₁₋₄ alkyl, Ci-4 alkoxy, halogen-substituted C₁₋₄ alkyl, hydroxyl-substituted C₁₋₄ alkyl or cyano-substituted C₁₋₄ alkyl;
each Cy1, Cy2, Cy3, Cy4 or Cy5 is independently selected from a bond, a 4- to 7-membered mono-heterocyclic ring, a 4- to 10-membered fused-heterocyclic ring, a 5- to 12-membered spiro-heterocyclic ring, a 7- to 10-membered bridged-heterocyclic ring, 3- to 7-membered monocycloalkyl, 4- to 10-membered fused cycloalkyl, 5- to 12-membered spiro cycloalkyl, 7- to 10-membered bridged cycloalkyl, 5- to 10-membered heteroaryl or 6- to 10-membered aryl, wherein the aryl, heteroaryl, cycloalkyl, mono-heterocyclic ring, fused-heterocyclic ring, spiro-heterocyclic ring or bridged-heterocyclic ring is optionally further substituted with 0 to 4 substituents selected from H, F, Cl, Br, I, OH, COOH, CN, NH₂, ═O, C₁₋₄ alkyl, halogen-substituted C₁₋₄ alkyl, hydroxyl-substituted C₁₋₄ alkyl or C₁₋₄ alkoxy, and the heteroaryl, mono-heterocyclic ring, fused-heterocyclic ring, spiro-heterocyclic ring or bridged-heterocyclic ring contains 1 to 4 heteroatoms selected from O, S or N, and is optionally further substituted with 0, 1 or 2 ═O when the heteroatom is selected from S;
each q is independently selected from 0, 1, 2, 3 or 4;
each R^{L} is independently selected from H or C₁₋₆ alkyl.

3. The compound or the stereoisomer, deuterated compound, solvate, prodrug, metabolite, pharmaceutically acceptable salt, or co-crystal thereof according to claim 2, wherein,
Ak1, Ak2, Ak3, Ak4, Ak5, Ak6, Ak7, Ak8 and Ak9 are each independently selected from —(CH₂)_{q}—, —(CH₂)_{q}—O—, —O—(CH₂)_{q}—, —(CH₂)_{q}—NR^{L}—, —NR^{L}—(CH₂)_{q}—, —(CH₂)_{q}—NR^{L}C(═O)—, —(CH₂)_{q}—C(═O)NR^{L}—, —C(═O)—, —C(═O)—(CH₂)_{q}—NR^{L}—, —(C≡C)_{q}—, or a bond, wherein the —CH₂— is optionally further substituted with 0 to 2 substituents selected from H, F, Cl, Br, I, OH, CN, NH₂, CF₃, hydroxymethyl, C₁₋₄ alkyl, or C₁₋₄ alkoxy;
each Cy1, Cy2, Cy3, Cy4 or Cy5 is independently selected from a bond, a 4- to 7-membered nitrogen-containing mono-heterocyclic ring, a 4- to 10-membered nitrogen-containing fused-heterocyclic ring, a 5- to 12-membered nitrogen-containing spiro-heterocyclic ring, a 7- to 10-membered nitrogen-containing bridged-heterocyclic ring, 3- to 7-membered monocycloalkyl, 4- to 10-membered fused cycloalkyl, 5- to 12-membered spiro cycloalkyl, 7- to 10-membered bridged cycloalkyl, 5- to 10-membered heteroaryl or 6- to 10-membered aryl, wherein the mono-heterocyclic ring, fused-heterocyclic ring, bridged-heterocyclic ring, spiro-heterocyclic ring, cycloalkyl, aryl or heteroaryl is optionally further substituted with 0 to 4 substituents selected from H, F, Cl, Br, I, OH, COOH, CN, NH₂, ═O, C₁₋₄ alkyl, halogen-substituted C₁₋₄ alkyl, hydroxyl-substituted C₁₋₄ alkyl or C₁₋₄ alkoxy, and the mono-heterocyclic ring, fused-heterocyclic ring, bridged-heterocyclic ring, spiro-heterocyclic ring or heteroaryl contains 1 to 4 heteroatoms selected from O, S or N, and is optionally further substituted with 0, 1 or 2 ═O when the heteroatom is selected from S;
each R^{L} is independently selected from H or C₁₋₄ alkyl;
K is selected from 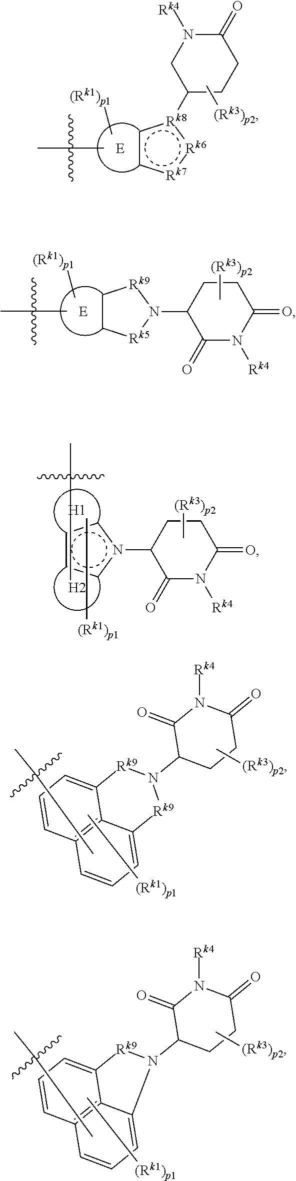 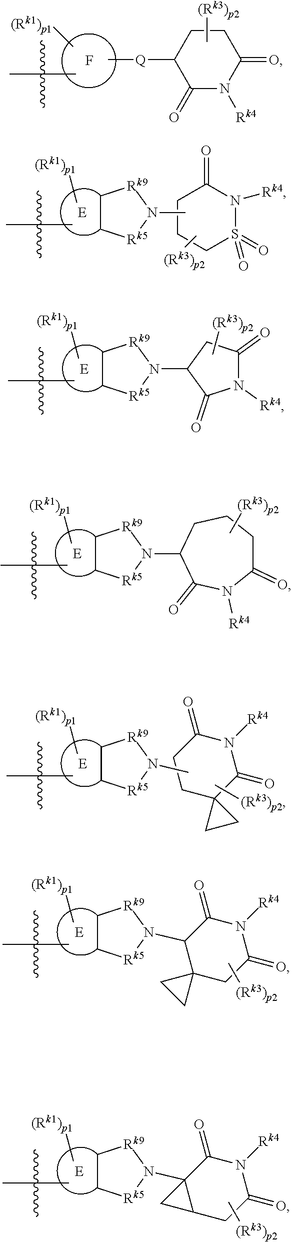 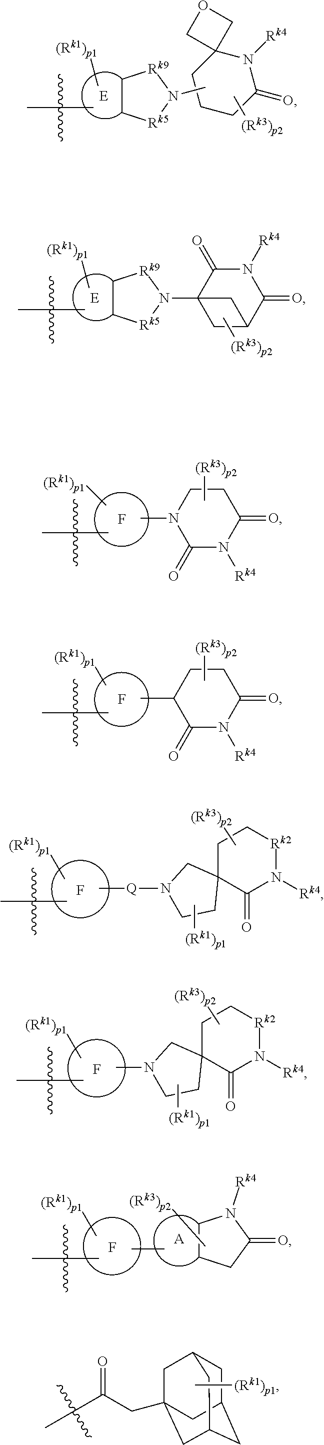 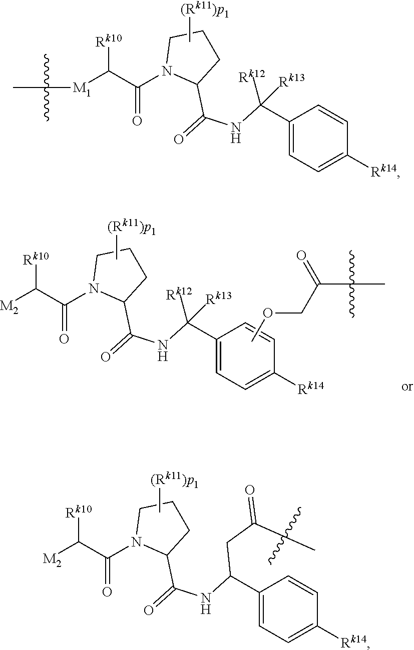 [symbol]
represents a ring selected from an aromatic ring or a non-aromatic ring;
M₂ is selected from —NHC(═O)—C₁₋₄ alkyl, —NHC(═O)—C₃₋₆ cycloalkyl or 4- to 10-membered heterocyclyl, wherein the alkyl, cycloalkyl or heterocyclyl is optionally further substituted with 0 to 4 substituents selected from H, F, Cl, Br, I, ═O, OH, NH₂, C₁₋₄ alkyl or C₁₋₄ alkoxy, and the heterocyclyl contains 1 to 4 heteroatoms selected from O, S or N;
R^{k10} is selected from C₁₋₄ alkyl, wherein the alkyl is optionally further substituted with 0 to 4 substituents selected from H, F, Cl, Br, I, ═O, OH, C₁₋₄ alkyl or C₃₋₆ cycloalkyl;
each R^{k11} is independently selected from H, F, Cl, Br, I, ═O, OH, SH, C₁₋₄ alkyl, C₁₋₄ alkoxy or C₁₋₄ alkylthio or —O—C(═O)—C₁₋₄ alkyl, wherein the alkyl, alkoxy or alkylthio is optionally further substituted with 0 to 4 substituents selected from H, F, Cl, Br, I, OH, C₁₋₄ alkyl or C₁₋₄ alkoxy;
R^{k12} and R^{k13} are each independently selected from H, C₁₋₄ alkyl or C₃₋₆ cycloalkyl, wherein the alkyl or cycloalkyl is optionally further substituted with 0 to 4 substituents selected from H, F, Cl, Br, I, ═O, OH, NH₂, C₁₋₄ alkyl or C₁₋₄ alkoxy;
each Q is independently selected from —O—, —S—, —CH₂—, —NR^{q}—, —CO—, —NR^{q}CO—, —CONR^{q}— or 4- to 7-membered heterocyclyl, wherein the heterocyclyl is optionally further substituted with 0 to 4 substituents selected from H, F, Cl, Br, I, OH, ═O, NH₂, CN, COOH, CONH₂, C₁₋₄ alkyl or C₁₋₄ alkoxy, and the heterocyclyl contains 1 to 4 heteroatoms selected from O, S or N;
R^{q} is selected from H or C₁₋₄ alkyl;
R^{k1} and R^{k3} are each independently selected from H, F, Cl, Br, I, OH, ═O, NH₂, CF₃, CN, COOH, CONH₂, C₁₋₄ alkyl or C₁₋₄ alkoxy, wherein the alkyl or alkoxy is optionally further substituted with 0 to 4 substituents selected from H, F, Cl, Br, I, OH, or NH₂;
or two R^{k3} together with the carbon atoms or ring backbones to which they are directly attached form 3- to 6-membered carbocycle or 3- to 7-membered heterocycle, wherein the carbocycle or heterocycle is optionally further substituted with 0 to 4 substituents selected from H, F, Cl, Br, I, OH, ═O, NH₂, CN, COOH, CONH₂, C₁₋₄ alkyl or C₁₋₄ alkoxy, and the heterocycle contains 1 to 4 heteroatoms selected from O, S or N;
each R^{k4} is independently selected from H, OH, NH₂, CF₃, CN or C₁₋₄ alkyl;
each R^{k5} is independently selected from CO, CH₂, SO₂ or 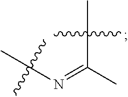 [SMILES: */N=C(\*)C]
each R^{k6} is independently selected from CO, CH, SO, SO₂, CH₂ or N;
each R^{k7} is independently selected from CO, CH, N, CH₂, O, S, N(CH₃) or NH;
each R^{k8} is independently selected from C, N or CH;
each R^{k9} is independently selected from CO, CH₂ or SO₂;
each A, H1 or H2 is independently selected from C₃₋₈ carbocycle, a benzene ring, 4- to 7-membered heterocycle or 5- to 6-membered heteroaryl, wherein the heterocycle or heteroaryl contains 1 to 4 heteroatoms selected from O, S or N;
each E is independently selected from C₃₋₈ carbocycle, a benzene ring, 4- to 7-membered heterocycle, 8- to 12-membered heterocycle, 7- to 12-membered heteroaryl or 5- to 6-membered heteroaryl, wherein the heterocycle or heteroaryl contains 1 to 4 heteroatoms selected from O, S or N;
each F is independently selected from 3- to 7-membered monocycloalkyl, 4- to 10-membered fused cycloalkyl, 5- to 12-membered spiro cycloalkyl, 5- to 10-membered bridged cycloalkyl, a 4- to 7-membered mono-heterocyclic ring, a 4- to 10-membered fused-heterocyclic ring, a 5- to 12-membered spiro-heterocyclic ring, a 5- to 10-membered bridged-heterocyclic ring, C₆₋₁₄ aryl or 5- to 10-membered heteroaryl, wherein the mono-heterocyclic ring, fused-heterocyclic ring, spiro-heterocyclic ring, bridged-heterocyclic ring or heteroaryl contains 1 to 4 heteroatoms selected from O, S or N.

4. The compound or the stereoisomer, deuterated compound, solvate, prodrug, metabolite, pharmaceutically acceptable salt, or co-crystal thereof according to claim 3, wherein,
Ak1, Ak2, Ak3, Ak4, Ak5, Ak6, Ak7, Ak8 and Ak9 are each independently selected from —(CH₂)_{q}—, —(CH₂)_{q}—O—, —O—(CH₂)_{q}—, —(CH₂)_{q}—NR^{L}—, —NR^{L}—(CH₂)_{q}—, —(CH₂)_{q}—NR^{L}C(═O)—, —(CH₂)_{q}—C(═O)NR^{L}—, —C(═O)—, —C(═O)—(CH₂)_{q}—NR^{L}—, —(C≡C)_{q}—, or a bond, wherein the —CH₂— is optionally further substituted with 0 to 2 substituents selected from H, F, Cl, Br, I, OH, CN, NH₂, CF₃, hydroxymethyl, methyl, ethyl, methoxy or ethoxy;
R^{L} is selected from H, methyl or ethyl;
each q is independently selected from 0, 1, 2 or 3;
each Cy1, Cy2, Cy3, Cy4 or Cy5 is independently selected from a bond or one of the following substituted or unsubstituted groups: cyclopropyl, cyclobutyl, cyclopentyl, cyclohexyl, azetidinyl, azacyclopentyl, azacyclohexenyl, piperidyl, morpholinyl, piperazinyl, phenyl, cyclopropyl-fused-cyclopropyl, cyclopropyl-fused-cyclobutyl, cyclopropyl-fused-cyclopentyl, cyclopropyl-fused-cyclohexyl, cyclobutyl-fused-cyclobutyl, cyclobutyl-fused-cyclopentyl, cyclobutyl-fused-cyclohexyl, cyclopentyl-fused-cyclopentyl, cyclopentyl-fused-cyclohexyl, cyclohexyl-fused-cyclohexyl, cyclopropyl-spiro-cyclopropyl, cyclopropyl-spiro-cyclobutyl, cyclopropyl-spiro-cyclopentyl, cyclopropyl-spiro-cyclohexyl, cyclobutyl-spiro-cyclobutyl, cyclobutyl-spiro-cyclopentyl, cyclobutyl-spiro-cyclohexyl, cyclopentyl-spiro-cyclopentyl, cyclopentyl-spiro-cyclohexyl, cyclohexyl-spiro-cyclohexyl, cyclopropyl-fused-azetidinyl, cyclopropyl-fused-azacyclopentyl, cyclopropyl-fused-azacyclohexyl, cyclopropyl-fused-piperidyl, cyclobutyl-fused-azetidinyl, cyclobutyl-fused-azacyclopentyl, cyclobutyl-fused-azacyclohexyl, cyclobutyl-fused-piperidyl, cyclopentyl-fused-azetidinyl, cyclopentyl-fused-azacyclopentyl, cyclopentyl-fused-azacyclohexyl, cyclopentyl-fused-piperidyl, cyclohexyl-fused-azetidinyl, cyclohexyl-fused-azacyclopentyl, cyclohexyl-fused-azacyclohexyl, cyclohexyl-fused-piperidyl, azetidinyl-fused-azetidinyl, azetidinyl-fused-azacyclopentyl, azetidinyl-fused-azacyclohexyl, azetidinyl-fused-piperidyl, azacyclopentyl-fused-azetidinyl, azacyclopentyl-fused-azacyclopentyl, azacyclopentyl-fused-azacyclohexyl, azacyclopentyl-fused-piperidyl, azacyclohexyl-fused-azetidinyl, azacyclohexyl-fused-azacyclopentyl, azacyclohexyl-fused-azacyclohexyl, azacyclohexyl-fused-piperidyl, cyclobutyl-spiro-azetidinyl, cyclobutyl-spiro-azacyclopentyl, cyclobutyl-spiro-azacyclohexyl, cyclopentyl-spiro-azetidinyl, cyclopentyl-spiro-azacyclopentyl, cyclopentyl-spiro-azacyclohexyl, cyclohexyl-spiro-azetidinyl, cyclohexyl-spiro-azacyclopentyl, cyclohexyl-spiro-azacyclohexyl, azetidinyl-spiro-azetidinyl, azetidinyl-spiro-azacyclopentyl, azetidinyl-spiro-azacyclohexyl, azacyclopentyl-spiro-azetidinyl, azacyclopentyl-spiro-azacyclopentyl, azacyclopentyl-spiro-azacyclohexyl, azacyclohexyl-spiro-azetidinyl, azacyclohexyl-spiro-azacyclopentyl, azacyclohexyl-spiro-azacyclohexyl, cyclobutyl-spiro-piperidyl, cyclopentyl-spiro-piperidyl, cyclohexyl-spiro-piperidyl, azetidinyl-spiro-piperidyl, azacyclopentyl-spiro-piperidyl, azacyclohexyl-spiro-piperidyl, 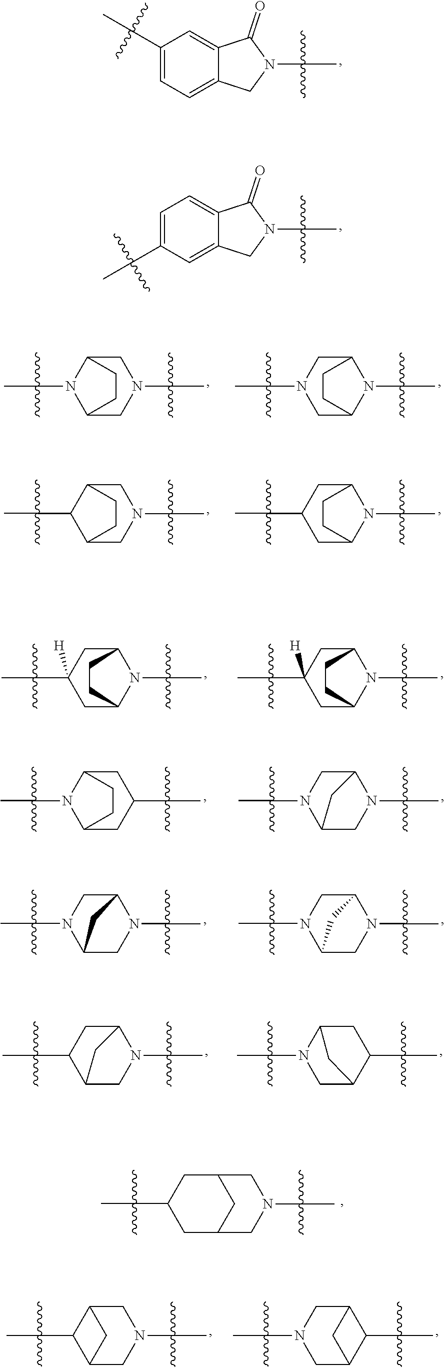 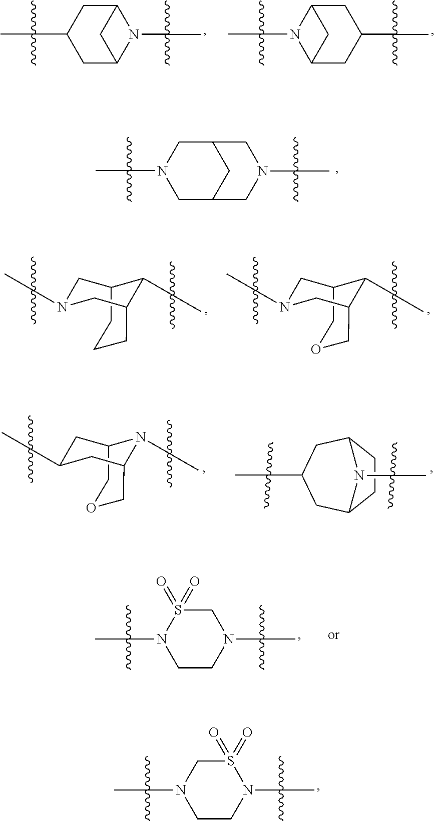
which, when substituted, is optionally further substituted with 0 to 4 substituents selected from H, F, Cl, Br, I, OH, NH₂, COOH, CN, ═O, C₁₋₄ alkyl, halogen-substituted C₁₋₄ alkyl, hydroxyl-substituted C₁₋₄ alkyl, or C₁₋₄ alkoxy;
D is selected from ethylene;
B₁ and Z are each independently selected from azetidinyl, azacyclopentyl, piperazinyl, piperidyl, azacyclohexenyl, azepanyl, 1,4-diazepanyl, cyclobutyl-fused-azetidinyl, cyclobutyl-fused-azacyclopentyl, cyclobutyl-fused-azacyclohexyl, cyclobutyl-fused-piperidyl, cyclopentyl-fused-azetidinyl, cyclopentyl-fused-azacyclopentyl, cyclopentyl-fused-azacyclohexyl, cyclopentyl-fused-piperidyl, cyclohexyl-fused-azetidinyl, cyclohexyl-fused-azacyclopentyl, cyclohexyl-fused-azacyclohexyl, cyclohexyl-fused-piperidyl, azetidinyl-fused-azetidinyl, azacyclopentyl-fused-azetidinyl, azacyclopentyl-fused-azacyclopentyl, azacyclopentyl-fused-azacyclohexyl, azacyclopentyl-fused-piperidyl, azacyclohexyl-fused-azetidinyl, azacyclohexyl-fused-azacyclopentyl, azacyclohexyl-fused-azacyclohexyl, azacyclohexyl-fused-piperidyl, cyclobutyl-spiro-azetidinyl, cyclobutyl-spiro-azacyclopentyl, cyclobutyl-spiro-azacyclohexyl, cyclopentyl-spiro-azetidinyl, cyclopentyl-spiro-azacyclopentyl, cyclopentyl-spiro-azacyclohexyl, cyclohexyl-spiro-azetidinyl, cyclohexyl-spiro-azacyclopentyl, azetidinyl-spiro-azetidinyl, azetidinyl-spiro-azacyclopentyl, azetidinyl-spiro-azacyclohexyl, azacyclopentyl-spiro-azetidinyl, azacyclopentyl-spiro-azacyclopentyl, azacyclopentyl-spiro-azacyclohexyl, azacyclohexyl-spiro-azetidinyl, azacyclohexyl-spiro-azacyclopentyl, azacyclohexyl-spiro-azacyclohexyl, cyclobutyl-spiro-piperidyl, cyclopentyl-spiro-piperidyl, cyclohexyl-spiro-piperidyl, azetidinyl-spiro-piperidyl, azacyclopentyl-spiro-piperidyl, azacyclohexyl-spiro-piperidyl, 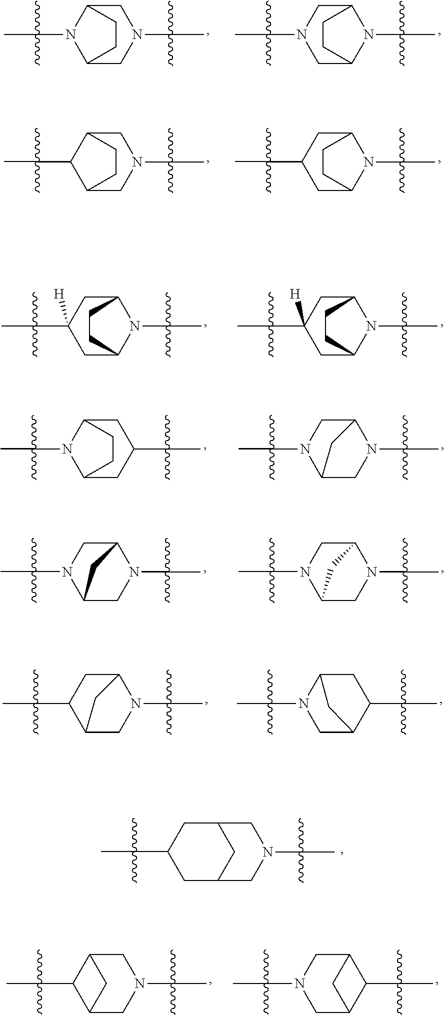 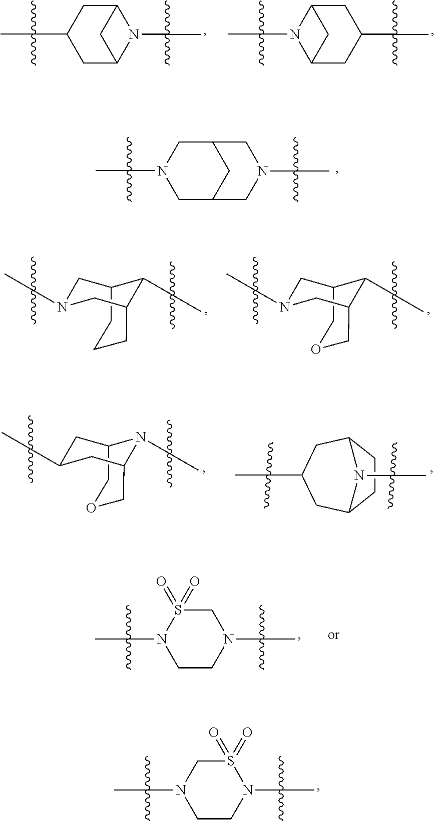
the B₁ is optionally further substituted with 0 to 4 R^{B1}, and the Z is optionally further substituted with 0 to 4 R^{Q};
B₂ and B₄ are each independently selected from phenyl or 5- to 6-membered heteroaryl, B₃ and B₅ are each independently selected from phenyl, naphthyl, 5- to 6-membered heteroaryl or benzo 5- to 6-membered heteroaryl, the B₂ is optionally further substituted with 0 to 4 R^{B2}, the B₃ is optionally further substituted with 0 to 5 R^{B3}, the B₄ is optionally further substituted with 0 to 4 R^{B4}, and the B₅ is optionally further substituted with 0 to 5 R^{B5}, wherein the heteroaryl contains 1 to 3 heteroatoms selected from O, S or N;
R^{B1}, R^{Q}, R^{B2}, R^{B3} and R^{B5} are each independently selected from F, Cl, Br, I, oxo, OH, CN, methyl, ethyl, methoxy or ethoxy, wherein the methyl, ethyl, methoxy or ethoxy is optionally further substituted with 0 to 4 substituents selected from H, halogen, OH, CN or C₁₋₄ alkyl;
each R^{B4} is independently selected from —SO₂-methyl, —SO₂-ethyl, nitro, F, Cl, Br, I, OH, CN, methyl, ethyl, methoxy or ethoxy, wherein the methyl, ethyl, methoxy or ethoxy is optionally further substituted with 0 to 4 substituents selected from H, halogen, OH, CN or C₁₋₄ alkyl;
R^{w1}, R^{w2} and R^{w5} are each independently selected from H, F, Cl, Br, I, OH, CN, methyl, ethyl, methoxy or ethoxy, wherein the methyl, ethyl, methoxy or ethoxy is optionally further substituted with 0 to 4 substituents selected from H, halogen, OH, CN or C₁₋₄ alkyl;
alternatively, R^{w1} and R^{w2} are directly connected to form C₃₋₆ carbocycle or 3- to 6-membered heterocycle, wherein the carbocycle or heterocycle is optionally further substituted with 0 to 4 substituents selected from H, halogen, OH, CN or C₁₋₄ alkyl, and the heterocycle contains 1 to 3 heteroatoms selected from O, S or N;
R^{w3} and R^{w4} are directly connected to form C₃₋₆ carbocycle or 3- to 6-membered heterocycle, wherein the carbocycle or heterocycle is optionally further substituted with 0 to 4 substituents selected from H, halogen, OH, CN or C₁₋₄ alkyl, and the heterocycle contains 1 to 3 heteroatoms selected from O, S or N;
alternatively, R^{B1} and R^{B2} are directly connected to form C₅₋₇ carbocycle or 5- to 7-membered heterocycle, wherein the carbocycle or heterocycle is optionally further substituted with 0 to 3 R^{c}, and the heterocycle contains 1 to 3 heteroatoms selected from O, S or N;
each R^{c} is independently selected from F, Cl, Br, I, OH, CN, ═O, methyl, ethyl, methoxy, ethoxy, cyclopropyl, cyclobutyl, cyclopentyl, cyclohexyl, oxetanyl, oxacyclopentyl, oxacyclohexyl, azetidinyl, azacyclopentyl, azacyclohexyl, morpholinyl, or piperazinyl, wherein the methyl, ethyl, methoxy, ethoxy, cyclopropyl, cyclobutyl, cyclopentyl, cyclohexyl, oxetanyl, oxacyclopentyl, oxacyclohexyl, azetidinyl, azacyclopentyl, azacyclohexyl, morpholinyl, or piperazinyl is optionally further substituted with 0 to 4 substituents selected from H, halogen, OH, CN, C₁₋₄ alkyl, C₁₋₄ alkoxy, C₃₋₆ cycloalkyl, or 3- to 7-membered heterocycloalkyl, and the heterocycloalkyl contains 1 to 3 heteroatoms selected from O, S or N;
K is selected from 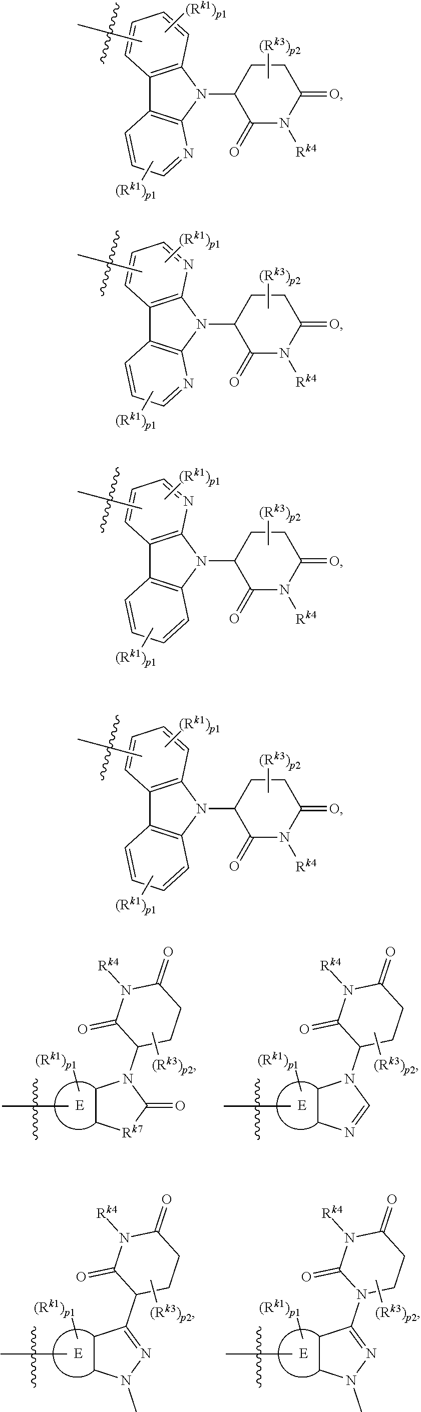 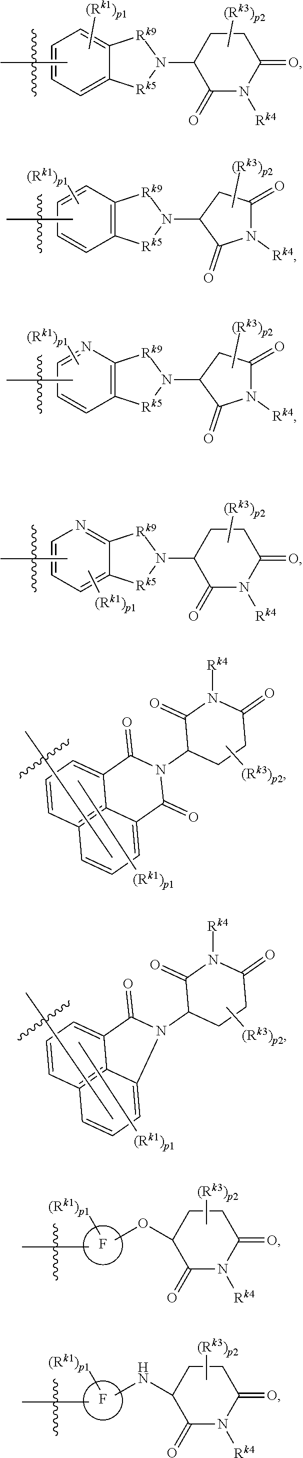 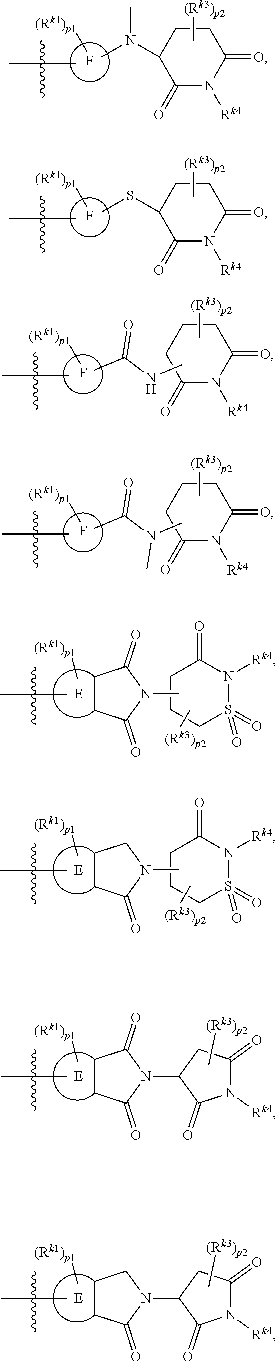 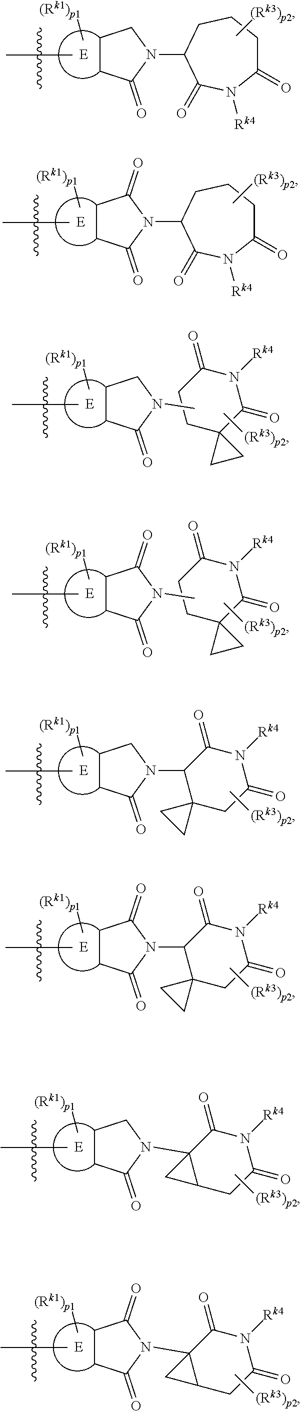 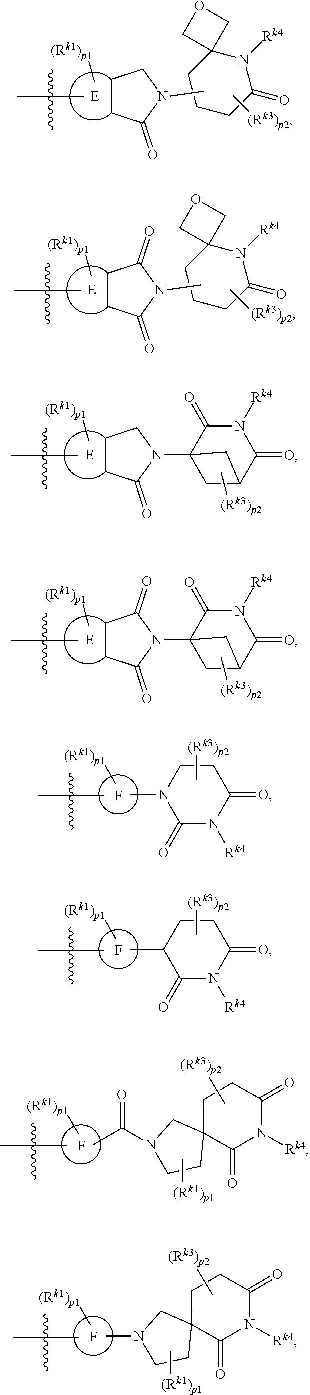 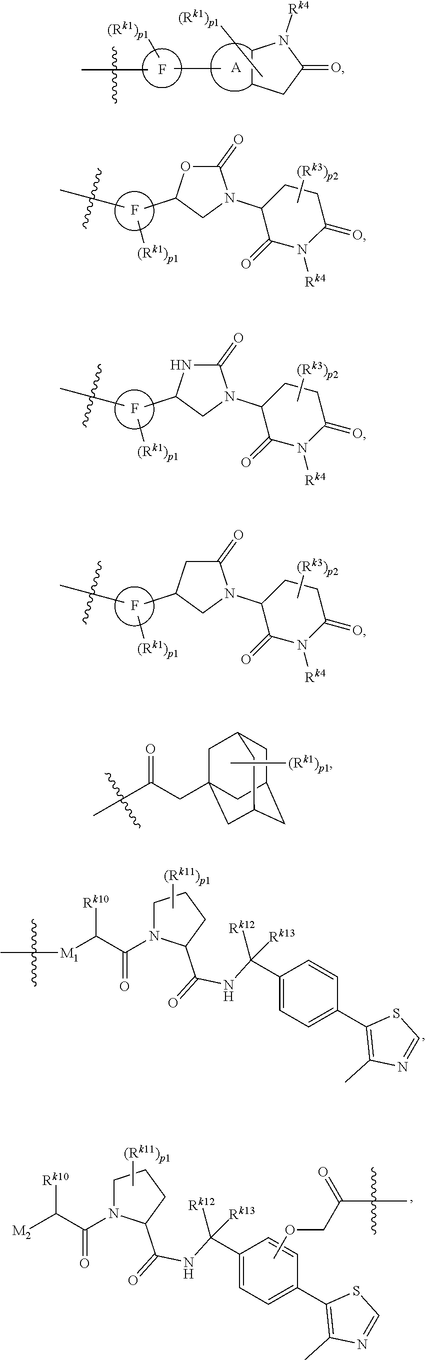 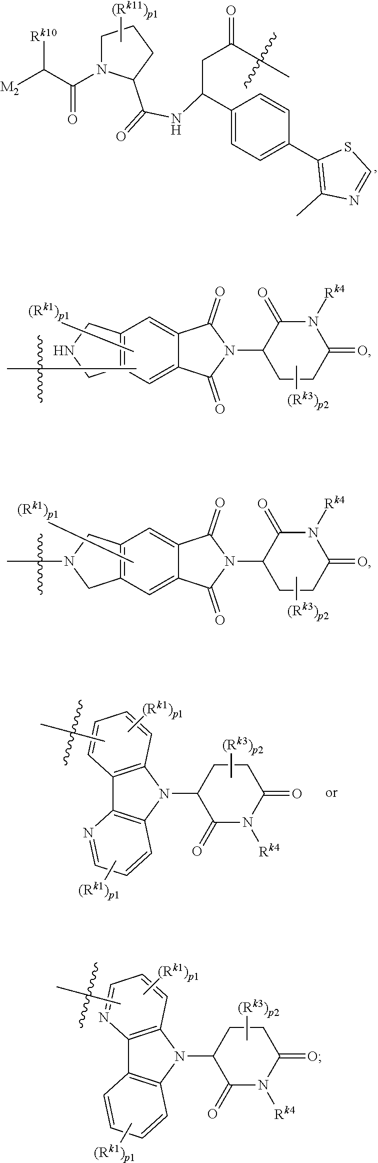
M₁ is selected from a bond, —C(═O)NH—, —CH₂—C(═O)NH—, —C(═O)CH₂NH—, pyrrolyl, pyrazolyl, imidazolyl, triazolyl, oxazolyl, isoxazolyl, furyl, thienyl or thiazolyl;
M₂ is selected from —NHC(═O)—CH₃, —NHC(═O)-cyclopropyl, —NHC(═O)-cyclobutyl, azetidinyl, azacyclopentyl, benzo-azacyclopentyl or benzo-azacyclohexyl, wherein the cyclopropyl, cyclobutyl, azetidinyl, azacyclopentyl, benzo-azacyclopentyl or benzo-azacyclohexyl is optionally further substituted with 0 to 4 substituents selected from H, F, Cl, Br, I, ═O, OH, NH₂, C₁₋₄ alkyl or C₁₋₄ alkoxy;
R^{k10} is selected from methyl, ethyl, isopropyl, propyl or tert-butyl, wherein the methyl, ethyl, isopropyl, propyl or tert-butyl is optionally further substituted with 0 to 4 substituents selected from H, F, Cl, Br, I, ═O, OH, C₁₋₄ alkyl or C₃₋₆ cycloalkyl;
each R^{k11} is independently selected from H, F, Cl, Br, I, ═O, OH, SH, methyl, ethyl, isopropyl, propyl, methoxy, ethoxy, propoxy, isopropyloxy, methylthio, ethylthio, propylthio or —O—C(═O)—CH₃, wherein the methyl, ethyl, isopropyl, propyl, methoxy, ethoxy, propoxy, isopropyloxy, methylthio, ethylthio, or propylthio is optionally further substituted with 0 to 4 substituents selected from H, F, Cl, Br, I, OH, C₁₋₄ alkyl or C₁₋₄ alkoxy;
R^{k12} and R^{k13} are each independently selected from H, methyl, ethyl, isopropyl, propyl, cyclopropyl or cyclobutyl, wherein the methyl, ethyl, isopropyl, propyl, cyclopropyl or cyclobutyl is optionally further substituted with 0 to 4 substituents selected from H, F, Cl, Br, I, ═O, OH, NH₂, C₁₋₄ alkyl or C₁₋₄ alkoxy;
each E is independently selected from phenyl, pyridyl, pyridazinyl, pyrazinyl, pyrimidyl, pyrrolyl, pyrazolyl, imidazolyl, thiazolyl, furyl, thienyl or oxazolyl;
each A is independently selected from phenyl, pyridyl, pyridazinyl, pyrazinyl, pyrimidyl, pyrrolyl, pyrazolyl, imidazolyl, thiazolyl, furyl, thienyl or oxazolyl;
each F is independently selected from cyclopropyl, cyclobutyl, cyclopentyl, cyclohexyl, bicyclo[1.1.1]pentanyl, 6,7-dihydro-5H-cyclopenta[c]pyridyl, 2,3-dihydro-1H-indenyl, phenyl, naphthyl, anthryl, phenanthryl, azetidinyl, azacyclopentyl, piperidyl, morpholinyl, pyridyl, pyrimidyl, pyridazinyl, pyrazinyl, triazinyl, pyrrolyl, pyrazolyl, imidazolyl, triazolyl, oxazolyl, furyl, thienyl, thiazolyl, benzoimidazolyl, benzopyrazolyl, benzothiazolyl, benzothienyl, benzofuryl, benzopyrrolyl, benzopyridyl, benzopyrazinyl, benzopyrimidyl, benzopyridazinyl, pyrrolopyrrolyl, pyrrolopyridyl, pyrrolopyrimidyl, pyrrolopyridazinyl, pyrrolopyrazinyl, imidazopyrimidyl, imidazopyridyl, imidazopyrazinyl, imidazopyridazinyl, pyrazolopyridyl, pyrazolopyrimidyl, pyrazolopyridazinyl, pyrazolopyrazinyl, pyrimidopyridyl, pyrimidopyrazinyl, pyrimidopyridazinyl, pyrimidopyrimidyl, pyridopyridyl, pyridopyrazinyl, pyridopyridazinyl, pyridazinopyridazinyl, pyridazinopyrazinyl or pyrazinopyrazinyl;
each R^{k7} is independently selected from CH₂, O, N(CH₃) or NH;
each p1 or p2 is independently selected from 0, 1 or 2.

5. The compound or the stereoisomer, deuterated compound, solvate, prodrug, metabolite, pharmaceutically acceptable salt, or co-crystal thereof according to claim 4, wherein,
Ak1, Ak2, Ak3, Ak4, Ak5, Ak6, Ak7, Ak8 and Ak9 are each independently selected from a bond, —O—, —OCH₂—, —CH₂O—, —OCH₂CH₂—, —CH₂CH₂O—, —C≡C—, —C(CH₃)₂—, —CH₂—, —CH₂CH₂—, —CH₂CH₂CH₂—, —N(CH₃)—, —NH—, —CH₂N(CH₃)—, —CH₂NH—, —NHCH₂—, —CH₂CH₂N(CH₃)—, —CH₂CH₂NH—, —NHCH₂CH₂—, —C(═O)—, —C(═O)CH₂NH—, —CH₂C(═O)NH—, —C(═O)NH— or —NHC(═O)—;
each Cy1, Cy2, Cy3, Cy4 or Cy5 is independently selected from a bond or one of the following substituted or unsubstituted groups: 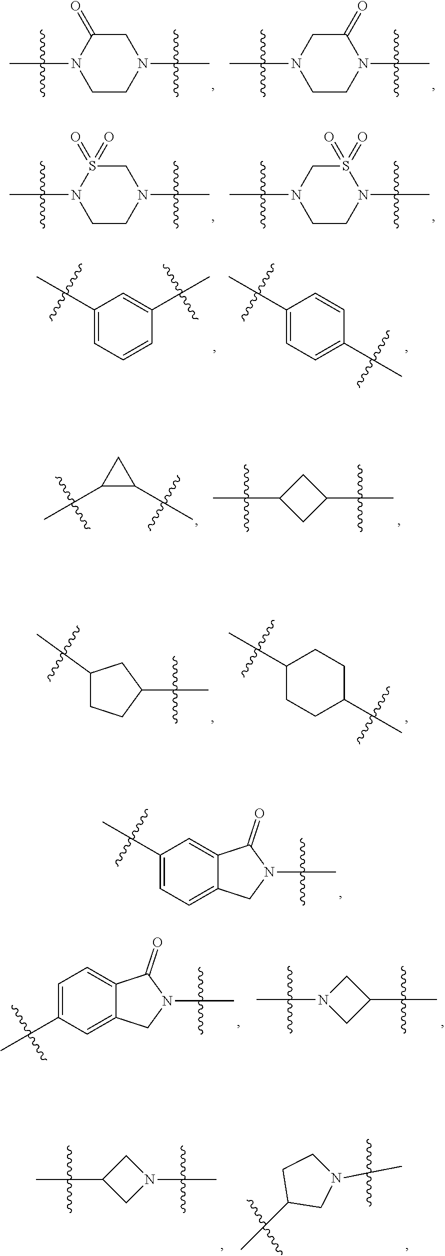 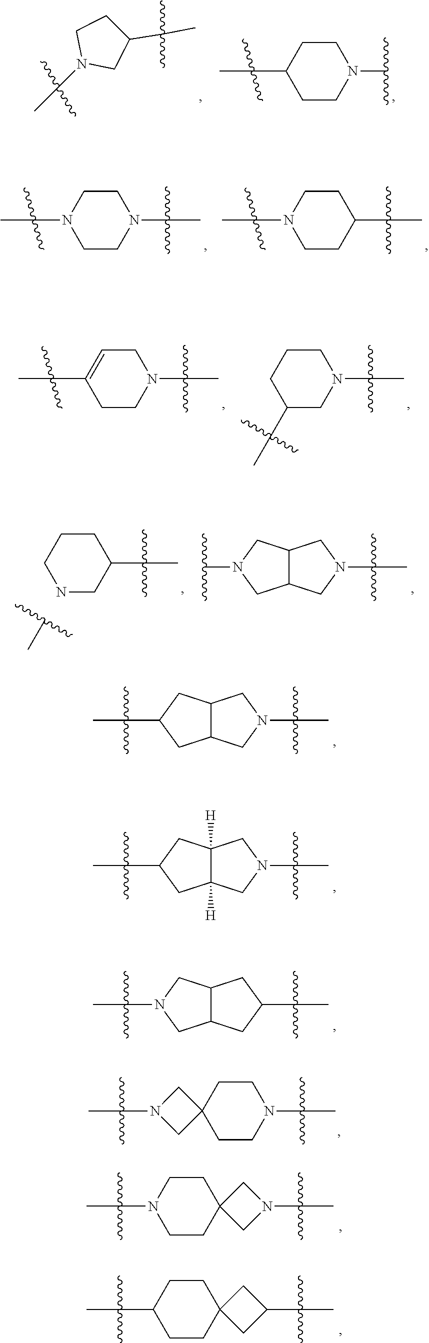 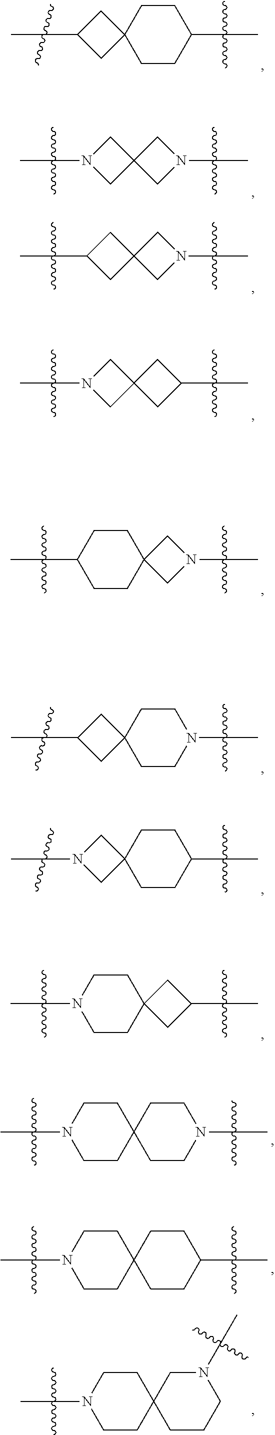 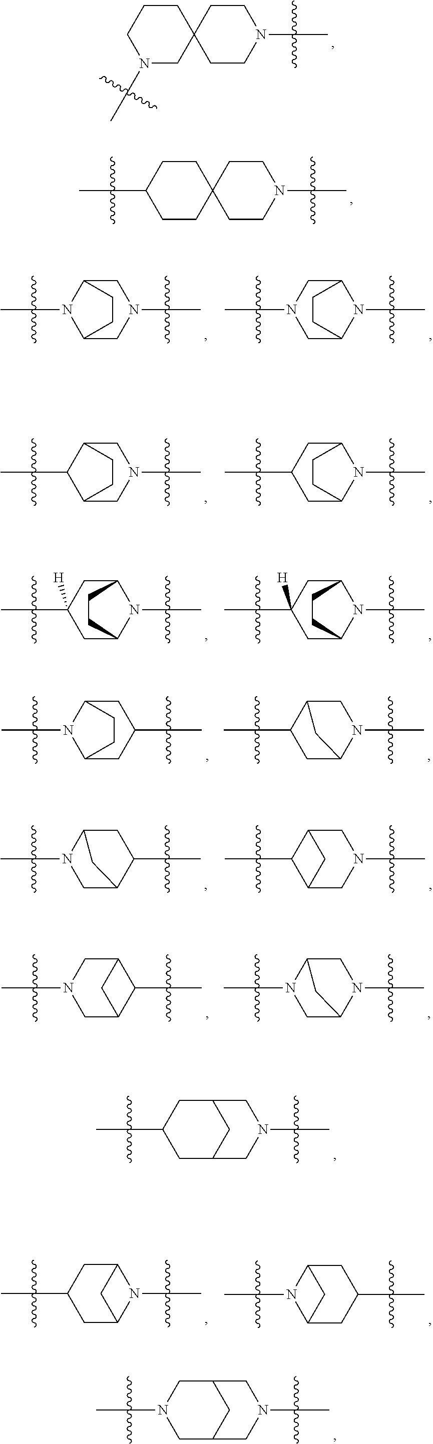 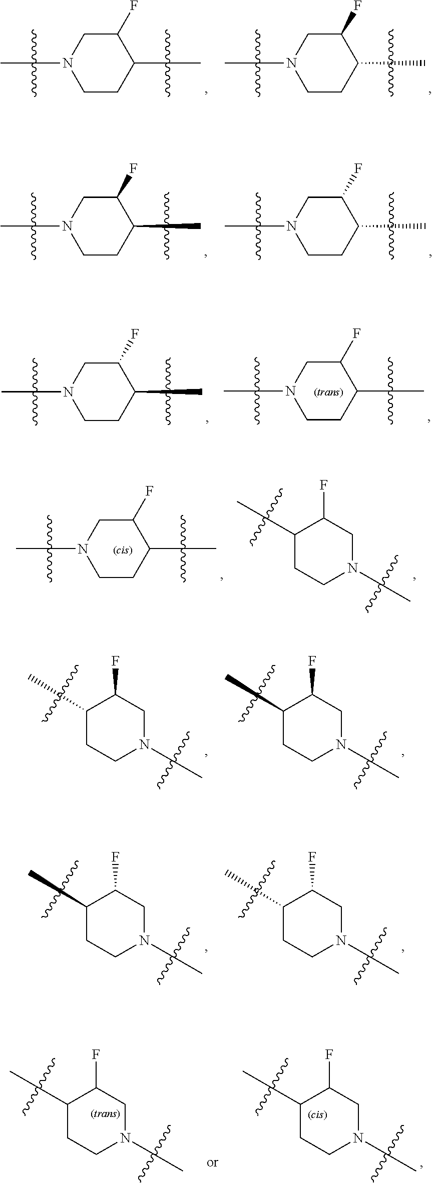
which, when substituted, is optionally further substituted with 0 to 4 substituents selected from H, F, CF₃, methyl, ═O, hydroxymethyl, COOH, CN or NH₂;
B is selected from one of the structural fragments shown in Table B-a;
K is selected from one of the structural fragments shown in Table K-a.

6. The compound or the stereoisomer, deuterated compound, solvate, prodrug, metabolite, pharmaceutically acceptable salt, or co-crystal thereof according to claim 5, wherein,
L is selected from a bond, -Ak1-, -Ak1-Ak2-, -Ak1-Ak2-Ak3-, -Ak1-Ak2-Ak3-Ak4-, -Ak1-Ak2-Ak3-Ak4-Ak5-, -Ak1-Ak2-Ak3-Ak4-Ak5-Ak6-, -Cy1-, -Cy1-Ak1-, -Cy1-Ak1-Ak2-, -Cy1-Ak1-Ak2-Ak3-, -Cy1-Ak1-Ak2-Ak3-Ak4-, -Cy1-Cy2-, -Cy1-Ak1-Cy2-, -Cy1-Cy2-Ak2-, -Cy1-Ak1-Cy2-Ak2-, -Cy1-Ak1-Cy2-Ak2-Ak3-, -Cy1-Ak1-Cy2-Ak2-Ak3-Ak4-, -Cy1-Cy2-Ak2-Ak3-, -Cy1-Cy2-Ak2-Ak3-Ak4-, -Cy1-Ak1-Ak2-Cy3-, -Cy1-Ak1-Ak2-Cy3-Ak3-, -Cy1-Cy2-Cy3-, -Cy1-Ak1-Cy2-Cy3-, -Cy1-Cy2-Ak2-Cy3-, -Cy1-Cy2-Cy3-Ak3-, -Cy1-Ak1-Cy2-Cy3-Ak3-, -Cy1-Cy2-Ak2-Cy3-Ak3-, -Cy1-Ak1-Cy2-Ak2-Cy3-, -Cy1-Ak1-Cy2-Ak2-Cy3-Ak3-, -Cy1-Cy2-Cy3-Ak3-Ak4-, -Cy1-Cy2-Cy3-Ak3-Cy4-, -Cy1-Cy2-Cy3-Cy4-, -Cy1-Ak1-Cy2-Cy3-Cy4-, -Cy1-Cy2-Ak2-Cy3-Cy4-, -Cy1-Cy2-Cy3-Ak3-Cy4-, -Cy1-Cy2-Cy3-Cy4-Ak4-, -Cy1-Ak1-Cy2-Ak2-Cy3-Ak3-Cy4-, -Cy1-Ak1-Cy2-Ak2-Cy3-Cy4-, -Ak1-Cy2-, -Ak1-Cy2-Cy3-, -Ak1-Ak2-Cy3-, -Ak1-Ak2-Cy3-Cy4-, -Ak1-Cy2-Ak2-Cy3-, -Ak1-Cy2-Cy3-Ak3-Cy4-, -Ak1-Cy2-Cy3-Cy4-Ak4-Cy5-, -Ak1-Cy2-Ak2-, -Cy1-Cy2-Cy3-Ak3-Ak4-Ak5-, -Cy1-Cy2-Ak2-Cy3-Ak3-Ak4-Ak5-, -Cy1-Ak1-Cy2-Ak2-Ak3-Ak4-Ak5-, -Cy1-Cy2-Cy3-Cy4-Ak4-Ak5-, -Cy1-Ak1-Ak2-Ak3-Ak4-Ak5-, -Ak1-Cy2-Ak2-Ak3-Ak4-Ak5-, -Ak1-Cy2-Ak2-Ak3-Ak4-, -Ak1-Cy2-Ak2-Ak3-, -Ak1-Ak2-Ak3-Ak4-Ak5-, -Ak1-Ak2-Ak3-Ak4-Ak5-Ak6-, or -Ak1-Ak2-Ak3-Ak4-Ak5-Ak6-Ak7-.

7. The compound or the stereoisomer, deuterated compound, solvate, prodrug, metabolite, pharmaceutically acceptable salt, or co-crystal thereof according to claim 1, wherein,
L is selected from a bond or a group shown in Table B-1, wherein the left side of the group is linked to B.

8. The compound or the stereoisomer, deuterated compound, solvate, prodrug, metabolite, pharmaceutically acceptable salt, or co-crystal thereof according to claim 7, wherein,
K is selected from one of the following structural fragments:

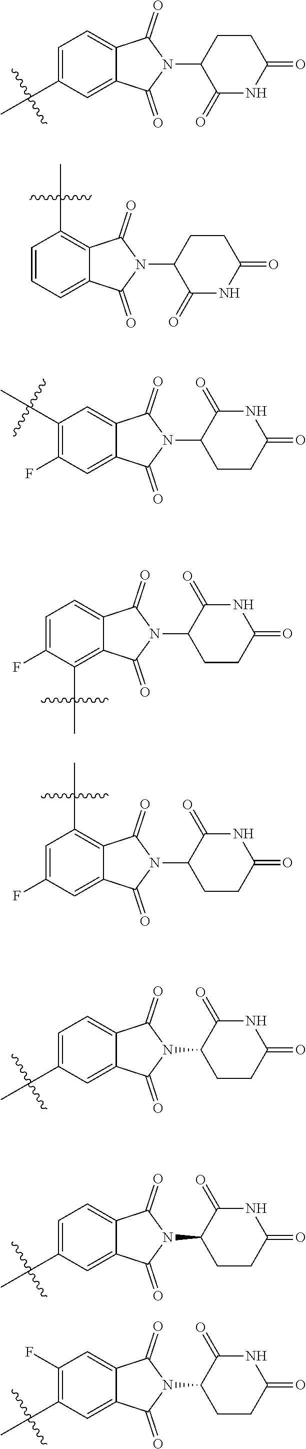

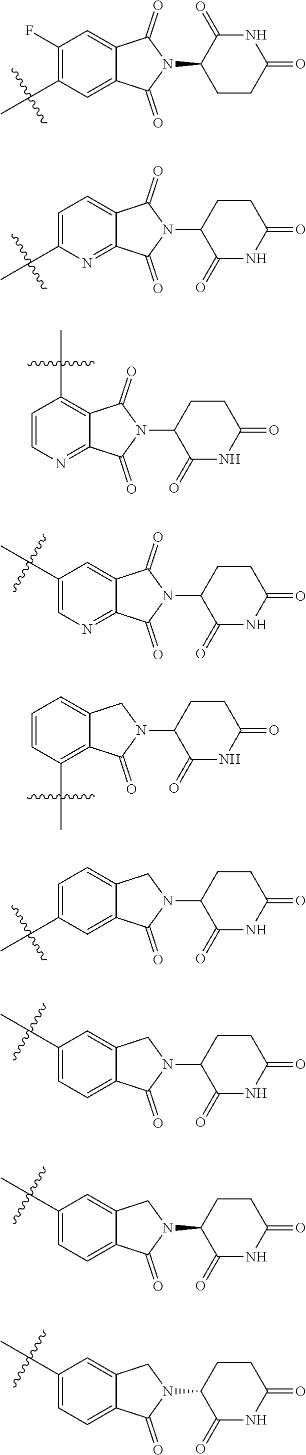

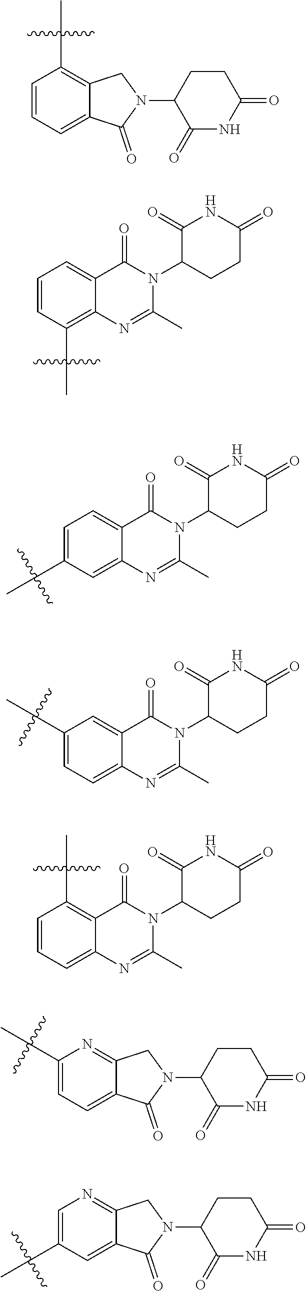

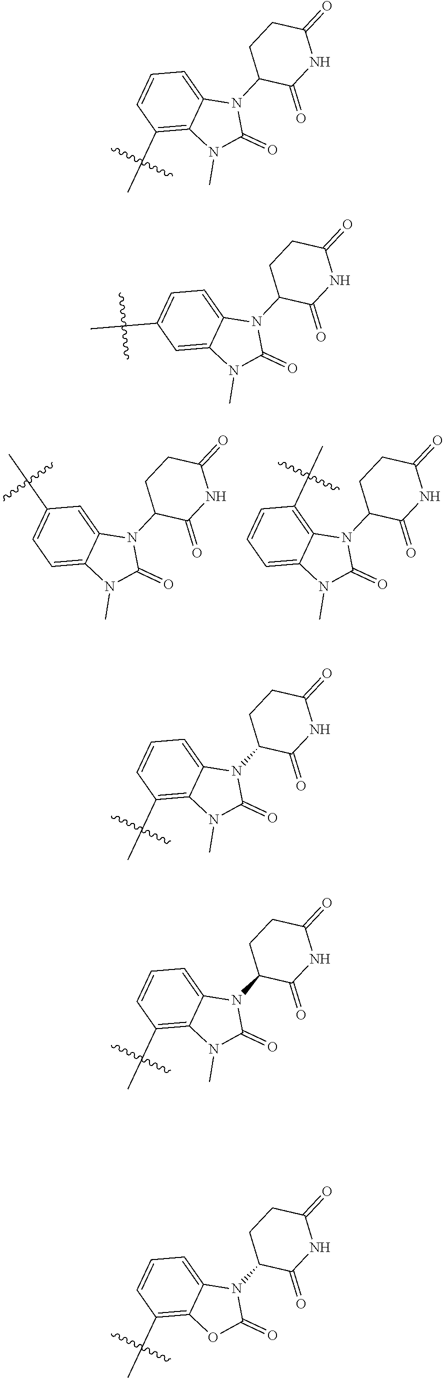

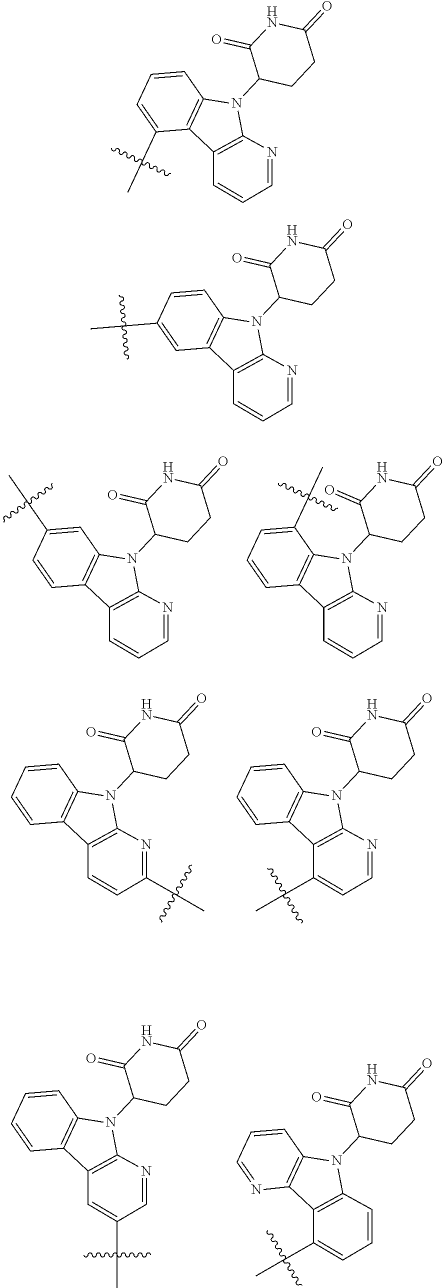

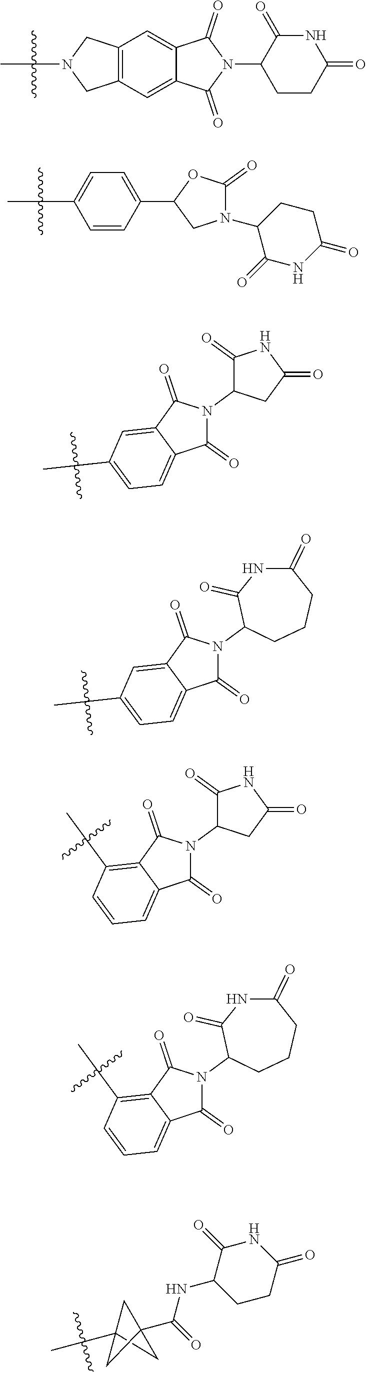

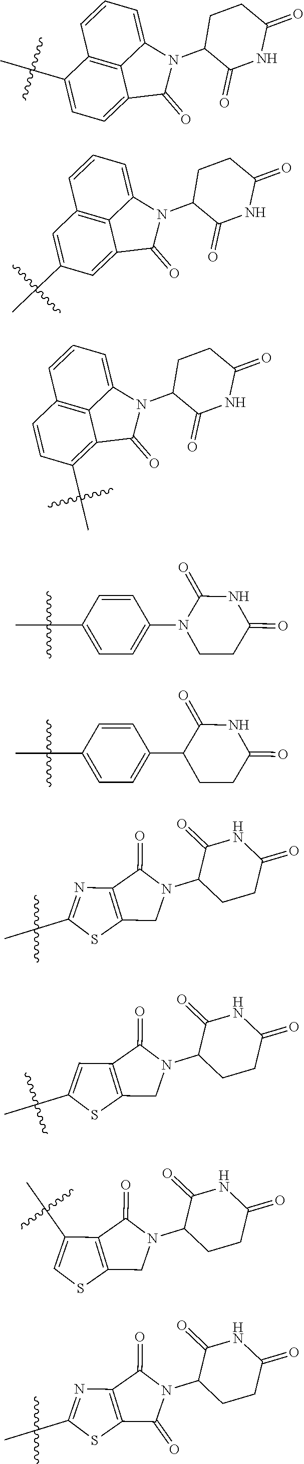

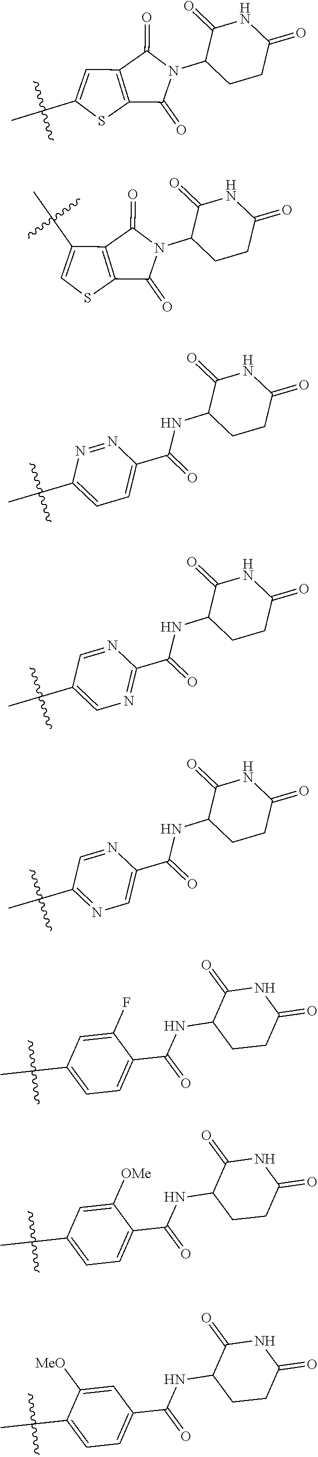

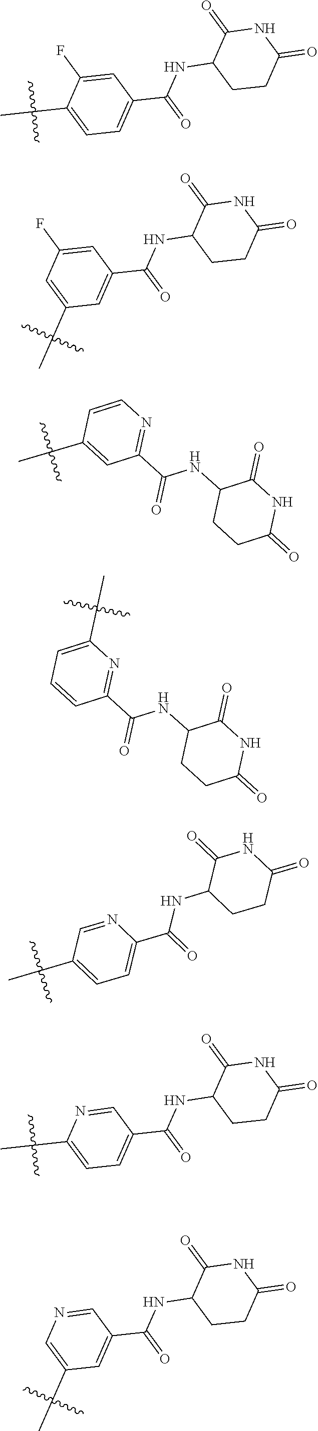

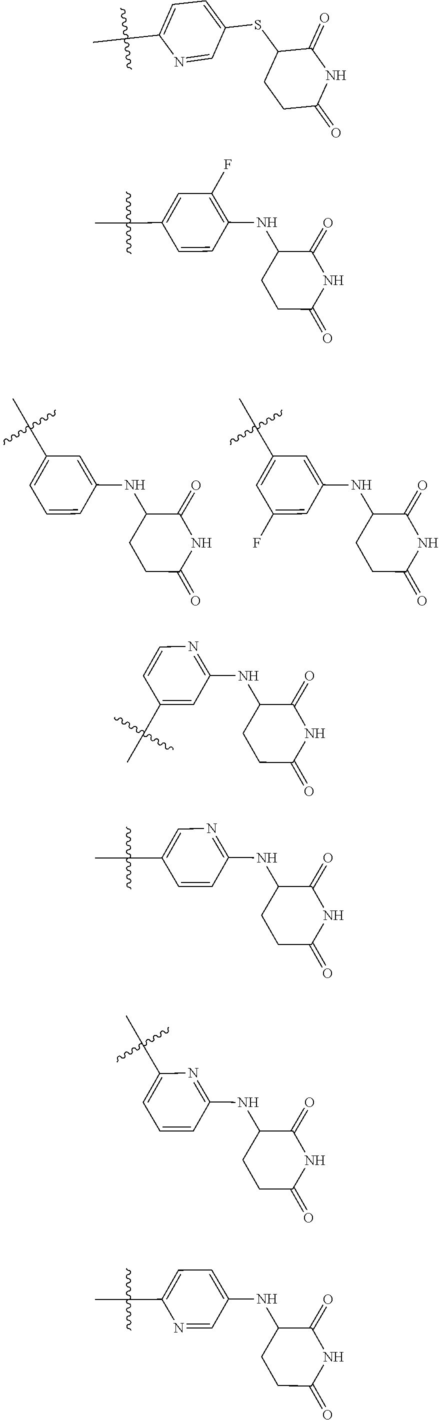

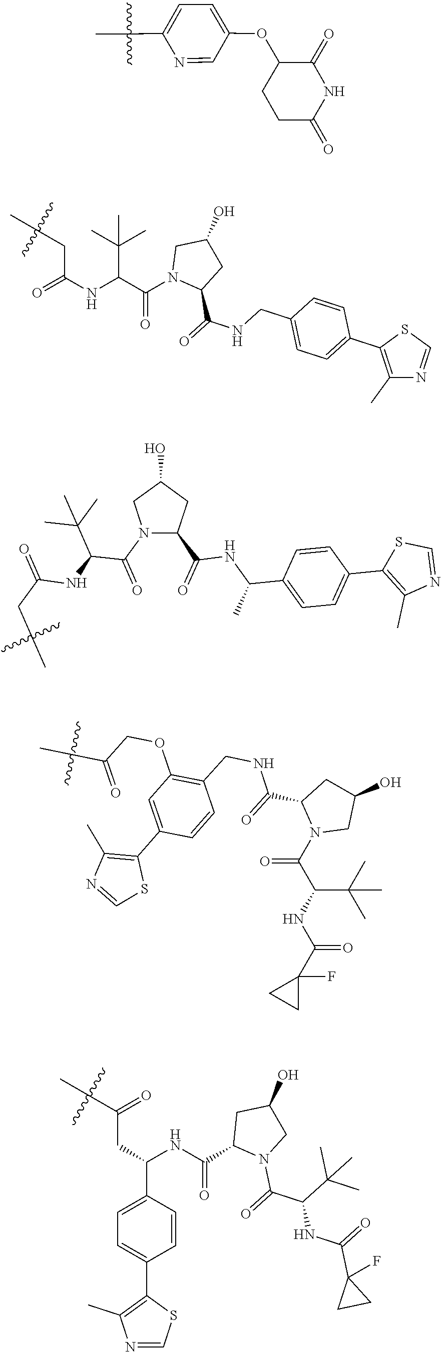 [SMILES: *c1ccc(OC2CCC(=O)NC2=O)cn1 ; *CC(=O)NC(C(=O)N1C[C@H](O)C[C@H]1C(=O)NCc1ccc(-c2scnc2C)cc1)C(C)(C)C ; *CC(=O)N[C@H](C(=O)N1C[C@H](O)C[C@H]1C(=O)N[C@@H](C)c1ccc(-c2scnc2C)cc1)C(C)(C)C ; *C(=O)COc1cc(-c2scnc2C)ccc1CNC(=O)[C@@H]1C[C@@H](O)CN1C(=O)[C@@H](NC(=O)C1(F)CC1)C(C)(C)C ; *C(=O)C[C@H](NC(=O)[C@@H]1C[C@@H](O)CN1C(=O)[C@@H](NC(=O)C1(F)CC1)C(C)(C)C)c1ccc(-c2scnc2C)cc1]

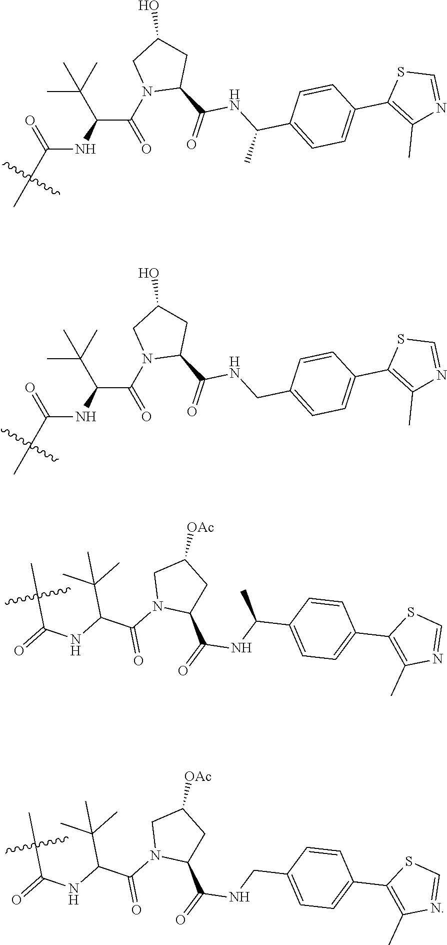 [SMILES: *C(=O)N[C@H](C(=O)N1C[C@H](O)C[C@H]1C(=O)N[C@@H](C)c1ccc(-c2scnc2C)cc1)C(C)(C)C ; *C(=O)N[C@H](C(=O)N1C[C@H](O)C[C@H]1C(=O)NCc1ccc(-c2scnc2C)cc1)C(C)(C)C ; *C(=O)NC(C(=O)N1C[C@H](OC(C)=O)C[C@H]1C(=O)N[C@@H](C)c1ccc(-c2scnc2C)cc1)C(C)(C)C ; *C(=O)NC(C(=O)N1C[C@H](OC(C)=O)C[C@H]1C(=O)NCc1ccc(-c2scnc2C)cc1)C(C)(C)C]

9. The compound or the stereoisomer, deuterated compound, solvate, prodrug, metabolite, pharmaceutically acceptable salt, or co-crystal thereof according to claim 1, wherein the compound is selected from a compound of general formula (Ia) 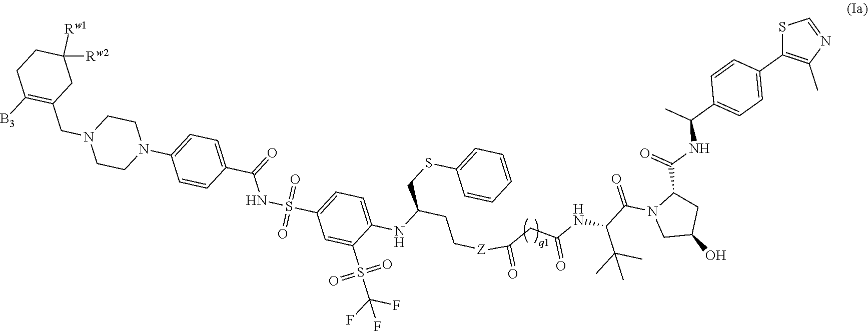
q1 is selected from 1, 2, 3, 4, 5, 6, 7 or 8;
B₃ is selected from 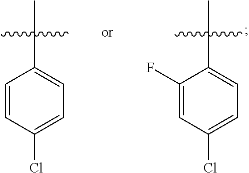 [SMILES: *c1ccc(Cl)cc1 ; *c1ccc(Cl)cc1F]
R^{w1} and R^{w2} are selected from methyl;
or R^{w1} and R^{w2} are directly connected to form, together with the carbon atom to which they are attached, cyclopropyl;
Z is selected from 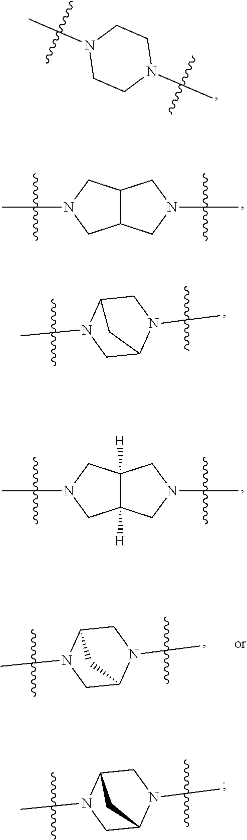 [SMILES: *N1CCN(*)CC1 ; *N1CC2CN(*)CC2C1 ; *N1CC2CC1CN2* ; *N1C[C@H]2CN(*)C[C@H]2C1 ; *N1C[C@H]2C[C@@H]1CN2* ; *N1C[C@@H]2C[C@H]1CN2*]
when Z is selected from 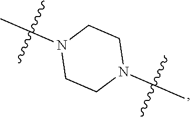 [SMILES: *N1CCN(*)CC1]
and R^{w1} and R^{w2} are selected from methyl, B₃ is selected from

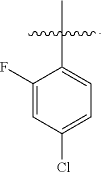 [SMILES: *c1ccc(Cl)cc1F]

10. The compound or the stereoisomer, deuterated compound, solvate, prodrug, metabolite, pharmaceutically acceptable salt, or co-crystal thereof according to claim 1, wherein the compound is selected from a compound of general formula (Ib) 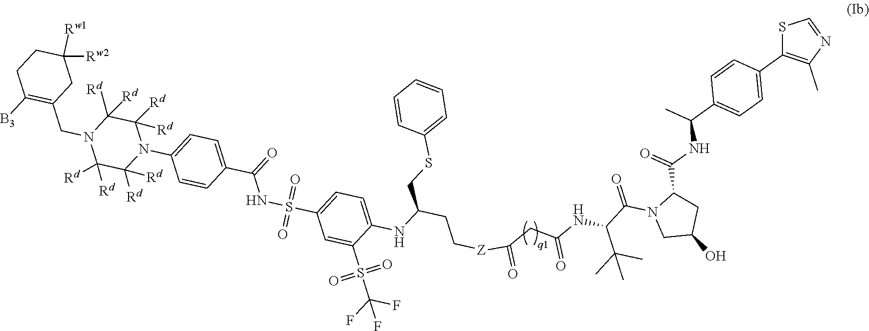
q1 is selected from 1, 2, 3, 4, 5, 6, 7 or 8;
B₃ is selected from 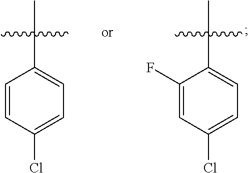 [SMILES: *c1ccc(Cl)cc1 ; *c1ccc(Cl)cc1F]
R^{w1} and R^{w2} are selected from methyl;
or R^{w1} and R^{w2} are directly connected to form, together with the carbon atom to which they are attached, cyclopropyl;
Z is selected from 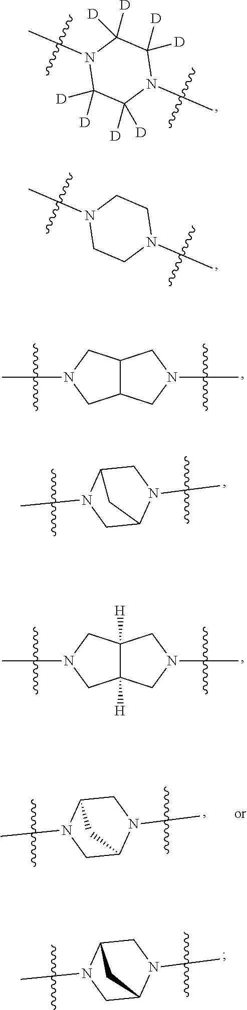
each R^{d} is independently selected from H or deuterium;
the compound of general formula (Ib) has at least one deuterium.

11. The compound or the stereoisomer, deuterated compound, solvate, prodrug, metabolite, pharmaceutically acceptable salt, or co-crystal thereof according to claim 1, wherein the compound is selected from one of the structures shown in Table P-1.

12. A pharmaceutical composition, comprising the compound or the stereoisomer, deuterated compound, solvate, prodrug, metabolite, pharmaceutically acceptable salt, or co-crystal thereof according to claim 1, and a pharmaceutically acceptable carrier, wherein preferably, the pharmaceutical composition comprises 1-1500 mg of the compound or the stereoisomer, deuterated compound, solvate, prodrug, metabolite, pharmaceutically acceptable salt, or co-crystal thereof.

13. (canceled)

14. (canceled)

15. (canceled)

16. A method for treating a disease in a mammal, the method comprising administering to a subject a therapeutically effective amount of the compound or the stereoisomer, deuterated compound, solvate, prodrug, metabolite, pharmaceutically acceptable salt, or co-crystal thereof according to claim 1.

17. The pharmaceutical composition according to claim 12, wherein, the pharmaceutical composition comprises 1-1500 mg of the compound or the stereoisomer, deuterated compound, solvate, prodrug, metabolite, pharmaceutically acceptable salt, or co-crystal thereof.

18. The method according to claim 16, wherein, the therapeutically effective amount is 1-1500 mg.

19. The method according to claim 16, wherein, the disease is cancer.
